(19) United States
(12) Patent Application Publication
(10) Pub. No.: US 2026/0131009 A1
(43) Pub. Date: May 14, 2026

(54) BIFUNCTIONAL PI3K-ALPHA INHIBITORS AND USES THEREOF

(71) Applicant: Relay Therapeutics, Inc., Cambridge, MA (US)

(72) Inventors: Alessandro BOEZIO, Cambridge, MA (US); Brandi M. HUDSON, Cambridge, MA (US); Thomas H. MCLEAN, Cambridge, MA (US); Demetri T. MOUSTAKAS, Cambridges, MA (US)

(21) Appl. No.: 18/706,471

(22) PCT Filed: Nov. 3, 2022
(86) PCT No.: PCT/US2022/079223

§ 371 Date: May 1, 2024

Related U.S. Application Data
(60) Provisional application No. 63/263,498, filed on Nov. 3, 2021.

(51) Int. Cl. A61K 47/55; A61K 47/54; C07D 405/14; C07D 409/14; C07D 417/14; C07D 487/04

(52) U.S. Cl. CPC A61K 47/55; A61K 47/545; C07D 405/14; C07D 409/14; C07D 417/14; C07D 487/04

(57) ABSTRACT

The present disclosure relates to novel bifunctional compounds and pharmaceutical compositions thereof, and methods for degrading PI3K-alpha and/or inhibiting the activity of PI3K-alpha enzymes with the compounds and compositions of the disclosure. The present disclosure further relates to, but is not limited to, methods for treating disorders associated with PI3K-alpha signaling with the compounds and compositions of the disclosure.

### CROSS-REFERENCE TO RELATED APPLICATIONS

This application claims the benefit of U.S. Provisional Application No. 63/263,498 filed on Nov. 3, 2021, the entirety of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

The UPP is used to induce selective protein degradation, including use of fusion proteins to artificially ubiquitinate target proteins and synthetic small-molecule probes to induce proteasome-dependent degradation. Bifunctional compounds, composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1): 40-46).

In view of the above, bifunctional inhibitors and/or degraders of PI3Kα would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY

In some embodiments, the present disclosure provides a compound of formula I:

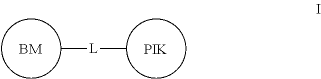

or a pharmaceutically acceptable salt thereof, wherein each of BM, L, and PIK is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent.

In some embodiments, the present disclosure provides a method of treating a PI3Kα-mediated disorder comprising administering to a patient in need thereof a compound of formula I, or composition comprising said compound.

In some embodiments, the present disclosure provides a process for providing a compound of formula I, or synthetic intermediates thereof.

In some embodiments, the present disclosure provides a process for providing pharmaceutical compositions comprising compounds of formula I.

### DETAILED DESCRIPTION

### 1. General Description of Certain Embodiments of the Disclosure

Compounds of the present disclosure, and pharmaceutical compositions thereof, are useful as inhibitors and/or degraders of PI3Kα. In some embodiments, the present disclosure provides a compound of formula I:

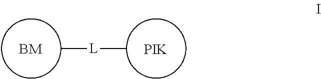

or a pharmaceutically acceptable salt thereof, wherein:
PIK is a first PI3K binding moiety capable of binding to PI3Kα;
L is a bivalent moiety that connects PIK to BM; and
BM is a binding motif LBM, PIK2, or T, wherein:
   LBM is an E3 ubiquitin ligase binding moiety;
   PIK2 is a second PI3K binding moiety capable of binding to PI3Kα;
   T is R^{A*} or R^{B*} substituted by t instances of R^{TC};
R^{A*} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
R^{B*} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{TC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
   t is 0, 1, 2, 3, 4, or 5.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, allenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ allyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent allyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent allenyl group. A substituted allenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

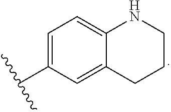 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

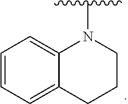 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

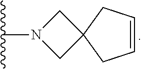 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent allyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

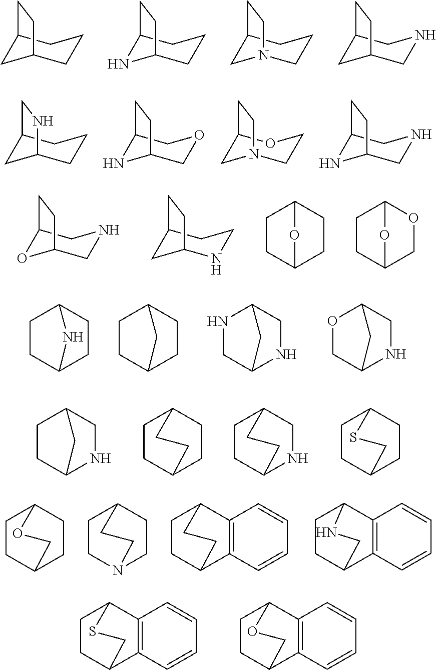

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁-Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include allali metal, allaline earth metal, ammonium and N⁺(C₁₋₄alkyl)₄ salts. Representative allali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to PI3Kα signaling, or one or more symptoms thereof, prevent the advancement of conditions or symptoms related to afflictions related to PI3Kα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to PI3Kα signaling activity or that may otherwise be relieved by the compounds and/or compositions of the disclosure.

As used herein, the term “degrader” is defined as a heterobifunctional or monovalent compound that binds to and/or inhibits both an PI3Kα and an E3 ligase with measurable affinity resulting in the ubiqitination and subsequent degradation of the PI3Kα. In certain embodiments, a degrader has an DC50 of less than about 50 pM, less than about 1 pM, less than about 500 nM, less than about 100 nM, less than about 10 nM, or less than about 1 nM. As used herein, the term “monovalent” refers to a degrader compound without an appended E3 ligase binding moiety.

A compound of the present disclosure may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al, Angew. Chem. Int. Ed. 2002, 41, 2596-99 and Sun et al, Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FF, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Fissamine rhodamine B, Marina Blue, Methoxy coumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750; 4,709,016; 5,360,819; 5,516,931; 5,602,273; 5,603,104; 5,610,020; and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

### 3. Description of Exemplary Embodiments

As described above, in some embodiments, the present disclosure provides a compound of formula I:

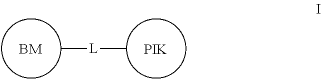

or a pharmaceutically acceptable salt thereof, wherein:
PIK is a first PI3K binding moiety capable of binding to PI3Kα;
L is a bivalent moiety that connects PIK to BM; and
BM is a binding motif LBM, PIK2, or T, wherein:
   LBM is an E3 ubiquitin ligase binding moiety;
   PIK2 is a second PI3K binding moiety capable of binding to PI3Kα;
   T is R^{A*} or R^{B*} substituted by t instances of R^{TC};
R^{A*} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
R^{B*} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{TC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
   t is 0, 1, 2, 3, 4, or 5.

In some embodiments, the present disclosure provides a compound of Formula I, wherein each of PIK, BM, LBM, PIK2, T, R^{A*}, R^{B*}, R^{TC}, R, and t is as defined below, and described in embodiments herein, both singly and in combination.

### PI3K Binding Moiety (PIK)

As defined generally above, PIK is a first PI3K binding moiety capable of binding to PI3Kα.

In certain embodiments, PIK is a PI3K binding moiety of formula I-a0:

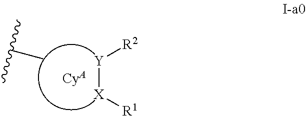

or a pharmaceutically acceptable salt thereof, wherein each of X, Y, Cy^{A}, R¹, and R² is as defined in embodiments and classes and subclasses herein.

In certain embodiments, PIK is a PI3K binding moiety of formula I-b0:

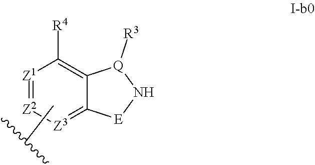

or a pharmaceutically acceptable salt thereof, wherein each of E, Q, R³, R⁴, Z¹, Z², and Z³ is as defined in embodiments and classes and subclasses herein.

In certain embodiments, PIK is a PI3K binding moiety of formula I-c0:

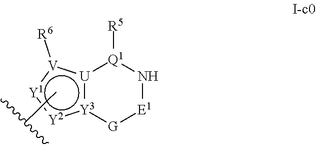

or a pharmaceutically acceptable salt thereof, wherein each of E¹, G, Q¹, R⁵, R⁶, U, V, Y¹, Y², and Y³ is as defined in embodiments and classes and subclasses herein.

In certain embodiments, PIK is a PI3K binding moiety of formula I-d0 or I-d00:

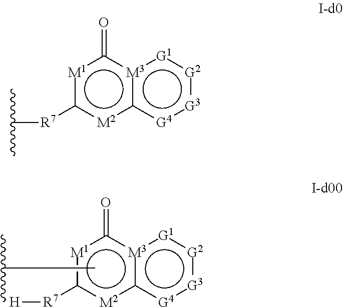

or a pharmaceutically acceptable salt thereof, wherein each of G¹, G², G³, G⁴, M¹, M², M³, and R⁷ is as defined in embodiments and classes and subclasses herein.

In certain embodiments, PIK is an PI3K binding moiety of formula I-a0, 1-b0, I-c0, I-d0, or I-d00:

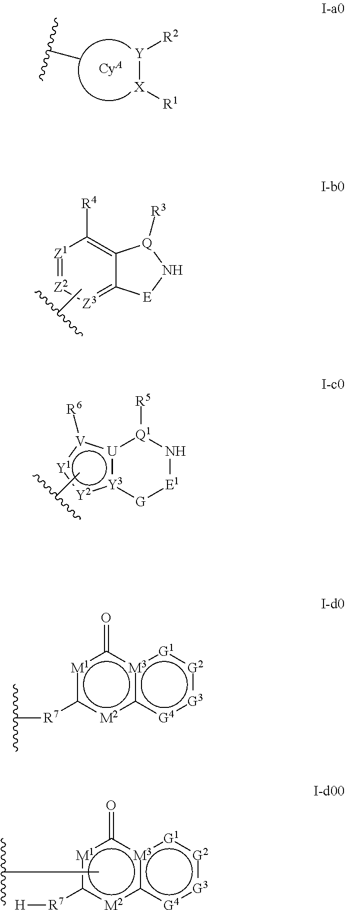

or a pharmaceutically acceptable salt thereof, wherein each of X, Y, Cy^{A}, R¹, R², E, Q, R³, R⁴, Z¹, Z², Z³, E¹, G, Q¹, R⁵, R⁶, U, V, Y¹, Y², Y³, G¹, G², G³, G⁴, M¹, M², M³, and R⁷ is as defined in embodiments and classes and subclasses herein.

In certain embodiments, the present disclosure provides a compound of formula I, in which PIK is a PI3K binding moiety of formula I-a0, thereby forming a compound of formula I-a:

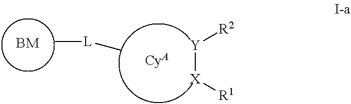

or a pharmaceutically acceptable salt thereof, wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA}; or
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloallylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, r¹, r², r³, r⁴, r⁵, and r⁶ is independently 0, 1, 2, 3, 4, or 5.

As defined generally above, X is C, CH, C(R^{X}), or N. In some embodiments, X is C.

In some embodiments, X is CH. In some embodiments, X is C(R^{X}). In some embodiments, X is N. In some embodiments, X is CH or C(R^{X}). In some embodiments, X is CH or N. In some embodiments, X is C(R^{X}) or N. In some embodiments, X is selected from the groups depicted in the compounds in Table 1.

As defined generally above, Y is C, CH, C(R^{Y}), or N. In some embodiments, Y is C.

In some embodiments, Y is CH. In some embodiments, Y is C(R^{Y}). In some embodiments, Y is N. In some embodiments, Y is CH or C(R^{Y}). In some embodiments, Y is CH or N. In some embodiments, Y is C(R^{Y}) or N. In some embodiments, Y is selected from the groups depicted in the compounds in Table 1.

As defined generally above, R¹ is -L¹-R^{1A}. In some embodiments, R¹ is -L¹-R^{1A}. In some embodiments, R¹ is —R^{1A}.

In some embodiments, R¹ (i.e. -L¹-R^{1A} taken together) is

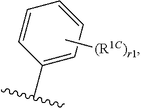

wherein R^{1C} and r¹ are as defined in the embodiments and classes and subclasses herein. In some embodiments, R¹ (i.e. -L¹-R^{1A} taken together) is

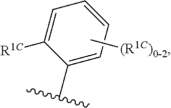

wherein R^{1C} is as defined in the embodiments and classes and subclasses herein. In some embodiments, R¹ (i.e. -L¹-R^{1A} taken together) is

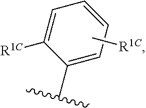

wherein R^{1C} is as defined in the embodiments and classes and subclasses herein. In some embodiments, R¹ (i.e. -L¹-R^{1A} taken together) is

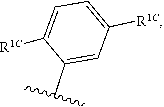

wherein R^{1C} is as defined in the embodiments and classes and subclasses herein. In some embodiments, R¹ (i.e. -L¹-R^{1A} taken together) is

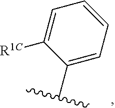

wherein R^{1C} is as defined in the embodiments and classes and subclasses herein.

In some embodiments, R¹ (i.e. -L¹-R^{1A} taken together) is

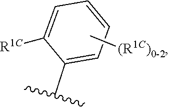

wherein each instance of R^{1C} is independently halogen, —CN, —O-(optionally substituted C₁₋₆ aliphatic), or an optionally substituted C₁₋₆ aliphatic. In some embodiments, R¹ (i.e. -L¹-R^{1A} taken together) is

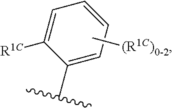

wherein each instance of R^{1C} is independently halogen or C₁₋₃ aliphatic optionally substituted with 1-3 halogen. In some embodiments, R¹ (i.e. -L¹-R^{1A} taken together) is

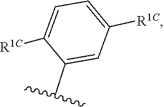

wherein each instance of R^{1C} is independently halogen or C₁₋₃ aliphatic optionally substituted with 1-3 halogen. In some embodiments, R¹ (i.e. -L¹-R^{1A} taken together) is

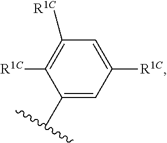

wherein each instance of R^{1C} is independently halogen or C₁₋₃ aliphatic optionally substituted with 1-3 halogen. In some embodiments, R¹ (i.e. -L¹-R^{1A} taken together) is

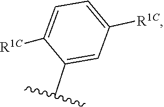

wherein each instance of R^{1C} is independently fluorine, chlorine, —CH₃, —CHF₂, or —CF₃. In some embodiments, R¹ (i.e. -L¹-R^{1A} taken together) is

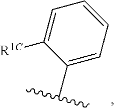

wherein R^{1C} is halogen or C₁₋₃ aliphatic optionally substituted with 1-3 halogen.

In some embodiments, R¹ (i.e. -L¹-R^{1A} taken together) is

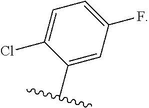 [SMILES: *c1cc(F)ccc1Cl]

In some embodiments, R¹ (i.e. -L¹-R^{1A} taken together)

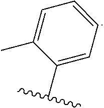 [SMILES: *c1ccccc1C]

In some embodiments, R¹ (i.e. -L¹-R^{1A} taken together) is

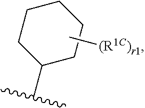

wherein R^{1C} and r¹ are as defined in the embodiments and classes and subclasses herein. In some embodiments, R¹ (i.e. -L¹-R^{1A} taken together) is

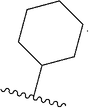 [SMILES: *C1CCCCC1]

In some embodiments, R¹ (i.e. -L¹-R^{1A} taken together) is

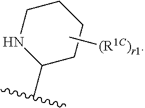

In some embodiments, R¹ (i.e. -L¹-R^{1A} taken together) is

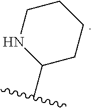 [SMILES: *C1CCCCN1]

In some embodiments, R¹ is selected from the groups depicted in the compounds in Table 1.

As defined generally above, R² is -L²-R^{2A}. In some embodiments, R² (i.e. -L²-R^{2A} taken together) is —N(R)C(O)—R^{2A}, —N(R)—R^{2A}, or —R^{2A}, wherein R and R^{2A} are as defined in the embodiments and classes and subclasses herein. In some embodiments, R² (i.e. -L²-R^{2A} taken together) is —N(R)C(O)—R^{2A} or —R^{2A}, wherein R and R^{2A} are as defined in the embodiments and classes and subclasses herein. In some embodiments, R² is —N(H)C(O)—R^{2A}, —N(H)—R^{2A}, or —R^{2A}.

In some embodiments, R² (i.e. -L²-R^{2A} taken together) is —N(R)C(O)—R^{2A}, wherein R and R^{2A} are as defined in the embodiments and classes and subclasses herein. In some embodiments, R² (i.e. -L²-R^{2A} taken together) is —N(H)C(O)—R^{2A}, wherein R^{2A} is as defined in the embodiments and classes and subclasses herein. In some embodiments, R² (i.e. -L²-R^{2A} taken together) is —N(H)C(O)—R^{2A}, wherein R^{2A} is R^{B} substituted by r² instances of R^{2C}. In some embodiments, R² (i.e. -L²-R^{2A} taken together) is —N(R)—R^{2A}, wherein R and R^{2A} are as defined in the embodiments and classes and subclasses herein. In some embodiments, R² is —R^{2A}.

In some embodiments, R² is —N(H)C(O)—R^{2A}, —N(H)C(O)N(H)—R^{2A}, —C(O)N(H)—R^{2A}, —N(H)—R^{2A}, —S(O)₂CH₂—R^{2A}, —CH₂S(O)₂—R^{2A}, or —C(H)(CH₃)OH. In some embodiments, R² is —N(H)C(O)—R^{2A}, —N(H)C(O)N(H)—R^{2A}, or —N(H)—R^{2A}. In some embodiments, R² is —C(O)N(H)—R^{2A}, —CH₂S(O)₂—R^{2A}, or —C(H)(CH₃)OH. In some embodiments, R² is —S(O)₂CH₂—R^{2A} or —CH₂S(O)₂—R^{2A}.

In some embodiments, R² is —N(H)C(O)N(H)—R^{2A}. In some embodiments, R² is —C(O)N(H)—R^{2A}. In some embodiments, R² is —N(H)—R^{2A}. In some embodiments, R² is —S(O)₂CH₂—R^{2A}. In some embodiments, R² is —CH₂S(O)₂—R^{2A}. In some embodiments, R² is —C(H)(CH₃)OH.

In some embodiments, R² (i.e. -L²-R^{2A} taken together) is

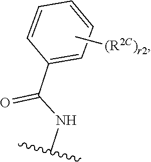

wherein R^{2C} and r² are as defined in the embodiments and classes and subclasses herein. In some embodiments, R² (i.e. -L²-R^{2A} taken together) is

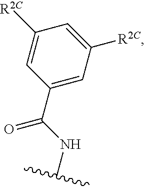

wherein R^{2C} is as defined in the embodiments and classes and subclasses herein.

In some embodiments, R² (i.e. -L²-R^{2A} taken together) is

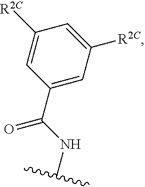

wherein each instance of R^{2C} is independently halogen, —CN, —O-(optionally substituted C₁₋₆ aliphatic), or an optionally substituted C₁₋₆ aliphatic. In some embodiments, R² (i.e. -L²-R^{2A} taken together) is

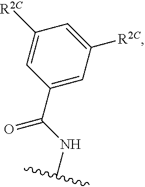

wherein each instance of R^{2C} is independently halogen or C₁₋₃ aliphatic optionally substituted with 1-3 halogen. In some embodiments, R² (i.e. -L²-R^{2A} taken together) is

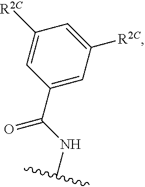

wherein each instance of R^{2C} is independently fluorine, chlorine, —CH₃, —CHF₂, or —CF₃. In some embodiments, R² (i.e. -L²-R^{2A} taken together) is

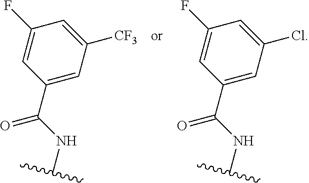 [SMILES: *NC(=O)c1cc(F)cc(C(F)(F)F)c1 ; *NC(=O)c1cc(F)cc(Cl)c1]

In some embodiments, R² (i.e. -L²-R^{2A} taken together) is

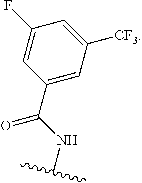 [SMILES: *NC(=O)c1cc(F)cc(C(F)(F)F)c1]

In some embodiments, R² (i.e. -L²-R^{2A} taken together) is

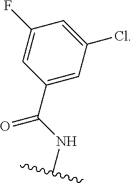 [SMILES: *NC(=O)c1cc(F)cc(Cl)c1]

In some embodiments, R² (i.e. -L²-R^{2A} taken together) is

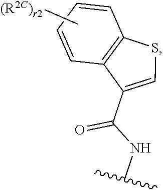

wherein R^{2C} and r² are as defined in the embodiments and classes and subclasses herein. In some embodiments, R² (i.e. -L²-R^{2A} taken together) is

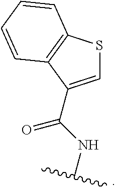 [SMILES: *NC(=O)c1csc2ccccc12]

In some embodiments, R² (i.e. -L²-R^{2A} taken together) is

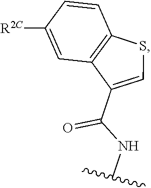

wherein R^{2C} is as defined in the embodiments and classes and subclasses herein.

In some embodiments, R² (i.e. -L²-R^{2A} taken together) is

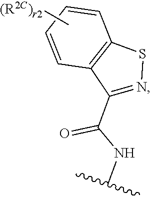

wherein R^{2C} and r² are as defined in the embodiments and classes and subclasses herein. In some embodiments, R² (i.e. -L²-R^{2A} taken together) is

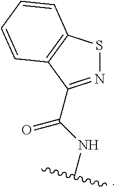 [SMILES: *NC(=O)c1nsc2ccccc12]

In some embodiments, R² (i.e. -L²-R^{2A} taken together) is

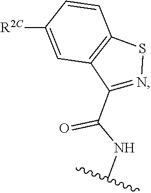

wherein R^{2C} is as defined in the embodiments and classes and subclasses herein.

In some embodiments, R² (i.e. -L²-R^{2A} taken together) is

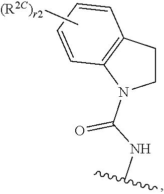

wherein R^{2C} and r² are as defined in the embodiments and classes and subclasses herein. In some embodiments, R² (i.e. -L²-R^{2A} taken together) is

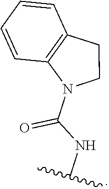 [SMILES: *NC(=O)N1CCc2ccccc21]

In some embodiments, R² (i.e. -L²-R^{2A} taken together) is

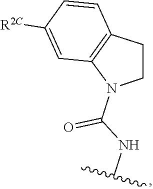

WHEREIN R^{2C} is as defined in the embodiments and classes and subclasses herein.

In some embodiments, R² (i.e. -L²-R^{2A} taken together) is

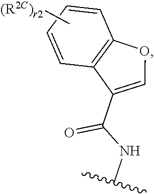

wherein R^{2C} and r² are as defined in the embodiments and classes and subclasses herein. In some embodiments, R² (i.e. -L²-R^{2A} taken together) is

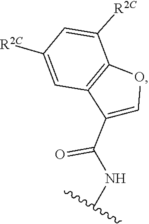

wherein R^{2C} is as defined in the embodiments and classes and subclasses herein.

In some embodiments, R² (i.e. -L²-R^{2A} taken together) is

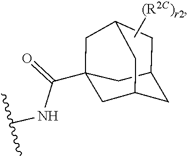

wherein R^{2C} and r² are as defined in the embodiments and classes and subclasses herein. In some embodiments, R² (i.e. -L²-R^{2A} taken together) is

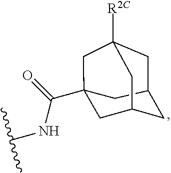

wherein R^{2C} is as defined in the embodiments and classes and subclasses herein.

In some embodiments, R² (i.e. -L²-R^{2A} taken together) is

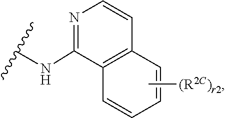

wherein R^{2C} and r² are as defined in the embodiments and classes and subclasses herein. In some embodiments, R² (i.e. -L²-R^{2A} taken together) is

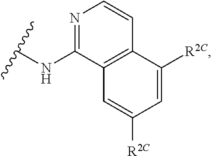

wherein R^{2C} is as defined in the embodiments and classes and subclasses herein.

In some embodiments, R² (i.e. -L²-R^{2A} taken together) is

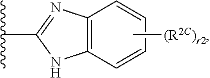

wherein R^{2C} and r² are as defined in the embodiments and classes and subclasses herein. In some embodiments, R² (i.e. -L²-R^{2A} taken together) is

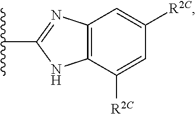

wherein R^{2C} is as defined in the embodiments and classes and subclasses herein.

In some embodiments, R² is

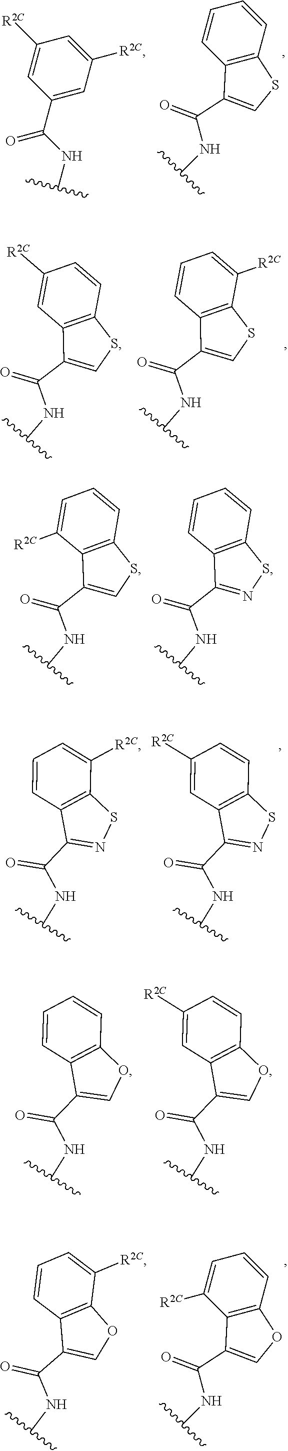

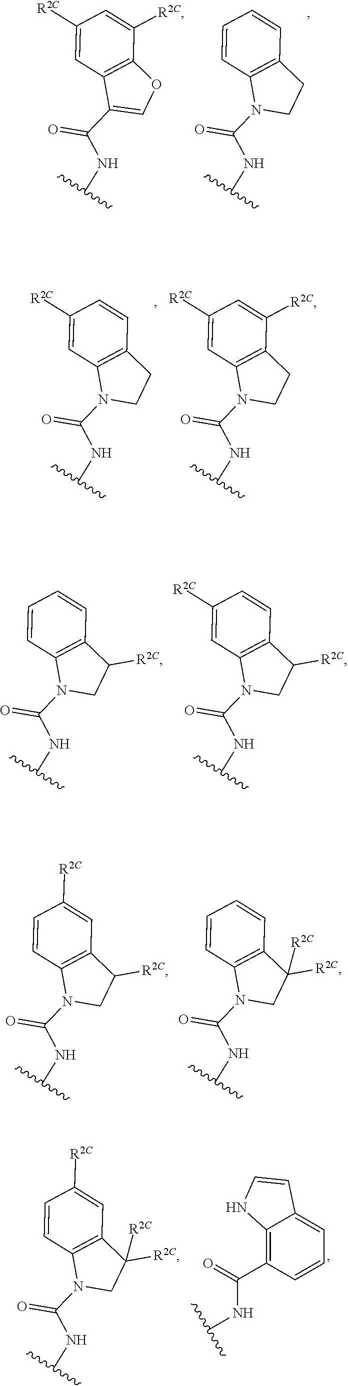

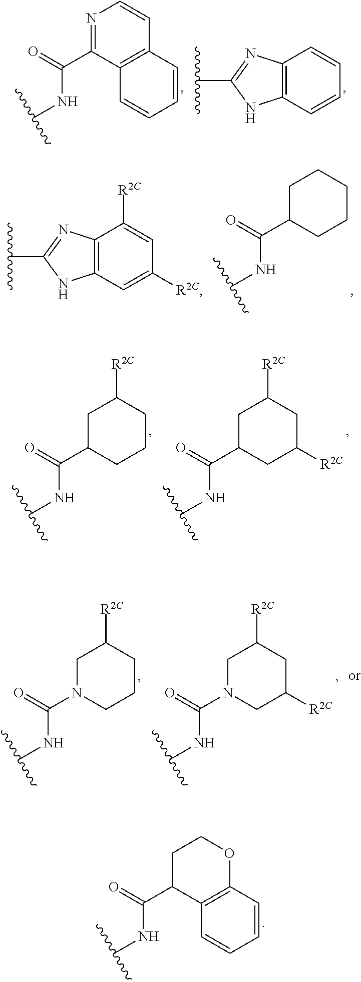

In some embodiments, R² is

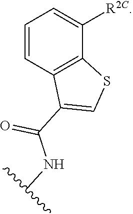

In some embodiments, R² is

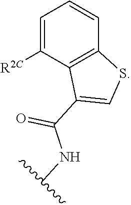

In some embodiments, R² is

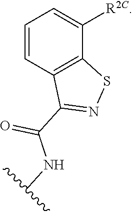

In some embodiments, R² is

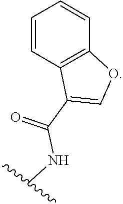 [SMILES: *NC(=O)c1coc2ccccc12]

In some embodiments, R² is

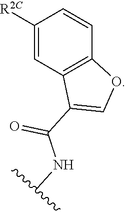

In some embodiments, R² is

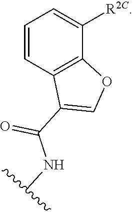

In some embodiments, R² is

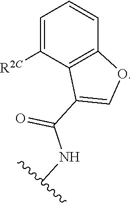

In some embodiments, R² is

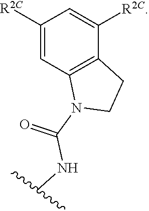

In some embodiments, R² is

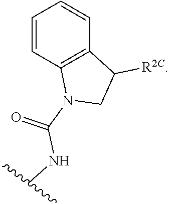

In some embodiments, R² is

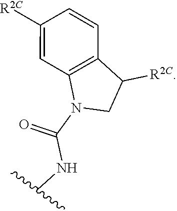

some embodiments, R² is

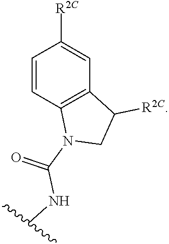

In some embodiments, R² is

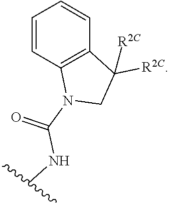

In some embodiments, R² is

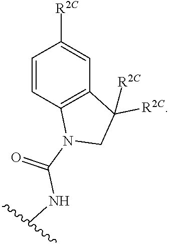

In some embodiments, R² is

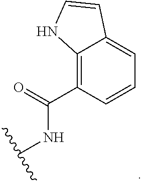 [SMILES: *NC(=O)c1cccc2cc[nH]c12]

In some embodiments, R² is

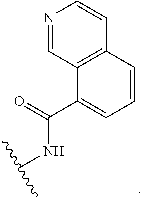 [SMILES: *NC(=O)c1cccc2ccncc12]

In some embodiments, R² is

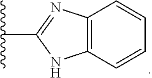 [SMILES: *c1nc2ccccc2[nH]1]

In some embodiments, R² is

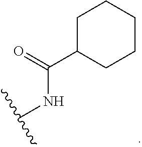 [SMILES: *NC(=O)C1CCCCC1]

In some embodiments, R² is

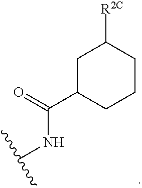

In some embodiments, R² is

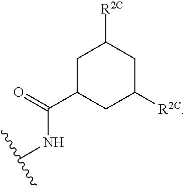

In some embodiments, R² is

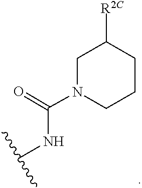

In some embodiments, R² is

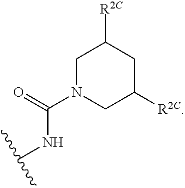

In some embodiments, R² is

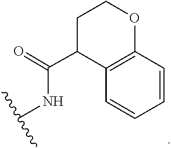 [SMILES: *NC(=O)C1CCOc2ccccc21]

In some embodiments, R² is selected from the groups depicted in the compounds in Table 1.

As defined generally above, R^{X} is -L^{X}-R^{XA}. In some embodiments, R^{X} is —R^{XA}.

In some embodiments, R^{X} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{X} is halogen, —CN, —OH, —O-(optionally substituted C₁₋₆ aliphatic), or an optionally substituted C₁₋₆ aliphatic. In some embodiments, R^{X} is halogen, —OH, or C₁₋₃ aliphatic optionally substituted with 1-3 halogen. In some embodiments, R^{X} is fluorine, chlorine, —OH, or —CH₃. In some embodiments, R^{X} is deuterium. In some embodiments, R^{X} is selected from the groups depicted in the compounds in Table 1.

As defined generally above, R^{Y} is -L^{Y}-R^{YA}. In some embodiments, R^{Y} is —R^{YA}.

In some embodiments, R^{Y} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Y} is halogen, —CN, —OH, —O-(optionally substituted C₁₋₆ aliphatic), or an optionally substituted C₁₋₆ aliphatic. In some embodiments, R^{Y} is halogen, —OH, or C₁₋₃ aliphatic optionally substituted with 1-3 halogen. In some embodiments, R^{Y} is fluorine, chlorine, —OH, or —CH₃. In some embodiments, R^{Y} is deuterium. In some embodiments, R^{Y} is selected from the groups depicted in the compounds in Table 1.

As defined generally above, each instance of R^{CyA} is independently -L^{CyA}-R^{CyAA}.

In some embodiments, each instance of R^{CyA} is independently —C(O)N(H)—R^{CyAA}, —C(O)N(H)CH₂—R^{CyAA}, or —R^{CyAA}. In some embodiments, each instance of R^{CyA} is independently —C(O)N(H)—R^{CyAA}. In some embodiments, each instance of R^{CyA} is independently —C(O)N(H)CH₂—R^{CyAA}. In some embodiments, each instance of R^{CyA} is independently —R^{CyAA}.

In some embodiments, each instance of R^{CyA} is independently

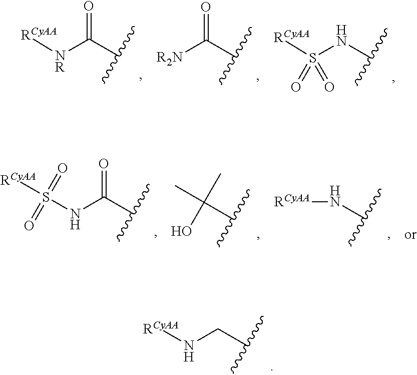

In some embodiments, each instance of R^{CyA} is independently

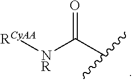

In some embodiments, each instance of R^{CyA} is independently

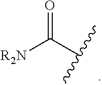

In some embodiments, each instance of R^{CyA} is independently

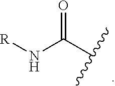

In some embodiments, each instance of R^{CyA} is independently

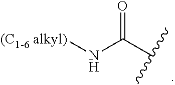

In some embodiments, each instance of R^{CyA} is

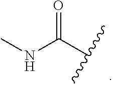 [SMILES: *C(=O)NC]

In some embodiments, each instance of R^{CyA} is independently

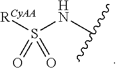

In some embodiments, each instance of R^{CyA} is independently

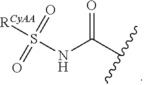

In some embodiments, each instance of each instance of R^{CyA} is

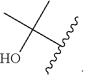 [SMILES: *C(C)(C)O]

In some embodiments, each instance of R^{CyA} is independently

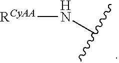

In some embodiments, each instance of R^{CyA} is independently

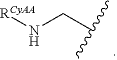

In some embodiments, each instance of R^{CyA} is independently R^{B} substituted by r⁶ instances of R^{CyAC}. In some embodiments, each instance of R^{CyA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein each ring is substituted by r⁶ instances of R^{CyAC}. In some embodiments, each instance of R^{CyA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyA} is independently a 5-6 membered monocyclic heteroaryl ring having 1-2 nitrogen atoms; wherein said ring is substituted by r⁶ instances of R^{CyAC}. In some embodiments, each instance of R^{CyA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}.

In some embodiments, each instance of R^{CyA} is independently

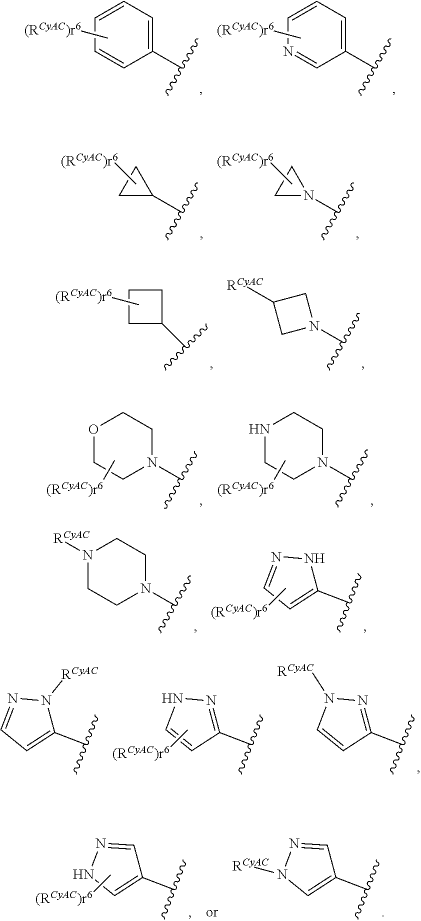

In some embodiments, each instance of R^{CyA} is independently

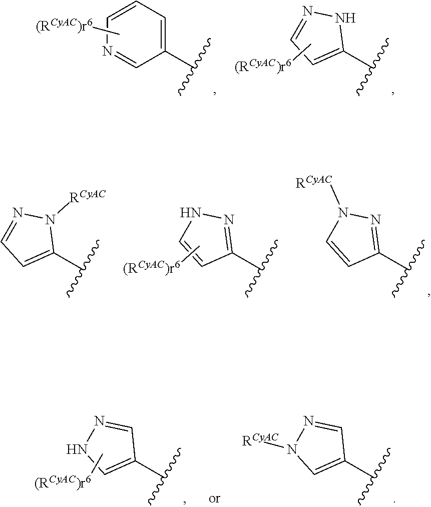

In some embodiments, each instance of R^{CyA} is independently

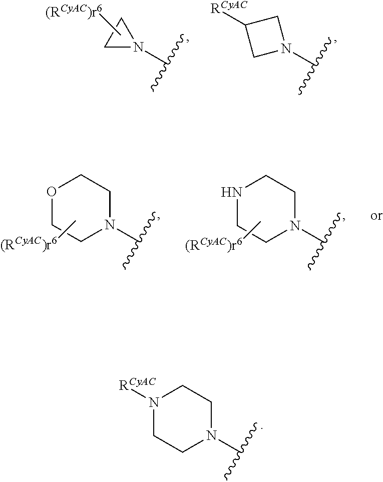

In some embodiments, each instance of R^{CyA} is independently

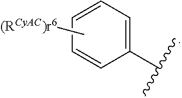

In some embodiments, each instance of R^{CyA} is independently

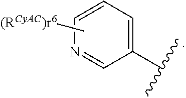

In some embodiments, each instance of R^{CyA} is independently

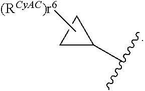

In some embodiments, each instance of R^{CyA} is independently

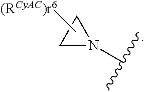

In some embodiments, each instance of R^{CyA} is independently

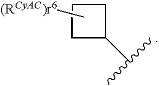

In some embodiments, each instance of R^{CyA} is independently

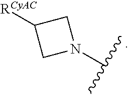

In some embodiments, each instance of R^{CyA} is independently

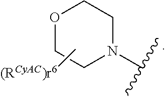

In some embodiments, each instance of R^{CyA} is independently

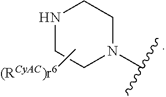

In some embodiments, each instance of R^{CyA} is independently

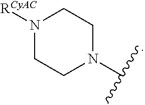

In some embodiments, each instance of R^{CyA} is independently

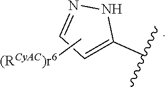

In some embodiments, each instance of R^{CyA} is independently

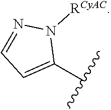

In some embodiments, each instance of R^{CyA} is independently

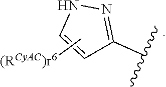

In some embodiments, each instance of R^{CyA} is independently

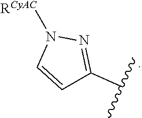

In some embodiments, each instance of R^{CyA} is independently

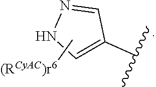

In some embodiments, each instance of R^{CyA} is independently

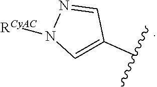

In some embodiments, each instance of R^{CyA} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{CyA} is independently a C₁₋₆ aliphatic that is (i) substituted with 1 or 2 groups independently selected from —O—(C₁₋₆ aliphatic), —OH, —N(C₁₋₆ aliphatic)₂, and —CN, and (ii) optionally substituted with 1, 2, or 3 atoms independently selected from halogen and deuterium. In some embodiments, each instance of R^{CyA} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{CyA} is independently a C₁₋₆ aliphatic substituted with 1 or 2 groups independently selected from —O—(C₁₋₆ aliphatic), —OH, —N(C₁₋₆ aliphatic)₂, and —CN.

In some embodiments, each instance of R^{CyA} is independently a C₁₋₆ aliphatic optionally substituted with 1, 2, or 3 atoms independently selected from halogen and deuterium. In some embodiments, each instance of R^{CyA} is independently a C₁₋₆ aliphatic substituted with 1, 2, or 3 atoms independently selected from halogen and deuterium. In some embodiments, each instance of R^{CyA} is independently a C₁₋₆ aliphatic.

In some embodiments, each instance of R^{CyA} is independently selected from the groups depicted in the compounds in Table 1.

As defined generally above, Cy^{A} is a 5-6 membered saturated or partially unsaturated monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA}.

In some embodiments, Cy^{A} is a 5-6 membered saturated or partially unsaturated monocyclic ring having 0-3 heteroatoms independently selected from nitrogen, oxygen, and sulfur, wherein the monocyclic ring is substituted with n instances of R^{CyA}. In some embodiments, Cy^{A} is a 5-membered saturated or partially unsaturated monocyclic ring having 0-3 heteroatoms independently selected from nitrogen, oxygen, and sulfur, wherein the monocyclic ring is substituted with n instances of R^{CyA}. In some embodiments, Cy^{A} is a 6-membered saturated or partially unsaturated monocyclic ring having 0-3 heteroatoms independently selected from nitrogen, oxygen, and sulfur, wherein the monocyclic ring is substituted with n instances of R^{CyA}.

In some embodiments, Cy^{A} is a 8-10 membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 8-membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 9-membered saturated, partially unsaturated, or aromatic bicyclic ring having 0-4 heteroatoms independently selected from nitrogen, oxygen, and sulfur, wherein each ring is substituted with n instances of R^{CyA}. In some embodiments, Cy^{A} is a 10-membered saturated, partially unsaturated, or aromatic bicyclic ring having 0-4 heteroatoms independently selected from nitrogen, oxygen, and sulfur, wherein each ring is substituted with n instances of R^{CyA}.

In some embodiments, Cy^{A} is a monocyclic or bicyclic ring selected from cyclopentane, cyclohexane, pyrrolidine, pyrazole, thiophene, piperidine, piperazine, benzene, pyridine, pyridazine, pyrimidine, pyrazine, indoline, 1H-indole, [1,2,4]triazolo[4,3-a]pyridine, and quinoline; wherein each ring is substituted with n instances of R^{CyA}.

In some embodiments, Cy^{A} is cyclopentane substituted with n instances of R^{CyA}. In some embodiments, Cy^{A} is cyclohexane substituted with n instances of R^{CyA}. In some embodiments, Cy^{A} is pyrrolidine substituted with n instances of R^{CyA}. In some embodiments, Cy^{A} is pyrazole substituted with n instances of R^{CyA}. In some embodiments, Cy^{A} is thiophene substituted with n instances of R^{CyA}. In some embodiments, Cy^{A} is piperidine substituted with n instances of R^{CyA}. In some embodiments, Cy^{A} is piperazine substituted with n instances of R^{CyA}. In some embodiments, Cy^{A} is benzene substituted with n instances of R^{CyA}. In some embodiments, Cy^{A} is pyridine substituted with n instances of R^{CyA}. In some embodiments, Cy^{A} is pyridazine substituted with n instances of R^{CyA}. In some embodiments, Cy^{A} is pyrimidine substituted with n instances of R^{CyA}. In some embodiments, Cy^{A} is pyrazine substituted with n instances of R^{CyA}. In some embodiments, Cy^{A} is indoline substituted with n instances of R^{CyA}. In some embodiments, Cy^{A} is 1H-indole substituted with n instances of R^{CyA}. In some embodiments, Cy^{A} is [1,2,4]triazolo[4,3-a]pyridine substituted with n instances of R^{CyA}. In some embodiments, Cy^{A} is quinoline substituted with n instances of R^{CyA}.

In some embodiments, Cy^{A} is

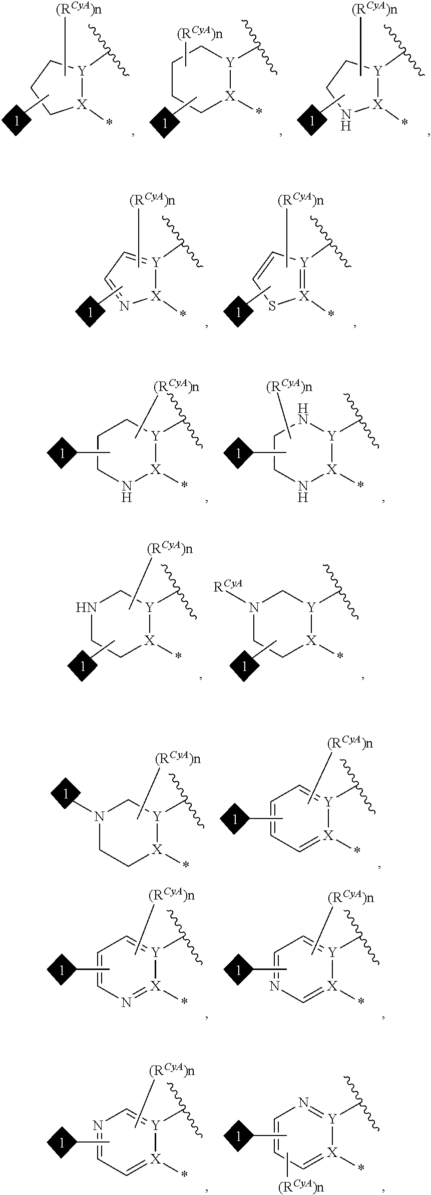

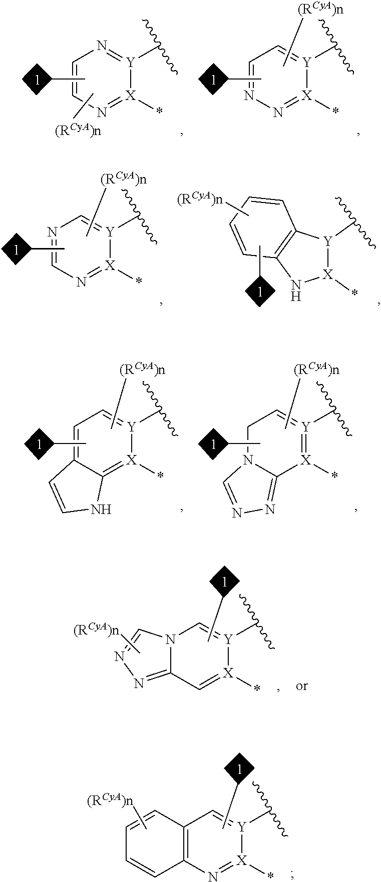

; wherein [symbol] represents a bond to L, [symbol] represents a bond to R¹, and [symbol] represents a bond to R².

In some embodiments, Cy^{A} is

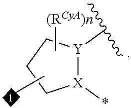

In some embodiments, Cy^{A} is

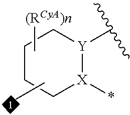

In some embodiments, Cy^{A} is

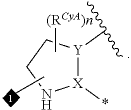

In some embodiments, Cy^{A} is

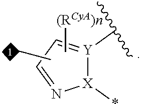

In some embodiments, Cy^{A} is

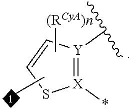

In some embodiments, Cy^{A} is

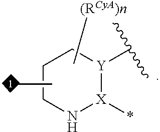

In some embodiments, Cy^{A} is

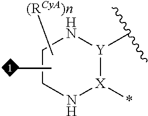

In some embodiments, Cy^{A} is

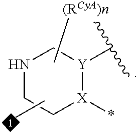

In some embodiments, Cy^{A} is

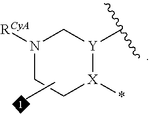

In some embodiments, Cy^{A} is

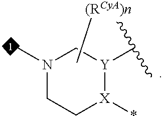

In some embodiments, Cy^{A} is

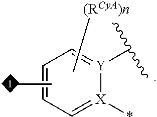

In some embodiments, Cy^{A} is

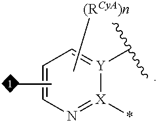

In some embodiments, Cy^{A} is

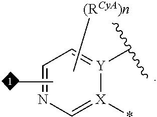

In some embodiments, Cy^{A} is

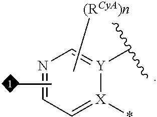

In some embodiments, Cy^{A} is

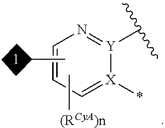

In some embodiments, Cy^{A} is

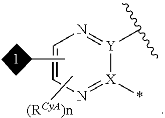

In some embodiments, Cy^{A} is

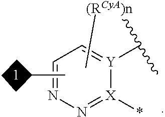

In some embodiments, Cy^{A} is

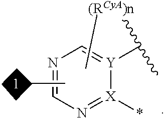

In some embodiments, Cy^{A} is

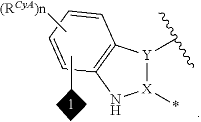

In some embodiments, Cy^{A} is

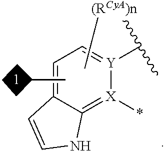

In some embodiments, Cy^{A} is

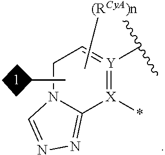

In some embodiments, Cy^{A} is

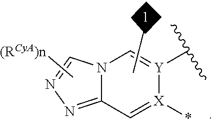

In some embodiments, Cy^{A} is N

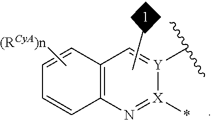

In some embodiments, Cy^{A} is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₄ bivalent saturated or unsaturated, straight or branched hydrocarbon chain.

In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L¹ is a C₁₋₂ bivalent saturated or unsaturated hydrocarbon chain.

In some embodiments, L¹ is —N(H)—, —CH₂—, or a covalent bond. In some embodiments, L¹ is is —N(H)—. In some embodiments, L¹ is —CH₂—. In some embodiments, L₁ is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₄ bivalent saturated or unsaturated, straight or branched hydrocarbon chain.

In some embodiments, L² is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L² is a C₁₋₂ bivalent saturated or unsaturated hydrocarbon chain.

In some embodiments, L² is —N(R)C(O)—, —N(R)C(O)N(R)—, —C(O)N(R)—, —N(R)—, —S(O)₂CH₂—, —CH₂S(O)₂—, or a covalent bond. In some embodiments, L² is —N(H)C(O)—, —N(H)C(O)N(H)—, —C(O)N(H)—, —N(H)—, —S(O)₂CH₂—, —CH₂S(O)₂—, or a covalent bond. In some embodiments, L² is —N(R)C(O)—, —N(R)C(O)N(R)—, —N(R)—, or a covalent bond. In some embodiments, L² is —N(H)C(O)—, —N(H)C(O)N(H)—, —N(H)—, or a covalent bond.

In some embodiments, L² is —N(R)C(O)— or —N(R)C(O)N(R)—. In some embodiments, L² is —N(H)C(O)— or —N(H)C(O)N(H)—. In some embodiments, L² is —N(R)C(O)—. In some embodiments, L² is —N(H)C(O)—. In some embodiments, L² is —N(R)C(O)N(R)—. In some embodiments, L² is —N(H)C(O)N(H)—. In some embodiments, L² is —C(O)N(R)—. In some embodiments, L² is —C(O)N(H)—. In some embodiments, L² is —N(R)—. In some embodiments, L² is —N(H)—. In some embodiments, L² is —S(O)₂CH₂— or —CH₂S(O)₂—. In some embodiments, L² is —S(O)₂CH₂—. In some embodiments, L² is —CH₂S(O)₂—. In some embodiments, L² is a covalent bond. In some embodiments, L² is selected from the groups depicted in the compounds in Table 1.

As defined generally above, L^{X} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a covalent bond. In some embodiments, L^{X} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₄ bivalent saturated or unsaturated, straight or branched hydrocarbon chain.

In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{X} is a C₁₋₂ bivalent saturated or unsaturated hydrocarbon chain. In some embodiments, L^{X} is selected from the groups depicted in the compounds in Table 1.

As defined generally above, L^{Y} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L is a covalent bond. In some embodiments, L^{Y} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₄ bivalent saturated or unsaturated, straight or branched hydrocarbon chain.

In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Y} is a C₁₋₂ bivalent saturated or unsaturated hydrocarbon chain.

In some embodiments, L^{Y} is —C(O)N(R)—, —C(O)N(R)CH₂—, or a covalent bond. In some embodiments, L^{Y} is —C(O)N(H)—, —C(O)N(H)CH₂—, or a covalent bond. In some embodiments, L^{Y} is —C(O)N(H)— or —C(O)N(H)CH₂—. In some embodiments, L^{Y} is —C(O)N(H)—. In some embodiments, L^{Y} is —C(O)N(H)CH₂—. In some embodiments, L^{Y} is selected from the groups depicted in the compounds in Table 1.

As defined generally above, L^{CyA} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a covalent bond. In some embodiments, L^{CyA} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a C₁₋₄ bivalent saturated or unsaturated, straight or branched hydrocarbon chain.

In some embodiments, L^{CyA} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{CyA} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{CyA} is a C₁₋₂ bivalent saturated or unsaturated hydrocarbon chain.

In some embodiments, L^{CyA} is —C(O)N(R)—, —C(O)N(R)CH₂—, or a covalent bond. In some embodiments, L^{CyA} is —C(O)N(H)—, —C(O)N(H)CH₂—, or a covalent bond. In some embodiments, L^{CyA} is —C(O)N(H)— or —C(O)N(H)CH₂—. In some embodiments, L^{CyA} is —C(O)N(H)—. In some embodiments, L^{CyA} is —C(O)N(H)CH₂—. In some embodiments, L^{CyA} is selected from the groups depicted in the compounds in Table 1.

As defined generally above, R^{1A} is R^{A} or R^{B} substituted by r instances of R^{1C}. In some embodiments, R^{1A} is R^{A}. In some embodiments, R^{1A} is R substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{1A} is substituted by r¹ instances of R^{1C}.

In some embodiments, R^{1A} is phenyl substituted by r¹ instances of R^{1C}. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of R^{1C}. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{1A} is substituted by r¹ instances of R^{1C}.

In some embodiments, R^{1A} is phenyl substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{1A} is phenyl substituted by 1-3 instances of R^{1C}. In some embodiments, R^{1A} is phenyl substituted by 2 instances of R^{1C}. In some embodiments, R^{1A} is phenyl substituted by 1 instance of R^{1C}.

In some embodiments, R^{1A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{1A} is phenyl substituted by 1-3 instances of a group independently selected from halogen and C₁₋₃ aliphatic optionally substituted with 1-3 halogen. In some embodiments, R^{1A} is phenyl substituted by 1-3 instances of a group independently selected from fluorine, chlorine, —CH₃, —CHF₂, and —CF₃.

In some embodiments, R^{1A} is phenyl substituted by 2 instances of a group independently selected from halogen, —CN, —O-(optionally substituted C₁₋₆ aliphatic), and an optionally substituted C₁₋₆ aliphatic. In some embodiments, R^{1A is} phenyl substituted by 2 instances of a group independently selected from halogen and C₁₋₃ aliphatic optionally substituted with 1-3 halogen. In some embodiments, R^{1A is} phenyl substituted by 2 instances of a group independently selected from fluorine, chlorine, —CH₃, —CHF₂, and —CF₃.

In some embodiments, R^{1A} is phenyl substituted by one group selected from halogen, —CN, —O-(optionally substituted C₁₋₆ aliphatic), and an optionally substituted C₁₋₆ aliphatic. In some embodiments, R^{1A} is phenyl substituted by one halogen or C₁₋₃ aliphatic group optionally substituted with 1-3 halogen. In some embodiments, R^{1A} is phenyl substituted by one fluorine, chlorine, —CH₃, —CHF₂, or —CF₃.

In some embodiments, R^{1A} is

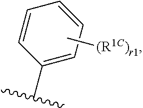

wherein R^{1C} and r¹ are as defined in the embodiments and classes and subclasses herein. In some embodiments, R^{1A} is

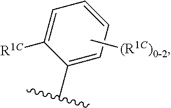

wherein R^{1C} is as defined in the embodiments and classes and subclasses herein. In some embodiments, R^{1A} is

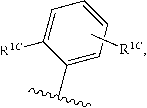

wherein R^{1C} is as defined in the embodiments and classes and subclasses herein. In some embodiments, R^{1A} is

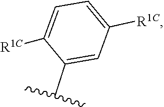

wherein R^{1C} is as defined in the embodiments and classes and subclasses herein. In some embodiments, R^{1A} is

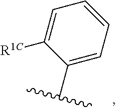

wherein R^{1C} is as defined in the embodiments and classes and subclasses herein.

In some embodiments, R^{1A} is

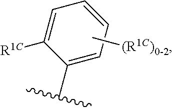

wherein each instance of R^{1C} is independently halogen, —CN, —O-(optionally substituted C₁₋₆ aliphatic), or an optionally substituted C₁₋₆ aliphatic. In some embodiments, R^{1A} is

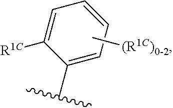

wherein each instance of R^{1C} is independently halogen or C₁₋₃ aliphatic optionally substituted with 1-3 halogen. In some embodiments, R^{1A} is

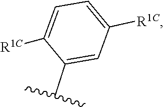

wherein each instance of R^{1C} is independently halogen or C₁₋₃ aliphatic optionally substituted with 1-3 halogen. In some embodiments, R^{1A} is

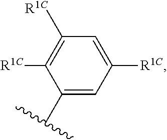

wherein each instance of R^{1C} is independently halogen or C₁₋₃ aliphatic optionally substituted with 1-3 halogen. In some embodiments, R^{1A} is

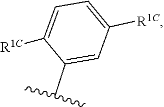

wherein each instance of R^{1C} is independently fluorine, chlorine, —CH₃, —CHF₂, or —CF₃. In some embodiments, R^{1A} is

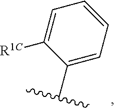

wherein R^{1C} is halogen or C₁₋₃ aliphatic optionally substituted with 1-3 halogen.

In some embodiments, R^{1A} is

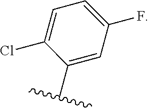 [SMILES: *c1cc(F)ccc1Cl]

In some embodiments, R^{1A} is

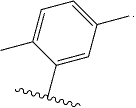

In some embodiments, R^{1A} is

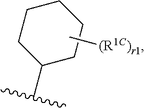

wherein R^{1C} and r¹ are as defined in the embodiments and classes and subclasses herein. In some embodiments, R^{1A} is

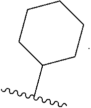 [SMILES: *C1CCCCC1]

In some embodiments, R^{1A} is

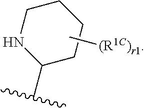

In some embodiments, R^{1A} is

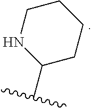 [SMILES: *C1CCCCN1]

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is oxo. In some embodiments, R^{1A} is halogen. In some embodiments, R^{1A} is —CN. In some embodiments, R^{1A} is —NO₂. In some embodiments, R^{1A} is —OR. In some embodiments, R^{1A} is —SR. In some embodiments, R^{1A} is —NR₂. In some embodiments, R^{1A} is —S(O)₂R. In some embodiments, R^{1A} is —S(O)₂NR₂. In some embodiments, R^{1A} is —S(O)₂F. In some embodiments, R^{1A} is —S(O)R. In some embodiments, R^{1A} is —S(O)NR₂. In some embodiments, R^{1A} is —S(O)(NR)R. In some embodiments, R^{1A} is —C(O)R. In some embodiments, R^{1A} is —C(O)OR. In some embodiments, R^{1A} is —C(O)NR₂. In some embodiments, R^{1A} is —C(O)N(R)OR. In some embodiments, R^{1A} is —OC(O)R. In some embodiments, R^{1A} is —OC(O)NR₂. In some embodiments, R^{1A} is —N(R)C(O)OR. In some embodiments, R^{1A} is —N(R)C(O)R. In some embodiments, R^{1A} is —N(R)C(O)NR₂. In some embodiments, R^{1A} is —N(R)C(NR)NR₂. In some embodiments, R^{1A} is —N(R)S(O)₂NR₂. In some embodiments, R^{1A} is —N(R)S(O)₂R. In some embodiments, R^{1A} is —P(O)R₂. In some embodiments, R^{1A} is —P(O)(R)OR. In some embodiments, R^{1A} is —B(OR)₂. In some embodiments, R^{1A} is deuterium.

In some embodiments, R^{1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is halogen, —CN, or —NO₂. In some embodiments, R^{1A} is —OR, —SR, or —NR₂. In some embodiments, R^{1A} is —S(O)₂R, —S(O)₂NR₂, —S(O)₂F, —S(O)R, —S(O)NR₂, or —S(O)(NR)R. In some embodiments, R^{1A} is —C(O)R, —C(O)OR, —C(O)NR₂, or —C(O)N(R)OR. In some embodiments, R^{1A} is —OC(O)R or —OC(O)NR₂. In some embodiments, R^{1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{1A} is —P(O)R₂ or —P(O)(R)OR.

In some embodiments, R^{1A} is —OR, —OC(O)R, or —OC(O)NR₂. In some embodiments, R^{1A} is —SR, —S(O)₂R, —S(O)₂NR₂, —S(O)₂F, —S(O)R, —S(O)NR₂, or —S(O)(NR)R. In some embodiments, R^{1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{1A} is —S(O)₂R, —S(O)₂NR₂, or —S(O)₂F. In some embodiments, R^{1A} is —S(O)R, —S(O)NR₂, or —S(O)(NR)R. In some embodiments, R^{1A} is —SR, —S(O)₂R, or —S(O)R. In some embodiments, R^{1A} is —S(O)₂NR₂, —S(O)NR₂, or —S(O)(NR)R. In some embodiments, R^{1A} is —S(O)₂NR₂ or —S(O)NR₂. In some embodiments, R^{1A} is —SR, —S(O)₂R, —S(O)₂NR₂, or —S(O)R.

In some embodiments, R^{1A} is —N(R)C(O)OR, —N(R)C(O)R, or —N(R)C(O)NR₂. In some embodiments, R^{1A} is —N(R)S(O)₂NR₂ or —N(R)S(O)₂R. In some embodiments, R^{1A} is —N(R)C(O)OR or —N(R)C(O)R. In some embodiments, R^{1A} is —N(R)C(O)NR₂ or —N(R)S(O)₂NR₂. In some embodiments, R^{1A} is —N(R)C(O)OR, —N(R)C(O)R, or —N(R)S(O)₂R.

In some embodiments, R^{1A} is —NR₂, —N(R)C(O)OR, —N(R)C(O)R, or —N(R)C(O)NR₂. In some embodiments, R^{1A} is —NR₂, —N(R)C(O)OR, or —N(R)C(O)R. In some embodiments, R^{1A} is —NR₂, —N(R)C(O)OR, —N(R)C(O)R, or —N(R)S(O)₂R.

In some embodiments, R^{1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain substituted by r¹ instances of R^{1C} In some embodiments, R^{1A} is phenyl substituted by r¹ instances of R^{1C}. In some embodiments, R^{1A} is naphthyl substituted by r¹ instances of R^{1C}. In some embodiments, R^{1A} is a 5-6 membered monocyclic heteroaryl ring having 1-4 heteroatoms independently selected from nitrogen, oxygen, and sulfur; wherein said ring is substituted by r¹ instances of R^{1C}. In some embodiments, R^{1A} is an 8-10 membered bicyclic heteroaryl ring having 1-4 heteroatoms independently selected from nitrogen, oxygen, and sulfur; wherein said ring is substituted by r¹ instances of R^{1C}. In some embodiments, R^{1A} is a 3-7 membered saturated or partially unsaturated monocyclic carbocyclic ring substituted by r¹ instances of R^{1C}. In some embodiments, R^{1A} is a 5-12 membered saturated or partially unsaturated bicyclic carbocyclic ring substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; wherein said ring is substituted by r¹ instances of R^{1C}. In some embodiments, R^{1A} is a 7-12 membered saturated or partially unsaturated bicyclic heterocyclic ring having 1-4 heteroatoms independently selected from nitrogen, oxygen, and sulfur; wherein said ring is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or naphthyl; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 5-12 membered saturated or partially unsaturated bicyclic carbocyclic ring; each of which is substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 5-6 membered monocyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 3-7 membered saturated or partially unsaturated monocyclic carbocyclic ring; each of which is substituted by r¹ instances of R^{1C} In some embodiments, R^{1A} is naphthyl or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}. In some embodiments, R^{1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}.

In some embodiments, R^{1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r¹ instances of R^{1C}. In some embodiments, R^{1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is a C₁₋₆ aliphatic chain, phenyl, or a 3-7 membered saturated or partially unsaturated monocyclic carbocyclic ring; each of which is substituted by r¹ instances of R^{1C}.

In some embodiments, R^{1A} is selected from the groups depicted in the compounds in Table 1.

As defined generally above, R^{2A} is R^{A} or R^{B} substituted by r² instances of R^{2C}. In some embodiments, R^{2A} is R^{A}. In some embodiments, R^{2A} is R^{B} substituted by r² instances of R^{2C}.

In some embodiments, R^{2A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by r² instances of R^{2C}. In some embodiments, R^{2A} is phenyl substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{2A} is phenyl substituted by 1-3 instances of a group independently selected from halogen and C₁₋₃ aliphatic optionally substituted with 1-3 halogen. In some embodiments, R^{2A} is phenyl substituted by 1-3 instances of a group independently selected from fluorine, chlorine, —CH₃, —CHF₂, and —CF₃.

In some embodiments, R^{2A} is phenyl substituted by 2 instances of a group independently selected from halogen, —CN, —O-(optionally substituted C₁₋₆ aliphatic), and an optionally substituted C₁₋₆ aliphatic. In some embodiments, R^{2A} is phenyl substituted by 2 instances of a group independently selected from halogen and C₁₋₃ aliphatic optionally substituted with 1-3 halogen. In some embodiments, R^{2A} is phenyl substituted by 2 instances of a group independently selected from fluorine, chlorine, —CH₃, —CHF₂, and —CF₃.

In some embodiments, R^{2A} is an 8-10 membered bicyclic heteroaryl ring having 1-4 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-2 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen, —CN, —O-(optionally substituted C₁₋₆ aliphatic), and an optionally substituted C₁₋₆ aliphatic. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen and C₁₋₃ aliphatic optionally substituted with 1-3 halogen. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from fluorine, chlorine, —CH₃, —CHF₂, and —CF₃.

In some embodiments, R^{2A} is:

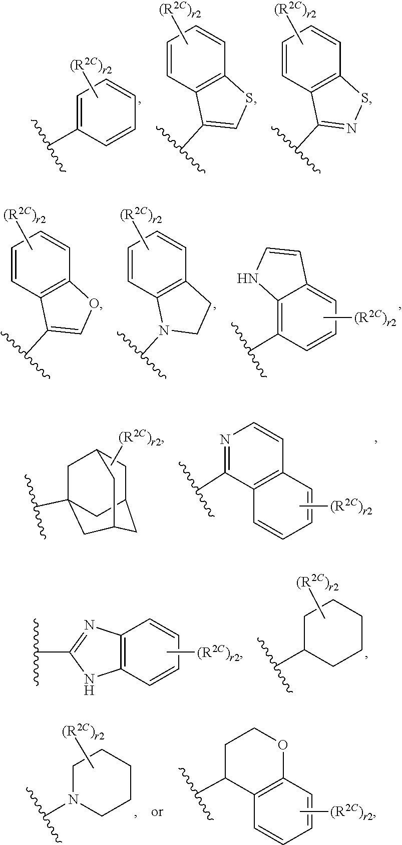

wherein R^{2C} and r² are as defined in the embodiments and classes and subclasses herein. In some embodiments, R^{2A} is

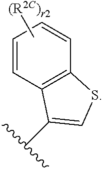

In some embodiments, R^{2A} is

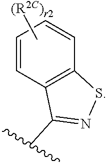

In some embodiments, R^{2A} is

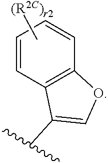

In some embodiments, R^{2A} is

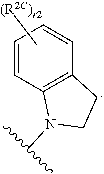

In some embodiments, R^{2A} is

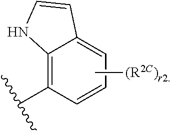

In some embodiments, R^{2A} is

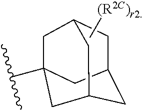

In some embodiments, R^{2A} is

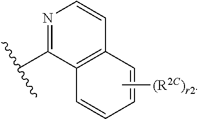

In some embodiments, R^{2A} is

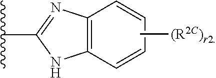

In some embodiments, R^{2A} is

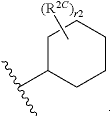

In some embodiments, R^{2A} is

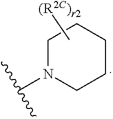

In some embodiments, R^{2A} is

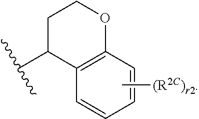

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is oxo. In some embodiments, R^{2A} is halogen. In some embodiments, R^{2A} is —CN. In some embodiments, R^{2A} is —NO₂. In some embodiments, R^{2A} is —OR. In some embodiments, R^{2A} is —SR. In some embodiments, R^{2A} is —NR₂. In some embodiments, R^{2A} is —S(O)₂R. In some embodiments, R^{2A} is —S(O)₂NR₂. In some embodiments, R^{2A} is —S(O)₂F. In some embodiments, R^{2A} is —S(O)R. In some embodiments, R^{2A} is —S(O)NR₂. In some embodiments, R^{2A} is —S(O)(NR)R. In some embodiments, R^{2A} is —C(O)R. In some embodiments, R^{2A} is —C(O)OR. In some embodiments, R^{2A} is —C(O)NR₂. In some embodiments, R^{2A} is —C(O)N(R)OR. In some embodiments, R^{2A} is —OC(O)R. In some embodiments, R^{2A} is —OC(O)NR₂. In some embodiments, R^{2A} is —N(R)C(O)OR. In some embodiments, R^{2A} is —N(R)C(O)R. In some embodiments, R^{2A} is —N(R)C(O)NR₂. In some embodiments, R^{2A} is —N(R)C(NR)NR₂. In some embodiments, R^{2A} is —N(R)S(O)₂NR₂. In some embodiments, R^{2A} is —N(R)S(O)₂R. In some embodiments, R^{2A} is —P(O)R₂. In some embodiments, R^{2A} is —P(O)(R)OR. In some embodiments, R^{2A} is —B(OR)₂. In some embodiments, R^{2A} is deuterium.

In some embodiments, R^{2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is halogen, —CN, or —NO₂. In some embodiments, R^{2A} is —OR, —SR, or —NR₂. In some embodiments, R^{2A} is —S(O)₂R, —S(O)₂NR₂, —S(O)₂F, —S(O)R, —S(O)NR₂, or —S(O)(NR)R. In some embodiments, R^{2A} is —C(O)R, —C(O)OR, —C(O)NR₂, or —C(O)N(R)OR. In some embodiments, R^{2A} is —OC(O)R or —OC(O)NR₂. In some embodiments, R^{2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{2A} is —P(O)R₂ or —P(O)(R)OR.

In some embodiments, R^{2A} is —OR, —OC(O)R, or —OC(O)NR₂. In some embodiments, R^{2A} is —SR, —S(O)₂R, —S(O)₂NR₂, —S(O)₂F, —S(O)R, —S(O)NR₂, or —S(O)(NR)R. In some embodiments, R^{2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{2A} is —S(O)₂R, —S(O)₂NR₂, or —S(O)₂F. In some embodiments, R^{2A} is —S(O)R, —S(O)NR₂, or —S(O)(NR)R. In some embodiments, R^{2A} is —SR, —S(O)₂R, or —S(O)R. In some embodiments, R^{2A} is —S(O)₂NR₂, —S(O)NR₂, or —S(O)(NR)R. In some embodiments, R^{2A} is —S(O)₂NR₂ or —S(O)NR₂. In some embodiments, R^{2A} is —SR, —S(O)₂R, —S(O)₂NR₂, or —S(O)R.

In some embodiments, R^{2A} is —N(R)C(O)OR, —N(R)C(O)R, or —N(R)C(O)NR₂. In some embodiments, R^{2A} is —N(R)S(O)₂NR₂ or —N(R)S(O)₂R. In some embodiments, R^{2A} is —N(R)C(O)OR or —N(R)C(O)R. In some embodiments, R^{2A} is —N(R)C(O)NR₂ or —N(R)S(O)₂NR₂. In some embodiments, R^{2A} is —N(R)C(O)OR, —N(R)C(O)R, or —N(R)S(O)₂R.

In some embodiments, R^{2A} is —NR₂, —N(R)C(O)OR, —N(R)C(O)R, or —N(R)C(O)NR₂. In some embodiments, R^{2A} is —NR₂, —N(R)C(O)OR, or —N(R)C(O)R. In some embodiments, R^{2A} is —NR₂, —N(R)C(O)OR, —N(R)C(O)R, or —N(R)S(O)₂R.

In some embodiments, R^{2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain substituted by r² instances of R^{2C}. In some embodiments, R^{2A} is phenyl substituted by r² instances of R^{2C}. In some embodiments, R^{2A} is naphthyl substituted by r² instances of R^{2C}. In some embodiments, R^{2A} is cubanyl substituted by r² instances of R^{2C}. In some embodiments, R^{2A} is adamantyl substituted by r² instances of R^{2C}. In some embodiments, R^{2A} is a 5-6 membered monocyclic heteroaryl ring having 1-4 heteroatoms independently selected from nitrogen, oxygen, and sulfur; wherein said ring is substituted by r² instances of R^{2C}. In some embodiments, R^{2A} is an 8-10 membered bicyclic heteroaryl ring having 1-4 heteroatoms independently selected from nitrogen, oxygen, and sulfur; wherein said ring is substituted by r² instances of R^{2C}. In some embodiments, R^{2A} is a 3-7 membered saturated or partially unsaturated monocyclic carbocyclic ring substituted by r² instances of R^{2C}. In some embodiments, R^{2A} is a 5-12 membered saturated or partially unsaturated bicyclic carbocyclic ring substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}. In some embodiments, R^{2A} is a 7-12 membered saturated or partially unsaturated bicyclic heterocyclic ring having 1-4 heteroatoms independently selected from nitrogen, oxygen, and sulfur; wherein said ring is substituted by r² instances of R^{2C}.

In some embodiments, R^{2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or naphthyl; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 5-12 membered saturated or partially unsaturated bicyclic carbocyclic ring; each of which is substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 5-6 membered monocyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 3-7 membered saturated or partially unsaturated monocyclic carbocyclic ring; each of which is substituted by r² instances of R^{2C}. In some embodiments, R^{2A} is naphthyl; cubanyl; adamantyl; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}. In some embodiments, R^{2A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}.

In some embodiments, R^{2A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by r² instances of R^{2C}. In some embodiments, R^{2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is a C₁₋₆ aliphatic chain, phenyl, or a 3-7 membered saturated or partially unsaturated monocyclic carbocyclic ring; each of which is substituted by r² instances of R^{2C}.

In some embodiments, R^{2A} is selected from the groups depicted in the compounds in Table 1.

As defined generally above, R^{XA} is R^{A} or R^{B} substituted by r³ instances of R^{XC}. In some embodiments, R^{XA} is R^{A}. In some embodiments, R^{XA} is R^{B} substituted by r³ instances of R^{XC}.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is oxo. In some embodiments, R^{XA} is halogen. In some embodiments, R^{XA} is —CN. In some embodiments, R^{XA} is —NO₂. In some embodiments, R^{XA} is —OR. In some embodiments, R^{XA} is —SR. In some embodiments, R^{XA} is —NR₂. In some embodiments, R^{XA} is —S(O)₂R. In some embodiments, R^{XA} is —S(O)₂NR₂. In some embodiments, R^{XA} is —S(O)₂F. In some embodiments, R^{XA} is —S(O)R. In some embodiments, R^{XA} is —S(O)NR₂. In some embodiments, R^{XA} is —S(O)(NR)R. In some embodiments, R^{XA} is —C(O)R. In some embodiments, R^{XA} is —C(O)OR. In some embodiments, R^{XA} is —C(O)NR₂. In some embodiments, R^{XA} is —C(O)N(R)OR. In some embodiments, R^{XA} is —OC(O)R. In some embodiments, R^{XA} is —OC(O)NR₂. In some embodiments, R^{XA} is —N(R)C(O)OR. In some embodiments, R^{XA} is —N(R)C(O)R. In some embodiments, R^{XA} is —N(R)C(O)NR₂. In some embodiments, R^{XA} is —N(R)C(NR)NR₂. In some embodiments, R^{XA} is —N(R)S(O)₂NR₂. In some embodiments, R^{XA} is —N(R)S(O)₂R. In some embodiments, R^{XA} is —P(O)R₂. In some embodiments, R^{XA} is —P(O)(R)OR. In some embodiments, R^{XA} is —B(OR)₂. In some embodiments, R^{XA} is deuterium.

In some embodiments, R^{XA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is halogen, —CN, or —NO₂. In some embodiments, R^{XA} is —OR, —SR, or —NR₂. In some embodiments, R^{XA} is —S(O)₂R, —S(O)₂NR₂, —S(O)₂F, —S(O)R, —S(O)NR₂, or —S(O)(NR)R. In some embodiments, R^{XA} is —C(O)R, —C(O)OR, —C(O)NR₂, or —C(O)N(R)OR. In some embodiments, R^{XA} is —OC(O)R or —OC(O)NR₂. In some embodiments, R^{XA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{XA} is —P(O)R₂ or —P(O)(R)OR.

In some embodiments, R^{XA} is —OR, —OC(O)R, or —OC(O)NR₂. In some embodiments, R^{XA} is —SR, —S(O)₂R, —S(O)₂NR₂, —S(O)₂F, —S(O)R, —S(O)NR₂, or —S(O)(NR)R. In some embodiments, R^{XA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{XA} is —S(O)₂R, —S(O)₂NR₂, or —S(O)₂F. In some embodiments, R^{XA} is —S(O)R, —S(O)NR₂, or —S(O)(NR)R. In some embodiments, R^{XA} is —SR, —S(O)₂R, or —S(O)R. In some embodiments, R^{XA} is —S(O)₂NR₂, —S(O)NR₂, or —S(O)(NR)R.

In some embodiments, R^{XA} is —S(O)₂NR₂ or —S(O)NR₂. In some embodiments, R^{XA} is —SR, —S(O)₂R, —S(O)₂NR₂, or —S(O)R.

In some embodiments, R^{XA} is —N(R)C(O)OR, —N(R)C(O)R, or —N(R)C(O)NR₂. In some embodiments, R^{XA} is —N(R)S(O)₂NR₂ or —N(R)S(O)₂R. In some embodiments, R^{XA} is —N(R)C(O)OR or —N(R)C(O)R. In some embodiments, R^{XA} is —N(R)C(O)NR₂ or —N(R)S(O)₂NR₂. In some embodiments, R^{XA} is —N(R)C(O)OR, —N(R)C(O)R, or —N(R)S(O)₂R.

In some embodiments, R^{XA} is —NR₂, —N(R)C(O)OR, —N(R)C(O)R, or —N(R)C(O)NR₂. In some embodiments, R^{XA} is —NR₂, —N(R)C(O)OR, or —N(R)C(O)R. In some embodiments, R^{XA} is —NR₂, —N(R)C(O)OR, —N(R)C(O)R, or —N(R)S(O)₂R.

In some embodiments, R^{XA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is a C₁₋₆ aliphatic chain substituted by r³ instances of R^{XC} In some embodiments, R^{XA} is phenyl substituted by r³ instances of R^{XC}. In some embodiments, R^{XA} is naphthyl substituted by r³ instances of R^{XC}. In some embodiments, R^{XA} is a 5-6 membered monocyclic heteroaryl ring having 1-4 heteroatoms independently selected from nitrogen, oxygen, and sulfur; wherein said ring is substituted by r³ instances of R^{XC}. In some embodiments, R^{XA} is an 8-10 membered bicyclic heteroaryl ring having 1-4 heteroatoms independently selected from nitrogen, oxygen, and sulfur; wherein said ring is substituted by r³ instances of R^{XC}. In some embodiments, R^{XA} is a 3-7 membered saturated or partially unsaturated monocyclic carbocyclic ring substituted by r³ instances of R^{XC}. In some embodiments, R^{XA} is a 5-12 membered saturated or partially unsaturated bicyclic carbocyclic ring substituted by r³ instances of R^{XC}. In some embodiments, R^{X} is a 3-7 membered saturated or partially unsaturated monocyclic heterocyclic ring having 1-2 heteroatoms independently selected from nitrogen, oxygen, and sulfur; wherein said ring is substituted by r³ instances of R^{XC}. In some embodiments, R^{XA} is a 7-12 membered saturated or partially unsaturated bicyclic heterocyclic ring having 1-4 heteroatoms independently selected from nitrogen, oxygen, and sulfur; wherein said ring is substituted by r³ instances of R^{XC}.

In some embodiments, R^{X}A is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{XC}. In some embodiments, R^{A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, Rx is phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{XC}. In some embodiments, Rx is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or naphthyl; each of which is substituted by r³ instances of R^{XC}. In some embodiments, Rx is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{XC}. In some embodiments, R^{A} is a 3-7 membered saturated or partially unsaturated monocyclic carbocyclic ring or a 5-12 membered saturated or partially unsaturated bicyclic carbocyclic ring; each of which is substituted by r³ instances of R^{XC}. In some embodiments, R^{A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or a 5-6 membered monocyclic heteroaryl ring having 1-4 heteroatoms independently selected from nitrogen, oxygen, and sulfur; each of which is substituted by r³ instances of R^{XC}. In some embodiments, R^{XA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is naphthyl or an 8-10 membered bicyclic heteroaryl ring having 1-4 heteroatoms independently selected from nitrogen, oxygen, and sulfur; each of which is substituted by r³ instances of R^{XC}. In some embodiments, R^{XA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is phenyl or a 3-7 membered saturated or partially unsaturated monocyclic carbocyclic ring; each of which is substituted by r³ instances of R^{XC} In some embodiments, R^{XA} is naphthyl or a 5-12 membered saturated or partially unsaturated bicyclic carbocyclic ring; each of which is substituted by r³ instances of R^{XC}. In some embodiments, R^{XA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}.

In some embodiments, R^{XA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{XC}. In some embodiments, R^{XA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{XC}. In some embodiments, R^{XA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{XC}.

In some embodiments, R^{XA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r³ instances of R^{XC}. In some embodiments, R^{XA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r³ instances of R^{XC}. In some embodiments, R^{XA} is a C₁₋₆ aliphatic chain, phenyl, or a 3-7 membered saturated or partially unsaturated monocyclic carbocyclic ring; each of which is substituted by r³ instances of R^{XC}.

In some embodiments, R^{XA} is selected from the groups depicted in the compounds in Table 1.

As defined generally above, R^{YA} is R^{A} or R^{B} substituted by r⁴ instances of R^{YC}. In some embodiments, R^{YA} is R^{A}. In some embodiments, R^{YA} is R^{B} substituted by r⁴ instances of R^{YC}.

In some embodiments, R^{YA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{YA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{YA} is oxo. In some embodiments, R^{YA} is halogen. In some embodiments, R^{YA} is —CN. In some embodiments, R^{YA} is —NO₂. In some embodiments, R^{YA} is —OR. In some embodiments, R^{YA} is —SR. In some embodiments, R^{YA} is —NR₂. In some embodiments, R^{YA} is —S(O)₂R. In some embodiments, R^{YA} is —S(O)₂NR₂. In some embodiments, R^{YA} is —S(O)₂F. In some embodiments, R^{YA} is —S(O)R. In some embodiments, R^{YA} is —S(O)NR₂. In some embodiments, R^{YA} is —S(O)(NR)R. In some embodiments, R^{YA} is —C(O)R. In some embodiments, R^{YA} is —C(O)OR. In some embodiments, R^{YA} is —C(O)NR₂. In some embodiments, R^{YA} is —C(O)N(R)OR. In some embodiments, R^{YA} is —OC(O)R. In some embodiments, R^{YA} is —OC(O)NR₂. In some embodiments, R^{YA} is —N(R)C(O)OR. In some embodiments, R^{YA} is —N(R)C(O)R. In some embodiments, R^{YA} is —N(R)C(O)NR₂. In some embodiments, R^{YA} is —N(R)C(NR)NR₂. In some embodiments, R^{YA} is —N(R)S(O)₂NR₂. In some embodiments, R^{YA} is —N(R)S(O)₂R. In some embodiments, R^{YA} is —P(O)R₂. In some embodiments, R^{YA} is —P(O)(R)OR. In some embodiments, R^{YA} is —B(OR)₂. In some embodiments, R^{YA} is deuterium.

In some embodiments, R^{YA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{YA} is halogen, —CN, or —NO₂. In some embodiments, R^{YA} is —OR, —SR, or —NR₂. In some embodiments, R^{YA} is —S(O)₂R, —S(O)₂NR₂, —S(O)₂F, —S(O)R, —S(O)NR₂, or —S(O)(NR)R. In some embodiments, R^{YA} is —C(O)R, —C(O)OR, —C(O)NR₂, or —C(O)N(R)OR. In some embodiments, R^{YA} is —OC(O)R or —OC(O)NR₂. In some embodiments, R^{YA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{YA} is —P(O)R₂ or —P(O)(R)OR.

In some embodiments, R^{YA} is —OR, —OC(O)R, or —OC(O)NR₂. In some embodiments, R^{YA} is —SR, —S(O)₂R, —S(O)₂NR₂, —S(O)₂F, —S(O)R, —S(O)NR₂, or —S(O)(NR)R. In some embodiments, R^{YA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{YA} is —S(O)₂R, —S(O)₂NR₂, or —S(O)₂F. In some embodiments, R^{YA} is —S(O)R, —S(O)NR₂, or —S(O)(NR)R. In some embodiments, R^{YA} is —SR, —S(O)₂R, or —S(O)R. In some embodiments, R^{YA} is —S(O)₂NR₂, —S(O)NR₂, or —S(O)(NR)R.

In some embodiments, R^{YA} is —S(O)₂NR₂ or —S(O)NR₂. In some embodiments, R^{YA} is —SR, —S(O)₂R, —S(O)₂NR₂, or —S(O)R.

In some embodiments, R^{YA} is —N(R)C(O)OR, —N(R)C(O)R, or —N(R)C(O)NR₂. In some embodiments, R^{YA} is —N(R)S(O)₂NR₂ or —N(R)S(O)₂R. In some embodiments, R^{YA} is —N(R)C(O)OR or —N(R)C(O)R. In some embodiments, R^{YA} is —N(R)C(O)NR₂ or —N(R)S(O)₂NR₂. In some embodiments, R^{YA} is —N(R)C(O)OR, —N(R)C(O)R, or —N(R)S(O)₂R.

In some embodiments, R^{YA} is —NR₂, —N(R)C(O)OR, —N(R)C(O)R, or —N(R)C(O)NR₂. In some embodiments, R^{YA} is —NR₂, —N(R)C(O)OR, or —N(R)C(O)R. In some embodiments, R^{YA} is —NR₂, —N(R)C(O)OR, —N(R)C(O)R, or —N(R)S(O)₂R.

In some embodiments, R^{YA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is a C₁₋₆ aliphatic chain substituted by r⁴ instances of R^{YC}. In some embodiments, R^{YA} is phenyl substituted by r⁴ instances of R^{YC}. In some embodiments, R^{YA} is naphthyl substituted by r⁴ instances of R^{YC}. In some embodiments, R^{YA} is a 5-6 membered monocyclic heteroaryl ring having 1-4 heteroatoms independently selected from nitrogen, oxygen, and sulfur; wherein said ring is substituted by r⁴ instances of R^{YC}. In some embodiments, R^{YA} is an 8-10 membered bicyclic heteroaryl ring having 1-4 heteroatoms independently selected from nitrogen, oxygen, and sulfur; wherein said ring is substituted by r⁴ instances of R^{YC}. In some embodiments, R^{YA} is a 3-7 membered saturated or partially unsaturated monocyclic carbocyclic ring substituted by r⁴ instances of R^{YC}. In some embodiments, R^{YA} is a 5-12 membered saturated or partially unsaturated bicyclic carbocyclic ring substituted by r⁴ instances of R^{YC}. In some embodiments, R^{YA} is a 3-7 membered saturated or partially unsaturated monocyclic heterocyclic ring having 1-2 heteroatoms independently selected from nitrogen, oxygen, and sulfur; wherein said ring is substituted by r⁴ instances of R^{YC}. In some embodiments, R^{YA} is a 7-12 membered saturated or partially unsaturated bicyclic heterocyclic ring having 1-4 heteroatoms independently selected from nitrogen, oxygen, and sulfur; wherein said ring is substituted by r⁴ instances of R^{YC}.

In some embodiments, R^{YA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or naphthyl; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring or a 5-12 membered saturated or partially unsaturated bicyclic carbocyclic ring; each of which is substituted by r⁴ instances of R^{YC}. In some embodiments, R^{YA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or a 5-6 membered monocyclic heteroaryl ring having 1-4 heteroatoms independently selected from nitrogen, oxygen, and sulfur; each of which is substituted by r⁴ instances of R^{YC}. In some embodiments, R^{YA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is naphthyl or an 8-10 membered bicyclic heteroaryl ring having 1-4 heteroatoms independently selected from nitrogen, oxygen, and sulfur; each of which is substituted by r⁴ instances of R^{YC}. In some embodiments, R^{YA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is phenyl or a 3-7 membered saturated or partially unsaturated monocyclic carbocyclic ring; each of which is substituted by r⁴ instances of R^{YC}. In some embodiments, R^{YA} is naphthyl or a 5-12 membered saturated or partially unsaturated bicyclic carbocyclic ring; each of which is substituted by r⁴ instances of R^{YC}. In some embodiments, R^{YA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}.

In some embodiments, R^{YA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{YC}. In some embodiments, R^{YA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{YC}. In some embodiments, R^{YA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{YC}.

In some embodiments, R^{YA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁴ instances of R^{YC}. In some embodiments, R^{YA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁴ instances of R^{YC}. In some embodiments, R^{YA} is a C₁₋₆ aliphatic chain, phenyl, or a 3-7 membered saturated or partially unsaturated monocyclic carbocyclic ring; each of which is substituted by r⁴ instances of R^{YC}.

In some embodiments, R^{YA} is selected from the groups depicted in the compounds in Table 1.

As defined generally above, R^{L} is R^{A} or R^{B} substituted by r instances of R^{LC}. In some embodiments, R^{L} is R^{A}. In some embodiments, R^{L} is R^{B} substituted by r instances of R^{LC}.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is oxo. In some embodiments, R^{L} is halogen. In some embodiments, R^{L} is —CN. In some embodiments, R^{L} is —NO₂. In some embodiments, R^{L} is —OR. In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)₂F. In some embodiments, R^{L} is —S(O)R. In some embodiments, R^{L} is —S(O)NR₂. In some embodiments, R^{L} is —S(O)(NR)R. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R. In some embodiments, R^{L} is —P(O)R₂. In some embodiments, R^{L} is —P(O)(R)OR. In some embodiments, R^{L} is —B(OR)₂. In some embodiments, R^{L} is deuterium.

In some embodiments, R^{L} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is halogen, —CN, or —NO₂. In some embodiments, R^{L} is —OR, —SR, or —NR₂. In some embodiments, R^{L} is —S(O)₂R, —S(O)₂NR₂, —S(O)₂F, —S(O)R, —S(O)NR₂, or —S(O)(NR)R. In some embodiments, R^{L} is —C(O)R, —C(O)OR, —C(O)NR₂, or —C(O)N(R)OR. In some embodiments, R^{L} is —OC(O)R or —OC(O)NR₂. In some embodiments, R^{L} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is —P(O)R₂ or —P(O)(R)OR.

In some embodiments, R^{L} is —OR, —OC(O)R, or —OC(O)NR₂. In some embodiments, R^{L} is —SR, —S(O)₂R, —S(O)₂NR₂, —S(O)₂F, —S(O)R, —S(O)NR₂, or —S(O)(NR)R. In some embodiments, R^{L} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L} is —S(O)₂R, —S(O)₂NR₂, or —S(O)₂F. In some embodiments, R^{L} is —S(O)R, —S(O)NR₂, or —S(O)(NR)R. In some embodiments, R^{L} is —SR, —S(O)₂R, or —S(O)R. In some embodiments, R^{L} is —S(O)₂NR₂, —S(O)NR₂, or —S(O)(NR)R. In some embodiments, R^{L} is —S(O)₂NR₂ or —S(O)NR₂. In some embodiments, R^{L} is —SR, —S(O)₂R, —S(O)₂NR₂, or —S(O)R.

In some embodiments, R^{L} is —N(R)C(O)OR, —N(R)C(O)R, or —N(R)C(O)NR₂. In some embodiments, R^{L} is —N(R)S(O)₂NR₂ or —N(R)S(O)₂R. In some embodiments, R^{L} is —N(R)C(O)OR or —N(R)C(O)R. In some embodiments, R^{L} is —N(R)C(O)NR₂ or —N(R)S(O)₂NR₂. In some embodiments, R^{L} is —N(R)C(O)OR, —N(R)C(O)R, or —N(R)S(O)₂R.

In some embodiments, R^{L} is —NR₂, —N(R)C(O)OR, —N(R)C(O)R, or —N(R)C(O)NR₂. In some embodiments, R^{L} is —NR₂, —N(R)C(O)OR, or —N(R)C(O)R. In some embodiments, R^{L} is —NR₂, —N(R)C(O)OR, —N(R)C(O)R, or —N(R)S(O)₂R.

In some embodiments, R^{L} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is a C₁₋₆ aliphatic chain substituted by r⁵ instances of R^{LC}In some embodiments, R^{L} is phenyl substituted by r⁵ instances of R^{LC}. In some embodiments, R^{L} is naphthyl substituted by r⁵ instances of R^{LC}. In some embodiments, R^{L} is a 5-6 membered monocyclic heteroaryl ring having 1-4 heteroatoms independently selected from nitrogen, oxygen, and sulfur; wherein said ring is substituted by r⁵ instances of R^{LC}. In some embodiments, R^{L} is an 8-10 membered bicyclic heteroaryl ring having 1-4 heteroatoms independently selected from nitrogen, oxygen, and sulfur; wherein said ring is substituted by r⁵ instances of R^{LC}. In some embodiments, R^{L} is a 3-7 membered saturated or partially unsaturated monocyclic carbocyclic ring substituted by r⁵ instances of R^{LC}. In some embodiments, R^{L} is a 5-12 membered saturated or partially unsaturated bicyclic carbocyclic ring substituted by r⁵ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; wherein said ring is substituted by r⁵ instances of R^{LC}. In some embodiments, R^{L} is a 7-12 membered saturated or partially unsaturated bicyclic heterocyclic ring having 1-4 heteroatoms independently selected from nitrogen, oxygen, and sulfur; wherein said ring is substituted by r⁵ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or naphthyl; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring or a 5-12 membered saturated or partially unsaturated bicyclic carbocyclic ring; each of which is substituted by r⁵ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or a 5-6 membered monocyclic heteroaryl ring having 1-4 heteroatoms independently selected from nitrogen, oxygen, and sulfur; each of which is substituted by r⁵ instances of R^{LC}. In some embodiments, R^{L} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is naphthyl or an 8-10 membered bicyclic heteroaryl ring having 1-4 heteroatoms independently selected from nitrogen, oxygen, and sulfur; each of which is substituted by r⁵ instances of R^{LC}. In some embodiments, R^{L} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is phenyl or a 3-7 membered saturated or partially unsaturated monocyclic carbocyclic ring; each of which is substituted by r⁵ instances of R^{LC} In some embodiments, R^{L} is naphthyl or a 5-12 membered saturated or partially unsaturated bicyclic carbocyclic ring; each of which is substituted by r⁵ instances of R^{LC}. In some embodiments, R^{L} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}.

In some embodiments, R^{L} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{LC}. In some embodiments, R^{L} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{LC}. In some embodiments, R^{L} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{LC}.

In some embodiments, R^{L} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁵ instances of R^{LC}. In some embodiments, R^{L} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁵ instances of R^{LC}. In some embodiments, R^{L} is a C₁₋₆ aliphatic chain, phenyl, or a 3-7 membered saturated or partially unsaturated monocyclic carbocyclic ring; each of which is substituted by r⁵ instances of R^{LC}.

In some embodiments, R^{L} is selected from the groups depicted in the compounds in Table 1.

As generally defined above, each instance of R^{CyAA} is independently R^{A} or R^{B} substituted by r⁶ instances of R^{CyAC}. In some embodiments, each instance of R^{CyA} is independently R^{A}. In some embodiments, each instance of R^{CyAA} is independently R^{B} substituted by r⁶ instances of R^{CyAC}.

In some embodiments, each instance of R^{CyAA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein each ring is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently a 5-6 membered monocyclic heteroaryl ring having 1-2 nitrogen atoms; wherein said ring is substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}.

In some embodiments, each instance of R^{CyAA} is independently

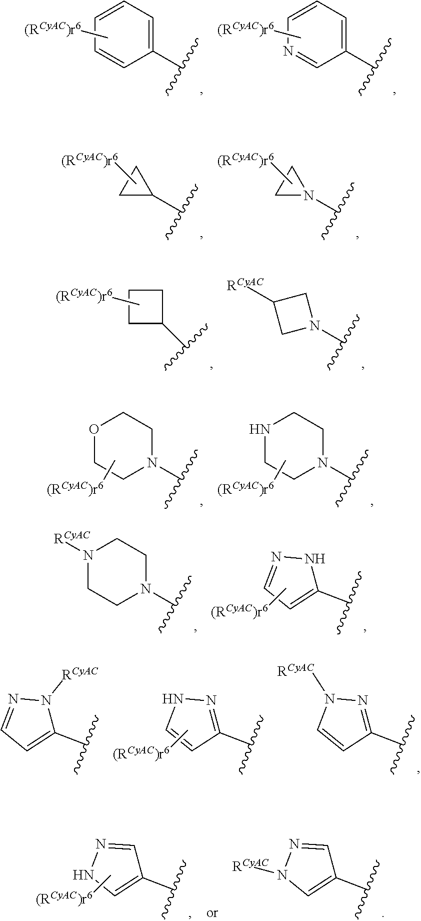

In some embodiments, each instance of R^{CyAA} is independently

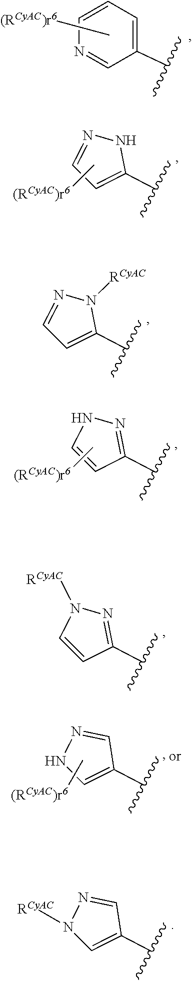

In some embodiments, each instance of R^{CyAA} is independently

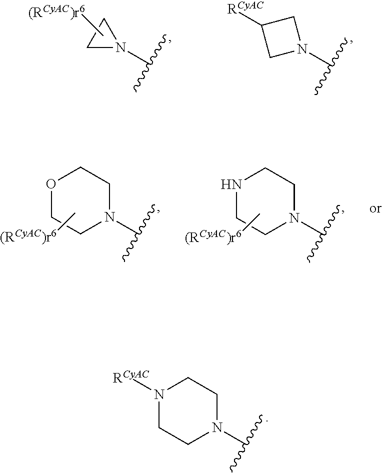

In some embodiments, each instance of R^{CyAA} is independently

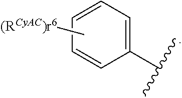

In some embodiments, each instance of R^{CyAA} is independently

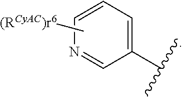

In some embodiments, each instance of R^{CyAA} is independently

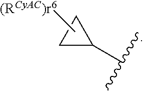

In some embodiments, each instance of R^{CyAA} is independently

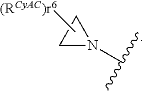

In some embodiments, each instance of R^{CyAA} is independently

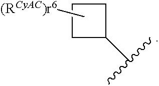

In some embodiments, each instance of R^{CyAA} is independently

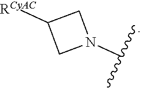

. In some embodiments, each instance of R^{CyAA} is independently

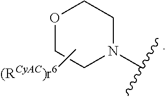

In some embodiments, each instance of R^{CyAA} is independently

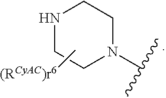

In some embodiments, each instance of R^{CyAA} is independently

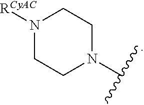

In some embodiments, each instance of R^{CyAA} is independently

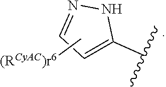

In some embodiments, each instance of R^{CyAA} is independently

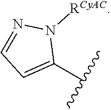

some embodiments, each instance of R^{CyAA} is independently

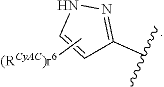

In some embodiments, each instance of R^{CyAA} is independently

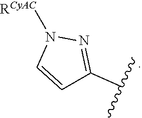

In some embodiments, each instance of R^{CyAA} is independently

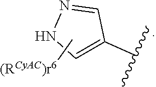

In some embodiments, each instance of R^{CyAA} is independently

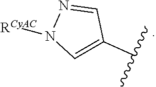

In some embodiments, each instance of R^{CyAA} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{CyAA} is independently a C₁₋₆ aliphatic that is (i) substituted with 1 or 2 groups independently selected from —O—(C₁₋₆ aliphatic), —OH, —N(C₁₋₆ aliphatic)₂, and —CN, and (ii) optionally substituted with 1, 2, or 3 atoms independently selected from halogen and deuterium. In some embodiments, each instance of R^{CyAA} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{CyAA} is independently a C₁₋₆ aliphatic substituted with 1 or 2 groups independently selected from —O—(C₁₋₆ aliphatic), —OH, —N(C₁₋₆ aliphatic)₂, and —CN.

In some embodiments, each instance of R^{CyAA} is independently a C₁₋₆ aliphatic optionally substituted with 1, 2, or 3 atoms independently selected from halogen and deuterium. In some embodiments, each instance of R^{CyAA} is independently a C₁₋₆ aliphatic substituted with 1, 2, or 3 atoms independently selected from halogen and deuterium. In some embodiments, each instance of R^{CyAA} is independently a C₁₋₆ aliphatic.

In some embodiments, each instance of R^{CyAA} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, each instance of R^{CyAA} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAA} is oxo. In some embodiments, each instance of R^{CyAA} is independently halogen. In some embodiments, each instance of R^{CyAA} is —CN. In some embodiments, each instance of R^{CyAA} is —NO₂. In some embodiments, each instance of R^{CyAA} is independently —OR. In some embodiments, each instance of R^{CyAA} is independently —SR. In some embodiments, each instance of R^{CyAA} is independently —NR₂. In some embodiments, each instance of R^{CyAA} is independently —S(O)₂R. In some embodiments, each instance of R^{CyAA} is independently —S(O)₂NR₂. In some embodiments, each instance of R^{CyAA} is —S(O)₂F. In some embodiments, each instance of R^{CyAA} is independently —S(O)R. In some embodiments, each instance of R^{CyAA} is independently —S(O)NR₂. In some embodiments, each instance of R^{CyAA} is independently —S(O)(NR)R. In some embodiments, each instance of R^{CyAA} is independently —C(O)R. In some embodiments, each instance of R^{CyAA} is independently —C(O)OR. In some embodiments, each instance of R^{CyAA} is independently —C(O)NR₂. In some embodiments, each instance of R^{CyAA} is independently —C(O)N(R)OR. In some embodiments, each instance of R^{CyAA} is independently —OC(O)R. In some embodiments, each instance of R^{CyAA} is independently —OC(O)NR₂. In some embodiments, each instance of R^{CyAA} is independently —N(R)C(O)OR. In some embodiments, each instance of R^{CyAA} is independently —N(R)C(O)R. In some embodiments, each instance of R^{CyAA} is independently —N(R)C(O)NR₂. In some embodiments, each instance of R^{CyAA} is independently —N(R)C(NR)NR₂. In some embodiments, each instance of R^{CyAA} is independently —N(R)S(O)₂NR₂. In some embodiments, each instance of R^{CyAA} is independently —N(R)S(O)₂R. In some embodiments, each instance of R^{CyAA} is independently —P(O)R₂. In some embodiments, each instance of R^{CyAA} is independently —P(O)(R)OR. In some embodiments, each instance of R^{CyAA} is independently —B(OR)₂. In some embodiments, each instance of R^{CyAA} is deuterium.

In some embodiments, each instance of R^{CyAA} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAA} is independently halogen, —CN, or —NO₂. In some embodiments, each instance of R^{CyAA} is independently —OR, —SR, or —NR₂. In some embodiments, each instance of R^{CyAA} is independently —S(O)₂R, —S(O)₂NR₂, —S(O)₂F, —S(O)R, —S(O)NR₂, or —S(O)(NR)R. In some embodiments, each instance of R^{CyAA} is independently —C(O)R, —C(O)OR, —C(O)NR₂, or —C(O)N(R)OR. In some embodiments, each instance of R^{CyAA} is independently —OC(O)R or —OC(O)NR₂. In some embodiments, each instance of R^{CyAA} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{CyAA} is independently —P(O)R₂ or —P(O)(R)OR.

In some embodiments, each instance of R^{CyAA} is independently —OR, —OC(O)R, or —OC(O)NR₂. In some embodiments, each instance of R^{CyAA} is independently —SR, —S(O)₂R, —S(O)₂NR₂, —S(O)₂F, —S(O)R, —S(O)NR₂, or —S(O)(NR)R. In some embodiments, each instance of R^{CyAA} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{CyAA} is independently —S(O)₂R, —S(O)₂NR₂, or —S(O)₂F. In some embodiments, each instance of R^{CyAA} is independently —S(O)R, —S(O)NR₂, or —S(O)(NR)R. In some embodiments, each instance of R^{CyAA} is independently —SR, —S(O)₂R, or —S(O)R. In some embodiments, each instance of R^{CyAA} is independently —S(O)₂NR₂, —S(O)NR₂, or —S(O)(NR)R. In some embodiments, each instance of R^{CyAA} is independently —S(O)₂NR₂ or —S(O)NR₂. In some embodiments, each instance of R^{CyAA} is independently —SR, —S(O)₂R, —S(O)₂NR₂, or —S(O)R.

In some embodiments, each instance of R^{CyAA} is independently —N(R)C(O)OR, —N(R)C(O)R, or —N(R)C(O)NR₂. In some embodiments, each instance of R^{CyAA} is independently —N(R)S(O)₂NR₂ or —N(R)S(O)₂R. In some embodiments, each instance of R^{CyAA} is independently —N(R)C(O)OR or —N(R)C(O)R. In some embodiments, each instance of R^{CyAA} is independently —N(R)C(O)NR₂ or —N(R)S(O)₂NR₂. In some embodiments, each instance of R^{CyAA} is independently —N(R)C(O)OR, —N(R)C(O)R, or —N(R)S(O)₂R.

In some embodiments, each instance of R^{CyAA} is independently —NR₂, —N(R)C(O)OR, —N(R)C(O)R, or —N(R)C(O)NR₂. In some embodiments, each instance of R^{CyAA} is independently —NR₂, —N(R)C(O)OR, or —N(R)C(O)R. In some embodiments, each instance of R^{CyAA} is independently —NR₂, —N(R)C(O)OR, —N(R)C(O)R, or —N(R)S(O)₂R.

In some embodiments, each instance of R^{CyAA} is independently a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain substituted by r⁶ instances of R^{CyAC}. In some embodiments, each instance of R^{Cy} is independently phenyl substituted by r⁶ instances of R^{CyAC}. In some embodiments, each instance of R^{CyAA} is independently naphthyl substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently an 8-10 membered bicyclic heteroaryl ring having 1-4 heteroatoms independently selected from nitrogen, oxygen, and sulfur; wherein said ring is substituted by r⁶ instances of R^{CyAC}. In some embodiments, each instance of R^{CyAA} is independently a 3-7 membered saturated or partially unsaturated monocyclic carbocyclic ring substituted by r⁶ instances of R^{CyAC}. In some embodiments, each instance of R^{CyAA} is independently a 5-12 membered saturated or partially unsaturated bicyclic carbocyclic ring substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}. In some embodiments, each instance of R^{CyAA} is independently a 7-12 membered saturated or partially unsaturated bicyclic heterocyclic ring having 1-4 heteroatoms independently selected from nitrogen, oxygen, and sulfur; wherein said ring is substituted by r⁶ instances of R^{CyAC}.

In some embodiments, each instance of R^{CyAA} is independently phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or naphthyl; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring or a 5-12 membered saturated or partially unsaturated bicyclic carbocyclic ring; each of which is substituted by r⁶ instances of R^{CyAC}. In some embodiments, each instance of R^{CyAA} is independently a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or a 5-6 membered monocyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently naphthyl or an 8-10 membered bicyclic heteroaryl ring having 1-4 heteroatoms independently selected from nitrogen, oxygen, and sulfur; each of which is substituted by r⁶ instances of R^{CyAC}. In some embodiments, each instance of R^{CyAA} is independently a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently phenyl or a 3-7 membered saturated or partially unsaturated monocyclic carbocyclic ring; each of which is substituted by r⁶ instances of R^{CyAC}. In some embodiments, each instance of R^{CyAA} is independently naphthyl or a 5-12 membered saturated or partially unsaturated bicyclic carbocyclic ring; each of which is substituted by r⁶ instances of R^{CyAC}. In some embodiments, each instance of R^{CyAA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{CyAC}. In some embodiments, each instance of R^{CyAA} is independently a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{CyAC}. In some embodiments, each instance of R^{CyAA} is independently a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}.

In some embodiments, each instance of R^{CyAA} is independently a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁶ instances of R^{CyAC}. In some embodiments, each instance of R^{CyAA} is independently a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁶ instances of R^{CyAC}. In some embodiments, each instance of R^{CyAA} is independently a C₁₋₆ aliphatic chain, phenyl, or a 3-7 membered saturated or partially unsaturated monocyclic carbocyclic ring; each of which is substituted by r⁶ instances of R^{CyAC}.

In some embodiments, each instance of R^{CyAA} is independently selected from the groups depicted in the compounds in Table 1.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{A} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, R^{A} is halogen. In some embodiments, R^{A} is —CN. In some embodiments, R^{A} is —NO₂. In some embodiments, R^{A} is —OR. In some embodiments, R^{A} is —SF₅. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)₂F. In some embodiments, R^{A} is —S(O)R. In some embodiments, R^{A} is —S(O)NR₂. In some embodiments, R^{A} is —S(O)(NR)R. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR. In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, R^{A} is —P(O)R₂. In some embodiments, R^{A} is —P(O)(R)OR. In some embodiments, R^{A} is —B(OR)₂. In some embodiments, R^{A} is deuterium.

In some embodiments, R^{A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is halogen, —CN, or —NO₂. In some embodiments, R^{A} is —OR, —SR, or —NR₂. In some embodiments, R^{A} is —S(O)₂R, —S(O)₂NR₂, —S(O)₂F, —S(O)R, —S(O)NR₂, or —S(O)(NR)R. In some embodiments, R^{A} is —C(O)R, —C(O)OR, —C(O)NR₂, or —C(O)N(R)OR. In some embodiments, R^{A} is —OC(O)R or —OC(O)NR₂. In some embodiments, R^{A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{A} is —P(O)R₂ or —P(O)(R)OR.

In some embodiments, R^{A} is —OR, —OC(O)R, or —OC(O)NR₂. In some embodiments, R^{A} is —SR, —S(O)₂R, —S(O)₂NR₂, —S(O)₂F, —S(O)R, —S(O)NR₂, or —S(O)(NR)R. In some embodiments, R^{A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{A} is —S(O)₂R, —S(O)₂NR₂, or —S(O)₂F. In some embodiments, R^{A} is —S(O)R, —S(O)NR₂, or —S(O)(NR)R. In some embodiments, R^{A} is —SR, —S(O)₂R, or —S(O)R. In some embodiments, R^{A} is —S(O)₂NR₂, —S(O)NR₂, or —S(O)(NR)R. In some embodiments, R^{A} is —S(O)₂NR₂ or —S(O)NR₂. In some embodiments, R^{A} is —SR, —S(O)₂R, —S(O)₂NR₂, or —S(O)R.

In some embodiments, R^{A} is —N(R)C(O)OR, —N(R)C(O)R, or —N(R)C(O)NR₂. In some embodiments, R^{A} is —N(R)S(O)₂NR₂ or —N(R)S(O)₂R. In some embodiments, R^{A} is —N(R)C(O)OR or —N(R)C(O)R. In some embodiments, R^{A} is —N(R)C(O)NR₂ or —N(R)S(O)₂NR₂. In some embodiments, R^{A} is —N(R)C(O)OR, —N(R)C(O)R, or —N(R)S(O)₂R.

In some embodiments, R^{A} is —NR₂, —N(R)C(O)OR, —N(R)C(O)R, or —N(R)C(O)NR₂. In some embodiments, R^{A} is —NR₂, —N(R)C(O)OR, or —N(R)C(O)R. In some embodiments, R^{A} is —NR₂, —N(R)C(O)OR, —N(R)C(O)R, or —N(R)S(O)₂R.

In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain. In some embodiments, R^{B} is phenyl. In some embodiments, R^{B} is naphthyl. In some embodiments, R^{B} is cubanyl. In some embodiments, R^{B} is adamantyl. In some embodiments, R^{B} is a 5-6 membered monocyclic heteroaryl ring having 1-4 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring. In some embodiments, R^{B} is a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur. In some embodiments, R^{B} is a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{B} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{B} is phenyl or naphthyl. In some embodiments, R^{B} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl or a 5-6 membered monocyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{B} is naphthyl or an 8-10 membered bicyclic heteroaryl ring having 1-4 heteroatoms independently selected from nitrogen, oxygen, and sulfur. In some embodiments, R^{B} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{B} is phenyl or a 3-7 membered saturated or partially unsaturated monocyclic carbocyclic ring. In some embodiments, R^{B} is naphthyl or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{B} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{B} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{B} is a C₁₋₆ aliphatic chain, phenyl, or a 3-7 membered saturated or partially unsaturated monocyclic carbocyclic ring.

In some embodiments, R^{B} is selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{1C} is oxo. In some embodiments, R^{1C} is deuterium. In some embodiments, each instance of R^{1C} is independently halogen. In some embodiments, R^{1C} is —CN. In some embodiments, R^{1C} is —NO₂. In some embodiments, R^{1C} is —OR. In some embodiments, R^{1C} is —SR. In some embodiments, R^{1C} is —NR₂. In some embodiments, R^{1C} is —S(O)₂R. In some embodiments, R^{1C} is —S(O)₂NR₂. In some embodiments, R^{1C} is —S(O)₂F. In some embodiments, R^{1C} is —S(O)R. In some embodiments, R^{1C} is —S(O)NR₂. In some embodiments, R^{1C} is —S(O)(NR)R. In some embodiments, R^{1C} is —C(O)R. In some embodiments, R^{1C} is —C(O)OR. In some embodiments, R^{1C} is —C(O)NR₂. In some embodiments, R^{1C} is —C(O)N(R)OR. In some embodiments, R^{1C} is —OC(O)R. In some embodiments, R^{1C} is —OC(O)NR₂. In some embodiments, R^{1C} is —N(R)C(O)OR. In some embodiments, R^{1C} is —N(R)C(O)R. In some embodiments, R^{1C} is —N(R)C(O)NR₂. In some embodiments, R^{1C} is —N(R)C(NR)NR₂. In some embodiments, R^{1C} is —N(R)S(O)₂NR₂. In some embodiments, R^{1C} is —N(R)S(O)₂R. In some embodiments, R^{1C} is —P(O)R₂. In some embodiments, R^{1C} is —P(O)(R)OR. In some embodiments, R^{1C} is —B(OR)₂.

In some embodiments, each instance of R^{1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{1C} is independently halogen, —CN, or —NO₂. In some embodiments, each instance of R^{1C} is independently —OR, —SR, or —NR₂. In some embodiments, each instance of R^{1C} is independently —S(O)₂R, —S(O)₂NR₂, —S(O)₂F, —S(O)R, —S(O)NR₂, or —S(O)(NR)R. In some embodiments, each instance of R^{1C} is independently —C(O)R, —C(O)OR, —C(O)NR₂, or —C(O)N(R)OR. In some embodiments, each instance of R^{1C} is independently —OC(O)R or —OC(O)NR₂. In some embodiments, each instance of R^{1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{1C} is independently —P(O)R₂ or —P(O)(R)OR.

In some embodiments, each instance of R^{1C} is independently —OR, —OC(O)R, or —OC(O)NR₂. In some embodiments, each instance of R^{1C} is independently —SR, —S(O)₂R, —S(O)₂NR₂, —S(O)₂F, —S(O)R, —S(O)NR₂, or —S(O)(NR)R. In some embodiments, each instance of R^{1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{1C} is independently —S(O)₂R, —S(O)₂NR₂, or —S(O)₂F. In some embodiments, each instance of R^{1C} is independently —S(O)R, —S(O)NR₂, or —S(O)(NR)R. In some embodiments, each instance of R^{1C} is independently —SR, —S(O)₂R, or —S(O)R. In some embodiments, each instance of R^{1C} is independently —S(O)₂NR₂, —S(O)NR₂, or —S(O)(NR)R. In some embodiments, each instance of R^{1C} is independently —S(O)₂NR₂ or —S(O)NR₂. In some embodiments, each instance of R^{1C} is independently —SR, —S(O)₂R, —S(O)₂NR₂, or —S(O)R.

In some embodiments, each instance of R^{1C} is independently —N(R)C(O)OR, —N(R)C(O)R, or —N(R)C(O)NR₂. In some embodiments, each instance of R^{1C} is independently —N(R)S(O)₂NR₂ or —N(R)S(O)₂R. In some embodiments, each instance of R^{1C} is independently —N(R)C(O)OR or —N(R)C(O)R. In some embodiments, each instance of R^{1C} is independently —N(R)C(O)NR₂ or —N(R)S(O)₂NR₂. In some embodiments, each instance of R^{1C} is independently —N(R)C(O)OR, —N(R)C(O)R, or —N(R)S(O)₂R.

In some embodiments, each instance of R^{1C} is independently —NR₂, —N(R)C(O)OR, —N(R)C(O)R, or —N(R)C(O)NR₂. In some embodiments, each instance of R^{1C} is independently —NR₂, —N(R)C(O)OR, or —N(R)C(O)R. In some embodiments, each instance of R^{1C} is independently —NR₂, —N(R)C(O)OR, —N(R)C(O)R, or —N(R)S(O)₂R.

In some embodiments, each instance of R^{1C} is independently an optionally substituted C₁₋₆ aliphatic. In some embodiments, each instance of R^{1C} is independently an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic. In some embodiments, R^{1C} is phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur. In some embodiments, each instance of R^{1C} is independently a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{1C} is independently phenyl or a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and

In some embodiments, each instance of R^{1C} is independently halogen, —CN, —O-(optionally substituted C₁₋₆ aliphatic), or an optionally substituted C₁₋₆ aliphatic. In some embodiments, each instance of R^{1C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{1C} is independently halogen or C₁₋₃ aliphatic optionally substituted with 1-3 halogen. In some embodiments, each instance of R^{1C} is independently fluorine, chlorine, —CH₃, —CHF₂, or —CF₃.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{1C} is independently selected from the groups depicted in the compounds in Table 1.

As defined generally above, each instance of R^{2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{2C} is oxo. In some embodiments, R^{2C} is deuterium. In some embodiments, each instance of R^{2C} is independently halogen. In some embodiments, R^{2C} is —CN. In some embodiments, R^{2C} is —NO₂. In some embodiments, R^{2C} is —OR. In some embodiments, R^{2C} is —SR. In some embodiments, R^{2C} is —NR₂. In some embodiments, R^{2C} is —S(O)₂R. In some embodiments, R^{2C} is —S(O)₂NR₂. In some embodiments, R^{2C} is —S(O)₂F. In some embodiments, R^{2C} is —S(O)R. In some embodiments, R^{2C} is —S(O)NR₂. In some embodiments, R^{2C} is —S(O)(NR)R. In some embodiments, R^{2C} is —C(O)R. In some embodiments, R^{2C} is —C(O)OR. In some embodiments, R^{2C} is —C(O)NR₂. In some embodiments, R^{2C} is —C(O)N(R)OR. In some embodiments, R^{2C} is —OC(O)R. In some embodiments, R^{2C} is —OC(O)NR₂. In some embodiments, R^{2C} is —N(R)C(O)OR. In some embodiments, R^{2C} is —N(R)C(O)R. In some embodiments, R^{2C} is —N(R)C(O)NR₂. In some embodiments, R^{2C} is —N(R)C(NR)NR₂. In some embodiments, R^{2C} is —N(R)S(O)₂NR₂. In some embodiments, R^{2C} is —N(R)S(O)₂R. In some embodiments, R^{2C} is —P(O)R₂. In some embodiments, R^{2C} is —P(O)(R)OR. In some embodiments, R^{2C} is —B(OR)₂.

In some embodiments, each instance of R^{2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{2C} is independently halogen, —CN, or —NO₂. In some embodiments, each instance of R^{2C} is independently —OR, —SR, or —NR₂. In some embodiments, each instance of R^{2C} is independently —S(O)₂R, —S(O)₂NR₂, —S(O)₂F, —S(O)R, —S(O)NR₂, or —S(O)(NR)R. In some embodiments, each instance of R^{2C} is independently —C(O)R, —C(O)OR, —C(O)NR₂, or —C(O)N(R)OR. In some embodiments, each instance of R^{2C} is independently —OC(O)R or —OC(O)NR₂. In some embodiments, each instance of R^{2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{2C} is independently —P(O)R₂ or —P(O)(R)OR.

In some embodiments, each instance of R^{2C} is independently —OR, —OC(O)R, or —OC(O)NR₂. In some embodiments, each instance of R^{2C} is independently —SR, —S(O)₂R, —S(O)₂NR₂, —S(O)₂F, —S(O)R, —S(O)NR₂, or —S(O)(NR)R. In some embodiments, each instance of R^{2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{2C} is independently —S(O)₂R, —S(O)₂NR₂, or —S(O)₂F. In some embodiments, each instance of R^{2C} is independently —S(O)R, —S(O)NR₂, or —S(O)(NR)R. In some embodiments, each instance of R^{2C} is independently —SR, —S(O)₂R, or —S(O)R. In some embodiments, each instance of R^{2C} is independently —S(O)₂NR₂, —S(O)NR₂, or —S(O)(NR)R. In some embodiments, each instance of R^{2C} is independently —S(O)₂NR₂ or —S(O)NR₂. In some embodiments, each instance of R^{2C} is independently —SR, —S(O)₂R, —S(O)₂NR₂, or —S(O)R.

In some embodiments, each instance of R^{2C} is independently —N(R)C(O)OR, —N(R)C(O)R, or —N(R)C(O)NR₂. In some embodiments, each instance of R^{2C} is independently —N(R)S(O)₂NR₂ or —N(R)S(O)₂R. In some embodiments, each instance of R^{2C} is independently —N(R)C(O)OR or —N(R)C(O)R. In some embodiments, each instance of R^{2C} is independently —N(R)C(O)NR₂ or —N(R)S(O)₂NR₂. In some embodiments, each instance of R^{2C} is independently —N(R)C(O)OR, —N(R)C(O)R, or —N(R)S(O)₂R.

In some embodiments, each instance of R^{2C} is independently —NR₂, —N(R)C(O)OR, —N(R)C(O)R, or —N(R)C(O)NR₂. In some embodiments, each instance of R^{2C} is independently —NR₂, —N(R)C(O)OR, or —N(R)C(O)R. In some embodiments, each instance of R^{2C} is independently —NR₂, —N(R)C(O)OR, —N(R)C(O)R, or —N(R)S(O)₂R.

In some embodiments, each instance of R^{2C} is independently an optionally substituted C₁₋₆ aliphatic. In some embodiments, each instance of R^{2C} is independently an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic. In some embodiments, R^{2C} is phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur. In some embodiments, each instance of R^{2C} is independently a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{2C} is independently phenyl or a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently halogen, —CN, —O-(optionally substituted C₁₋₆ aliphatic), or an optionally substituted C₁₋₆ aliphatic. In some embodiments, each instance of R^{2C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{2C} is independently halogen or C₁₋₃ aliphatic optionally substituted with 1-3 halogen. In some embodiments, each instance of R^{2C} is independently fluorine, chlorine, —CH₃, —CHF₂, or —CF₃.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{2C} is independently selected from the groups depicted in the compounds in Table 1.

As defined generally above, each instance of R^{XC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{XC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{XC} is oxo. In some embodiments, R^{XC} is deuterium. In some embodiments, each instance of R^{XC} is independently halogen. In some embodiments, R^{XC} is —CN. In some embodiments, R^{XC} is —NO₂. In some embodiments, R^{XC} is —OR. In some embodiments, R^{XC} is —SR. In some embodiments, R^{XC} is —NR₂. In some embodiments, R^{XC} is —S(O)₂R. In some embodiments, R^{XC} is —S(O)₂NR₂. In some embodiments, R^{XC} is —S(O)₂F. In some embodiments, R^{XC} is —S(O)R. In some embodiments, R^{XC} is —S(O)NR₂. In some embodiments, R^{XC} is —S(O)(NR)R. In some embodiments, R^{XC} is —C(O)R. In some embodiments, R^{XC} is —C(O)OR. In some embodiments, R^{XC} is —C(O)NR₂. In some embodiments, R^{XC} is —C(O)N(R)OR. In some embodiments, R^{XC} is —OC(O)R. In some embodiments, R^{XC} is —OC(O)NR₂. In some embodiments, R^{XC} is —N(R)C(O)OR. In some embodiments, R^{XC} is —N(R)C(O)R. In some embodiments, R^{XC} is —N(R)C(O)NR₂. In some embodiments, R^{XC} is —N(R)C(NR)NR₂. In some embodiments, R^{XC} is —N(R)S(O)₂NR₂. In some embodiments, R^{XC} is —N(R)S(O)₂R. In some embodiments, R^{XC} is —P(O)R₂. In some embodiments, R^{XC} is —P(O)(R)OR. In some embodiments, R^{XC} is —B(OR)₂.

In some embodiments, each instance of R^{XC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{XC} is independently halogen, —CN, or —NO₂. In some embodiments, each instance of R^{XC} is independently —OR, —SR, or —NR₂. In some embodiments, each instance of R^{XC} is independently —S(O)₂R, —S(O)₂NR₂, —S(O)₂F, —S(O)R, —S(O)NR₂, or —S(O)(NR)R. In some embodiments, each instance of R^{XC} is independently —C(O)R, —C(O)OR, —C(O)NR₂, or —C(O)N(R)OR. In some embodiments, each instance of R^{XC} is independently —OC(O)R or —OC(O)NR₂. In some embodiments, each instance of R^{XC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{XC} is independently —P(O)R₂ or —P(O)(R)OR.

In some embodiments, each instance of R^{XC} is independently —OR, —OC(O)R, or —OC(O)NR₂. In some embodiments, each instance of R^{XC} is independently —SR, —S(O)₂R, —S(O)₂NR₂, —S(O)₂F, —S(O)R, —S(O)NR₂, or —S(O)(NR)R. In some embodiments, each instance of R^{XC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{XC} is independently —S(O)₂R, —S(O)₂NR₂, or —S(O)₂F. In some embodiments, each instance of R^{XC} is independently —S(O)R, —S(O)NR₂, or —S(O)(NR)R. In some embodiments, each instance of R^{XC} is independently —SR, —S(O)₂R, or —S(O)R. In some embodiments, each instance of R^{XC} is independently —S(O)₂NR₂, —S(O)NR₂, or —S(O)(NR)R. In some embodiments, each instance of R^{XC} is independently —S(O)₂NR₂ or —S(O)NR₂. In some embodiments, each instance of R^{XC} is independently —SR, —S(O)₂R, —S(O)₂NR₂, or —S(O)R.

In some embodiments, each instance of R^{XC} is independently —N(R)C(O)OR, —N(R)C(O)R, or —N(R)C(O)NR₂. In some embodiments, each instance of R^{XC} is independently —N(R)S(O)₂NR₂ or —N(R)S(O)₂R. In some embodiments, each instance of R^{XC} is independently —N(R)C(O)OR or —N(R)C(O)R. In some embodiments, each instance of R^{XC} is independently —N(R)C(O)NR₂ or —N(R)S(O)₂NR₂. In some embodiments, each instance of R^{XC} is independently —N(R)C(O)OR, —N(R)C(O)R, or —N(R)S(O)₂R.

In some embodiments, each instance of R^{XC} is independently —NR₂, —N(R)C(O)OR, —N(R)C(O)R, or —N(R)C(O)NR₂. In some embodiments, each instance of R^{XC} is independently —NR₂, —N(R)C(O)OR, or —N(R)C(O)R. In some embodiments, each instance of R^{XC} is independently —NR₂, —N(R)C(O)OR, —N(R)C(O)R, or —N(R)S(O)₂R.

In some embodiments, each instance of R^{XC} is independently an optionally substituted C₁₋₆ aliphatic. In some embodiments, each instance of R^{XC} is independently an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and

In some embodiments, each instance of R^{XC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic. In some embodiments, R^{XC} is phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur. In some embodiments, each instance of R^{XC} is independently a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{XC} is independently phenyl or a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently selected from the groups depicted in the compounds in Table 1.

As defined generally above, each instance of R^{YC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{YC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{YC} is oxo. In some embodiments, R^{YC} is deuterium. In some embodiments, each instance of R^{YC} is independently halogen. In some embodiments, R^{YC} is —CN. In some embodiments, R^{YC} is —NO₂. In some embodiments, R^{YC} is —OR. In some embodiments, R^{YC} is —SR. In some embodiments, R^{YC} is —NR₂. In some embodiments, R^{YC} is —S(O)₂R. In some embodiments, R^{YC} is —S(O)₂NR₂. In some embodiments, R^{YC}is —S(O)₂F. In some embodiments, R^{YC} is —S(O)R. In some embodiments, R^{YC} is —S(O)NR₂. In some embodiments, R^{YC} is —S(O)(NR)R. In some embodiments, R^{YC} is —C(O)R. In some embodiments, R^{YC} is —C(O)OR. In some embodiments, R^{YC} is —C(O)NR₂. In some embodiments, R^{YC} is —C(O)N(R)OR. In some embodiments, R^{YC} is —OC(O)R. In some embodiments, R^{YC} is —OC(O)NR₂. In some embodiments, R^{YC} is —N(R)C(O)OR. In some embodiments, R^{YC} is —N(R)C(O)R. In some embodiments, R^{YC} is —N(R)C(O)NR₂. In some embodiments, R^{YC} is —N(R)C(NR)NR₂. In some embodiments, R^{YC} is —N(R)S(O)₂NR₂. In some embodiments, R^{YC} is —N(R)S(O)₂R. In some embodiments, R^{YC} is —P(O)R₂. In some embodiments, R^{YC} is —P(O)(R)OR. In some embodiments, R^{YC} is —B(OR)₂.

In some embodiments, each instance of R^{YC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{YC} is independently halogen, —CN, or —NO₂. In some embodiments, each instance of R^{YC} is independently —OR, —SR, or —NR₂. In some embodiments, each instance of R^{YC} is independently —S(O)₂R, —S(O)₂NR₂, —S(O)₂F, —S(O)R, —S(O)NR₂, or —S(O)(NR)R. In some embodiments, each instance of R^{YC} is independently —C(O)R, —C(O)OR, —C(O)NR₂, or —C(O)N(R)OR. In some embodiments, each instance of R^{YC} is independently —OC(O)R or —OC(O)NR₂. In some embodiments, each instance of R^{YC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{YC} is independently —P(O)R₂ or —P(O)(R)OR.

In some embodiments, each instance of R^{YC} is independently —OR, —OC(O)R, or —OC(O)NR₂. In some embodiments, each instance of R^{YC} is independently —SR, —S(O)₂R, —S(O)₂NR₂, —S(O)₂F, —S(O)R, —S(O)NR₂, or —S(O)(NR)R. In some embodiments, each instance of R^{YC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{YC} is independently —S(O)₂R, —S(O)₂NR₂, or —S(O)₂F. In some embodiments, each instance of R^{YC} is independently —S(O)R, —S(O)NR₂, or —S(O)(NR)R. In some embodiments, each instance of R^{YC} is independently —SR, —S(O)₂R, or —S(O)R. In some embodiments, each instance of R^{YC} is independently —S(O)₂NR₂, —S(O)NR₂, or —S(O)(NR)R. In some embodiments, each instance of R^{YC} is independently —S(O)₂NR₂ or —S(O)NR₂. In some embodiments, each instance of R^{YC} is independently —SR, —S(O)₂R, —S(O)₂NR₂, or —S(O)R.

In some embodiments, each instance of R^{YC} is independently —N(R)C(O)OR, —N(R)C(O)R, or —N(R)C(O)NR₂. In some embodiments, each instance of R^{YC} is independently —N(R)S(O)₂NR₂ or —N(R)S(O)₂R. In some embodiments, each instance of R^{YC} is independently —N(R)C(O)OR or —N(R)C(O)R. In some embodiments, each instance of R^{YC} is independently —N(R)C(O)NR₂ or —N(R)S(O)₂NR₂. In some embodiments, each instance of R^{YC} is independently —N(R)C(O)OR, —N(R)C(O)R, or —N(R)S(O)₂R.

In some embodiments, each instance of R^{YC} is independently —NR₂, —N(R)C(O)OR, —N(R)C(O)R, or —N(R)C(O)NR₂. In some embodiments, each instance of R^{YC} is independently —NR₂, —N(R)C(O)OR, or —N(R)C(O)R. In some embodiments, each instance of R^{YC} is independently —NR₂, —N(R)C(O)OR, —N(R)C(O)R, or —N(R)S(O)₂R.

In some embodiments, each instance of R^{YC} is independently an optionally substituted C₁₋₆ aliphatic. In some embodiments, each instance of R^{YC} is independently an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic. In some embodiments, R^{YC} is phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur. In some embodiments, each instance of R^{YC} is independently a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{YC} is independently phenyl or a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{YC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{YC} is independently halogen, —CN, —OH, —O-(optionally substituted C₁₋₃ aliphatic), or an optionally substituted C₁₋₃ aliphatic. In some embodiments, each instance of R^{YC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently fluorine, chlorine, —OH, —OCH₃, —OCF₃, —CH₃, —CHF₂, or —CF₃. In some embodiments, each instance of R^{YC} is independently fluorine or —OH.

In some embodiments, each instance of R^{YC} is independently oxo, deuterium, halogen, —CN, —OH, —O-(optionally substituted C₁₋₃ aliphatic), or an optionally substituted C₁₋₃ aliphatic. In some embodiments, each instance of R^{YC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{YC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently oxo, deuterium, fluorine, chlorine, —CN, —OH, —OCH₃, —OCF₃, —CH₃, —CHF₂, or —CF₃. In some embodiments, each instance of R^{YC} is independently oxo, deuterium, —CN, fluorine, or —OH. In some embodiments, each instance of R^{YC} is independently oxo, deuterium, —CN, —CH₃, or —CHF₂. In some embodiments, each instance of R^{YC} is independently deuterium, —CN, —CH₃, or —CHF₂.

In some embodiments, each instance of R^{YC} is independently oxo, halogen, —CN, —OH, —O-(optionally substituted C₁₋₃ aliphatic), or an optionally substituted C₁₋₃ aliphatic. In some embodiments, each instance of R^{YC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{YC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently oxo, fluorine, chlorine, —CN, —OH, —OCH₃, —OCF₃, —CH₃, —CHF₂, or —CF₃. In some embodiments, each instance of R^{YC} is independently oxo, —CN, fluorine, or —OH. In some embodiments, each instance of R^{YC} is independently oxo, —CN, —CH₃, or —CHF₂. In some embodiments, each instance of R^{YC} is independently —CN, —CH₃, or —CHF₂.

In some embodiments, each instance of R^{YC} is independently selected from the groups depicted in the compounds in Table 1.

As defined generally above, each instance of R^{LC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{LC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{LC} is oxo. In some embodiments, R^{LC} is deuterium. In some embodiments, each instance of R^{LC} is independently halogen. In some embodiments, R^{LC} is —CN. In some embodiments, R^{LC} is —NO₂. In some embodiments, R^{LC} is —OR. In some embodiments, R^{LC} is —SR. In some embodiments, R^{LC} is —NR₂. In some embodiments, R^{LC} is —S(O)₂R. In some embodiments, R^{LC} is —S(O)₂NR₂. In some embodiments, R^{LC} is —S(O)₂F. In some embodiments, R^{LC} is —S(O)R. In some embodiments, R^{LC} is —S(O)NR₂. In some embodiments, R^{LC} is —S(O)(NR)R. In some embodiments, R^{LC} is —C(O)R. In some embodiments, R^{LC} is —C(O)OR. In some embodiments, R^{LC} is —C(O)NR₂. In some embodiments, R^{LC} is —C(O)N(R)OR. In some embodiments, R^{LC} is —OC(O)R. In some embodiments, R^{LC} is —OC(O)NR₂. In some embodiments, R^{LC} is —N(R)C(O)OR. In some embodiments, R^{LC} is —N(R)C(O)R. In some embodiments, R^{LC} is —N(R)C(O)NR₂. In some embodiments, R^{LC} is —N(R)C(NR)NR₂. In some embodiments, R^{LC} is —N(R)S(O)₂NR₂. In some embodiments, R^{LC} is —N(R)S(O)₂R. In some embodiments, R^{LC} is —P(O)R₂. In some embodiments, R^{LC} is —P(O)(R)OR. In some embodiments, R^{LC} is —B(OR)₂.

In some embodiments, each instance of R^{LC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{LC} is independently halogen, —CN, or —NO₂. In some embodiments, each instance of R^{LC} is independently —OR, —SR, or —NR₂. In some embodiments, each instance of R^{LC} is independently —S(O)₂R, —S(O)₂NR₂, —S(O)₂F, —S(O)R, —S(O)NR₂, or —S(O)(NR)R. In some embodiments, each instance of R^{LC} is independently —C(O)R, —C(O)OR, —C(O)NR₂, or —C(O)N(R)OR. In some embodiments, each instance of R^{LC} is independently —OC(O)R or —OC(O)NR₂. In some embodiments, each instance of R^{LC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{LC} is independently —P(O)R₂ or —P(O)(R)OR.

In some embodiments, each instance of R^{LC} is independently —OR, —OC(O)R, or —OC(O)NR₂. In some embodiments, each instance of R^{LC} is independently —SR, —S(O)₂R, —S(O)₂NR₂, —S(O)₂F, —S(O)R, —S(O)NR₂, or —S(O)(NR)R. In some embodiments, each instance of R^{LC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{LC} is independently —S(O)₂R, —S(O)₂NR₂, or —S(O)₂F. In some embodiments, each instance of R^{LC} is independently —S(O)R, —S(O)NR₂, or —S(O)(NR)R. In some embodiments, each instance of R^{LC} is independently —SR, —S(O)₂R, or —S(O)R. In some embodiments, each instance of R^{LC} is independently —S(O)₂NR₂, —S(O)NR₂, or —S(O)(NR)R. In some embodiments, each instance of R^{LC} is independently —S(O)₂NR₂ or —S(O)NR₂. In some embodiments, each instance of R^{LC} is independently —SR, —S(O)₂R, —S(O)₂NR₂, or —S(O)R.

In some embodiments, each instance of R^{LC} is independently —N(R)C(O)OR, —N(R)C(O)R, or —N(R)C(O)NR₂. In some embodiments, each instance of R^{LC} is independently —N(R)S(O)₂NR₂ or —N(R)S(O)₂R. In some embodiments, each instance of R^{LC} is independently —N(R)C(O)OR or —N(R)C(O)R. In some embodiments, each instance of R^{LC} is independently —N(R)C(O)NR₂ or —N(R)S(O)₂NR₂. In some embodiments, each instance of R^{LC} is independently —N(R)C(O)OR, —N(R)C(O)R, or —N(R)S(O)₂R.

In some embodiments, each instance of R^{LC} is independently —NR₂, —N(R)C(O)OR, —N(R)C(O)R, or —N(R)C(O)NR₂. In some embodiments, each instance of R^{LC} is independently —NR₂, —N(R)C(O)OR, or —N(R)C(O)R. In some embodiments, each instance of R^{LC} is independently —NR₂, —N(R)C(O)OR, —N(R)C(O)R, or —N(R)S(O)₂R.

In some embodiments, each instance of R^{LC} is independently an optionally substituted C₁₋₆ aliphatic. In some embodiments, each instance of R^{LC} is independently an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic. In some embodiments, R^{LC} is phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur. In some embodiments, each instance of R^{LC} is independently a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{LC} is independently phenyl or a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently selected from the groups depicted in the compounds in Table 1.

As defined generally above, each instance of R^{CyAC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{CyAC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{CyAC} is oxo. In some embodiments, R^{CyAC} is deuterium. In some embodiments, each instance of R^{CyAC} is independently halogen. In some embodiments, R^{CyAC} is —CN. In some embodiments, R^{CyAC} is —NO₂. In some embodiments, R^{CyAC} is —OR. In some embodiments, R^{CyAC} is —SR. In some embodiments, R^{CyAC} is —NR₂. In some embodiments, R^{CyAC} is —S(O)₂R. In some embodiments, R^{CyAC} is —S(O)₂NR₂. In some embodiments, R^{CyAC} is —S(O)₂F. In some embodiments, R^{CyAC} is —S(O)R. In some embodiments, R^{CyAC} is —S(O)NR₂. In some embodiments, R^{CyAC} is —S(O)(NR)R. In some embodiments, R^{CyAC} is —C(O)R. In some embodiments, R^{CyAC} is —C(O)OR. In some embodiments, R^{CyAC} is —C(O)NR₂. In some embodiments, R^{CyAC} is —C(O)N(R)OR. In some embodiments, R^{CyAC} is —OC(O)R. In some embodiments, R^{CyAC} is —OC(O)NR₂. In some embodiments, R^{CyAC} is —N(R)C(O)OR. In some embodiments, R^{CyAC} is —N(R)C(O)R. In some embodiments, R^{CyAC} is —N(R)C(O)NR₂. In some embodiments, R^{CyAC} is —N(R)C(NR)NR₂. In some embodiments, R^{CyAC} is —N(R)S(O)₂NR₂. In some embodiments, R^{CyAC} is —N(R)S(O)₂R. In some embodiments, R^{CyAC} is —P(O)R₂. In some embodiments, R^{CyAC} is —P(O)(R)OR. In some embodiments, R^{CyAC} is —B(OR)₂.

In some embodiments, each instance of R^{CyAC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{CyAC} is independently halogen, —CN, or —NO₂. In some embodiments, each instance of R^{CyAC} is independently —OR, —SR, or —NR₂. In some embodiments, each instance of R^{CyAC} is independently —S(O)₂R, —S(O)₂NR₂, —S(O)₂F, —S(O)R, —S(O)NR₂, or —S(O)(NR)R. In some embodiments, each instance of R^{CyAC} is independently —C(O)R, —C(O)OR, —C(O)NR₂, or —C(O)N(R)OR. In some embodiments, each instance of R^{CyAC} is independently —OC(O)R or —OC(O)NR₂. In some embodiments, each instance of R^{CyAC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{CyAC} is independently —P(O)R₂ or —P(O)(R)OR.

In some embodiments, each instance of R^{CyAC} is independently —OR, —OC(O)R, or —OC(O)NR₂. In some embodiments, each instance of R^{CyAC} is independently —SR, —S(O)₂R, —S(O)₂NR₂, —S(O)₂F, —S(O)R, —S(O)NR₂, or —S(O)(NR)R. In some embodiments, each instance of R^{CyAC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{CyAC} is independently —S(O)₂R, —S(O)₂NR₂, or —S(O)₂F. In some embodiments, each instance of R^{CyAC} is independently —S(O)R, —S(O)NR₂, or —S(O)(NR)R. In some embodiments, each instance of R^{CyAC} is independently —SR, —S(O)₂R, or —S(O)R. In some embodiments, each instance of R^{CyAC} is independently —S(O)₂NR₂, —S(O)NR₂, or —S(O)(NR)R. In some embodiments, each instance of R^{CyAC} is independently —S(O)₂NR₂ or —S(O)NR₂. In some embodiments, each instance of R^{CyAC} is independently —SR, —S(O)₂R, —S(O)₂NR₂, or —S(O)R.

In some embodiments, each instance of R^{CyAC} is independently —N(R)C(O)OR, —N(R)C(O)R, or —N(R)C(O)NR₂. In some embodiments, each instance of R^{CyAC} is independently —N(R)S(O)₂NR₂ or —N(R)S(O)₂R. In some embodiments, each instance of R^{CyAC} is independently —N(R)C(O)OR or —N(R)C(O)R. In some embodiments, each instance of R^{CyAC} is independently —N(R)C(O)NR₂ or —N(R)S(O)₂NR₂. In some embodiments, each instance of R^{CyAC} is independently —N(R)C(O)OR, —N(R)C(O)R, or —N(R)S(O)₂R.

In some embodiments, each instance of R^{CyAC} is independently —NR₂, —N(R)C(O)OR, —N(R)C(O)R, or —N(R)C(O)NR₂. In some embodiments, each instance of R^{CyAC} is independently —NR₂, —N(R)C(O)OR, or —N(R)C(O)R. In some embodiments, each instance of R^{CyAC} is independently —NR₂, —N(R)C(O)OR, —N(R)C(O)R, or —N(R)S(O)₂R.

In some embodiments, each instance of R^{CyAC} is independently an optionally substituted C₁₋₆ aliphatic. In some embodiments, each instance of R^{CyAC} is independently an optionally substituted phenyl. In some embodiments, each instance of R^{CyAC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{CyAC} is independently an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{CyAC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted C₁₋₆ aliphatic or an optionally substituted phenyl. In some embodiments, each instance of R^{CyAC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{CyAC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic. In some embodiments, R^{CyAC} is phenyl. In some embodiments, each instance of R^{CyAC} is independently a 3-7 membered saturated or partially unsaturated monocyclic heterocyclic ring having 1-2 heteroatoms independently selected from nitrogen, oxygen, and sulfur. In some embodiments, each instance of R^{CyAC} is independently a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{CyAC} is independently phenyl or a 5-6 membered monocyclic heteroaryl ring having 1-4 heteroatoms independently selected from nitrogen, oxygen, and sulfur.

In some embodiments, each instance of R^{CyAC} is independently a C₁₋₆ aliphatic or phenyl. In some embodiments, each instance of R^{CyAC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{CyAC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{CyAC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{CyAC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen atoms. In some embodiments, each instance of R^{CyAC} is independently oxo, deuterium, fluorine, chlorine, —CN, —OH, —OCH₃, —OCHF₂, —OCF₃, —CH₃, —CHF₂, or —CF₃.

In some embodiments, each instance of R^{CyAC} is independently halogen, —CN, —O-(optionally substituted C₁₋₆ aliphatic), or an optionally substituted C₁₋₆ aliphatic. In some embodiments, each instance of R^{CyAC} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{CyAC} is independently halogen or C₁₋₃ aliphatic optionally substituted with 1-3 halogen. In some embodiments, each instance of R^{CyAC} is independently fluorine, chlorine, —CH₃, —CHF₂, or —CF₃.

In some embodiments, each instance of R^{CyAC} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n is 0, 1, 2, 3, 4, or 5. In some embodiments, n is 0. In some embodiments, n is 1. In some embodiments, n is 2. In some embodiments, n is 3. In some embodiments, n is 4. In some embodiments, n is 5. In some embodiments, n is 0 or 1. In some embodiments, n is 0, 1, or 2. In some embodiments, n is 0, 1, 2, or 3. In some embodiments, n is 0, 1, 2, 3, or 4. In some embodiments, n is 1 or 2. In some embodiments, n is 1, 2, or 3. In some embodiments, n is 1, 2, 3, or 4. In some embodiments, n is 1, 2, 3, 4, or 5. In some embodiments, n is 2 or 3. In some embodiments, n is 2, 3, or 4. In some embodiments, n is 2, 3, 4, or 5. In some embodiments, n is 3 or 4. In some embodiments, n is 3, 4, or 5. In some embodiments, n is 4 or 5. In some embodiments, n is selected from the values represented in the compounds in Table 1.

As defined generally above, r¹ is 0, 1, 2, 3, 4, or 5. In some embodiments, r¹ is 0. In some embodiments, r¹ is 1. In some embodiments, r¹ is 2. In some embodiments, r¹ is 3. In some embodiments, r¹ is 4. In some embodiments, r¹ is 5. In some embodiments, r¹ is 0 or 1. In some embodiments, r¹ is 0, 1, or 2. In some embodiments, r¹ is 0, 1, 2, or 3. In some embodiments, r¹ is 0, 1, 2, 3, or 4. In some embodiments, r¹ is 1 or 2. In some embodiments, r¹ is 1, 2, or 3. In some embodiments, r¹ is 1, 2, 3, or 4. In some embodiments, r¹ is 1, 2, 3, 4, or 5. In some embodiments, r¹ is 2 or 3. In some embodiments, r¹ is 2, 3, or 4. In some embodiments, r¹ is 2, 3, 4, or 5. In some embodiments, r¹ is 3 or 4. In some embodiments, r¹ is 3, 4, or 5. In some embodiments, r¹ is 4 or 5. In some embodiments, r¹ is selected from the values represented in the compounds in Table 1.

As defined generally above, r² is 0, 1, 2, 3, 4, or 5. In some embodiments, r² is 0. In some embodiments, r² is 1. In some embodiments, r² is 2. In some embodiments, r² is 3. In some embodiments, r² is 4. In some embodiments, r² is 5. In some embodiments, r² is 0 or 1. In some embodiments, r² is 0, 1, or 2. In some embodiments, r² is 0, 1, 2, or 3. In some embodiments, r² is 0, 1, 2, 3, or 4. In some embodiments, r² is 1 or 2. In some embodiments, r² is 1, 2, or 3. In some embodiments, r² is 1, 2, 3, or 4. In some embodiments, r² is 1, 2, 3, 4, or 5. In some embodiments, r² is 2 or 3. In some embodiments, r² is 2, 3, or 4. In some embodiments, r² is 2, 3, 4, or 5. In some embodiments, r² is 3 or 4. In some embodiments, r² is 3, 4, or 5. In some embodiments, r² is 4 or 5. In some embodiments, r² is selected from the values represented in the compounds in Table 1.

As defined generally above, r³ is 0, 1, 2, 3, 4, or 5. In some embodiments, r³ is 0. In some embodiments, r³ is 1. In some embodiments, r³ is 2. In some embodiments, r³ is 3. In some embodiments, r³ is 4. In some embodiments, r³ is 5. In some embodiments, r³ is 0 or 1. In some embodiments, r³ is 0, 1, or 2. In some embodiments, r³ is 0, 1, 2, or 3. In some embodiments, r³ is 0, 1, 2, 3, or 4. In some embodiments, r³ is 1 or 2. In some embodiments, r³ is 1, 2, or 3. In some embodiments, r³ is 1, 2, 3, or 4. In some embodiments, r³ is 1, 2, 3, 4, or 5. In some embodiments, r³ is 2 or 3. In some embodiments, r³ is 2, 3, or 4. In some embodiments, r³ is 2, 3, 4, or 5. In some embodiments, r³ is 3 or 4. In some embodiments, r³ is 3, 4, or 5. In some embodiments, r³ is 4 or 5. In some embodiments, r³ is selected from the values represented in the compounds in Table 1.

As defined generally above, r⁴ is 0, 1, 2, 3, 4, or 5. In some embodiments, r⁴ is 0. In some embodiments, r⁴ is 1. In some embodiments, r⁴ is 2. In some embodiments, r⁴ is 3. In some embodiments, r⁴ is 4. In some embodiments, r⁴ is 5. In some embodiments, r⁴ is 0 or 1. In some embodiments, r⁴ is 0, 1, or 2. In some embodiments, r⁴ is 0, 1, 2, or 3. In some embodiments, r⁴ is 0, 1, 2, 3, or 4. In some embodiments, r⁴ is 1 or 2. In some embodiments, r⁴ is 1, 2, or 3. In some embodiments, r⁴ is 1, 2, 3, or 4. In some embodiments, r⁴ is 1, 2, 3, 4, or 5. In some embodiments, r⁴ is 2 or 3. In some embodiments, r⁴ is 2, 3, or 4. In some embodiments, r⁴ is 2, 3, 4, or 5. In some embodiments, r⁴ is 3 or 4. In some embodiments, r⁴ is 3, 4, or 5. In some embodiments, r⁴ is 4 or 5. In some embodiments, r⁴ is selected from the values represented in the compounds in Table 1.

As defined generally above, r⁵ is 0, 1, 2, 3, 4, or 5. In some embodiments, r⁵ is 0. In some embodiments, r⁵ is 1. In some embodiments, r⁵ is 2. In some embodiments, r⁵ is 3. In some embodiments, r⁵ is 4. In some embodiments, r⁵ is 5. In some embodiments, r⁵ is 0 or 1. In some embodiments, r⁵ is 0, 1, or 2. In some embodiments, r⁵ is 0, 1, 2, or 3. In some embodiments, r⁵ is 0, 1, 2, 3, or 4. In some embodiments, r⁵ is 1 or 2. In some embodiments, r⁵ is 1, 2, or 3. In some embodiments, r⁵ is 1, 2, 3, or 4. In some embodiments, r⁵ is 1, 2, 3, 4, or 5. In some embodiments, r⁵ is 2 or 3. In some embodiments, r⁵ is 2, 3, or 4. In some embodiments, r⁵ is 2, 3, 4, or 5. In some embodiments, r⁵ is 3 or 4. In some embodiments, r⁵ is 3, 4, or 5. In some embodiments, r⁵ is 4 or 5. In some embodiments, r⁵ is selected from the values represented in the compounds in Table 1.

As defined generally above, r⁶ is 0, 1, 2, 3, 4, or 5. In some embodiments, r⁶ is 0. In some embodiments, r⁶ is 1. In some embodiments, r⁶ is 2. In some embodiments, r⁶ is 3. In some embodiments, r⁶ is 4. In some embodiments, r⁶ is 5. In some embodiments, r⁶ is 0 or 1. In some embodiments, r⁶ is 0, 1, or 2. In some embodiments, r⁶ is 0, 1, 2, or 3. In some embodiments, r⁶ is 0, 1, 2, 3, or 4. In some embodiments, r⁶ is 1 or 2. In some embodiments, r⁶ is 1, 2, or 3. In some embodiments, r⁶ is 1, 2, 3, or 4. In some embodiments, r⁶ is 1, 2, 3, 4, or 5. In some embodiments, r⁶ is 2 or 3. In some embodiments, r⁶ is 2, 3, or 4. In some embodiments, r⁶ is 2, 3, 4, or 5. In some embodiments, r⁶ is 3 or 4. In some embodiments, r⁶ is 3, 4, or 5. In some embodiments, r⁶ is 4 or 5. In some embodiments, r⁶ is selected from the values represented in the compounds in Table 1.

In some embodiments, the present disclosure provides a compound of formula I-a, wherein Cy^{A} is selected from embodiments herein, forming a compound of formula I-a1, I-a2, I-a3, I-a4, I-a5, I-a6, I-a7, I-a8, I-a9, I-a10, I-a11, I-a12, I-a13, I-a14, I-a15, I-a16, I-a17, I-a18, I-a19, I-a20, I-a21, I-a22, I-a23, I-a24, or I-a25:

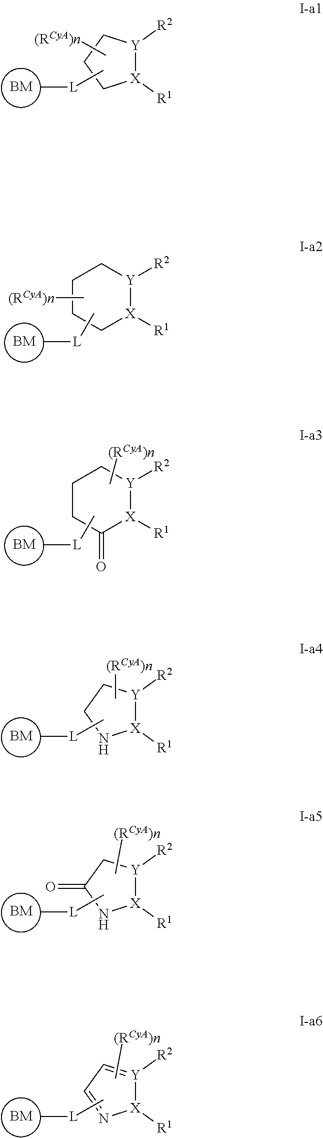

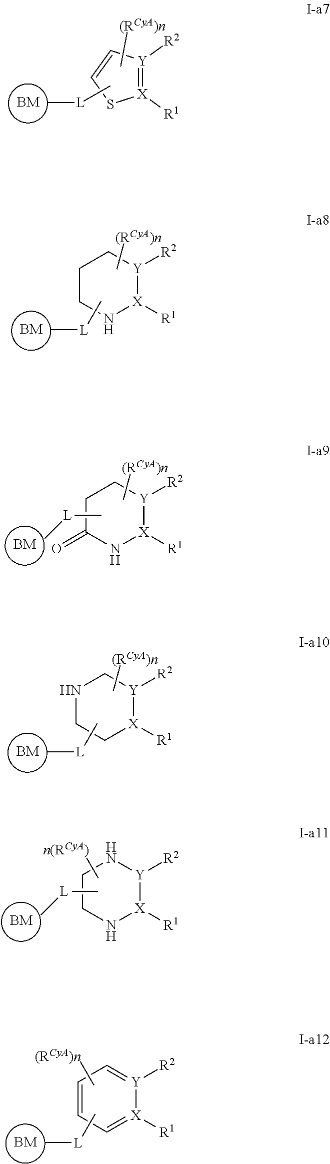

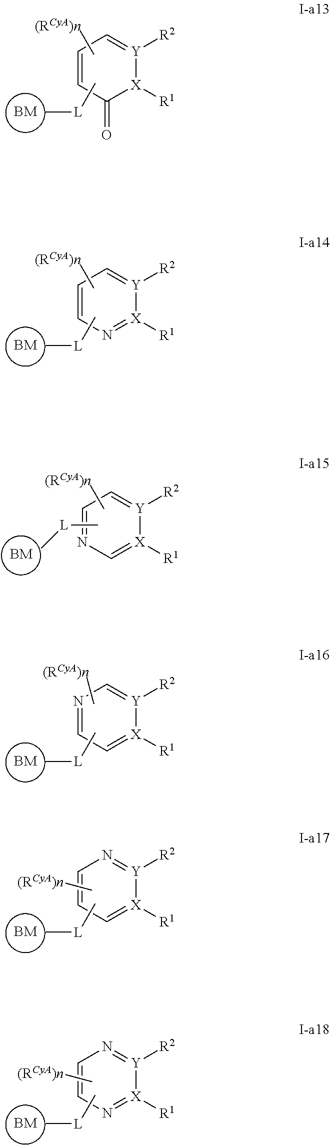

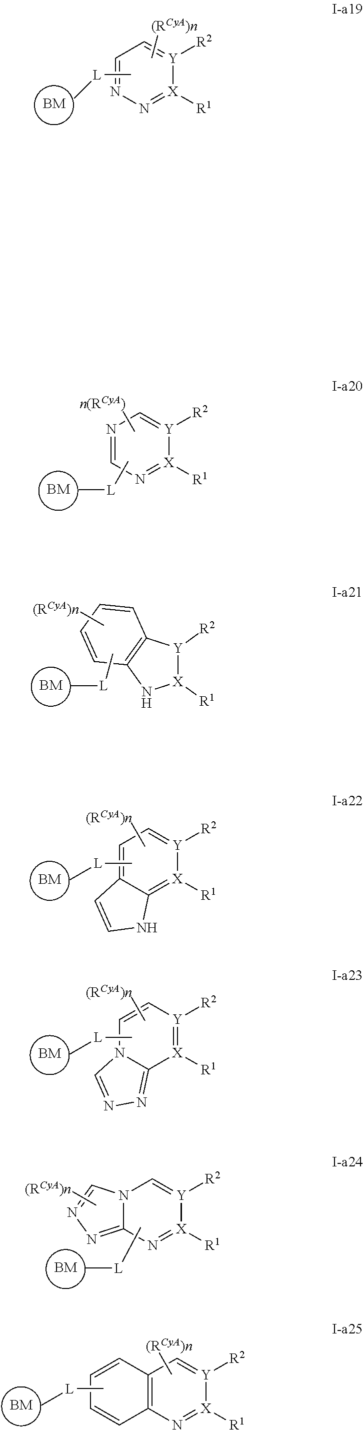

or a pharmaceutically acceptable salt thereof, wherein each of R¹, R², R^{CyA}, X, Y, L, BM, and n is as defined in embodiments and classes and subclasses herein.

In some embodiments, the present disclosure provides a compound of formula I-a12, I-a14, I-a15, I-a17, I-a16, I-a18, I-a19, I-a20, I-a22, I-a23, I-a24, and I-a25, wherein X is C and Y is C, forming a compound of formula I-aa1, I-aa2, I-aa3, I-aa4, I-aa4, I-aa5, I-aa6, I-aa7, I-aa8, I-aa9, I-aa10, I-aa11, or I-aa12:

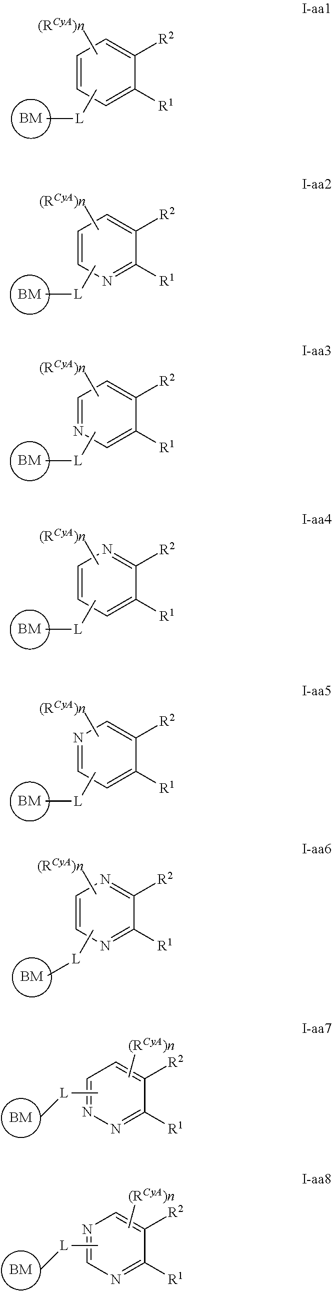

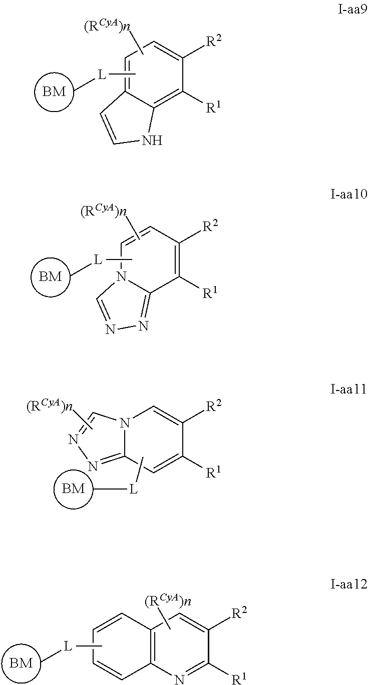

or a pharmaceutically acceptable salt thereof, wherein each of R¹, R², R^{CyA}, L, BM and n is as defined in embodiments and classes and subclasses herein.

In some embodiments, the present disclosure provides a compound of formula I-aa1 having the depicted point of attachment to -L-BM, forming a compound of formula I-aaa1, I-aaa2, or I-aaa3:

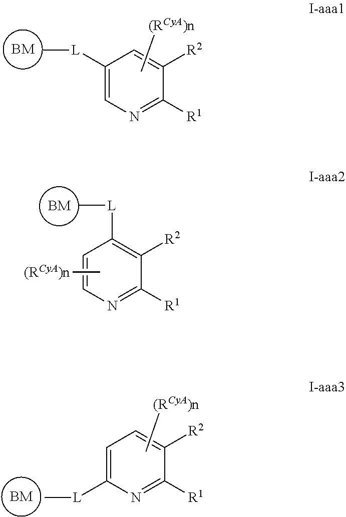

or a pharmaceutically acceptable salt thereof, wherein each of R¹, R², R^{CyA}, L, BM, and n is as defined in embodiments and classes and subclasses herein.

In certain embodiments, the present disclosure provides a compound of formula I, in which PIK is a PI3K binding moiety of formula I-b0, thereby forming a compound of formula I-b:

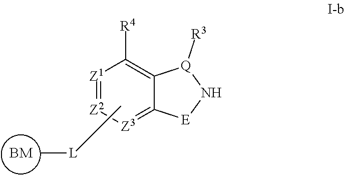

or a pharmaceutically acceptable salt thereof, wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
Q is CH, C(R^{Q}), or N;
Z¹ is CH, C(R^{Z1}), or N;
Z² is CH, C(R^{Z2}), or N;
Z³ is CH, C(R^{Z3}), or N;
R³ is -L³-R^{3A};
R⁴ is -L⁴-R^{4A};
each instance of R^{E} is independently H or -L^{E}-R^{EA};
R^{Q} is -L^{Q}-R^{QA};
R^{Z1} is -L^{Z1}-R^{Z1A};
R^{Z2} is -L^{Z2}-R^{Z2A};
R^{Z3} is -L^{Z3}-R^{Z3A}; or
   two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n¹ instances of R^{EEC};
   R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p¹ instances of R^{Q3C};
   R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C};
each of L³, L⁴, L^{E}, L^{Q}, L^{Z1}, L^{Z2}, and L^{Z3} is independently a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—. C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{3A} is R^{C} or R^{D} substituted by s¹ instances of R^{3C};
R^{4A} is R^{C} or R^{D} substituted by s² instances of R^{4C};
R^{EA} is R^{C} or R^{D} substituted by s³ instances of R^{EC};
R^{QA} is R^{C} or R^{D} substituted by s⁴ instances of R^{QC};
R^{Z1A} is R^{C} or R^{D} substituted by s⁵ instances of R^{Z1C};
R^{Z2A} is R^{C} or R^{D} substituted by s⁶ instances of R^{Z2C};
R^{Z3A} is R^{C} or R^{D} substituted by s⁷ instances of R^{Z3C};
R^{L1} is R^{C} or R^{D} substituted by s⁸ instances of R^{L1C};
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{D} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{3C}, R^{4C}, R^{EC}, R^{QC}, R^{Z1C}, R^{Z2C}, R^{Z3C}, R^{L1C}, R^{EEC}, R^{Q3C} and R^{Z2Z3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n¹, p¹, q¹, s¹, s², s³, s⁴, s⁵, s⁶, s⁷, and s⁸ is independently 0, 1, 2, 3, 4, or 5.

In some embodiments, the present disclosure provides a compound of Formula I-b, wherein each of E, Q, Z¹, Z², Z³, R³, R⁴, R^{E}, R^{Q}, R^{Z1}, R^{Z2}, R^{Z3}, L³, L⁴, L^{E}, L^{Q}, L^{Z1}, L^{Z2}, L^{Z3}, R^{3A}, R^{4A}, R^{EA}, R^{QA}, R^{Z1A}, R^{Z2A}, R^{Z3A}, R^{L}, R^{C}, R^{D}, R^{3C}, R^{4C}, R^{EC}, R^{QC}, R^{Z1C}, R^{Z2C}, R^{Z3C}, R^{L1C}, R^{EEC}, R^{Q3C}, R^{Z2Z3C}, R, n¹, p¹, q¹, s¹, s², s³, s⁴, s⁵, s⁶, s⁷, and s⁸ is as defined below, and described in embodiments herein, both singly and in combination.

As defined generally above, E is —C(O)—, —C(R^{E})₂—, —C(R^{E})₂C(R^{E})₂—, C₃₋₆ cycloalkylene, C₃₋₆ heterocycloalkylene, —C(S)—, —S(O)₂—, —OC(O)—, —N(R^{E})C(O)—, —C(O)N(R^{E})—, or —C(R^{E})₂C(O)—. In some embodiments, E is —C(O)—. In some embodiments, E is —OC(O)— or —N(R^{E})C(O)—. In some embodiments, E is —C(R^{E})₂—, C₃₋₆ cycloalkylene, or C₃₋₆ heterocycloalkylene.

In some embodiments, E is —C(O)—, —OC(O)—, —N(R^{E})C(O)—, or —C(R^{E})₂C(O)—. In some embodiments, E is —OC(O)—, —N(R^{E})C(O)—, or —C(R^{E})₂C(O)—. In some embodiments, E is —C(O)— or —N(R^{E})C(O)—.

In some embodiments, E is —C(O)—, —C(R^{E})₂—, —C(S)—, or —S(O)₂—. In some embodiments, E is —C(O)—, —C(R^{E})₂—, or —C(S)—. In some embodiments, E is —C(O)— or —C(S)—.

In some embodiments, E is —C(R^{E})₂C(R^{E})₂—, C₃₋₆ cycloalkylene, C₃₋₆ heterocycloalkylene, —OC(O)—, —N(R^{E})C(O)—, —C(O)N(R^{E})—, or —C(R^{E})₂C(O)—. In some embodiments, E is C₃₋₆ cycloalkylene or C₃₋₆ heterocycloalkylene. In some embodiments, E is —C(R^{E})₂C(R^{E})₂—, —OC(O)—, —N(R^{E})C(O)—, —C(O)N(R^{E})—, or —C(R^{E})₂C(O)—. In some embodiments, E is —OC(O)—, —N(R^{E})C(O)—, —C(O)N(R^{E})—, or —C(R^{E})₂C(O)—. In some embodiments, E is —OC(O)—, —N(R^{E})C(O)—, or —C(O)N(R^{E})—. In some embodiments, E is —N(R^{E})C(O)— or —C(O)N(R^{E})—. In some embodiments, E is —N(H)C(O)— or —C(O)N(H)—. In some embodiments, E is —N(CH₃)C(O)— or —C(O)N(CH₃)—.

In some embodiments, E is —S(O)₂—, —OC(O)—, —N(R^{E})C(O)—, or —C(O)N(R^{E})—. In some embodiments, E is —C(O)—, —C(R^{E})₂—, —C(R^{E})₂C(R^{E})₂—, C₃₋₆ cycloallylene, C₃₋₆ heterocycloalkylene, —C(S)—, or —C(R^{E})₂C(O)—. In some embodiments, E is —C(O)—, —C(R^{E})₂—, —C(R^{E})₂C(R^{E})₂—, —C(S)—, or —C(R^{E})₂C(O)—. In some embodiments, E is —C(O)—, —C(S)—, or —C(R^{E})₂C(O)—. In some embodiments, E is —C(R^{E})₂—, —C(R^{E})₂C(R^{E})₂—, or —C(R^{E})₂C(O)—. In some embodiments, E is —C(R^{E})₂— or —C(R^{E})₂C(R^{E})₂—.

In some embodiments, E is —C(R^{E})₂—. In some embodiments, E is —C(R^{E})₂C(R^{E})₂—. In some embodiments, E is C₃₋₆ cycloalkylene. In some embodiments, E is C₃₋₆ heterocycloalkylene. In some embodiments, E is —C(S)—. In some embodiments, E is —S(O)₂—. In some embodiments, E is —OC(O)—. In some embodiments, E is —N(R^{E})C(O)—. In some embodiments, E is —N(H)C(O)—. In some embodiments, E is —N(CH₃)C(O)—. In some embodiments, E is —C(O)N(R^{E})—. In some embodiments, E is —C(O)N(H)—. In some embodiments, E is —C(O)N(CH₃)—. In some embodiments, E is —C(R^{E})₂C(O)—.

In some embodiments, E is selected from the groups depicted in the compounds in Table 1.

As defined generally above, Q is CH, C(R^{Q}), or N. In some embodiments, Q is CH. In some embodiments, Q is C(R^{Q}). In some embodiments, Q is N. In some embodiments, Q is CH or C(R^{Q}). In some embodiments, Q is CH or N. In some embodiments, Q is C(R^{Q}) or N. In some embodiments, Q is selected from the groups depicted in the compounds in Table 1.

As defined generally above, Z¹ is CH, C(R^{Z1}), or N. In some embodiments, Z¹ is CH. In some embodiments, Z¹ is C(R^{Z1}). In some embodiments, Z¹ is N. In some embodiments, Z¹ is CH or C(R^{Z1}). In some embodiments, Z¹ is CH or N. In some embodiments, Z¹ is C(R^{Z1}) or N. In some embodiments, Z¹ is selected from the groups depicted in the compounds in Table 1.

As defined generally above, Z² is CH, C(R^{Z2}), or N. In some embodiments, Z² is CH. In some embodiments, Z² is C(R^{Z2}). In some embodiments, Z² is N. In some embodiments, Z² is CH or C(R^{Z2}). In some embodiments, Z² is CH or N. In some embodiments, Z² is C(R^{Z2}) or N. In some embodiments, Z² is selected from the groups depicted in the compounds in Table 1.

As defined generally above, Z³ is CH, C(R^{Z3}), or N. In some embodiments, Z³ is CH. In some embodiments, Z³ is C(R^{Z3}). In some embodiments, Z³ is N. In some embodiments, Z³ is CH or C(R^{Z3}). In some embodiments, Z³ is CH or N. In some embodiments, Z³ is C(R^{Z3}) or N. In some embodiments, Z³ is selected from the groups depicted in the compounds in Table 1.

As defined generally above, R³ is -L³-R^{3A} or R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p¹ instances of R^{Q1C}. In some embodiments, R³ is -L³-R^{3A}. In some embodiments, R³ is —R^{3A}.

In some embodiments, R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p¹ instances of R^{Q1C} In some embodiments, R^{Q} and R³ are taken together with their intervening atoms to form a 4-8 membered saturated or partially unsaturated monocyclic ring having 0-4 heteroatoms independently selected from nitrogen, oxygen, and sulfur; wherein said ring is substituted with p instances of R^{Q1C}. In some embodiments, R^{Q} and R³ are taken together with their intervening atoms to form an 8-12 membered saturated or partially unsaturated bicyclic ring having 0-4 heteroatoms independently selected from nitrogen, oxygen, and sulfur; wherein said ring is substituted with p instances of R^{Q1C}.

In some embodiments, R³ (i.e. -L³-R^{3A} taken together) is

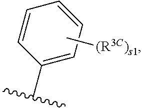

wherein R^{3C} and R³ are as defined in the embodiments and classes and subclasses herein. In some embodiments, R³ (i.e. -L³-R^{3A} taken together) is

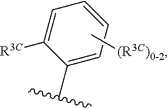

wherein R^{3C} is as defined in the embodiments and classes and subclasses herein. In some embodiments, R³ (i.e. -L³-R^{3A} taken together) is

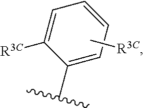

wherein R^{3C} is as defined in the embodiments and classes and subclasses herein. In some embodiments, R³ (i.e. -L³-R^{3A} taken together) is

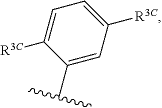

wherein R^{3C} is as defined in the embodiments and classes and subclasses herein. In some embodiments, R³ (i.e. -L³-R^{3A} taken together) is

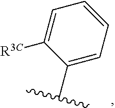

wherein R^{3C} is as defined in the embodiments and classes and subclasses herein.

In some embodiments, R³ (i.e. -L³-R^{3A} taken together) is

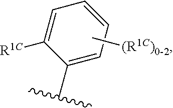

wherein each instance of R^{3C} is independently halogen, —CN, —O-(optionally substituted C₁₋₆ aliphatic), or an optionally substituted C₁₋₆ aliphatic. In some embodiments, R³ (i.e. -L³-R^{3A} taken together) is

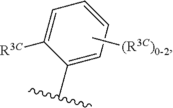

wherein each instance of R^{3C} is independently halogen or C₁₋₃ aliphatic optionally substituted with 1-3 halogen. In some embodiments, R³ (i.e. -L³-R^{3A} taken together) is

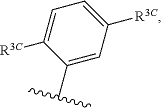

wherein each instance of R^{3C} is independently halogen or C₁₋₃ aliphatic optionally substituted with 1-3 halogen. In some embodiments, R³ (i.e. -L³-R^{3A} taken together) is

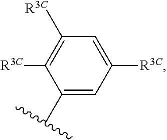

wherein each instance of R^{3C} is independently halogen or C₁₋₃ aliphatic optionally substituted with 1-3 halogen. In some embodiments, R³ (i.e. -L³-R^{3A} taken together) is

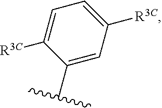

wherein each instance of R^{3C} is independently fluorine, chlorine, —CH₃, —CHF₂, or —CF₃. In some embodiments, R³ (i.e. -L³-R^{3A} taken together) is

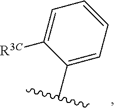

wherein R^{3C} is halogen or C₁₋₃ aliphatic optionally substituted with 1-3 halogen.

In some embodiments, R³ (i.e. -L³-R^{3A} taken together) is

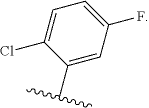 [SMILES: *c1cc(F)ccc1Cl]

In some embodiments, R³ (i.e. -L³-R^{3A} taken together) is

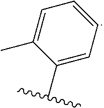 [SMILES: *c1ccccc1C]

In some embodiments, R³ (i.e. -L³-R^{3A} taken together) is

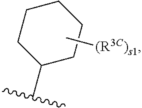

wherein R^{3C} and R³ are as defined in the embodiments and classes and subclasses herein. In some embodiments, R³ (i.e. -L³-R^{3A} taken together) is

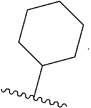 [SMILES: *C1CCCCC1]

In some embodiments, R³ (i.e. -L³-R^{3A} taken together) is

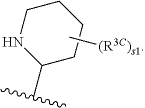

In some embodiments, R³ (i.e. -L³-R^{3A} taken together) is

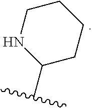 [SMILES: *C1CCCCN1]

In some embodiments, R³ is selected from the groups depicted in the compounds in Table 1.

As defined generally above, R⁴ is -L⁴-R^{4A}. In some embodiments, R⁴ (i.e. -L⁴-R^{4A} taken together) is —N(R)C(O)—R^{4A} or —R^{4A}, wherein R and R^{4A} are as defined in the embodiments and classes and subclasses herein. In some embodiments, R⁴ (i.e. -L⁴-R^{4A} taken together) is —N(R)C(O)—R^{4A}, wherein R and R^{4A} are as defined in the embodiments and classes and subclasses herein. In some embodiments, R⁴ (i.e. -L⁴-R^{4A} taken together) is —N(H)C(O)—R^{4A}, wherein R^{4A} is as defined in the embodiments and classes and subclasses herein. In some embodiments, R⁴ (i.e. -L⁴-R^{4A} taken together) is —N(H)C(O)—R^{4A}, wherein R^{4A} is R^{B} substituted by s instances of R^{4C}. In some embodiments, R⁴ is —R^{4A}.

In some embodiments, R⁴ is —N(H)C(O)—R^{4A}, —N(H)C(O)N(H)—R^{4A}, —C(O)N(H)—R^{4A}, —N(H)—R^{4A}, —S(O)₂CH₂—R^{4A}, —CH₂S(O)₂—R^{4A}, or —C(H)(CH₃)OH. In some embodiments, R⁴ is —N(H)C(O)—R^{4A}, —N(H)C(O)N(H)—R^{4A}, or —N(H)—R^{4A}. In some embodiments, R⁴ is —C(O)N(H)—R^{4A}, —CH₂S(O)₂—R^{4A}, or —C(H)(CH₃)OH. In some embodiments, R⁴ is —S(O)₂CH₂—R^{4A} or —CH₂S(O)₂—R^{4A}.

In some embodiments, R⁴ is —N(H)C(O)N(H)—R^{4A}. In some embodiments, R⁴ is —C(O)N(H)—R^{4A}. In some embodiments, R⁴ is —N(H)—R^{4A}. In some embodiments, R⁴ is —S(O)₂CH₂—R^{4A}. In some embodiments, R⁴ is —CH₂S(O)₂—R^{4A}. In some embodiments, R⁴ is —C(H)(CH₃)OH.

In some embodiments, R⁴ (i.e. -L⁴-R^{4A} taken together) is

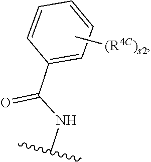

wherein R^{4C} and s² are as defined in the embodiments and classes and subclasses herein. In some embodiments, R⁴ (i.e. -L⁴-R^{4A} taken together) is

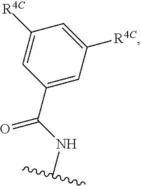

wherein R^{4C} is as defined in the embodiments and classes and subclasses herein.

In some embodiments, R⁴ (i.e. -L⁴-R^{4A} taken together) is

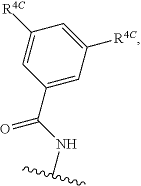

wherein each instance of R^{4C} is independently halogen, —CN, —O-(optionally substituted C₁₋₆ aliphatic), or an optionally substituted C₁₋₆ aliphatic. In some embodiments, R⁴ (i.e. -L⁴-R^{4A} taken together) is

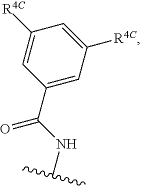

wherein each instance of R^{4C} is independently halogen or C₁₋₃ aliphatic optionally substituted with 1-3 halogen. In some embodiments, R⁴ (i.e. -L⁴-R^{4A} taken together) is

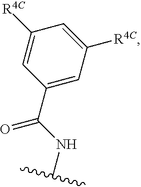

wherein each instance of R^{4C} is independently fluorine, chlorine, —CH₃, —CHF₂, or —CF₃. In some embodiments, R⁴ (i.e. -L⁴-R^{4A} taken together) is

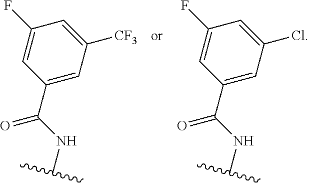 [SMILES: *NC(=O)c1cc(F)cc(C(F)(F)F)c1 ; *NC(=O)c1cc(F)cc(Cl)c1]

In some embodiments, R⁴ (i.e. -L⁴-R^{4A} taken together) is

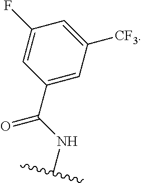 [SMILES: *NC(=O)c1cc(F)cc(C(F)(F)F)c1]

P In some embodiments, R⁴ (i.e. -L⁴-R^{4A} taken together) is

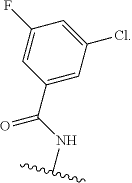 [SMILES: *NC(=O)c1cc(F)cc(Cl)c1]

In some embodiments, R⁴ (i.e. -L⁴-R^{4A} taken together) is

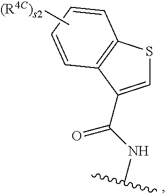

wherein R^{4C} and s² are as defined in the embodiments and classes and subclasses herein. In some embodiments, R⁴ (i.e. -L⁴-R^{4A} taken together) is

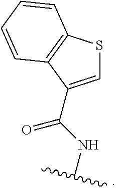 [SMILES: *NC(=O)c1csc2ccccc12]

In some embodiments, R⁴ (i.e. -L⁴-R^{4A} taken together) is

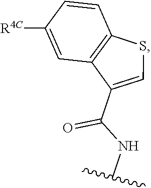

wherein R^{4C} is as defined in the embodiments and classes and subclasses herein.

In some embodiments, R⁴ (i.e. -L⁴-R^{4A} taken together) is

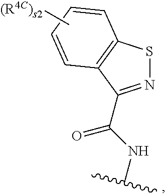

wherein R^{4C} and s² are as defined in the embodiments and classes and subclasses herein. In some embodiments, R⁴ (i.e. -L⁴-R^{4A} taken together) is

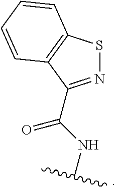 [SMILES: *NC(=O)c1nsc2ccccc12]

In some embodiments, R⁴ (i.e. -L⁴-R^{4A} taken together) is

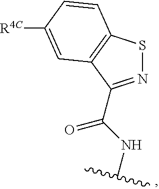

wherein R^{4C} is as defined in the embodiments and classes and subclasses herein.

In some embodiments, R⁴ (i.e. -L⁴-R^{4A} taken together) is

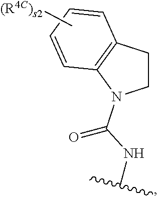

wherein R^{4C} and s² are as defined in the embodiments and classes and subclasses herein. In some embodiments, R⁴ (i.e. -L⁴-R^{4A} taken together) is

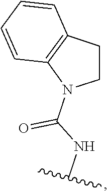 [SMILES: *NC(=O)N1CCc2ccccc21]

In some embodiments, R⁴ (i.e. -L⁴-R^{4A} taken together) is

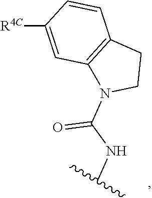

wherein R^{4C} is as defined in the embodiments and classes and subclasses herein.

In some embodiments, R⁴ (i.e. -L⁴-R^{4A} taken together) is

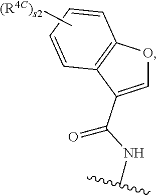

wherein R^{4C} and s² are as defined in the embodiments and classes and subclasses herein. In some embodiments, R⁴ (i.e. -L⁴-R^{4A} taken together) is

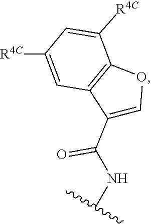

wherein R^{4C} is as defined in the embodiments and classes and subclasses herein.

In some embodiments, R⁴ (i.e. -L⁴-R^{4A} taken together) is

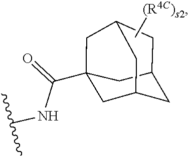

wherein R^{4C} and s² are as defined in the embodiments and classes and subclasses herein. In some embodiments, R⁴ (i.e. -L⁴-R^{4A} taken together) is

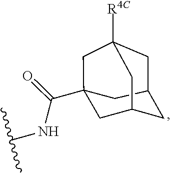

wherein R^{4C} is as defined in the embodiments and classes and subclasses herein.

In some embodiments, R⁴ (i.e. -L⁴-R^{4A} taken together) is

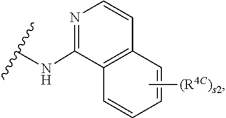

wherein R^{4C} and s² are as defined in the embodiments and classes and subclasses herein. In some embodiments, R⁴ (i.e. -L⁴-R^{4A} taken together) is

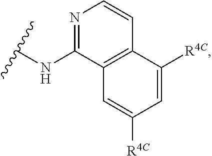

wherein R^{4C} is as defined in the embodiments and classes and subclasses herein.

In some embodiments, R⁴ (i.e. -L⁴-R^{4A} taken together) is

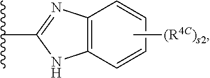

wherein R^{4C} and s² are as defined in the embodiments and classes and subclasses herein. In some embodiments, R⁴ (i.e. -L⁴-R^{4A} taken together) is

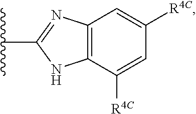

wherein R^{4C} is as defined in the embodiments and classes and subclasses herein.

In some embodiments, R⁴ is

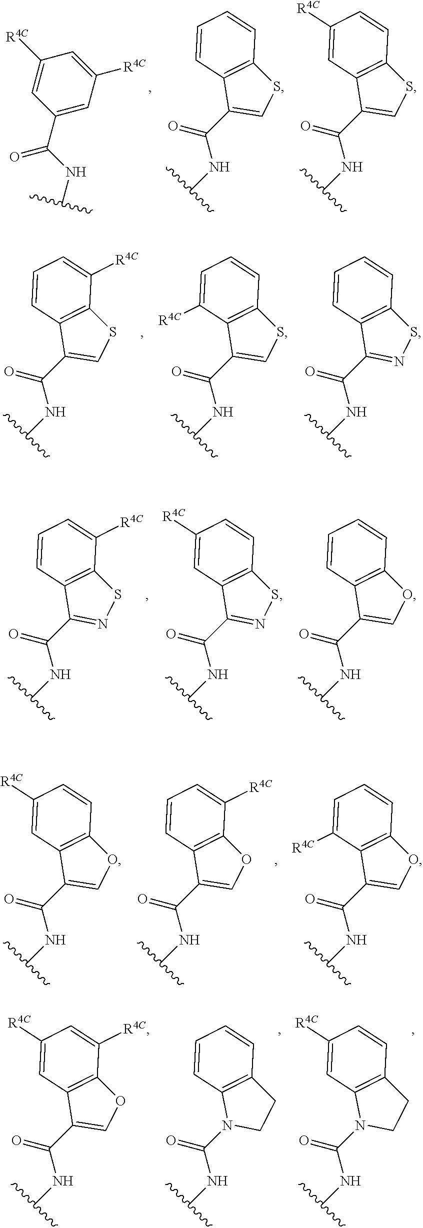

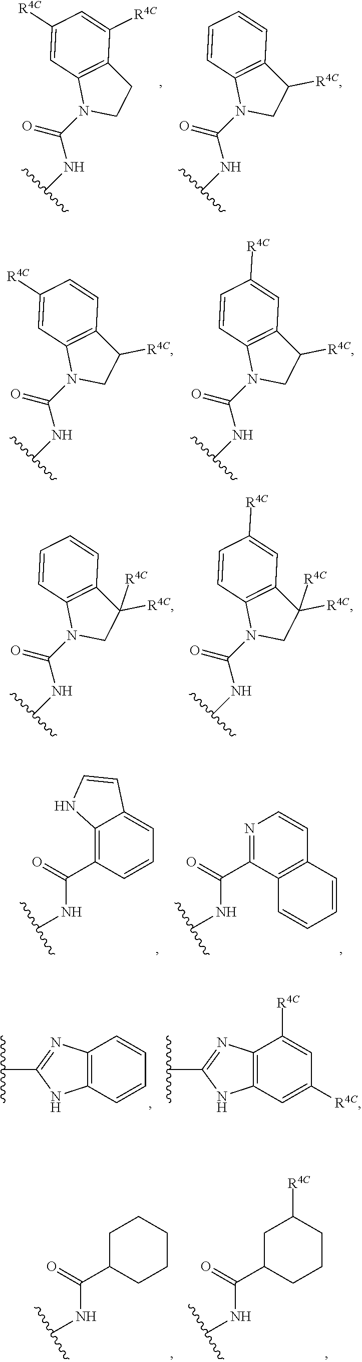

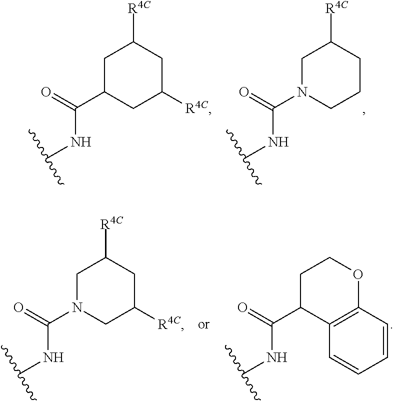

In some embodiments, R⁴ is

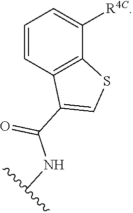

In some embodiments, R⁴ is

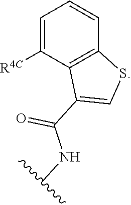

In some embodiments, R⁴ is

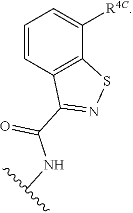

some embodiments, R⁴ is

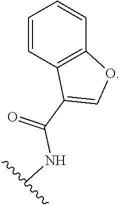 [SMILES: *NC(=O)c1coc2ccccc12]

In some embodiments, R⁴ is

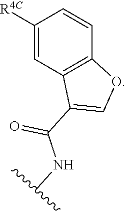

In some embodiments, R⁴ is

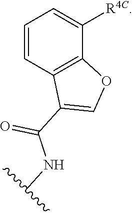

K in some embodiments, R⁴ is

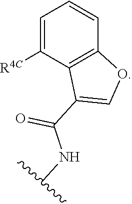

In some embodiments, R⁴ is

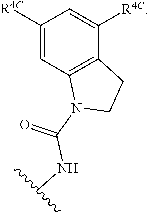

In some embodiments, R⁴ is

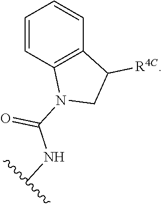

In some embodiments, R⁴ is

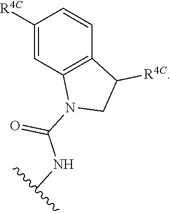

In some embodiments, R⁴ is

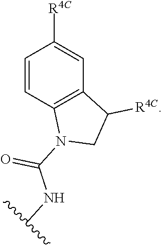

In some embodiments, R⁴ is

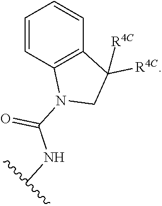

In some embodiments, R⁴ is

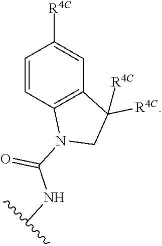

In some embodiments, R⁴ is

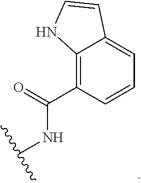 [SMILES: *NC(=O)c1cccc2cc[nH]c12]

In some embodiments, R⁴ is

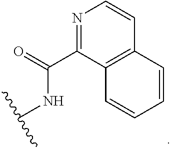 [SMILES: *NC(=O)c1nccc2ccccc12]

In some embodiments, R⁴ is

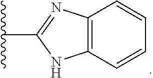 [SMILES: *c1nc2ccccc2[nH]1]

In some embodiments, R⁴ is

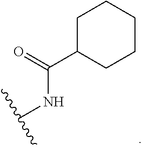 [SMILES: *NC(=O)C1CCCCC1]

In some embodiments, R⁴ is

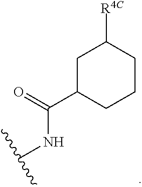

In some embodiments, R⁴ is

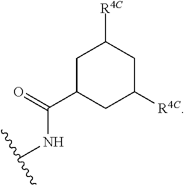

In some embodiments, R⁴ is

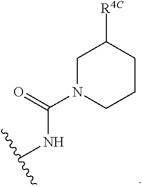

In some embodiments, R⁴ is

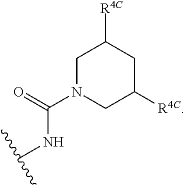

In some embodiments, R⁴ is

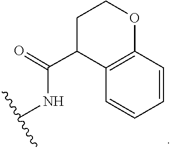 [SMILES: *NC(=O)C1CCOc2ccccc21]

In some embodiments, R⁴ is selected from the groups depicted in the compounds in Table 1.

As defined generally above, each instance of R^{E} is independently H or -L^{E}-R^{EA}; or two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n¹ instances of R^{EEC}.

In some embodiments, each instance of R^{E} is independently H or -L^{E}-R^{EA}. In some embodiments, R^{E} is H. In some embodiments, each instance of R^{E} is independently -L^{E}-R^{EA}In some embodiments, each instance of R^{E} is independently R^{EA}. In some embodiments, each instance of R^{E} is independently R^{A}. In some embodiments, each instance of R^{E} is independently R^{B} substituted by s³ instances of R^{EC}.

In some embodiments, each instance of R^{E} is independently H or C₁₋₆ aliphatic substituted by s³ instances of R^{EC}. In some embodiments, each instance of R^{E} is independently H or C₁₋₃ aliphatic substituted by s³ instances of R^{EC}. In some embodiments, each instance of R^{E} is independently H or C₁₋₃ aliphatic substituted by s³ instances of halogen. In some embodiments, each instance of R^{E} is independently H or C₁₋₃ aliphatic. In some embodiments, each instance of R^{E} is independently H, —CH₃, —CH₂F, —CHF₂—, or —CF₃. In some embodiments, each instance of R^{E} is independently H or —CH₃.

In some embodiments, each instance of R^{E} is independently C₁₋₆ aliphatic substituted by s³ instances of R^{EC}. In some embodiments, each instance of R^{E} is independently C₁₋₃ aliphatic substituted by s³ instances of R^{EC}. In some embodiments, each instance of R^{E} is independently C₁₋₃ aliphatic substituted by s³ instances of halogen. In some embodiments, each instance of R^{E} is independently C₁₋₃ aliphatic. In some embodiments, each instance of R^{E} is independently —CH₃, —CH₂F, —CHF₂—, or —CF₃. In some embodiments, R^{E} is —CH₃.

In some embodiments, two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EEC}In some embodiments, two instances of R^{E} are taken together with their intervening atoms to form a 3-8 membered saturated or partially unsaturated monocyclic ring having 0-4 heteroatoms independently selected from nitrogen, oxygen, and sulfur; wherein said ring is substituted with n instances of R^{EEC}. In some embodiments, two instances of R^{E} are taken together with their intervening atoms to form an 8-12 membered saturated or partially unsaturated bicyclic ring having 0-4 heteroatoms independently selected from nitrogen, oxygen, and sulfur; wherein said ring is substituted with n instances of R^{EE}C.

In some embodiments, R^{E} is selected from the groups depicted in the compounds in Table 1.

As defined generally above, R^{Q} is -L^{Q}-R^{QA} or R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p instances of R^{Q1C}. In some embodiments, R^{Q} is -L^{Q}-R^{QA}. In some embodiments, R^{Q} is —R^{QA}.

In some embodiments, R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p instances of R^{Q1C} In some embodiments, R^{Q} and R³ are taken together with their intervening atoms to form a 4-8 membered saturated or partially unsaturated monocyclic ring having 0-4 heteroatoms independently selected from nitrogen, oxygen, and sulfur; wherein said ring is substituted with p¹ instances of R^{Q1C}. In some embodiments, R^{Q} and R¹ are taken together with their intervening atoms to form an 8-12 membered saturated or partially unsaturated bicyclic ring having 0-4 heteroatoms independently selected from nitrogen, oxygen, and sulfur; wherein said ring is substituted with p¹ instances of R^{Q1C}.

In some embodiments, R^{Q} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Q} is halogen, —CN, —OH, —O-(optionally substituted C₁₋₆ aliphatic), or an optionally substituted C₁₋₆ aliphatic. In some embodiments, R^{Q} is halogen, —OH, or C₁₋₃ aliphatic optionally substituted with 1-3 halogen. In some embodiments, R^{Q} is fluorine, chlorine, —OH, or —CH₃. In some embodiments, R^{Q} is deuterium. In some embodiments, R^{Q} is selected from the groups depicted in the compounds in Table 1.

As defined generally above, R^{Z1} is -L^{Z1}-R^{Z1A}. In some embodiments, R^{Z1} is —R^{Z1A}. In some embodiments, R^{Z1} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z1} is halogen, —CN, —OH, —O-(optionally substituted C₁₋₆ aliphatic), or an optionally substituted C₁₋₆ aliphatic. In some embodiments, R^{Z1} is halogen, —OH, —O—(C₁₋₃ aliphatic), or C₁₋₃ aliphatic, wherein each C₁₋₃ aliphatic is optionally substituted with 1-3 halogen. In some embodiments, R^{Z1} is fluorine, chlorine, —OCH₃, or —CH₃. In some embodiments, R^{Z1} is selected from the groups depicted in the compounds in Table 1.

As defined generally above, R^{Z2} is -L^{Z2}-R^{Z2A} or R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2ZC}. In some embodiments, R^{Z2} is -L^{Z2}-R^{Z2A}. In some embodiments, R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C}. In some embodiments, R^{Z2} is selected from the groups depicted in the compounds in Table 1.

As defined generally above, R^{Z3} is -L^{Z3}-R^{Z3A} or R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C}. In some embodiments, R^{Z3} is -L^{Z3}-R^{Z3A}. In some embodiments, R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C}. In some embodiments, R^{Z3} is selected from the groups depicted in the compounds in Table 1.

As defined generally above, L³ is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L³ is a covalent bond. In some embodiments, L³ is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L³ is a C₁₋₄ bivalent saturated or unsaturated, straight or branched hydrocarbon chain.

In some embodiments, L³ is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L³ is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L³ is a C₁₋₂ bivalent saturated or unsaturated hydrocarbon chain. In some embodiments, L³ is selected from the groups depicted in the compounds in Table 1.

As defined generally above, L⁴ is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁴ is a covalent bond. In some embodiments, L⁴ is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁴ is a C₁₋₄ bivalent saturated or unsaturated, straight or branched hydrocarbon chain.

In some embodiments, L⁴ is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁴ is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L⁴ is a C₁₋₂ bivalent saturated or unsaturated hydrocarbon chain.

In some embodiments, L⁴ is —N(R)C(O)— or —N(R)C(O)N(R)—. In some embodiments, L⁴ is —N(H)C(O)— or —N(H)C(O)N(H)—. In some embodiments, L⁴ is —N(R)C(O)—. In some embodiments, L⁴ is —N(H)C(O)—. In some embodiments, L⁴ is —N(R)C(O)N(R)—. In some embodiments, L⁴ is —N(H)C(O)N(H)—. In some embodiments, L⁴ is —N(R)—. In some embodiments, L⁴ is —N(H)—. In some embodiments, L⁴ is a covalent bond. In some embodiments, L⁴ is selected from the groups depicted in the compounds in Table 1.

As defined generally above, L^{E} is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a covalent bond. In some embodiments, L^{E} is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a bivalent saturated or unsaturated, straight or branched hydrocarbon chain.

In some embodiments, L^{E} is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{E} is a C₁₋₂ bivalent saturated or unsaturated hydrocarbon chain. In some embodiments, L^{E} is selected from the groups depicted in the compounds in Table 1.

As defined generally above, L^{Q} is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a covalent bond. In some embodiments, L^{Q} is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₄ bivalent saturated or unsaturated, straight or branched hydrocarbon chain.

In some embodiments, L^{Q} is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Q} is a C₁₋₂ bivalent saturated or unsaturated hydrocarbon chain. In some embodiments, L^{Q} is selected from the groups depicted in the compounds in Table 1.

As defined generally above, L^{Z1} is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z1} is a covalent bond. In some embodiments, L^{Z1} is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z1} is a C₁₋₄ bivalent saturated or unsaturated, straight or branched hydrocarbon chain.

In some embodiments, L^{Z1} is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z1} is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Z1} is a C₁₋₂ bivalent saturated or unsaturated hydrocarbon chain. In some embodiments, L^{Z1} is selected from the groups depicted in the compounds in Table 1.

As defined generally above, L^{Z2} is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z2} is a covalent bond. In some embodiments, L^{Z2} is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z2} is a C₁₋₄ bivalent saturated or unsaturated, straight or branched hydrocarbon chain.

In some embodiments, L^{Z2} is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z2} is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Z2} is a C₁₋₂ bivalent saturated or unsaturated hydrocarbon chain.

In some embodiments, L^{Z2} is —C(H)₂—, —CH(R^{L1})—, —C(R^{L1})₂—, C₃₋₆ cycloallylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is —C(H)₂—, —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Z2} is —C(H)₂—, —N(R)—, —N(R)C(O)—, or —C(O)N(R)—. In some embodiments, L^{Z2} is —C(H)₂—, —N(H)—, —N(H)C(O)—, or —C(O)N(H)—. In some embodiments, L^{Z2} is selected from the groups depicted in the compounds in Table 1.

As defined generally above, L^{Z3} is a covalent bond, or a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z3} is a covalent bond. In some embodiments, L^{Z3} is a C₁₋₄ bivalent saturated or unsaturated, straight or branched hydrocarbon chain wherein one or two methylene units of the chain are optionally and independently replaced by —CH(R^{L1})—, —C(R^{L1})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z3} is a C₁₋₄ bivalent saturated or unsaturated, straight or branched hydrocarbon chain.

In some embodiments, L^{Z3} is a C₁₋₂ bivalent saturated or unsaturated hydrocarbon chain wherein one or two methylene units of the chain are optionally and independently replaced by —CH(R^{L1})—, —C(R^{L1})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z3} is a C₁₋₂ bivalent saturated or unsaturated hydrocarbon chain wherein one or two methylene units of the chain are optionally and independently replaced by —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Z3} is a C₁₋₂ bivalent saturated or unsaturated hydrocarbon chain.

In some embodiments, L^{Z3} is —C(H)₂—, —CH(R^{L1})—, —C(R^{L1})₂—, C₃₋₆ cycloallylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Z3} is —C(H)₂—, —CH(R^{L1})—, —C(R^{L1})₂—, —N(R)—, —N(R)C(O)—, —C(O)N(R)—, —N(R)S(O)₂—, —S(O)₂N(R)—, or —O—. In some embodiments, L^{Z3} is —C(H)₂—, —N(R)—, —N(R)C(O)—, or —C(O)N(R)—. In some embodiments, L^{Z3} is —C(H)₂—, —N(H)—, —N(H)C(O)—, or —C(O)N(H)—. In some embodiments, L^{Z3} is selected from the groups depicted in the compounds in Table 1.

As defined generally above, R^{3A} is R^{C} or R^{D} substituted by s instances of R^{3C}. In some embodiments, R^{3A} is R^{C}. In some embodiments, R^{3A} is R^{D} substituted by s instances of R^{3C}.

In some embodiments, R^{3A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{3A} is substituted by s¹ instances of R^{3C}.

In some embodiments, R^{3A} is phenyl substituted by s¹ instances of R^{3C}. In some embodiments, R^{3A} is an 8-10 membered bicyclic heteroaryl ring having 1-4 heteroatoms independently selected from nitrogen, oxygen, and sulfur, wherein R^{3A} is substituted by s¹ instances of R^{3C}. In some embodiments, R^{3A} is phenyl or an 8-10 membered bicyclic heteroaryl ring having 1-4 heteroatoms independently selected from nitrogen, oxygen, and sulfur, wherein R^{3A} is substituted by s¹ instances of R^{3C}.

In some embodiments, R^{3A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{3A} is substituted by s¹ instances of R^{3C}.

In some embodiments, R^{3A} is phenyl substituted by s¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{3A} is an 8-10 membered bicyclic heteroaryl ring having 1-4 heteroatoms independently selected from nitrogen, oxygen, and sulfur, wherein R^{3A} is substituted by s¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{3A} is phenyl or an 8-10 membered bicyclic heteroaryl ring having 1-4 heteroatoms independently selected from nitrogen, oxygen, and sulfur, wherein R^{3A} is substituted by s¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{3A} is phenyl substituted by 1-3 instances of R^{3C}. In some embodiments, R^{3A} is phenyl substituted by 2 instances of R^{3C}. In some embodiments, R^{3A} is phenyl substituted by 1 instance of R^{3C}.

In some embodiments, R^{3A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{3A} is phenyl substituted by 1-3 instances of a group independently selected from halogen and C₁₋₃ aliphatic optionally substituted with 1-3 halogen. In some embodiments, R^{3A} is phenyl substituted by 1-3 instances of a group independently selected from fluorine, chlorine, —CH₃, —CHF₂, and —CF₃.

In some embodiments, R^{3A} is phenyl substituted by 2 instances of a group independently selected from halogen, —CN, —O-(optionally substituted C₁₋₆ aliphatic), and an optionally substituted C₁₋₆ aliphatic. In some embodiments, R^{3A} is phenyl substituted by 2 instances of a group independently selected from halogen and C₁₋₃ aliphatic optionally substituted with 1-3 halogen. In some embodiments, R^{3A} is phenyl substituted by 2 instances of a group independently selected from fluorine, chlorine, —CH₃, —CHF₂, and —CF₃.

In some embodiments, R^{3A} is phenyl substituted by one group selected from halogen, —CN, —O-(optionally substituted C₁₋₆ aliphatic), and an optionally substituted C₁₋₆ aliphatic. In some embodiments, R^{3A} is phenyl substituted by one halogen or C₁₋₃ aliphatic group optionally substituted with 1-3 halogen. In some embodiments, R^{3A} is phenyl substituted by one fluorine, chlorine, —CH₃, —CHF₂, or —CF₃.

In some embodiments, R^{3A} is

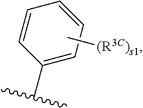

wherein R^{3C} and s¹ are as defined in the embodiments and classes and subclasses herein. In some embodiments, R^{3A} is

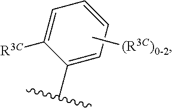

wherein R^{3C} is as defined in the embodiments and classes and subclasses herein. In some embodiments, R^{3A} is

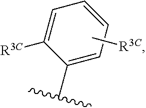

wherein R^{3C} is as defined in the embodiments and classes and subclasses herein. In some embodiments, R^{3A} is

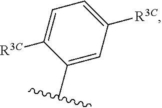

wherein R^{3C} is as defined in the embodiments and classes and subclasses herein. In some embodiments, R^{3A} is

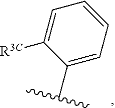

wherein R^{3C} is as defined in the embodiments and classes and subclasses herein.

In some embodiments, R^{3A} is

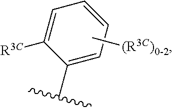

wherein each instance of R^{3C} is independently halogen, —CN, —O-(optionally substituted C₁₋₆ aliphatic), or an optionally substituted C₁₋₆ aliphatic. In some embodiments, R^{3A} is

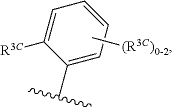

wherein each instance of R^{3C} is independently halogen or C₁₋₃ aliphatic optionally substituted with 1-3 halogen. In some embodiments, R^{3A} is

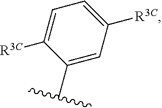

wherein each instance of R^{3C} is independently halogen or C₁₋₃ aliphatic optionally substituted with 1-3 halogen. In some embodiments, R^{3A} is

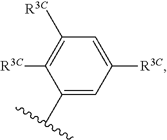

wherein each instance of R^{3C} is independently halogen or C₁₋₃ aliphatic optionally substituted with 1-3 halogen. In some embodiments, R^{3A} is

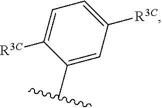

wherein each instance of R^{3C} is independently fluorine, chlorine, —CH₃, —CHF₂, or —CF₃. In some embodiments, R^{3A} is

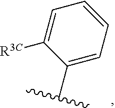

, wherein R^{3C} is halogen or C₁₋₃ aliphatic optionally substituted with 1-3 halogen.

In some embodiments, R^{3A} is

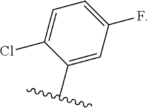 [SMILES: *c1cc(F)ccc1Cl]

. In some embodiments, R^{3A} is

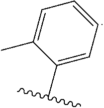 [SMILES: *c1ccccc1C]

In some embodiments, R^{3A} is

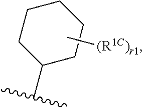

wherein R^{3C} and s¹ are as defined in the embodiments and classes and subclasses herein. In some embodiments, R^{3A} is

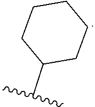 [SMILES: *C1CCCCC1]

In some embodiments, R^{3A} is

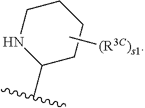

In some embodiments, R^{3A} is

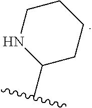 [SMILES: *C1CCCCN1]

In some embodiments, R^{3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{3A} is oxo. In some embodiments, R^{3A} is halogen. In some embodiments, R^{3A} is —CN. In some embodiments, R^{3A} is —NO₂. In some embodiments, R^{3A} is —OR. In some embodiments, R^{3A} is —SR. In some embodiments, R^{3A} is —NR₂. In some embodiments, R^{3A} is —S(O)₂R. In some embodiments, R^{3A} is —S(O)₂NR₂. In some embodiments, R^{3A} is —S(O)₂F. In some embodiments, R^{3A} is —S(O)R. In some embodiments, R^{3A} is —S(O)NR₂. In some embodiments, R^{3A} is —S(O)(NR)R. In some embodiments, R^{3A} is —C(O)R. In some embodiments, R^{3A} is —C(O)OR. In some embodiments, R^{3A} is —C(O)NR₂. In some embodiments, R^{3A} is —C(O)N(R)OR. In some embodiments, R^{3A} is —OC(O)R. In some embodiments, R^{3A} is —OC(O)NR₂. In some embodiments, R^{3A} is —N(R)C(O)OR. In some embodiments, R^{3A} is —N(R)C(O)R. In some embodiments, R^{3A} is —N(R)C(O)NR₂. In some embodiments, R^{3A} is —N(R)C(NR)NR₂. In some embodiments, R^{3A} is —N(R)S(O)₂NR₂. In some embodiments, R^{3A} is —N(R)S(O)₂R. In some embodiments, R^{3A} is —P(O)R₂. In some embodiments, R^{3A} is —P(O)(R)OR. In some embodiments, R^{3A} is —B(OR)₂. In some embodiments, R^{3A} is deuterium.

In some embodiments, R^{3A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{3A} is halogen, —CN, or —NO₂. In some embodiments, R^{3A} is —OR, —SR, or —NR₂. In some embodiments, R^{3A} is —S(O)₂R, —S(O)₂NR₂, —S(O)₂F, —S(O)R, —S(O)NR₂, or —S(O)(NR)R. In some embodiments, R^{3A} is —C(O)R, —C(O)OR, —C(O)NR₂, or —C(O)N(R)OR. In some embodiments, R^{3A} is —OC(O)R or —OC(O)NR₂. In some embodiments, R^{3A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{3A} is —P(O)R₂ or —P(O)(R)OR.

In some embodiments, R^{3A} is —OR, —OC(O)R, or —OC(O)NR₂. In some embodiments, R^{3A} is —SR, —S(O)₂R, —S(O)₂NR₂, —S(O)₂F, —S(O)R, —S(O)NR₂, or —S(O)(NR)R. In some embodiments, R^{3A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{3A} is —S(O)₂R, —S(O)₂NR₂, or —S(O)₂F. In some embodiments, R^{3A} is —S(O)R, —S(O)NR₂, or —S(O)(NR)R. In some embodiments, R^{3A} is —SR, —S(O)₂R, or —S(O)R. In some embodiments, R^{3A} is —S(O)₂NR₂, —S(O)NR₂, or —S(O)(NR)R. In some embodiments, R^{3A} is —S(O)₂NR₂ or —S(O)NR₂. In some embodiments, R^{3A} is —SR, —S(O)₂R, —S(O)₂NR₂, or —S(O)R.

In some embodiments, R^{3A} is —N(R)C(O)OR, —N(R)C(O)R, or —N(R)C(O)NR₂. In some embodiments, R^{3A} is —N(R)S(O)₂NR₂ or —N(R)S(O)₂R. In some embodiments, R^{3A} is —N(R)C(O)OR or —N(R)C(O)R. In some embodiments, R^{3A} is —N(R)C(O)NR₂ or —N(R)S(O)₂NR₂. In some embodiments, R^{3A} is —N(R)C(O)OR, —N(R)C(O)R, or —N(R)S(O)₂R.

In some embodiments, R^{3A} is —NR₂, —N(R)C(O)OR, —N(R)C(O)R, or —N(R)C(O)NR₂. In some embodiments, R^{3A} is —NR₂, —N(R)C(O)OR, or —N(R)C(O)R. In some embodiments, R^{3A} is —NR₂, —N(R)C(O)OR, —N(R)C(O)R, or —N(R)S(O)₂R.

In some embodiments, R^{3A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is a C₁₋₆ aliphatic chain substituted by s¹ instances of R^{3C} In some embodiments, R^{3A} is phenyl substituted by s¹ instances of R^{3C}. In some embodiments, R^{3A} is naphthyl substituted by s¹ instances of R^{3C}. In some embodiments, R^{3A} is a 5-6 membered monocyclic heteroaryl ring having 1-4 heteroatoms independently selected from nitrogen, oxygen, and sulfur; wherein said ring is substituted by s¹ instances of R^{3C}. In some embodiments, R^{3A} is an 8-10 membered bicyclic heteroaryl ring having 1-4 heteroatoms independently selected from nitrogen, oxygen, and sulfur; wherein said ring is substituted by s¹ instances of R^{3C}. In some embodiments, R^{3A} is a 3-7 membered saturated or partially unsaturated monocyclic carbocyclic ring substituted by s¹ instances of R^{3C}. In some embodiments, R^{3A} is a 5-12 membered saturated or partially unsaturated bicyclic carbocyclic ring substituted by s¹ instances of R^{3C}. In some embodiments, R^{3A} is a 3-7 membered saturated or partially unsaturated monocyclic heterocyclic ring having 1-2 heteroatoms independently selected from nitrogen, oxygen, and sulfur; wherein said ring is substituted by s¹ instances of R^{3C}. In some embodiments, R^{3A} is a 7-12 membered saturated or partially unsaturated bicyclic heterocyclic ring having 1-4 heteroatoms independently selected from nitrogen, oxygen, and sulfur; wherein said ring is substituted by s¹ instances of R^{3C}.

In some embodiments, R^{3A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s¹ instances of R^{3C}. In some embodiments, R^{3A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is phenyl; naphthyl; a 3-7 membered saturated or partially unsaturated monocyclic carbocyclic ring; or a 5-12 membered saturated or partially unsaturated bicyclic carbocyclic ring; each of which is substituted by s¹ instances of R^{3C}. In some embodiments, R^{3A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s¹ instances of R^{3C}. In some embodiments, R^{3A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is phenyl or naphthyl; each of which is substituted by s¹ instances of R^{3C}. In some embodiments, R^{3A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s¹ instances of R^{3C}. In some embodiments, R^{3A} is a 3-7 membered saturated or partially unsaturated monocyclic carbocyclic ring or a 5-12 membered saturated or partially unsaturated bicyclic carbocyclic ring; each of which is substituted by s¹ instances of R^{3C}. In some embodiments, R^{3A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is phenyl or a 5-6 membered monocyclic heteroaryl ring having 1-4 heteroatoms independently selected from nitrogen, oxygen, and sulfur; each of which is substituted by s¹ instances of R^{3C}. In some embodiments, R^{3A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s¹ instances of R^{3C}. In some embodiments, R^{3A} is naphthyl or an 8-10 membered bicyclic heteroaryl ring having 1-4 heteroatoms independently selected from nitrogen, oxygen, and sulfur; each of which is substituted by s¹ instances of R^{3C}. In some embodiments, R^{3A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is phenyl or a 3-7 membered saturated or partially unsaturated monocyclic carbocyclic ring; each of which is substituted by s¹ instances of R^{3C} In some embodiments, R^{3A} is naphthyl or a 5-12 membered saturated or partially unsaturated bicyclic carbocyclic ring; each of which is substituted by s¹ instances of R^{3C}. In some embodiments, R^{3A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s¹ instances of R^{3C}. In some embodiments, R^{3A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}.

In some embodiments, R^{3A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s¹ instances of R^{3C}. In some embodiments, R^{3A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s¹ instances of R^{3C}. In some embodiments, R^{3A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s¹ instances of R^{3C}.

In some embodiments, R^{3A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s¹ instances of R^{3C}. In some embodiments, R^{3A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s¹ instances of R^{3C}. In some embodiments, R^{3A} is a C₁₋₆ aliphatic chain, phenyl, or a 3-7 membered saturated or partially unsaturated monocyclic carbocyclic ring; each of which is substituted by s¹ instances of R^{3C}.

In some embodiments, R^{3A} is selected from the groups depicted in the compounds in Table 1.

As defined generally above, R^{4A} is R^{C} or R^{D} substituted by s² instances of R^{4C}. In some embodiments, R^{4A} is R^{C}. In some embodiments, R^{4A} is R^{D} substituted by s² instances of R^{4C}.

In some embodiments, R^{4A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of R^{4C}.

In some embodiments, R^{4A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of R^{4C}. In some embodiments, R^{4A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of R^{4C}. In some embodiments, R^{4A} is phenyl or an 8-10 membered bicyclic heteroaryl ring having 1-4 heteroatoms independently selected from nitrogen, oxygen, and sulfur; wherein R^{4A} is substituted by s² instances of R^{4C}.

In some embodiments, R^{4A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{4A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{4A} is phenyl or an 8-10 membered bicyclic heteroaryl ring having 1-4 heteroatoms independently selected from nitrogen, oxygen, and sulfur; wherein R^{4A} is substituted by s² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{4A} is phenyl substituted by s² instances of R^{4C}. In some embodiments, R^{4A} is phenyl substituted by s² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{4A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{4A} is phenyl substituted by 1-3 instances of a group independently selected from halogen and C₁₋₃ aliphatic optionally substituted with 1-3 halogen. In some embodiments, R^{4A} is phenyl substituted by 1-3 instances of a group independently selected from fluorine, chlorine, —CH₃, —CHF₂, and —CF₃.

In some embodiments, R^{4A} is phenyl substituted by 2 instances of a group independently selected from halogen, —CN, —O-(optionally substituted C₁₋₆ aliphatic), and an optionally substituted C₁₋₆ aliphatic. In some embodiments, R^{4A} is phenyl substituted by 2 instances of a group independently selected from halogen and C₁₋₃ aliphatic optionally substituted with 1-3 halogen. In some embodiments, R^{4A} is phenyl substituted by 2 instances of a group independently selected from fluorine, chlorine, —CH₃, —CHF₂, and —CF₃.

In some embodiments, R^{4A} is an 8-10 membered bicyclic heteroaryl ring having 1-4 heteroatoms independently selected from nitrogen, oxygen, and sulfur; wherein R^{4A} is substituted by s² instances of R^{4C}. In some embodiments, R^{4A} is an 8-10 membered bicyclic heteroaryl ring having 1-4 heteroatoms independently selected from nitrogen, oxygen, and sulfur; wherein R^{4A} is substituted by 2 instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{4A} is an 8-10 membered bicyclic heteroaryl ring having 1-2 heteroatoms independently selected from nitrogen, oxygen, and sulfur; wherein R^{4A} is substituted by s² instances of R^{4C}. In some embodiments, R^{4A} is an 8-10 membered bicyclic heteroaryl ring having 1-2 heteroatoms independently selected from nitrogen, oxygen, and sulfur; wherein R^{4A} is substituted by 2 instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{4A} is an 8-10 membered bicyclic heteroaryl ring having 1-2 heteroatoms independently selected from nitrogen, oxygen, and sulfur; wherein R^{4A} is substituted by 0-2 instances of a group independently selected from halogen, —CN, —O-(optionally substituted C₁₋₆ aliphatic), and an optionally substituted C₁₋₆ aliphatic. In some embodiments, R^{4A} is an 8-10 membered bicyclic heteroaryl ring having 1-2 heteroatoms independently selected from nitrogen, oxygen, and sulfur; wherein R^{4A} is substituted by 0-2 instances of a group independently selected from halogen and C₁₋₃ aliphatic optionally substituted with 1-3 halogen. In some embodiments, R^{4A} is an 8-10 membered bicyclic heteroaryl ring having 1-2 heteroatoms independently selected from nitrogen, oxygen, and sulfur; wherein R^{4A} is substituted by 0-2 instances of a group independently selected from fluorine, chlorine, —CH₃, —CHF₂, and —CF₃.

In some embodiments, R^{4A} is:

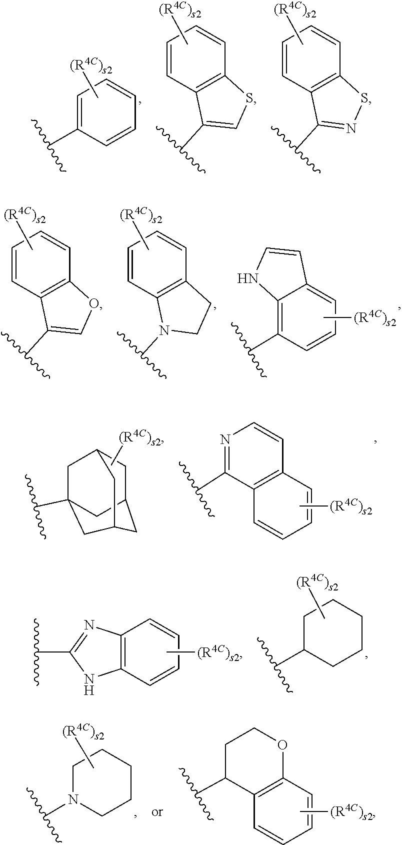

wherein R^{4C} and s² are as defined in the embodiments and classes and subclasses herein. In some embodiments, R^{4A} is

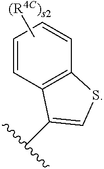

In some embodiments, R^{4A} is

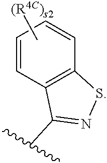

In some embodiments, R^{4A} is

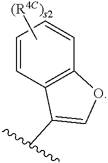

In some embodiments, R^{4A} is

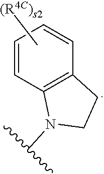

In some embodiments, R^{4A} is

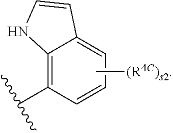

In some embodiments, R^{4A} is

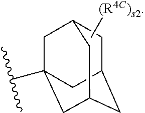

In some embodiments R^{4A} is

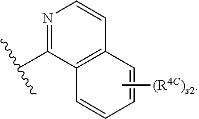

In some embodiments, R^{4A} is

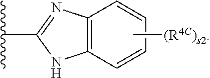

In some embodiments, R^{4A} is

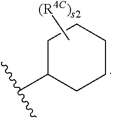

In some embodiments, R^{4A} is

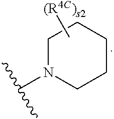

In some embodiments, R^{4A} is

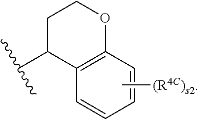

In some embodiments, R^{4A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{4A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{4A} is oxo. In some embodiments, R^{4A} is halogen. In some embodiments, R^{4A} is —CN. In some embodiments, R^{4A} is —NO₂. In some embodiments, R^{4A} is —OR. In some embodiments, R^{4A} is —SR. In some embodiments, R^{4A} is —NR₂. In some embodiments, R^{4A} is —S(O)₂R. In some embodiments, R^{4A} is —S(O)₂NR₂. In some embodiments, R^{4A} is —S(O)₂F. In some embodiments, R^{4A} is —S(O)R. In some embodiments, R^{4A} is —S(O)NR₂. In some embodiments, R^{4A} is —S(O)(NR)R. In some embodiments, R^{4A} is —C(O)R. In some embodiments, R^{4A} is —C(O)OR. In some embodiments, R^{4A} is —C(O)NR₂. In some embodiments, R^{4A} is —C(O)N(R)OR. In some embodiments, R^{4A} is —OC(O)R. In some embodiments, R^{4A} is —OC(O)NR₂. In some embodiments, R^{4A} is —N(R)C(O)OR. In some embodiments, R^{4A} is —N(R)C(O)R. In some embodiments, R^{4A} is —N(R)C(O)NR₂. In some embodiments, R^{4A} is —N(R)C(NR)NR₂. In some embodiments, R^{4A} is —N(R)S(O)₂NR₂. In some embodiments, R^{4A} is —N(R)S(O)₂R. In some embodiments, R^{4A} is —P(O)R₂. In some embodiments, R^{4A} is —P(O)(R)OR. In some embodiments, R^{4A} is —B(OR)₂. In some embodiments, R^{4A} is deuterium.

In some embodiments, R^{4A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{4A} is halogen, —CN, or —NO₂. In some embodiments, R^{4A} is —OR, —SR, or —NR₂. In some embodiments, R^{4A} is —S(O)₂R, —S(O)₂NR₂, —S(O)₂F, —S(O)R, —S(O)NR₂, or —S(O)(NR)R. In some embodiments, R^{4A} is —C(O)R, —C(O)OR, —C(O)NR₂, or —C(O)N(R)OR. In some embodiments, R^{4A} is —OC(O)R or —OC(O)NR₂. In some embodiments, R^{4A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{4A} is —P(O)R₂ or —P(O)(R)OR.

In some embodiments, R^{4A} is —OR, —OC(O)R, or —OC(O)NR₂. In some embodiments, R^{4A} is —SR, —S(O)₂R, —S(O)₂NR₂, —S(O)₂F, —S(O)R, —S(O)NR₂, or —S(O)(NR)R. In some embodiments, R^{4A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{4A} is —S(O)₂R, —S(O)₂NR₂, or —S(O)₂F. In some embodiments, R^{4A} is —S(O)R, —S(O)NR₂, or —S(O)(NR)R. In some embodiments, R^{4A} is —SR, —S(O)₂R, or —S(O)R. In some embodiments, R^{4A} is —S(O)₂NR₂, —S(O)NR₂, or —S(O)(NR)R. In some embodiments, R^{4A} is —S(O)₂NR₂ or —S(O)NR₂. In some embodiments, R^{4A} is —SR, —S(O)₂R, —S(O)₂NR₂, or —S(O)R.

In some embodiments, R^{4A} is —N(R)C(O)OR, —N(R)C(O)R, or —N(R)C(O)NR₂. In some embodiments, R^{4A} is —N(R)S(O)₂NR₂ or —N(R)S(O)₂R. In some embodiments, R^{4A} is —N(R)C(O)OR or —N(R)C(O)R. In some embodiments, R^{4A} is —N(R)C(O)NR₂ or —N(R)S(O)₂NR₂. In some embodiments, R^{4A} is —N(R)C(O)OR, —N(R)C(O)R, or —N(R)S(O)₂R.

In some embodiments, R^{4A} is —NR₂, —N(R)C(O)OR, —N(R)C(O)R, or —N(R)C(O)NR₂. In some embodiments, R^{4A} is —NR₂, —N(R)C(O)OR, or —N(R)C(O)R. In some embodiments, R^{4A} is —NR₂, —N(R)C(O)OR, —N(R)C(O)R, or —N(R)S(O)₂R.

In some embodiments, R^{4A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is a C₁₋₆ aliphatic chain substituted by s² instances of R^{4C} In some embodiments, R^{4A} is phenyl substituted by s² instances of R^{4C}. In some embodiments, R^{4A} is naphthyl substituted by s² instances of R^{4C}. In some embodiments, R^{4A} is cubanyl substituted by s² instances of R^{4C}. In some embodiments, R^{4A} is adamantyl substituted by s² instances of R^{4C}. In some embodiments, R^{4A} is a 5-6 membered monocyclic heteroaryl ring having 1-4 heteroatoms independently selected from nitrogen, oxygen, and sulfur; wherein said ring is substituted by s² instances of R^{4C}. In some embodiments, R^{4A} is an 8-10 membered bicyclic heteroaryl ring having 1-4 heteroatoms independently selected from nitrogen, oxygen, and sulfur; wherein said ring is substituted by s² instances of R^{4C}. In some embodiments, R^{4A} is a 3-7 membered saturated or partially unsaturated monocyclic carbocyclic ring substituted by s² instances of R^{4C}. In some embodiments, R^{4A} is a 5-12 membered saturated or partially unsaturated bicyclic carbocyclic ring substituted by s² instances of R^{4C}. In some embodiments, R^{4A} is a 3-7 membered saturated or partially unsaturated monocyclic heterocyclic ring having 1-2 heteroatoms independently selected from nitrogen, oxygen, and sulfur; wherein said ring is substituted by s² instances of R^{4C}. In some embodiments, R^{4A} is a 7-12 membered saturated or partially unsaturated bicyclic heterocyclic ring having 1-4 heteroatoms independently selected from nitrogen, oxygen, and sulfur; wherein said ring is substituted by s² instances of R^{4C}.

In some embodiments, R^{4A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s² instances of R^{4C}. In some embodiments, R^{4A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by s² instances of R^{4C}. In some embodiments, R^{4A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s² instances of R^{4C}. In some embodiments, R^{4A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is phenyl or naphthyl; each of which is substituted by s² instances of R^{4C}. In some embodiments, R^{4A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s² instances of R^{4C}. In some embodiments, R^{4A} is a 3-7 membered saturated or partially unsaturated monocyclic carbocyclic ring or a 5-12 membered saturated or partially unsaturated bicyclic carbocyclic ring; each of which is substituted by s² instances of R^{4C}. In some embodiments, R^{4A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is phenyl or a 5-6 membered monocyclic heteroaryl ring having 1-4 heteroatoms independently selected from nitrogen, oxygen, and sulfur; each of which is substituted by s² instances of R^{4C}. In some embodiments, R^{4A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s² instances of R^{4C}. In some embodiments, R^{4A} is naphthyl or an 8-10 membered bicyclic heteroaryl ring having 1-4 heteroatoms independently selected from nitrogen, oxygen, and sulfur; each of which is substituted by s² instances of R^{4C}. In some embodiments, R^{4A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is phenyl or a 3-7 membered saturated or partially unsaturated monocyclic carbocyclic ring; each of which is substituted by s² instances of R^{4C} In some embodiments, R^{4A} is naphthyl; cubanyl; adamantyl; or a 5-12 membered saturated or partially unsaturated bicyclic carbocyclic ring; each of which is substituted by s² instances of R^{4C}. In some embodiments, R^{4A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s² instances of R^{4C}. In some embodiments, R^{4A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}.

In some embodiments, R^{4A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s² instances of R^{4C}. In some embodiments, R^{4A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by s² instances of R^{4C}. In some embodiments, R^{4A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s² instances of R^{4C}.

In some embodiments, R^{4A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by s² instances of R^{4C}. In some embodiments, R^{4A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s² instances of R^{4C}. In some embodiments, R^{4A} is a C₁₋₆ aliphatic chain, phenyl, or a 3-7 membered saturated or partially unsaturated monocyclic carbocyclic ring; each of which is substituted by s² instances of R^{4C}.

In some embodiments, R^{4A} is selected from the groups depicted in the compounds in Table 1.

As defined generally above, R^{EA} is R^{C} or R^{D} substituted by s³ instances of R^{EC} In some embodiments, R^{EA} is R^{C}. In some embodiments, R^{EA} is R^{D} substituted by s³ instances of R^{EC}.

In some embodiments, each instance of R^{EA} is independently C₁₋₆ aliphatic substituted by s³ instances of R^{EC}. In some embodiments, each instance of R^{EA} is independently C₁₋₃ aliphatic substituted by s³ instances of R^{EC}. In some embodiments, each instance of R^{EA} is independently C₁₋₃ aliphatic substituted by s³ instances of halogen. In some embodiments, each instance of R^{EA} is independently C₁₋₃ aliphatic. In some embodiments, each instance of R^{EA} is independently —CH₃, —CH₂F, —CHF₂—, or —CF₃. In some embodiments, R^{EA} is —CH₃.

In some embodiments, R^{EA} is selected from the groups depicted in the compounds in Table 1.

As defined generally above, R^{QA} is R^{C} or R^{D} substituted by s⁴ instances of R^{QC}. In some embodiments, R^{QA} is R^{C}. In some embodiments, R^{QA} is R^{D} substituted by s⁴ instances of R^{QC}.

In some embodiments, R^{QA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{QA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{QA} is oxo. In some embodiments, R^{QA} is halogen. In some embodiments, R^{QA} is —CN. In some embodiments, R^{QA} is —NO₂. In some embodiments, R^{QA} is —OR. In some embodiments, R^{QA} is —SR. In some embodiments, R^{QA} is —NR₂. In some embodiments, R^{QA} is —S(O)₂R. In some embodiments, R^{QA} is —S(O)₂NR₂. In some embodiments, R^{QA} is —S(O)₂F. In some embodiments, R^{QA} is —S(O)R. In some embodiments, R^{QA} is —S(O)NR₂. In some embodiments, R^{QA} is —S(O)(NR)R. In some embodiments, R^{QA}is —C(O)R. In some embodiments, R^{QA} is —C(O)OR. In some embodiments, R^{QA} is —C(O)NR₂. In some embodiments, R^{QA} is —C(O)N(R)OR. In some embodiments, R^{QA} is —OC(O)R. In some embodiments, R^{QA} is —OC(O)NR₂. In some embodiments, R^{QA} is —N(R)C(O)OR. In some embodiments, R^{QA} is —N(R)C(O)R. In some embodiments, R^{QA} is —N(R)C(O)NR₂. In some embodiments, R^{QA} is —N(R)C(NR)NR₂. In some embodiments, R^{QA} is —N(R)S(O)₂NR₂. In some embodiments, R^{QA} is —N(R)S(O)₂R. In some embodiments, R^{QA} is —P(O)R₂. In some embodiments, R^{QA} is —P(O)(R)OR. In some embodiments, R^{QA} is —B(OR)₂. In some embodiments, R^{QA} is deuterium.

In some embodiments, R^{QA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{QA} is halogen, —CN, or —NO₂. In some embodiments, R^{QA} is —OR, —SR, or —NR₂. In some embodiments, R^{QA} is —S(O)₂R, —S(O)₂NR₂, —S(O)₂F, —S(O)R, —S(O)NR₂, or —S(O)(NR)R. In some embodiments, R^{QA} is —C(O)R, —C(O)OR, —C(O)NR₂, or —C(O)N(R)OR. In some embodiments, R^{QA} is —OC(O)R or —OC(O)NR₂. In some embodiments, R^{QA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{QA} is —P(O)R₂ or —P(O)(R)OR.

In some embodiments, R^{QA} is —OR, —OC(O)R, or —OC(O)NR₂. In some embodiments, R^{QA} is —SR, —S(O)₂R, —S(O)₂NR₂, —S(O)₂F, —S(O)R, —S(O)NR₂, or —S(O)(NR)R. In some embodiments, R^{QA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{QA} is —S(O)₂R, —S(O)₂NR₂, or —S(O)₂F. In some embodiments, R^{QA} is —S(O)R, —S(O)NR₂, or —S(O)(NR)R. In some embodiments, R^{QA} is —SR, —S(O)₂R, or —S(O)R. In some embodiments, R^{QA} is —S(O)₂NR₂, —S(O)NR₂, or —S(O)(NR)R. In some embodiments, R^{QA} is —S(O)₂NR₂ or —S(O)NR₂. In some embodiments, R^{QA} is —SR, —S(O)₂R, —S(O)₂NR₂, or —S(O)R.

In some embodiments, R^{QA} is —N(R)C(O)OR, —N(R)C(O)R, or —N(R)C(O)NR₂. In some embodiments, R^{QA} is —N(R)S(O)₂NR₂ or —N(R)S(O)₂R. In some embodiments, R^{QA} is —N(R)C(O)OR or —N(R)C(O)R. In some embodiments, R^{QA} is —N(R)C(O)NR₂ or —N(R)S(O)₂NR₂. In some embodiments, R^{QA} is —N(R)C(O)OR, —N(R)C(O)R, or —N(R)S(O)₂R.

In some embodiments, R^{QA} is —NR₂, —N(R)C(O)OR, —N(R)C(O)R, or —N(R)C(O)NR₂. In some embodiments, R^{QA} is —NR₂, —N(R)C(O)OR, or —N(R)C(O)R. In some embodiments, R^{QA} is —NR₂, —N(R)C(O)OR, —N(R)C(O)R, or —N(R)S(O)₂R.

In some embodiments, R^{QA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is a C₁₋₆ aliphatic chain substituted by s⁴ instances of R^{QC}. In some embodiments, R^{QA} is phenyl substituted by s⁴ instances of R^{QC}. In some embodiments, R^{QA} is naphthyl substituted by s⁴ instances of R^{QC}. In some embodiments, R^{QA} is a 5-6 membered monocyclic heteroaryl ring having 1-4 heteroatoms independently selected from nitrogen, oxygen, and sulfur; wherein said ring is substituted by s⁴ instances of R^{QC}. In some embodiments, R^{QA} is an 8-10 membered bicyclic heteroaryl ring having 1-4 heteroatoms independently selected from nitrogen, oxygen, and sulfur; wherein said ring is substituted by s⁴ instances of R^{QC}. In some embodiments, R^{QA} is a 3-7 membered saturated or partially unsaturated monocyclic carbocyclic ring substituted by s⁴ instances of R^{QC}. In some embodiments, R^{QA} is a 5-12 membered saturated or partially unsaturated bicyclic carbocyclic ring substituted by s⁴ instances of R^{QC}. In some embodiments, R^{QA} is a 3-7 membered saturated or partially unsaturated monocyclic heterocyclic ring having 1-2 heteroatoms independently selected from nitrogen, oxygen, and sulfur; wherein said ring is substituted by s⁴ instances of R^{QC}. In some embodiments, R^{QA} is a 7-12 membered saturated or partially unsaturated bicyclic heterocyclic ring having 1-4 heteroatoms independently selected from nitrogen, oxygen, and sulfur; wherein said ring is substituted by s⁴ instances of R^{QC}.

In some embodiments, R^{QA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁴ instances of R^{QC}. In some embodiments, R^{QA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl; naphthyl; a 3-7 membered saturated or partially unsaturated monocyclic carbocyclic ring; or a 5-12 membered saturated or partially unsaturated bicyclic carbocyclic ring; each of which is substituted by s⁴ instances of R^{QC}. In some embodiments, R^{QA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁴ instances of R^{QC}. In some embodiments, R^{QA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl or naphthyl; each of which is substituted by s⁴ instances of R^{QC}. In some embodiments, R^{QA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁴ instances of R^{QC}. In some embodiments, R^{QA} is a 3-7 membered saturated or partially unsaturated monocyclic carbocyclic ring or a 5-12 membered saturated or partially unsaturated bicyclic carbocyclic ring; each of which is substituted by s⁴ instances of R^{QC}. In some embodiments, R^{QA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl or a 5-6 membered monocyclic heteroaryl ring having 1-4 heteroatoms independently selected from nitrogen, oxygen, and sulfur; each of which is substituted by s⁴ instances of R^{QC}. In some embodiments, R^{QA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s⁴ instances of R^{QC}. In some embodiments, R^{QA} is naphthyl or an 8-10 membered bicyclic heteroaryl ring having 1-4 heteroatoms independently selected from nitrogen, oxygen, and sulfur; each of which is substituted by s⁴ instances of R^{QC}. In some embodiments, R^{QA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is phenyl or a 3-7 membered saturated or partially unsaturated monocyclic carbocyclic ring; each of which is substituted by s⁴ instances of R^{QC}. In some embodiments, R^{QA} is naphthyl or a 5-12 membered saturated or partially unsaturated bicyclic carbocyclic ring; each of which is substituted by s⁴ instances of R^{QC}. In some embodiments, R^{QA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁴ instances of R^{QC}. In some embodiments, R^{QA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}.

In some embodiments, R^{QA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁴ instances of R^{QC}. In some embodiments, R^{QA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s⁴ instances of R^{QC}. In some embodiments, R^{QA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁴ instances of R^{QC}.

In some embodiments, R^{QA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s⁴ instances of R^{QC}. In some embodiments, R^{QA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s⁴ instances of R^{QC}. In some embodiments, R^{QA} is a C₁₋₆ aliphatic chain, phenyl, or a 3-7 membered saturated or partially unsaturated monocyclic carbocyclic ring; each of which is substituted by s⁴ instances of R^{QC}.

In some embodiments, R^{QA} is selected from the groups depicted in the compounds in Table 1.

As defined generally above, R^{Z1A} is R^{C} or R^{D} substituted by s⁵ instances of R^{Z1C}. In some embodiments, R^{Z1A} is R^{C}. In some embodiments, R^{Z1A} is R^{D} substituted by s⁵ instances of R^{Z1C}.

In some embodiments, R^{Z1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{Z1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z1A} is oxo. In some embodiments, R^{Z1A} is halogen. In some embodiments, R^{Z1A} is —CN. In some embodiments, R^{Z1A} is —NO₂. In some embodiments, R^{Z1A} is —OR. In some embodiments, R^{Z1A} is —SR. In some embodiments, R^{Z1A} is —NR₂. In some embodiments, R^{Z1A} is —S(O)₂R. In some embodiments, R^{Z1A} is —S(O)₂NR₂. In some embodiments, R^{Z1A} is —S(O)₂F. In some embodiments, R^{Z1A} is —S(O)R. In some embodiments, R^{Z1A} is —S(O)NR₂. In some embodiments, R^{Z1A} is —S(O)(NR)R. In some embodiments, R^{Z1A} is —C(O)R. In some embodiments, R^{Z1A} is —C(O)OR. In some embodiments, R^{Z1A} is —C(O)NR₂. In some embodiments, R^{Z1A} is —C(O)N(R)OR. In some embodiments, R^{Z1A} is —OC(O)R. In some embodiments, R^{Z1A} is —OC(O)NR₂. In some embodiments, R^{Z1A} is —N(R)C(O)OR. In some embodiments, R^{Z1A} is —N(R)C(O)R. In some embodiments, R^{Z1A} is —N(R)C(O)NR₂. In some embodiments, R^{Z1A} is —N(R)C(NR)NR₂. In some embodiments, R^{Z1A} is —N(R)S(O)₂NR₂. In some embodiments, R^{Z1A} is —N(R)S(O)₂R. In some embodiments, R^{Z1A} is —P(O)R₂. In some embodiments, R^{Z1A} is —P(O)(R)OR. In some embodiments, R^{Z1A} is —B(OR)₂. In some embodiments, R^{Z1A} is deuterium.

In some embodiments, R^{Z1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z1A} is halogen, —CN, or —NO₂. In some embodiments, R^{Z1A} is —OR, —SR, or —NR₂. In some embodiments, R^{Z1A} is —S(O)₂R, —S(O)₂NR₂, —S(O)₂F, —S(O)R, —S(O)NR₂, or —S(O)(NR)R. In some embodiments, R^{Z1A} is —C(O)R, —C(O)OR, —C(O)NR₂, or —C(O)N(R)OR. In some embodiments, R^{Z1A} is —OC(O)R or —OC(O)NR₂. In some embodiments, R^{Z1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{Z1A} is —P(O)R₂ or —P(O)(R)OR.

In some embodiments, R^{Z1A} is —OR, —OC(O)R, or —OC(O)NR₂. In some embodiments, R^{Z1A} is —SR, —S(O)₂R, —S(O)₂NR₂, —S(O)₂F, —S(O)R, —S(O)NR₂, or —S(O)(NR)R. In some embodiments, R^{Z1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{Z1A} is —S(O)₂R, —S(O)₂NR₂, or —S(O)₂F. In some embodiments, R^{Z1A} is —S(O)R, —S(O)NR₂, or —S(O)(NR)R. In some embodiments, R^{Z1A} is —SR, —S(O)₂R, or —S(O)R. In some embodiments, R^{Z1A} is —S(O)₂NR₂, —S(O)NR₂, or —S(O)(NR)R. In some embodiments, R^{Z1A} is —S(O)₂NR₂ or —S(O)NR₂. In some embodiments, R^{Z1A} is —SR, —S(O)₂R, —S(O)₂NR₂, or —S(O)R.

In some embodiments, R^{Z1A} is —N(R)C(O)OR, —N(R)C(O)R, or —N(R)C(O)NR₂. In some embodiments, R^{Z1A} is —N(R)S(O)₂NR₂ or —N(R)S(O)₂R. In some embodiments, R^{Z}A is —N(R)C(O)OR or —N(R)C(O)R. In some embodiments, R^{Z1A} is —N(R)C(O)NR₂ or —N(R)S(O)₂NR₂. In some embodiments, R^{Z1A} is —N(R)C(O)OR, —N(R)C(O)R, or —N(R)S(O)₂R.

In some embodiments, R^{Z1A} is —NR₂, —N(R)C(O)OR, —N(R)C(O)R, or —N(R)C(O)NR₂. In some embodiments, R^{Z1A} is —NR₂, —N(R)C(O)OR, or —N(R)C(O)R. In some embodiments, R^{Z1A} is —NR₂, —N(R)C(O)OR, —N(R)C(O)R, or —N(R)S(O)₂R.

In some embodiments, R^{Z1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is a C₁₋₆ aliphatic chain substituted by s⁵ instances of R^{Z1C}. In some embodiments, R^{Z1A} is phenyl substituted by s⁵ instances of R^{Z1C}. In some embodiments, R^{Z1A} is naphthyl substituted by s⁵ instances of R^{Z1C}. In some embodiments, R^{Z1A} is a 5-6 membered monocyclic heteroaryl ring having 1-4 heteroatoms independently selected from nitrogen, oxygen, and sulfur; wherein said ring is substituted by s⁵ instances of R^{Z1C}. In some embodiments, R^{Z1A} is an 8-10 membered bicyclic heteroaryl ring having 1-4 heteroatoms independently selected from nitrogen, oxygen, and sulfur; wherein said ring is substituted by s⁵ instances of R^{Z1C}. In some embodiments, R^{Z1A} is a 3-7 membered saturated or partially unsaturated monocyclic carbocyclic ring substituted by s⁵ instances of R^{Z1C}. In some embodiments, R^{Z1A} is a 5-12 membered saturated or partially unsaturated bicyclic carbocyclic ring substituted by s⁵ instances of R^{Z1C}. In some embodiments, R^{Z1A} is a 3-7 membered saturated or partially unsaturated monocyclic heterocyclic ring having 1-2 heteroatoms independently selected from nitrogen, oxygen, and sulfur; wherein said ring is substituted by s⁵ instances of R^{Z1C}. In some embodiments, R^{Z1A} is a 7-12 membered saturated or partially unsaturated bicyclic heterocyclic ring having 1-4 heteroatoms independently selected from nitrogen, oxygen, and sulfur; wherein said ring is substituted by s⁵ instances of R^{Z1C}.

In some embodiments, R^{Z1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁵ instances of R^{Z1C}. In some embodiments, R^{Z1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by s⁵ instances of R^{Z1C}. In some embodiments, R^{Z1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by ss instances of R^{Z1C}. In some embodiments, R^{Z1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl or naphthyl; each of which is substituted by s⁵ instances of R^{Z1C}. In some embodiments, R^{Z1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁵ instances of R^{Z1C}. In some embodiments, R^{Z1A} is a 3-7 membered saturated or partially unsaturated monocyclic carbocyclic ring or a 5-12 membered saturated or partially unsaturated bicyclic carbocyclic ring; each of which is substituted by s⁵ instances of R^{Z1C}. In some embodiments, R^{Z1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl or a 5-6 membered monocyclic heteroaryl ring having 1-4 heteroatoms independently selected from nitrogen, oxygen, and sulfur; each of which is substituted by s⁵ instances of R^{Z1C}. In some embodiments, R^{Z1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s⁵ instances of R^{Z1C}. In some embodiments, R^{Z1A} is naphthyl or an 8-10 membered bicyclic heteroaryl ring having 1-4 heteroatoms independently selected from nitrogen, oxygen, and sulfur; each of which is substituted by s⁵ instances of R^{Z1C}. In some embodiments, R^{Z1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is phenyl or a 3-7 membered saturated or partially unsaturated monocyclic carbocyclic ring; each of which is substituted by s⁵ instances of R^{Z1C} In some embodiments, R^{Z1A} is naphthyl or a 5-12 membered saturated or partially unsaturated bicyclic carbocyclic ring; each of which is substituted by s⁵ instances of R^{Z1C}. In some embodiments, R^{Z1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁵ instances of R^{Z1C}. In some embodiments, R^{Z1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}.

In some embodiments, R^{Z1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁵ instances of R^{Z1C}. In some embodiments, R^{Z1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s⁵ instances of R^{Z1C}. In some embodiments, R^{Z1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁵ instances of R^{Z1C}.

In some embodiments, R^{Z1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s⁵ instances of R^{Z1C}. In some embodiments, R^{Z1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s⁵ instances of R^{Z1C}. In some embodiments, R^{Z1A} is a C₁₋₆ aliphatic chain, phenyl, or a 3-7 membered saturated or partially unsaturated monocyclic carbocyclic ring; each of which is substituted by s⁵ instances of R^{Z1C}.

In some embodiments, R^{Z1A} is selected from the groups depicted in the compounds in Table 1.

As defined generally above, R^{Z2A} is R^{C} or R^{D} substituted by s⁶ instances of R^{Z2C}. In some embodiments, R^{Z2A} is R^{C}. In some embodiments, R^{Z2A} is R^{D} substituted by s⁶ instances of R^{Z2C}.

In some embodiments, R^{Z2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or R^{D} selected from a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s⁶ instances of R^{Z2C}.

In some embodiments, R^{Z2A} is halogen, —CN, —OR, —NR₂, —C(O)R, —C(O)OR, —C(O)NR₂, —N(R)C(O)R, or R^{D} selected from a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s⁶ instances of R^{Z2C}.

In some embodiments, R^{Z2A} is halogen, —CN, —OR, —NR₂, —C(O)R, —C(O)OR, —C(O)NR₂, —N(R)C(O)R, or R^{D} selected from a C₁₋₆ aliphatic chain; a 5-6 membered monocyclic heteroaryl ring having 1-4 heteroatoms independently selected from nitrogen, oxygen, and sulfur; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s⁶ instances of R^{Z2C} In some embodiments, R^{Z2A} is halogen or R^{D} selected from a C₁₋₆ aliphatic chain; a 5-6 membered monocyclic heteroaryl ring having 1-4 heteroatoms independently selected from nitrogen, oxygen, and sulfur; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s⁶ instances of R^{Z2C}.

In some embodiments, R^{Z2A} is halogen or R^{D} selected from a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁶ instances of R^{Z2C} In some embodiments, R^{Z2A} is halogen or R^{D} selected from a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁶ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{Z2A} is halogen or R^{D} selected from a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁶ instances of a group independently selected from halogen, —CN, —OH, —O-(optionally substituted C₁₋₃ aliphatic), and an optionally substituted C₁₋₃ aliphatic. In some embodiments, R^{Z2A} is halogen or R^{D} selected from a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁶ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z2A} is halogen or R^{D} selected from a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁶ instances of a group independently selected from fluorine, chlorine, —OH, —OCH₃, —OCF₃, —CH₃, —CHF₂, and —CF₃.

In some embodiments, R^{Z2A} is halogen or R^{D} selected from a C₁₋₄ aliphatic chain and a 3-5 membered saturated or partially unsaturated monocyclic heterocyclic ring having 1-2 heteroatoms independently selected from nitrogen, oxygen, and sulfur; wherein said R^{D} is substituted by s⁶ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z2A} is halogen or R^{D} selected from a C₁₋₄ aliphatic chain and a 3-5 membered saturated or partially unsaturated monocyclic heterocyclic ring having 1-2 heteroatoms independently selected from nitrogen and oxygen; wherein said R^{D} is substituted by s⁶ instances of a group independently selected from fluorine, chlorine, —OH, —OCH₃, —OCF₃, —CH₃, —CHF₂, and —CF₃.

In some embodiments, R^{Z2A} is halogen or a C₁₋₄ aliphatic chain substituted by —OH and 0-3 fluorine.

In some embodiments, R^{Z2A} is halogen, —CN, —OR, —NR₂, —C(O)R, —C(O)OR, —C(O)NR₂, or —N(R)C(O)R. In some embodiments, R^{Z2A} is halogen.

In some embodiments, R^{Z2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; wherein R^{Z2A} is substituted by s⁶ instances of R^{Z2C}. In some embodiments, R^{Z2A} is a C₁₋₆ aliphatic chain; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; wherein R^{Z2A} is substituted by s⁶ instances of R^{Z2C}.

In some embodiments, R^{Z2A} is a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of R^{Z2C}. In some embodiments, R^{Z2A} is a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{Z2A} is a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —CN, —OH, —O-(optionally substituted C₁₋₃ aliphatic), and an optionally substituted C₁₋₃ aliphatic. In some embodiments, R^{Z2A} is a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z2A} is a C₁₋₄ aliphatic chain; a 5-6 membered monocyclic heteroaryl ring having 1-4 heteroatoms independently selected from nitrogen, oxygen, and sulfur; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from fluorine, chlorine, —OH, —OCH₃, —OCF₃, —CH₃, —CHF₂, and —CF₃.

In some embodiments, R^{Z2A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of R^{Z2C}. In some embodiments, R^{Z2A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —CN, —OH, —O-(optionally substituted C₁₋₃ aliphatic), and an optionally substituted C₁₋₃ aliphatic. In some embodiments, R^{Z2A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z2A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from fluorine, chlorine, —OH, —OCH₃, —OCF₃, —CH₃, —CHF₂, and —CF₃.

In some embodiments, R^{Z2A} is a C₁₋₄ aliphatic chain or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z2A} is a C₁₋₄ aliphatic chain or a 3-5 membered saturated or partially unsaturated monocyclic heterocyclic ring having 1-2 heteroatoms independently selected from nitrogen and oxygen; wherein R^{Z2A} is substituted by s⁶ instances of a group independently selected from fluorine, chlorine, —OH, —OCH₃, —OCF₃, —CH₃, —CHF₂, and —CF₃. In some embodiments, R^{Z2A} is a aliphatic chain substituted by —OH and 0-3 fluorine.

In some embodiments, R^{Z2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{Z2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z2A} is oxo. In some embodiments, R^{Z2A} is halogen. In some embodiments, R^{Z2A} is —CN. In some embodiments, R^{Z2A} is —NO₂. In some embodiments, R^{Z2A} is —OR. In some embodiments, R^{Z2A} is —SR. In some embodiments, R^{Z2A} is —NR₂. In some embodiments, R^{Z2A} is —S(O)₂R. In some embodiments, R^{Z2A} is —S(O)₂NR₂. In some embodiments, R^{Z2A} is —S(O)₂F. In some embodiments, R^{Z2A} is —S(O)R. In some embodiments, R^{Z2A} is —S(O)NR₂. In some embodiments, R^{Z2A} is —S(O)(NR)R. In some embodiments, R^{Z2A} is —C(O)R. In some embodiments, R^{Z2A} is —C(O)OR. In some embodiments, R^{Z2A} is —C(O)NR₂. In some embodiments, R^{Z2A} is —C(O)N(R)OR. In some embodiments, R^{Z2A} is —OC(O)R. In some embodiments, R^{Z2A} is —OC(O)NR₂. In some embodiments, R^{Z2A} is —N(R)C(O)OR. In some embodiments, R^{Z2A} is —N(R)C(O)R. In some embodiments, R^{Z2A} is —N(R)C(O)NR₂. In some embodiments, R^{Z2A} is —N(R)C(NR)NR₂. In some embodiments, R^{Z2A} is —N(R)S(O)₂NR₂. In some embodiments, R^{Z2A} is —N(R)S(O)₂R. In some embodiments, R^{Z2A} is —P(O)R₂. In some embodiments, R^{Z2A} is —P(O)(R)OR. In some embodiments, R^{Z2A} is —B(OR)₂. In some embodiments, R^{Z2A} is deuterium.

In some embodiments, R^{Z2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z2A} is halogen, —CN, or —NO₂. In some embodiments, R^{Z2A} is —OR, —SR, or —NR₂. In some embodiments, R^{Z2A} is —S(O)₂R, —S(O)₂NR₂, —S(O)₂F, —S(O)R, —S(O)NR₂, or —S(O)(NR)R. In some embodiments, R^{Z2A} is —C(O)R, —C(O)OR, —C(O)NR₂, or —C(O)N(R)OR. In some embodiments, R^{Z2A} is —OC(O)R or —OC(O)NR₂. In some embodiments, R^{Z2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{Z2A} is —P(O)R₂ or —P(O)(R)OR.

In some embodiments, R^{Z2A} is —OR, —OC(O)R, or —OC(O)NR₂. In some embodiments, R^{Z2A} is —SR, —S(O)₂R, —S(O)₂NR₂, —S(O)₂F, —S(O)R, —S(O)NR₂, or —S(O)(NR)R. In some embodiments, R^{Z2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{Z2A} is —S(O)₂R, —S(O)₂NR₂, or —S(O)₂F. In some embodiments, R^{Z2A} is —S(O)R, —S(O)NR₂, or —S(O)(NR)R. In some embodiments, R^{Z2A} is —SR, —S(O)₂R, or —S(O)R. In some embodiments, R^{Z2A} is —S(O)₂NR₂, —S(O)NR₂, or —S(O)(NR)R. In some embodiments, R^{Z2A} is —S(O)₂NR₂ or —S(O)NR₂. In some embodiments, R^{Z2A} is —SR, —S(O)₂R, —S(O)₂NR₂, or —S(O)R.

In some embodiments, R^{Z2A} is —N(R)C(O)OR, —N(R)C(O)R, or —N(R)C(O)NR₂. In some embodiments, R^{Z2A} is —N(R)S(O)₂NR₂ or —N(R)S(O)₂R. In some embodiments, R^{Z2A} is —N(R)C(O)OR or —N(R)C(O)R. In some embodiments, R^{Z2A} is —N(R)C(O)NR₂ or —N(R)S(O)₂NR₂. In some embodiments, R^{Z2A} is —N(R)C(O)OR, —N(R)C(O)R, or —N(R)S(O)₂R.

In some embodiments, R^{Z2A} is —NR₂, —N(R)C(O)OR, —N(R)C(O)R, or —N(R)C(O)NR₂. In some embodiments, R^{Z2A} is —NR₂, —N(R)C(O)OR, or —N(R)C(O)R. In some embodiments, R^{Z2A} is —NR₂, —N(R)C(O)OR, —N(R)C(O)R, or —N(R)S(O)₂R.

In some embodiments, R^{Z2A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is a C₁₋₆ aliphatic chain substituted by s⁶ instances of R^{Z2C}. In some embodiments, R^{Z2A} is phenyl substituted by s⁶ instances of R^{Z2C}. In some embodiments, R^{Z2A} is naphthyl substituted by s⁶ instances of R^{Z2C}. In some embodiments, R^{Z2A} is a 5-6 membered monocyclic heteroaryl ring having 1-4 heteroatoms independently selected from nitrogen, oxygen, and sulfur; wherein said ring is substituted by s⁶ instances of R^{Z2C}. In some embodiments, R^{Z2A} is an 8-10 membered bicyclic heteroaryl ring having 1-4 heteroatoms independently selected from nitrogen, oxygen, and sulfur; wherein said ring is substituted by s⁶ instances of R^{Z2C}. In some embodiments, R^{Z2A} is a 3-7 membered saturated or partially unsaturated monocyclic carbocyclic ring substituted by s⁶ instances of R^{Z2C}. In some embodiments, R^{Z2A} is a 5-12 membered saturated or partially unsaturated bicyclic carbocyclic ring substituted by s⁶ instances of R^{Z2C}. In some embodiments, R^{Z2A} is a 3-7 membered saturated or partially unsaturated monocyclic heterocyclic ring having 1-2 heteroatoms independently selected from nitrogen, oxygen, and sulfur; wherein said ring is substituted by s⁶ instances of R^{Z2C}. In some embodiments, R^{Z2A} is a 7-12 membered saturated or partially unsaturated bicyclic heterocyclic ring having 1-4 heteroatoms independently selected from nitrogen, oxygen, and sulfur; wherein said ring is substituted by s⁶ instances of R^{Z2C}.

In some embodiments, R^{Z2A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁶ instances of R^{Z2C}. In some embodiments, R^{Z2A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl; naphthyl; a 3-7 membered saturated or partially unsaturated monocyclic carbocyclic ring; or a 5-12 membered saturated or partially unsaturated bicyclic carbocyclic ring; each of which is substituted by s⁶ instances of R^{Z2C}. In some embodiments, R^{Z2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}. In some embodiments, R^{Z2A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl or naphthyl; each of which is substituted by s⁶ instances of R^{Z2C}. In some embodiments, R^{Z2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁶ instances of R^{Z2C}. In some embodiments, R^{Z2A} is a 3-7 membered saturated or partially unsaturated monocyclic carbocyclic ring or a 5-12 membered saturated or partially unsaturated bicyclic carbocyclic ring; each of which is substituted by s⁶ instances of R^{Z2C}. In some embodiments, R^{Z2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl or a 5-6 membered monocyclic heteroaryl ring having 1-4 heteroatoms independently selected from nitrogen, oxygen, and sulfur; each of which is substituted by s⁶ instances of R^{Z2C}. In some embodiments, R^{Z2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}. In some embodiments, R^{Z2A} is naphthyl or an 8-10 membered bicyclic heteroaryl ring having 1-4 heteroatoms independently selected from nitrogen, oxygen, and sulfur; each of which is substituted by s⁶ instances of R^{Z2C}. In some embodiments, R^{Z2A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is phenyl or a 3-7 membered saturated or partially unsaturated monocyclic carbocyclic ring; each of which is substituted by s⁶ instances of R^{Z2C} In some embodiments, R^{Z2A} is naphthyl or a 5-12 membered saturated or partially unsaturated bicyclic carbocyclic ring; each of which is substituted by s⁶ instances of R^{Z2C}. In some embodiments, R^{Z2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}. In some embodiments, R^{Z2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}.

In some embodiments, R^{Z2A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁶ instances of R^{Z2C}. In some embodiments, R^{Z2A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s⁶ instances of R^{Z2C}. In some embodiments, R^{Z2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}.

In some embodiments, R^{Z2A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s⁶ instances of R^{Z2C}. In some embodiments, R^{Z2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}. In some embodiments, R^{Z2A} is a C₁₋₆ aliphatic chain, phenyl, or a 3-7 membered saturated or partially unsaturated monocyclic carbocyclic ring; each of which is substituted by s⁶ instances of R^{Z2C}.

In some embodiments, R^{Z2A} is an 8-10 membered bicyclic heteroaryl ring having 1, 2, or 3 heteroatoms independently selected from nitrogen, oxygen, and sulfur; wherein said ring is substituted by s⁶ instances of R^{Z2C}. In some embodiments, R^{Z2A} is an 8-10 membered bicyclic heteroaryl ring having 2 or 3 heteroatoms independently selected from nitrogen, oxygen, and sulfur; wherein said ring is substituted by s⁶ instances of R^{Z2C}. In some embodiments, R^{Z2A} is an 8-10 membered bicyclic heteroaryl ring having 1, 2, or 3 nitrogen atoms; wherein said ring is substituted by s⁶ instances of R^{Z2C}. In some embodiments, R^{Z2A} is an 8-10 membered bicyclic heteroaryl ring having 2 or 3 nitrogen atoms; wherein said ring is substituted by s⁶ instances of R^{Z2C}.

In certain embodiments, R^{Z2A} is imidazo[1,2-a]pyrazinyl, [1,2,4]triazolo[1,5-a]pyridinyl, or pyrazolo[1,5-a]pyrimidinyl; each of which is substituted by s⁶ instances of R^{Z2C}. In certain embodiments, R^{Z2A} is imidazo[1,2-a]pyrazinyl substituted by s⁶ instances of R^{Z2C}. In certain embodiments, R^{Z2A} is [1,2,4]triazolo[1,5-a]pyridinyl substituted by s⁶ instances of R^{Z2C}. In certain embodiments, R^{Z2A} is pyrazolo[1,5-a]pyrimidinyl substituted by s⁶ instances of R^{Z2C}.

In certain embodiments, R^{Z2A} is imidazo[1,2-a]pyrazin-6-yl, [1,2,4]triazolo[1,5-a]pyridin-6-yl, or pyrazolo[1,5-a]pyrimidin-5-yl; each of which is substituted by s⁶ instances of R^{Z2C}. In certain embodiments, R^{Z2A} is imidazo[1,2-a]pyrazin-6-yl substituted by s⁶ instances of R^{Z2C}. In certain embodiments, R^{Z2A} is [1,2,4]triazolo[1,5-a]pyridin-6-yl substituted by s⁶ instances of R^{Z2C}. In certain embodiments, R^{Z2A} is pyrazolo[1,5-a]pyrimidin-5-yl substituted by s⁶ instances of R^{Z2C}.

In certain embodiments, R^{Z2A} is imidazo[1,2-a]pyrazin-6-yl, [1,2,4]triazolo[1,5-a]pyridin-6-yl, or pyrazolo[1,5-a]pyrimidin-5-yl. In certain embodiments, R^{Z2A} is imidazo[1,2-a]pyrazin-6-yl. In certain embodiments, R^{Z2A} is [1,2,4]triazolo[1,5-a]pyridin-6-yl. In certain embodiments, R^{Z2A} is pyrazolo[1,5-a]pyrimidin-5-yl.

In some embodiments, R^{Z2A} is a 5-6 membered monocyclic heteroaryl ring having 1, 2, or 3 heteroatoms independently selected from nitrogen, oxygen, and sulfur; or a 5-7 membered saturated or partially unsaturated monocyclic heterocyclic ring having 1 or 2 heteroatoms independently selected from nitrogen, oxygen, and sulfur; each of which is substituted by s⁶ instances of R^{Z2C}.

In some embodiments, R^{Z2A} is a 5-6 membered monocyclic heteroaryl ring having 1, 2, or 3 heteroatoms independently selected from nitrogen, oxygen, and sulfur; wherein said ring is substituted by s⁶ instances of R^{Z2C}. In some embodiments, R^{Z2A} is a 5-membered monocyclic heteroaryl ring having 1, 2, or 3 heteroatoms independently selected from nitrogen, oxygen, and sulfur; wherein said ring is substituted by s⁶ instances of R^{Z2C}. In some embodiments, R^{Z2A} is a 5-membered monocyclic heteroaryl ring having 1 or 2 heteroatoms independently selected from nitrogen and oxygen; wherein said ring is substituted by s⁶ instances of R^{Z2C}. In some embodiments, R^{Z2A} is a 6-membered monocyclic heteroaryl ring having 1 or 2 nitrogen atoms; wherein said ring is substituted by s⁶ instances of R^{Z2C}.

In certain embodiments, R^{Z2A} is pyrazolyl or imidazolyl, each of which is substituted by s⁶ instances of R^{Z2C}. In certain embodiments, R^{Z2A} is pyrazolyl substituted by s⁶ instances of R^{Z2C}. In certain embodiments, R^{Z2A} is imidazolyl substituted by s⁶ instances of R^{Z2C}. In certain embodiments, R^{Z2A} is pyrazol-4-yl or imidazol-4-yl, each of which is substituted by s⁶ instances of R^{Z2C}. In certain embodiments, R^{Z2A} is pyrazol-4-yl substituted by s⁶ instances of R^{Z2C}. In certain embodiments, R^{Z2A} is imidazol-4-yl substituted by s⁶ instances of R^{Z2C}.

In some embodiments, R^{Z2A} is a 3-7 membered partially unsaturated monocyclic heterocyclic ring having 1 or 2 heteroatoms independently selected from nitrogen, oxygen, and sulfur; wherein said ring is substituted by s⁶ instances of R^{Z2C}. In some embodiments, R^{Z2A} is a 3-7 membered saturated monocyclic heterocyclic ring having 1 or 2 heteroatoms independently selected from nitrogen, oxygen, and sulfur; wherein said ring is substituted by s⁶ instances of R^{Z2C}.

In some embodiments, R^{Z2A} is a 5-7 membered saturated or partially unsaturated monocyclic heterocyclic ring having 1 or 2 heteroatoms independently selected from nitrogen, oxygen, and sulfur; wherein said ring is substituted by s⁶ instances of R^{Z2C}. In some embodiments, R^{Z2A} is a 5-7 membered saturated monocyclic heterocyclic ring having 1 or 2 heteroatoms independently selected from nitrogen, oxygen, and sulfur; wherein said ring is substituted by s⁶ instances of R^{Z2C}. In some embodiments, R^{Z2A} is a 5-7 membered partially unsaturated monocyclic heterocyclic ring having 1 or 2 heteroatoms independently selected from nitrogen, oxygen, and sulfur; wherein said ring is substituted by s⁶ instances of R^{Z2C}.

In some embodiments, R^{Z2A} is a 6-membered saturated or partially unsaturated monocyclic heterocyclic ring having 1 or 2 heteroatoms independently selected from nitrogen and oxygen; wherein said ring is substituted by s⁶ instances of R^{Z2C}. In some embodiments, R^{Z2A} is a 6-membered saturated monocyclic heterocyclic ring having 1 or 2 heteroatoms independently selected from nitrogen and oxygen; wherein said ring is substituted by s⁶ instances of R^{Z2C}. In some embodiments, R^{Z2A} is a 6-membered partially unsaturated monocyclic heterocyclic ring having 1 or 2 heteroatoms independently selected from nitrogen and oxygen; wherein said ring is substituted by s⁶ instances of R^{Z2C}. In some embodiments, R^{Z2A} is pyridin-2(1H)-onyl substituted by 0, 1, 2, or 3 instances of R^{Z2C}. In some embodiments, R^{Z2A} is pyridin-2(1H)-on-5-yl substituted by 0, 1, 2, or 3 instances of R^{Z2C}.

In some embodiments, R^{Z2A} is

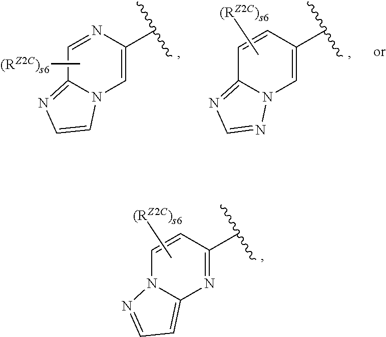

wherein R^{Z2C} and s⁶ are as defined in the embodiments and classes and subclasses herein. In some embodiments, R^{Z2A} is

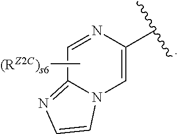

In some embodiments, R^{Z2A} is

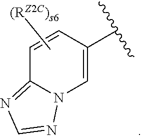

In some embodiments, R^{Z2A} is

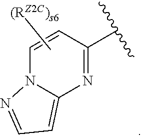

In some embodiments, R^{Z2A} is

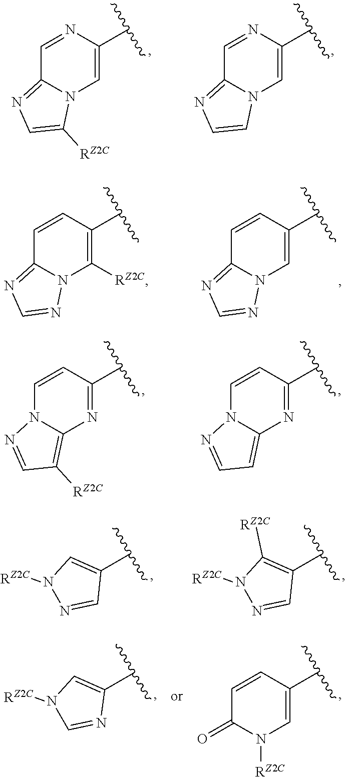

wherein R^{Z2C} is as defined in the embodiments and classes and subclasses herein. In some embodiments, R^{Z2A} is

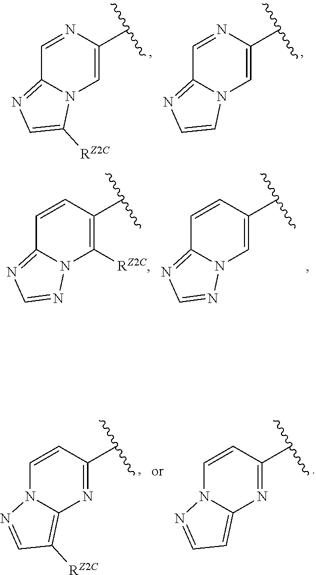

In some embodiments, R^{Z2A} is

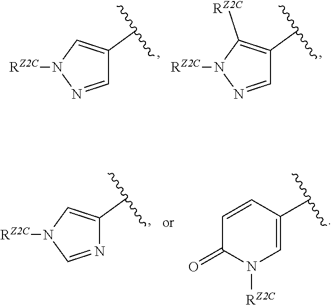

In some embodiments, R^{Z2A} is

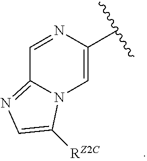

In some embodiments, R^{Z2A} is

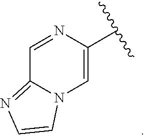 [SMILES: *c1cn2ccnc2cn1]

In some embodiments, R^{Z2A} is

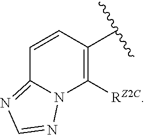

In some embodiments, R^{Z2A} is

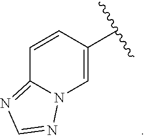 [SMILES: *c1ccc2ncnn2c1]

In some embodiments, R^{Z2A} is

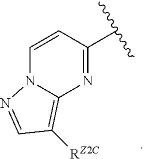

In some embodiments, R^{Z2A} is

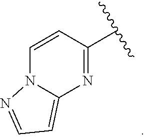 [SMILES: *c1ccn2nccc2n1]

In some embodiments, R^{Z2A} is

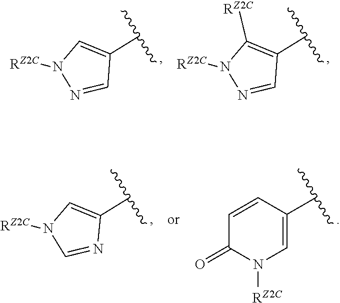

In some embodiments, R^{Z2A} is

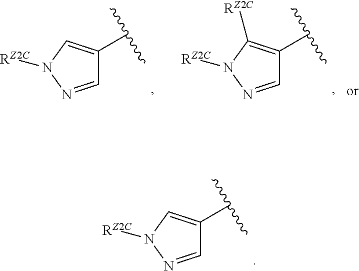

In some embodiments, R^{Z2A} is

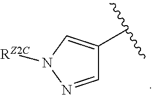

In some embodiments, R^{Z2A} is

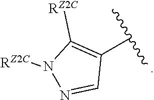

In some embodiments, R^{Z2A} is

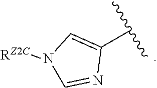

In some embodiments, R^{Z2A} is

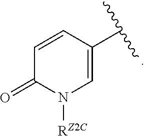

In some embodiments, R^{Z2A} is selected from the groups depicted in the compounds in Table 1.

As defined generally above, R^{Z3A} is R^{C} or R^{D} substituted by s instances of R^{Z3C}. In some embodiments, R^{Z3A} is R^{C}. In some embodiments, R^{Z3A} is R^{D} substituted by s⁷ instances of R^{Z3C}.

In some embodiments, R^{Z3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or R^{D} selected from a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s⁷ instances of R^{Z3C}.

In some embodiments, R^{Z3A} is halogen, —CN, —OR, —NR₂, —C(O)R, —C(O)OR, —C(O)NR₂, —N(R)C(O)R, or R^{D} selected from a C₁₋₆ aliphatic chain; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; wherein said R^{D} is substituted by s instances of R^{Z3C}. In some embodiments, R^{Z3A} is halogen or R^{D} selected from a C₁₋₄ aliphatic chain; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁷ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{Z3A} is halogen or R^{D} selected from a C₁₋₄ aliphatic chain; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁷ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z3A} is halogen or R^{D} selected from a C₁₋₄ aliphatic chain; a 3-5 membered saturated or partially unsaturated monocyclic carbocyclic ring; and a 3-5 membered saturated or partially unsaturated monocyclic heterocyclic ring having 1-2 heteroatoms independently selected from nitrogen, oxygen, and sulfur; wherein said R^{D} is substituted by s⁷ instances of a group independently selected from fluorine, chlorine, —OH, —OCH₃, —OCF₃, —CH₃, —CHF₂, and —CF₃.

In some embodiments, R^{Z3A} is halogen, —CN, —OR, —NR₂, —C(O)R, —C(O)OR, —C(O)NR₂, or —N(R)C(O)R. In some embodiments, R^{Z3A} is halogen.

In some embodiments, R^{Z3A} is a C₁₋₆ aliphatic chain; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; wherein R^{Z3A} is substituted by s⁷ instances of R^{Z3C}. In some embodiments, R^{Z3A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z3A} is substituted by s⁷ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{Z3A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z3A} is substituted by s⁷ instances of a group independently selected from halogen, —OH, —O—(C₁₋₃ aliphatic), and C₁₋₃ aliphatic, wherein each C₁₋₃ aliphatic is optionally substituted with 1-3 halogen. In some embodiments, R^{Z3A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z3A} is substituted by s⁷ instances of a group independently selected from fluorine, chlorine, —OH, —OCH₃, —OCF₃, —CH₃, —CHF₂, and —CF₃.

In some embodiments, R^{Z3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{Z3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z3A} is oxo. In some embodiments, R^{Z3A} is halogen. In some embodiments, R^{Z3A} is —CN. In some embodiments, R^{Z3A} is —NO₂. In some embodiments, R^{Z3A} is —OR. In some embodiments, R^{Z3A} is —SR. In some embodiments, R^{Z3A} is —NR₂. In some embodiments, R^{Z3A} is —S(O)₂R. In some embodiments, R^{Z3A} is —S(O)₂NR₂. In some embodiments, R^{Z3A} is —S(O)₂F. In some embodiments, R^{Z3A} is —S(O)R. In some embodiments, R^{Z3A} is —S(O)NR₂. In some embodiments, R^{Z3A} is —S(O)(NR)R. In some embodiments, R^{Z3A} is —C(O)R. In some embodiments, R^{Z3A} is —C(O)OR. In some embodiments, R^{Z3A} is —C(O)NR₂. In some embodiments, R^{Z3A} is —C(O)N(R)OR. In some embodiments, R^{Z3A} is —OC(O)R. In some embodiments, R^{Z3A} is —OC(O)NR₂. In some embodiments, R^{Z3A} is —N(R)C(O)OR. In some embodiments, R^{Z3A} is —N(R)C(O)R. In some embodiments, R^{Z3A} is —N(R)C(O)NR₂. In some embodiments, R^{Z3A} is —N(R)C(NR)NR₂. In some embodiments, R^{Z3A} is —N(R)S(O)₂NR₂. In some embodiments, R^{Z3A} is —N(R)S(O)₂R. In some embodiments, R^{Z3A} is —P(O)R₂. In some embodiments, R^{Z3A} is —P(O)(R)OR. In some embodiments, R^{Z3A} is —B(OR)₂. In some embodiments, R^{Z3A} is deuterium.

In some embodiments, R^{Z3A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Z3A} is halogen, —CN, or —NO₂. In some embodiments, R^{Z3A} is —OR, —SR, or —NR₂. In some embodiments, R^{Z3A} is —S(O)₂R, —S(O)₂NR₂, —S(O)₂F, —S(O)R, —S(O)NR₂, or —S(O)(NR)R. In some embodiments, R^{Z3A} is —C(O)R, —C(O)OR, —C(O)NR₂, or —C(O)N(R)OR. In some embodiments, R^{Z3A} is —OC(O)R or —OC(O)NR₂. In some embodiments, R^{Z3A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{Z3A} is —P(O)R₂ or —P(O)(R)OR.

In some embodiments, R^{Z3A} is —OR, —OC(O)R, or —OC(O)NR₂. In some embodiments, R^{Z3A} is —SR, —S(O)₂R, —S(O)₂NR₂, —S(O)₂F, —S(O)R, —S(O)NR₂, or —S(O)(NR)R. In some embodiments, R^{Z3A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{Z3A} is —S(O)₂R, —S(O)₂NR₂, or —S(O)₂F. In some embodiments, R^{Z3A} is —S(O)R, —S(O)NR₂, or —S(O)(NR)R. In some embodiments, R^{Z3A} is —SR, —S(O)₂R, or —S(O)R. In some embodiments, R^{Z3A} is —S(O)₂NR₂, —S(O)NR₂, or —S(O)(NR)R. In some embodiments, R^{Z3A} is —S(O)₂NR₂ or —S(O)NR₂. In some embodiments, R^{Z3A} is —SR, —S(O)₂R, —S(O)₂NR₂, or —S(O)R.

In some embodiments, R^{Z3A} is —N(R)C(O)OR, —N(R)C(O)R, or —N(R)C(O)NR₂. In some embodiments, R^{Z3A} is —N(R)S(O)₂NR₂ or —N(R)S(O)₂R. In some embodiments, R^{Z3A} is —N(R)C(O)OR or —N(R)C(O)R. In some embodiments, R^{Z3A} is —N(R)C(O)NR₂ or —N(R)S(O)₂NR₂. In some embodiments, R^{Z3A} is —N(R)C(O)OR, —N(R)C(O)R, or —N(R)S(O)₂R.

In some embodiments, R^{Z3A} is —NR₂, —N(R)C(O)OR, —N(R)C(O)R, or —N(R)C(O)NR₂. In some embodiments, R^{Z3A} is —NR₂, —N(R)C(O)OR, or —N(R)C(O)R. In some embodiments, R^{Z3A} is —NR₂, —N(R)C(O)OR, —N(R)C(O)R, or —N(R)S(O)₂R.

In some embodiments, R^{Z3A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is a C₁₋₆ aliphatic chain substituted by s⁷ instances of R^{Z3C}. In some embodiments, R^{Z3A} is phenyl substituted by s⁷ instances of R^{Z3C}. In some embodiments, R^{Z3A} is naphthyl substituted by s⁷ instances of R^{Z3C}. In some embodiments, R^{Z3A} is a 5-6 membered monocyclic heteroaryl ring having 1-4 heteroatoms independently selected from nitrogen, oxygen, and sulfur; wherein said ring is substituted by s⁷ instances of R^{Z3C}. In some embodiments, R^{Z3A} is an 8-10 membered bicyclic heteroaryl ring having 1-4 heteroatoms independently selected from nitrogen, oxygen, and sulfur; wherein said ring is substituted by s⁷ instances of R^{Z3C}. In some embodiments, R^{Z3A} is a 3-7 membered saturated or partially unsaturated monocyclic carbocyclic ring substituted by s⁷ instances of R^{Z3C}. In some embodiments, R^{Z3A} is a 5-12 membered saturated or partially unsaturated bicyclic carbocyclic ring substituted by s⁷ instances of R^{Z3C}. In some embodiments, R^{Z3A} is a 3-7 membered saturated or partially unsaturated monocyclic heterocyclic ring having 1-2 heteroatoms independently selected from nitrogen, oxygen, and sulfur; wherein said ring is substituted by s⁷ instances of R^{Z3C}. In some embodiments, R^{Z3A} is a 7-12 membered saturated or partially unsaturated bicyclic heterocyclic ring having 1-4 heteroatoms independently selected from nitrogen, oxygen, and sulfur; wherein said ring is substituted by s⁷ instances of R^{Z3C}.

In some embodiments, R^{Z3A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁷ instances of R^{Z3C}. In some embodiments, R^{Z3A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl; naphthyl; a 3-7 membered saturated or partially unsaturated monocyclic carbocyclic ring; or a 5-12 membered saturated or partially unsaturated bicyclic carbocyclic ring; each of which is substituted by s⁷ instances of R^{Z3C}. In some embodiments, R^{Z3A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁷ instances of R^{Z3C}. In some embodiments, R^{Z3A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl or naphthyl; each of which is substituted by s⁷ instances of R^{Z3C}. In some embodiments, R^{Z3A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁷ instances of R^{Z3C}. In some embodiments, R^{Z3A} is a 3-7 membered saturated or partially unsaturated monocyclic carbocyclic ring or a 5-12 membered saturated or partially unsaturated bicyclic carbocyclic ring; each of which is substituted by s⁷ instances of R^{Z3C}. In some embodiments, R^{Z3A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl or a 5-6 membered monocyclic heteroaryl ring having 1-4 heteroatoms independently selected from nitrogen, oxygen, and sulfur; each of which is substituted by s⁷ instances of R^{Z3C}. In some embodiments, R^{Z3A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s⁷ instances of R^{Z3C}. In some embodiments, R^{Z3A} is naphthyl or an 8-10 membered bicyclic heteroaryl ring having 1-4 heteroatoms independently selected from nitrogen, oxygen, and sulfur; each of which is substituted by s⁷ instances of R^{Z3C}. In some embodiments, R^{Z3A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is phenyl or a 3-7 membered saturated or partially unsaturated monocyclic carbocyclic ring; each of which is substituted by s⁷ instances of R^{Z3C} In some embodiments, R^{Z3A} is naphthyl or a 5-12 membered saturated or partially unsaturated bicyclic carbocyclic ring; each of which is substituted by s⁷ instances of R^{Z3C}. In some embodiments, R^{Z3A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁷ instances of R^{Z3C}. In some embodiments, R^{Z3A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}.

In some embodiments, R^{Z3A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{Z3C}. In some embodiments, R^{Z3A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s⁷ instances of R^{Z3C}. In some embodiments, R^{Z3A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁷ instances of R^{Z3C}.

In some embodiments, R^{Z3A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s⁷ instances of R^{Z3C}. In some embodiments, R^{Z3A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s⁷ instances of R^{Z3C}. In some embodiments, R^{Z3A} is a C₁₋₆ aliphatic chain, phenyl, or a 3-7 membered saturated or partially unsaturated monocyclic carbocyclic ring; each of which is substituted by s⁷ instances of R^{Z3C}.

In some embodiments, R^{Z3A} is selected from the groups depicted in the compounds in Table 1.

As defined generally above, R_{L1} is R^{C} or R^{D} substituted by s⁸ instances of R^{L1C}. In some embodiments, R^{L1} is R^{C}. In some embodiments, R^{L1} is R^{D} substituted by s⁸ instances of R^{L1C}.

In some embodiments, R^{L1} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L1} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L1} is oxo. In some embodiments, R^{L1} is halogen. In some embodiments, R^{L1} is —CN. In some embodiments, R^{L1} is —NO₂. In some embodiments, R^{L1} is —OR. In some embodiments, R^{L1} is —SR. In some embodiments, R^{L1} is —NR₂. In some embodiments, R^{L1} is —S(O)₂R. In some embodiments, R^{L1} is —S(O)₂NR₂. In some embodiments, R^{L1} is —S(O)₂F. In some embodiments, R^{L1} is —S(O)R. In some embodiments, R^{L1} is —S(O)NR₂. In some embodiments, R^{L1} is —S(O)(NR)R. In some embodiments, R^{L1} is —C(O)R. In some embodiments, R^{L1} is —C(O)OR. In some embodiments, R^{L1} is —C(O)NR₂. In some embodiments, R^{L1} is —C(O)N(R)OR. In some embodiments, R^{L1} is —OC(O)R. In some embodiments, R^{L1} is —OC(O)NR₂. In some embodiments, R^{L1} is —N(R)C(O)OR. In some embodiments, R^{L1} is —N(R)C(O)R. In some embodiments, R^{L1} is —N(R)C(O)NR₂. In some embodiments, R^{L1} is —N(R)C(NR)NR₂. In some embodiments, R^{L1} is —N(R)S(O)₂NR₂. In some embodiments, R^{L1} is —N(R)S(O)₂R. In some embodiments, R^{L1} is —P(O)R₂. In some embodiments, R^{L1} is —P(O)(R)OR. In some embodiments, R^{L1} is —B(OR)₂. In some embodiments, R^{L1} is deuterium.

In some embodiments, R^{L1} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L1} is halogen, —CN, or —NO₂. In some embodiments, R^{L1} is —OR, —SR, or —NR₂. In some embodiments, R^{L1} is —S(O)₂R, —S(O)₂NR₂, —S(O)₂F, —S(O)R, —S(O)NR₂, or —S(O)(NR)R. In some embodiments, R^{L1} is —C(O)R, —C(O)OR, —C(O)NR₂, or —C(O)N(R)OR. In some embodiments, R^{L1} is —OC(O)R or —OC(O)NR₂. In some embodiments, R^{L1} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L1} is —P(O)R₂ or —P(O)(R)OR.

In some embodiments, R^{L1} is —OR, —OC(O)R, or —OC(O)NR₂. In some embodiments, R^{L1} is —SR, —S(O)₂R, —S(O)₂NR₂, —S(O)₂F, —S(O)R, —S(O)NR₂, or —S(O)(NR)R. In some embodiments, R^{L1} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L1} is —S(O)₂R, —S(O)₂NR₂, or —S(O)₂F. In some embodiments, R^{L1} is —S(O)R, —S(O)NR₂, or —S(O)(NR)R. In some embodiments, R^{L1} is —SR, —S(O)₂R, or —S(O)R. In some embodiments, R^{L1} is —S(O)₂NR₂, —S(O)NR₂, or —S(O)(NR)R. In some embodiments, R^{L1} is —S(O)₂NR₂ or —S(O)NR₂. In some embodiments, R^{L1} is —SR, —S(O)₂R, —S(O)₂NR₂, or —S(O)R.

In some embodiments, R^{L1} is —N(R)C(O)OR, —N(R)C(O)R, or —N(R)C(O)NR₂. In some embodiments, R^{L1} is —N(R)S(O)₂NR₂ or —N(R)S(O)₂R. In some embodiments, R^{L1} is —N(R)C(O)OR or —N(R)C(O)R. In some embodiments, R^{L1} is —N(R)C(O)NR₂ or —N(R)S(O)₂NR₂. In some embodiments, R^{L1} is —N(R)C(O)OR, —N(R)C(O)R, or —N(R)S(O)₂R.

In some embodiments, R^{L1} is —NR₂, —N(R)C(O)OR, —N(R)C(O)R, or —N(R)C(O)NR₂. In some embodiments, R^{L1} is —NR₂, —N(R)C(O)OR, or —N(R)C(O)R. In some embodiments, R^{L1} is —NR₂, —N(R)C(O)OR, —N(R)C(O)R, or —N(R)S(O)₂R.

In some embodiments, R^{L1} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}.

In some embodiments, R^{L1} is a C₁₋₆ aliphatic chain substituted by s⁷ instances of R^{L1C} In some embodiments, R^{L1} is phenyl substituted by s⁷ instances of R^{L1C}. In some embodiments, R^{L1} is naphthyl substituted by s⁷ instances of R^{L1C}. In some embodiments, R^{L1} is a 5-6 membered monocyclic heteroaryl ring having 1-4 heteroatoms independently selected from nitrogen, oxygen, and sulfur; wherein said ring is substituted by s⁸ instances of R^{L1C}. In some embodiments, R^{L1} is an 8-10 membered bicyclic heteroaryl ring having 1-4 heteroatoms independently selected from nitrogen, oxygen, and sulfur; wherein said ring is substituted by s⁸ instances of R^{L1C}. In some embodiments, R^{L1} is a 3-7 membered saturated or partially unsaturated monocyclic carbocyclic ring substituted by s⁸ instances of R^{L1C}. In some embodiments, R^{L1} is a 5-12 membered saturated or partially unsaturated bicyclic carbocyclic ring substituted by s⁸ instances of R^{L1C}. In some embodiments, R^{L1} is a 3-7 membered saturated or partially unsaturated monocyclic heterocyclic ring having 1-2 heteroatoms independently selected from nitrogen, oxygen, and sulfur; wherein said ring is substituted by s⁸ instances of R^{L1C}. In some embodiments, R^{L1} is a 7-12 membered saturated or partially unsaturated bicyclic heterocyclic ring having 1-4 heteroatoms independently selected from nitrogen, oxygen, and sulfur; wherein said ring is substituted by s⁷ instances of R^{L1C}.

In some embodiments, R^{L1} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁷ instances of R^{L1C}.

In some embodiments, R^{L1} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁸ instances of R^{L1C}. In some embodiments, R^{L1} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}.

In some embodiments, R^{L1} is phenyl; naphthyl; a 3-7 membered saturated or partially unsaturated monocyclic carbocyclic ring; or a 5-12 membered saturated or partially unsaturated bicyclic carbocyclic ring; each of which is substituted by s⁸ instances of R^{L1C}. In some embodiments, R^{L1} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}.

In some embodiments, R^{L1} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁸ instances of R^{L1C}. In some embodiments, R^{L1} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by s⁷ instances of R^{L1C}.

In some embodiments, R^{L1} is phenyl or naphthyl; each of which is substituted by s⁸ instances of R^{L1C}. In some embodiments, R^{L1} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by s⁷ instances of R^{L1C}. In some embodiments, R^{L1} is a 3-7 membered saturated or partially unsaturated monocyclic carbocyclic ring or a 5-12 membered saturated or partially unsaturated bicyclic carbocyclic ring; each of which is substituted by s⁷ instances of R^{L1C}. In some embodiments, R^{L1} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}.

In some embodiments, R^{L1} is phenyl or a 5-6 membered monocyclic heteroaryl ring having 1-4 heteroatoms independently selected from nitrogen, oxygen, and sulfur; each of which is substituted by s⁸ instances of R^{L1C}. In some embodiments, R^{L1} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by s⁸ instances of R^{L1C}. In some embodiments, R^{L1} is naphthyl or an 8-10 membered bicyclic heteroaryl ring having 1-4 heteroatoms independently selected from nitrogen, oxygen, and sulfur; each of which is substituted by s⁸ instances of R^{L1C}. In some embodiments, R^{L1} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by s⁷ instances of R^{L1C}.

In some embodiments, R^{L1} is phenyl or a 3-7 membered saturated or partially unsaturated monocyclic carbocyclic ring; each of which is substituted by s⁸ instances of R^{L1C} In some embodiments, R^{L1} is naphthyl or a 5-12 membered saturated or partially unsaturated bicyclic carbocyclic ring; each of which is substituted by s⁸ instances of R^{L1C}. In some embodiments, R^{L1} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁸ instances of R^{L1C}. In some embodiments, R^{L1} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}.

In some embodiments, R^{L1} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by s⁸ instances of R^{L1C}. In some embodiments, R^{L1} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by s⁸ instances of R^{L1C}. In some embodiments, R^{L1} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by s⁸ instances of R^{L1C}.

In some embodiments, R_{L1} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by s⁷ instances of R^{L1C}. In some embodiments, R^{L1} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by s⁷ instances of R^{L1C}. In some embodiments, R^{L1} is a C₁₋₆ aliphatic chain, phenyl, or a 3-7 membered saturated or partially unsaturated monocyclic carbocyclic ring; each of which is substituted by s⁸ instances of R^{L1C}.

In some embodiments, R^{L1} is selected from the groups depicted in the compounds in Table 1.

As defined generally above, each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{C} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{C} is oxo. In some embodiments, R^{C} is halogen. In some embodiments, R^{C} is —CN. In some embodiments, R^{C} is —NO₂. In some embodiments, R^{C} is —OR. In some embodiments, R^{C} is —SF₅. In some embodiments, R^{C} is —SR. In some embodiments, R^{C} is —NR₂. In some embodiments, R^{C} is —S(O)₂R. In some embodiments, R^{C} is —S(O)₂NR₂. In some embodiments, R^{C} is —S(O)₂F. In some embodiments, R^{C} is —S(O)R. In some embodiments, R^{C} is —S(O)NR₂. In some embodiments, R^{C} is —S(O)(NR)R. In some embodiments, R^{C} is —C(O)R. In some embodiments, R^{C} is —C(O)OR. In some embodiments, R^{C} is —C(O)NR₂. In some embodiments, R^{C} is —C(O)N(R)OR. In some embodiments, R^{C} is —OC(O)R. In some embodiments, R^{C} is —OC(O)NR₂. In some embodiments, R^{C} is —N(R)C(O)OR. In some embodiments, R^{C} is —N(R)C(O)R. In some embodiments, R^{C} is —N(R)C(O)NR₂. In some embodiments, R^{C} is —N(R)C(NR)NR₂. In some embodiments, R^{C} is —N(R)S(O)₂NR₂. In some embodiments, R^{C} is —N(R)S(O)₂R. In some embodiments, R^{C} is —P(O)R₂. In some embodiments, R^{C} is —P(O)(R)OR. In some embodiments, R^{C} is —B(OR)₂. In some embodiments, R^{C} is deuterium.

In some embodiments, R^{C} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{C} is halogen, —CN, or —NO₂. In some embodiments, R^{C} is —OR, —SR, or —NR₂. In some embodiments, R^{C} is —S(O)₂R, —S(O)₂NR₂, —S(O)₂F, —S(O)R, —S(O)NR₂, or —S(O)(NR)R. In some embodiments, R^{C} is —C(O)R, —C(O)OR, —C(O)NR₂, or —C(O)N(R)OR. In some embodiments, R^{C} is —OC(O)R or —OC(O)NR₂. In some embodiments, R^{C} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{C} is —P(O)R₂ or —P(O)(R)OR.

In some embodiments, R^{C} is —OR, —OC(O)R, or —OC(O)NR₂. In some embodiments, R^{C} is —SR, —S(O)₂R, —S(O)₂NR₂, —S(O)₂F, —S(O)R, —S(O)NR₂, or —S(O)(NR)R. In some embodiments, R^{C} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{C} is —S(O)₂R, —S(O)₂NR₂, or —S(O)₂F. In some embodiments, R^{C} is —S(O)R, —S(O)NR₂, or —S(O)(NR)R. In some embodiments, R^{C} is —SR, —S(O)₂R, or —S(O)R. In some embodiments, R^{C} is —S(O)₂NR₂, —S(O)NR₂, or —S(O)(NR)R. In some embodiments, R^{C} is —S(O)₂NR₂ or —S(O)NR₂. In some embodiments, R^{C} is —SR, —S(O)₂R, —S(O)₂NR₂, or —S(O)R.

In some embodiments, R^{C} is —N(R)C(O)OR, —N(R)C(O)R, or —N(R)C(O)NR₂. In some embodiments, R^{C} is —N(R)S(O)₂NR₂ or —N(R)S(O)₂R. In some embodiments, R^{C} is —N(R)C(O)OR or —N(R)C(O)R. In some embodiments, R^{C} is —N(R)C(O)NR₂ or —N(R)S(O)₂NR₂. In some embodiments, R^{C} is —N(R)C(O)OR, —N(R)C(O)R, or —N(R)S(O)₂R.

In some embodiments, R^{C} is —NR₂, —N(R)C(O)OR, —N(R)C(O)R, or —N(R)C(O)NR₂. In some embodiments, R^{C} is —NR₂, —N(R)C(O)OR, or —N(R)C(O)R. In some embodiments, R^{C} is —NR₂, —N(R)C(O)OR, —N(R)C(O)R, or —N(R)S(O)₂R.

In some embodiments, R^{C} is selected from the groups depicted in the compounds in Table 1.

As defined generally above, each instance of R^{D} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is a C₁₋₆ aliphatic chain. In some embodiments, R^{D} is phenyl. In some embodiments, R^{D} is naphthyl. In some embodiments, R^{D} is cubanyl. In some embodiments, R^{D} is adamantyl. In some embodiments, R^{D} is a 5-6 membered monocyclic heteroaryl ring having 1-4 heteroatoms independently selected from nitrogen, oxygen, and sulfur. In some embodiments, R^{D} is an 8-10 membered bicyclic heteroaryl ring having 1-4 heteroatoms independently selected from nitrogen, oxygen, and sulfur. In some embodiments, R^{D} is a 3-7 membered saturated or partially unsaturated monocyclic carbocyclic ring. In some embodiments, R^{D} is a 5-12 membered saturated or partially unsaturated bicyclic carbocyclic ring. In some embodiments, R^{D} is a 3-7 membered saturated or partially unsaturated monocyclic heterocyclic ring having 1-2 heteroatoms independently selected from nitrogen, oxygen, and sulfur. In some embodiments, R^{D} is a 7-12 membered saturated or partially unsaturated bicyclic heterocyclic ring having 1-4 heteroatoms independently selected from nitrogen, oxygen, and sulfur.

In some embodiments, R^{D} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{D} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{D} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{D} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{D} is phenyl or naphthyl. In some embodiments, R^{D} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{D} is a 3-7 membered saturated or partially unsaturated monocyclic carbocyclic ring or a 5-12 membered saturated or partially unsaturated bicyclic carbocyclic ring. In some embodiments, R^{D} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is phenyl or a 5-6 membered monocyclic heteroaryl ring having 1-4 heteroatoms independently selected from nitrogen, oxygen, and sulfur. In some embodiments, R^{D} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{D} is naphthyl or an 8-10 membered bicyclic heteroaryl ring having 1-4 heteroatoms independently selected from nitrogen, oxygen, and sulfur. In some embodiments, R^{D} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{D} is phenyl or a 3-7 membered saturated or partially unsaturated monocyclic carbocyclic ring. In some embodiments, R^{D} is naphthyl or a 5-12 membered saturated or partially unsaturated bicyclic carbocyclic ring. In some embodiments, R^{D} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{D} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{D} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{D} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{D} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{D} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{D} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{D} is a C₁₋₆ aliphatic chain, phenyl, or a 3-7 membered saturated or partially unsaturated monocyclic carbocyclic ring.

In some embodiments, R^{D} is selected from the groups depicted in the compounds in Table 1.

As defined generally above, each instance of R^{3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{3C} is oxo. In some embodiments, R^{3C} is deuterium. In some embodiments, each instance of R^{3C} is independently halogen. In some embodiments, R^{3C} is —CN. In some embodiments, R^{3C} is —NO₂. In some embodiments, R^{3C} is —OR. In some embodiments, R^{3C} is —SR. In some embodiments, R^{3C} is —NR₂. In some embodiments, R^{3C} is —S(O)₂R. In some embodiments, R^{3C} is —S(O)₂NR₂. In some embodiments, R^{3C} is —S(O)₂F. In some embodiments, R^{3C} is —S(O)R. In some embodiments, R^{3C} is —S(O)NR₂. In some embodiments, R^{3C} is —S(O)(NR)R. In some embodiments, R^{3C} is —C(O)R. In some embodiments, R^{3C} is —C(O)OR. In some embodiments, R^{3C} is —C(O)NR₂. In some embodiments, R^{3C} is —C(O)N(R)OR. In some embodiments, R^{3C} is —OC(O)R. In some embodiments, R^{3C} is —OC(O)NR₂. In some embodiments, R^{3C} is —N(R)C(O)OR. In some embodiments, R^{3C} is —N(R)C(O)R. In some embodiments, R^{3C} is —N(R)C(O)NR₂. In some embodiments, R^{3C} is —N(R)C(NR)NR₂. In some embodiments, R^{3C} is —N(R)S(O)₂NR₂. In some embodiments, R^{3C} is —N(R)S(O)₂R. In some embodiments, R^{3C} is —P(O)R₂. In some embodiments, R^{3C} is —P(O)(R)OR. In some embodiments, R^{3C} is —B(OR)₂.

In some embodiments, each instance of R^{3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{3C} is independently halogen, —CN, or —NO₂. In some embodiments, each instance of R^{3C} is independently —OR, —SR, or —NR₂. In some embodiments, each instance of R^{3C} is independently —S(O)₂R, —S(O)₂NR₂, —S(O)₂F, —S(O)R, —S(O)NR₂, or —S(O)(NR)R. In some embodiments, each instance of R^{3C} is independently —C(O)R, —C(O)OR, —C(O)NR₂, or —C(O)N(R)OR. In some embodiments, each instance of R^{3C} is independently —OC(O)R or —OC(O)NR₂. In some embodiments, each instance of R^{3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{3C} is independently —P(O)R₂ or —P(O)(R)OR.

In some embodiments, each instance of R^{3C} is independently —OR, —OC(O)R, or —OC(O)NR₂. In some embodiments, each instance of R^{3C} is independently —SR, —S(O)₂R, —S(O)₂NR₂, —S(O)₂F, —S(O)R, —S(O)NR₂, or —S(O)(NR)R. In some embodiments, each instance of R^{3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{3C} is independently —S(O)₂R, —S(O)₂NR₂, or —S(O)₂F. In some embodiments, each instance of R^{3C} is independently —S(O)R, —S(O)NR₂, or —S(O)(NR)R. In some embodiments, each instance of R^{3C} is independently —SR, —S(O)₂R, or —S(O)R. In some embodiments, each instance of R^{3C} is independently —S(O)₂NR₂, —S(O)NR₂, or —S(O)(NR)R. In some embodiments, each instance of R^{3C} is independently —S(O)₂NR₂ or —S(O)NR₂. In some embodiments, each instance of R^{3C} is independently —SR, —S(O)₂R, —S(O)₂NR₂, or —S(O)R.

In some embodiments, each instance of R^{3C} is independently —N(R)C(O)OR, —N(R)C(O)R, or —N(R)C(O)NR₂. In some embodiments, each instance of R^{3C} is independently —N(R)S(O)₂NR₂ or —N(R)S(O)₂R. In some embodiments, each instance of R^{3C} is independently —N(R)C(O)OR or —N(R)C(O)R. In some embodiments, each instance of R^{3C} is independently —N(R)C(O)NR₂ or —N(R)S(O)₂NR₂. In some embodiments, each instance of R^{3C} is independently —N(R)C(O)OR, —N(R)C(O)R, or —N(R)S(O)₂R.

In some embodiments, each instance of R^{3C} is independently —NR₂, —N(R)C(O)OR, —N(R)C(O)R, or —N(R)C(O)NR₂. In some embodiments, each instance of R^{3C} is independently —NR₂, —N(R)C(O)OR, or —N(R)C(O)R. In some embodiments, each instance of R^{3C} is independently —NR₂, —N(R)C(O)OR, —N(R)C(O)R, or —N(R)S(O)₂R.

In some embodiments, each instance of R^{3C} is independently an optionally substituted C₁₋₆ aliphatic. In some embodiments, each instance of R^{3C} is independently an optionally substituted phenyl. In some embodiments, each instance of R^{3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{3C} is independently an optionally substituted 5-6 membered monocyclic heteroaryl ring having 1-4 heteroatoms independently selected from nitrogen, oxygen, and sulfur.

In some embodiments, each instance of R^{3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{3C} is independently an optionally substituted C₁₋₆ aliphatic or an optionally substituted phenyl. In some embodiments, each instance of R^{3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{3C} is independently a C₁₋₆ aliphatic. In some embodiments, R^{3C} is phenyl. In some embodiments, each instance of R^{3C} is independently a 3-7 membered saturated or partially unsaturated monocyclic heterocyclic ring having 1-2 heteroatoms independently selected from nitrogen, oxygen, and sulfur. In some embodiments, each instance of R^{3C} is independently a 5-6 membered monocyclic heteroaryl ring having 1-4 heteroatoms independently selected from nitrogen, oxygen, and sulfur.

In some embodiments, each instance of R^{3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{3C} is independently phenyl or a 5-6 membered monocyclic heteroaryl ring having 1-4 heteroatoms independently selected from nitrogen, oxygen, and sulfur.

In some embodiments, each instance of R^{3C} is independently a C₁₋₆ aliphatic or phenyl. In some embodiments, each instance of R^{3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{3C} is independently halogen, —CN, —O-(optionally substituted C₁₋₆ aliphatic), or an optionally substituted C₁₋₆ aliphatic. In some embodiments, each instance of R^{3C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{3C} is independently halogen or C₁₋₃ aliphatic optionally substituted with 1-3 halogen. In some embodiments, each instance of R^{3C} is independently fluorine, chlorine, —CH₃, —CHF₂, or —CF₃.

In some embodiments, each instance of R^{3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{3C} is independently selected from the groups depicted in the compounds in Table 1.

As defined generally above, each instance of R^{4C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{4C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{4C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{4C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{4C} is oxo. In some embodiments, R^{4C} is deuterium. In some embodiments, each instance of R^{4C} is independently halogen. In some embodiments, R^{4C} is —CN. In some embodiments, R^{4C} is —NO₂. In some embodiments, R^{4C} is —OR. In some embodiments, R^{4C} is —SR. In some embodiments, R^{4C} is —NR₂. In some embodiments, R^{4C} is —S(O)₂R. In some embodiments, R^{4C} is —S(O)₂NR₂. In some embodiments, R^{4C} is —S(O)₂F. In some embodiments, R^{4C} is —S(O)R. In some embodiments, R^{4C} is —S(O)NR₂. In some embodiments, R^{4C} is —S(O)(NR)R. In some embodiments, R^{4C} is —C(O)R. In some embodiments, R^{4C} is —C(O)OR. In some embodiments, R^{4C} is —C(O)NR₂. In some embodiments, R^{4C} is —C(O)N(R)OR. In some embodiments, R^{4C} is —OC(O)R. In some embodiments, R^{4C} is —OC(O)NR₂. In some embodiments, R^{4C} is —N(R)C(O)OR. In some embodiments, R^{4C} is —N(R)C(O)R. In some embodiments, R^{4C} is —N(R)C(O)NR₂. In some embodiments, R^{4C} is —N(R)C(NR)NR₂. In some embodiments, R^{4C} is —N(R)S(O)₂NR₂. In some embodiments, R^{4C} is —N(R)S(O)₂R. In some embodiments, R^{4C} is —P(O)R₂. In some embodiments, R^{4C} is —P(O)(R)OR. In some embodiments, R^{4C} is —B(OR)₂.

In some embodiments, each instance of R^{4C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{4C} is independently halogen, —CN, or —NO₂. In some embodiments, each instance of R^{4C} is independently —OR, —SR, or —NR₂. In some embodiments, each instance of R^{4C} is independently —S(O)₂R, —S(O)₂NR₂, —S(O)₂F, —S(O)R, —S(O)NR₂, or —S(O)(NR)R. In some embodiments, each instance of R^{4C} is independently —C(O)R, —C(O)OR, —C(O)NR₂, or —C(O)N(R)OR. In some embodiments, each instance of R^{4C} is independently —OC(O)R or —OC(O)NR₂. In some embodiments, each instance of R^{4C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{4C} is independently —P(O)R₂ or —P(O)(R)OR.

In some embodiments, each instance of R^{4C} is independently —OR, —OC(O)R, or —OC(O)NR₂. In some embodiments, each instance of R^{4C} is independently —SR, —S(O)₂R, —S(O)₂NR₂, —S(O)₂F, —S(O)R, —S(O)NR₂, or —S(O)(NR)R. In some embodiments, each instance of R^{4C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{4C} is independently —S(O)₂R, —S(O)₂NR₂, or —S(O)₂F. In some embodiments, each instance of R^{4C} is independently —S(O)R, —S(O)NR₂, or —S(O)(NR)R. In some embodiments, each instance of R^{4C} is independently —SR, —S(O)₂R, or —S(O)R. In some embodiments, each instance of R^{4C} is independently —S(O)₂NR₂, —S(O)NR₂, or —S(O)(NR)R. In some embodiments, each instance of R^{4C} is independently —S(O)₂NR₂ or —S(O)NR₂. In some embodiments, each instance of R^{4C} is independently —SR, —S(O)₂R, —S(O)₂NR₂, or —S(O)R.

In some embodiments, each instance of R^{4C} is independently —N(R)C(O)OR, —N(R)C(O)R, or —N(R)C(O)NR₂. In some embodiments, each instance of R^{4C} is independently —N(R)S(O)₂NR₂ or —N(R)S(O)₂R. In some embodiments, each instance of R^{4C} is independently —N(R)C(O)OR or —N(R)C(O)R. In some embodiments, each instance of R^{4C} is independently —N(R)C(O)NR₂ or —N(R)S(O)₂NR₂. In some embodiments, each instance of R^{4C} is independently —N(R)C(O)OR, —N(R)C(O)R, or —N(R)S(O)₂R.

In some embodiments, each instance of R^{4C} is independently —NR₂, —N(R)C(O)OR, —N(R)C(O)R, or —N(R)C(O)NR₂. In some embodiments, each instance of R^{4C} is independently —NR₂, —N(R)C(O)OR, or —N(R)C(O)R. In some embodiments, each instance of R^{4C} is independently —NR₂, —N(R)C(O)OR, —N(R)C(O)R, or —N(R)S(O)₂R.

In some embodiments, each instance of R^{4C} is independently an optionally substituted C₁₋₆ aliphatic. In some embodiments, each instance of R^{4C} is independently an optionally substituted phenyl. In some embodiments, each instance of R^{4C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{4C} is independently an optionally substituted 5-6 membered monocyclic heteroaryl ring having 1-4 heteroatoms independently selected from nitrogen, oxygen, and sulfur.

In some embodiments, each instance of R^{4C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{4C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{4C} is independently an optionally substituted C₁₋₆ aliphatic or an optionally substituted phenyl. In some embodiments, each instance of R^{4C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{4C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{4C} is independently a C₁₋₆ aliphatic. In some embodiments, R^{4C} is phenyl. In some embodiments, each instance of R^{4C} is independently a 3-7 membered saturated or partially unsaturated monocyclic heterocyclic ring having 1-2 heteroatoms independently selected from nitrogen, oxygen, and sulfur. In some embodiments, each instance of R^{4C} is independently a 5-6 membered monocyclic heteroaryl ring having 1-4 heteroatoms independently selected from nitrogen, oxygen, and sulfur.

In some embodiments, each instance of R^{4C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{4C} is independently phenyl or a 5-6 membered monocyclic heteroaryl ring having 1-4 heteroatoms independently selected from nitrogen, oxygen, and sulfur.

In some embodiments, each instance of R^{4C} is independently a C₁₋₆ aliphatic or phenyl. In some embodiments, each instance of R^{4C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and

In some embodiments, each instance of R^{4C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{4C} is independently halogen, —CN, —O-(optionally substituted C₁₋₆ aliphatic), or an optionally substituted C₁₋₆ aliphatic. In some embodiments, each instance of R^{4C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{4C} is independently halogen or C₁₋₃ aliphatic optionally substituted with 1-3 halogen. In some embodiments, each instance of R^{4C} is independently fluorine, chlorine, —CH₃, —CHF₂, or —CF₃.

In some embodiments, each instance of R^{4C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{4C} is independently selected from the groups depicted in the compounds in Table 1.

As defined generally above, each instance of R^{EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R) C(O)R, —N(R)C(O)NR₂, —N(R)C(NR)NR₂, —N(R)S(O)₂NR₂, —N(R)S(O)₂R, —P(O)R₂, —P(O)(R) OR, or —B(OR)₂. In some embodiments, each instance of R^{EC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{EC} is independently deuterium, halogen, —CN, —OR, or —NR₂. In some embodiments, each instance of R^{EC} is independently deuterium or halogen. In some embodiments, each instance of R^{EC} is independently halogen.

In some embodiments, each instance of R^{EC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EC} is independently selected from the groups depicted in the compounds in Table 1.

As defined generally above, each instance of R^{QC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{QC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{QC} is oxo. In some embodiments, R^{QC} is deuterium. In some embodiments, each instance of R^{QC} is independently halogen. In some embodiments, R^{QC} is —CN. In some embodiments, R^{QC} is —NO₂. In some embodiments, R^{QC} is —OR. In some embodiments, R^{QC} is —SR. In some embodiments, R^{QC} is —NR₂. In some embodiments, R^{QC} is —S(O)₂R. In some embodiments, R^{QC} is —S(O)₂NR₂. In some embodiments, R^{QC} is —S(O)₂F. In some embodiments, R^{QC} is —S(O)R. In some embodiments, R^{QC} is —S(O)NR₂. In some embodiments, R^{QC} is —S(O)(NR)R. In some embodiments, R^{QC} is —C(O)R. In some embodiments, R^{QC} is —C(O)OR. In some embodiments, R^{QC} is —C(O)NR₂. In some embodiments, R^{QC} is —C(O)N(R)OR. In some embodiments, R^{QC} is —OC(O)R. In some embodiments, R^{QC} is —OC(O)NR₂. In some embodiments, R^{QC} is —N(R)C(O)OR. In some embodiments, R^{QC} is —N(R)C(O)R. In some embodiments, R^{QC} is —N(R)C(O)NR₂. In some embodiments, R^{QC} is —N(R)C(NR)NR₂. In some embodiments, R^{QC} is —N(R)S(O)₂NR₂. In some embodiments, R^{QC} is —N(R)S(O)₂R. In some embodiments, R^{QC} is —P(O)R₂. In some embodiments, R^{QC} is —P(O)(R)OR. In some embodiments, R^{QC} is —B(OR)₂.

In some embodiments, each instance of R^{QC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{QC} is independently halogen, —CN, or —NO₂. In some embodiments, each instance of R^{QC} is independently —OR, —SR, or —NR₂. In some embodiments, each instance of R^{QC} is independently —S(O)₂R, —S(O)₂NR₂, —S(O)₂F, —S(O)R, —S(O)NR₂, or —S(O)(NR)R. In some embodiments, each instance of R^{QC} is independently —C(O)R, —C(O)OR, —C(O)NR₂, or —C(O)N(R)OR. In some embodiments, each instance of R^{QC} is independently —OC(O)R or —OC(O)NR₂. In some embodiments, each instance of R^{QC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{QC} is independently —P(O)R₂ or —P(O)(R)OR.

In some embodiments, each instance of R^{QC} is independently —OR, —OC(O)R, or —OC(O)NR₂. In some embodiments, each instance of R^{QC} is independently —SR, —S(O)₂R, —S(O)₂NR₂, —S(O)₂F, —S(O)R, —S(O)NR₂, or —S(O)(NR)R. In some embodiments, each instance of R^{QC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{QC} is independently —S(O)₂R, —S(O)₂NR₂, or —S(O)₂F. In some embodiments, each instance of R^{QC} is independently —S(O)R, —S(O)NR₂, or —S(O)(NR)R. In some embodiments, each instance of R^{QC} is independently —SR, —S(O)₂R, or —S(O)R. In some embodiments, each instance of R^{QC} is independently —S(O)₂NR₂, —S(O)NR₂, or —S(O)(NR)R. In some embodiments, each instance of R^{QC} is independently —S(O)₂NR₂ or —S(O)NR₂. In some embodiments, each instance of R^{QC} is independently —SR, —S(O)₂R, —S(O)₂NR₂, or —S(O)R.

In some embodiments, each instance of R^{QC} is independently —N(R)C(O)OR, —N(R)C(O)R, or —N(R)C(O)NR₂. In some embodiments, each instance of R^{QC} is independently —N(R)S(O)₂NR₂ or —N(R)S(O)₂R. In some embodiments, each instance of R^{QC} is independently —N(R)C(O)OR or —N(R)C(O)R. In some embodiments, each instance of R^{QC} is independently —N(R)C(O)NR₂ or —N(R)S(O)₂NR₂. In some embodiments, each instance of R^{QC} is independently —N(R)C(O)OR, —N(R)C(O)R, or —N(R)S(O)₂R.

In some embodiments, each instance of R^{QC} is independently —NR₂, —N(R)C(O)OR, —N(R)C(O)R, or —N(R)C(O)NR₂. In some embodiments, each instance of R^{QC} is independently —NR₂, —N(R)C(O)OR, or —N(R)C(O)R. In some embodiments, each instance of R^{QC} is independently —NR₂, —N(R)C(O)OR, —N(R)C(O)R, or —N(R)S(O)₂R.

In some embodiments, each instance of R^{QC} is independently an optionally substituted C₁₋₆ aliphatic. In some embodiments, each instance of R^{QC} is independently an optionally substituted phenyl. In some embodiments, each instance of R^{QC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{QC} is independently an optionally substituted 5-6 membered monocyclic heteroaryl ring having 1-4 heteroatoms independently selected from nitrogen, oxygen, and sulfur.

In some embodiments, each instance of R^{QC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{QC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{QC} is independently an optionally substituted C₁₋₆ aliphatic or an optionally substituted phenyl. In some embodiments, each instance of R^{QC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{QC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic. In some embodiments, R^{QC} is phenyl. In some embodiments, each instance of R^{QC} is independently a 3-7 membered saturated or partially unsaturated monocyclic heterocyclic ring having 1-2 heteroatoms independently selected from nitrogen, oxygen, and sulfur. In some embodiments, each instance of R^{QC} is independently a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{QC} is independently phenyl or a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic or phenyl. In some embodiments, each instance of R^{QC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{QC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{QC} is independently selected from the groups depicted in the compounds in Table 1.

As defined generally above, each instance of R^{Z1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Z1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Z1C} is oxo. In some embodiments, R^{Z1C} is deuterium. In some embodiments, each instance of R^{Z1C} is independently halogen. In some embodiments, R^{Z1C} is —CN. In some embodiments, R^{Z1C} is —NO₂. In some embodiments, R^{Z1C} is —OR. In some embodiments, R^{Z1C} is —SR. In some embodiments, R^{Z1C} is —NR₂. In some embodiments, R^{Z1C} is —S(O)₂R. In some embodiments, R^{Z1C} is —S(O)₂NR₂. In some embodiments, R^{Z1C} is —S(O)₂F. In some embodiments, R^{Z1C} is —S(O)R. In some embodiments, R^{Z1C} is —S(O)NR₂. In some embodiments, R^{Z1C} is —S(O)(NR)R. In some embodiments, R^{Z1C} is —C(O)R. In some embodiments, R^{Z1C} is —C(O)OR. In some embodiments, R^{Z1C} is —C(O)NR₂. In some embodiments, R^{Z1C} is —C(O)N(R)OR. In some embodiments, R^{Z1C} is —OC(O)R. In some embodiments, R^{Z1C} is —OC(O)NR₂. In some embodiments, R^{Z1C} is —N(R)C(O)OR. In some embodiments, R^{Z1C} is —N(R)C(O)R. In some embodiments, R^{Z1C} is —N(R)C(O)NR₂. In some embodiments, R^{Z1C} is —N(R)C(NR)NR₂. In some embodiments, R^{Z1C} is —N(R)S(O)₂NR₂. In some embodiments, R^{Z1C} is —N(R)S(O)₂R. In some embodiments, R^{Z1C} is —P(O)R₂. In some embodiments, R^{Z1C} is —P(O)(R)OR. In some embodiments, R^{Z1C} is —B(OR)₂.

In some embodiments, each instance of R^{Z1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Z1C} is independently halogen, —CN, or —NO₂. In some embodiments, each instance of R^{Z1C} is independently —OR, —SR, or —NR₂. In some embodiments, each instance of R^{Z1C} is independently —S(O)₂R, —S(O)₂NR₂, —S(O)₂F, —S(O)R, —S(O)NR₂, or —S(O)(NR)R. In some embodiments, each instance of R^{Z1C} is independently —C(O)R, —C(O)OR, —C(O)NR₂, or —C(O)N(R)OR. In some embodiments, each instance of R^{Z1C} is independently —OC(O)R or —OC(O)NR₂. In some embodiments, each instance of R^{Z1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Z1C} is independently —P(O)R₂ or —P(O)(R)OR.

In some embodiments, each instance of R^{Z1C} is independently —OR, —OC(O)R, or —OC(O)NR₂. In some embodiments, each instance of R^{Z1C} is independently —SR, —S(O)₂R, —S(O)₂NR₂, —S(O)₂F, —S(O)R, —S(O)NR₂, or —S(O)(NR)R. In some embodiments, each instance of R^{Z1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Z1C} is independently —S(O)₂R, —S(O)₂NR₂, or —S(O)₂F. In some embodiments, each instance of R^{Z1C} is independently —S(O)R, —S(O)NR₂, or —S(O)(NR)R. In some embodiments, each instance of R^{Z1C} is independently —SR, —S(O)₂R, or —S(O)R. In some embodiments, each instance of R^{Z1C} is independently —S(O)₂NR₂, —S(O)NR₂, or —S(O)(NR)R. In some embodiments, each instance of R^{Z1C} is independently —S(O)₂NR₂ or —S(O)NR₂. In some embodiments, each instance of R^{Z1C} is independently —SR, —S(O)₂R, —S(O)₂NR₂, or —S(O)R.

In some embodiments, each instance of R^{Z1C} is independently —N(R)C(O)OR, —N(R)C(O)R, or —N(R)C(O)NR₂. In some embodiments, each instance of R^{Z1C} is independently —N(R)S(O)₂NR₂ or —N(R)S(O)₂R. In some embodiments, each instance of R^{Z1C} is independently —N(R)C(O)OR or —N(R)C(O)R. In some embodiments, each instance of R^{Z1C} is independently —N(R)C(O)NR₂ or —N(R)S(O)₂NR₂. In some embodiments, each instance of R^{Z1C} is independently —N(R)C(O)OR, —N(R)C(O)R, or —N(R)S(O)₂R.

In some embodiments, each instance of R^{Z1C} is independently —NR₂, —N(R)C(O)OR, —N(R)C(O)R, or —N(R)C(O)NR₂. In some embodiments, each instance of R^{Z1C} is independently —NR₂, —N(R)C(O)OR, or —N(R)C(O)R. In some embodiments, each instance of R^{Z1C} is independently —NR₂, —N(R)C(O)OR, —N(R)C(O)R, or —N(R)S(O)₂R.

In some embodiments, each instance of R^{Z1C} is independently an optionally substituted C₁₋₆ aliphatic. In some embodiments, each instance of R^{Z1C} is independently an optionally substituted phenyl. In some embodiments, each instance of R^{Z1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Z1C} is independently an optionally substituted 5-6 membered monocyclic heteroaryl ring having 1-4 heteroatoms independently selected from nitrogen, oxygen, and sulfur.

In some embodiments, each instance of R^{Z1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Z1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Z1C} is independently an optionally substituted C₁₋₆ aliphatic or an optionally substituted phenyl. In some embodiments, each instance of R^{Z1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Z1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z1C} is independently a C₁₋₆ aliphatic. In some embodiments, R^{Z1C} is phenyl. In some embodiments, each instance of R^{Z1C} is independently a 3-7 membered saturated or partially unsaturated monocyclic heterocyclic ring having 1-2 heteroatoms independently selected from nitrogen, oxygen, and sulfur. In some embodiments, each instance of R^{Z1C} is independently a 5-6 membered monocyclic heteroaryl ring having 1-4 heteroatoms independently selected from nitrogen, oxygen, and sulfur.

In some embodiments, each instance of R^{Z1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Z1C} is independently phenyl or a 5-6 membered monocyclic heteroaryl ring having 1-4 heteroatoms independently selected from nitrogen, oxygen, and sulfur.

In some embodiments, each instance of R^{Z1C} is independently a C₁₋₆ aliphatic or phenyl. In some embodiments, each instance of R^{Z1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z1C} is independently selected from the groups depicted in the compounds in Table 1.

As defined generally above, each instance of R^{Z2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Z2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Z2C} is oxo. In some embodiments, R^{Z2C} is deuterium. In some embodiments, each instance of R^{Z2C} is independently halogen. In some embodiments, R^{Z2C} is —CN. In some embodiments, R^{Z2C} is —NO₂. In some embodiments, R^{Z2C} is —OR. In some embodiments, R^{Z2C} is —SR. In some embodiments, R^{Z2C} is —NR₂. In some embodiments, R^{Z2C} is —S(O)₂R. In some embodiments, R^{Z2C} is —S(O)₂NR₂. In some embodiments, R^{Z2C} is —S(O)₂F. In some embodiments, R^{Z2C} is —S(O)R. In some embodiments, R^{Z2C} is —S(O)NR₂. In some embodiments, R^{Z2C} is —S(O)(NR)R. In some embodiments, R^{Z2C} is —C(O)R. In some embodiments, R^{Z2C} is —C(O)OR. In some embodiments, R^{Z2C} is —C(O)NR₂. In some embodiments, R^{Z2C} is —C(O)N(R)OR. In some embodiments, R^{Z2C} is —OC(O)R. In some embodiments, R^{Z2C} is —OC(O)NR₂. In some embodiments, R^{Z2C} is —N(R)C(O)OR. In some embodiments, R^{Z2C} is —N(R)C(O)R. In some embodiments, R^{Z2C} is —N(R)C(O)NR₂. In some embodiments, R^{Z2C} is —N(R)C(NR)NR₂. In some embodiments, R^{Z2C} is —N(R)S(O)₂NR₂. In some embodiments, R^{Z2C} is —N(R)S(O)₂R. In some embodiments, R^{Z2C} is —P(O)R₂. In some embodiments, R^{Z2C} is —P(O)(R)OR. In some embodiments, R^{Z2C} is —B(OR)₂.

In some embodiments, each instance of R^{Z2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Z2C} is independently halogen, —CN, or —NO₂. In some embodiments, each instance of R^{Z2C} is independently —OR, —SR, or —NR₂. In some embodiments, each instance of R^{Z2C} is independently —S(O)₂R, —S(O)₂NR₂, —S(O)₂F, —S(O)R, —S(O)NR₂, or —S(O)(NR)R. In some embodiments, each instance of R^{Z2C} is independently —C(O)R, —C(O)OR, —C(O)NR₂, or —C(O)N(R)OR. In some embodiments, each instance of R^{Z2C} is independently —OC(O)R or —OC(O)NR₂. In some embodiments, each instance of R^{Z2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Z2C} is independently —P(O)R₂ or —P(O)(R)OR.

In some embodiments, each instance of R^{Z2C} is independently —OR, —OC(O)R, or —OC(O)NR₂. In some embodiments, each instance of R^{Z2C} is independently —SR, —S(O)₂R, —S(O)₂NR₂, —S(O)₂F, —S(O)R, —S(O)NR₂, or —S(O)(NR)R. In some embodiments, each instance of R^{Z2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Z2C} is independently —S(O)₂R, —S(O)₂NR₂, or —S(O)₂F. In some embodiments, each instance of R^{Z2C} is independently —S(O)R, —S(O)NR₂, or —S(O)(NR)R. In some embodiments, each instance of R^{Z2C} is independently —SR, —S(O)₂R, or —S(O)R. In some embodiments, each instance of R^{Z2C} is independently —S(O)₂NR₂, —S(O)NR₂, or —S(O)(NR)R. In some embodiments, each instance of R^{Z2C} is independently —S(O)₂NR₂ or —S(O)NR₂. In some embodiments, each instance of R^{Z2C} is independently —SR, —S(O)₂R, —S(O)₂NR₂, or —S(O)R.

In some embodiments, each instance of R^{Z2C} is independently —N(R)C(O)OR, —N(R)C(O)R, or —N(R)C(O)NR₂. In some embodiments, each instance of R^{Z2C} is independently —N(R)S(O)₂NR₂ or —N(R)S(O)₂R. In some embodiments, each instance of R^{Z2C} is independently —N(R)C(O)OR or —N(R)C(O)R. In some embodiments, each instance of R^{Z2C} is independently —N(R)C(O)NR₂ or —N(R)S(O)₂NR₂. In some embodiments, each instance of R^{Z2C} is independently —N(R)C(O)OR, —N(R)C(O)R, or —N(R)S(O)₂R.

In some embodiments, each instance of R^{Z2C} is independently —NR₂, —N(R)C(O)OR, —N(R)C(O)R, or —N(R)C(O)NR₂. In some embodiments, each instance of R^{Z2C} is independently —NR₂, —N(R)C(O)OR, or —N(R)C(O)R. In some embodiments, each instance of R^{Z2C} is independently —NR₂, —N(R)C(O)OR, —N(R)C(O)R, or —N(R)S(O)₂R.

In some embodiments, each instance of R^{Z2C} is independently an optionally substituted C₁₋₆ aliphatic. In some embodiments, each instance of R^{Z2C} is independently an optionally substituted phenyl. In some embodiments, each instance of R^{Z2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Z2C} is independently an optionally substituted 5-6 membered monocyclic heteroaryl ring having 1-4 heteroatoms independently selected from nitrogen, oxygen, and sulfur.

In some embodiments, each instance of R^{Z2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Z2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Z2C} is independently an optionally substituted C₁₋₆ aliphatic or an optionally substituted phenyl. In some embodiments, each instance of R^{Z2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Z2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2C} is independently a C₁₋₆ aliphatic. In some embodiments, R^{Z2C} is phenyl. In some embodiments, each instance of R^{Z2C} is independently a 3-7 membered saturated or partially unsaturated monocyclic heterocyclic ring having 1-2 heteroatoms independently selected from nitrogen, oxygen, and sulfur. In some embodiments, each instance of R^{Z2C} is independently a 5-6 membered monocyclic heteroaryl ring having 1-4 heteroatoms independently selected from nitrogen, oxygen, and sulfur.

In some embodiments, each instance of R^{Z2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Z2C} is independently phenyl or a 5-6 membered monocyclic heteroaryl ring having 1-4 heteroatoms independently selected from nitrogen, oxygen, and sulfur.

In some embodiments, each instance of R^{Z2C} is independently a C₁₋₆ aliphatic or phenyl. In some embodiments, each instance of R^{Z2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{Z2C} is independently halogen, —CN, —OH, —O-(optionally substituted C₁₋₃ aliphatic), or an optionally substituted C₁₋₃ aliphatic. In some embodiments, each instance of R^{Z2C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{Z2C} is independently fluorine, chlorine, —OH, —OCH₃, —OCF₃, —CH₃, —CHF₂, or —CF₃. In some embodiments, each instance of R^{Z2C} is independently fluorine or —OH.

In some embodiments, each instance of R^{Z2C} is independently oxo, halogen, —CN, —OH, —O-(optionally substituted C₁₋₃ aliphatic), or an optionally substituted C₁₋₃ aliphatic. In some embodiments, each instance of R^{Z2C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{Z2C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{Z2C} is independently oxo, fluorine, chlorine, —CN, —OH, —OCH₃, —OCF₃, —CH₃, —CHF₂, or —CF₃. In some embodiments, each instance of R^{Z2C} is independently oxo, —CN, fluorine, or —OH. In some embodiments, each instance of R^{Z2C} is independently oxo, —CN, —CH₃, or —CHF₂. In some embodiments, each instance of R^{Z2C} is independently —CN, —CH₃, or —CHF₂.

In some embodiments, each instance of R^{Z2C} is independently selected from the groups depicted in the compounds in Table 1.

As defined generally above, each instance of R^{Z3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Z3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Z3C} is oxo. In some embodiments, R^{Z3C} is deuterium. In some embodiments, each instance of R^{Z3C} is independently halogen. In some embodiments, R^{Z3C} is —CN. In some embodiments, R^{Z3C} is —NO₂. In some embodiments, R^{Z3C} is —OR. In some embodiments, R^{Z3C} is —SR. In some embodiments, R^{Z3C} is —NR₂. In some embodiments, R^{Z3C} is —S(O)₂R. In some embodiments, R^{Z3C} is —S(O)₂NR₂. In some embodiments, R^{Z3C} is —S(O)₂F. In some embodiments, R^{Z3C} is —S(O)R. In some embodiments, R^{Z3C} is —S(O)NR₂. In some embodiments, R^{Z3C} is —S(O)(NR)R. In some embodiments, R^{Z3C} is —C(O)R. In some embodiments, R^{Z3C} is —C(O)OR. In some embodiments, R^{Z3C} is —C(O)NR₂. In some embodiments, R^{Z3C} is —C(O)N(R)OR. In some embodiments, R^{Z3C} is —OC(O)R. In some embodiments, R^{Z3C} is —OC(O)NR₂. In some embodiments, R^{Z3C} is —N(R)C(O)OR. In some embodiments, R^{Z3C} is —N(R)C(O)R. In some embodiments, R^{Z3C} is —N(R)C(O)NR₂. In some embodiments, R^{Z3C} is —N(R)C(NR)NR₂. In some embodiments, R^{Z3C} is —N(R)S(O)₂NR₂. In some embodiments, R^{Z3C} is —N(R)S(O)₂R. In some embodiments, R^{Z3C} is —P(O)R₂. In some embodiments, R^{Z3C} is —P(O)(R)OR. In some embodiments, R^{Z3C} is —B(OR)₂.

In some embodiments, each instance of R^{Z3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Z3C} is independently halogen, —CN, or —NO₂. In some embodiments, each instance of R^{Z3C} is independently —OR, —SR, or —NR₂. In some embodiments, each instance of R^{Z3C} is independently —S(O)₂R, —S(O)₂NR₂, —S(O)₂F, —S(O)R, —S(O)NR₂, or —S(O)(NR)R. In some embodiments, each instance of R^{Z3C} is independently —C(O)R, —C(O)OR, —C(O)NR₂, or —C(O)N(R)OR. In some embodiments, each instance of R^{Z3C} is independently —OC(O)R or —OC(O)NR₂. In some embodiments, each instance of R^{Z3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Z3C} is independently —P(O)R₂ or —P(O)(R)OR.

In some embodiments, each instance of R^{Z3C} is independently —OR, —OC(O)R, or —OC(O)NR₂. In some embodiments, each instance of R^{Z3C} is independently —SR, —S(O)₂R, —S(O)₂NR₂, —S(O)₂F, —S(O)R, —S(O)NR₂, or —S(O)(NR)R. In some embodiments, each instance of R^{Z3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Z3C} is independently —S(O)₂R, —S(O)₂NR₂, or —S(O)₂F. In some embodiments, each instance of R^{Z3C} is independently —S(O)R, —S(O)NR₂, or —S(O)(NR)R. In some embodiments, each instance of R^{Z3C} is independently —SR, —S(O)₂R, or —S(O)R. In some embodiments, each instance of R^{Z3C} is independently —S(O)₂NR₂, —S(O)NR₂, or —S(O)(NR)R. In some embodiments, each instance of R^{Z3C} is independently —S(O)₂NR₂ or —S(O)NR₂. In some embodiments, each instance of R^{Z3C} is independently —SR, —S(O)₂R, —S(O)₂NR₂, or —S(O)R.

In some embodiments, each instance of R^{Z3C} is independently —N(R)C(O)OR, —N(R)C(O)R, or —N(R)C(O)NR₂. In some embodiments, each instance of R^{Z3C} is independently —N(R)S(O)₂NR₂ or —N(R)S(O)₂R. In some embodiments, each instance of R^{Z3C} is independently —N(R)C(O)OR or —N(R)C(O)R. In some embodiments, each instance of R^{Z3C} is independently —N(R)C(O)NR₂ or —N(R)S(O)₂NR₂. In some embodiments, each instance of R^{Z3C} is independently —N(R)C(O)OR, —N(R)C(O)R, or —N(R)S(O)₂R.

In some embodiments, each instance of R^{Z3C} is independently —NR₂, —N(R)C(O)OR, —N(R)C(O)R, or —N(R)C(O)NR₂. In some embodiments, each instance of R^{Z3C} is independently —NR₂, —N(R)C(O)OR, or —N(R)C(O)R. In some embodiments, each instance of R^{Z3C} is independently —NR₂, —N(R)C(O)OR, —N(R)C(O)R, or —N(R)S(O)₂R.

In some embodiments, each instance of R^{Z3C} is independently an optionally substituted C₁₋₆ aliphatic. In some embodiments, each instance of R^{Z3C} is independently an optionally substituted phenyl. In some embodiments, each instance of R^{Z3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Z3C} is independently an optionally substituted 5-6 membered monocyclic heteroaryl ring having 1-4 heteroatoms independently selected from nitrogen, oxygen, and sulfur.

In some embodiments, each instance of R^{Z3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Z3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Z3C} is independently an optionally substituted C₁₋₆ aliphatic or an optionally substituted phenyl. In some embodiments, each instance of R^{Z3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Z3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z3C} is independently a C₁₋₆ aliphatic. In some embodiments, R^{Z3C} is phenyl. In some embodiments, each instance of R^{Z3C} is independently a 3-7 membered saturated or partially unsaturated monocyclic heterocyclic ring having 1-2 heteroatoms independently selected from nitrogen, oxygen, and sulfur. In some embodiments, each instance of R^{Z3C} is independently a 5-6 membered monocyclic heteroaryl ring having 1-4 heteroatoms independently selected from nitrogen, oxygen, and sulfur.

In some embodiments, each instance of R^{Z3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Z3C} is independently phenyl or a 5-6 membered monocyclic heteroaryl ring having 1-4 heteroatoms independently selected from nitrogen, oxygen, and sulfur.

In some embodiments, each instance of R^{Z3C} is independently a C₁₋₆ aliphatic or phenyl. In some embodiments, each instance of R^{Z3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{Z3C} is independently halogen, —CN, —OH, —O-(optionally substituted C₁₋₃ aliphatic), or an optionally substituted C₁₋₃ aliphatic. In some embodiments, each instance of R^{Z3C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{Z3C} is independently fluorine, chlorine, —OH, —OCH₃, —OCF₃, —CH₃, —CHF₂, or —CF₃. In some embodiments, each instance of R^{Z3C} is independently fluorine or —OH.

In some embodiments, each instance of R^{Z3C} is independently selected from the groups depicted in the compounds in Table 1.

As defined generally above, each instance of R^{L1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{L1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{L1C} is oxo. In some embodiments, R^{L1C} is deuterium. In some embodiments, each instance of R^{L1C} is independently halogen. In some embodiments, R^{L1C} is —CN. In some embodiments, R^{L1C} is —NO₂. In some embodiments, R^{L1C} is —OR. In some embodiments, R^{L1C} is —SR. In some embodiments, R^{L1C} is —NR₂. In some embodiments, R^{L1C} is —S(O)₂R. In some embodiments, R^{L1C} is —S(O)₂NR₂. In some embodiments, R^{L1C} is —S(O)₂F. In some embodiments, R^{L1C} is —S(O)R. In some embodiments, R^{L1C} is —S(O)NR₂. In some embodiments, R^{L1C} is —S(O)(NR)R. In some embodiments, R^{L1C} is —C(O)R. In some embodiments, R^{L1C} is —C(O)OR. In some embodiments, R^{L1C} is —C(O)NR₂. In some embodiments, R^{L1C} is —C(O)N(R)OR. In some embodiments, R^{L1C} is —OC(O)R. In some embodiments, R^{L1C} is —OC(O)NR₂. In some embodiments, R^{L1C} is —N(R)C(O)OR. In some embodiments, R^{L1C} is —N(R)C(O)R. In some embodiments, R^{L1C} is —N(R)C(O)NR₂. In some embodiments, R^{L1C} is —N(R)C(NR)NR₂. In some embodiments, R^{L1C} is —N(R)S(O)₂NR₂. In some embodiments, R^{L1C} is —N(R)S(O)₂R. In some embodiments, R^{L1C} is —P(O)R₂. In some embodiments, R^{L1C} is —P(O)(R)OR. In some embodiments, R^{L1C} is —B(OR)₂.

In some embodiments, each instance of R^{L1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{L1C} is independently halogen, —CN, or —NO₂. In some embodiments, each instance of R^{L1C} is independently —OR, —SR, or —NR₂. In some embodiments, each instance of R^{L1C} is independently —S(O)₂R, —S(O)₂NR₂, —S(O)₂F, —S(O)R, —S(O)NR₂, or —S(O)(NR)R. In some embodiments, each instance of R^{L1C} is independently —C(O)R, —C(O)OR, —C(O)NR₂, or —C(O)N(R)OR. In some embodiments, each instance of R^{L1C} is independently —OC(O)R or —OC(O)NR₂. In some embodiments, each instance of R^{L1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{L1C} is independently —P(O)R₂ or —P(O)(R)OR.

In some embodiments, each instance of R^{L1C} is independently —OR, —OC(O)R, or —OC(O)NR₂. In some embodiments, each instance of R^{L1C} is independently —SR, —S(O)₂R, —S(O)₂NR₂, —S(O)₂F, —S(O)R, —S(O)NR₂, or —S(O)(NR)R. In some embodiments, each instance of R^{L1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{L1C} is independently —S(O)₂R, —S(O)₂NR₂, or —S(O)₂F. In some embodiments, each instance of R^{L1C} is independently —S(O)R, —S(O)NR₂, or —S(O)(NR)R. In some embodiments, each instance of R^{L1C} is independently —SR, —S(O)₂R, or —S(O)R. In some embodiments, each instance of R^{L1C} is independently —S(O)₂NR₂, —S(O)NR₂, or —S(O)(NR)R. In some embodiments, each instance of R^{L1C} is independently —S(O)₂NR₂ or —S(O)NR₂. In some embodiments, each instance of R^{L1C} is independently —SR, —S(O)₂R, —S(O)₂NR₂, or —S(O)R.

In some embodiments, each instance of R^{L1C} is independently —N(R)C(O)OR, —N(R)C(O)R, or —N(R)C(O)NR₂. In some embodiments, each instance of R^{L1C} is independently —N(R)S(O)₂NR₂ or —N(R)S(O)₂R. In some embodiments, each instance of R^{L1C} is independently —N(R)C(O)OR or —N(R)C(O)R. In some embodiments, each instance of R^{L1C} is independently —N(R)C(O)NR₂ or —N(R)S(O)₂NR₂. In some embodiments, each instance of R^{L1C} is independently —N(R)C(O)OR, —N(R)C(O)R, or —N(R)S(O)₂R.

In some embodiments, each instance of R^{L1C} is independently —NR₂, —N(R)C(O)OR, —N(R)C(O)R, or —N(R)C(O)NR₂. In some embodiments, each instance of R^{L1C} is independently —NR₂, —N(R)C(O)OR, or —N(R)C(O)R. In some embodiments, each instance of R^{L1C} is independently —NR₂, —N(R)C(O)OR, —N(R)C(O)R, or —N(R)S(O)₂R.

In some embodiments, each instance of R^{L1C} is independently an optionally substituted C₁₋₆ aliphatic. In some embodiments, each instance of R^{L1C} is independently an optionally substituted phenyl. In some embodiments, each instance of R^{L1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{L1C} is independently an optionally substituted 5-6 membered monocyclic heteroaryl ring having 1-4 heteroatoms independently selected from nitrogen, oxygen, and sulfur.

In some embodiments, each instance of R^{L1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{L1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{L1C} is independently an optionally substituted C₁₋₆ aliphatic or an optionally substituted phenyl. In some embodiments, each instance of R^{L1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{L1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L1C} is independently a C₁₋₆ aliphatic. In some embodiments, R^{L1C} is phenyl. In some embodiments, each instance of R^{L1C} is independently a 3-7 membered saturated or partially unsaturated monocyclic heterocyclic ring having 1-2 heteroatoms independently selected from nitrogen, oxygen, and sulfur. In some embodiments, each instance of R^{L1C} is independently a 5-6 membered monocyclic heteroaryl ring having 1-4 heteroatoms independently selected from nitrogen, oxygen, and sulfur.

In some embodiments, each instance of R^{L1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{L1C} is independently phenyl or a 5-6 membered monocyclic heteroaryl ring having 1-4 heteroatoms independently selected from nitrogen, oxygen, and

In some embodiments, each instance of R^{L1C} is independently a C₁₋₆ aliphatic or phenyl. In some embodiments, each instance of R^{L1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L1C} is independently selected from the groups depicted in the compounds in Table 1.

As defined generally above, each instance of R^{EEC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EEC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{EEC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{EEC} is independently deuterium, halogen, —CN, —OR, or —NR₂. In some embodiments, each instance of R^{EEC} is independently deuterium or halogen. In some embodiments, each instance of R^{EEC} is independently halogen.

In some embodiments, each instance of R^{EEC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EEC} is independently selected from the groups depicted in the compounds in Table 1.

As defined generally above, each instance of R^{Q3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Q3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Q3C} is oxo. In some embodiments, R^{Q3C} is deuterium. In some embodiments, each instance of R^{Q3C} is independently halogen. In some embodiments, R^{Q3C} is —CN. In some embodiments, R^{Q3C} is —NO₂. In some embodiments, R^{Q3C} is —OR. In some embodiments, R^{Q3C} is —SR. In some embodiments, R^{Q3C} is —NR₂. In some embodiments, R^{Q3C} is —S(O)₂R. In some embodiments, R^{Q3C} is —S(O)₂NR₂. In some embodiments, R^{Q3C} is —S(O)₂F. In some embodiments, R^{Q3C} is —S(O)R. In some embodiments, R^{Q3C} is —S(O)NR₂. In some embodiments, R^{Q3C} is —S(O)(NR)R. In some embodiments, R^{Q3C} is —C(O)R. In some embodiments, R^{Q3C} is —C(O)OR. In some embodiments, R^{Q3C} is —C(O)NR₂. In some embodiments, R^{Q3C} is —C(O)N(R)OR. In some embodiments, R^{Q3C} is —OC(O)R. In some embodiments, R^{Q3C} is —OC(O)NR₂. In some embodiments, R^{Q3C} is —N(R)C(O)OR. In some embodiments, R^{Q3C} is —N(R)C(O)R. In some embodiments, R^{Q3C} is —N(R)C(O)NR₂. In some embodiments, R^{Q3C} is —N(R)C(NR)NR₂. In some embodiments, R^{Q3C} is —N(R)S(O)₂NR₂. In some embodiments, R^{Q3C} is —N(R)S(O)₂R. In some embodiments, R^{Q3C} is —P(O)R₂. In some embodiments, R^{Q3C} is —P(O)(R)OR. In some embodiments, R^{Q3C} is —B(OR)₂.

In some embodiments, each instance of R^{Q3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Q3C} is independently halogen, —CN, or —NO₂. In some embodiments, each instance of R^{Q3C} is independently —OR, —SR, or —NR₂. In some embodiments, each instance of R^{Q3C} is independently —S(O)₂R, —S(O)₂NR₂, —S(O)₂F, —S(O)R, —S(O)NR₂, or —S(O)(NR)R. In some embodiments, each instance of R^{Q3C} is independently —C(O)R, —C(O)OR, —C(O)NR₂, or —C(O)N(R)OR. In some embodiments, each instance of R^{Q3C} is independently —OC(O)R or —OC(O)NR₂. In some embodiments, each instance of R^{Q3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Q3C} is independently —P(O)R₂ or —P(O)(R)OR.

In some embodiments, each instance of R^{Q3C} is independently —OR, —OC(O)R, or —OC(O)NR₂. In some embodiments, each instance of R^{Q3C} is independently —SR, —S(O)₂R, —S(O)₂NR₂, —S(O)₂F, —S(O)R, —S(O)NR₂, or —S(O)(NR)R. In some embodiments, each instance of R^{Q3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Q3C} is independently —S(O)₂R, —S(O)₂NR₂, or —S(O)₂F. In some embodiments, each instance of R^{Q3C} is independently —S(O)R, —S(O)NR₂, or —S(O)(NR)R. In some embodiments, each instance of R^{Q3C} is independently —SR, —S(O)₂R, or —S(O)R. In some embodiments, each instance of R^{Q3C} is independently —S(O)₂NR₂, —S(O)NR₂, or —S(O)(NR)R. In some embodiments, each instance of R^{Q3C} is independently —S(O)₂NR₂ or —S(O)NR₂. In some embodiments, each instance of R^{Q3C} is independently —SR, —S(O)₂R, —S(O)₂NR₂, or —S(O)R.

In some embodiments, each instance of R^{Q3C} is independently —N(R)C(O)OR, —N(R)C(O)R, or —N(R)C(O)NR₂. In some embodiments, each instance of R^{Q3C} is independently —N(R)S(O)₂NR₂ or —N(R)S(O)₂R. In some embodiments, each instance of R^{Q3C} is independently —N(R)C(O)OR or —N(R)C(O)R. In some embodiments, each instance of R^{Q3C} is independently —N(R)C(O)NR₂ or —N(R)S(O)₂NR₂. In some embodiments, each instance of R^{Q3C} is independently —N(R)C(O)OR, —N(R)C(O)R, or —N(R)S(O)₂R.

In some embodiments, each instance of R^{Q3C} is independently —NR₂, —N(R)C(O)OR, —N(R)C(O)R, or —N(R)C(O)NR₂. In some embodiments, each instance of R^{Q3C} is independently —NR₂, —N(R)C(O)OR, or —N(R)C(O)R. In some embodiments, each instance of R^{Q3C} is independently —NR₂, —N(R)C(O)OR, —N(R)C(O)R, or —N(R)S(O)₂R.

In some embodiments, each instance of R^{Q3C} is independently an optionally substituted C₁₋₆ aliphatic. In some embodiments, each instance of R^{Q3C} is independently an optionally substituted phenyl. In some embodiments, each instance of R^{Q3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Q3C} is independently an optionally substituted 5-6 membered monocyclic heteroaryl ring having 1-4 heteroatoms independently selected from nitrogen, oxygen, and sulfur.

In some embodiments, each instance of R^{Q3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Q3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Q3C} is independently an optionally substituted C₁₋₆ aliphatic or an optionally substituted phenyl. In some embodiments, each instance of R^{Q3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Q3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q3C} is independently a C₁₋₆ aliphatic. In some embodiments, R^{Q3C} is phenyl. In some embodiments, each instance of R^{Q3C} is independently a 3-7 membered saturated or partially unsaturated monocyclic heterocyclic ring having 1-2 heteroatoms independently selected from nitrogen, oxygen, and sulfur. In some embodiments, each instance of R^{Q3C} is independently a 5-6 membered monocyclic heteroaryl ring having 1-4 heteroatoms independently selected from nitrogen, oxygen, and sulfur.

In some embodiments, each instance of R^{Q3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Q3C} is independently phenyl or a 5-6 membered monocyclic heteroaryl ring having 1-4 heteroatoms independently selected from nitrogen, oxygen, and sulfur.

In some embodiments, each instance of R^{Q3C} is independently a C₁₋₆ aliphatic or phenyl. In some embodiments, each instance of R^{Q3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q3C} is independently selected from the groups depicted in the compounds in Table 1.

As defined generally above, each instance of R^{Z2Z3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2Z3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2Z3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Z2Z3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Z2Z3C} is oxo. In some embodiments, R^{Z2Z3C} is deuterium. In some embodiments, each instance of R^{Z2Z3C} is independently halogen. In some embodiments, R^{Z2Z3C} is —CN. In some embodiments, R^{Z2Z3C} is —NO₂. In some embodiments, R^{Z2Z3C} is —OR. In some embodiments, R^{Z2Z3C} is —SR. In some embodiments, R^{Z2Z3C} is —NR₂. In some embodiments, R^{Z2Z3C} is —S(O)₂R. In some embodiments, R^{Z2Z3C} is —S(O)₂NR₂. In some embodiments, R^{Z2Z3C} is —S(O)₂F. In some embodiments, R^{Z2Z3C} is —S(O)R. In some embodiments, R^{Z2Z3C} is —S(O)NR₂. In some embodiments, R^{Z2Z3C} is —S(O)(NR)R. In some embodiments, R^{Z2Z3C} is —C(O)R. In some embodiments, R^{Z2Z3C} is —C(O)OR. In some embodiments, R^{Z2Z3C} is —C(O)NR₂. In some embodiments, R^{Z2Z3C} is —C(O)N(R)OR. In some embodiments, R^{Z2Z3C} is —OC(O)R. In some embodiments, R^{Z2Z3C} is —OC(O)NR₂. In some embodiments, R^{Z2Z3C} is —N(R)C(O)OR. In some embodiments, R^{Z2Z3C} is —N(R)C(O)R. In some embodiments, R^{Z2Z3C} is —N(R)C(O)NR₂. In some embodiments, R^{Z2Z3C} is —N(R)C(NR)NR₂. In some embodiments, R^{Z2Z3C} is —N(R)S(O)₂NR₂. In some embodiments, R^{Z2Z3C} is —N(R)S(O)₂R. In some embodiments, R^{Z2Z3C} is —P(O)R₂. In some embodiments, R^{Z2Z3C} is —P(O)(R)OR. In some embodiments, R^{Z2Z3C} is —B(OR)₂.

In some embodiments, each instance of R^{Z2Z3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Z2Z3C} is independently halogen, —CN, or —NO₂. In some embodiments, each instance of R^{Z2Z3C} is independently —OR, —SR, or —NR₂. In some embodiments, each instance of R^{Z2Z3C} is independently —S(O)₂R, —S(O)₂NR₂, —S(O)₂F, —S(O)R, —S(O)NR₂, or —S(O)(NR)R. In some embodiments, each instance of R^{Z2Z3C} is independently —C(O)R, —C(O)OR, —C(O)NR₂, or —C(O)N(R)OR. In some embodiments, each instance of R^{Z2Z3C} is independently —OC(O)R or —OC(O)NR₂. In some embodiments, each instance of R^{Z2Z3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Z2Z3C} is independently —P(O)R₂ or —P(O)(R)OR.

In some embodiments, each instance of R^{Z2Z3C} is independently —OR, —OC(O)R, or —OC(O)NR₂. In some embodiments, each instance of R^{Z2Z3C} is independently —SR, —S(O)₂R, —S(O)₂NR₂, —S(O)₂F, —S(O)R, —S(O)NR₂, or —S(O)(NR)R. In some embodiments, each instance of R^{Z2Z3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Z2Z3C} is independently —S(O)₂R, —S(O)₂NR₂, or —S(O)₂F. In some embodiments, each instance of R^{Z2Z3C} is independently —S(O)R, —S(O)NR₂, or —S(O)(NR)R. In some embodiments, each instance of R^{Z2Z3C} is independently —SR, —S(O)₂R, or —S(O)R. In some embodiments, each instance of R^{Z2Z3C} is independently —S(O)₂NR₂, —S(O)NR₂, or —S(O)(NR)R. In some embodiments, each instance of R^{Z2Z3C} is independently —S(O)₂NR₂ or —S(O)NR₂. In some embodiments, each instance of R^{Z2Z3C} is independently —SR, —S(O)₂R, —S(O)₂NR₂, or —S(O)R.

In some embodiments, each instance of R^{Z2Z3C} is independently —N(R)C(O)OR, —N(R)C(O)R, or —N(R)C(O)NR₂. In some embodiments, each instance of R^{Z2Z3C} is independently —N(R)S(O)₂NR₂ or —N(R)S(O)₂R. In some embodiments, each instance of R^{Z2Z3C} is independently —N(R)C(O)OR or —N(R)C(O)R. In some embodiments, each instance of R^{Z2Z3C} is independently —N(R)C(O)NR₂ or —N(R)S(O)₂NR₂. In some embodiments, each instance of R^{Z2Z3C} is independently —N(R)C(O)OR, —N(R)C(O)R, or —N(R)S(O)₂R.

In some embodiments, each instance of R^{Z2Z3C} is independently —NR₂, —N(R)C(O)OR, —N(R)C(O)R, or —N(R)C(O)NR₂. In some embodiments, each instance of R^{Z2Z3C} is independently —NR₂, —N(R)C(O)OR, or —N(R)C(O)R. In some embodiments, each instance of R^{Z2Z3C} is independently —NR₂, —N(R)C(O)OR, —N(R)C(O)R, or —N(R)S(O)₂R.

In some embodiments, each instance of R^{Z2Z3C} is independently an optionally substituted C₁₋₆ aliphatic. In some embodiments, each instance of R^{Z2Z3C} is independently an optionally substituted phenyl. In some embodiments, each instance of R^{Z2Z3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Z2Z3C} is independently an optionally substituted 5-6 membered monocyclic heteroaryl ring having 1-4 heteroatoms independently selected from nitrogen, oxygen, and sulfur.

In some embodiments, each instance of R^{Z2Z3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Z2Z3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Z2Z3C} is independently an optionally substituted C₁₋₆ aliphatic or an optionally substituted phenyl. In some embodiments, each instance of R^{Z2Z3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Z2Z3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Z2Z3C} is independently a C₁₋₆ aliphatic. In some embodiments, R^{Z2Z3C} is phenyl. In some embodiments, each instance of R^{Z2Z3C} is independently a 3-7 membered saturated or partially unsaturated monocyclic heterocyclic ring having 1-2 heteroatoms independently selected from nitrogen, oxygen, and sulfur. In some embodiments, each instance of R^{Z2Z3C} is independently a 5-6 membered monocyclic heteroaryl ring having 1-4 heteroatoms independently selected from nitrogen, oxygen, and sulfur.

In some embodiments, each instance of R^{Z2Z3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Z2Z3C} is independently phenyl or a 5-6 membered monocyclic heteroaryl ring having 1-4 heteroatoms independently selected from nitrogen, oxygen, and sulfur.

In some embodiments, each instance of R^{Z2Z3C} is independently a C₁₋₆ aliphatic or phenyl. In some embodiments, each instance of R^{Z2Z3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z2Z3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Z2Z3C} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n¹ is 0, 1, 2, 3, 4, or 5. In some embodiments, n¹ is 0. In some embodiments, n¹ is 1. In some embodiments, n¹ is 2. In some embodiments, n¹ is 3. In some embodiments, n¹ is 4. In some embodiments, n¹ is 5. In some embodiments, n¹ is 0 or 1. In some embodiments, n¹ is 0, 1, or 2. In some embodiments, n¹ is 0, 1, 2, or 3. In some embodiments, n¹ is 0, 1, 2, 3, or 4. In some embodiments, n¹ is 1 or 2. In some embodiments, n¹ is 1, 2, or 3. In some embodiments, n¹ is 1, 2, 3, or 4. In some embodiments, n¹ is 1, 2, 3, 4, or 5. In some embodiments, n¹ is 2 or 3. In some embodiments, n¹ is 2, 3, or 4. In some embodiments, n¹ is 2, 3, 4, or 5. In some embodiments, n¹ is 3 or 4. In some embodiments, n¹ is 3, 4, or 5. In some embodiments, n¹ is 4 or 5. In some embodiments, n¹ is selected from the values represented in the compounds in Table 1.

As defined generally above, p¹ is 0, 1, 2, 3, 4, or 5. In some embodiments, p¹ is 0. In some embodiments, p¹ is 1. In some embodiments, p¹ is 2. In some embodiments, p¹ is 3. In some embodiments, p¹ is 4. In some embodiments, p¹ is 5. In some embodiments, p¹ is 0 or 1. In some embodiments, p¹ is 0, 1, or 2. In some embodiments, p¹ is 0, 1, 2, or 3. In some embodiments, p¹ is 0, 1, 2, 3, or 4. In some embodiments, p¹ is 1 or 2. In some embodiments, p¹ is 1, 2, or 3. In some embodiments, p¹ is 1, 2, 3, or 4. In some embodiments, p¹ is 1, 2, 3, 4, or 5. In some embodiments, p¹ is 2 or 3. In some embodiments, p¹ is 2, 3, or 4. In some embodiments, p¹ is 2, 3, 4, or 5. In some embodiments, p¹ is 3 or 4. In some embodiments, p¹ is 3, 4, or 5. In some embodiments, p¹ is 4 or 5. In some embodiments, p¹ is selected from the values represented in the compounds in Table 1.

As defined generally above, q¹ is 0, 1, 2, 3, 4, or 5. In some embodiments, q¹ is 0. In some embodiments, q¹ is 1. In some embodiments, q¹ is 2. In some embodiments, q¹ is 3. In some embodiments, q¹ is 4. In some embodiments, q¹ is 5. In some embodiments, q¹ is 0 or 1. In some embodiments, q¹ is 0, 1, or 2. In some embodiments, q¹ is 0, 1, 2, or 3. In some embodiments, q¹ is 0, 1, 2, 3, or 4. In some embodiments, q¹ is 1 or 2. In some embodiments, q¹ is 1, 2, or 3. In some embodiments, q¹ is 1, 2, 3, or 4. In some embodiments, q¹ is 1, 2, 3, 4, or 5. In some embodiments, q¹ is 2 or 3. In some embodiments, q¹ is 2, 3, or 4. In some embodiments, q¹ is 2, 3, 4, or 5. In some embodiments, q¹ is 3 or 4. In some embodiments, q¹ is 3, 4, or 5. In some embodiments, q¹ is 4 or 5. In some embodiments, q¹ is selected from the values represented in the compounds in Table 1.

As defined generally above, s¹ is 0, 1, 2, 3, 4, or 5. In some embodiments, s¹ is 0. In some embodiments, s¹ is 1. In some embodiments, s¹ is 2. In some embodiments, s¹ is 3. In some embodiments, s¹ is 4. In some embodiments, s¹ is 5. In some embodiments, s¹ is 0 or 1. In some embodiments, s¹ is 0, 1, or 2. In some embodiments, s¹ is 0, 1, 2, or 3. In some embodiments, s¹ is 0, 1, 2, 3, or 4. In some embodiments, s¹ is 1 or 2. In some embodiments, s¹ is 1, 2, or 3. In some embodiments, s¹ is 1, 2, 3, or 4. In some embodiments, s¹ is 1, 2, 3, 4, or 5. In some embodiments, s¹ is 2 or 3. In some embodiments, s¹ is 2, 3, or 4. In some embodiments, s¹ is 2, 3, 4, or 5. In some embodiments, s¹ is 3 or 4. In some embodiments, s¹ is 3, 4, or 5. In some embodiments, s¹ is 4 or 5. In some embodiments, s¹ is selected from the values represented in the compounds in Table 1.

As defined generally above, s² is 0, 1, 2, 3, 4, or 5. In some embodiments, s² is 0. In some embodiments, s² is 1. In some embodiments, s² is 2. In some embodiments, s² is 3. In some embodiments, s² is 4. In some embodiments, s² is 5. In some embodiments, s² is 0 or 1. In some embodiments, s² is 0, 1, or 2. In some embodiments, s² is 0, 1, 2, or 3. In some embodiments, s² is 0, 1, 2, 3, or 4. In some embodiments, s² is 1 or 2. In some embodiments, s² is 1, 2, or 3. In some embodiments, s² is 1, 2, 3, or 4. In some embodiments, s² is 1, 2, 3, 4, or 5. In some embodiments, s² is 2 or 3. In some embodiments, s² is 2, 3, or 4. In some embodiments, s² is 2, 3, 4, or 5. In some embodiments, s² is 3 or 4. In some embodiments, s² is 3, 4, or 5. In some embodiments, s² is 4 or 5. In some embodiments, s² is selected from the values represented in the compounds in Table 1.

As defined generally above, s³ is 0, 1, 2, 3, 4, or 5. In some embodiments, s³ is 0. In some embodiments, s³ is 1. In some embodiments, s³ is 2. In some embodiments, s³ is 3. In some embodiments, s³ is 4. In some embodiments, s³ is 5. In some embodiments, s³ is 0 or 1. In some embodiments, s³ is 0, 1, or 2. In some embodiments, s³ is 0, 1, 2, or 3. In some embodiments, s³ is 0, 1, 2, 3, or 4. In some embodiments, s³ is 1 or 2. In some embodiments, s³ is 1, 2, or 3. In some embodiments, s³ is 1, 2, 3, or 4. In some embodiments, s³ is 1, 2, 3, 4, or 5. In some embodiments, s³ is 2 or 3. In some embodiments, s³ is 2, 3, or 4. In some embodiments, s³ is 2, 3, 4, or 5. In some embodiments, s³ is 3 or 4. In some embodiments, s³ is 3, 4, or 5. In some embodiments, s³ is 4 or 5. In some embodiments, s³ is selected from the values represented in the compounds in Table 1.

As defined generally above, s⁴ is 0, 1, 2, 3, 4, or 5. In some embodiments, s⁴ is 0. In some embodiments, s⁴ is 1. In some embodiments, s⁴ is 2. In some embodiments, s⁴ is 3. In some embodiments, s⁴ is 4. In some embodiments, s⁴ is 5. In some embodiments, s⁴ is 0 or 1. In some embodiments, s⁴ is 0, 1, or 2. In some embodiments, s⁴ is 0, 1, 2, or 3. In some embodiments, s⁴ is 0, 1, 2, 3, or 4. In some embodiments, s⁴ is 1 or 2. In some embodiments, s⁴ is 1, 2, or 3. In some embodiments, s⁴ is 1, 2, 3, or 4. In some embodiments, s⁴ is 1, 2, 3, 4, or 5. In some embodiments, s⁴ is 2 or 3. In some embodiments, s⁴ is 2, 3, or 4. In some embodiments, s⁴ is 2, 3, 4, or 5. In some embodiments, s⁴ is 3 or 4. In some embodiments, s⁴ is 3, 4, or 5. In some embodiments, s⁴ is 4 or 5. In some embodiments, s⁴ is selected from the values represented in the compounds in Table 1.

As defined generally above, s⁵ is 0, 1, 2, 3, 4, or 5. In some embodiments, s⁵ is 0. In some embodiments, s⁵ is 1. In some embodiments, s⁵ is 2. In some embodiments, s⁵ is 3. In some embodiments, s⁵ is 4. In some embodiments, s⁵ is 5. In some embodiments, s⁵ is 0 or 1. In some embodiments, s⁵ is 0, 1, or 2. In some embodiments, s⁵ is 0, 1, 2, or 3. In some embodiments, s⁵ is 0, 1, 2, 3, or 4. In some embodiments, s⁵ is 1 or 2. In some embodiments, s⁵ is 1, 2, or 3. In some embodiments, s⁵ is 1, 2, 3, or 4. In some embodiments, s⁵ is 1, 2, 3, 4, or 5. In some embodiments, s⁵ is 2 or 3. In some embodiments, s⁵ is 2, 3, or 4. In some embodiments, s⁵ is 2, 3, 4, or 5. In some embodiments, s⁵ is 3 or 4. In some embodiments, s⁵ is 3, 4, or 5. In some embodiments, s⁵ is 4 or 5. In some embodiments, s⁵ is selected from the values represented in the compounds in Table 1.

As defined generally above, s⁶ is 0, 1, 2, 3, 4, or 5. In some embodiments, s⁶ is 0. In some embodiments, s⁶ is 1. In some embodiments, s⁶ is 2. In some embodiments, s⁶ is 3. In some embodiments, s⁶ is 4. In some embodiments, s⁶ is 5. In some embodiments, s⁶ is 0 or 1. In some embodiments, s⁶ is 0, 1, or 2. In some embodiments, s⁶ is 0, 1, 2, or 3. In some embodiments, s⁶ is 0, 1, 2, 3, or 4. In some embodiments, s⁶ is 1 or 2. In some embodiments, s⁶ is 1, 2, or 3. In some embodiments, s⁶ is 1, 2, 3, or 4. In some embodiments, s⁶ is 1, 2, 3, 4, or 5. In some embodiments, s⁶ is 2 or 3. In some embodiments, s⁶ is 2, 3, or 4. In some embodiments, s⁶ is 2, 3, 4, or 5. In some embodiments, s⁶ is 3 or 4. In some embodiments, s⁶ is 3, 4, or 5. In some embodiments, s⁶ is 4 or 5. In some embodiments, s⁶ is selected from the values represented in the compounds in Table 1.

As defined generally above, s⁷ is 0, 1, 2, 3, 4, or 5. In some embodiments, s⁷ is 0. In some embodiments, s⁷ is 1. In some embodiments, s⁷ is 2. In some embodiments, s⁷ is 3. In some embodiments, s⁷ is 4. In some embodiments, s⁷ is 5. In some embodiments, s⁷ is 0 or 1. In some embodiments, s⁷ is 0, 1, or 2. In some embodiments, s⁷ is 0, 1, 2, or 3. In some embodiments, s⁷ is 0, 1, 2, 3, or 4. In some embodiments, s⁷ is 1 or 2. In some embodiments, s⁷ is 1, 2, or 3. In some embodiments, s⁷ is 1, 2, 3, or 4. In some embodiments, s⁷ is 1, 2, 3, 4, or 5. In some embodiments, s⁷ is 2 or 3. In some embodiments, s⁷ is 2, 3, or 4. In some embodiments, s⁷ is 2, 3, 4, or 5. In some embodiments, s⁷ is 3 or 4. In some embodiments, s⁷ is 3, 4, or 5. In some embodiments, s⁷ is 4 or 5. In some embodiments, s⁷ is selected from the values represented in the compounds in Table 1.

As defined generally above, s⁷ is 0, 1, 2, 3, 4, or 5. In some embodiments, s⁸ is 0. In some embodiments, s⁸ is 1. In some embodiments, s⁸ is 2. In some embodiments, s⁸ is 3. In some embodiments, s⁸ is 4. In some embodiments, s⁸ is 5. In some embodiments, s⁸ is 0 or 1. In some embodiments, s⁸ is 0, 1, or 2. In some embodiments, s⁸ is 0, 1, 2, or 3. In some embodiments, s⁸ is 0, 1, 2, 3, or 4. In some embodiments, s⁸ is 1 or 2. In some embodiments, S⁸ is 1, 2, or 3. In some embodiments, s⁸ is 1, 2, 3, or 4. In some embodiments, s⁸ is 1, 2, 3, 4, or 5. In some embodiments, s⁸ is 2 or 3. In some embodiments, s⁸ is 2, 3, or 4. In some embodiments, s⁸ is 2, 3, 4, or 5. In some embodiments, s⁸ is 3 or 4. In some embodiments, s⁸ is 3, 4, or 5. In some embodiments, s⁸ is 4 or 5. In some embodiments, s⁸ is selected from the values represented in the compounds in Table 1.

In some embodiments, the present disclosure provides a compound of formula I-b wherein E is —C(O)—, thereby forming a compound of formula I-b1:

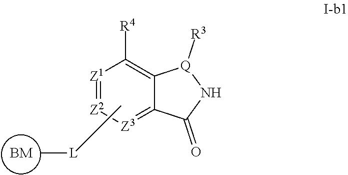

or a pharmaceutically acceptable salt thereof, wherein each of L, BM, Q, R³, R⁴, Z¹, Z², and Z³ is as defined in embodiments and classes and subclasses herein.

In some embodiments, the present disclosure provides a compound of formula I-b1 wherein Q is CH, thereby forming a compound of formula I-b2:

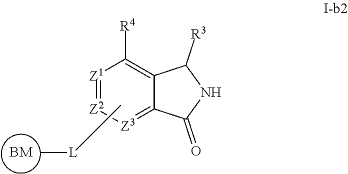

or a pharmaceutically acceptable salt thereof, wherein each of L, BM, R³, R⁴, Z¹, Z², and Z³ is as defined in embodiments and classes and subclasses herein.

In some embodiments, the present disclosure provides a compound of formula I-b1 wherein Q is N, thereby forming a compound of formula I-b3:

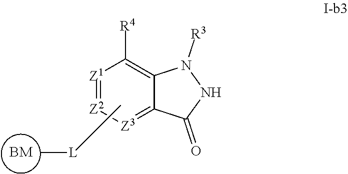

or a pharmaceutically acceptable salt thereof, wherein each of L, BM, R³, R⁴, Z¹, Z², and Z³ is as defined in embodiments and classes and subclasses herein.

In some embodiments, the present disclosure provides a compound of formula I-b1 wherein Z¹, Z², or Z³ is CH, thereby forming a compound of formula I-b4, I-b5, or I-b6:

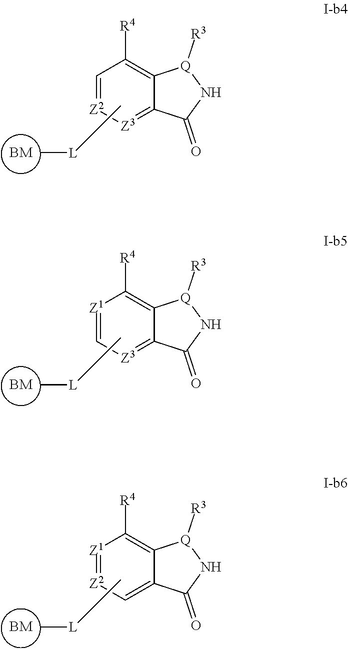

or a pharmaceutically acceptable salt thereof, wherein each of L, BM, R³, R⁴, Q, Z¹, Z², and Z³ is as defined in embodiments and classes and subclasses herein.

In some embodiments, the present disclosure provides a compound of formula I-b1 wherein Z¹, Z², or Z³ is N, thereby forming a compound of formula I-b7, I-b8, or I-b9:

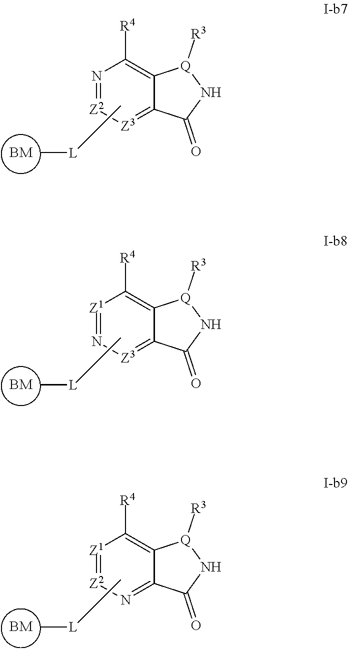

or a pharmaceutically acceptable salt thereof, wherein each of L, BM, R³, R⁴, Q, Z¹, Z², and Z³ is as defined in embodiments and classes and subclasses herein.

In some embodiments, the present disclosure provides a compound of formula I-b2 wherein X, Y, or Z is CH, thereby forming a compound of formula I-b10, I-b11, or I-b12:

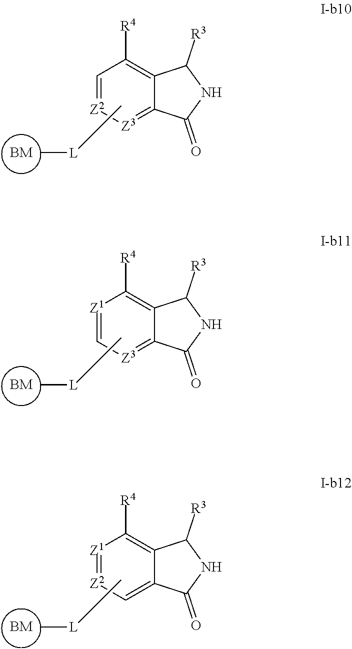

or a pharmaceutically acceptable salt thereof, wherein each of L, BM, R³, R⁴, Z¹, Z², and Z³ is as defined in embodiments and classes and subclasses herein.

In some embodiments, the present disclosure provides a compound of formula I-b wherein E is —OC(O)—, —N(R^{E})C(O)—, or —C(R^{E})₂C(O)—, thereby forming a compound of formula I-b13, I-b14, or I-b15 respectively:

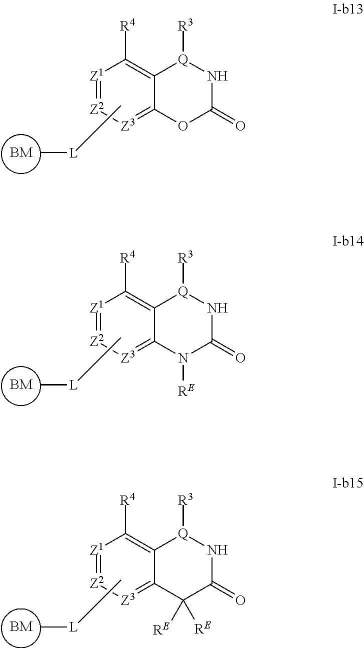

or a pharmaceutically acceptable salt thereof, wherein each of L, BM, Q, R³, R⁴, R^{E}, Z¹, Z², and Z³ is as defined in embodiments and classes and subclasses herein.

In some embodiments, the present disclosure provides a compound of formula I-b2, I-b10, I-b11, or I-b12 having the depicted stereochemistry at Q when Q is CH, thereby forming a compound of formula I-bb1, I-bb2, I-bb3, I-bb4, I-bb5, I-bb6, I-bb7, or I-bb8:

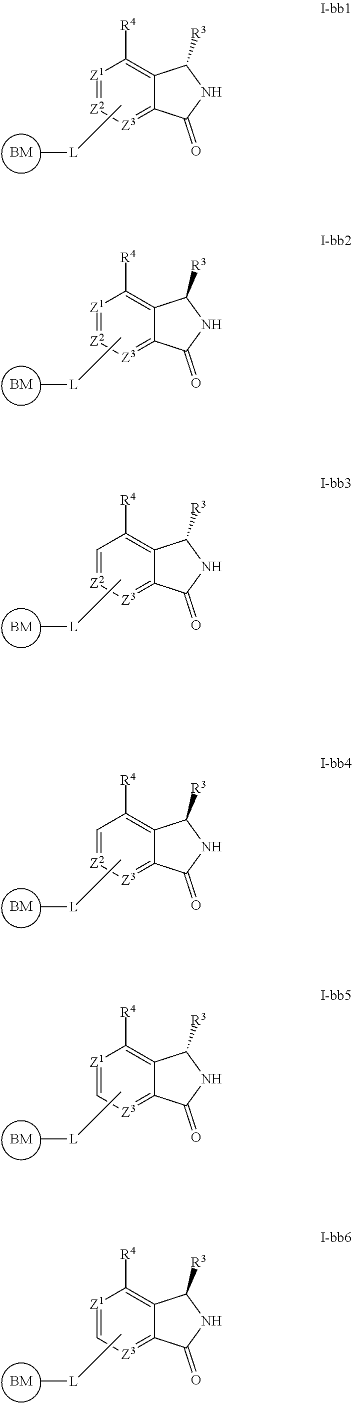

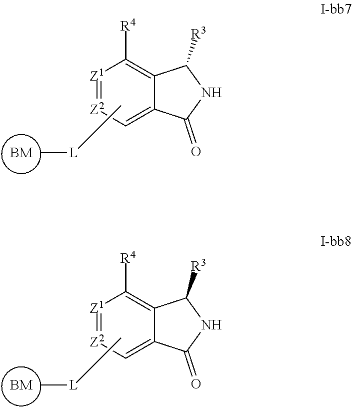

or a pharmaceutically acceptable salt thereof, wherein each of L, BM, R³, R⁴, R^{E}, Z¹, Z², and Z³ is as defined in embodiments and classes and subclasses herein.

In some embodiments, the present disclosure provides a compound of formula I-b10, I-b11, I-b12, I-bb3, I-bb5, I-bb7, I-bb4, I-bb6, or I-bb8 having the depicted point of attachment to -L-BM, thereby forming a compound of formula I-bbb1, I-bbb2, I-bbb3, I-bbb4, I-bbb5, I-bbb6, I-bbb7, I-bbb8, or I-bbb9:

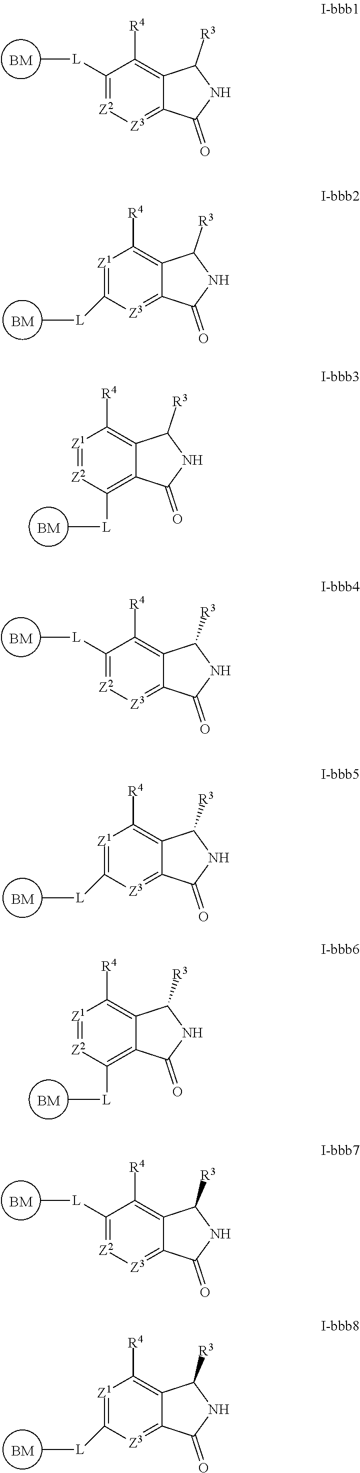

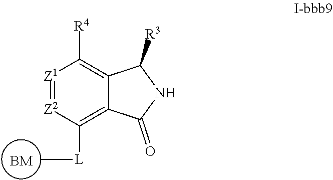

or a pharmaceutically acceptable salt thereof, wherein each of L, BM, R³, R⁴, Z¹, Z², and Z³ is as defined in embodiments and classes and subclasses herein.

In some embodiments, the present disclosure provides a compound of formula I-bb3 or I-bbb5, or a pharmaceutically acceptable salt thereof, wherein each of BM, L, R³, R⁴, Z¹, Z², and Z³ is as defined in embodiments and classes and subclasses herein.

In certain embodiments, the present disclosure provides a compound of formula I, in which PIK is a PI3K binding moiety of formula I-c0, thereby forming a compound of formula I-c:

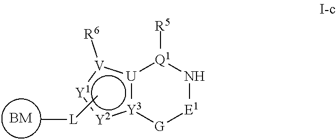

or a pharmaceutically acceptable salt thereof, wherein:
E¹ is —C(O)—, —C(R^{E1})₂—, —C(R^{E1})₂C(R^{E1})₂—, —C(S)—, —S(O)₂—, —OC(O)—, —N(R^{E1})C(O)—, —C(O)N(R^{E1})—, or —C(R^{E1})₂C(O)—;
G is CH₂, CH(R^{G}), C(R^{G})₂, or a covalent bond;
Q¹ is CH, C(R^{Q1}), or N;
Y¹ is CH, C(R^{Y1}), or N;
Y² is CH, C(R^{Y2}), N, or N(R^{Y2});
Y³ is C or N;
U is C or N;
V is C or N; provided that at least one of Y¹, Y², Y³, U, and V is N;
R⁵ is -L⁵-R^{5A};
R⁶ is -L⁶-R^{6A};
each instance of R^{E1} is independently H or -L^{E1}-R^{E1A};
each instance of R^{G} is independently -L^{G}-R^{GA};
R^{Q1} is -L^{Q1}-R^{Q1A};
R^{Y1} is -L^{Y1}-R^{Y1A};
R^{Y2} is -L^{Y2}-R^{Y2A}; or
   two instances of R^{E1} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n² instances of R^{E1EC};
   R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p² instances of R^{Q5C};
each of L⁵, L⁶, L^{E1}, L^{G}, L^{Q1}, L^{Y1}, and L^{Y2} is independently a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{5A} is R^{F} or R^{H} substituted by v¹ instances of R^{5C};
R^{6A} is R^{F} or R^{H} substituted by v² instances of R^{6C};
each instance of R^{E1A} is independently R^{F} or R^{H} substituted by v³ instances of R^{E1C};
each instance of R^{GA} is independently R^{F} or R^{H} substituted by v⁴ instances of R^{GC};
R^{Q1A} is R^{F} or R^{H} substituted by v⁵ instances of R^{Q1C};
R^{Y1A} is R^{F} or R^{H} substituted by v⁶ instances of R^{Y1C};
R^{Y2A} is R^{F} or R^{H} substituted by v⁷ instances of R^{Y2C};
R^{L2} is R^{F} or R^{H} substituted by v⁸ instances of R^{L2C};
each instance of R^{F} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{H} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{5C}, R^{6C}, R^{E1C}, R^{GC}, R^{Q1C}, R^{Y1C}, R^{Y2C}, R^{L2C}, R^{E1EC}, and R^{Q5C} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n², p², v¹, v², v³, v⁴, v⁵, v⁶, v⁷, and v⁸ is independently 0, 1, 2, 3, 4, or 5.

As defined generally above, E¹ is —C(O)—, —C(R^{E1})₂—, —C(R^{E1})₂C(R^{E1})₂—, C₃₋₆ cycloalkylene, C₃₋₆ heterocycloalkylene, —C(S)—, —S(O)₂—, —OC(O)—, —N(R^{E1})C(O)—, —C(O)N(R^{E1})—, or —C(R^{E1})₂C(O)—. In some embodiments, E¹ is —C(O)—. In some embodiments, E¹ is —OC(O)— or —N(R^{E1})C(O)—. In some embodiments, E¹ is —C(R^{E1})₂—, C₃₋₆ cycloalkylene, or C₃₋₆ heterocycloalkylene.

In some embodiments, E¹ is —C(O)—, —OC(O)—, —N(R^{E1})C(O)—, or —C(R^{E1})₂C(O)—. In some embodiments, E¹ is —OC(O)—, —N(R^{E1})C(O)—, or —C(R^{E1})₂C(O)—. In some embodiments, E¹ is —C(O)— or —N(R^{E1})C(O)—.

In some embodiments, E¹ is —C(O)—, —C(R^{E1})₂—, —C(S)—, or —S(O)₂—. In some embodiments, E¹ is —C(O)—, —C(R^{E1})₂—, or —C(S)—. In some embodiments, E¹ is —C(O)— or —C(S)—.

In some embodiments, E¹ is —C(R^{E1})₂C(R^{E1})₂—, C₃₋₆ cycloalkylene, C₃₋₆ heterocycloalkylene, —OC(O)—, —N(R^{E1})C(O)—, —C(O)N(R^{E1})—, or —C(R^{E1})₂C(O)—. In some embodiments, E¹ is C₃₋₆ cycloalkylene or C₃₋₆ heterocycloalkylene. In some embodiments, E¹ is —C(R^{E1})₂C(R^{E1})₂—, —OC(O)—, —N(R^{E1})C(O)—, —C(O)N(R^{E1})—, or —C(R^{E1})₂C(O)—. In some embodiments, E¹ is —OC(O)—, —N(R^{E1})C(O)—, —C(O)N(R^{E1})—, or —C(R^{E1})₂C(O)—. In some embodiments, E¹ is —OC(O)—, —N(R^{E1})C(O)—, or —C(O)N(R^{E1})—. In some embodiments, E¹ is —N(R^{E1})C(O)— or —C(O)N(R^{E1})—. In some embodiments, E¹ is —N(H)C(O)— or —C(O)N(H)—. In some embodiments, E¹ is —N(CH₃)C(O)— or —C(O)N(CH₃)—.

In some embodiments, E¹ is —S(O)₂—, —OC(O)—, —N(R^{E1})C(O)—, or —C(O)N(R^{E1})—. In some embodiments, E¹ is —C(O)—, —C(R^{E1})₂—, —C(R^{E1})₂C(R^{E1})₂—, C₃₋₆ cycloallylene, C₃₋₆ heterocycloalkylene, —C(S)—, or —C(R^{E1})₂C(O)—. In some embodiments, E¹ is —C(O)—, —C(R^{E1})₂—, —C(R^{E1})₂C(R^{E1})₂—, —C(S)—, or —C(R^{E1})₂C(O)—. In some embodiments, E¹ is —C(O)—, —C(S)—, or —C(R^{E1})₂C(O)—. In some embodiments, E¹ is —C(R^{E1})₂—, —C(R^{E1})₂C(R^{E1})₂—, or —C(R^{E1})₂C(O)—. In some embodiments, E¹ is —C(R^{E1})₂— or —C(R^{E1})₂C(R^{E1})₂—.

In some embodiments, E¹ is —C(R^{E1})₂—. In some embodiments, E¹ is —C(R^{E1})₂C(R^{E1})₂—. In some embodiments, E¹ is C₃₋₆ cycloalkylene. In some embodiments, E¹ is C₃₋₆ heterocycloalkylene. In some embodiments, E¹ is —C(S)—. In some embodiments, E¹ is —S(O)₂—. In some embodiments, E¹ is —OC(O)—. In some embodiments, E¹ is —N(R^{E1})C(O)—. In some embodiments, E¹ is —N(H)C(O)—. In some embodiments, E¹ is —N(CH₃)C(O)—. In some embodiments, E¹ is —C(O)N(R^{E1})—. In some embodiments, E¹ is —C(O)N(H)—. In some embodiments, E¹ is —C(O)N(CH₃)—. In some embodiments, E¹ is —C(R^{E1})₂C(O)—.

In some embodiments, E¹ is selected from the groups depicted in the compounds in Table 1.

As defined generally above, G is CH₂, CH(R^{G}), C(R^{G})₂, or a covalent bond. In some embodiments, G is CH₂, CH(R^{G}), or C(R^{G})₂. In some embodiments, G is CH₂ or CH(R^{G}). In some embodiments, G is CH(R^{G}) or C(R^{G})₂. In some embodiments, G is CH₂. In some embodiments, G is CH(R^{G}). In some embodiments, G is C(R^{G})₂. In some embodiments, G is a covalent bond. In some embodiments, G is selected from the groups depicted in the compounds in Table 1.

As defined generally above, Q¹ is CH, C(R^{Q1}), or N. In some embodiments, Q¹ is CH. In some embodiments, Q¹ is C(R^{Q1}). In some embodiments, Q¹ is N. In some embodiments, Q¹ is CH or C(R^{Q1}). In some embodiments, Q¹ is CH or N. In some embodiments, Q¹ is C(R^{Q1}) or N. In some embodiments, Q¹ is selected from the groups depicted in the compounds in Table 1.

As defined generally above, Y¹ is CH, C(R^{Y1}), or N; provided that at least one of Y¹, Y², Y³, U, and V is N. In some embodiments, Y¹ is CH. In some embodiments, Y¹ is C(R^{Y1}). In some embodiments, Y¹ is N. In some embodiments, Y¹ is CH or C(R^{Y1}). In some embodiments, Y¹ is CH or N. In some embodiments, Y¹ is C(R^{Y}) or N. In some embodiments, Y¹ is selected from the groups depicted in the compounds in Table 1.

As defined generally above, Y² is CH, C(R^{Y2}), N, or N(R^{Y2}); provided that at least one of Y¹, Y², Y³, U, and V is N. In some embodiments, Y² is CH. In some embodiments, Y² is C(R^{Y2}). In some embodiments, Y² is N. In some embodiments, Y² is N(R^{Y2}). In some embodiments, Y² is CH or C(R^{Y2}). In some embodiments, Y² is CH or N. In some embodiments, Y² is C(R^{Y2}) or N. In some embodiments, Y² is C(R^{Y2}) or N(R^{Y2}). In some embodiments, Y² is N or N(R^{Y2}). In some embodiments, Y² is selected from the groups depicted in the compounds in Table 1.

As defined generally above, Y³ is C or N; provided that at least one of Y¹, Y², Y³, U, and V is N. In some embodiments, Y³ is C. In some embodiments, Y³ is N. In some embodiments, Y³ is selected from the groups depicted in the compounds in Table 1.

As defined generally above, U is C or N; provided that at least one of Y¹, Y², Y³, U, and V is N. In some embodiments, U is C. In some embodiments, U is N. In some embodiments, U is selected from the groups depicted in the compounds in Table 1.

As defined generally above, V is C or N; provided that at least one of Y¹, Y², Y³, U, and V is N. In some embodiments, V is C. In some embodiments, V is N. In some embodiments, V is selected from the groups depicted in the compounds in Table 1.

As defined generally above, R⁵ is -L⁵-R^{5A} or R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p² instances of R^{Q5C}. In some embodiments, R⁵ is -L⁵-R^{5A}. In some embodiments, R⁵ is —R^{5A}.

In some embodiments, R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p² instances of R^{Q5C} In some embodiments, R^{Q1} and R⁵ are taken together with their intervening atoms to form a 4-8 membered saturated or partially unsaturated monocyclic ring having 0-4 heteroatoms independently selected from nitrogen, oxygen, and sulfur; wherein said ring is substituted with p² instances of R^{Q5C}. In some embodiments, R^{Q1} and R⁵ are taken together with their intervening atoms to form an 8-12 membered saturated or partially unsaturated bicyclic ring having 0-4 heteroatoms independently selected from nitrogen, oxygen, and sulfur; wherein said ring is substituted with p² instances of R^{Q5C}.

In some embodiments, R⁵ (i.e. -L⁵-R^{5A} taken together) is

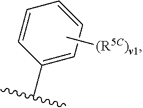

wherein R^{5C} and v¹ are as defined in the embodiments and classes and subclasses herein. In some embodiments, R⁵ (i.e. -L⁵-R^{5A} taken together) is

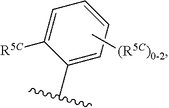

wherein R^{5C} is as defined in the embodiments and classes and subclasses herein. In some embodiments, R⁵ (i.e. -L⁵-R^{5A} taken together) is

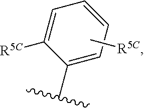

wherein R^{5C} is as defined in the embodiments and classes and subclasses herein. In some embodiments, R⁵ (i.e. -L⁵-R^{5A} taken together) is

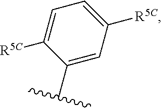

wherein R^{5C} is as defined in the embodiments and classes and subclasses herein. In some embodiments, R⁵ (i.e. -L⁵-R^{5A} taken together) is

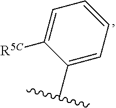

wherein R⁵ is as defined in the embodiments and classes and subclasses herein.

In some embodiments, R⁵ (i.e. -L⁵-R^{5A} taken together) is

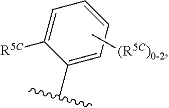

wherein each instance of R^{5C} is independently halogen, —CN, —O-(optionally substituted C₁₋₆ aliphatic), or an optionally substituted C₁₋₆ aliphatic. In some embodiments, R⁵ (i.e. -L⁵-R^{5A} taken together) is

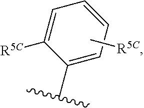

wherein each instance of R^{5C} is independently halogen or C₁₋₃ aliphatic optionally substituted with 1-3 halogen. In some embodiments, R⁵ (i.e. -L⁵-R^{5A} taken together) is

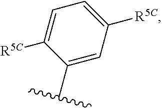

wherein each instance of R^{5C} is independently halogen or C₁₋₃ aliphatic optionally substituted with 1-3 halogen. In some embodiments, R⁵ (i.e. -L⁵-R^{5A} taken together) is

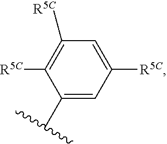

wherein each instance of R^{5C} is independently halogen or C₁₋₃ aliphatic optionally substituted with 1-3 halogen. In some embodiments, R⁵ (i.e. -L⁵-R^{5A} taken together) is

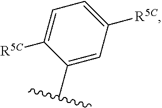

, wherein each instance of R^{5C} is independently fluorine, chlorine, —CH₃, —CHF₂, or —CF₃. In some embodiments, R⁵ (i.e. L⁵-R^{5A} taken together) is

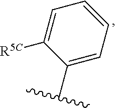

wherein R^{5C} is halogen or C₁₋₃ aliphatic optionally substituted with 1-3 halogen.

In some embodiments, R⁵ (i.e. -L⁵-R^{5A} taken together) is

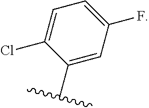 [SMILES: *c1cc(F)ccc1Cl]

In some embodiments, R⁵ (i.e. -L⁵-R^{5A} taken together) is

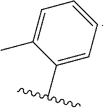 [SMILES: *c1ccccc1C]

In some embodiments, R⁵ (i.e. -L⁵-R^{5A} taken together) is

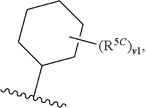

wherein R^{5C} and v¹ are as defined in the embodiments and classes and subclasses herein. In some embodiments, R⁵ (i.e. -L⁵-R^{5A} taken together) is

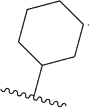 [SMILES: *C1CCCCC1]

In some embodiments, R⁵ (i.e. -L⁵-R^{5A} taken together) is

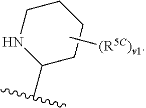

In some embodiments, R⁵ (i.e. -L⁵-R^{5A} taken together) is

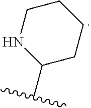 [SMILES: *C1CCCCN1]

In some embodiments, R⁵ is selected from the groups depicted in the compounds in Table 1.

As defined generally above, R⁶ is -L⁶-R^{6A}. In some embodiments, R⁶ (i.e. -L⁶-R^{6A} taken together) is —N(R)C(O)—R^{6A} or —R^{6A}, wherein R and R^{6A} are as defined in the embodiments and classes and subclasses herein. In some embodiments, R⁶ (i.e. -L⁶-R^{6A} taken together) is —N(R)C(O)—R^{6A}, wherein R and R^{6A} are as defined in the embodiments and classes and subclasses herein. In some embodiments, R⁶ (i.e. -L⁶-R^{6A} taken together) is —N(H)C(O)—R^{6A}, wherein R^{6A} is as defined in the embodiments and classes and subclasses herein. In some embodiments, R⁶ (i.e. -L⁶-R^{6A} taken together) is —N(H)C(O)—R^{6A}, wherein R^{6A} is R^{B} substituted by v instances of R^{6C}. In some embodiments, R⁶ is —R^{6A}.

In some embodiments, R⁶ is —N(H)C(O)—R^{6A}, —N(H)C(O)N(H)—R^{6A}, —C(O)N(H)—R^{6A}, —N(H)—R^{6A}, —S(O)₂CH₂—R^{6A}, —CH₂S(O)₂—R^{6A}, or —C(H)(CH₃)OH. In some embodiments, R⁶ is —N(H)C(O)—R^{6A}, —N(H)C(O)N(H)—R^{6A}, or —N(H)—R^{6A}. In some embodiments, R⁶ is —C(O)N(H)—R^{6A}, —CH₂S(O)₂—R^{6A}, or —C(H)(CH₃)OH. In some embodiments, R⁶ is —S(O)₂CH₂—R^{6A} or —CH₂S(O)₂—R^{6A}.

In some embodiments, R⁶ is —N(H)C(O)N(H)—R^{6A}. In some embodiments, R⁶ is —C(O)N(H)—R^{6A}. In some embodiments, R⁶ is —N(H)—R^{6A}. In some embodiments, R⁶ is —S(O)₂CH₂—R^{6A}. In some embodiments, R⁶ is —CH₂S(O)₂—R^{6A}. In some embodiments, R⁶ is —C(H)(CH₃)OH.

In some embodiments, R⁶ (i.e. -L⁶-R^{6A} taken together) is

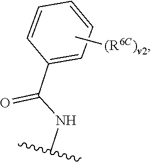

wherein R^{6C} and v² are as defined in the embodiments and classes and subclasses herein. In some embodiments, R⁶ (i.e. -L⁶-R^{6A} taken together) is

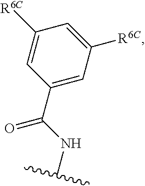

wherein R^{6C} is as defined in the embodiments and classes and subclasses herein.

In some embodiments, R⁶ (i.e. -L⁶-R^{6A} taken together) is

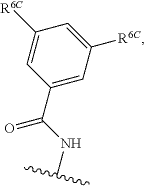

wherein each instance of R^{6C} is independently halogen, —CN, —O-(optionally substituted C₁₋₆ aliphatic), or an optionally substituted C₁₋₆ aliphatic. In some embodiments, R⁶ (i.e. -L⁶-R^{6A} taken together) is

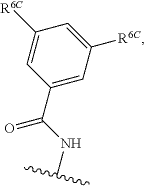

wherein each instance of R^{6C} is independently halogen or C₁₋₃ aliphatic optionally substituted with 1-3 halogen. In some embodiments, R⁶ (i.e. -L⁶-R^{6A} taken together) is

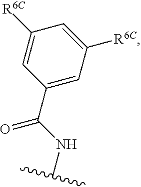

wherein each instance of R^{6C} is independently fluorine, chlorine, —CH₃, —CHF₂, or —CF₃. In some embodiments, R⁶ (i.e. -L⁶-R^{6A} taken together) is

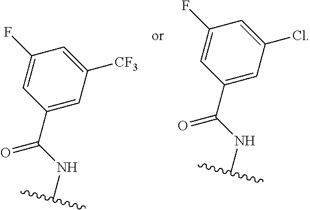 [SMILES: *NC(=O)c1cc(F)cc(C(F)(F)F)c1 ; *NC(=O)c1cc(F)cc(Cl)c1]

In some embodiments, R⁶ (i.e. -L⁶-R^{6A} taken together) is

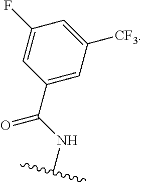 [SMILES: *NC(=O)c1cc(F)cc(C(F)(F)F)c1]

In some embodiments, R⁶ (i.e. -L⁶-R^{6A} taken together) is

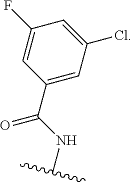 [SMILES: *NC(=O)c1cc(F)cc(Cl)c1]

In some embodiments, R⁶ (i.e. -L⁶-R^{6A} taken together) is

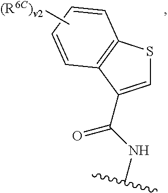

wherein R^{6C} and v² are as defined in the embodiments and classes and subclasses herein. In some embodiments, R⁶ (i.e. -L⁶-R^{6A} taken together) is

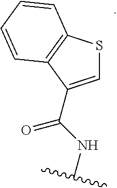 [SMILES: *NC(=O)c1csc2ccccc12]

In some embodiments, R⁶ (i.e. -L⁶-R^{6A} taken together) is

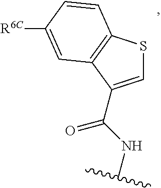

wherein R^{6C} is as defined in the embodiments and classes and subclasses herein.

In some embodiments, R⁶ (i.e. -L⁶-R^{6A} taken together) is

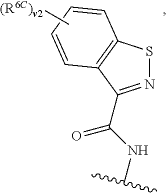

wherein R^{6C} and v² are as defined in the embodiments and classes and subclasses herein. In some embodiments, R⁶ (i.e. -L⁶-R^{6A} taken together) is

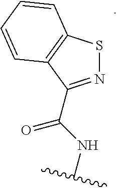 [SMILES: *NC(=O)c1nsc2ccccc12]

In some embodiments, R⁶ (i.e. -L⁶-R^{6A} taken together) is

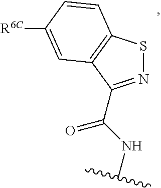

wherein R^{6C} is as defined in the embodiments and classes and subclasses herein.

In some embodiments, R⁶ (i.e. -L⁶-R^{6A} taken together) is

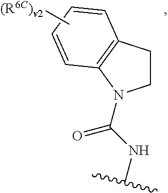

wherein R^{6C} and v² are as defined in the embodiments and classes and subclasses herein. In some embodiments, R⁶ (i.e. -L⁶-R^{6A} taken together) is

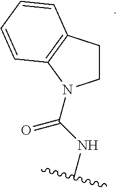 [SMILES: *NC(=O)N1CCc2ccccc21]

In some embodiments, R⁶ (i.e. -L⁶-R^{6A} taken together) is

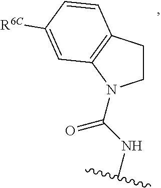

wherein R^{6C} is as defined in the embodiments and classes and subclasses herein.

In some embodiments, R⁶ (i.e. -L⁶-R^{6A} taken together) is

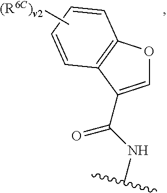

wherein R^{6C} and v² are as defined in the embodiments and classes and subclasses herein. In some embodiments, R⁶ (i.e. -L⁶-R^{6A} taken together) is

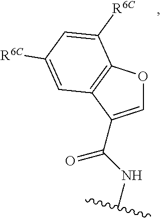

wherein R^{6C} is as defined in the embodiments and classes and subclasses herein.

In some embodiments, R⁶ (i.e. -L⁶-R^{6A} taken together) is

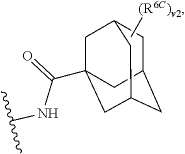

wherein R^{6C} and v² are as defined in the embodiments and classes and subclasses herein. In some embodiments, R⁶ (i.e. -L⁶-R^{6A} taken together) is

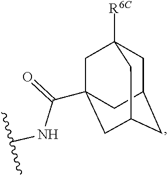

wherein R^{6C} is as defined in the embodiments and classes and subclasses herein.

In some embodiments, R⁶ (i.e. -L⁶-R^{6A} taken together) is

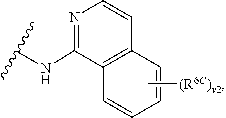

wherein R^{6C} and v² are as defined in the embodiments and classes and subclasses herein. In some embodiments, R⁶ (i.e. -L⁶-R^{6A} taken together) is

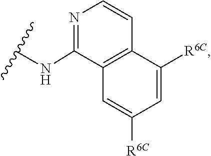

wherein R^{6C} is as defined in the embodiments and classes and subclasses herein.

In some embodiments, R⁶ (i.e. -L⁶-R^{6A} taken together) is

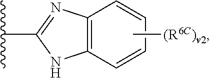

wherein R^{6C} and v² are as defined in the embodiments and classes and subclasses herein. In some embodiments, R⁶ (i.e. -L⁶-R^{6A} taken together) is

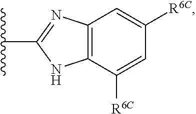

wherein R^{6C} is as defined in the embodiments and classes and subclasses herein.

In some embodiments, R⁶ is

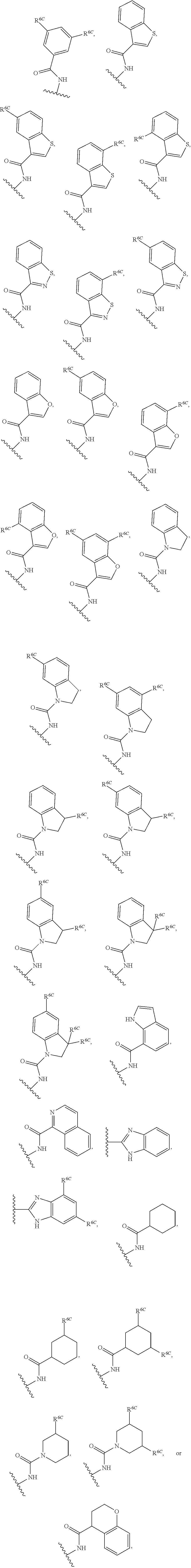

In some embodiments, R⁶ is

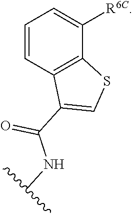

In some embodiments, R⁶ is

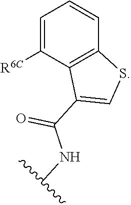

In some embodiments, R⁶ is

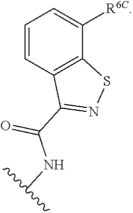

In some embodiments, R⁶ is

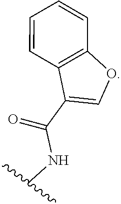 [SMILES: *NC(=O)c1coc2ccccc12]

In some embodiments, R⁶ is

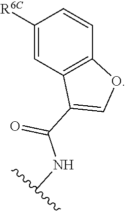

In some embodiments, R⁶ is

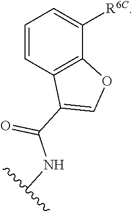

In some embodiments, R⁶ is

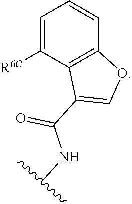

In some embodiments, R⁶ is

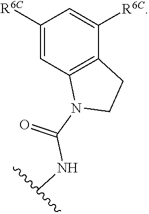

In some embodiments, R⁶ is

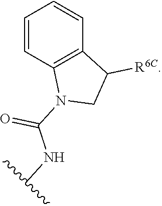

In some embodiments, R⁶ is

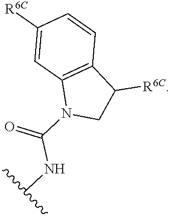

In some embodiments, R⁶ is

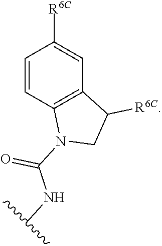

In some embodiments, R⁶ is

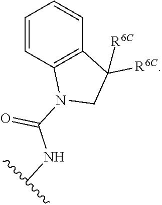

In some embodiments, R⁶ is

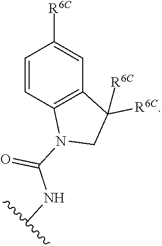

In some embodiments, R⁶ is

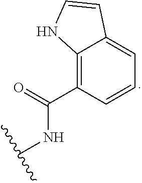 [SMILES: *NC(=O)c1cccc2cc[nH]c12]

In some embodiments, R⁶ is

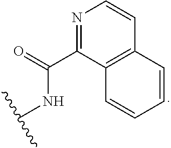 [SMILES: *NC(=O)c1nccc2ccccc12]

In some embodiments, R⁶ is

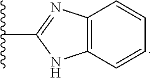 [SMILES: *c1nc2ccccc2[nH]1]

In some embodiments, R⁶ is

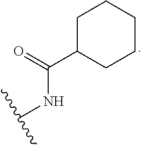 [SMILES: *NC(=O)C1CCCCC1]

In some embodiments, R⁶ is

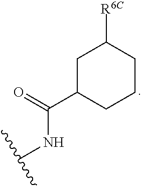

In some embodiments, R⁶ is

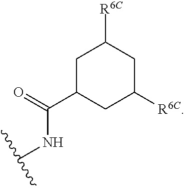

In some embodiments, R⁶ is H

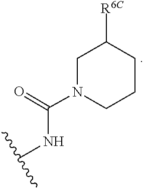

In some embodiments, R⁶ is

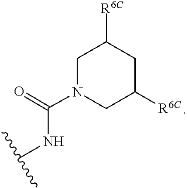

In some embodiments, R⁶ is

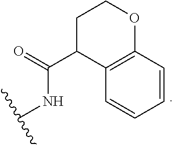 [SMILES: *NC(=O)C1CCOc2ccccc21]

In some embodiments, R⁶ is selected from the groups depicted in the compounds in Table 1.

As defined generally above, each instance of R^{E1} is independently H or -L^{E}-R^{E1A}; or two instances of R^{E1} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n² instances of R^{E1EC}.

In some embodiments, each instance of R^{E1} is independently H or -L^{E}-R^{E1A}. In some embodiments, R^{E1} is H. In some embodiments, each instance of R^{E1} is independently -L^{E}-R^{E1A}. In some embodiments, each instance of R^{E1} is independently R^{E1A}. In some embodiments, each instance of R^{E1} is independently R^{F}. In some embodiments, each instance of R^{E1} is independently R^{H} substituted by v³ instances of R^{E1C}.

In some embodiments, each instance of R^{E1} is independently H or C₁₋₆ aliphatic substituted by v³ instances of R^{E1C}. In some embodiments, each instance of R^{E1} is independently H or C₁₋₃ aliphatic substituted by v³ instances of R^{E1C}. In some embodiments, each instance of R^{E1} is independently H or C₁₋₃ aliphatic substituted by v³ instances of halogen. In some embodiments, each instance of R^{E1} is independently H or C₁₋₃ aliphatic. In some embodiments, each instance of R^{E1} is independently H, —CH₃, —CH₂F, —CHF₂—, or —CF₃. In some embodiments, each instance of R^{E1} is independently H or —CH₃.

In some embodiments, each instance of R^{E1} is independently C₁₋₆ aliphatic substituted by v³ instances of R^{E1C}. In some embodiments, each instance of R^{E1} is independently C₁₋₃ aliphatic substituted by v³ instances of R^{E1C}. In some embodiments, each instance of R^{E1} is independently C₁₋₃ aliphatic substituted by v³ instances of halogen. In some embodiments, each instance of R^{E1} is independently C₁₋₃ aliphatic. In some embodiments, each instance of R^{E1} is independently —CH₃, —CH₂F, —CHF₂—, or —CF₃. In some embodiments, R^{E1} is —CH₃.

In some embodiments, two instances of R^{E1} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n² instances of R^{E1EC}. In some embodiments, two instances of R^{E1} are taken together with their intervening atoms to form a 3-8 membered saturated or partially unsaturated monocyclic ring having 0-4 heteroatoms independently selected from nitrogen, oxygen, and sulfur; wherein said ring is substituted with n² instances of R^{E1EC}. In some embodiments, two instances of R^{E1} are taken together with their intervening atoms to form an 8-12 membered saturated or partially unsaturated bicyclic ring having 0-4 heteroatoms independently selected from nitrogen, oxygen, and sulfur; wherein said ring is substituted with n² instances of R^{E1EC}.

In some embodiments, R^{E1} is selected from the groups depicted in the compounds in Table 1.

As defined generally above, each instance of R^{G} is independently -L^{G}-R^{GA}. In some embodiments, each instance of R^{G} is independently —R^{GA}. In some embodiments, each instance of R^{G} is independently —CH₂—R^{GA}. In some embodiments, each instance of R^{G} is independently C₁₋₆ aliphatic. In some embodiments, each instance of R^{G} is independently C₁₋₃ aliphatic. In some embodiments, R^{G} is —CH₃. In some embodiments, R^{G} is selected from the groups depicted in the compounds in Table 1.

As defined generally above, R^{Q1} is -L^{Q1}-R^{Q1A} or R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p² instances of R^{Q5C}. In some embodiments, R^{Q1} is -L^{Q1}-R^{Q1A}. In some embodiments, R^{Q1} is —R^{Q1A}.

In some embodiments, R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p² instances of R^{Q5C}. In some embodiments, R^{Q1} and R⁵ are taken together with their intervening atoms to form a 4-8 membered saturated or partially unsaturated monocyclic ring having 0-4 heteroatoms independently selected from nitrogen, oxygen, and sulfur; wherein said ring is substituted with p² instances of R^{Q15C}. In some embodiments, R^{Q1} and R⁵ are taken together with their intervening atoms to form an 8-12 membered saturated or partially unsaturated bicyclic ring having 0-4 heteroatoms independently selected from nitrogen, oxygen, and sulfur; wherein said ring is substituted with p² instances of R^{Q5C}.

In some embodiments, R^{Q1} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Q1} is halogen, —CN, —OH, —O-(optionally substituted C₁₋₆ aliphatic), or an optionally substituted C₁₋₆ aliphatic. In some embodiments, R^{Q1} is halogen, —OH, or C₁₋₃ aliphatic optionally substituted with 1-3 halogen. In some embodiments, R^{Q1} is fluorine, chlorine, —OH, or —CH₃. In some embodiments, R^{Q1} is deuterium. In some embodiments, R^{Q1} is selected from the groups depicted in the compounds in Table 1.

As defined generally above, R^{Y1} is -L^{Y1}-R^{Y1A}. In some embodiments, R^{Y1} is —R^{Y1A}In some embodiments, R^{Y1} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Y1} is halogen, —CN, —OH, —O-(optionally substituted C₁₋₆ aliphatic), or an optionally substituted C₁₋₆ aliphatic. In some embodiments, R^{Y1} is halogen, —OH, —O—(C₁₋₃ aliphatic), or C₁₋₃ aliphatic, wherein each C₁₋₃ aliphatic is optionally substituted with 1-3 halogen. In some embodiments, R^{Y1} is fluorine, chlorine, —OCH₃, or —CH₃. In some embodiments, R^{Y1} is selected from the groups depicted in the compounds in Table 1.

As defined generally above, R^{Y2} is -L^{Y2}-R^{Y2A}. In some embodiments, R^{Y2} is —C(O)N(R)—R^{Y2A}, —C(O)N(R)CH₂—R^{Y2A}, or —R^{Y2A}. In some embodiments, R^{Y2} is —C(O)N(H)—R^{Y2A}, —C(O)N(H)CH₂—R^{Y2A}, or —R^{Y2A}. In some embodiments, R^{Y2} is —C(O)N(H)—R^{Y2A} or —C(O)N(H)CH₂—R^{Y2A}. In some embodiments, R^{Y2} is —C(O)N(H)—R^{Y2A}. In some embodiments, R^{Y2} is —C(O)N(H)CH₂—R^{Y2A}. In some embodiments, R^{Y2} is —R^{Y2A}.

In some embodiments, R^{Y2} is

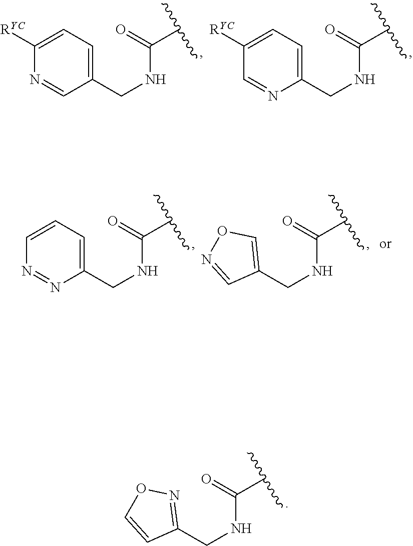

In some embodiments, R^{Y2} is

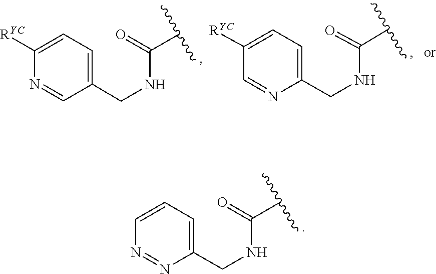

In some embodiments, R^{Y2} is

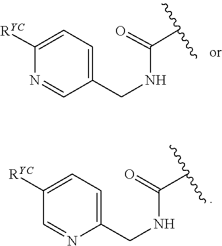

In some embodiments, R^{Y2} is

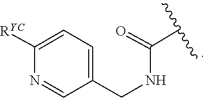

In some embodiments, R^{Y2} is

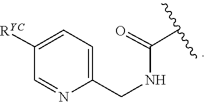

In some embodiments, R^{Y2} is

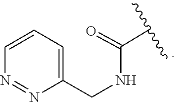 [SMILES: *C(=O)NCc1cccnn1]

In some embodiments, R^{Y2} is

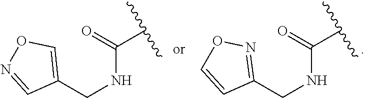 [SMILES: *C(=O)NCc1cnoc1 ; *C(=O)NCc1ccon1]

In some embodiments, R^{Y2} is

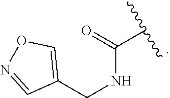 [SMILES: *C(=O)NCc1cnoc1]

In some embodiments, R^{Y2} is

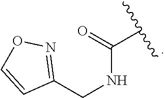 [SMILES: *C(=O)NCc1ccon1]

In some embodiments, R^{Y2} is

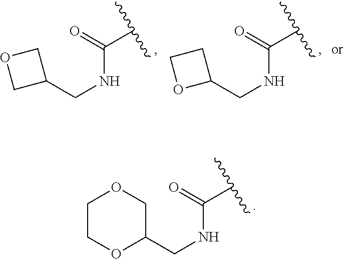

In some embodiments, R^{Y2} is

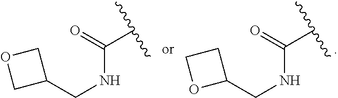 [SMILES: *C(=O)NCC1COC1 ; *C(=O)NCC1CCO1]

In some embodiments, R^{Y2} is

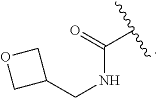 [SMILES: *C(=O)NCC1COC1]

In some embodiments, R^{Y2} is

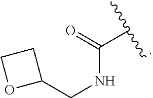 [SMILES: *C(=O)NCC1CCO1]

In some embodiments, R^{Y2} is

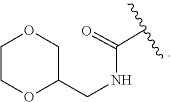 [SMILES: *C(=O)NCC1COCCO1]

In some embodiments, R^{Y2} is —C(O)N(H)—(C₁₋₆ aliphatic), wherein said C₁₋₆ aliphatic is substituted by v⁷ instances of R^{Y2C}. In some embodiments, R^{Y2} is —C(O)N(H)—(C₁₋₆ aliphatic chain), wherein said C₁₋₆ aliphatic chain is substituted by v⁷ instances of R^{Y2C}. In some embodiments, R^{Y2} is —C(O)N(H)—(C₁₋₆ aliphatic), wherein said C₁₋₆ aliphatic is optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, R^{Y2} is —C(O)N(H)—(C₁₋₆ aliphatic), wherein said C₁₋₆ aliphatic is substituted with 1 or 2 groups independently selected from —O—(C₁₋₆ aliphatic), —OH, —N(C₁₋₆ aliphatic)₂, and —CN; and said C₁₋₆ aliphatic is optionally substituted with 1, 2, or 3 halogen atoms. In some embodiments, R^{Y2} is —C(O)N(H)—(C₁₋₆ aliphatic), wherein said C₁₋₆ aliphatic is substituted with one —OH and 1, 2, or 3 halogen atoms. In some embodiments, R^{Y2} is

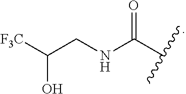 [SMILES: *C(=O)NCC(O)C(F)(F)F]

In some embodiments, R^{Y2} is —C(O)N(H)—(C₁₋₆ aliphatic), wherein said C₁₋₆ aliphatic is substituted with one —CN. In some embodiments, R^{Y2} is —C(O)N(H)—(CH₂)₂CN. In some embodiments, R^{Y2} is —C(O)N(H)—(C₁₋₆ aliphatic), wherein said C₁₋₆ aliphatic is substituted with 1, 2, or 3 halogen atoms. In some embodiments, R^{Y2} is —C(O)N(H)—CH₂CHF₂. In some embodiments, R^{Y2} is —C(O)N(H)—(C₁₋₆ aliphatic), wherein said C₁₋₆ aliphatic is substituted with 1, 2, or 3 deuterium atoms. In some embodiments, R^{Y2} is —C(O)N(H)—(C₁₋₆ aliphatic). In some embodiments, R^{Y2} is —C(O)N(H)—(C₁₋₄ allyl). In some embodiments, R^{Y2} is —C(O)N(H)CH₃. In some embodiments, R^{Y2} is —C(O)N(H)CD₃.

In some embodiments, R^{Y2} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R^{Y2} is halogen, —CN, —S(O)₂R, —S(O)₂NR₂, —S(O)₂F, —S(O)R, —S(O)NR₂, —S(O)(NR)R, —C(O)R, —C(O)OR, —C(O)NR₂, or —C(O)N(R)OR. In some embodiments, R^{Y2} is halogen or —CN. In some embodiments, R^{Y2} is selected from the groups depicted in the compounds in Table 1.

As defined generally above, L⁵ is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁵ is a covalent bond. In some embodiments, L⁵ is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁵ is a C₁₋₄ bivalent saturated or unsaturated, straight or branched hydrocarbon chain.

In some embodiments, L⁵ is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁵ is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L⁵ is a C₁₋₂ bivalent saturated or unsaturated hydrocarbon chain. In some embodiments, L⁵ is selected from the groups depicted in the compounds in Table 1.

As defined generally above, L⁶ is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁶ is a covalent bond. In some embodiments, L⁶ is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁶ is a C₁₋₄ bivalent saturated or unsaturated, straight or branched hydrocarbon chain.

In some embodiments, L⁶ is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁶ is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L⁶ is a C₁₋₂ bivalent saturated or unsaturated hydrocarbon chain.

In some embodiments, L⁶ is —N(R)C(O)— or —N(R)C(O)N(R)—. In some embodiments, L⁶ is —N(H)C(O)— or —N(H)C(O)N(H)—. In some embodiments, L⁶ is —N(R)C(O)—. In some embodiments, L⁶ is —N(H)C(O)—. In some embodiments, L⁶ is —N(R)C(O)N(R)—. In some embodiments, L⁶ is —N(H)C(O)N(H)—. In some embodiments, L⁶ is —N(R)—. In some embodiments, L⁶ is —N(H)—. In some embodiments, L⁶ is a covalent bond. In some embodiments, L⁶ is selected from the groups depicted in the compounds in Table 1.

As defined generally above, L^{E1} is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E1} is a covalent bond. In some embodiments, L^{E1} is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E1} is a bivalent saturated or unsaturated, straight or branched hydrocarbon chain.

In some embodiments, L^{E1} is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E1} is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L^{E1} is a C₁₋₂ bivalent saturated or unsaturated hydrocarbon chain. In some embodiments, L^{E1} is selected from the groups depicted in the compounds in Table 1.

As defined generally above, L^{G} is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, LG is a covalent bond. In some embodiments, L^{G} is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G} is a C₁₋₄ bivalent saturated or unsaturated, straight or branched hydrocarbon chain.

In some embodiments, L^{G} is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G} is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L^{G} is a C₁₋₂ bivalent saturated or unsaturated hydrocarbon chain. In some embodiments, L^{G} is a —CH₂—. In some embodiments, L^{G} is selected from the groups depicted in the compounds in Table 1.

As defined generally above, L^{Q1} is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q1} is a covalent bond. In some embodiments, L^{Q1} is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q1} is a C₁₋₄ bivalent saturated or unsaturated, straight or branched hydrocarbon chain.

In some embodiments, L^{Q1} is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q1} is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L^{Q1} is a C₁₋₂ bivalent saturated or unsaturated hydrocarbon chain. In some embodiments, L^{Q1} is selected from the groups depicted in the compounds in Table 1.

As defined generally above, L^{Y1} is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y1} is a covalent bond. In some embodiments, L^{Y1} is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y1} is a C₁₋₄ bivalent saturated or unsaturated, straight or branched hydrocarbon chain.

In some embodiments, L^{Y1} is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y1} is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L^{Y1} is a C₁₋₂ bivalent saturated or unsaturated hydrocarbon chain. In some embodiments, L^{Y1} is selected from the groups depicted in the compounds in Table 1.

As defined generally above, L^{Y2} is a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y2} is a covalent bond. In some embodiments, L^{Y2} is a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y2} is a C₁₋₄ bivalent saturated or unsaturated, straight or branched hydrocarbon chain.

In some embodiments, L^{Y2} is a C₁₋₂ bivalent saturated or unsaturated hydrocarbon chain wherein one or two methylene units of the chain are optionally and independently replaced by —CH(R^{L2})—, —C(R^{L2})₂—, C₃₋₆ cycloallylene, C₃₋₆ heterocycloallylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y2} is a C₁₋₂ bivalent saturated or unsaturated hydrocarbon chain wherein one or two methylene units of the chain are optionally and independently replaced by —CH(R^{L2})—, —C(R^{L2})₂—, —N(R)—, —N(R)C(O)—, —C(O)N(R)—, —N(R)S(O)₂—, —S(O)₂N(R)—, or —O—. In some embodiments, L^{Y2} is a C₁₋₂ bivalent saturated or unsaturated hydrocarbon chain.

In some embodiments, L^{Y2} is —C(O)N(R)—, —C(O)N(R)CH₂—, or a covalent bond. In some embodiments, L^{Y2} is —C(O)N(H)—, —C(O)N(H)CH₂—, or a covalent bond. In some embodiments, L^{Y2} is —C(O)N(H)— or —C(O)N(H)CH₂—. In some embodiments, L^{Y2} is —C(O)N(H)—. In some embodiments, L^{Y2} is —C(O)N(H)CH₂—. In some embodiments, L^{Y2} is selected from the groups depicted in the compounds in Table 1.

As defined generally above, R^{5A} is R^{F} or R^{H} substituted by v instances of R^{5C}. In some embodiments, R^{5A} is R^{F}. In some embodiments, R^{5A} is R^{H} substituted by v instances of R^{5C}.

In some embodiments, R^{5A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{5A} is substituted by v¹ instances of R^{5C}.

In some embodiments, R^{5A} is phenyl substituted by v¹ instances of R^{5C}. In some embodiments, R^{5A} is an 8-10 membered bicyclic heteroaryl ring having 1-4 heteroatoms independently selected from nitrogen, oxygen, and sulfur, wherein R^{5A} is substituted by v¹ instances of R^{5C}. In some embodiments, R^{5A} is phenyl or an 8-10 membered bicyclic heteroaryl ring having 1-4 heteroatoms independently selected from nitrogen, oxygen, and sulfur, wherein R^{5A} is substituted by v¹ instances of R^{5C}.

In some embodiments, R^{5A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{5A} is substituted by v¹ instances of R^{5C}.

In some embodiments, R^{5A} is phenyl substituted by v¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{5A} is an 8-10 membered bicyclic heteroaryl ring having 1-4 heteroatoms independently selected from nitrogen, oxygen, and sulfur, wherein R^{5A} is substituted by v¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{5A} is phenyl or an 8-10 membered bicyclic heteroaryl ring having 1-4 heteroatoms independently selected from nitrogen, oxygen, and sulfur, wherein R^{5A} is substituted by v¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{5A} is phenyl substituted by 1-3 instances of R^{5C}. In some embodiments, R^{5A} is phenyl substituted by 2 instances of R^{5C}. In some embodiments, R^{5A} is phenyl substituted by 1 instance of R^{5C}.

In some embodiments, R^{5A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{5A} is phenyl substituted by 1-3 instances of a group independently selected from halogen and C₁₋₃ aliphatic optionally substituted with 1-3 halogen. In some embodiments, R^{5A} is phenyl substituted by 1-3 instances of a group independently selected from fluorine, chlorine, —CH₃, —CHF₂, and —CF₃.

In some embodiments, R^{5A} is phenyl substituted by 2 instances of a group independently selected from halogen, —CN, —O-(optionally substituted C₁₋₆ aliphatic), and an optionally substituted C₁₋₆ aliphatic. In some embodiments, R^{5A} is phenyl substituted by 2 instances of a group independently selected from halogen and C₁₋₃ aliphatic optionally substituted with 1-3 halogen. In some embodiments, R^{5A} is phenyl substituted by 2 instances of a group independently selected from fluorine, chlorine, —CH₃, —CHF₂, and —CF₃.

In some embodiments, R^{5A} is phenyl substituted by one group selected from halogen, —CN, —O-(optionally substituted C₁₋₆ aliphatic), and an optionally substituted C₁₋₆ aliphatic. In some embodiments, R^{5A} is phenyl substituted by one halogen or C₁₋₃ aliphatic group optionally substituted with 1-3 halogen. In some embodiments, R^{5A} is phenyl substituted by one fluorine, chlorine, —CH₃, —CHF₂, or —CF₃.

In some embodiments, R^{5A} is

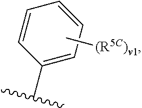

wherein R^{5C} and v¹ are as defined in the embodiments and classes and subclasses herein. In some embodiments, R^{5A} is

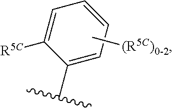

wherein R^{5C} is as defined in the embodiments and classes and subclasses herein. In some embodiments, R^{5A} is

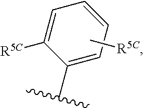

wherein R^{5C} is as defined in the embodiments and classes and subclasses herein. In some embodiments, R^{5A} is

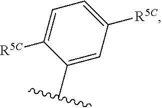

wherein R^{5C} is as defined in the embodiments and classes and subclasses herein. In some embodiments, R^{5A} is

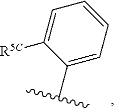

wherein R^{5C} is as defined in the embodiments and classes and subclasses herein.

In some embodiments, R^{5A} is

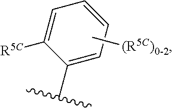

wherein each instance of R^{5C} is independently halogen, —CN, —O-(optionally substituted C₁₋₆ aliphatic), or an optionally substituted C₁₋₆ aliphatic. In some embodiments, R^{5A} is

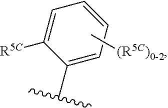

wherein each instance of R^{5C} is independently halogen or Cia aliphatic optionally substituted with 1-3 halogen. In some embodiments, R^{5A} is

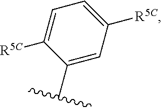

wherein each instance of R^{5C} is independently halogen or C₁₋₃ aliphatic optionally substituted with 1-3 halogen. In some embodiments, R^{5A} is

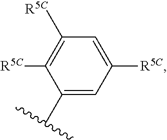

wherein each instance of R^{5C} is independently halogen or C₁₋₃ aliphatic optionally substituted with 1-3 halogen. In some embodiments, R^{5A} is

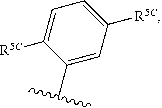

wherein each instance of R^{5C} is independently fluorine, chlorine, —CH₃, —CHF₂, or —CF₃. In some embodiments, R^{5A} is

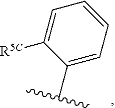

wherein R^{5C} is halogen or C₁₋₃ aliphatic optionally substituted with 1-3 halogen.

In some embodiments, R^{5A} is

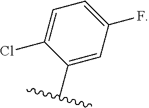 [SMILES: *c1cc(F)ccc1Cl]

In some embodiments, R^{5A} is

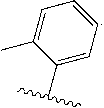 [SMILES: *c1ccccc1C]

In some embodiments, R^{5A} is

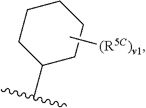

wherein R^{5C} and v¹ are as defined in the embodiments and classes and subclasses herein. In some embodiments, R^{5A} is

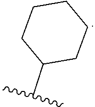 [SMILES: *C1CCCCC1]

In some embodiments, R^{5A} is

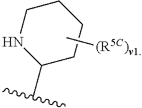

In some embodiments, R^{5A} is

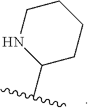 [SMILES: *C1CCCCN1]

In some embodiments, R^{5A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{5A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{5A} is oxo. In some embodiments, R^{5A} is halogen. In some embodiments, R^{5A} is —CN. In some embodiments, R^{5A} is —NO₂. In some embodiments, R^{5A} is —OR. In some embodiments, R^{5A} is —SR. In some embodiments, R^{5A} is —NR₂. In some embodiments, R^{5A} is —S(O)₂R. In some embodiments, R^{5A} is —S(O)₂NR₂. In some embodiments, R^{5A} is —S(O)₂F. In some embodiments, R^{5A} is —S(O)R. In some embodiments, R^{5A} is —S(O)NR₂. In some embodiments, R^{5A} is —S(O)(NR)R. In some embodiments, R^{5A} is —C(O)R. In some embodiments, R^{5A} is —C(O)OR. In some embodiments, R^{5A} is —C(O)NR₂. In some embodiments, R^{5A} is —C(O)N(R)OR. In some embodiments, R^{5A} is —OC(O)R. In some embodiments, R^{5A} is —OC(O)NR₂. In some embodiments, R^{5A} is —N(R)C(O)OR. In some embodiments, R^{5A} is —N(R)C(O)R. In some embodiments, R^{5A} is —N(R)C(O)NR₂. In some embodiments, R^{5A} is —N(R)C(NR)NR₂. In some embodiments, R^{5A} is —N(R)S(O)₂NR₂. In some embodiments, R^{5A} is —N(R)S(O)₂R. In some embodiments, R^{5A} is —P(O)R₂. In some embodiments, R^{5A} is —P(O)(R)OR. In some embodiments, R^{5A} is —B(OR)₂. In some embodiments, R^{5A} is deuterium.

In some embodiments, R^{5A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{5A} is halogen, —CN, or —NO₂. In some embodiments, R^{5A} is —OR, —SR, or —NR₂. In some embodiments, R^{5A} is —S(O)₂R, —S(O)₂NR₂, —S(O)₂F, —S(O)R, —S(O)NR₂, or —S(O)(NR)R. In some embodiments, R^{5A} is —C(O)R, —C(O)OR, —C(O)NR₂, or —C(O)N(R)OR. In some embodiments, R^{5A} is —OC(O)R or —OC(O)NR₂. In some embodiments, R^{5A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{5A} is —P(O)R₂ or —P(O)(R)OR.

In some embodiments, R^{5A} is —OR, —OC(O)R, or —OC(O)NR₂. In some embodiments, R^{5A} is —SR, —S(O)₂R, —S(O)₂NR₂, —S(O)₂F, —S(O)R, —S(O)NR₂, or —S(O)(NR)R. In some embodiments, R^{5A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{5A} is —S(O)₂R, —S(O)₂NR₂, or —S(O)₂F. In some embodiments, R^{5A} is —S(O)R, —S(O)NR₂, or —S(O)(NR)R. In some embodiments, R^{5A} is —SR, —S(O)₂R, or —S(O)R. In some embodiments, R^{5A} is —S(O)₂NR₂, —S(O)NR₂, or —S(O)(NR)R. In some embodiments, R^{5A} is —S(O)₂NR₂ or —S(O)NR₂. In some embodiments, R^{5A} is —SR, —S(O)₂R, —S(O)₂NR₂, or —S(O)R.

In some embodiments, R^{5A} is —N(R)C(O)OR, —N(R)C(O)R, or —N(R)C(O)NR₂. In some embodiments, R^{5A} is —N(R)S(O)₂NR₂ or —N(R)S(O)₂R. In some embodiments, R^{5A} is —N(R)C(O)OR or —N(R)C(O)R. In some embodiments, R^{5A} is —N(R)C(O)NR₂ or —N(R)S(O)₂NR₂. In some embodiments, R^{5A} is —N(R)C(O)OR, —N(R)C(O)R, or —N(R)S(O)₂R.

In some embodiments, R^{5A} is —NR₂, —N(R)C(O)OR, —N(R)C(O)R, or —N(R)C(O)NR₂. In some embodiments, R^{5A} is —NR₂, —N(R)C(O)OR, or —N(R)C(O)R. In some embodiments, R^{5A} is —NR₂, —N(R)C(O)OR, —N(R)C(O)R, or —N(R)S(O)₂R.

In some embodiments, R^{5A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is a C₁₋₆ aliphatic chain substituted by v¹ instances of R^{5C} In some embodiments, R^{5A} is phenyl substituted by v¹ instances of R^{5C}. In some embodiments, R^{5A} is naphthyl substituted by v¹ instances of R^{5C}. In some embodiments, R^{5A} is a 5-6 membered monocyclic heteroaryl ring having 1-4 heteroatoms independently selected from nitrogen, oxygen, and sulfur; wherein said ring is substituted by v¹ instances of R^{5C}. In some embodiments, R^{5A} is an 8-10 membered bicyclic heteroaryl ring having 1-4 heteroatoms independently selected from nitrogen, oxygen, and sulfur; wherein said ring is substituted by v¹ instances of R^{5C}. In some embodiments, R^{5A} is a 3-7 membered saturated or partially unsaturated monocyclic carbocyclic ring substituted by v¹ instances of R^{5C}. In some embodiments, R^{5A} is a 5-12 membered saturated or partially unsaturated bicyclic carbocyclic ring substituted by v¹ instances of R^{5C}. In some embodiments, R^{5A} is a 3-7 membered saturated or partially unsaturated monocyclic heterocyclic ring having 1-2 heteroatoms independently selected from nitrogen, oxygen, and sulfur; wherein said ring is substituted by v¹ instances of R^{5C}. In some embodiments, R^{5A} is a 7-12 membered saturated or partially unsaturated bicyclic heterocyclic ring having 1-4 heteroatoms independently selected from nitrogen, oxygen, and sulfur; wherein said ring is substituted by v¹ instances of R^{5C}.

In some embodiments, R^{5A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v¹ instances of R^{5C}. In some embodiments, R^{5A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is phenyl; naphthyl; a 3-7 membered saturated or partially unsaturated monocyclic carbocyclic ring; or a 5-12 membered saturated or partially unsaturated bicyclic carbocyclic ring; each of which is substituted by v¹ instances of R^{5C}. In some embodiments, R^{5A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v¹ instances of R^{5C}. In some embodiments, R^{5A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is phenyl or naphthyl; each of which is substituted by v¹ instances of R^{5C}. In some embodiments, R^{5A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v¹ instances of R^{5C}. In some embodiments, R^{5A} is a 3-7 membered saturated or partially unsaturated monocyclic carbocyclic ring or a 5-12 membered saturated or partially unsaturated bicyclic carbocyclic ring; each of which is substituted by v¹ instances of R^{5C}. In some embodiments, R^{5A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is phenyl or a 5-6 membered monocyclic heteroaryl ring having 1-4 heteroatoms independently selected from nitrogen, oxygen, and sulfur; each of which is substituted by v¹ instances of R^{5C}. In some embodiments, R^{5A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by v¹ instances of R^{5C}. In some embodiments, R^{5A} is naphthyl or an 8-10 membered bicyclic heteroaryl ring having 1-4 heteroatoms independently selected from nitrogen, oxygen, and sulfur; each of which is substituted by v¹ instances of R^{5C}. In some embodiments, R^{5A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is phenyl or a 3-7 membered saturated or partially unsaturated monocyclic carbocyclic ring; each of which is substituted by v¹ instances of R^{5C} In some embodiments, R^{5A} is naphthyl or a 5-12 membered saturated or partially unsaturated bicyclic carbocyclic ring; each of which is substituted by v¹ instances of R^{5C}. In some embodiments, R^{5A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v¹ instances of R^{5C}. In some embodiments, R^{5A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}.

In some embodiments, R^{5A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v¹ instances of R^{5C}. In some embodiments, R^{5A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by v¹ instances of R^{5C}. In some embodiments, R^{5A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v¹ instances of R^{5C}.

In some embodiments, R^{5A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by v¹ instances of R^{5C}. In some embodiments, R^{5A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by v¹ instances of R^{5C}. In some embodiments, R^{5A} is a C₁₋₆ aliphatic chain, phenyl, or a 3-7 membered saturated or partially unsaturated monocyclic carbocyclic ring; each of which is substituted by v¹ instances of R^{5C}.

In some embodiments, R^{5A} is selected from the groups depicted in the compounds in Table 1.

As defined generally above, R^{6A} is R^{F} or R^{H} substituted by v instances of R^{6C}. In some embodiments, R^{6A} is R^{F}. In some embodiments, R^{6A} is R^{H} substituted by v instances of R^{6C}.

In some embodiments, R^{6A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of R^{6C}.

In some embodiments, R^{6A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of R^{6C}. In some embodiments, R^{6A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of R^{6C}. In some embodiments, R^{6A} is phenyl or an 8-10 membered bicyclic heteroaryl ring having 1-4 heteroatoms independently selected from nitrogen, oxygen, and sulfur; wherein R^{6A} is substituted by v² instances of R^{6C}.

In some embodiments, R^{6A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{6A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{6A} is phenyl or an 8-10 membered bicyclic heteroaryl ring having 1-4 heteroatoms independently selected from nitrogen, oxygen, and sulfur; wherein R^{6A} is substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{6A} is phenyl substituted by v² instances of R^{6C}. In some embodiments, R^{6A} is phenyl substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{6A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{6A} is phenyl substituted by 1-3 instances of a group independently selected from halogen and C₁₋₃ aliphatic optionally substituted with 1-3 halogen. In some embodiments, R^{6A} is phenyl substituted by 1-3 instances of a group independently selected from fluorine, chlorine, —CH₃, —CHF₂, and —CF₃.

In some embodiments, R^{6A} is phenyl substituted by 2 instances of a group independently selected from halogen, —CN, —O-(optionally substituted C₁₋₆ aliphatic), and an optionally substituted C₁₋₆ aliphatic. In some embodiments, R^{6A} is phenyl substituted by 2 instances of a group independently selected from halogen and C₁₋₃ aliphatic optionally substituted with 1-3 halogen. In some embodiments, R^{6A} is phenyl substituted by 2 instances of a group independently selected from fluorine, chlorine, —CH₃, —CHF₂, and —CF₃.

In some embodiments, R^{6A} is an 8-10 membered bicyclic heteroaryl ring having 1-4 heteroatoms independently selected from nitrogen, oxygen, and sulfur; wherein R^{6A} is substituted by v² instances of R^{6C}. In some embodiments, R^{6A} is an 8-10 membered bicyclic heteroaryl ring having 1-4 heteroatoms independently selected from nitrogen, oxygen, and sulfur; wherein R^{6A} is substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{6A} is an 8-10 membered bicyclic heteroaryl ring having 1-2 heteroatoms independently selected from nitrogen, oxygen, and sulfur; wherein R^{6A} is substituted by v² instances of R^{6C}. In some embodiments, R^{6A} is an 8-10 membered bicyclic heteroaryl ring having 1-2 heteroatoms independently selected from nitrogen, oxygen, and sulfur; wherein R^{6A} is substituted by v² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{6A} is an 8-10 membered bicyclic heteroaryl ring having 1-2 heteroatoms independently selected from nitrogen, oxygen, and sulfur; wherein R^{6A} is substituted by 0-2 instances of a group independently selected from halogen, —CN, —O-(optionally substituted C₁₋₆ aliphatic), and an optionally substituted C₁₋₆ aliphatic. In some embodiments, R^{6A} is an 8-10 membered bicyclic heteroaryl ring having 1-2 heteroatoms independently selected from nitrogen, oxygen, and sulfur; wherein R^{6A} is substituted by 0-2 instances of a group independently selected from halogen and C₁₋₃ aliphatic optionally substituted with 1-3 halogen. In some embodiments, R^{6A} is an 8-10 membered bicyclic heteroaryl ring having 1-2 heteroatoms independently selected from nitrogen, oxygen, and sulfur; wherein R^{6A} is substituted by 0-2 instances of a group independently selected from fluorine, chlorine, —CH₃, —CHF₂, and —CF₃.

In some embodiments, R^{6A} is:

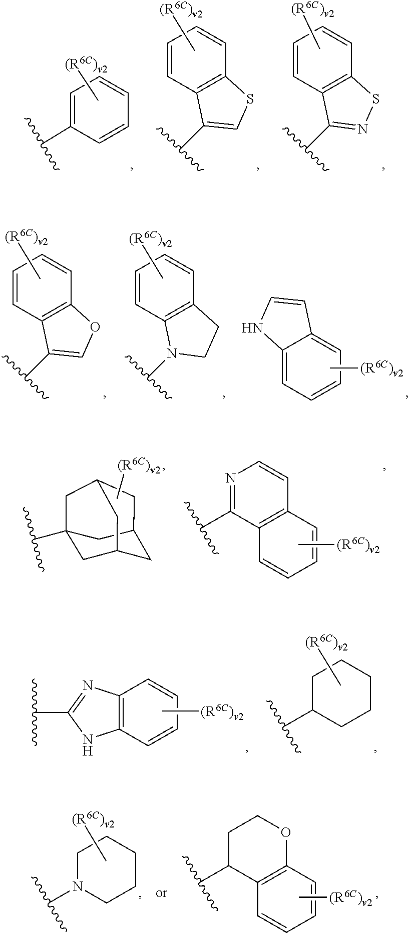

wherein R^{6C} and v² are as defined in the embodiments and classes and subclasses herein. In some embodiments, R^{6A} is

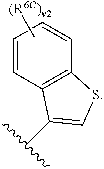

In some embodiments, R^{6A} is

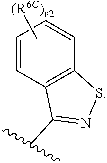

In some embodiments, R^{6A} is

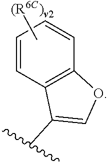

In some embodiments, R^{6A} is

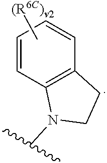

In some embodiments, R^{6A} is

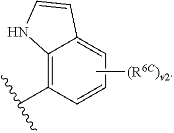

In some embodiments, R^{6A} is

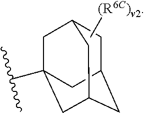

In some embodiments, R^{6A} is

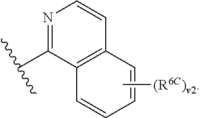

In some embodiments, R^{6A} is

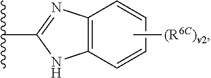

In some embodiments, R^{6A} is

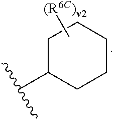

In some embodiments, R^{6A} is

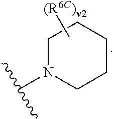

In some embodiments, R^{6A} is

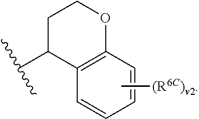

In some embodiments, R^{6A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{6A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{6A} is oxo. In some embodiments, R^{6A} is halogen. In some embodiments, R^{6A} is —CN. In some embodiments, R^{6A} is —NO₂. In some embodiments, R^{6A} is —OR. In some embodiments, R^{6A} is —SR. In some embodiments, R^{6A} is —NR₂. In some embodiments, R^{6A} is —S(O)₂R. In some embodiments, R^{6A} is —S(O)₂NR₂. In some embodiments, R^{6A} is —S(O)₂F. In some embodiments, R^{6A} is —S(O)R. In some embodiments, R^{6A} is —S(O)NR₂. In some embodiments, R^{6A} is —S(O)(NR)R. In some embodiments, R^{6A} is —C(O)R. In some embodiments, R^{6A} is —C(O)OR. In some embodiments, R^{6A} is —C(O)NR₂. In some embodiments, R^{6A} is —C(O)N(R)OR. In some embodiments, R^{6A} is —OC(O)R. In some embodiments, R^{6A} is —OC(O)NR₂. In some embodiments, R^{6A} is —N(R)C(O)OR. In some embodiments, R^{6A} is —N(R)C(O)R. In some embodiments, R^{6A} is —N(R)C(O)NR₂. In some embodiments, R^{6A} is —N(R)C(NR)NR₂. In some embodiments, R^{6A} is —N(R)S(O)₂NR₂. In some embodiments, R^{6A} is —N(R)S(O)₂R. In some embodiments, R^{6A} is —P(O)R₂. In some embodiments, R^{6A} is —P(O)(R)OR. In some embodiments, R^{6A} is —B(OR)₂. In some embodiments, R^{6A} is deuterium.

In some embodiments, R^{6A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{6A} is halogen, —CN, or —NO₂. In some embodiments, R^{6A} is —OR, —SR, or —NR₂. In some embodiments, R^{6A} is —S(O)₂R, —S(O)₂NR₂, —S(O)₂F, —S(O)R, —S(O)NR₂, or —S(O)(NR)R. In some embodiments, R^{6A} is —C(O)R, —C(O)OR, —C(O)NR₂, or —C(O)N(R)OR. In some embodiments, R^{6A} is —OC(O)R or —OC(O)NR₂. In some embodiments, R^{6A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{6A} is —P(O)R₂ or —P(O)(R)OR.

In some embodiments, R^{6A} is —OR, —OC(O)R, or —OC(O)NR₂. In some embodiments, R^{6A} is —SR, —S(O)₂R, —S(O)₂NR₂, —S(O)₂F, —S(O)R, —S(O)NR₂, or —S(O)(NR)R. In some embodiments, R^{6A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{6A} is —S(O)₂R, —S(O)₂NR₂, or —S(O)₂F. In some embodiments, R^{6A} is —S(O)R, —S(O)NR₂, or —S(O)(NR)R. In some embodiments, R^{6A} is —SR, —S(O)₂R, or —S(O)R. In some embodiments, R^{6A} is —S(O)₂NR₂, —S(O)NR₂, or —S(O)(NR)R. In some embodiments, R^{6A} is —S(O)₂NR₂ or —S(O)NR₂. In some embodiments, R^{6A} is —SR, —S(O)₂R, —S(O)₂NR₂, or —S(O)R.

In some embodiments, R^{6A} is —N(R)C(O)OR, —N(R)C(O)R, or —N(R)C(O)NR₂. In some embodiments, R^{6A} is —N(R)S(O)₂NR₂ or —N(R)S(O)₂R. In some embodiments, R^{6A} is —N(R)C(O)OR or —N(R)C(O)R. In some embodiments, R^{6A} is —N(R)C(O)NR₂ or —N(R)S(O)₂NR₂. In some embodiments, R^{6A} is —N(R)C(O)OR, —N(R)C(O)R, or —N(R)S(O)₂R.

In some embodiments, R^{6A} is —NR₂, —N(R)C(O)OR, —N(R)C(O)R, or —N(R)C(O)NR₂. In some embodiments, R^{6A} is —NR₂, —N(R)C(O)OR, or —N(R)C(O)R. In some embodiments, R^{6A} is —NR₂, —N(R)C(O)OR, —N(R)C(O)R, or —N(R)S(O)₂R.

In some embodiments, R^{6A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is a C₁₋₆ aliphatic chain substituted by v² instances of R^{6C} In some embodiments, R^{6A} is phenyl substituted by v² instances of R^{6C}. In some embodiments, R^{6A} is naphthyl substituted by v² instances of R^{6C}. In some embodiments, R^{6A} is cubanyl substituted by v² instances of R^{6C}. In some embodiments, R^{6A} is adamantyl substituted by v² instances of R^{6C}. In some embodiments, R^{6A} is a 5-6 membered monocyclic heteroaryl ring having 1-4 heteroatoms independently selected from nitrogen, oxygen, and sulfur; wherein said ring is substituted by v² instances of R^{6C}. In some embodiments, R^{6A} is an 8-10 membered bicyclic heteroaryl ring having 1-4 heteroatoms independently selected from nitrogen, oxygen, and sulfur; wherein said ring is substituted by v² instances of R^{6C}. In some embodiments, R^{6A} is a 3-7 membered saturated or partially unsaturated monocyclic carbocyclic ring substituted by v² instances of R^{6C}. In some embodiments, R^{6A} is a 5-12 membered saturated or partially unsaturated bicyclic carbocyclic ring substituted by v² instances of R^{6C}. In some embodiments, R^{6A} is a 3-7 membered saturated or partially unsaturated monocyclic heterocyclic ring having 1-2 heteroatoms independently selected from nitrogen, oxygen, and sulfur; wherein said ring is substituted by v² instances of R^{6C}. In some embodiments, R^{6A} is a 7-12 membered saturated or partially unsaturated bicyclic heterocyclic ring having 1-4 heteroatoms independently selected from nitrogen, oxygen, and sulfur; wherein said ring is substituted by v² instances of R^{6C}.

In some embodiments, R^{6A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v² instances of R^{6C}. In some embodiments, R^{6A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by v² instances of R^{6C}. In some embodiments, R^{6A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v² instances of R^{6C}. In some embodiments, R^{6A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is phenyl or naphthyl; each of which is substituted by v² instances of R^{6C}. In some embodiments, R^{6A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v² instances of R^{6C}. In some embodiments, R^{6A} is a 3-7 membered saturated or partially unsaturated monocyclic carbocyclic ring or a 5-12 membered saturated or partially unsaturated bicyclic carbocyclic ring; each of which is substituted by v² instances of R^{6C}. In some embodiments, R^{6A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is phenyl or a 5-6 membered monocyclic heteroaryl ring having 1-4 heteroatoms independently selected from nitrogen, oxygen, and sulfur; each of which is substituted by v² instances of R^{6C}. In some embodiments, R^{6A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by v² instances of R^{6C}. In some embodiments, R^{6A} is naphthyl or an 8-10 membered bicyclic heteroaryl ring having 1-4 heteroatoms independently selected from nitrogen, oxygen, and sulfur; each of which is substituted by v² instances of R^{6C}. In some embodiments, R^{6A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is phenyl or a 3-7 membered saturated or partially unsaturated monocyclic carbocyclic ring; each of which is substituted by v² instances of R^{6C}In some embodiments, R^{6A} is naphthyl; cubanyl; adamantyl; or a 5-12 membered saturated or partially unsaturated bicyclic carbocyclic ring; each of which is substituted by v² instances of R^{6C}. In some embodiments, R^{6A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v² instances of R^{6C}. In some embodiments, R^{6A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}.

In some embodiments, R^{6A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v² instances of R^{6C}. In some embodiments, R^{6A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by v² instances of R^{6C}. In some embodiments, R^{6A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v² instances of R^{6C}.

In some embodiments, R^{6A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by v² instances of R^{6C}. In some embodiments, R^{6A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by v² instances of R^{6C}. In some embodiments, R^{6A} is a C₁₋₆ aliphatic chain, phenyl, or a 3-7 membered saturated or partially unsaturated monocyclic carbocyclic ring; each of which is substituted by v² instances of R^{6C}.

In some embodiments, R^{6A} is selected from the groups depicted in the compounds in Table 1.

As defined generally above, each instance of R^{E1A} is independently R^{F} or R^{H} substituted by v³ instances of R^{E1C}. In some embodiments, each instance of R^{E1A} is independently R^{F}. In some embodiments, each instance of R^{E1A} is independently R^{H} substituted by v³ instances of R^{E1C}.

In some embodiments, each instance of R^{E1A} is independently C₁₋₆ aliphatic substituted by v³ instances of R^{E1C}. In some embodiments, each instance of R^{EA} is independently C₁₋₃ aliphatic substituted by v³ instances of R^{E1C}. In some embodiments, each instance of R^{E1A} is independently C₁₋₃ aliphatic substituted by v³ instances of halogen. In some embodiments, each instance of R^{E1A} is independently C₁₋₃ aliphatic. In some embodiments, each instance of R^{E1A} is independently —CH₃, —CH₂F, —CHF₂—, or —CF₃. In some embodiments, R^{E1A} is —CH₃.

In some embodiments, R^{E1A} is selected from the groups depicted in the compounds in Table 1.

As defined generally above, each instance of R^{GA} is independently R^{F} or R^{H} substituted by v⁴ instances of R^{GC}. In some embodiments, each instance of R^{GA} is independently R^{F}. In some embodiments, each instance of R^{GA} is independently R^{H} substituted by v⁴ instances of R^{EC}.

In some embodiments, each instance of R^{GA} is independently C₁₋₆ aliphatic substituted by v⁴ instances of R^{GC}. In some embodiments, each instance of R^{GA} is independently C₁₋₃ aliphatic substituted by v⁴ instances of R^{GC}. In some embodiments, each instance of R^{G}A is independently C₁₋₃ aliphatic substituted by v⁴ instances of halogen. In some embodiments, each instance of R^{GA} is independently C₁₋₃ aliphatic. In some embodiments, each instance of R^{GA} is independently —CH₃, —CH₂F, —CHF₂—, or —CF₃. In some embodiments, R^{GA} is —CH₃.

In some embodiments, each instance of R^{GA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein said ring is substituted by v⁴ instances of R^{GC}.

In some embodiments, each instance of R^{GA} is independently a 5-6 membered monocyclic heteroaryl ring having 1-4 heteroatoms independently selected from nitrogen, oxygen, and sulfur; wherein said ring is substituted by v⁴ instances of R^{GC}. In some embodiments, each instance of R^{GA} is independently a 5-6 membered monocyclic heteroaryl ring having 1-3 nitrogen atoms; wherein said ring is substituted by v⁴ instances of R^{GC}. In some embodiments, each instance of R^{GA} is independently a 5-membered monocyclic heteroaryl ring having 1-3 nitrogen atoms.

In some embodiments, each instance of R^{GA} is independently a 3-7 membered saturated or partially unsaturated monocyclic heterocyclic ring having 1-2 heteroatoms independently selected from nitrogen, oxygen, and sulfur; wherein said ring is substituted by v⁴ instances of R^{GC}. In some embodiments, each instance of R^{GA} is independently a 4-6 membered saturated monocyclic heterocyclic ring having one nitrogen atom and optionally one additional heteroatom selected from nitrogen, oxygen, and sulfur; wherein said ring is substituted by v⁴ instances of R^{GC}. In some embodiments, each instance of R^{GA} is independently a 4-6 membered saturated monocyclic heterocyclic ring having one nitrogen atom and optionally one additional heteroatom selected from nitrogen, oxygen, and sulfur. In some embodiments, each instance of R^{GA} is independently pyrrolidin-1-yl, piperidin-1-yl, morpholin-4-yl, or piperazin-1-yl; each of which is substituted by v⁴ instances of R^{GC}. In some embodiments, each instance of R^{GA} is independently pyrrolidin-1-yl, piperidin-1-yl, morpholin-4-yl, or piperazin-1-yl.

In some embodiments, R^{GA} is selected from the groups depicted in the compounds in Table 1.

As defined generally above, R^{Q1A} is R^{F} or R^{H} substituted by v⁵ instances of R^{Q1C}. In some embodiments, R^{Q1A} is R^{F}. In some embodiments, R^{Q1A} is R^{F} substituted by v⁵ instances of R^{Q1C}.

In some embodiments, R^{Q1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{Q1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Q1A} is oxo. In some embodiments, R^{Q1A} is halogen. In some embodiments, R^{Q1A} is —CN. In some embodiments, R^{Q1A} is —NO₂. In some embodiments, R^{Q1A} is —OR. In some embodiments, R^{Q1A} is —SR. In some embodiments, R^{Q1A} is —NR₂. In some embodiments, R^{Q1A} is —S(O)₂R. In some embodiments, R^{Q1A} is —S(O)₂NR₂. In some embodiments, R^{Q1A} is —S(O)₂F. In some embodiments, R^{Q1A} is —S(O)R. In some embodiments, R^{Q1A} is —S(O)NR₂. In some embodiments, R^{Q1A} is —S(O)(NR)R. In some embodiments, R^{Q1A} is —C(O)R. In some embodiments, R^{Q1A} is —C(O)OR. In some embodiments, R^{Q1A} is —C(O)NR₂. In some embodiments, R^{Q1A} is —C(O)N(R)OR. In some embodiments, R^{Q1A} is —OC(O)R. In some embodiments, R^{Q1A} is —OC(O)NR₂. In some embodiments, R^{Q1A} is —N(R)C(O)OR. In some embodiments, R^{Q1A} is —N(R)C(O)R. In some embodiments, R^{Q1A} is —N(R)C(O)NR₂. In some embodiments, R^{Q1A} is —N(R)C(NR)NR₂. In some embodiments, R^{Q1A} is —N(R)S(O)₂NR₂. In some embodiments, R^{Q1A} is —N(R)S(O)₂R. In some embodiments, R^{Q1A} is —P(O)R₂. In some embodiments, R^{Q1A} is —P(O)(R)OR. In some embodiments, R^{Q1A} is —B(OR)₂. In some embodiments, R^{Q1A} is deuterium.

In some embodiments, R^{Q1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Q1A} is halogen, —CN, or —NO₂. In some embodiments, R^{Q1A} is —OR, —SR, or —NR₂. In some embodiments, R^{Q1A} is —S(O)₂R, —S(O)₂NR₂, —S(O)₂F, —S(O)R, —S(O)NR₂, or —S(O)(NR)R. In some embodiments, R^{Q1A} is —C(O)R, —C(O)OR, —C(O)NR₂, or —C(O)N(R)OR. In some embodiments, R^{Q1A} is —OC(O)R or —OC(O)NR₂. In some embodiments, R^{Q1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{Q1A} is —P(O)R₂ or —P(O)(R)OR.

In some embodiments, R^{Q1A} is —OR, —OC(O)R, or —OC(O)NR₂. In some embodiments, R^{Q1A} is —SR, —S(O)₂R, —S(O)₂NR₂, —S(O)₂F, —S(O)R, —S(O)NR₂, or —S(O)(NR)R. In some embodiments, R^{Q1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{Q1A} is —S(O)₂R, —S(O)₂NR₂, or —S(O)₂F. In some embodiments, R^{Q1A} is —S(O)R, —S(O)NR₂, or —S(O)(NR)R. In some embodiments, R^{Q1A} is —SR, —S(O)₂R, or —S(O)R. In some embodiments, R^{Q1A} is —S(O)₂NR₂, —S(O)NR₂, or —S(O)(NR)R. In some embodiments, R^{Q1A} is —S(O)₂NR₂ or —S(O)NR₂. In some embodiments, R^{Q1A} is —SR, —S(O)₂R, —S(O)₂NR₂, or —S(O)R.

In some embodiments, R^{Q1A} is —N(R)C(O)OR, —N(R)C(O)R, or —N(R)C(O)NR₂. In some embodiments, R^{Q1A} is —N(R)S(O)₂NR₂ or —N(R)S(O)₂R. In some embodiments, R^{QA} is —N(R)C(O)OR or —N(R)C(O)R. In some embodiments, R^{Q1A} is —N(R)C(O)NR₂ or —N(R)S(O)₂NR₂. In some embodiments, R^{Q1A} is —N(R)C(O)OR, —N(R)C(O)R, or —N(R)S(O)₂R.

In some embodiments, R^{Q1A} is —NR₂, —N(R)C(O)OR, —N(R)C(O)R, or —N(R)C(O)NR₂. In some embodiments, R^{Q1A} is —NR₂, —N(R)C(O)OR, or —N(R)C(O)R. In some embodiments, R^{Q1A} is —NR₂, —N(R)C(O)OR, —N(R)C(O)R, or —N(R)S(O)₂R.

In some embodiments, R^{Q1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is a C₁₋₆ aliphatic chain substituted by v⁵ instances of R^{Q1C}. In some embodiments, R^{Q1A} is phenyl substituted by v⁵ instances of R^{Q1C}. In some embodiments, R^{Q1A} is naphthyl substituted by v⁵ instances of R^{Q1C}. In some embodiments, R^{Q1A} is a 5-6 membered monocyclic heteroaryl ring having 1-4 heteroatoms independently selected from nitrogen, oxygen, and sulfur; wherein said ring is substituted by v⁵ instances of R^{Q1C}. In some embodiments, R^{Q1A} is an 8-10 membered bicyclic heteroaryl ring having 1-4 heteroatoms independently selected from nitrogen, oxygen, and sulfur; wherein said ring is substituted by v⁵ instances of R^{Q1C}. In some embodiments, R^{Q1A} is a 3-7 membered saturated or partially unsaturated monocyclic carbocyclic ring substituted by v⁵ instances of R^{Q1C}. In some embodiments, R^{Q1A} is a 5-12 membered saturated or partially unsaturated bicyclic carbocyclic ring substituted by v⁵ instances of R^{Q1C}. In some embodiments, R^{Q1A} is a 3-7 membered saturated or partially unsaturated monocyclic heterocyclic ring having 1-2 heteroatoms independently selected from nitrogen, oxygen, and sulfur; wherein said ring is substituted by v⁵ instances of R^{Q1C}. In some embodiments, R^{Q1A} is a 7-12 membered saturated or partially unsaturated bicyclic heterocyclic ring having 1-4 heteroatoms independently selected from nitrogen, oxygen, and sulfur; wherein said ring is substituted by v⁵ instances of R^{Q1C}.

In some embodiments, R^{Q1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁵ instances of R^{Q1C}. In some embodiments, R^{Q1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by v⁵ instances of R^{Q1C}. In some embodiments, R^{Q1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁵ instances of R^{Q1C}. In some embodiments, R^{Q1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl or naphthyl; each of which is substituted by v⁵ instances of R^{Q1C}. In some embodiments, R^{Q1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁵ instances of R^{Q1C}. In some embodiments, R^{Q1A} is a 3-7 membered saturated or partially unsaturated monocyclic carbocyclic ring or a 5-12 membered saturated or partially unsaturated bicyclic carbocyclic ring; each of which is substituted by v⁵ instances of R^{Q1C}. In some embodiments, R^{Q1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl or a 5-6 membered monocyclic heteroaryl ring having 1-4 heteroatoms independently selected from nitrogen, oxygen, and sulfur; each of which is substituted by v⁵ instances of R^{Q1C}. In some embodiments, R^{Q1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by v⁵ instances of R^{Q1C}. In some embodiments, R^{Q1A} is naphthyl or an 8-10 membered bicyclic heteroaryl ring having 1-4 heteroatoms independently selected from nitrogen, oxygen, and sulfur; each of which is substituted by v⁵ instances of R^{Q1C}. In some embodiments, R^{Q1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}.

In some embodiments, R^{Q1A} is phenyl or a 3-7 membered saturated or partially unsaturated monocyclic carbocyclic ring; each of which is substituted by v⁵ instances of R^{Q1C}. In some embodiments, R^{Q1A} is naphthyl or a 5-12 membered saturated or partially unsaturated bicyclic carbocyclic ring; each of which is substituted by v⁵ instances of R^{Q1C}. In some embodiments, R^{Q1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v⁵ instances of R^{Q1C}. In some embodiments, R^{Q1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by vs instances of R^{Q1C}.

In some embodiments, R^{Q1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁵ instances of R^{Q1C}. In some embodiments, R^{Q1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by v⁵ instances of R^{Q1C}. In some embodiments, R^{Q1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁵ instances of R^{Q1C}.

In some embodiments, R^{Q1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by v⁵ instances of R^{Q1C}. In some embodiments, R^{Q1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by v⁵ instances of R^{Q1C}. In some embodiments, R^{Q1A} is a C₁₋₆ aliphatic chain, phenyl, or a 3-7 membered saturated or partially unsaturated monocyclic carbocyclic ring; each of which is substituted by v⁵ instances of R^{Q1C}.

In some embodiments, R^{Q1A} is selected from the groups depicted in the compounds in Table 1.

As defined generally above, R^{Y1A} is R^{F} or R^{H} substituted by v⁶ instances of R^{Y1C}. In some embodiments, R^{Y1A} is R^{F}. In some embodiments, R^{Y1A} is R^{H} substituted by v⁶ instances of R^{Y1C}.

In some embodiments, R^{Y1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{Y1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Y1A} is oxo. In some embodiments, R^{Y1A} is halogen. In some embodiments, R^{Y1A} is —CN. In some embodiments, R^{Y1A} is —NO₂. In some embodiments, R^{Y1A} is —OR. In some embodiments, R^{Y1A} is —SR. In some embodiments, R^{Y1A} is —NR₂. In some embodiments, R^{Y1A} is —S(O)₂R. In some embodiments, R^{Y1A} is —S(O)₂NR₂. In some embodiments, R^{Y1A} is —S(O)₂F. In some embodiments, R^{Y1A} is —S(O)R. In some embodiments, R^{Y1A} is —S(O)NR₂. In some embodiments, R^{Y1A} is —S(O)(NR)R. In some embodiments, R^{Y1A} is —C(O)R. In some embodiments, R^{Y1A} is —C(O)OR. In some embodiments, R^{Y1A} is —C(O)NR₂. In some embodiments, R^{Y1A} is —C(O)N(R)OR. In some embodiments, R^{Y1A} is —OC(O)R. In some embodiments, R^{Y1A} is —OC(O)NR₂. In some embodiments, R^{Y1A} is —N(R)C(O)OR. In some embodiments, R^{Y1A} is —N(R)C(O)R. In some embodiments, R^{Y1A} is —N(R)C(O)NR₂. In some embodiments, R^{Y1A} is —N(R)C(NR)NR₂. In some embodiments, R^{Y1A} is —N(R)S(O)₂NR₂. In some embodiments, R^{Y1A} is —N(R)S(O)₂R. In some embodiments, R^{Y1A} is —P(O)R₂. In some embodiments, R^{Y1A} is —P(O)(R)OR. In some embodiments, R^{Y1A} is —B(OR)₂. In some embodiments, R^{Y1A} is deuterium.

In some embodiments, R^{Y1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Y1A} is halogen, —CN, or —NO₂. In some embodiments, R^{Y1A} is —OR, —SR, or —NR₂. In some embodiments, R^{Y1A} is —S(O)₂R, —S(O)₂NR₂, —S(O)₂F, —S(O)R, —S(O)NR₂, or —S(O)(NR)R. In some embodiments, R^{Y1A} is —C(O)R, —C(O)OR, —C(O)NR₂, or —C(O)N(R)OR. In some embodiments, R^{Y1A} is —OC(O)R or —OC(O)NR₂. In some embodiments, R^{Y1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{Y1A} is —P(O)R₂ or —P(O)(R)OR.

In some embodiments, R^{Y1A} is —OR, —OC(O)R, or —OC(O)NR₂. In some embodiments, R^{Y1A} is —SR, —S(O)₂R, —S(O)₂NR₂, —S(O)₂F, —S(O)R, —S(O)NR₂, or —S(O)(NR)R. In some embodiments, R^{Y1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{Y1A} is —S(O)₂R, —S(O)₂NR₂, or —S(O)₂F. In some embodiments, R^{Y1A} is —S(O)R, —S(O)NR₂, or —S(O)(NR)R. In some embodiments, R^{Y1A} is —SR, —S(O)₂R, or —S(O)R. In some embodiments, R^{Y1A} is —S(O)₂NR₂, —S(O)NR₂, or —S(O)(NR)R. In some embodiments, R^{Y1A} is —S(O)₂NR₂ or —S(O)NR₂. In some embodiments, R^{Y1A} is —SR, —S(O)₂R, —S(O)₂NR₂, or —S(O)R.

In some embodiments, R^{Y1A} is —N(R)C(O)OR, —N(R)C(O)R, or —N(R)C(O)NR₂. In some embodiments, R^{Y1A} is —N(R)S(O)₂NR₂ or —N(R)S(O)₂R. In some embodiments, R^{YA} is —N(R)C(O)OR or —N(R)C(O)R. In some embodiments, R^{Y1A} is —N(R)C(O)NR₂ or —N(R)S(O)₂NR₂. In some embodiments, R^{Y1A} is —N(R)C(O)OR, —N(R)C(O)R, or —N(R)S(O)₂R.

In some embodiments, R^{Y1A} is —NR₂, —N(R)C(O)OR, —N(R)C(O)R, or —N(R)C(O)NR₂. In some embodiments, R^{Y1A} is —NR₂, —N(R)C(O)OR, or —N(R)C(O)R. In some embodiments, R^{Y1A} is —NR₂, —N(R)C(O)OR, —N(R)C(O)R, or —N(R)S(O)₂R.

In some embodiments, R^{Y1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is a C₁₋₆ aliphatic chain substituted by v⁶ instances of R^{Y1C}. In some embodiments, R^{Y1A} is phenyl substituted by v⁶ instances of R^{Y1C}. In some embodiments, R^{Y1A} is naphthyl substituted by v⁶ instances of R^{Y1C}. In some embodiments, R^{Y1A} is a 5-6 membered monocyclic heteroaryl ring having 1-4 heteroatoms independently selected from nitrogen, oxygen, and sulfur; wherein said ring is substituted by v⁶ instances of R^{Y1C}. In some embodiments, R^{Y1A} is an 8-10 membered bicyclic heteroaryl ring having 1-4 heteroatoms independently selected from nitrogen, oxygen, and sulfur; wherein said ring is substituted by v⁶ instances of R^{Y1C}. In some embodiments, R^{Y1A} is a 3-7 membered saturated or partially unsaturated monocyclic carbocyclic ring substituted by v⁶ instances of R^{Y1C}. In some embodiments, R^{Y1A} is a 5-12 membered saturated or partially unsaturated bicyclic carbocyclic ring substituted by v⁶ instances of R^{Y1C}. In some embodiments, R^{Y1A} is a 3-7 membered saturated or partially unsaturated monocyclic heterocyclic ring having 1-2 heteroatoms independently selected from nitrogen, oxygen, and sulfur; wherein said ring is substituted by v⁶ instances of R^{Y1C}. In some embodiments, R^{Y1A} is a 7-12 membered saturated or partially unsaturated bicyclic heterocyclic ring having 1-4 heteroatoms independently selected from nitrogen, oxygen, and sulfur; wherein said ring is substituted by v⁶ instances of R^{Y1C}.

In some embodiments, R^{Y1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁶ instances of R^{Y1C}. In some embodiments, R^{Y1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by v⁶ instances of R^{Y1C}. In some embodiments, R^{Y1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁶ instances of R^{Y1C}. In some embodiments, R^{Y1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl or naphthyl; each of which is substituted by v⁶ instances of R^{Y1C}. In some embodiments, R^{Y1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁶ instances of R^{Y1C}. In some embodiments, R^{Y1A} is a 3-7 membered saturated or partially unsaturated monocyclic carbocyclic ring or a 5-12 membered saturated or partially unsaturated bicyclic carbocyclic ring; each of which is substituted by v⁶ instances of R^{Y1C}. In some embodiments, R^{Y1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl or a 5-6 membered monocyclic heteroaryl ring having 1-4 heteroatoms independently selected from nitrogen, oxygen, and sulfur; each of which is substituted by v⁶ instances of R^{Y1C}. In some embodiments, R^{Y1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by v⁶ instances of R^{Y1C}. In some embodiments, R^{Y1A} is naphthyl or an 8-10 membered bicyclic heteroaryl ring having 1-4 heteroatoms independently selected from nitrogen, oxygen, and sulfur; each of which is substituted by v⁶ instances of R^{Y1C}. In some embodiments, R^{Y1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is phenyl or a 3-7 membered saturated or partially unsaturated monocyclic carbocyclic ring; each of which is substituted by v⁶ instances of R^{Y1C}. In some embodiments, R^{Y1A} is naphthyl or a 5-12 membered saturated or partially unsaturated bicyclic carbocyclic ring; each of which is substituted by v⁶ instances of R^{Y1C}. In some embodiments, R^{Y1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v⁶ instances of R^{Y1C}. In some embodiments, R^{Y1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}.

In some embodiments, R^{Y1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁶ instances of R^{Y1C}. In some embodiments, R^{Y1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by v⁶ instances of R^{Y1C}. In some embodiments, R^{Y1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁶ instances of R^{Y1C}.

In some embodiments, R^{Y1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by v⁶ instances of R^{Y1C}. In some embodiments, R^{Y1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by v⁶ instances of R^{Y1C}. In some embodiments, R^{Y1A} is a C₁₋₆ aliphatic chain, phenyl, or a 3-7 membered saturated or partially unsaturated monocyclic carbocyclic ring; each of which is substituted by v⁶ instances of R^{Y1C}.

In some embodiments, R^{Y1A} is selected from the groups depicted in the compounds in Table 1.

As defined generally above, R^{Y2A} is R^{F} or R^{H} substituted by v instances of R^{Y2C}. In some embodiments, R^{Y2A} is R^{F}. In some embodiments, R^{Y2A} is R^{H} substituted by v⁷ instances of R^{Y2C}.

In some embodiments, R^{Y2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁷ instances of R^{Y2C}.

In some embodiments, R^{Y2A} is a C₁₋₆ aliphatic chain substituted by v⁷ instances of R^{Y2C}. In some embodiments, R^{Y2A} is phenyl substituted by v⁷ instances of R^{Y2C}. In some embodiments, R^{Y2A} is naphthyl substituted by v⁷ instances of R^{Y2C}. In some embodiments, R^{Y2A} is cubanyl substituted by v⁷ instances of R^{Y2C}. In some embodiments, R^{Y2A} is adamantyl substituted by v⁷ instances of R^{Y2C}. In some embodiments, R^{Y2A} is a 5-6 membered monocyclic heteroaryl ring having 1-4 heteroatoms independently selected from nitrogen, oxygen, and sulfur; wherein said ring is substituted by v⁷ instances of R^{Y2C}. In some embodiments, R^{Y2A} is an 8-10 membered bicyclic heteroaryl ring having 1-4 heteroatoms independently selected from nitrogen, oxygen, and sulfur; wherein said ring is substituted by v⁷ instances of R^{Y2C}. In some embodiments, R^{Y2A} is a 3-7 membered saturated or partially unsaturated monocyclic carbocyclic ring; wherein said ring is substituted by v⁷ instances of R^{Y2C}. In some embodiments, R^{Y2A} is a 5-12 membered saturated or partially unsaturated bicyclic carbocyclic ring; wherein said ring is substituted by v⁷ instances of R^{Y2C}. In some embodiments, R^{Y2A} is a 3-7 membered saturated or partially unsaturated monocyclic heterocyclic ring having 1-2 heteroatoms independently selected from nitrogen, oxygen, and sulfur; wherein said ring is substituted by v⁷ instances of R^{Y2C}. In some embodiments, R^{Y2A} is a 7-12 membered saturated or partially unsaturated bicyclic heterocyclic ring having 1-4 heteroatoms independently selected from nitrogen, oxygen, and sulfur; wherein said ring is substituted by v⁷ instances of R^{Y2C}.

In some embodiments, R^{Y2A} is a C₁₋₆ aliphatic chain; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁷ instances of R^{Y2C}. In some embodiments, R^{Y2A} is a C₁₋₆ aliphatic chain or a 3-7 membered saturated or partially unsaturated monocyclic carbocyclic ring; each of which is substituted by v⁷ instances of R^{Y2C}. In some embodiments, R^{Y2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v⁷ instances of R^{Y2C}.

In some embodiments, R^{Y2A} is a 5-6 membered monocyclic heteroaryl ring having 1-4 heteroatoms independently selected from nitrogen, oxygen, and sulfur; wherein said ring is substituted by v⁷ instances of R^{Y2C}.

In some embodiments, R^{Y2A} is a 6-membered monocyclic heteroaryl ring having 1-4 heteroatoms independently selected from nitrogen, oxygen, and sulfur; wherein said ring is substituted by v⁷ instances of R^{Y2C}. In some embodiments, R^{Y2A} is a 6-membered monocyclic heteroaryl ring having 1 or 2 nitrogen atoms; wherein said ring is substituted by v⁷ instances of R^{Y2C}. In some embodiments, R^{Y2A} is pyridinyl substituted by v instances of R^{Y2C}. In some embodiments, R^{Y2A} is pyridin-3-yl substituted by v instances of R^{Y2C}. In some embodiments, R^{Y2A} is pyridin-3-yl substituted by one R^{Y2C}. In some embodiments, R^{Y2A} is pyridin-2-yl substituted by v instances of R^{Y2C}. In some embodiments, R^{Y2A} is pyridin-2-yl substituted by one R^{Y2C}. In some embodiments, R^{Y2A} is pyrimidinyl or pyridazinyl substituted by v⁷ instances of R^{Y2C}. In some embodiments, R^{Y2A} is pyrimidin-2-yl. In some embodiments, R^{Y2A} is pyridazin-3-yl.

In some embodiments, R^{Y2A} is a 5-membered monocyclic heteroaryl ring having 1-4 heteroatoms independently selected from nitrogen, oxygen, and sulfur; wherein said ring is substituted by v⁷ instances of R^{Y2C}. In some embodiments, R^{Y2A} is a 5-membered monocyclic heteroaryl ring having 1-2 heteroatoms independently selected from nitrogen and oxygen; wherein said ring is substituted by v⁷ instances of R^{Y2C}. In some embodiments, R^{Y2A} is isoxazolyl, oxazolyl, pyrazolyl, imidazolyl, or triazolyl; each of which is substituted by v⁷ instances of R^{Y2C}. In some embodiments, R^{Y2A} is isoxazolyl, oxazolyl, pyrazolyl, imidazolyl, or triazolyl; each of which is substituted by one R^{Y2C}. In some embodiments, R^{Y2A} is isoxazolyl, oxazolyl, pyrazolyl, imidazolyl, or triazolyl. In some embodiments, R^{Y2A} is isoxazolyl, oxazolyl, pyrazolyl, or imidazolyl; each of which is substituted by v⁷ instances of R^{Y2C}.

In some embodiments, R^{Y2A} is isoxazolyl or oxazolyl; each of which is substituted by v⁷ instances of R^{Y2C}. In some embodiments, R^{Y2A} is isoxazolyl substituted by v instances of R^{Y2C}. In some embodiments, R^{Y2A} is isoxazolyl. In some embodiments, R^{Y2A} is oxazolyl substituted by v⁷ instances of R^{Y2C}. In some embodiments, R^{Y2A} is oxazolyl. In some embodiments, R^{Y2A} is pyrazolyl or imidazolyl; each of which is substituted by v⁷ instances of R^{Y2C}. In some embodiments, R^{Y2A} is pyrazolyl substituted by v⁷ instances of R^{Y2C}. In some embodiments, R^{Y2A} is pyrazolyl. In some embodiments, R^{Y2A} is imidazolyl substituted by v⁷ instances of R^{Y2C}. In some embodiments, R^{Y2A} is imidazolyl. In some embodiments, R^{Y2A} is triazolyl substituted by v⁷ instances of R^{Y2C}. In some embodiments, R^{Y2A} is triazolyl.

In some embodiments, R^{Y2A} is

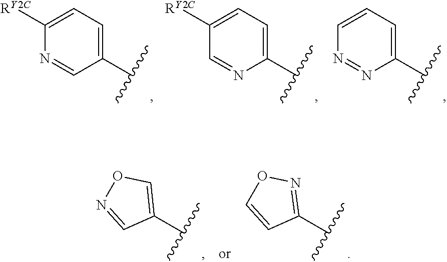

In some embodiments, R^{Y2A} is

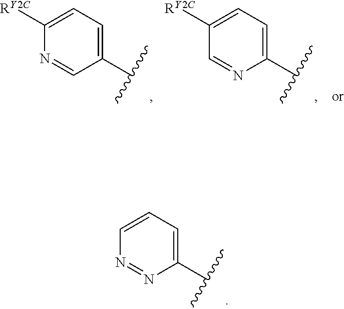

In some embodiments, R^{Y2A} is

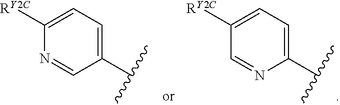

In some embodiments, R^{Y2A} is

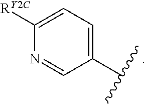

In some embodiments, R^{Y2A} is

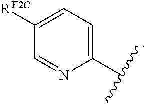

In some embodiments, R^{Y2A} is

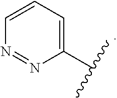 [SMILES: *c1cccnn1 ; "."]

In some embodiments, R^{Y2A} is

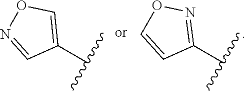 [SMILES: *c1cnoc1 ; "or" ; *c1ccon1 ; "."]

In some embodiments, R^{Y2A} is

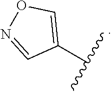 [SMILES: *c1cnoc1 ; "."]

In some embodiments, R^{Y2A} is

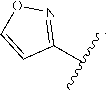 [SMILES: *c1ccon1 ; "."]

In some embodiments, R^{Y2A} is a 3-7 membered saturated or partially unsaturated monocyclic heterocyclic ring having 1-2 heteroatoms independently selected from nitrogen, oxygen, and sulfur; wherein said ring is substituted by v⁷ instances of R^{Y2C}. In some embodiments, R^{Y2A} is a 4-6 membered saturated or partially unsaturated monocyclic heterocyclic ring having 1-2 heteroatoms independently selected from nitrogen, oxygen, and sulfur; wherein said ring is substituted by v⁷ instances of R^{Y2C}. In some embodiments, R^{Y2A} is a 4-6 membered saturated or partially unsaturated monocyclic heterocyclic ring having 1-2 heteroatoms independently selected from nitrogen, oxygen, and sulfur.

In some embodiments, R^{Y2A} is a 4-6 membered saturated monocyclic heterocyclic ring having 1-2 oxygen atoms; wherein said ring is substituted by v⁷ instances of R^{Y2C}. In some embodiments, R^{Y2A} is a 4-6 membered saturated monocyclic heterocyclic ring having 1-2 oxygen atoms. In some embodiments, R^{Y2A} is oxetanyl, tetrahydrofuranyl, tetrahydropyranyl, or 1,4-dioxan-2-yl, each of which is substituted by v⁷ instances of R^{Y2C}In some embodiments, R^{Y2A} is oxetanyl, tetrahydrofuranyl, tetrahydropyranyl, or 1,4-dioxan-2-yl. In some embodiments, R^{Y2A} is oxetanyl, tetrahydrofuranyl, or tetrahydropyranyl, each of which is substituted by v⁷ instances of R^{Y2C}. In some embodiments, R^{Y2A} is oxetanyl, tetrahydrofuranyl, or tetrahydropyranyl. In some embodiments, R^{Y2A} is oxetanyl. In some embodiments, R^{Y2A} is tetrahydrofuranyl. In some embodiments, R^{Y2A} is tetrahydropyranyl. In some embodiments, R^{Y2A} is 1,4-dioxan-2-yl.

In some embodiments, R^{Y2A} is

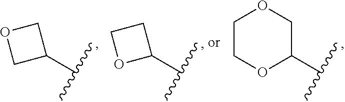

In some embodiments, R^{Y2A} is

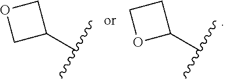 [SMILES: *C1COC1 ; "or" ; *C1CCO1 ; "."]

In some embodiments, R^{Y2A} is

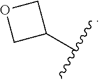 [SMILES: *C1COC1 ; "."]

In some embodiments, R^{Y2A} is

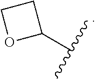 [SMILES: *C1CCO1 ; "."]

In some embodiments, R^{Y2A} is

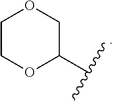 [SMILES: *C1COCCO1 ; "."]

In some embodiments, R^{Y2A} is a C₁₋₆ aliphatic substituted by v⁷ instances of R^{Y2C}. In some embodiments, R^{Y2A} is a C₁₋₆ aliphatic chain substituted by v⁷ instances of R^{Y2C}. In some embodiments, R^{Y2A} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, R^{Y2A} is a C₁₋₆ aliphatic that is (i) substituted with 1 or 2 groups independently selected from —O—(C₁₋₆ aliphatic), —OH, —N(C₁₋₆ aliphatic)₂, and —CN; and (ii) optionally substituted with 1, 2, or 3 halogen atoms. In some embodiments, R^{Y2A} is a C₁₋₆ aliphatic substituted with one —OH and 1, 2, or 3 halogen atoms. In some embodiments, R^{Y2A} is

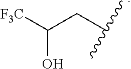 [SMILES: "." ; *CC(O)C(F)(F)F]

In some embodiments, R^{Y2A} is a C₁₋₆ aliphatic substituted with one —CN. In some embodiments, R^{Y2A} is —(CH₂)₂CN. In some embodiments, R^{Y2A} is a C₁₋₆ aliphatic substituted with 1, 2, or 3 halogen atoms. In some embodiments, R^{Y2A} is —CH₂CHF₂. In some embodiments, R^{Y2A} is a C₁₋₆ aliphatic substituted with 1, 2, or 3 deuterium atoms. In some embodiments, R^{Y2A} is a C₁₋₆ aliphatic. In some embodiments, R^{Y2A} is a C₁₋₄ alkyl. In some embodiments, R^{Y2A} is —CH₃. In some embodiments, R^{Y2A} is —CD₃.

In some embodiments, R^{Y2A} is

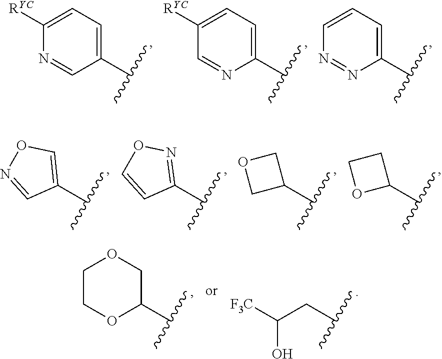

In some embodiments, R^{Y2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R^{Y2A} is halogen, —CN, —S(O)₂R, —S(O)₂NR₂, —S(O)₂F, —S(O)R, —S(O)NR₂, —S(O)(NR)R, —C(O)R, —C(O)OR, —C(O)NR₂, or —C(O)N(R)OR. In some embodiments, R^{Y2A} is halogen or —CN.

In some embodiments, R^{Y2A} is selected from the groups depicted in the compounds in Table 1.

As defined generally above, R^{L2} is R^{F} or R^{H} substituted by v⁸ instances of R^{L2C}. In some embodiments, R^{L2} is R^{F}. In some embodiments, R^{L2} is R^{H} substituted by v⁸ instances of R^{L2C}.

In some embodiments, R^{L2} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L2} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L2} is oxo. In some embodiments, R^{L2} is halogen. In some embodiments, R^{L2} is —CN. In some embodiments, R^{L2} is —NO₂. In some embodiments, R^{L2} is —OR. In some embodiments, R^{L2} is —SR. In some embodiments, R^{L2} is —NR₂. In some embodiments, R^{L2} is —S(O)₂R. In some embodiments, R^{L2} is —S(O)₂NR₂. In some embodiments, R^{L2} is —S(O)₂F. In some embodiments, R^{L2} is —S(O)R. In some embodiments, R^{L2} is —S(O)NR₂. In some embodiments, R^{L2} is —S(O)(NR)R. In some embodiments, R^{L2} is —C(O)R. In some embodiments, R^{L2} is —C(O)OR. In some embodiments, R^{L2} is —C(O)NR₂. In some embodiments, R^{L2} is —C(O)N(R)OR. In some embodiments, R^{L2} is —OC(O)R. In some embodiments, R^{L2} is —OC(O)NR₂. In some embodiments, R^{L2} is —N(R)C(O)OR. In some embodiments, R^{L2} is —N(R)C(O)R. In some embodiments, R^{L2} is —N(R)C(O)NR₂. In some embodiments, R^{L2} is —N(R)C(NR)NR₂. In some embodiments, R^{L2} is —N(R)S(O)₂NR₂. In some embodiments, R^{L2} is —N(R)S(O)₂R. In some embodiments, R^{L2} is —P(O)R₂. In some embodiments, R^{L2} is —P(O)(R)OR. In some embodiments, R^{L2} is —B(OR)₂. In some embodiments, R^{L2} is deuterium.

In some embodiments, R^{L2} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L2} is halogen, —CN, or —NO₂. In some embodiments, R^{L2} is —OR, —SR, or —NR₂. In some embodiments, R^{L2} is —S(O)₂R, —S(O)₂NR₂, —S(O)₂F, —S(O)R, —S(O)NR₂, or —S(O)(NR)R. In some embodiments, R^{L2} is —C(O)R, —C(O)OR, —C(O)NR₂, or —C(O)N(R)OR. In some embodiments, R^{L2} is —OC(O)R or —OC(O)NR₂. In some embodiments, R^{L2} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L2} is —P(O)R₂ or —P(O)(R)OR.

In some embodiments, R^{L2} is —OR, —OC(O)R, or —OC(O)NR₂. In some embodiments, R^{L2} is —SR, —S(O)₂R, —S(O)₂NR₂, —S(O)₂F, —S(O)R, —S(O)NR₂, or —S(O)(NR)R. In some embodiments, R^{L2} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L2} is —S(O)₂R, —S(O)₂NR₂, or —S(O)₂F. In some embodiments, R^{L2} is —S(O)R, —S(O)NR₂, or —S(O)(NR)R. In some embodiments, R^{L2} is —SR, —S(O)₂R, or —S(O)R. In some embodiments, R^{L2} is —S(O)₂NR₂, —S(O)NR₂, or —S(O)(NR)R. In some embodiments, R^{L2} is —S(O)₂NR₂ or —S(O)NR₂. In some embodiments, R^{L2} is —SR, —S(O)₂R, —S(O)₂NR₂, or —S(O)R.

In some embodiments, R^{L2} is —N(R)C(O)OR, —N(R)C(O)R, or —N(R)C(O)NR₂. In some embodiments, R^{L2} is —N(R)S(O)₂NR₂ or —N(R)S(O)₂R. In some embodiments, R^{L2} is —N(R)C(O)OR or —N(R)C(O)R. In some embodiments, R^{L2} is —N(R)C(O)NR₂ or —N(R)S(O)₂NR₂. In some embodiments, R^{L2} is —N(R)C(O)OR, —N(R)C(O)R, or —N(R)S(O)₂R.

In some embodiments, R^{L2} is —NR₂, —N(R)C(O)OR, —N(R)C(O)R, or —N(R)C(O)NR₂. In some embodiments, R^{L2} is —NR₂, —N(R)C(O)OR, or —N(R)C(O)R. In some embodiments, R^{L2} is —NR₂, —N(R)C(O)OR, —N(R)C(O)R, or —N(R)S(O)₂R.

In some embodiments, R^{L2} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is a C₁₋₆ aliphatic chain substituted by v⁸ instances of R^{L2C}In some embodiments, R^{L2} is phenyl substituted by v⁸ instances of R^{L2C}. In some embodiments, R^{L2} is naphthyl substituted by v⁸ instances of R^{L2C}. In some embodiments, R^{L2} is a 5-6 membered monocyclic heteroaryl ring having 1-4 heteroatoms independently selected from nitrogen, oxygen, and sulfur; wherein said ring is substituted by v⁸ instances of R^{L2C}. In some embodiments, R^{L2} is an 8-10 membered bicyclic heteroaryl ring having 1-4 heteroatoms independently selected from nitrogen, oxygen, and sulfur; wherein said ring is substituted by v⁸ instances of R^{L2C}. In some embodiments, R^{L2} is a 3-7 membered saturated or partially unsaturated monocyclic carbocyclic ring substituted by v⁸ instances of R^{L2C}. In some embodiments, R^{L2} is a 5-12 membered saturated or partially unsaturated bicyclic carbocyclic ring substituted by v⁸ instances of R^{L2C}. In some embodiments, R^{L2} is a 3-7 membered saturated or partially unsaturated monocyclic heterocyclic ring having 1-2 heteroatoms independently selected from nitrogen, oxygen, and sulfur; wherein said ring is substituted by v⁸ instances of R^{L2C}. In some embodiments, R^{L2} is a 7-12 membered saturated or partially unsaturated bicyclic heterocyclic ring having 1-4 heteroatoms independently selected from nitrogen, oxygen, and sulfur; wherein said ring is substituted by v⁸ instances of R^{L2C}.

In some embodiments, R^{L2} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁸ instances of R^{L2C}. In some embodiments, R^{L2} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl; naphthyl; a 3-7 membered saturated or partially unsaturated monocyclic carbocyclic ring; or a 5-12 membered saturated or partially unsaturated bicyclic carbocyclic ring; each of which is substituted by v⁸ instances of R^{L2C}. In some embodiments, R^{L2} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁸ instances of R^{L2C}. In some embodiments, R^{L2} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl or naphthyl; each of which is substituted by v⁸ instances of R^{L2C}. In some embodiments, R^{L2} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by v⁸ instances of R^{L2C}. In some embodiments, R^{L2} is a 3-7 membered saturated or partially unsaturated monocyclic carbocyclic ring or a 5-12 membered saturated or partially unsaturated bicyclic carbocyclic ring; each of which is substituted by v⁸ instances of R^{L2C}. In some embodiments, R^{L2} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl or a 5-6 membered monocyclic heteroaryl ring having 1-4 heteroatoms independently selected from nitrogen, oxygen, and sulfur; each of which is substituted by v⁸ instances of R^{L2C}. In some embodiments, R^{L2} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by v⁸ instances of R^{L2C}. In some embodiments, R^{L2} is naphthyl or an 8-10 membered bicyclic heteroaryl ring having 1-4 heteroatoms independently selected from nitrogen, oxygen, and sulfur; each of which is substituted by v⁸ instances of R^{L2C}. In some embodiments, R^{L2} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is phenyl or a 3-7 membered saturated or partially unsaturated monocyclic carbocyclic ring; each of which is substituted by v⁸ instances of R^{L2C}In some embodiments, R^{L2} is naphthyl or a 5-12 membered saturated or partially unsaturated bicyclic carbocyclic ring; each of which is substituted by v⁸ instances of R^{L2C}. In some embodiments, R^{L2} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by v⁸ instances of R^{L2C}. In some embodiments, R^{L2} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}.

In some embodiments, R^{L2} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by v⁸ instances of R^{L2C}. In some embodiments, R^{L2} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by v⁸ instances of R^{L2C}. In some embodiments, R^{L2} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by v⁸ instances of R^{L2C}.

In some embodiments, R^{L2} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by v⁸ instances of R^{L2C}. In some embodiments, R^{L2} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by v⁸ instances of R^{L2C}. In some embodiments, R^{L2} is a C₁₋₆ aliphatic chain, phenyl, or a 3-7 membered saturated or partially unsaturated monocyclic carbocyclic ring; each of which is substituted by v⁸ instances of R^{L2C}.

In some embodiments, R^{L2} is selected from the groups depicted in the compounds in Table 1.

As defined generally above, each instance of R^{F} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{F} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{F} is oxo. In some embodiments, R^{F} is halogen. In some embodiments, R^{F} is —CN. In some embodiments, R^{F} is —NO₂. In some embodiments, R^{F} is —OR. In some embodiments, R^{F} is —SF₅. In some embodiments, R^{F} is —SR. In some embodiments, R^{F} is —NR₂. In some embodiments, R^{F} is —S(O)₂R. In some embodiments, R^{F} is —S(O)₂NR₂. In some embodiments, R^{F} is —S(O)₂F. In some embodiments, R^{F} is —S(O)R. In some embodiments, R^{F} is —S(O)NR₂. In some embodiments, R^{F} is —S(O)(NR)R. In some embodiments, R^{F} is —C(O)R. In some embodiments, R^{F} is —C(O)OR. In some embodiments, R^{F} is —C(O)NR₂. In some embodiments, R^{F} is —C(O)N(R)OR. In some embodiments, R^{F} is —OC(O)R. In some embodiments, R^{F} is —OC(O)NR₂. In some embodiments, R^{F} is —N(R)C(O)OR. In some embodiments, R^{F} is —N(R)C(O)R. In some embodiments, R^{F} is —N(R)C(O)NR₂. In some embodiments, R^{F} is —N(R)C(NR)NR₂. In some embodiments, R^{F} is —N(R)S(O)₂NR₂. In some embodiments, R^{F} is —N(R)S(O)₂R. In some embodiments, R^{F} is —P(O)R₂. In some embodiments, R^{F} is —P(O)(R)OR. In some embodiments, R^{F} is —B(OR)₂. In some embodiments, R^{F} is deuterium.

In some embodiments, R^{F} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{F} is halogen, —CN, or —NO₂. In some embodiments, R^{F} is —OR, —SR, or —NR₂. In some embodiments, R^{F} is —S(O)₂R, —S(O)₂NR₂, —S(O)₂F, —S(O)R, —S(O)NR₂, or —S(O)(NR)R. In some embodiments, R^{F} is —C(O)R, —C(O)OR, —C(O)NR₂, or —C(O)N(R)OR. In some embodiments, R^{F} is —OC(O)R or —OC(O)NR₂. In some embodiments, R^{F} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{F} is —P(O)R₂ or —P(O)(R)OR.

In some embodiments, R^{F} is —OR, —OC(O)R, or —OC(O)NR₂. In some embodiments, R^{F} is —SR, —S(O)₂R, —S(O)₂NR₂, —S(O)₂F, —S(O)R, —S(O)NR₂, or —S(O)(NR)R. In some embodiments, R^{F} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{F} is —S(O)₂R, —S(O)₂NR₂, or —S(O)₂F. In some embodiments, R^{F} is —S(O)R, —S(O)NR₂, or —S(O)(NR)R. In some embodiments, R^{F} is —SR, —S(O)₂R, or —S(O)R. In some embodiments, R^{F} is —S(O)₂NR₂, —S(O)NR₂, or —S(O)(NR)R. In some embodiments, R^{F} is —S(O)₂NR₂ or —S(O)NR₂. In some embodiments, R^{F} is —SR, —S(O)₂R, —S(O)₂NR₂, or —S(O)R.

In some embodiments, R^{F} is —N(R)C(O)OR, —N(R)C(O)R, or —N(R)C(O)NR₂. In some embodiments, R^{F} is —N(R)S(O)₂NR₂ or —N(R)S(O)₂R. In some embodiments, R^{F} is —N(R)C(O)OR or —N(R)C(O)R. In some embodiments, R^{F} is —N(R)C(O)NR₂ or —N(R)S(O)₂NR₂. In some embodiments, R^{F} is —N(R)C(O)OR, —N(R)C(O)R, or —N(R)S(O)₂R.

In some embodiments, R^{F} is —NR₂, —N(R)C(O)OR, —N(R)C(O)R, or —N(R)C(O)NR₂. In some embodiments, R^{F} is —NR₂, —N(R)C(O)OR, or —N(R)C(O)R. In some embodiments, R^{F} is —NR₂, —N(R)C(O)OR, —N(R)C(O)R, or —N(R)S(O)₂R.

In some embodiments, R^{F} is selected from the groups depicted in the compounds in Table 1.

As defined generally above, each instance of R^{H} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is a C₁₋₆ aliphatic chain. In some embodiments, R^{H} is phenyl. In some embodiments, R^{H} is naphthyl. In some embodiments, R^{H} is cubanyl. In some embodiments, R^{H} is adamantyl. In some embodiments, R^{H} is a 5-6 membered monocyclic heteroaryl ring having 1-4 heteroatoms independently selected from nitrogen, oxygen, and sulfur. In some embodiments, R^{H} is an 8-10 membered bicyclic heteroaryl ring having 1-4 heteroatoms independently selected from nitrogen, oxygen, and sulfur. In some embodiments, R^{H} is a 3-7 membered saturated or partially unsaturated monocyclic carbocyclic ring. In some embodiments, R^{H} is a 5-12 membered saturated or partially unsaturated bicyclic carbocyclic ring. In some embodiments, R^{H} is a 3-7 membered saturated or partially unsaturated monocyclic heterocyclic ring having 1-2 heteroatoms independently selected from nitrogen, oxygen, and sulfur. In some embodiments, R^{H} is a 7-12 membered saturated or partially unsaturated bicyclic heterocyclic ring having 1-4 heteroatoms independently selected from nitrogen, oxygen, and sulfur.

In some embodiments, R^{H} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{H} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{H} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{H} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{H} is phenyl or naphthyl. In some embodiments, R^{H} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{H} is a 3-7 membered saturated or partially unsaturated monocyclic carbocyclic ring or a 5-12 membered saturated or partially unsaturated bicyclic carbocyclic ring. In some embodiments, R^{H} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is phenyl or a 5-6 membered monocyclic heteroaryl ring having 1-4 heteroatoms independently selected from nitrogen, oxygen, and sulfur. In some embodiments, R^{H} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{H} is naphthyl or an 8-10 membered bicyclic heteroaryl ring having 1-4 heteroatoms independently selected from nitrogen, oxygen, and sulfur. In some embodiments, R^{H} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{H} is phenyl or a 3-7 membered saturated or partially unsaturated monocyclic carbocyclic ring. In some embodiments, R^{H} is naphthyl or a 5-12 membered saturated or partially unsaturated bicyclic carbocyclic ring. In some embodiments, R^{H} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{H} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{H} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{H} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{H} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{H} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{H} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{H} is a C₁₋₆ aliphatic chain, phenyl, or a 3-7 membered saturated or partially unsaturated monocyclic carbocyclic ring.

In some embodiments, R^{H} is selected from the groups depicted in the compounds in Table 1.

As defined generally above, each instance of R^{5C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{5C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{5C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{5C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{5C} is oxo. In some embodiments, R^{5C} is deuterium. In some embodiments, each instance of R^{5C} is independently halogen. In some embodiments, R^{5C} is —CN. In some embodiments, R^{5C} is —NO₂. In some embodiments, R^{5C} is —OR. In some embodiments, R^{5C} is —SR. In some embodiments, R^{5C} is —NR₂. In some embodiments, R^{5C} is —S(O)₂R. In some embodiments, R^{5C} is —S(O)₂NR₂. In some embodiments, R^{5C} is —S(O)₂F. In some embodiments, R^{5C} is —S(O)R. In some embodiments, R^{5C} is —S(O)NR₂. In some embodiments, R^{5C} is —S(O)(NR)R. In some embodiments, R^{5C} is —C(O)R. In some embodiments, R^{5C} is —C(O)OR. In some embodiments, R^{5C} is —C(O)NR₂. In some embodiments, R^{5C} is —C(O)N(R)OR. In some embodiments, R^{5C} is —OC(O)R. In some embodiments, R^{5C} is —OC(O)NR₂. In some embodiments, R^{5C} is —N(R)C(O)OR. In some embodiments, R^{5C} is —N(R)C(O)R. In some embodiments, R^{5C} is —N(R)C(O)NR₂. In some embodiments, R^{5C} is —N(R)C(NR)NR₂. In some embodiments, R^{5C} is —N(R)S(O)₂NR₂. In some embodiments, R^{5C} is —N(R)S(O)₂R. In some embodiments, R^{5C} is —P(O)R₂. In some embodiments, R^{5C} is —P(O)(R)OR. In some embodiments, R^{5C} is —B(OR)₂.

In some embodiments, each instance of R^{5C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{5C} is independently halogen, —CN, or —NO₂. In some embodiments, each instance of R^{5C} is independently —OR, —SR, or —NR₂. In some embodiments, each instance of R^{5C} is independently —S(O)₂R, —S(O)₂NR₂, —S(O)₂F, —S(O)R, —S(O)NR₂, or —S(O)(NR)R. In some embodiments, each instance of R^{5C} is independently —C(O)R, —C(O)OR, —C(O)NR₂, or —C(O)N(R)OR. In some embodiments, each instance of R^{5C} is independently —OC(O)R or —OC(O)NR₂. In some embodiments, each instance of R^{5C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{5C} is independently —P(O)R₂ or —P(O)(R)OR.

In some embodiments, each instance of R^{5C} is independently —OR, —OC(O)R, or —OC(O)NR₂. In some embodiments, each instance of R^{5C} is independently —SR, —S(O)₂R, —S(O)₂NR₂, —S(O)₂F, —S(O)R, —S(O)NR₂, or —S(O)(NR)R. In some embodiments, each instance of R^{5C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{5C} is independently —S(O)₂R, —S(O)₂NR₂, or —S(O)₂F. In some embodiments, each instance of R^{5C} is independently —S(O)R, —S(O)NR₂, or —S(O)(NR)R. In some embodiments, each instance of R^{5C} is independently —SR, —S(O)₂R, or —S(O)R. In some embodiments, each instance of R^{5C} is independently —S(O)₂NR₂, —S(O)NR₂, or —S(O)(NR)R. In some embodiments, each instance of R^{5C} is independently —S(O)₂NR₂ or —S(O)NR₂. In some embodiments, each instance of R^{5C} is independently —SR, —S(O)₂R, —S(O)₂NR₂, or —S(O)R.

In some embodiments, each instance of R^{5C} is independently —N(R)C(O)OR, —N(R)C(O)R, or —N(R)C(O)NR₂. In some embodiments, each instance of R^{5C} is independently —N(R)S(O)₂NR₂ or —N(R)S(O)₂R. In some embodiments, each instance of R^{5C} is independently —N(R)C(O)OR or —N(R)C(O)R. In some embodiments, each instance of R^{5C} is independently —N(R)C(O)NR₂ or —N(R)S(O)₂NR₂. In some embodiments, each instance of R^{5C} is independently —N(R)C(O)OR, —N(R)C(O)R, or —N(R)S(O)₂R.

In some embodiments, each instance of R^{5C} is independently —NR₂, —N(R)C(O)OR, —N(R)C(O)R, or —N(R)C(O)NR₂. In some embodiments, each instance of R^{5C} is independently —NR₂, —N(R)C(O)OR, or —N(R)C(O)R. In some embodiments, each instance of R^{5C} is independently —NR₂, —N(R)C(O)OR, —N(R)C(O)R, or —N(R)S(O)₂R.

In some embodiments, each instance of R^{5C} is independently an optionally substituted C₁₋₆ aliphatic. In some embodiments, each instance of R^{5C} is independently an optionally substituted phenyl. In some embodiments, each instance of R^{5C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{5C} is independently an optionally substituted 5-6 membered monocyclic heteroaryl ring having 1-4 heteroatoms independently selected from nitrogen, oxygen, and sulfur.

In some embodiments, each instance of R^{5C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{5C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{5C} is independently an optionally substituted C₁₋₆ aliphatic or an optionally substituted phenyl. In some embodiments, each instance of R^{5C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{5C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{5C} is independently a C₁₋₆ aliphatic. In some embodiments, R^{5C} is phenyl. In some embodiments, each instance of R^{5C} is independently a 3-7 membered saturated or partially unsaturated monocyclic heterocyclic ring having 1-2 heteroatoms independently selected from nitrogen, oxygen, and sulfur. In some embodiments, each instance of R^{5C} is independently a 5-6 membered monocyclic heteroaryl ring having 1-4 heteroatoms independently selected from nitrogen, oxygen, and sulfur.

In some embodiments, each instance of R^{5C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{5C} is independently phenyl or a 5-6 membered monocyclic heteroaryl ring having 1-4 heteroatoms independently selected from nitrogen, oxygen, and sulfur.

In some embodiments, each instance of R^{5C} is independently a C₁₋₆ aliphatic or phenyl. In some embodiments, each instance of R^{5C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{5C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and

In some embodiments, each instance of R^{5C} is independently halogen, —CN, —O-(optionally substituted C₁₋₆ aliphatic), or an optionally substituted C₁₋₆ aliphatic. In some embodiments, each instance of R^{5C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{5C} is independently halogen or C₁₋₃ aliphatic optionally substituted with 1-3 halogen. In some embodiments, each instance of R^{5C} is independently fluorine, chlorine, —CH₃, —CHF₂, or —CF₃.

In some embodiments, each instance of R^{5C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{5C} is independently selected from the groups depicted in the compounds in Table 1.

As defined generally above, each instance of R^{6C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{6C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{6C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{6C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{6C} is oxo. In some embodiments, R^{6C} is deuterium. In some embodiments, each instance of R^{6C} is independently halogen. In some embodiments, R^{6C} is —CN. In some embodiments, R^{6C} is —NO₂. In some embodiments, R^{6C} is —OR. In some embodiments, R^{6C} is —SR. In some embodiments, R^{6C} is —NR₂. In some embodiments, R^{6C} is —S(O)₂R. In some embodiments, R^{6C} is —S(O)₂NR₂. In some embodiments, R^{6C} is —S(O)₂F. In some embodiments, R^{6C} is —S(O)R. In some embodiments, R^{6C} is —S(O)NR₂. In some embodiments, R^{6C} is —S(O)(NR)R. In some embodiments, R^{6C} is —C(O)R. In some embodiments, R^{6C} is —C(O)OR. In some embodiments, R^{6C} is —C(O)NR₂. In some embodiments, R^{6C} is —C(O)N(R)OR. In some embodiments, R^{6C} is —OC(O)R. In some embodiments, R^{6C} is —OC(O)NR₂. In some embodiments, R^{6C} is —N(R)C(O)OR. In some embodiments, R^{6C} is —N(R)C(O)R. In some embodiments, R^{6C} is —N(R)C(O)NR₂. In some embodiments, R^{6C} is —N(R)C(NR)NR₂. In some embodiments, R^{6C} is —N(R)S(O)₂NR₂. In some embodiments, R^{6C} is —N(R)S(O)₂R. In some embodiments, R^{6C} is —P(O)R₂. In some embodiments, R^{6C} is —P(O)(R)OR. In some embodiments, R^{6C} is —B(OR)₂.

In some embodiments, each instance of R^{6C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{6C} is independently halogen, —CN, or —NO₂. In some embodiments, each instance of R^{6C} is independently —OR, —SR, or —NR₂. In some embodiments, each instance of R^{6C} is independently —S(O)₂R, —S(O)₂NR₂, —S(O)₂F, —S(O)R, —S(O)NR₂, or —S(O)(NR)R. In some embodiments, each instance of R^{6C} is independently —C(O)R, —C(O)OR, —C(O)NR₂, or —C(O)N(R)OR. In some embodiments, each instance of R^{6C} is independently —OC(O)R or —OC(O)NR₂. In some embodiments, each instance of R^{6C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{6C} is independently —P(O)R₂ or —P(O)(R)OR.

In some embodiments, each instance of R^{6C} is independently —OR, —OC(O)R, or —OC(O)NR₂. In some embodiments, each instance of R^{6C} is independently —SR, —S(O)₂R, —S(O)₂NR₂, —S(O)₂F, —S(O)R, —S(O)NR₂, or —S(O)(NR)R. In some embodiments, each instance of R^{6C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{6C} is independently —S(O)₂R, —S(O)₂NR₂, or —S(O)₂F. In some embodiments, each instance of R^{6C} is independently —S(O)R, —S(O)NR₂, or —S(O)(NR)R. In some embodiments, each instance of R^{6C} is independently —SR, —S(O)₂R, or —S(O)R. In some embodiments, each instance of R^{6C} is independently —S(O)₂NR₂, —S(O)NR₂, or —S(O)(NR)R. In some embodiments, each instance of R^{6C} is independently —S(O)₂NR₂ or —S(O)NR₂. In some embodiments, each instance of R^{6C} is independently —SR, —S(O)₂R, —S(O)₂NR₂, or —S(O)R.

In some embodiments, each instance of R^{6C} is independently —N(R)C(O)OR, —N(R)C(O)R, or —N(R)C(O)NR₂. In some embodiments, each instance of R^{6C} is independently —N(R)S(O)₂NR₂ or —N(R)S(O)₂R. In some embodiments, each instance of R^{6C} is independently —N(R)C(O)OR or —N(R)C(O)R. In some embodiments, each instance of R^{6C} is independently —N(R)C(O)NR₂ or —N(R)S(O)₂NR₂. In some embodiments, each instance of R^{6C} is independently —N(R)C(O)OR, —N(R)C(O)R, or —N(R)S(O)₂R.

In some embodiments, each instance of R^{6C} is independently —NR₂, —N(R)C(O)OR, —N(R)C(O)R, or —N(R)C(O)NR₂. In some embodiments, each instance of R^{6C} is independently —NR₂, —N(R)C(O)OR, or —N(R)C(O)R. In some embodiments, each instance of R^{6C} is independently —NR₂, —N(R)C(O)OR, —N(R)C(O)R, or —N(R)S(O)₂R.

In some embodiments, each instance of R^{6C} is independently an optionally substituted C₁₋₆ aliphatic. In some embodiments, each instance of R^{6C} is independently an optionally substituted phenyl. In some embodiments, each instance of R^{6C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{6C} is independently an optionally substituted 5-6 membered monocyclic heteroaryl ring having 1-4 heteroatoms independently selected from nitrogen, oxygen, and sulfur.

In some embodiments, each instance of R^{6C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{6C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{6C} is independently an optionally substituted C₁₋₆ aliphatic or an optionally substituted phenyl. In some embodiments, each instance of R^{6C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{6C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{6C} is independently a C₁₋₆ aliphatic. In some embodiments, R^{6C} is phenyl. In some embodiments, each instance of R^{6C} is independently a 3-7 membered saturated or partially unsaturated monocyclic heterocyclic ring having 1-2 heteroatoms independently selected from nitrogen, oxygen, and sulfur. In some embodiments, each instance of R^{6C} is independently a 5-6 membered monocyclic heteroaryl ring having 1-4 heteroatoms independently selected from nitrogen, oxygen, and sulfur.

In some embodiments, each instance of R^{6C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{6C} is independently phenyl or a 5-6 membered monocyclic heteroaryl ring having 1-4 heteroatoms independently selected from nitrogen, oxygen, and sulfur.

In some embodiments, each instance of R^{6C} is independently a C₁₋₆ aliphatic or phenyl. In some embodiments, each instance of R^{6C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{6C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{6C} is independently halogen, —CN, —O-(optionally substituted C₁₋₆ aliphatic), or an optionally substituted C₁₋₆ aliphatic. In some embodiments, each instance of R^{6C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{6C} is independently halogen or C₁₋₃ aliphatic optionally substituted with 1-3 halogen. In some embodiments, each instance of R^{6C} is independently fluorine, chlorine, —CH₃, —CHF₂, or —CF₃.

In some embodiments, each instance of R^{6C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{6C} is independently selected from the groups depicted in the compounds in Table 1.

As defined generally above, each instance of R^{EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{E1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{E1C} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{E1C} is independently deuterium, halogen, —CN, —OR, or —NR₂. In some embodiments, each instance of R^{E1C} is independently deuterium or halogen. In some embodiments, each instance of R^{E1C} is independently halogen.

In some embodiments, each instance of R^{E1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{E1C} is independently selected from the groups depicted in the compounds in Table 1.

As defined generally above, each instance of R^{GC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{GC} is independently C₁₋₆ aliphatic, oxo, deuterium, halogen, —CN, —OR, or —NR₂. In some embodiments, each instance of R^{GC} is independently C₁₋₃ aliphatic, oxo, deuterium, or halogen. In some embodiments, each instance of R^{GC} is oxo.

In some embodiments, each instance of R^{GC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{GC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{GC} is independently deuterium, halogen, —CN, —OR, or —NR₂. In some embodiments, each instance of R^{GC} is independently deuterium or halogen. In some embodiments, each instance of R^{GC} is independently halogen.

In some embodiments, each instance of R^{GC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{GC} is independently selected from the groups depicted in the compounds in Table 1.

As defined generally above, each instance of R^{Q1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Q1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Q1C} is oxo. In some embodiments, R^{Q1C} is deuterium. In some embodiments, each instance of R^{Q1C} is independently halogen. In some embodiments, R^{Q1C} is —CN. In some embodiments, R^{Q1C} is —NO₂. In some embodiments, R^{Q1C} is —OR. In some embodiments, R^{Q1C} is —SR. In some embodiments, R^{Q1C} is —NR₂. In some embodiments, R^{Q1C} is —S(O)₂R. In some embodiments, R^{Q1C} is —S(O)₂NR₂. In some embodiments, R^{Q1C} is —S(O)₂F. In some embodiments, R^{Q1C} is —S(O)R. In some embodiments, R^{Q1C} is —S(O)NR₂. In some embodiments, R^{Q1C} is —S(O)(NR)R. In some embodiments, R^{Q1C} is —C(O)R. In some embodiments, R^{Q1C} is —C(O)OR. In some embodiments, R^{Q1C} is —C(O)NR₂. In some embodiments, R^{Q1C} is —C(O)N(R)OR. In some embodiments, R^{Q1C} is —OC(O)R. In some embodiments, R^{Q1C} is —OC(O)NR₂. In some embodiments, R^{Q1C} is —N(R)C(O)OR. In some embodiments, R^{Q1C} is —N(R)C(O)R. In some embodiments, R^{Q1C} is —N(R)C(O)NR₂. In some embodiments, R^{Q1C} is —N(R)C(NR)NR₂. In some embodiments, R^{Q1C} is —N(R)S(O)₂NR₂. In some embodiments, R^{Q1C} is —N(R)S(O)₂R. In some embodiments, R^{Q1C} is —P(O)R₂. In some embodiments, R^{Q1C} is —P(O)(R)OR. In some embodiments, R^{Q1C} is —B(OR)₂.

In some embodiments, each instance of R^{Q1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Q1C} is independently halogen, —CN, or —NO₂. In some embodiments, each instance of R^{Q1C} is independently —OR, —SR, or —NR₂. In some embodiments, each instance of R^{Q1C} is independently —S(O)₂R, —S(O)₂NR₂, —S(O)₂F, —S(O)R, —S(O)NR₂, or —S(O)(NR)R. In some embodiments, each instance of R^{Q1C} is independently —C(O)R, —C(O)OR, —C(O)NR₂, or —C(O)N(R)OR. In some embodiments, each instance of R^{Q1C} is independently —OC(O)R or —OC(O)NR₂. In some embodiments, each instance of R^{Q1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Q1C} is independently —P(O)R₂ or —P(O)(R)OR.

In some embodiments, each instance of R^{Q1C} is independently —OR, —OC(O)R, or —OC(O)NR₂. In some embodiments, each instance of R^{Q1C} is independently —SR, —S(O)₂R, —S(O)₂NR₂, —S(O)₂F, —S(O)R, —S(O)NR₂, or —S(O)(NR)R. In some embodiments, each instance of R^{Q1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Q1C} is independently —S(O)₂R, —S(O)₂NR₂, or —S(O)₂F. In some embodiments, each instance of R^{Q1C} is independently —S(O)R, —S(O)NR₂, or —S(O)(NR)R. In some embodiments, each instance of R^{Q1C} is independently —SR, —S(O)₂R, or —S(O)R. In some embodiments, each instance of R^{Q1C} is independently —S(O)₂NR₂, —S(O)NR₂, or —S(O)(NR)R. In some embodiments, each instance of R^{Q1C} is independently —S(O)₂NR₂ or —S(O)NR₂. In some embodiments, each instance of R^{Q1C} is independently —SR, —S(O)₂R, —S(O)₂NR₂, or —S(O)R.

In some embodiments, each instance of R^{Q1C} is independently —N(R)C(O)OR, —N(R)C(O)R, or —N(R)C(O)NR₂. In some embodiments, each instance of R^{Q1C} is independently —N(R)S(O)₂NR₂ or —N(R)S(O)₂R. In some embodiments, each instance of R^{Q1C} is independently —N(R)C(O)OR or —N(R)C(O)R. In some embodiments, each instance of R^{Q1C} is independently —N(R)C(O)NR₂ or —N(R)S(O)₂NR₂. In some embodiments, each instance of R^{Q1C} is independently —N(R)C(O)OR, —N(R)C(O)R, or —N(R)S(O)₂R.

In some embodiments, each instance of R^{Q1C} is independently —NR₂, —N(R)C(O)OR, —N(R)C(O)R, or —N(R)C(O)NR₂. In some embodiments, each instance of R^{Q1C} is independently —NR₂, —N(R)C(O)OR, or —N(R)C(O)R. In some embodiments, each instance of R^{Q1C} is independently —NR₂, —N(R)C(O)OR, —N(R)C(O)R, or —N(R)S(O)₂R.

In some embodiments, each instance of R^{Q1C} is independently an optionally substituted C₁₋₆ aliphatic. In some embodiments, each instance of R^{Q1C} is independently an optionally substituted phenyl. In some embodiments, each instance of R^{Q1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Q1C} is independently an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Q1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted C₁₋₆ aliphatic or an optionally substituted phenyl. In some embodiments, each instance of R^{Q1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic. In some embodiments, R^{Q1C} is phenyl. In some embodiments, each instance of R^{Q1C} is independently a 3-7 membered saturated or partially unsaturated monocyclic heterocyclic ring having 1-2 heteroatoms independently selected from nitrogen, oxygen, and sulfur. In some embodiments, each instance of R^{Q1C} is independently a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Q1C} is independently phenyl or a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic or phenyl. In some embodiments, each instance of R^{Q1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q1C} is independently selected from the groups depicted in the compounds in Table 1.

As defined generally above, each instance of R^{Y1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Y1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Y1C} is oxo. In some embodiments, R^{Y1C} is deuterium. In some embodiments, each instance of R^{Y1C} is independently halogen. In some embodiments, R^{Y1C} is —CN. In some embodiments, R^{Y1C} is —NO₂. In some embodiments, R^{Y1C} is —OR. In some embodiments, R^{Y1C} is —SR. In some embodiments, R^{Y1C} is —NR₂. In some embodiments, R^{Y1C} is —S(O)₂R. In some embodiments, R^{Y1C} is —S(O)₂NR₂. In some embodiments, R^{Y1C} is —S(O)₂F. In some embodiments, R^{Y1C} is —S(O)R. In some embodiments, R^{Y1C} is —S(O)NR₂. In some embodiments, R^{Y1C} is —S(O)(NR)R. In some embodiments, R^{Y1C} is —C(O)R. In some embodiments, R^{Y1C} is —C(O)OR. In some embodiments, R^{Y1C} is —C(O)NR₂. In some embodiments, R^{Y1C} is —C(O)N(R)OR. In some embodiments, R^{Y1C} is —OC(O)R. In some embodiments, R^{Y1C} is —OC(O)NR₂. In some embodiments, R^{Y1C} is —N(R)C(O)OR. In some embodiments, R^{Y1C} is —N(R)C(O)R. In some embodiments, R^{Y1C} is —N(R)C(O)NR₂. In some embodiments, R^{Y1C} is —N(R)C(NR)NR₂. In some embodiments, R^{Y1C} is —N(R)S(O)₂NR₂. In some embodiments, R^{Y1C} is —N(R)S(O)₂R. In some embodiments, R^{Y1C} is —P(O)R₂. In some embodiments, R^{Y1C} is —P(O)(R)OR. In some embodiments, R^{Y1C} is —B(OR)₂.

In some embodiments, each instance of R^{Y1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Y1C} is independently halogen, —CN, or —NO₂. In some embodiments, each instance of R^{Y1C} is independently —OR, —SR, or —NR₂. In some embodiments, each instance of R^{Y1C} is independently —S(O)₂R, —S(O)₂NR₂, —S(O)₂F, —S(O)R, —S(O)NR₂, or —S(O)(NR)R. In some embodiments, each instance of R^{Y1C} is independently —C(O)R, —C(O)OR, —C(O)NR₂, or —C(O)N(R)OR. In some embodiments, each instance of R^{Y1C} is independently —OC(O)R or —OC(O)NR₂. In some embodiments, each instance of R^{Y1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Y1C} is independently —P(O)R₂ or —P(O)(R)OR.

In some embodiments, each instance of R^{Y1C} is independently —OR, —OC(O)R, or —OC(O)NR₂. In some embodiments, each instance of R^{Y1C} is independently —SR, —S(O)₂R, —S(O)₂NR₂, —S(O)₂F, —S(O)R, —S(O)NR₂, or —S(O)(NR)R. In some embodiments, each instance of R^{Y1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Y1C} is independently —S(O)₂R, —S(O)₂NR₂, or —S(O)₂F. In some embodiments, each instance of R^{Y1C} is independently —S(O)R, —S(O)NR₂, or —S(O)(NR)R. In some embodiments, each instance of R^{Y1C} is independently —SR, —S(O)₂R, or —S(O)R. In some embodiments, each instance of R^{Y1C} is independently —S(O)₂NR₂, —S(O)NR₂, or —S(O)(NR)R. In some embodiments, each instance of R^{Y1C} is independently —S(O)₂NR₂ or —S(O)NR₂. In some embodiments, each instance of R^{Y1C} is independently —SR, —S(O)₂R, —S(O)₂NR₂, or —S(O)R.

In some embodiments, each instance of R^{Y1C} is independently —N(R)C(O)OR, —N(R)C(O)R, or —N(R)C(O)NR₂. In some embodiments, each instance of R^{Y1C} is independently —N(R)S(O)₂NR₂ or —N(R)S(O)₂R. In some embodiments, each instance of R^{Y1c} is independently —N(R)C(O)OR or —N(R)C(O)R. In some embodiments, each instance of R^{Y1c} is independently —N(R)C(O)NR₂ or —N(R)S(O)₂NR₂. In some embodiments, each instance of R^{Y1c} is independently —N(R)C(O)OR, —N(R)C(O)R, or —N(R)S(O)₂R.

In some embodiments, each instance of R^{Y1c} is independently —NR₂, —N(R)C(O)OR, —N(R)C(O)R, or —N(R)C(O)NR₂. In some embodiments, each instance of R^{Y1c} is independently —NR₂, —N(R)C(O)OR, or —N(R)C(O)R. In some embodiments, each instance of R^{Y1c} is independently —NR₂, —N(R)C(O)OR, —N(R)C(O)R, or —N(R)S(O)₂R.

In some embodiments, each instance of R^{Y1c} is independently an optionally substituted C₁₋₆ aliphatic. In some embodiments, each instance of R^{Y1c} is independently an optionally substituted phenyl. In some embodiments, each instance of R^{Y1c} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Y1c} is independently an optionally substituted 5-6 membered monocyclic heteroaryl ring having 1-4 heteroatoms independently selected from nitrogen, oxygen, and sulfur.

In some embodiments, each instance of R^{Y1c} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Y1c} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Y1c} is independently an optionally substituted C₁₋₆ aliphatic or an optionally substituted phenyl. In some embodiments, each instance of R^{Y1c} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Y1c} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y1C} is independently a C₁₋₆ aliphatic. In some embodiments, R^{Y1C} is phenyl. In some embodiments, each instance of R^{Y1C} is independently a 3-7 membered saturated or partially unsaturated monocyclic heterocyclic ring having 1-2 heteroatoms independently selected from nitrogen, oxygen, and sulfur. In some embodiments, each instance of R^{Y1C} is independently a 5-6 membered monocyclic heteroaryl ring having 1-4 heteroatoms independently selected from nitrogen, oxygen, and sulfur.

In some embodiments, each instance of R^{Y1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Y1C} is independently phenyl or a 5-6 membered monocyclic heteroaryl ring having 1-4 heteroatoms independently selected from nitrogen, oxygen, and sulfur.

In some embodiments, each instance of R^{Y1C} is independently a C₁₋₆ aliphatic or phenyl. In some embodiments, each instance of R^{Y1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Y1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Y1C} is independently selected from the groups depicted in the compounds in Table 1.

As defined generally above, each instance of R^{Y2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Y2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Y2C} is oxo. In some embodiments, R^{Y2C} is deuterium. In some embodiments, each instance of R^{Y2C} is independently halogen. In some embodiments, R^{Y2C} is —CN. In some embodiments, R^{Y2C} is —NO₂. In some embodiments, R^{Y2C} is —OR. In some embodiments, R^{Y2C} is —SR. In some embodiments, R^{Y2C} is —NR₂. In some embodiments, R^{Y2C} is —S(O)₂R. In some embodiments, R^{Y2C} is —S(O)₂NR₂. In some embodiments, R^{Y2C} is —S(O)₂F. In some embodiments, R^{Y2C} is —S(O)R. In some embodiments, R^{Y2C} is —S(O)NR₂. In some embodiments, R^{Y2C} is —S(O)(NR)R. In some embodiments, R^{Y2C} is —C(O)R. In some embodiments, R^{Y2C} is —C(O)OR. In some embodiments, R^{Y2C} is —C(O)NR₂. In some embodiments, R^{Y2C} is —C(O)N(R)OR. In some embodiments, R^{Y2C} is —OC(O)R. In some embodiments, R^{Y2C} is —OC(O)NR₂. In some embodiments, R^{Y2C} is —N(R)C(O)OR. In some embodiments, R^{Y2C} is —N(R)C(O)R. In some embodiments, R^{Y2C} is —N(R)C(O)NR₂. In some embodiments, R^{Y2C} is —N(R)C(NR)NR₂. In some embodiments, R^{Y2C} is —N(R)S(O)₂NR₂. In some embodiments, R^{Y2C} is —N(R)S(O)₂R. In some embodiments, R^{Y2C} is —P(O)R₂. In some embodiments, R^{Y2C} is —P(O)(R)OR. In some embodiments, R^{Y2C} is —B(OR)₂.

In some embodiments, each instance of R^{Y2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Y2C} is independently halogen, —CN, or —NO₂. In some embodiments, each instance of R^{Y2C} is independently —OR, —SR, or —NR₂. In some embodiments, each instance of R^{Y2C} is independently —S(O)₂R, —S(O)₂NR₂, —S(O)₂F, —S(O)R, —S(O)NR₂, or —S(O)(NR)R. In some embodiments, each instance of R^{Y2C} is independently —C(O)R, —C(O)OR, —C(O)NR₂, or —C(O)N(R)OR. In some embodiments, each instance of R^{Y2C} is independently —OC(O)R or —OC(O)NR₂. In some embodiments, each instance of R^{Y2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Y2C} is independently —P(O)R₂ or —P(O)(R)OR.

In some embodiments, each instance of R^{Y2C} is independently —OR, —OC(O)R, or —OC(O)NR₂. In some embodiments, each instance of R^{Y2C} is independently —SR, —S(O)₂R, —S(O)₂NR₂, —S(O)₂F, —S(O)R, —S(O)NR₂, or —S(O)(NR)R. In some embodiments, each instance of R^{Y2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Y2C} is independently —S(O)₂R, —S(O)₂NR₂, or —S(O)₂F. In some embodiments, each instance of R^{Y2C} is independently —S(O)R, —S(O)NR₂, or —S(O)(NR)R. In some embodiments, each instance of R^{Y2C} is independently —SR, —S(O)₂R, or —S(O)R. In some embodiments, each instance of R^{Y2C} is independently —S(O)₂NR₂, —S(O)NR₂, or —S(O)(NR)R. In some embodiments, each instance of R^{Y2C} is independently —S(O)₂NR₂ or —S(O)NR₂. In some embodiments, each instance of R^{Y2C} is independently —SR, —S(O)₂R, —S(O)₂NR₂, or —S(O)R.

In some embodiments, each instance of R^{Y2C} is independently —N(R)C(O)OR, —N(R)C(O)R, or —N(R)C(O)NR₂. In some embodiments, each instance of R^{Y2C} is independently —N(R)S(O)₂NR₂ or —N(R)S(O)₂R. In some embodiments, each instance of R^{Y2C} is independently —N(R)C(O)OR or —N(R)C(O)R. In some embodiments, each instance of R^{Y2C} is independently —N(R)C(O)NR₂ or —N(R)S(O)₂NR₂. In some embodiments, each instance of R^{Y2C} is independently —N(R)C(O)OR, —N(R)C(O)R, or —N(R)S(O)₂R.

In some embodiments, each instance of R^{Y2C} is independently —NR₂, —N(R)C(O)OR, —N(R)C(O)R, or —N(R)C(O)NR₂. In some embodiments, each instance of R^{Y2C} is independently —NR₂, —N(R)C(O)OR, or —N(R)C(O)R. In some embodiments, each instance of R^{Y2C} is independently —NR₂, —N(R)C(O)OR, —N(R)C(O)R, or —N(R)S(O)₂R.

In some embodiments, each instance of R^{Y2C} is independently an optionally substituted C₁₋₆ aliphatic. In some embodiments, each instance of R^{Y2C} is independently an optionally substituted phenyl. In some embodiments, each instance of R^{Y2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Y2C} is independently an optionally substituted 5-6 membered monocyclic heteroaryl ring having 1-4 heteroatoms independently selected from nitrogen, oxygen, and sulfur.

In some embodiments, each instance of R^{Y2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Y2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Y2C} is independently an optionally substituted C₁₋₆ aliphatic or an optionally substituted phenyl. In some embodiments, each instance of R^{Y2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Y2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Y2C} is independently a C₁₋₆ aliphatic. In some embodiments, R^{Y2C} is phenyl. In some embodiments, each instance of R^{Y2C} is independently a 3-7 membered saturated or partially unsaturated monocyclic heterocyclic ring having 1-2 heteroatoms independently selected from nitrogen, oxygen, and sulfur. In some embodiments, each instance of R^{Y2C} is independently a 5-6 membered monocyclic heteroaryl ring having 1-4 heteroatoms independently selected from nitrogen, oxygen, and sulfur.

In some embodiments, each instance of R^{Y2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Y2C} is independently phenyl or a 5-6 membered monocyclic heteroaryl ring having 1-4 heteroatoms independently selected from nitrogen, oxygen, and sulfur.

In some embodiments, each instance of R^{Y2C} is independently a C₁₋₆ aliphatic or phenyl. In some embodiments, each instance of R^{Y2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Y2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Y2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{Y2C} is independently halogen, —CN, —OH, —O-(optionally substituted C₁₋₃ aliphatic), or an optionally substituted C₁₋₃ aliphatic. In some embodiments, each instance of R^{Y2C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{Y2C} is independently fluorine, chlorine, —OH, —OCH₃, —OCF₃, —CH₃, —CHF₂, or —CF₃. In some embodiments, each instance of R^{Y2C} is independently fluorine or —OH.

In some embodiments, each instance of R^{Y2C} is independently oxo, deuterium, halogen, —CN, —OH, —O-(optionally substituted C₁₋₃ aliphatic), or an optionally substituted C₁₋₃ aliphatic. In some embodiments, each instance of R^{Y2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{Y2C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{Y2C} is independently oxo, deuterium, fluorine, chlorine, —CN, —OH, —OCH₃, —OCF₃, —CH₃, —CHF₂, or —CF₃. In some embodiments, each instance of R^{Y2C} is independently oxo, deuterium, —CN, fluorine, or —OH. In some embodiments, each instance of R^{Y2C} is independently oxo, deuterium, —CN, —CH₃, or —CHF₂. In some embodiments, each instance of R^{Y2C} is independently deuterium, —CN, —CH₃, or —CHF₂.

In some embodiments, each instance of R^{Y2C} is independently oxo, halogen, —CN, —OH, —O-(optionally substituted C₁₋₃ aliphatic), or an optionally substituted C₁₋₃ aliphatic. In some embodiments, each instance of R^{Y2C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{Y2C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{Y2C} is independently oxo, fluorine, chlorine, —CN, —OH, —OCH₃, —OCF₃, —CH₃, —CHF₂, or —CF₃. In some embodiments, each instance of R^{Y2C} is independently oxo, —CN, fluorine, or —OH. In some embodiments, each instance of R^{Y2C} is independently oxo, —CN, —CH₃, or —CHF₂. In some embodiments, each instance of R^{Y2C} is independently —CN, —CH₃, or —CHF₂.

In some embodiments, each instance of R^{Y2C} is independently selected from the groups depicted in the compounds in Table 1.

As defined generally above, each instance of R^{L2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{L2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{L2C} is oxo. In some embodiments, R^{L2C} is deuterium. In some embodiments, each instance of R^{L2C} is independently halogen. In some embodiments, R^{L2C} is —CN. In some embodiments, R^{L2C} is —NO₂. In some embodiments, R^{L2C} is —OR. In some embodiments, R^{L2C} is —SR. In some embodiments, R^{L2C} is —NR₂. In some embodiments, R^{L2C} is —S(O)₂R. In some embodiments, R^{L2C} is —S(O)₂NR₂. In some embodiments, R^{L2C} is —S(O)₂F. In some embodiments, R^{L2C} is —S(O)R. In some embodiments, R^{L2C} is —S(O)NR₂. In some embodiments, R^{L2C} is —S(O)(NR)R. In some embodiments, R^{L2C} is —C(O)R. In some embodiments, R^{L2C} is —C(O)OR. In some embodiments, R^{L2C} is —C(O)NR₂. In some embodiments, R^{L2C} is —C(O)N(R)OR. In some embodiments, R^{L2C} is —OC(O)R. In some embodiments, R^{L2C} is —OC(O)NR₂. In some embodiments, R^{L2C} is —N(R)C(O)OR. In some embodiments, R^{L2C} is —N(R)C(O)R. In some embodiments, R^{L2C} is —N(R)C(O)NR₂. In some embodiments, R^{L2C} is —N(R)C(NR)NR₂. In some embodiments, R^{L2C} is —N(R)S(O)₂NR₂. In some embodiments, R^{L2C} is —N(R)S(O)₂R. In some embodiments, R^{L2C} is —P(O)R₂. In some embodiments, R^{L2C} is —P(O)(R)OR. In some embodiments, R^{L2C} is —B(OR)₂.

In some embodiments, each instance of R^{L2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{L2C} is independently halogen, —CN, or —NO₂. In some embodiments, each instance of R^{L2C} is independently —OR, —SR, or —NR₂. In some embodiments, each instance of R^{L2C} is independently —S(O)₂R, —S(O)₂NR₂, —S(O)₂F, —S(O)R, —S(O)NR₂, or —S(O)(NR)R. In some embodiments, each instance of R^{L2C} is independently —C(O)R, —C(O)OR, —C(O)NR₂, or —C(O)N(R)OR. In some embodiments, each instance of R^{L2C} is independently —OC(O)R or —OC(O)NR₂. In some embodiments, each instance of R^{L2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{L2C} is independently —P(O)R₂ or —P(O)(R)OR.

In some embodiments, each instance of R^{L2C} is independently —OR, —OC(O)R, or —OC(O)NR₂. In some embodiments, each instance of R^{L2C} is independently —SR, —S(O)₂R, —S(O)₂NR₂, —S(O)₂F, —S(O)R, —S(O)NR₂, or —S(O)(NR)R. In some embodiments, each instance of R^{L2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{L2C} is independently —S(O)₂R, —S(O)₂NR₂, or —S(O)₂F. In some embodiments, each instance of R^{L2C} is independently —S(O)R, —S(O)NR₂, or —S(O)(NR)R. In some embodiments, each instance of R^{L2C} is independently —SR, —S(O)₂R, or —S(O)R. In some embodiments, each instance of R^{L2C} is independently —S(O)₂NR₂, —S(O)NR₂, or —S(O)(NR)R. In some embodiments, each instance of R^{L2C} is independently —S(O)₂NR₂ or —S(O)NR₂. In some embodiments, each instance of R^{L2C} is independently —SR, —S(O)₂R, —S(O)₂NR₂, or —S(O)R.

In some embodiments, each instance of R^{L2C} is independently —N(R)C(O)OR, —N(R)C(O)R, or —N(R)C(O)NR₂. In some embodiments, each instance of R^{L2C} is independently —N(R)S(O)₂NR₂ or —N(R)S(O)₂R. In some embodiments, each instance of R^{L2C} is independently —N(R)C(O)OR or —N(R)C(O)R. In some embodiments, each instance of R^{L2C} is independently —N(R)C(O)NR₂ or —N(R)S(O)₂NR₂. In some embodiments, each instance of R^{L2C} is independently —N(R)C(O)OR, —N(R)C(O)R, or —N(R)S(O)₂R.

In some embodiments, each instance of R^{L2C} is independently —NR₂, —N(R)C(O)OR, —N(R)C(O)R, or —N(R)C(O)NR₂. In some embodiments, each instance of R^{L2C} is independently —NR₂, —N(R)C(O)OR, or —N(R)C(O)R. In some embodiments, each instance of R^{L2C} is independently —NR₂, —N(R)C(O)OR, —N(R)C(O)R, or —N(R)S(O)₂R.

In some embodiments, each instance of R^{L2C} is independently an optionally substituted C₁₋₆ aliphatic. In some embodiments, each instance of R^{L2C} is independently an optionally substituted phenyl. In some embodiments, each instance of R^{L2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{L2C} is independently an optionally substituted 5-6 membered monocyclic heteroaryl ring having 1-4 heteroatoms independently selected from nitrogen, oxygen, and sulfur.

In some embodiments, each instance of R^{L2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{L2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{L2C} is independently an optionally substituted C₁₋₆ aliphatic or an optionally substituted phenyl. In some embodiments, each instance of R^{L2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{L2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L2C} is independently a C₁₋₆ aliphatic. In some embodiments, R^{L2C} is phenyl. In some embodiments, each instance of R^{L2C} is independently a 3-7 membered saturated or partially unsaturated monocyclic heterocyclic ring having 1-2 heteroatoms independently selected from nitrogen, oxygen, and sulfur. In some embodiments, each instance of R^{L2C} is independently a 5-6 membered monocyclic heteroaryl ring having 1-4 heteroatoms independently selected from nitrogen, oxygen, and sulfur.

In some embodiments, each instance of R^{L2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{L2C} is independently phenyl or a 5-6 membered monocyclic heteroaryl ring having 1-4 heteroatoms independently selected from nitrogen, oxygen, and sulfur.

In some embodiments, each instance of R^{L2C} is independently a C₁₋₆ aliphatic or phenyl. In some embodiments, each instance of R^{L2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L2C} is independently selected from the groups depicted in the compounds in Table 1.

As defined generally above, each instance of R^{E1EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{E1EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{E1EC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{EEC} is independently deuterium, halogen, —CN, —OR, or —NR₂. In some embodiments, each instance of R^{E1EC} is independently deuterium or halogen. In some embodiments, each instance of R^{E1EC} is independently halogen.

In some embodiments, each instance of R^{E1EC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{E1EC} is independently selected from the groups depicted in the compounds in Table 1.

As defined generally above, each instance of R^{Q5C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q5C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q5C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Q5C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Q5C} is oxo. In some embodiments, R^{Q5C} is deuterium. In some embodiments, each instance of R^{Q5C} is independently halogen. In some embodiments, R^{Q5C} is —CN. In some embodiments, R^{Q5C} is —NO₂. In some embodiments, R^{Q5C} is —OR. In some embodiments, R^{Q5C} is —SR. In some embodiments, R^{Q5C} is —NR₂. In some embodiments, R^{Q5C} is —S(O)₂R. In some embodiments, R^{Q5C} is —S(O)₂NR₂. In some embodiments, R^{Q5C} is —S(O)₂F. In some embodiments, R^{Q5C} is —S(O)R. In some embodiments, R^{Q5C} is —S(O)NR₂. In some embodiments, R^{Q5C} is —S(O)(NR)R. In some embodiments, R^{Q5C} is —C(O)R. In some embodiments, R^{Q5C} is —C(O)OR. In some embodiments, R^{Q5C} is —C(O)NR₂. In some embodiments, R^{Q5C} is —C(O)N(R)OR. In some embodiments, R^{Q5C} is —OC(O)R. In some embodiments, R^{Q5C} is —OC(O)NR₂. In some embodiments, R^{Q5C} is —N(R)C(O)OR. In some embodiments, R^{Q5C} is —N(R)C(O)R. In some embodiments, R^{Q5C} is —N(R)C(O)NR₂. In some embodiments, R^{Q5C} is —N(R)C(NR)NR₂. In some embodiments, R^{Q5C} is —N(R)S(O)₂NR₂. In some embodiments, R^{Q5C} is —N(R)S(O)₂R. In some embodiments, R^{Q5C} is —P(O)R₂. In some embodiments, R^{Q5C} is —P(O)(R)OR. In some embodiments, R^{Q5C} is —B(OR)₂.

In some embodiments, each instance of R^{Q5C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Q5C} is independently halogen, —CN, or —NO₂. In some embodiments, each instance of R^{Q5C} is independently —OR, —SR, or —NR₂. In some embodiments, each instance of R^{Q5C} is independently —S(O)₂R, —S(O)₂NR₂, —S(O)₂F, —S(O)R, —S(O)NR₂, or —S(O)(NR)R. In some embodiments, each instance of R^{Q5C} is independently —C(O)R, —C(O)OR, —C(O)NR₂, or —C(O)N(R)OR. In some embodiments, each instance of R^{Q5C} is independently —OC(O)R or —OC(O)NR₂. In some embodiments, each instance of R^{Q5C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Q5C} is independently —P(O)R₂ or —P(O)(R)OR.

In some embodiments, each instance of R^{Q5C} is independently —OR, —OC(O)R, or —OC(O)NR₂. In some embodiments, each instance of R^{Q5C} is independently —SR, —S(O)₂R, —S(O)₂NR₂, —S(O)₂F, —S(O)R, —S(O)NR₂, or —S(O)(NR)R. In some embodiments, each instance of R^{Q5C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Q5C} is independently —S(O)₂R, —S(O)₂NR₂, or —S(O)₂F. In some embodiments, each instance of R^{Q5C} is independently —S(O)R, —S(O)NR₂, or —S(O)(NR)R. In some embodiments, each instance of R^{Q5C} is independently —SR, —S(O)₂R, or —S(O)R. In some embodiments, each instance of R^{Q5C} is independently —S(O)₂NR₂, —S(O)NR₂, or —S(O)(NR)R. In some embodiments, each instance of R^{Q5C} is independently —S(O)₂NR₂ or —S(O)NR₂. In some embodiments, each instance of R^{Q5C} is independently —SR, —S(O)₂R, —S(O)₂NR₂, or —S(O)R.

In some embodiments, each instance of R^{Q5C} is independently —N(R)C(O)OR, —N(R)C(O)R, or —N(R)C(O)NR₂. In some embodiments, each instance of R^{Q5C} is independently —N(R)S(O)₂NR₂ or —N(R)S(O)₂R. In some embodiments, each instance of R^{Q5C} is independently —N(R)C(O)OR or —N(R)C(O)R. In some embodiments, each instance of R^{Q5C} is independently —N(R)C(O)NR₂ or —N(R)S(O)₂NR₂. In some embodiments, each instance of R^{Q5C} is independently —N(R)C(O)OR, —N(R)C(O)R, or —N(R)S(O)₂R.

In some embodiments, each instance of R^{Q5C} is independently —NR₂, —N(R)C(O)OR, —N(R)C(O)R, or —N(R)C(O)NR₂. In some embodiments, each instance of R^{Q5C} is independently —NR₂, —N(R)C(O)OR, or —N(R)C(O)R. In some embodiments, each instance of R^{Q5C} is independently —NR₂, —N(R)C(O)OR, —N(R)C(O)R, or —N(R)S(O)₂R.

In some embodiments, each instance of R^{Q5C} is independently an optionally substituted C₁₋₆ aliphatic. In some embodiments, each instance of R^{Q5C} is independently an optionally substituted phenyl. In some embodiments, each instance of R^{Q5C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Q5C} is independently an optionally substituted 5-6 membered monocyclic heteroaryl ring having 1-4 heteroatoms independently selected from nitrogen, oxygen, and sulfur.

In some embodiments, each instance of R^{Q5C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Q5C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Q5C} is independently an optionally substituted C₁₋₆ aliphatic or an optionally substituted phenyl. In some embodiments, each instance of R^{Q5C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Q5C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q5C} is independently a C₁₋₆ aliphatic. In some embodiments, R^{Q5C} is phenyl. In some embodiments, each instance of R^{Q5C} is independently a 3-7 membered saturated or partially unsaturated monocyclic heterocyclic ring having 1-2 heteroatoms independently selected from nitrogen, oxygen, and sulfur. In some embodiments, each instance of R^{Q5C} is independently a 5-6 membered monocyclic heteroaryl ring having 1-4 heteroatoms independently selected from nitrogen, oxygen, and sulfur.

In some embodiments, each instance of R^{Q5C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Q5C} is independently phenyl or a 5-6 membered monocyclic heteroaryl ring having 1-4 heteroatoms independently selected from nitrogen, oxygen, and sulfur.

In some embodiments, each instance of R^{Q5C} is independently a C₁₋₆ aliphatic or phenyl. In some embodiments, each instance of R^{Q5C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q5C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q5C} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n² is 0, 1, 2, 3, 4, or 5. In some embodiments, n² is 0. In some embodiments, n² is 1. In some embodiments, n² is 2. In some embodiments, n² is 3. In some embodiments, n² is 4. In some embodiments, n² is 5. In some embodiments, n² is 0 or 1. In some embodiments, n² is 0, 1, or 2. In some embodiments, n² is 0, 1, 2, or 3. In some embodiments, n² is 0, 1, 2, 3, or 4. In some embodiments, n² is 1 or 2. In some embodiments, n² is 1, 2, or 3. In some embodiments, n² is 1, 2, 3, or 4. In some embodiments, n² is 1, 2, 3, 4, or 5. In some embodiments, n² is 2 or 3. In some embodiments, n² is 2, 3, or 4. In some embodiments, n² is 2, 3, 4, or 5. In some embodiments, n² is 3 or 4. In some embodiments, n² is 3, 4, or 5. In some embodiments, n² is 4 or 5. In some embodiments, n² is selected from the values represented in the compounds in Table 1.

As defined generally above, p² is 0, 1, 2, 3, 4, or 5. In some embodiments, p² is 0. In some embodiments, p² is 1. In some embodiments, p² is 2. In some embodiments, p² is 3. In some embodiments, p² is 4. In some embodiments, p² is 5. In some embodiments, p² is 0 or 1. In some embodiments, p² is 0, 1, or 2. In some embodiments, p² is 0, 1, 2, or 3. In some embodiments, p² is 0, 1, 2, 3, or 4. In some embodiments, p² is 1 or 2. In some embodiments, p² is 1, 2, or 3. In some embodiments, p² is 1, 2, 3, or 4. In some embodiments, p² is 1, 2, 3, 4, or 5. In some embodiments, p² is 2 or 3. In some embodiments, p² is 2, 3, or 4. In some embodiments, p² is 2, 3, 4, or 5. In some embodiments, p² is 3 or 4. In some embodiments, p² is 3, 4, or 5. In some embodiments, p² is 4 or 5. In some embodiments, p² is selected from the values represented in the compounds in Table 1.

As defined generally above, v¹ is 0, 1, 2, 3, 4, or 5. In some embodiments, v¹ is 0. In some embodiments, v¹ is 1. In some embodiments, v¹ is 2. In some embodiments, v¹ is 3. In some embodiments, v¹ is 4. In some embodiments, v¹ is 5. In some embodiments, v¹ is 0 or 1. In some embodiments, v¹ is 0, 1, or 2. In some embodiments, v¹ is 0, 1, 2, or 3. In some embodiments, v¹ is 0, 1, 2, 3, or 4. In some embodiments, v¹ is 1 or 2. In some embodiments, v¹ is 1, 2, or 3. In some embodiments, v¹ is 1, 2, 3, or 4. In some embodiments, v¹ is 1, 2, 3, 4, or 5. In some embodiments, v¹ is 2 or 3. In some embodiments, v¹ is 2, 3, or 4. In some embodiments, v¹ is 2, 3, 4, or 5. In some embodiments, v¹ is 3 or 4. In some embodiments, v¹ is 3, 4, or 5. In some embodiments, v¹ is 4 or 5. In some embodiments, v¹ is selected from the values represented in the compounds in Table 1.

As defined generally above, v² is 0, 1, 2, 3, 4, or 5. In some embodiments, v² is 0. In some embodiments, v² is 1. In some embodiments, v² is 2. In some embodiments, v² is 3. In some embodiments, v² is 4. In some embodiments, v² is 5. In some embodiments, v² is 0 or 1. In some embodiments, v² is 0, 1, or 2. In some embodiments, v² is 0, 1, 2, or 3. In some embodiments, v² is 0, 1, 2, 3, or 4. In some embodiments, v² is 1 or 2. In some embodiments, v² is 1, 2, or 3. In some embodiments, v² is 1, 2, 3, or 4. In some embodiments, v² is 1, 2, 3, 4, or 5. In some embodiments, v² is 2 or 3. In some embodiments, v² is 2, 3, or 4. In some embodiments, v² is 2, 3, 4, or 5. In some embodiments, v² is 3 or 4. In some embodiments, v² is 3, 4, or 5. In some embodiments, v² is 4 or 5. In some embodiments, v² is selected from the values represented in the compounds in Table 1.

As defined generally above, v³ is 0, 1, 2, 3, 4, or 5. In some embodiments, v³ is 0. In some embodiments, v³ is 1. In some embodiments, v³ is 2. In some embodiments, v³ is 3. In some embodiments, v³ is 4. In some embodiments, v³ is 5. In some embodiments, v³ is 0 or 1. In some embodiments, v³ is 0, 1, or 2. In some embodiments, v³ is 0, 1, 2, or 3. In some embodiments, v³ is 0, 1, 2, 3, or 4. In some embodiments, v³ is 1 or 2. In some embodiments, v³ is 1, 2, or 3. In some embodiments, v³ is 1, 2, 3, or 4. In some embodiments, v³ is 1, 2, 3, 4, or 5. In some embodiments, v³ is 2 or 3. In some embodiments, v³ is 2, 3, or 4. In some embodiments, v³ is 2, 3, 4, or 5. In some embodiments, v³ is 3 or 4. In some embodiments, v³ is 3, 4, or 5. In some embodiments, v³ is 4 or 5. In some embodiments, v³ is selected from the values represented in the compounds in Table 1.

As defined generally above, v⁴ is 0, 1, 2, 3, 4, or 5. In some embodiments, v⁴ is 0. In some embodiments, v⁴ is 1. In some embodiments, v⁴ is 2. In some embodiments, v⁴ is 3. In some embodiments, v⁴ is 4. In some embodiments, v⁴ is 5. In some embodiments, v⁴ is 0 or 1. In some embodiments, v⁴ is 0, 1, or 2. In some embodiments, v⁴ is 0, 1, 2, or 3. In some embodiments, v⁴ is 0, 1, 2, 3, or 4. In some embodiments, v⁴ is 1 or 2. In some embodiments, v⁴ is 1, 2, or 3. In some embodiments, v⁴ is 1, 2, 3, or 4. In some embodiments, v⁴ is 1, 2, 3, 4, or 5. In some embodiments, v⁴ is 2 or 3. In some embodiments, v⁴ is 2, 3, or 4. In some embodiments, v⁴ is 2, 3, 4, or 5. In some embodiments, v⁴ is 3 or 4. In some embodiments, v⁴ is 3, 4, or 5. In some embodiments, v⁴ is 4 or 5. In some embodiments, v⁴ is selected from the values represented in the compounds in Table 1.

As defined generally above, v⁵ is 0, 1, 2, 3, 4, or 5. In some embodiments, v⁵ is 0. In some embodiments, v⁵ is 1. In some embodiments, v⁵ is 2. In some embodiments, v⁵ is 3. In some embodiments, v⁵ is 4. In some embodiments, v⁵ is 5. In some embodiments, v⁵ is 0 or 1. In some embodiments, v⁵ is 0, 1, or 2. In some embodiments, v⁵ is 0, 1, 2, or 3. In some embodiments, v⁵ is 0, 1, 2, 3, or 4. In some embodiments, v⁵ is 1 or 2. In some embodiments, v⁵ is 1, 2, or 3. In some embodiments, v⁵ is 1, 2, 3, or 4. In some embodiments, v⁵ is 1, 2, 3, 4, or 5. In some embodiments, v⁵ is 2 or 3. In some embodiments, v⁵ is 2, 3, or 4. In some embodiments, v⁵ is 2, 3, 4, or 5. In some embodiments, v⁵ is 3 or 4. In some embodiments, v⁵ is 3, 4, or 5. In some embodiments, v⁵ is 4 or 5. In some embodiments, v⁵ is selected from the values represented in the compounds in Table 1.

As defined generally above, v⁶ is 0, 1, 2, 3, 4, or 5. In some embodiments, v⁶ is 0. In some embodiments, v⁶ is 1. In some embodiments, v⁶ is 2. In some embodiments, v⁶ is 3. In some embodiments, v⁶ is 4. In some embodiments, v⁶ is 5. In some embodiments, v⁶ is 0 or 1. In some embodiments, v⁶ is 0, 1, or 2. In some embodiments, v⁶ is 0, 1, 2, or 3. In some embodiments, v⁶ is 0, 1, 2, 3, or 4. In some embodiments, v⁶ is 1 or 2. In some embodiments, v⁶ is 1, 2, or 3. In some embodiments, v⁶ is 1, 2, 3, or 4. In some embodiments, v⁶ is 1, 2, 3, 4, or 5. In some embodiments, v⁶ is 2 or 3. In some embodiments, v⁶ is 2, 3, or 4. In some embodiments, v⁶ is 2, 3, 4, or 5. In some embodiments, v⁶ is 3 or 4. In some embodiments, v⁶ is 3, 4, or 5. In some embodiments, v⁶ is 4 or 5. In some embodiments, v⁶ is selected from the values represented in the compounds in Table 1.

As defined generally above, v⁷ is 0, 1, 2, 3, 4, or 5. In some embodiments, v⁷ is 0. In some embodiments, v⁷ is 1. In some embodiments, v⁷ is 2. In some embodiments, v⁷ is 3. In some embodiments, v⁷ is 4. In some embodiments, v⁷ is 5. In some embodiments, v⁷ is 0 or 1. In some embodiments, v⁷ is 0, 1, or 2. In some embodiments, v⁷ is 0, 1, 2, or 3. In some embodiments, v⁷ is 0, 1, 2, 3, or 4. In some embodiments, v⁷ is 1 or 2. In some embodiments, v⁷ is 1, 2, or 3. In some embodiments, v⁷ is 1, 2, 3, or 4. In some embodiments, v⁷ is 1, 2, 3, 4, or 5. In some embodiments, v⁷ is 2 or 3. In some embodiments, v⁷ is 2, 3, or 4. In some embodiments, v⁷ is 2, 3, 4, or 5. In some embodiments, v⁷ is 3 or 4. In some embodiments, v⁷ is 3, 4, or 5. In some embodiments, v⁷ is 4 or 5. In some embodiments, v⁷ is selected from the values represented in the compounds in Table 1.

As defined generally above, v⁸ is 0, 1, 2, 3, 4, or 5. In some embodiments, v⁸ is 0. In some embodiments, v⁸ is 1. In some embodiments, v⁸ is 2. In some embodiments, v⁸ is 3. In some embodiments, v⁸ is 4. In some embodiments, v⁸ is 5. In some embodiments, v⁸ is 0 or 1. In some embodiments, v⁸ is 0, 1, or 2. In some embodiments, v⁸ is 0, 1, 2, or 3. In some embodiments, v⁸ is 0, 1, 2, 3, or 4. In some embodiments, v⁸ is 1 or 2. In some embodiments, v⁸ is 1, 2, or 3. In some embodiments, v⁸ is 1, 2, 3, or 4. In some embodiments, v⁸ is 1, 2, 3, 4, or 5. In some embodiments, v⁸ is 2 or 3. In some embodiments, v⁸ is 2, 3, or 4. In some embodiments, v⁸ is 2, 3, 4, or 5. In some embodiments, v⁸ is 3 or 4. In some embodiments, v⁸ is 3, 4, or 5. In some embodiments, v⁸ is 4 or 5. In some embodiments, v⁸ is selected from the values represented in the compounds in Table 1.

In some embodiments, the present disclosure provides a compound of formula I-c wherein E¹ is —C(O)—, thereby forming a compound of formula I-c1:

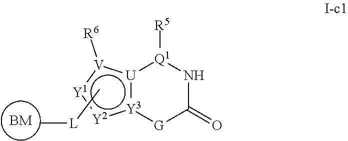

or a pharmaceutically acceptable salt thereof, wherein each of BM, L, R⁵, R⁶, Q¹, G, U, V, Y¹, Y², and Y³ is as defined in embodiments and classes and subclasses herein.

In some embodiments, the present disclosure provides a compound of formula I-c1 wherein U and V are C, thereby forming a compound of formula I-c2:

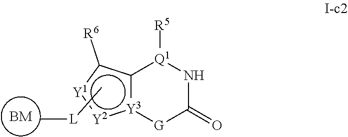

or a pharmaceutically acceptable salt thereof, wherein each of BM, L, R⁵, R⁶, Q¹, G, Y¹, Y², and Y³ is as defined in embodiments and classes and subclasses herein.

In some embodiments, the present disclosure provides a compound of formula I-c2 wherein Q¹ is CH, thereby forming a compound of formula I-c3:

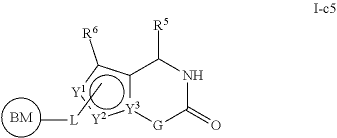

or a pharmaceutically acceptable salt thereof, wherein each of BM, L, R⁵, R⁶, G, Y¹, Y², and Y³ is as defined in embodiments and classes and subclasses herein.

In some embodiments, the present disclosure provides a compound of formula I-c3 wherein G is CH₂, thereby forming a compound of formula I-c4:

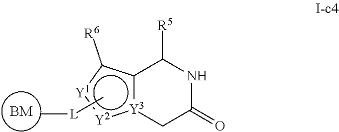

or a pharmaceutically acceptable salt thereof, wherein each of BM, L, R⁵, R⁶, Y¹, Y², and Y³ is as defined in embodiments and classes and subclasses herein.

In some embodiments, the present disclosure provides a compound of formula I-c3 wherein Y³ is N, thereby forming a compound of formula I-c5:

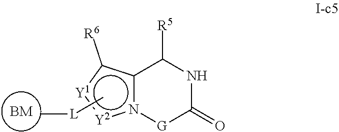

or a pharmaceutically acceptable salt thereof, wherein each of BM, L, R⁵, R⁶, G, Y¹, and Y² is as defined in embodiments and classes and subclasses herein.

In some embodiments, the present disclosure provides a compound of formula I-c3, I-c4, or I-c5, or a pharmaceutically acceptable salt thereof, wherein each of BM, L, R⁵, R⁶, G, Y¹, Y², and Y³ is as defined in embodiments and classes and subclasses herein.

In some embodiments, the present disclosure provides a compound of formula I-c2 wherein Y³ is N, and Y¹ or Y² is CH, thereby forming a compound of formula I-c6 or I-c7:

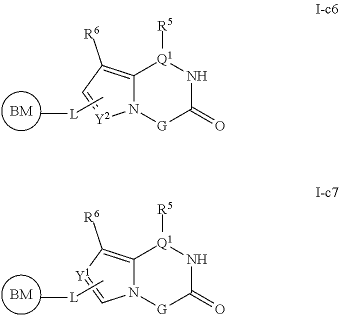

or a pharmaceutically acceptable salt thereof, wherein each of BM, L, R⁵, R⁶, Q¹, G, Y¹, and Y² is as defined in embodiments and classes and subclasses herein.

In some embodiments, the present disclosure provides a compound of formula I-c wherein G is a covalent bond, thereby forming a compound of formula I-c8, I-c9, or I-c10:

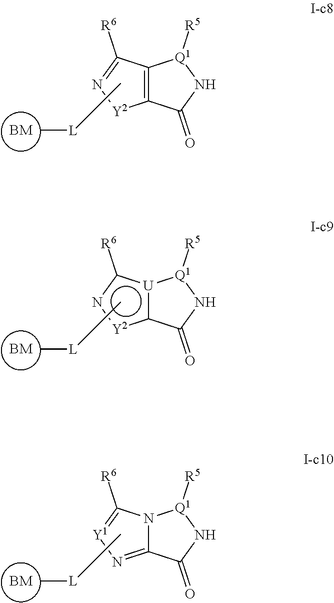

or a pharmaceutically acceptable salt thereof, wherein each of BM, L, R⁵, R⁶, Q¹, U, Y¹, and Y² is as defined in embodiments and classes and subclasses herein.

In some embodiments, the present disclosure provides a compound of formula I-c8, I-c9, or I-c10 wherein Q¹ is CH, thereby forming a compound of formula I-c11, I-c12, or I-c13:

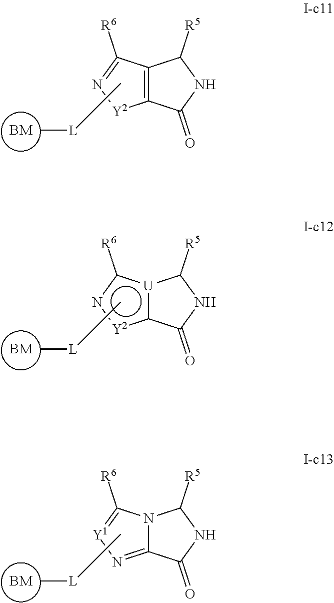

or a pharmaceutically acceptable salt thereof, wherein each of BM, L, R⁵, R⁶, U, Y¹, and Y² is as defined in embodiments and classes and subclasses herein.

In some embodiments, the present disclosure provides a compound of formula I-c3, I-c4, or I-c5 having the depicted stereochemistry at Q¹ when Q¹ is CH, thereby forming a compound of formula I-cc1, I-cc2, I-cc3, I-cc4, I-cc5, or I-cc6:

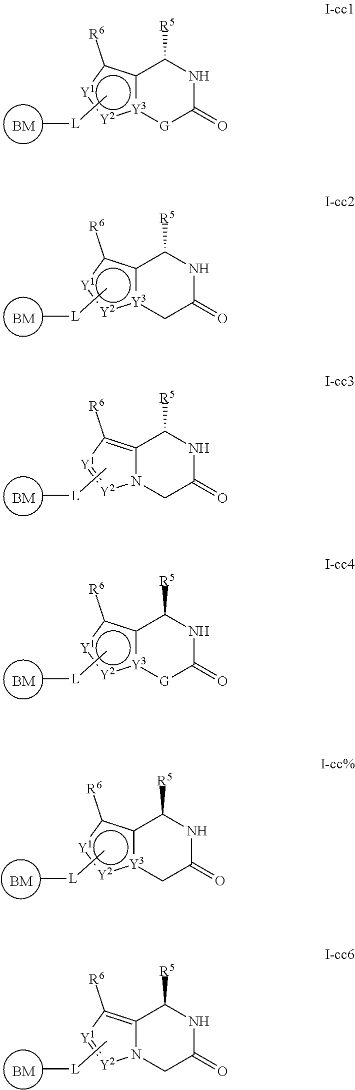

or a pharmaceutically acceptable salt thereof, wherein each of BM, L, R⁵, R⁶, G, Y¹, Y², and Y³ is as defined in embodiments and classes and subclasses herein.

In some embodiments, the present disclosure provides a compound of formula I-cc1 or I-cc2, or a pharmaceutically acceptable salt thereof, wherein each of BM, L, R⁵, R⁶, Y¹, Y², and Y³ is as defined in embodiments and classes and subclasses herein.

In some embodiments, the present disclosure provides a compound of formula I-c having the depicted point of attachment to -L-BM, thereby forming a compound of formula I-ccc1, I-ccc2, I-ccc3, I-ccc4, I-ccc5, or I-ccc6:

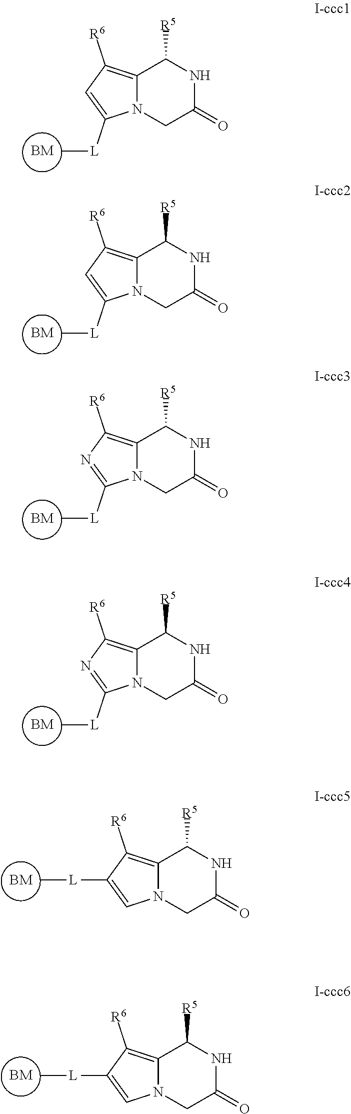

or a pharmaceutically acceptable salt thereof, wherein each of BM, L, R⁵, and R⁶ is as defined in embodiments and classes and subclasses herein.

In some embodiments, the present disclosure provides a compound of formula I-ccc1, I-ccc3, or I-ccc5, or a pharmaceutically acceptable salt thereof, wherein each of BM, L, R⁵, and R⁶ is as defined in embodiments and classes and subclasses herein.

In certain embodiments, the present disclosure provides a compound of formula I, in which PIK is a PI3K binding moiety of formula I-d0, thereby forming a compound of formula I-d:

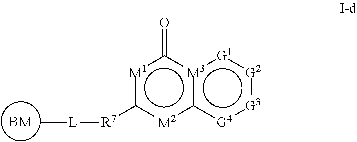

or a pharmaceutically acceptable salt thereof, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-BM;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3}), —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z¹ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

As defined generally above, G¹ is CH, N, or C—R^{G1}. In some embodiments, G¹ is CH or N. In some embodiments, G¹ is CH or C—R^{G1}. In some embodiments, G¹ is N or C—R^{G1}. In some embodiments G¹ is CH. In some embodiments, G¹ is N. In some embodiments, G¹ is C—R^{G1}. In some embodiments, G¹ is selected from the groups depicted in the compounds in Table 1.

As defined generally above, G² is CH, N, or C—R^{G2}. In some embodiments, G² is CH or N. In some embodiments, G² is CH or C—R^{G2}. In some embodiments, G² is N or C—R^{G2}. In some embodiments G² is CH. In some embodiments, G² is N. In some embodiments, G² is C—R^{G2}. In some embodiments, G² is selected from the groups depicted in the compounds in Table 1.

As defined generally above, one of G³ or G⁴ is C—R⁸ and the other is CH, N, or C—R^{G3}. In some embodiments, G³ is CR⁸ and G⁴ is CH, N, or C—R^{G3}. In some embodiments, G³ is CR⁸ and G⁴ is CH. In some embodiments, G³ is CR⁸ and G⁴ is N. In some embodiments, G³ is CR⁸ and G⁴ is C—R^{G3}. In some embodiments, G⁴ is CR⁸ and G³ is CH, N, or C—R^{G3}. In some embodiments, G⁴ is CR⁸ and G³ is CH. In some embodiments, G⁴ is CR⁸ and G³ is N. In some embodiments, G⁴ is CR⁸ and G³ is C—R^{G3}. In some embodiments, G² and G⁴ are selected from the groups depicted in the compounds in Table 1.

As defined generally above, M¹ is CH, C(R^{M1}), NH, or N(R^{M1}). In some embodiments, M¹ is CH. In some embodiments, M¹ is C(R^{M1}). In some embodiments, M¹ is NH. In some embodiments, M¹ is N(R^{M1}). In some embodiments, M¹ is CH or C(R^{M1}). In some embodiments, M¹ is CH or N(R^{M1}). In some embodiments, M¹ is C(R^{M1}) or N(R^{M1}) In some embodiments, M¹ is selected from the groups depicted in the compounds in Table 1.

As defined generally above, M² is O, CH, C(R^{M2}), N, NH, or N(R^{M2}). In some embodiments, M² is O. In some embodiments, M² is CH. In some embodiments, M² is C(R^{M2}). In some embodiments, M² is N. In some embodiments, M² is NH. In some embodiments, M² is N(R^{M2}). In some embodiments, M² is CH or C(R^{M2}). In some embodiments, M² is CH or N. In some embodiments, M² is C(R^{M2}) or N. In some embodiments, M² is C(R^{M2}) or N(R^{M2}). In some embodiments, M² is N or N(R^{M2}). In some embodiments, M² is selected from the groups depicted in the compounds in Table 1.

As defined generally above, M³ is C or N. In some embodiments, M³ is C. In some embodiments, M³ is N. In some embodiments, M³ is selected from the groups depicted in the compounds in Table 1.

As defined generally above, R⁷ is -L⁷-R^{7A} substituted with -L-BM. In some embodiments, R⁷ is —R^{7A} substituted with -L-BM.

In some embodiments, R⁷ (i.e. -L⁷-R^{7A} taken together) is

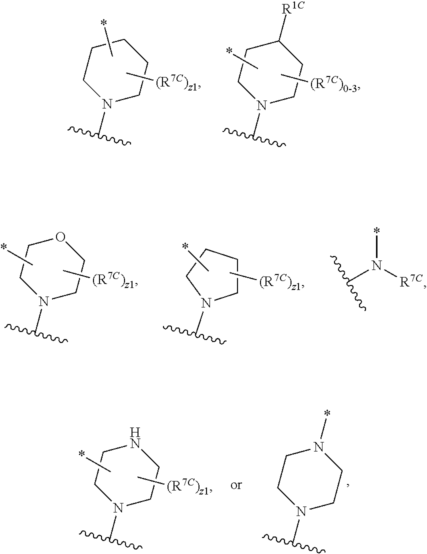

wherein [symbol] represents a covalent bond to L, and wherein R^{7C} and z¹ are as defined in the embodiments and classes and subclasses herein.

In some embodiments, R⁷ (i.e. -L⁷-R^{7A} taken together) is

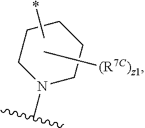

wherein [symbol] represents a covalent bond to L, and wherein R^{7C} and z¹ are as defined in the embodiments and classes and subclasses herein. In some embodiments, R⁷ (i.e. -L⁷-R^{7A} taken together) is

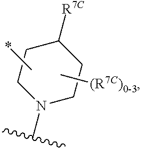

wherein R^{7C} is as defined in the embodiments and classes and subclasses herein. In some embodiments, R⁷ (i.e. -L⁷-R^{7A} taken together) is

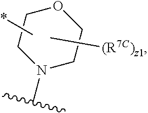

wherein R^{7C} is as defined in the embodiments and classes and subclasses herein. In some embodiments, R⁷ (i.e. -L⁷-R^{7A} taken together) is

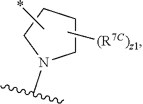

wherein R^{7C} is as defined in the embodiments and classes and subclasses herein. In some embodiments, R⁷ (i.e. -L⁷-R^{7A} taken together) is

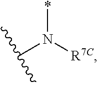

wherein R^{7C} is as defined in the embodiments and classes and subclasses herein. In some embodiments, R⁷ (i.e. -L⁷-R^{7A} taken together) is

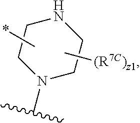

wherein R^{7C} is as defined in the embodiments and classes and subclasses herein. In some embodiments, R⁷ (i.e. -L⁷-R^{7A} taken together) is

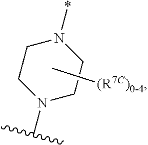

wherein R^{7C} is as defined in the embodiments and classes and subclasses herein. In some embodiments, R⁷ (i.e. -L⁷-R^{7A} taken together) is

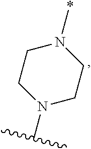

wherein R^{7C} is as defined in the embodiments and classes and subclasses herein.

In some embodiments, R⁷ (i.e. -L⁷-R^{7A} taken together) is

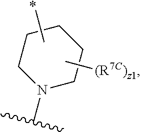

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R⁷ (i.e. -L⁷-R^{7A} taken together) is

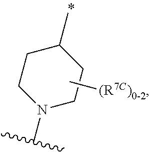

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R⁷ (i.e. -L⁷-R^{7A} taken together) is

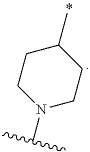 [SMILES: *N1CCC(C)CC1 ; "."]

In some embodiments, R⁷ (i.e. -L⁷-R^{7A} taken together) is

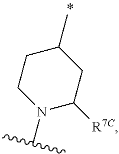

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R⁷ (i.e. -L⁷-R^{7A} taken together) is

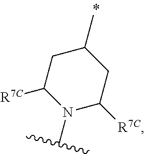

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein.

In some embodiments, R⁷ (i.e. -L⁷-R^{7A} taken together) is

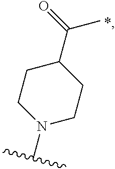

wherein R is as defined in the embodiments and classes and subclasses herein. In some embodiments, R⁷ (i.e. -L⁷-R^{7A} taken together) is

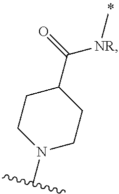

wherein R is as defined in the embodiments and classes and subclasses herein. In some embodiments, R⁷ (i.e. -L⁷-R^{7A} taken together) (i.e. -L⁷-R^{7A} taken together) is

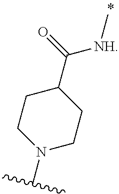 [SMILES: *N1CCC(C(=O)NC)CC1]

In some embodiments, R⁷ (i.e. -L⁷-R^{7A} taken together) is

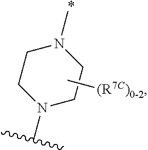

wherein each instance of R^{7C} is independently —C(O)R, —C(O)OR, —C(O)NR₂, or an optionally substituted C₁₋₆ aliphatic, wherein R is as defined in the embodiments and classes and subclasses herein. In some embodiments, R⁷ (i.e. -L⁷-R^{7A} taken together) is

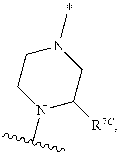

wherein each instance of R^{7C} is independently —C(O)R, —C(O)OR, —C(O)NR₂, or an optionally substituted C₁₋₆ aliphatic, wherein R is as defined in the embodiments and classes and subclasses herein. In some embodiments, R⁷ (i.e. -L⁷-R^{7A} taken together) is

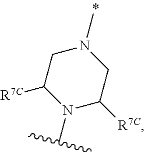

wherein each instance of R^{7C} is independently —C(O)R, —C(O)OR, —C(O)NR₂, or an optionally, substituted C₁₋₆ aliphatic, wherein R is as defined in the embodiments and classes and subclasses herein.

In some embodiments, R⁷ (i.e. -L⁷-R^{7A} taken together) is

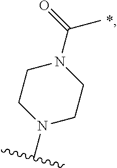

wherein R is as defined in the embodiments and classes and subclasses herein. In some embodiments, R⁷ (i.e. -L⁷-R^{7A} taken together) is

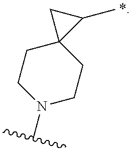 [SMILES: *N1CCC2(CC1)CC2C]

In some embodiments, R⁷ (i.e. -L⁷-R^{7A} taken together) is

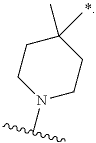 [SMILES: *N1CCC(C)(C)CC1]

In some embodiments, R⁷ (i.e. -L⁷-R^{7A} taken together) is

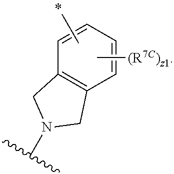

In some embodiments, R⁷ (i.e. -L⁷-R^{7A} taken together) is

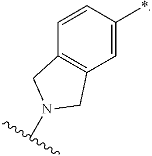

In some embodiments, R⁷ (i.e. -L⁷-R^{7A} taken together) is

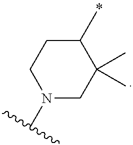 [SMILES: *N1CCC(C)C(C)(C)C1 ; "."]

In some embodiments, R⁷ (i.e. -L⁷-R^{7A} taken together) is

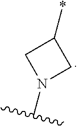 [SMILES: *C1CN(*)C1]

In some embodiments, R⁷ (i.e. -L⁷-R^{7A} taken together) is

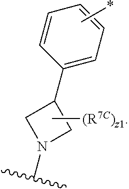

In some embodiments, R⁷ (i.e. -L⁷-R^{7A} taken together) is

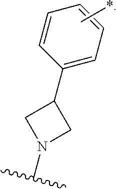

In some embodiments, R⁷ (i.e. -L⁷-R^{7A} taken together) is

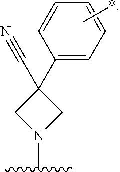

In some embodiments, R⁷ (i.e. -L⁷-R^{7A} taken together) is

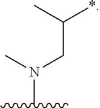

In some embodiments, R⁷ (i.e. -L⁷-R^{7A} taken together) is

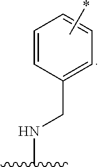

In some embodiments, R⁷ (i.e. -L⁷R^{7A} taken together) is

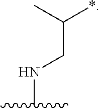

In some embodiments, R⁷ (i.e. -L⁷-R^{7A} taken together) is

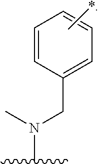

In some embodiments, R⁷ (i.e. -L⁷-R^{7A} taken together) is

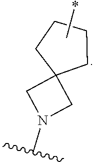

In some embodiments, R⁷ (i.e. -L⁷-R^{7A} taken together) is

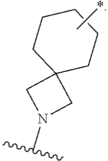

In some embodiments, R⁷ (i.e. -L⁷-R^{7A} taken together) is

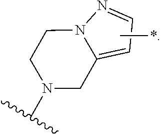

. In some embodiments, R⁷ (i.e. -L⁷-R^{7A} taken together) is

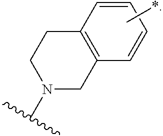

In some embodiments, R⁷ (i.e. -L⁷-R^{7A} taken together) is

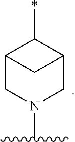 [SMILES: *C1C2CC1CN(*)C2]

In some embodiments, R⁷ (i.e. -L⁷-R^{7A} taken together) is

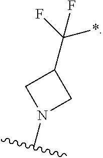

In some embodiments, R⁷ i.e. -L⁷-R^{7A} taken together) is

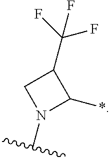

In some embodiments, R⁷ (i.e. -L⁷-R^{7A} taken together) is

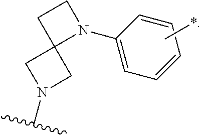

In some embodiments, R⁷ i.e. -L⁷-R^{7A} taken together) is

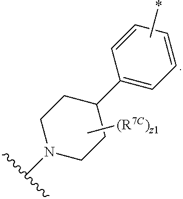

In some embodiments, R⁷ (i.e. -L⁷-R^{7A} taken together) is

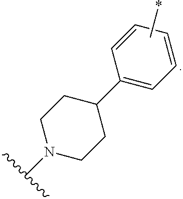

In some embodiments, R⁷ (i.e. -L⁷-R^{7A} taken together) is

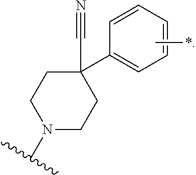

In some embodiments, R⁷ (i.e. -L⁷-R^{7A} taken together) is

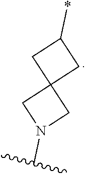 [SMILES: *C1CC2(C1)CN(*)C2]

In some embodiments, R⁷ (i.e. -L⁷-R^{7A} taken together) is

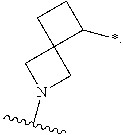

In some embodiments, R⁷ (i.e. -L⁷-R^{7A} taken together) is

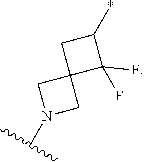 [SMILES: *C1CC2(CN(*)C2)C1(F)F]

In some embodiments, R⁷ (i.e. -L⁷-R^{7A} taken together) is

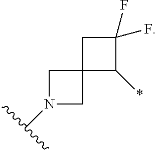 [SMILES: *C1C(F)(F)CC12CN(*)C2]

In some embodiments, R⁷ (i.e. -L⁷-R^{7A} taken together) is

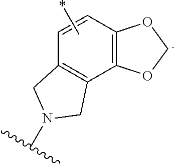

In some embodiments, R⁷ (i.e. -L⁷-R^{7A} taken together) is

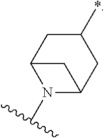

In some embodiments, R⁷ is selected from the groups depicted in the compounds in Table 1.

As defined generally above, R⁷ is -L⁸-R^{8A}. In some embodiments, R⁸ (i.e. -L⁸-R^{8A} taken together) is halogen. In some embodiments, R⁸ (i.e. -L⁸-R^{8A} taken together) is Br. In some embodiments, R⁸ (i.e. -L⁸-R^{8A} taken together) is —CH(R^{L3})N(H)—R^{8A}, wherein R and R^{8A} are as defined in the embodiments and classes and subclasses herein. In some embodiments, R⁸ (i.e. -L⁸-R^{8A} taken together) is —CH₂N(H)—R^{8A}, wherein R and R^{8A} are as defined in the embodiments and classes and subclasses herein. In some embodiments, R⁸ (i.e. -L⁸-R^{8A} taken together) is —CH(R^{L3})N(R)—R^{8A}, wherein R and R^{8A} are as defined in the embodiments and classes and subclasses herein. In some embodiments, R⁸ (i.e. -L⁸-R^{8A} taken together) is —CH(R^{L3})O—R^{8A}, wherein R and R^{8A} are as defined in the embodiments and classes and subclasses herein. In some embodiments, R⁸ (i.e. -L⁸-R^{8A} taken together) is —N(H)—R^{8A}, wherein R^{8A} is as defined in the embodiments and classes and subclasses herein. In some embodiments, R⁸ (i.e. -L⁸-R^{8A} taken together) is —CH₂—R^{8A}, wherein R^{8A} is as defined in the embodiments and classes and subclasses herein. In some embodiments, R⁸ (i.e. -L⁸-R^{8A} taken together) is —CH(R^{L3})—R^{8A}, wherein R^{8A} is as defined in the embodiments and classes and subclasses herein. In some embodiments, R⁸ (i.e. -L⁸-R^{8A} taken together) is —CH(R^{L3})N(H)—R^{8A}, wherein R is as defined in the embodiments and classes and subclasses herein, and wherein R^{8A} is R^{B} substituted by z² instances of R^{8C}. In some embodiments, R⁸ is —R^{8A}.

In some embodiments, R⁸ is —CH(CH₃)N(R)—R^{8A}, —CH(R^{L3})N(H)—R^{8A}, —CH(CH₃)N(H)—R^{8A}, or —R^{8A}. In some embodiments, R⁸ is —CH(R^{L3})N(H)—R^{8A}, —CH(R^{L3})N(R)—R^{8A}, —CH(R^{L3})O—R^{8A}, —N(H)—R^{8A}, —CH₂—R^{8A}, —CH(R^{L3})—R^{8A}, or —R^{8A}. In some embodiments, R⁸ is —CH(R^{L3})N(H)—R^{8A}, —CH(R^{L3})N(R)—R^{8A}, —N(H)—R^{8A}, or —R^{8A}. In some embodiments, R⁸ is —CH(R^{L3})N(H)—R^{8A}, —N(H)—R^{8A}, or —R^{8A}. In some embodiments, R⁸ is —CH(R^{L3})N(H)—R^{8A} or —R^{8A}. In some embodiments, R⁸ is —CH(CF₃)N(H)—R^{8A} or —R^{8A}.

In some embodiments, R⁸ is —CH(CH₃)N(H)—R^{8A}. In some embodiments, R⁸ is —CH(CH₃)O—R^{8A}. In some embodiments, R⁸ is —N(CH₃)—R^{8A}. In some embodiments, R⁸ is —CH(CH₃)—R^{8A}.

In some embodiments, R⁸ (i.e. -L⁸-R^{8A} taken together) is

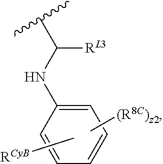

wherein R, R^{CyB}, R^{8C} and z² are as defined in the embodiments and classes and subclasses herein. In some embodiments, R⁸ (i.e. -L⁸-R^{8A} taken together) is

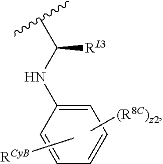

wherein R, R^{CyB}, R^{8C} and z² are as defined in the embodiments and classes and subclasses herein. In some embodiments, R⁸ (i.e. -L⁸-R^{8A} taken together) is

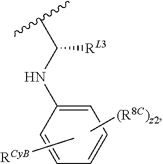

wherein R, R^{CyB}, R^{8C} and z² are as defined in the embodiments and classes and subclasses herein.

In some embodiments, R⁸ (i.e. -L⁸-R^{8A} taken together) is

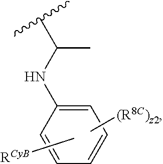

wherein R^{CyB}, R^{8C} and z² are as defined in the embodiments and classes and subclasses herein. In some embodiments, R⁸ (i.e. -L⁸-R^{8A} taken together) is

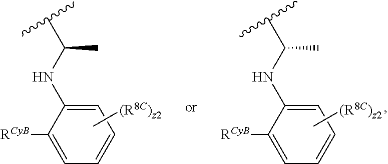

wherein R^{CyB}, R^{8C} and z² are as defined in the embodiments and classes and subclasses herein. In some embodiments, R⁸ (i.e. -L⁸-R^{8A} taken together) is

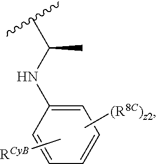

wherein R^{CyB}, R^{8C} and z² are as defined in the embodiments and classes and subclasses herein. In some embodiments, R⁸ (i.e. -L⁸-R^{8A} taken together) is

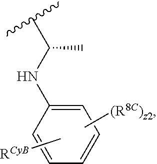

wherein R^{CyB}, R^{8C} and z² are as defined in the embodiments and classes and subclasses herein.

In some embodiments, R⁸ (i.e. -L⁸-R^{8A} taken together) is

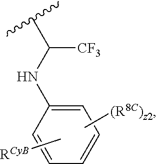

wherein R^{CyB}, R^{8C} and z² are as defined in the embodiments and classes and subclasses herein. In some embodiments, R⁸ (i.e. -L⁸-R^{8A} taken together) is

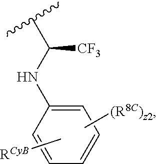

wherein R^{CyB}, R^{8C} and z² are as defined in the embodiments and classes and subclasses herein. In some embodiments, R⁸ (i.e. -L⁸-R^{8A} taken together) is

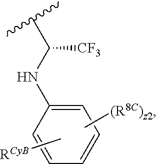

wherein R^{CyB}, R^{8C} and z² are as defined in the embodiments and classes and subclasses herein.

In some embodiments, R⁸ (i.e. -L⁸-R^{8A} taken together) is

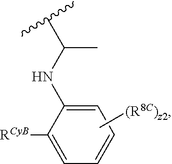

wherein R^{CyB} and R^{8C} is as defined in the embodiments and classes and subclasses herein. In some embodiments, R⁸ (i.e. -L⁸-R^{8A} taken together) is

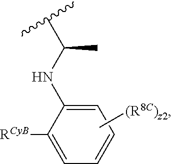

wherein R^{CyB} and R^{8C} is as defined in the embodiments and classes and subclasses herein. In some embodiments, R⁸ (i.e. -L⁸-R^{8A} taken together) is

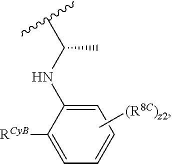

wherein R^{CyB} and R^{8C} is as defined in the embodiments and classes and subclasses herein.

In some embodiments, R⁸ (i.e. -L⁸-R^{8A} taken together) is

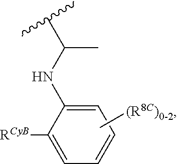

wherein R^{CyB} and R^{8C} is as defined in the embodiments and classes and subclasses herein. In some embodiments, R⁸ (i.e. -L⁸-R^{8A} taken together) is

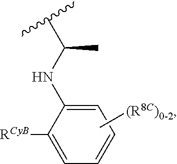

wherein R^{CyB} and R^{8C} is as defined in the embodiments and classes and subclasses herein. In some embodiments, R⁸ (i.e. -L⁸-R^{8A} taken together) is

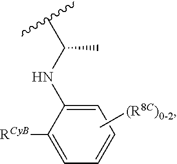

wherein R^{CyB} and R^{8C} is as defined in the embodiments and classes and subclasses herein.

In some embodiments, R⁸ (i.e. -L⁸-R^{8A} taken together) is

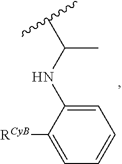

wherein R^{CyB} is as defined in the embodiments and classes and subclasses herein. In some embodiments, R⁸ (i.e. -L⁸-R^{8A} taken together) is

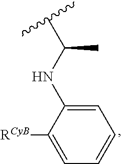

wherein R^{CyB} is as defined in the embodiments and classes and subclasses herein. In some embodiments, R⁸ (i.e. -L⁸-R^{8A} taken together) is

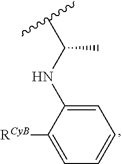

wherein R^{CyB} is as defined in the embodiments and classes and subclasses herein.

In some embodiments, R⁸ (i.e. -L⁸-R^{8A} taken together) is

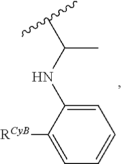

wherein R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

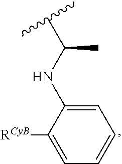

wherein R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

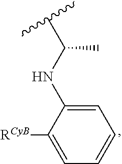

wherein R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

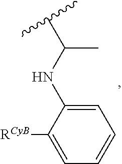

wherein R^{CyB} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

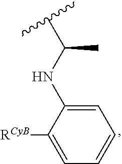

wherein R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

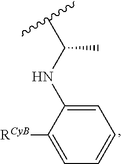

wherein R^{CyB} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

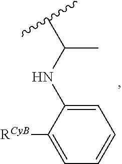

wherein R^{CyB} is —C(O)OR. In some embodiments, R⁸ (i.e. -L⁸-R^{8A} taken together) is

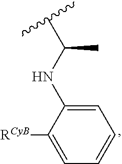

wherein R^{CyB} is —C(O)OR. In some embodiments, R⁸ (i.e. -L⁸-R^{8A} taken together) is

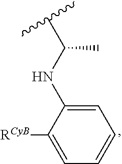

wherein R^{CyB} is —C(O)OR.

In some embodiments, R⁸ (i.e. -L⁸-R^{8A} taken together) is

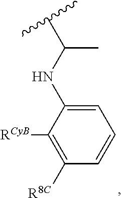

wherein R^{CyB} is as defined in the embodiments and classes and subclasses herein. In some embodiments, R⁸ (i.e. -L⁸-R^{8A} taken together) is

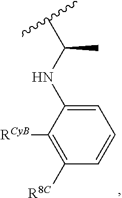

wherein R^{CyB} is as defined in the embodiments and classes and subclasses herein. In some embodiments, R⁸ (i.e. -L⁸-R^{8A} taken together) is

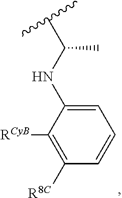

wherein R^{CyB} is as defined in the embodiments and classes and subclasses herein.

In some embodiments, R⁸ (i.e. -L⁸-R^{8A} taken together) is

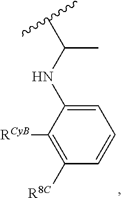

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

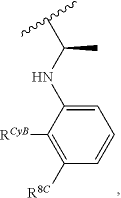

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

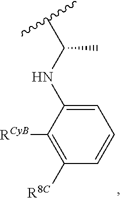

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen oxygen, and sulfur. In some embodiments, R⁸ (i.e. -L⁸-R^{8A} taken together) is

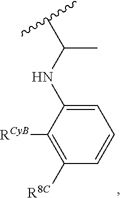

wherein R^{8C} is halogen, —CN, or an optionally substituted C₁₋₆ aliphatic, and R^{CyB} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

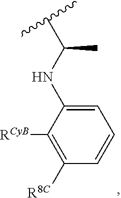

wherein R^{8C} is halogen, —CN, or an optionally substituted C₁₋₆ aliphatic, and R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

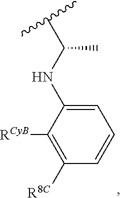

wherein R^{8C} is halogen, —CN, or an optionally substituted C₁₋₆ aliphatic, and R^{CyB} is —C(O)OR, —C(O)NR⁸, —S(O)R, —S(O)₂R, —S(O)NR⁸, —S(O)₂NR⁸.

In some embodiments, R⁸ (i.e. -L⁸-R^{8A} taken together) is

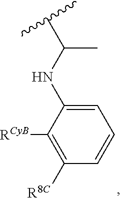

wherein R^{8C} is halogen, —CN, or an optionally substituted C₁₋₆ aliphatic, and R^{CyB} is —C(O)OR. In some embodiments, R⁸ (i.e. -L⁸-R^{8A} taken together) is

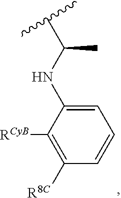

wherein R^{8C} is halogen, —CN, or an optionally substituted C₁₋₆ aliphatic, and R^{CyB} is —C(O)OR. In some embodiments, R⁸ (i.e. -L⁸-R^{8A} taken together) is

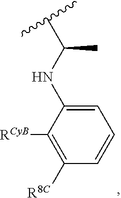

wherein R^{8C} is halogen, —CN, or an optionally substituted C₁₋₆ aliphatic, and R^{CyB} is —C(O)OR.

In some embodiments, R⁸ (i.e. -L⁸-R^{8A} taken together) is

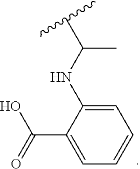 [SMILES: *C(C)Nc1ccccc1C(=O)O]

In some embodiments, R⁸ (i.e. -L⁸-R^{8A} taken together) is

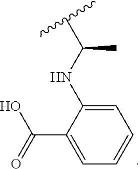 [SMILES: *[C@@H](C)Nc1ccccc1C(=O)O]

In some embodiments, R⁸ (i.e. -L⁸-R^{8A} taken together) is

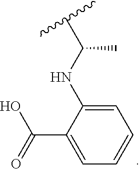 [SMILES: *[C@H](C)Nc1ccccc1C(=O)O]

In some embodiments, R⁸ (i.e. -L⁸-R^{8A} taken together) is

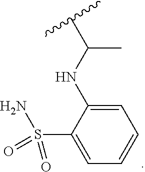 [SMILES: *C(C)Nc1ccccc1S(N)(=O)=O]

In some embodiments, R⁸ (i.e. -L⁸-R^{8A} taken together) is

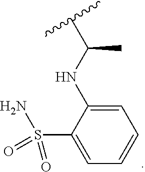 [SMILES: *[C@@H](C)Nc1ccccc1S(N)(=O)=O]

In some embodiments, R⁸ (i.e. -L⁸-R^{8A} taken together) is

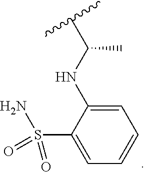 [SMILES: *[C@H](C)Nc1ccccc1S(N)(=O)=O]

In some embodiments, R⁸ (i.e. -L⁸-R^{8A} taken together) is

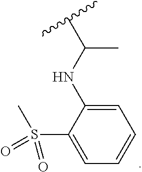 [SMILES: *C(C)Nc1ccccc1S(C)(=O)=O]

In some embodiments, R⁸ (i.e. -L⁸-R^{8A} taken together) is

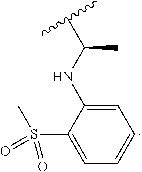 [SMILES: *[C@@H](C)Nc1ccccc1S(C)(=O)=O ; "."]

. In some embodiments, R⁸ (i.e. -L⁸-R^{8A} taken together) is

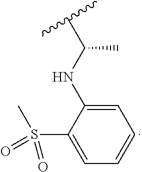 [SMILES: *[C@H](C)Nc1ccccc1S(C)(=O)=O ; "."]

In some embodiments, R⁸ (i.e. -L⁸-R^{8A} taken together) is

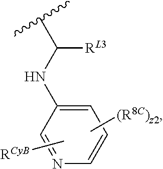

wherein R, R^{CyB}, R^{8C} and z² are as defined in the embodiments and classes and subclasses herein. In some embodiments, R⁸ (i.e. -L⁸-R^{8A} taken together) is

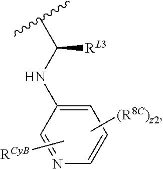

wherein R, R^{CyB}, R^{8C} and z² are as defined in the embodiments and classes and subclasses herein. In some embodiments, R⁸ (i.e. -L⁸-R^{8A} taken together) is

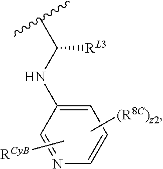

wherein R, R^{CyB}, R^{8C} and z² are as defined in the embodiments and classes and subclasses herein.

In some embodiments, R⁸ (i.e. -L⁸-R^{8A} taken together) is

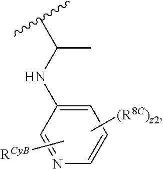

wherein R^{CyB}, R^{8C} and z² are as defined in the embodiments and classes and subclasses herein. In some embodiments, R⁸ (i.e. -L⁸-R^{8A} taken together) is

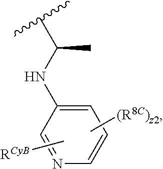

wherein R^{CyB}, R^{8C} and z² are as defined in the embodiments and classes and subclasses herein. In some embodiments, R⁸ (i.e. -L⁸-R^{8A} taken together) is

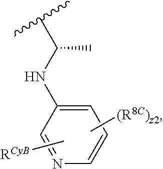

wherein R^{CyB}, R^{8C} and z² are as defined in the embodiments and classes and subclasses herein.

In some embodiments, R⁸ (i.e. -L⁸-R^{8A} taken together) is

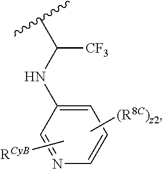

wherein R^{CyB}, R^{8C} and z² are as defined in the embodiments and classes and subclasses herein. In some embodiments, R⁸ (i.e. -L⁸-R^{8A} taken together) is

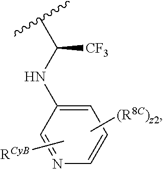

wherein R^{CyB}, R^{8C} and z² are as defined in the embodiments and classes and subclasses herein. In some embodiments, R⁸ (i.e. -L⁸-R^{8A} taken together) is

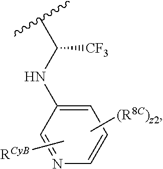

wherein R^{CyB}, R^{8C} and z² are as defined in the embodiments and classes and subclasses herein.

In some embodiments, R⁸ (i.e. -L⁸-R^{8A} taken together) is

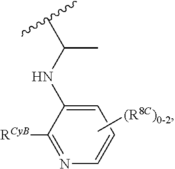

wherein R^{CyB} and R^{8C} is as defined in the embodiments and classes and subclasses herein. In some embodiments, R⁸ (i.e. -L⁸-R^{8A} taken together) is

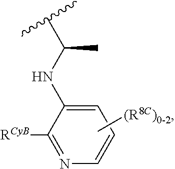

wherein R^{CyB} and R^{8C} is as defined in the embodiments and classes and subclasses herein. In some embodiments, R⁸ (i.e. -L⁸-R^{8A} taken together) is

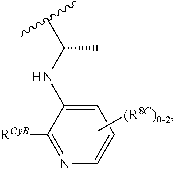

wherein R^{CyB} and R^{8C} is as defined in the embodiments and classes and subclasses herein.

In some embodiments, R⁸ (i.e. -L⁸-R^{8A} taken together) is

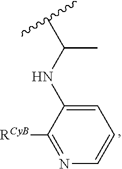

N wherein R^{CyB} is as defined in the embodiments and classes and subclasses herein. In some embodiments, R⁸ (i.e. -L⁸-R^{8A} taken together) is

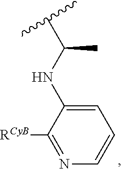

wherein R^{CyB} is as defined in the embodiments and classes and subclasses herein. In some embodiments, R⁸ (i.e. -L⁸-R^{8A} taken together) is

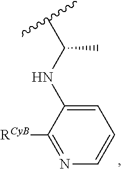

wherein R^{CyB} is as defined in the embodiments and classes and subclasses herein.

In some embodiments, R⁸ (i.e. -L⁸-R^{8A} taken together) is

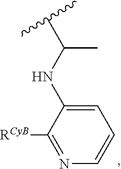

wherein R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

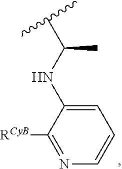

wherein R^{8C} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

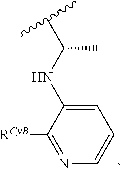

wherein R^{8C} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

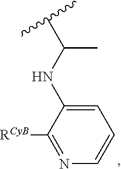

wherein R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

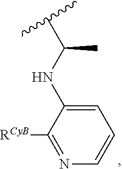

wherein R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

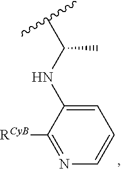

wherein R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

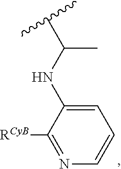

wherein R^{8C} is —C(O)OR. In some embodiments, R⁸ (i.e. -L⁸-R^{8A} taken together) is

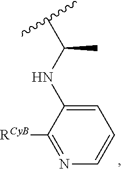

wherein R^{8C} is —C(O)OR. In some embodiments, R⁸ (i.e. -L⁸-R^{8A} taken together) is

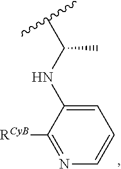

wherein R^{8C} is —C(O)OR.

In some embodiments, R⁸ (i.e. -L⁸-R^{8A} taken together) is

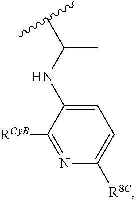

wherein R^{8C} and R^{CyB} are as defined in the embodiments and classes and subclasses herein. In some embodiments, R⁸ (i.e. -L⁸-R^{8A} taken together) is

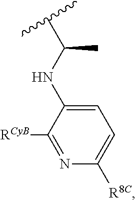

wherein R^{8C} and R^{CyB} are as defined in the embodiments and classes and subclasses herein. In some embodiments, R⁸ (i.e. -L⁸-R^{8A} taken together) is

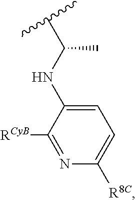

wherein R^{8C} and R^{CyB} are as defined in the embodiments and classes and subclasses herein.

In some embodiments, R⁸ (i.e. -L⁸-R^{8A} taken together) is

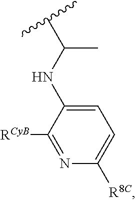

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

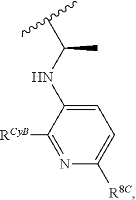

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur. In some embodiments, R⁸ (i.e. -L⁸-R^{8A} taken together) is

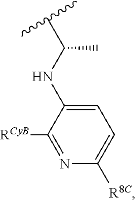

wherein R^{8C} is halogen, —CN, or an optionally substituted C₁₋₆ aliphatic, and R^{CyB} is halogen, —OH, —C(O)OR, —C(O)NR⁸, —S(O)R, —S(O)₂R, —S(O)NR⁸, —S(O)₂NR⁸, an optionally substituted C₁₋₆ aliphatic, or a 5-6 membered monocyclic heteroaryl ring having 1-4 heteroatoms independently selected from nitrogen, oxygen, and sulfur.

In some embodiments, R⁸ (i.e. -L⁸-R^{8A} taken together) is

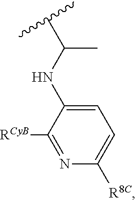

wherein R^{8C} is halogen, —CN, or an optionally substituted C₁₋₆ aliphatic, and R^{CyB} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

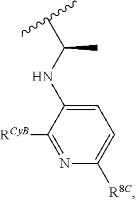

wherein R^{8C} is halogen, —CN, or an optionally substituted C₁₋₆ aliphatic, and R^{8C} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

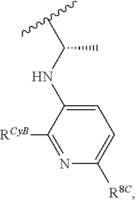

wherein R^{8C} is halogen, —CN, or an optionally substituted C₁₋₆ aliphatic, and R^{CyB} is —C(O)OR, —C(O)NR⁸, —S(O)R, —S(O)₂R, —S(O)NR⁸, —S(O)₂NR⁸. In some embodiments, R⁸ (i.e. -L⁸-R^{8A} taken together) is

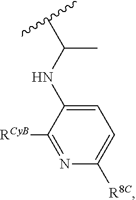

wherein R^{8C} is halogen, —CN, or an optionally substituted C₁₋₆ aliphatic, and R^{CyB} is —C(O)OR. In some embodiments, R⁸ (i.e. -L⁸-R^{8A} taken together) is

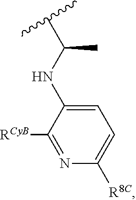

wherein R^{8C} is halogen, —CN, or an optionally substituted C₁₋₆ aliphatic, and R^{CyB} is —C(O)OR. In some embodiments, R⁸ (i.e. -L⁸-R^{8A} taken together) is

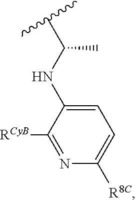

wherein R^{8C} is halogen, —CN, or an optionally substituted C₁₋₆ aliphatic, and R^{CyB} is —C(O)OR.

In some embodiments, R⁸ (i.e. -L⁸-R^{8A} taken together) is

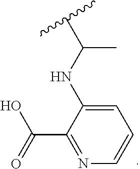 [SMILES: *C(C)Nc1cccnc1C(=O)O]

In some embodiments, R⁸ (i.e. -L⁸-R^{8A} taken together) is

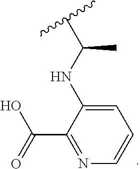 [SMILES: *[C@@H](C)Nc1cccnc1C(=O)O]

In some embodiments, R⁸ (i.e. -L⁸-R^{8A} taken together) is

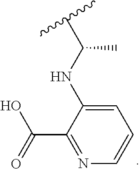 [SMILES: *[C@H](C)Nc1cccnc1C(=O)O]

In some embodiments, R⁸ (i.e. -L⁸-R^{8A} taken together) is

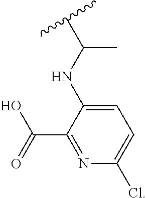 [SMILES: *C(C)Nc1ccc(Cl)nc1C(=O)O]

In some embodiments, R⁸ (i.e. -L⁸-R^{8A} taken together) is

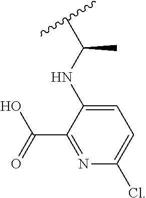 [SMILES: *[C@@H](C)Nc1ccc(Cl)nc1C(=O)O]

In some embodiments, R⁸ (i.e. -L⁸-R^{8A} taken together) is

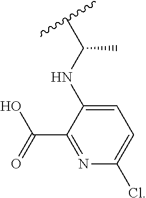 [SMILES: *[C@H](C)Nc1ccc(Cl)nc1C(=O)O]

In some embodiments, R⁸ (i.e. -L⁸-R^{8A} taken together) is

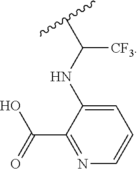 [SMILES: *C(Nc1cccnc1C(=O)O)C(F)(F)F]

In some embodiments, R⁸ (i.e. -L⁸-R^{8A} taken together) is

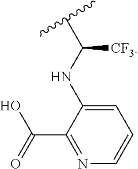 [SMILES: *[C@H](Nc1cccnc1C(=O)O)C(F)(F)F]

In some embodiments, R⁸ (i.e. -L⁸-R^{8A} taken together) is

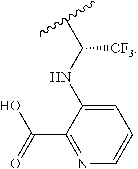 [SMILES: *[C@@H](Nc1cccnc1C(=O)O)C(F)(F)F]

In some embodiments, R⁸ (i.e. -L⁸-R^{8A} taken together) is

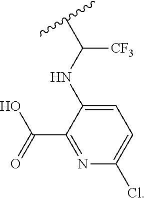 [SMILES: *C(Nc1ccc(Cl)nc1C(=O)O)C(F)(F)F]

In some embodiments, R⁸ (i.e. -L⁸-R^{8A} taken together) is

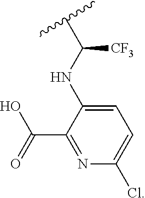 [SMILES: *[C@H](Nc1ccc(Cl)nc1C(=O)O)C(F)(F)F]

In some embodiments, R⁸ (i.e. -L⁸-R^{8A} taken together) is

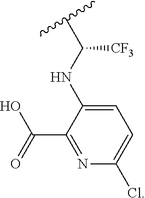 [SMILES: *[C@@H](Nc1ccc(Cl)nc1C(=O)O)C(F)(F)F]

In some embodiments, R⁸ is selected from the groups depicted in the compounds in Table 1.

As defined generally above, R^{G1} is -L^{G1}-R^{G1A}. In some embodiments, R^{G1} is —R^{G1A}.

In some embodiments, R^{G1} (i.e., -L^{G1}-R^{G1A} taken together) is halogen, —OH, C₁₋₆ alkoxy optionally substituted with 1-3 halogen, or C₁₋₆ aliphatic optionally substituted with 1-3 halogen. In some embodiments, R^{G1} is fluorine, chlorine, —OH, —OCH₃, —CH₃, —CHF₂, or —CF₃. In some embodiments, R^{G1} is fluorine. In some embodiments, R^{G1} is chlorine. In some embodiments, R^{G1} is —OH. In some embodiments, R^{G1} is —OCH₃. In some embodiments, R^{G1} is —CH₃. In some embodiments, R^{G1} is —CHF₂. In some embodiments, R^{G1} is —CF₃. In some embodiments, R^{G1} is selected from the groups depicted in the compounds in Table 1.

As defined generally above, R^{G2} is -L^{G2}-R^{G2A}. In some embodiments, R^{G2} is —R^{G2A}.

In some embodiments, R^{G2} (i.e., -L^{G2}-R^{G2A} taken together) is halogen, —OH, C₁₋₆ alkoxy optionally substituted with 1-3 halogen, or C₁₋₆ aliphatic optionally substituted with 1-3 halogen. In some embodiments, R^{G2} is fluorine, chlorine, —OH, —OCH₃, —CH₃, —CHF₂, or —CF₃. In some embodiments, R^{G2} is fluorine. In some embodiments, R^{G2} is chlorine. In some embodiments, R^{G2} is —OH. In some embodiments, R^{G2} is —OCH₃. In some embodiments, R^{G2} is methyl. In some embodiments, R^{G2} is —CHF₂. In some embodiments, R^{G2} is —CF₃. In some embodiments, R^{G2} is selected from the groups depicted in the compounds in Table 1.

As defined generally above, R^{G3} is -L^{G3}-R^{G3A}. In some embodiments, R^{G3} is —R^{G3A}.

In some embodiments, R^{G3} (i.e., -L^{G3}-R^{G3A} taken together) is halogen, —OH, C₁₋₆ alkoxy optionally substituted with 1-3 halogen, or C₁₋₆ aliphatic optionally substituted with 1-3 halogen. In some embodiments, R^{G3} is fluorine, chlorine, —OH, —OCH₃, —CH₃, —CHF₂, or —CF₃. In some embodiments, R^{G3} is fluorine. In some embodiments, R^{G3} is chlorine. In some embodiments, R^{G3} is —OH. In some embodiments, R^{G3} is —OCH₃. In some embodiments, R^{G3} is —CH₃. In some embodiments, R^{G3} is —CHF₂. In some embodiments, R^{G3} is —CF₃. In some embodiments, R^{G3} is selected from the groups depicted in the compounds in Table 1.

As defined generally above, R^{M1} is -L^{M1}-R^{M1A}. In some embodiments, R^{M1} is —R^{M1A}.

In some embodiments, R^{M1} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M1} is halogen, —CN, —OH, —O-(optionally substituted C₁₋₆ aliphatic), or an optionally substituted C₁₋₆ aliphatic. In some embodiments, R^{M1} is halogen, —OH, or C₁₋₃ aliphatic optionally substituted with 1-3 halogen. In some embodiments, R^{M1} is fluorine, chlorine, —OH, or —CH₃. In some embodiments, R^{G2} is methyl. In some embodiments, R^{M1} is deuterium. In some embodiments, R^{M1} is selected from the groups depicted in the compounds in Table 1.

As defined generally above, R^{M2} is -L^{M2}-R^{M2A}. In some embodiments, R^{M2} is —R^{M2A}.

In some embodiments, R^{M2} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M2} is halogen, —CN, —OH, —O-(optionally substituted C₁₋₆ aliphatic), or an optionally substituted C₁₋₆ aliphatic. In some embodiments, R^{M2} is halogen, —OH, or C₁₋₃ aliphatic optionally substituted with 1-3 halogen. In some embodiments, R^{M2} is fluorine, chlorine, —OH, or —CH₃. In some embodiments, R^{M2} is deuterium. In some embodiments, R^{M2} is selected from the groups depicted in the compounds in Table 1.

As defined generally above, L⁷ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁷ is a covalent bond. In some embodiments, L⁷ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁷ is a C₁₋₄ bivalent saturated or unsaturated, straight or branched hydrocarbon chain.

In some embodiments, L⁷ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁷ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L⁷ is a C₁₋₂ bivalent saturated or unsaturated hydrocarbon chain. In some embodiments, L⁷ is selected from the groups depicted in the compounds in Table 1.

As defined generally above, L⁸ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁸ is a covalent bond. In some embodiments, L⁸ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁸ is a C₁₋₄ bivalent saturated or unsaturated, straight or branched hydrocarbon chain.

In some embodiments, L⁸ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L⁸ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L⁸ is a C₁₋₂ bivalent saturated or unsaturated hydrocarbon chain.

In some embodiments, L⁸ is —N(R)C(O)— or —N(R)C(O)N(R)—. In some embodiments, L⁸ is —N(H)C(O)— or —N(H)C(O)N(H)—. In some embodiments, L⁸ is —N(R)C(O)—. In some embodiments, L⁸ is —N(H)C(O)—. In some embodiments, L⁸ is —N(R)C(O)N(R)—. In some embodiments, L⁸ is —N(H)C(O)N(H)—. In some embodiments, L⁸ is —N(R)—. In some embodiments, L⁸ is —N(H)—. In some embodiments, L⁸ is a covalent bond. In some embodiments, L⁸ is selected from the groups depicted in the compounds in Table 1.

As defined generally above, L^{G1} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G1} is a covalent bond. In some embodiments, L^{G1} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G1} is a C₁₋₄ bivalent saturated or unsaturated, straight or branched hydrocarbon chain.

In some embodiments, L^{G1} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G1} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{G1} is a C₁₋₂ bivalent saturated or unsaturated hydrocarbon chain. In some embodiments, L^{G1} is selected from the groups depicted in the compounds in Table 1.

As defined generally above, L^{G2} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G2} is a covalent bond. In some embodiments, L^{G2} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G2} is a C₁₋₄ bivalent saturated or unsaturated, straight or branched hydrocarbon chain. In some embodiments, L^{G2} is a covalent bond or —O—.

In some embodiments, L^{G2} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G2} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{G2} is a C₁₋₂ bivalent saturated or unsaturated hydrocarbon chain. In some embodiments, L^{G2} is selected from the groups depicted in the compounds in Table 1.

As defined generally above, L^{G3} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G3} is a covalent bond. In some embodiments, L^{G3} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G3} is a C₁₋₄ bivalent saturated or unsaturated, straight or branched hydrocarbon chain.

In some embodiments, L^{G3} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G3} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{G3} is a C₁₋₂ bivalent saturated or unsaturated hydrocarbon chain. In some embodiments, L^{G3} is selected from the groups depicted in the compounds in Table 1.

As defined generally above, L^{M1} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M1} is a covalent bond. In some embodiments, L^{M1} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M1} is a C₁₋₄ bivalent saturated or unsaturated, straight or branched hydrocarbon chain.

In some embodiments, L^{M1} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M1} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{M1} is a C₁₋₂ bivalent saturated or unsaturated hydrocarbon chain. In some embodiments, L^{M1} is selected from the groups depicted in the compounds in Table 1.

As defined generally above, L^{M2} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M2} is a covalent bond. In some embodiments, L^{M2} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M2} is a C₁₋₄ bivalent saturated or unsaturated, straight or branched hydrocarbon chain.

In some embodiments, L^{M2} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{M2} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{M2} is a C₁₋₂ bivalent saturated or unsaturated hydrocarbon chain.

In some embodiments, L^{M2} is —C(O)N(R)—, —C(O)N(R)CH₂—, or a covalent bond. In some embodiments, L^{M2} is —C(O)N(H)—, —C(O)N(H)CH₂—, or a covalent bond. In some embodiments, L^{M2} is —C(O)N(H)— or —C(O)N(H)CH₂—. In some embodiments, L^{M2} is —C(O)N(H)—. In some embodiments, L^{M2} is —C(O)N(H)CH₂—. In some embodiments, L^{M2} is selected from the groups depicted in the compounds in Table 1.

As defined generally above, R^{7A} is R^{J} or R^{K} substituted by z¹ instances of R^{7C}. In some embodiments, R^{7A} is R^{J}. In some embodiments, R^{7A} is R^{K} substituted by z¹ instances of R^{7C}.

In some embodiments, R^{7A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{7A} is substituted by z¹ instances of R^{7C}.

In some embodiments, R^{7A} is phenyl substituted by z¹ instances of R^{7C}. In some embodiments, R^{7A} is an 8-10 membered bicyclic heteroaryl ring having 1-4 heteroatoms independently selected from nitrogen, oxygen, and sulfur, wherein R^{7A} is substituted by z¹ instances of R^{7C}. In some embodiments, R^{7A} is phenyl or an 8-10 membered bicyclic heteroaryl ring having 1-4 heteroatoms independently selected from nitrogen, oxygen, and sulfur, wherein R^{7A} is substituted by z¹ instances of R^{7C}.

In some embodiments, R^{7A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{7A} is substituted by z¹ instances of R^{7C}.

In some embodiments, R^{7A} is phenyl substituted by z¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{7A} is an 8-10 membered bicyclic heteroaryl ring having 1-4 heteroatoms independently selected from nitrogen, oxygen, and sulfur, wherein R^{7A} is substituted by z¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{7A} is phenyl or an 8-10 membered bicyclic heteroaryl ring having 1-4 heteroatoms independently selected from nitrogen, oxygen, and sulfur, wherein R^{7A} is substituted by z¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{7A} is phenyl substituted by 1-3 instances of R^{7C}. In some embodiments, R^{7A} is phenyl substituted by 2 instances of R^{7C}. In some embodiments, R^{7A} is phenyl substituted by 1 instance of R^{7C}.

In some embodiments, R^{7A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{7A} is phenyl substituted by 1-3 instances of a group independently selected from halogen and C₁₋₃ aliphatic optionally substituted with 1-3 halogen. In some embodiments, R^{7A} is phenyl substituted by 1-3 instances of a group independently selected from fluorine, chlorine, —CH₃, —CHF₂, and —CF₃.

In some embodiments, R^{7A} is phenyl substituted by 2 instances of a group independently selected from halogen, —CN, —O-(optionally substituted C₁₋₆ aliphatic), and an optionally substituted C₁₋₆ aliphatic. In some embodiments, R^{7A} is phenyl substituted by 2 instances of a group independently selected from halogen and C₁₋₃ aliphatic optionally substituted with 1-3 halogen. In some embodiments, R^{7A} is phenyl substituted by 2 instances of a group independently selected from fluorine, chlorine, —CH₃, —CHF₂, and —CF₃.

In some embodiments, R^{7A} is phenyl substituted by one group selected from halogen, —CN, —O-(optionally substituted C₁₋₆ aliphatic), and an optionally substituted C₁₋₆ aliphatic. In some embodiments, R^{7A} is phenyl substituted by one halogen or C₁₋₃ aliphatic group optionally substituted with 1-3 halogen. In some embodiments, R^{7A} is phenyl substituted by one fluorine, chlorine, —CH₃, —CHF₂, or —CF₃.

In some embodiments, R^{7A} is

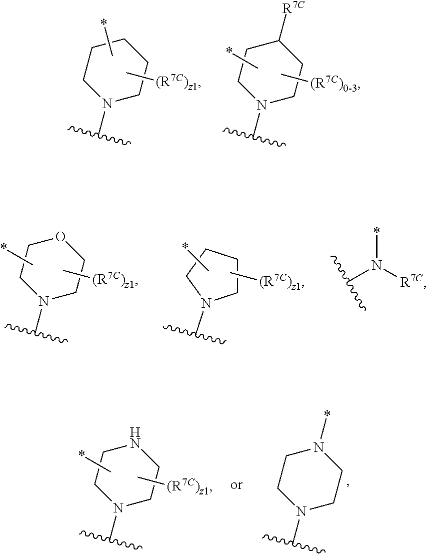

wherein [symbol] represents a covalent bond to L.

In some embodiments, R^{7A} is

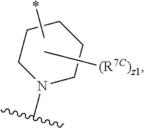

wherein [symbol] represents a covalent bond to L, and wherein R^{7C} and z¹ are as defined in the embodiments and classes and subclasses herein. In some embodiments, R^{7A} is

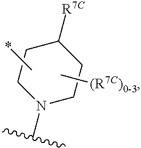

wherein R^{7C} is as defined in the embodiments and classes and subclasses herein. In some embodiments, R^{7A} is

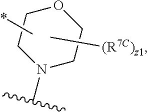

wherein R^{7C} is as defined in the embodiments and classes and subclasses herein. In some embodiments, R^{7A} is

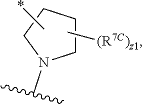

wherein R^{7C} is as defined in the embodiments and classes and subclasses herein. In some embodiments, R^{7A} is

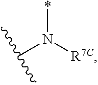

wherein R^{7C} is as defined in the embodiments and classes and subclasses herein. In some embodiments, R^{7A} is

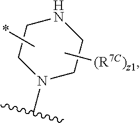

wherein R^{7C} is as defined in the embodiments and classes and subclasses herein. In some embodiments, R^{7A} is

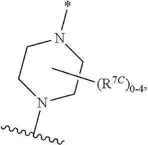

wherein R^{7C} is as defined in the embodiments and classes and subclasses herein. In some embodiments, R^{7A} is

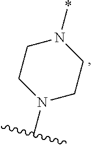

wherein R^{7C} is as defined in the embodiments and classes and subclasses herein.

In some embodiments, R^{7A} is

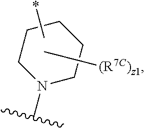

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R^{7A} is

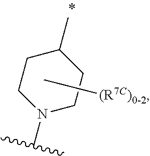

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R^{7A} is

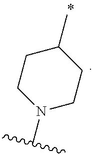 [SMILES: *N1CCC(C)CC1]

In some embodiments, R^{7A} is

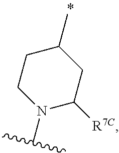

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein. In some embodiments, R^{7A} is

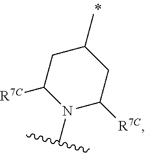

wherein each instance of R^{7C} is independently —S(O)₂R, —S(O)₂NR₂, —S(O)R, —S(O)NR₂, —S(O)(NR)R, —C(O)R, —C(O)OR, —C(O)NR₂, or an optionally substituted C₁₋₆ aliphatic, wherein R is as defined in the embodiments and classes and subclasses herein.

In some embodiments, R^{7A} is

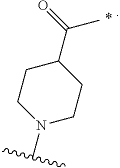 [SMILES: *N1CCC(C(C)=O)CC1]

In some embodiments, R^{7A} is

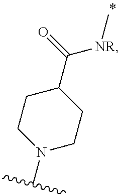

wherein R is as defined in the embodiments and classes and subclasses herein. In some embodiments, R^{7A} is

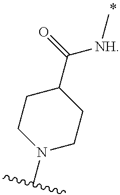 [SMILES: *N1CCC(C(=O)NC)CC1]

In some embodiments, R^{7A} is

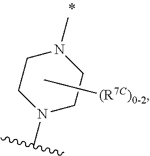

wherein each instance of R^{7C} is independently —C(O)R, —C(O)OR, —C(O)NR₂, or an optionally substituted C₁₋₆ aliphatic, wherein R is as defined in the embodiments and classes and subclasses herein. In some embodiments, R^{7A} is

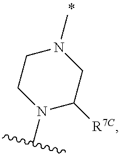

wherein each instance of R^{7C} is independently —C(O)R, —C(O)OR, —C(O)NR₂, or an optionally substituted C₁₋₆ aliphatic, wherein R is as defined in the embodiments and classes and subclasses herein. In some embodiments, R^{7A} is

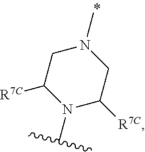

wherein each instance of R^{7C} is independently —C(O)R, —C(O)OR, —C(O)NR₂, or an optionally substituted C₁₋₆ aliphatic, wherein R is as defined in the embodiments and classes and subclasses herein.

In some embodiments, R^{7A} is

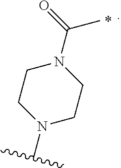 [SMILES: *N1CCN(C(C)=O)CC1]

In some embodiments, R^{7A} is

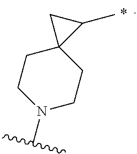 [SMILES: *N1CCC2(CC1)CC2C]

In some embodiments, R^{7A} is

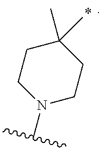 [SMILES: *N1CCC(C)(C)CC1]

In some embodiments, R^{7A} is

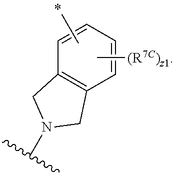

In some embodiments, R^{7A} is

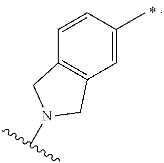 [SMILES: *N1Cc2ccc(C)cc2C1]

In some embodiments, R^{7A} is

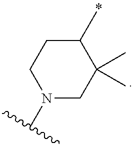 [SMILES: *N1CCC(C)C(C)(C)C1 ; "."]

In some embodiments, R^{7A} is

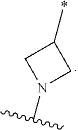

In some embodiments, R^{7A} is

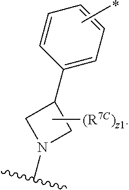

In some embodiments, R^{7A} is

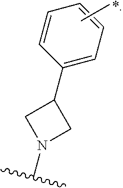

In some embodiments, R^{7A} is

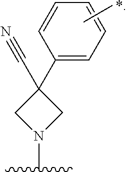

In some embodiments, R^{7A} is

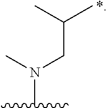

In some embodiments, R^{7A} is

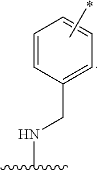

In some embodiments, R^{7A} is

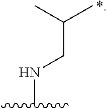

In some embodiments, R^{7A} is

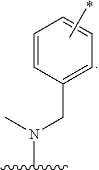

In some embodiments, R^{7A} is

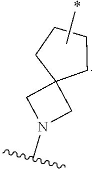

In some embodiments, R^{7A} is

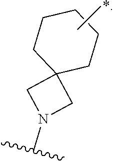

In some embodiments, R^{7A} is

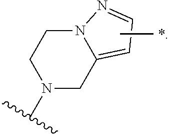

In some embodiments, R^{7A} is

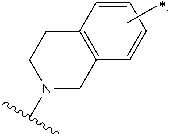

In some embodiments, R^{7A} is

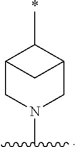

In some embodiments, R^{7A} is

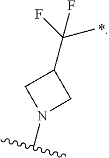

In some embodiments, R^{7A} is

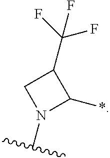

In some embodiments, R^{7A} is

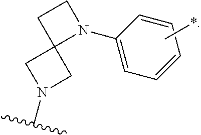

In some embodiments, R^{7A} is

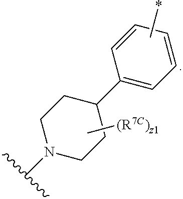

In some embodiments, R^{7A} is

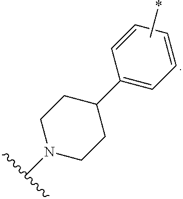

In some embodiments, R^{7A} is

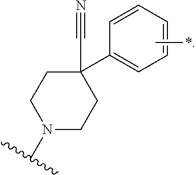

K In some embodiments, R^{7A} is

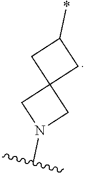 [SMILES: *C1CC2(C1)CN(*)C2]

In some embodiments, R^{7A} is

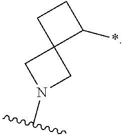

In some embodiments, R^{7A} is

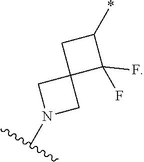 [SMILES: *C1CC2(CN(*)C2)C1(F)F]

In some embodiments, R^{7A} is

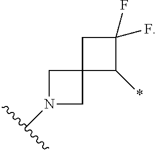 [SMILES: *C1C(F)(F)CC12CN(*)C2]

In some embodiments, R^{7A} is

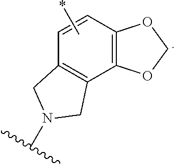

some embodiments, R^{7A} is

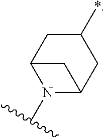

In some embodiments, R^{7A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{7A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{7A} is oxo. In some embodiments, R^{7A} is halogen. In some embodiments, R^{7A} is —CN. In some embodiments, R^{7A} is —NO₂. In some embodiments, R^{7A} is —OR. In some embodiments, R^{7A} is —SR. In some embodiments, R^{7A} is —NR₂. In some embodiments, R^{7A} is —S(O)₂R. In some embodiments, R^{7A} is —S(O)₂NR₂. In some embodiments, R^{7A} is —S(O)₂F. In some embodiments, R^{7A} is —S(O)R. In some embodiments, R^{7A} is —S(O)NR₂. In some embodiments, R^{7A} is —S(O)(NR)R. In some embodiments, R^{7A} is —C(O)R. In some embodiments, R^{7A} is —C(O)OR. In some embodiments, R^{7A} is —C(O)NR₂. In some embodiments, R^{7A} is —C(O)N(R)OR. In some embodiments, R^{7A} is —OC(O)R. In some embodiments, R^{7A} is —OC(O)NR₂. In some embodiments, R^{7A} is —N(R)C(O)OR. In some embodiments, R^{7A} is —N(R)C(O)R. In some embodiments, R^{7A} is —N(R)C(O)NR₂. In some embodiments, R^{7A} is —N(R)C(NR)NR₂. In some embodiments, R^{7A} is —N(R)S(O)₂NR₂. In some embodiments, R^{7A} is —N(R)S(O)₂R. In some embodiments, R^{7A} is —P(O)R₂. In some embodiments, R^{7A} is —P(O)(R)OR. In some embodiments, R^{7A} is —B(OR)₂. In some embodiments, R^{7A} is deuterium.

In some embodiments, R^{7A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{7A} is halogen, —CN, or —NO₂. In some embodiments, R^{7A} is —OR, —SR, or —NR₂. In some embodiments, R^{7A} is —S(O)₂R, —S(O)₂NR₂, —S(O)₂F, —S(O)R, —S(O)NR₂, or —S(O)(NR)R. In some embodiments, R^{7A} is —C(O)R, —C(O)OR, —C(O)NR₂, or —C(O)N(R)OR. In some embodiments, R^{7A} is —OC(O)R or —OC(O)NR₂. In some embodiments, R^{7A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{7A} is —P(O)R₂ or —P(O)(R)OR.

In some embodiments, R^{7A} is —OR, —OC(O)R, or —OC(O)NR₂. In some embodiments, R^{7A} is —SR, —S(O)₂R, —S(O)₂NR₂, —S(O)₂F, —S(O)R, —S(O)NR₂, or —S(O)(NR)R. In some embodiments, R^{7A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{7A} is —S(O)₂R, —S(O)₂NR₂, or —S(O)₂F. In some embodiments, R^{7A} is —S(O)R, —S(O)NR₂, or —S(O)(NR)R. In some embodiments, R^{7A} is —SR, —S(O)₂R, or —S(O)R. In some embodiments, R^{7A} is —S(O)₂NR₂, —S(O)NR₂, or —S(O)(NR)R. In some embodiments, R^{7A} is —S(O)₂NR₂ or —S(O)NR₂. In some embodiments, R^{7A} is —SR, —S(O)₂R, —S(O)₂NR₂, or —S(O)R.

In some embodiments, R^{7A} is —N(R)C(O)OR, —N(R)C(O)R, or —N(R)C(O)NR₂. In some embodiments, R^{7A} is —N(R)S(O)₂NR₂ or —N(R)S(O)₂R. In some embodiments, R^{7A} is —N(R)C(O)OR or —N(R)C(O)R. In some embodiments, R^{7A} is —N(R)C(O)NR₂ or —N(R)S(O)₂NR₂. In some embodiments, R^{7A} is —N(R)C(O)OR, —N(R)C(O)R, or —N(R)S(O)₂R.

In some embodiments, R^{7A} is —NR₂, —N(R)C(O)OR, —N(R)C(O)R, or —N(R)C(O)NR₂. In some embodiments, R^{7A} is —NR₂, —N(R)C(O)OR, or —N(R)C(O)R. In some embodiments, R^{7A} is —NR₂, —N(R)C(O)OR, —N(R)C(O)R, or —N(R)S(O)₂R.

In some embodiments, R^{7A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is a C₁₋₆ aliphatic chain substituted by z¹ instances of R^{7C}. In some embodiments, R^{7A} is phenyl substituted by z¹ instances of R^{7C}. In some embodiments, R^{7A} is naphthyl substituted by z¹ instances of R^{7C}. In some embodiments, R^{7A} is a 5-6 membered monocyclic heteroaryl ring having 1-4 heteroatoms independently selected from nitrogen, oxygen, and sulfur; wherein said ring is substituted by z¹ instances of R^{7C}. In some embodiments, R^{7A} is an 8-10 membered bicyclic heteroaryl ring having 1-4 heteroatoms independently selected from nitrogen, oxygen, and sulfur; wherein said ring is substituted by z¹ instances of R^{7C}. In some embodiments, R^{7A} is a 3-7 membered saturated or partially unsaturated monocyclic carbocyclic ring substituted by z¹ instances of R^{7C}. In some embodiments, R^{7A} is a 5-12 membered saturated or partially unsaturated bicyclic carbocyclic ring substituted by z¹ instances of R^{7C}. In some embodiments, R^{7A} is a 3-7 membered saturated or partially unsaturated monocyclic heterocyclic ring having 1-2 heteroatoms independently selected from nitrogen, oxygen, and sulfur; wherein said ring is substituted by z¹ instances of R^{7C}. In some embodiments, R^{7A} is a 7-12 membered saturated or partially unsaturated bicyclic heterocyclic ring having 1-4 heteroatoms independently selected from nitrogen, oxygen, and sulfur; wherein said ring is substituted by z¹ instances of R^{7C}.

In some embodiments, R^{7A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z¹ instances of R^{7C}. In some embodiments, R^{7A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z¹ instances of R^{7C}. In some embodiments, R^{7A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z¹ instances of R^{7C}. In some embodiments, R^{7A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is phenyl or naphthyl; each of which is substituted by z¹ instances of R^{7C}. In some embodiments, R^{7A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z¹ instances of R^{7C}. In some embodiments, R^{7A} is a 3-7 membered saturated or partially unsaturated monocyclic carbocyclic ring or a 5-12 membered saturated or partially unsaturated bicyclic carbocyclic ring; each of which is substituted by z¹ instances of R^{7C}. In some embodiments, R^{7A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is phenyl or a 5-6 membered monocyclic heteroaryl ring having 1-4 heteroatoms independently selected from nitrogen, oxygen, and sulfur; each of which is substituted by z¹ instances of R^{7C}. In some embodiments, R^{7A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z¹ instances of R^{7C}. In some embodiments, R^{7A} is naphthyl or an 8-10 membered bicyclic heteroaryl ring having 1-4 heteroatoms independently selected from nitrogen, oxygen, and sulfur; each of which is substituted by z¹ instances of R^{7C}. In some embodiments, R^{7A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is phenyl or a 3-7 membered saturated or partially unsaturated monocyclic carbocyclic ring; each of which is substituted by z¹ instances of R^{7C}. In some embodiments, R^{7A} is naphthyl or a 5-12 membered saturated or partially unsaturated bicyclic carbocyclic ring; each of which is substituted by z¹ instances of R^{7C}. In some embodiments, R^{7A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by z¹ instances of R^{7C}. In some embodiments, R^{7A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}.

In some embodiments, R^{7A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z¹ instances of R^{7C}. In some embodiments, R^{7A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z¹ instances of R^{7C}. In some embodiments, R^{7A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z¹ instances of R^{7C}.

In some embodiments, R^{7A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z¹ instances of R^{7C}. In some embodiments, R^{7A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z¹ instances of R^{7C}. In some embodiments, R^{7A} is a C₁₋₆ aliphatic chain, phenyl, or a 3-7 membered saturated or partially unsaturated monocyclic carbocyclic ring; each of which is substituted by z¹ instances of R^{7C}.

In some embodiments, R^{7A} is selected from the groups depicted in the compounds in Table 1.

As defined generally above, R^{8A} is R, R^{K} substituted by z² instances of R^{8C}, or Cy^{B}-R^{CyB} substituted by z² instances of R^{8C}. In some embodiments, R^{8A} is R^{J}. In some embodiments, R^{8A} is R^{K} substituted by z² instances of R^{8C}. In some embodiments, R^{8A} is Cy^{B}-R^{CyB} substituted by z² instances of R^{8C}.

In some embodiments, R^{8A} (i.e., -Cy^{B}-R^{CyB} taken together) is phenyl-R^{CyB}, naphthyl-R^{CyB}, cubanyl-R^{CyB}, adamantyl-R^{CyB}, a 5-6 membered monocyclic heteroaryl ring (substituted with —R^{CyB}) having 1-4 heteroatoms independently selected from nitrogen, oxygen, and sulfur, or an 8-10 membered bicyclic heteroaryl ring (substituted with —R^{CyB}) having 1-4 heteroatoms independently selected from nitrogen, oxygen, and sulfur; wherein R^{8A} is substituted by z² instances of R^{2C}.

In some embodiments, R^{8A} is phenyl-R^{CyB}; naphthyl-R^{CyB}; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{8A} is substituted by z² instances of R^{2C}. In some embodiments, R^{8A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{8A} is substituted by z² instances of R^{2C}. In some embodiments, R^{8A} is phenyl or an 8-10 membered bicyclic heteroaryl ring having 1-4 heteroatoms independently selected from nitrogen, oxygen, and sulfur; wherein R^{8A} is substituted by z² instances of R^{2C}.

In some embodiments, R^{8A} is phenyl-R^{CyB}; naphthyl-R^{CyB}; an 8-10 membered bicyclic heteroaryl ring (substituted with —R^{CyB}) having 1-4 heteroatoms independently selected from nitrogen, oxygen, and sulfur; or a 7-12 membered saturated or partially unsaturated bicyclic heterocyclic ring (substituted with —R^{CyB}) having 1-4 heteroatoms independently selected from nitrogen, oxygen, and sulfur; wherein R^{8A} is substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{8A} is phenyl-R^{CyB}; an 8-10 membered bicyclic heteroaryl ring (substituted with —R^{CyB}) having 1-4 heteroatoms independently selected from nitrogen, oxygen, and sulfur; or a 7-12 membered saturated or partially unsaturated bicyclic heterocyclic ring (substituted with —R^{CyB}) having 1-4 heteroatoms independently selected from nitrogen, oxygen, and sulfur; wherein R^{8A} is substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{8A} is phenyl-R^{CyB} or an 8-10 membered bicyclic heteroaryl ring (substituted with —R^{CyB}) having 1-4 heteroatoms independently selected from nitrogen, oxygen, and sulfur; wherein R^{8A} is substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{8A} is phenyl-R^{CyB} substituted by z² instances of R^{2C}. In some embodiments, R^{8A} is phenyl-R^{CyB} substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{8A} is phenyl-R^{CyB} substituted by 1-3 instances of a group independently selected from halogen, —CN, —O-(optionally substituted C₁₋₆ aliphatic), and an optionally substituted C₁₋₆ aliphatic. In some embodiments, R^{8A} is phenyl-R^{CyB} substituted by 1-3 instances of a group independently selected from halogen and C₁₋₃ aliphatic optionally substituted with 1-3 halogen. In some embodiments, R^{8A} is phenyl-R^{CyB} substituted by 1-3 instances of a group independently selected from fluorine, chlorine, —CH₃, —CHF₂, and —CF₃.

In some embodiments, R^{8A} is phenyl-R^{CyB} substituted by 2 instances of a group independently selected from halogen, —CN, —O-(optionally substituted C₁₋₆ aliphatic), and an optionally substituted C₁₋₆ aliphatic. In some embodiments, R^{8A} is phenyl-R^{CyB} substituted by 2 instances of a group independently selected from halogen and C₁₋₃ aliphatic optionally substituted with 1-3 halogen. In some embodiments, R^{8A} is phenyl substituted by 2 instances of a group independently selected from fluorine, chlorine, —CH₃, —CHF₂, and —CF₃.

In some embodiments, R^{8A} is an 8-10 membered bicyclic heteroaryl ring (substituted with —R^{CyB}) having 1-4 heteroatoms independently selected from nitrogen, oxygen, and sulfur; wherein R^{8A} is substituted by z² instances of R^{2C}. In some embodiments, R^{8A} is an 8-10 membered bicyclic heteroaryl ring (substituted with —R^{CyB}) having 1-4 heteroatoms independently selected from nitrogen, oxygen, and sulfur; wherein R^{8A} is substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{8A} is an 8-10 membered bicyclic heteroaryl ring (substituted with —R^{CyB}) having 1-2 heteroatoms independently selected from nitrogen, oxygen, and sulfur; wherein R^{8A} is substituted by z² instances of R^{2C}. In some embodiments, R^{8A} is an 8-10 membered bicyclic heteroaryl ring (substituted with —R^{CyB}) having 1-2 heteroatoms independently selected from nitrogen, oxygen, and sulfur; wherein R^{8A} is substituted by z² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{8A} is an 8-10 membered bicyclic heteroaryl ring (substituted with —R^{CyB}) having 1-2 heteroatoms independently selected from nitrogen, oxygen, and sulfur; wherein R^{8A} is substituted by 0-2 instances of a group independently selected from halogen, —CN, —O-(optionally substituted C₁₋₆ aliphatic), and an optionally substituted C₁₋₆ aliphatic. In some embodiments, R^{8A} is an 8-10 membered bicyclic heteroaryl ring (substituted with —R^{CyB}) having 1-2 heteroatoms independently selected from nitrogen, oxygen, and sulfur; wherein R^{8A} is substituted by 0-2 instances of a group independently selected from halogen and C₁₋₃ aliphatic optionally substituted with 1-3 halogen. In some embodiments, R^{8A} is an 8-10 membered bicyclic heteroaryl ring (substituted with —R^{CyB}) having 1-2 heteroatoms independently selected from nitrogen, oxygen, and sulfur; wherein R^{8A} is substituted by 0-2 instances of a group independently selected from fluorine, chlorine, —CN, —CH₃, —CHF₂, and —CF₃.

In some embodiments, R^{8A} is:

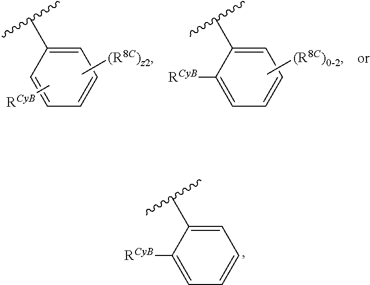

wherein R^{CyB}, R^{2C} and z² are as defined in the embodiments and classes and subclasses herein. In some embodiments, R^{8A} is

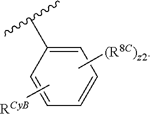

In some embodiments, R^{8A} is

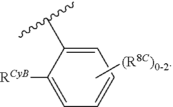

In some embodiments, R^{8A} is

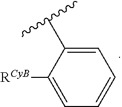

In some embodiments, R^{8A} is R^{8C}

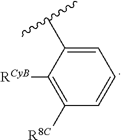

In some embodiments, R^{8A} is

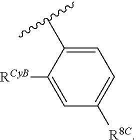

In some embodiments, R^{8A} is

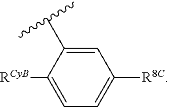

In some embodiments, R^{8A} is

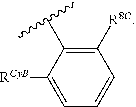

In some embodiments, R^{8A} is

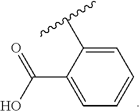 [SMILES: *c1ccccc1C(=O)O]

n some embodiments, R^{8A} is

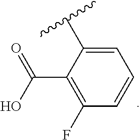 [SMILES: *c1cccc(F)c1C(=O)O]

In some embodiments, R^{8A} is

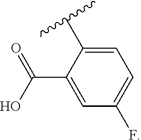 [SMILES: *c1ccc(F)cc1C(=O)O]

In some embodiments, R^{8A} is

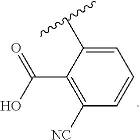 [SMILES: *c1cccc(C#N)c1C(=O)O]

In some embodiments, R^{8A} is

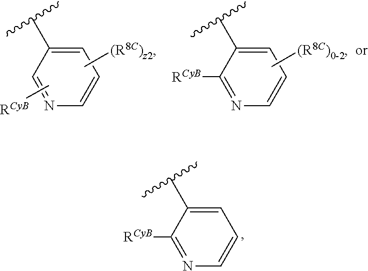

wherein R^{CyB}, R^{2C} and z² are as defined in the embodiments and classes and subclasses herein. In some embodiments, R^{8A} is

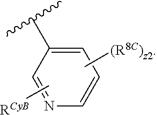

In some embodiments, R^{8A} is

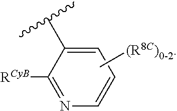

In some embodiments, R^{8A} is

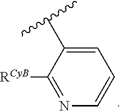

In some embodiments, R^{8A} is

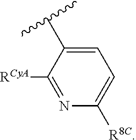

In some embodiments, R^{8A} is

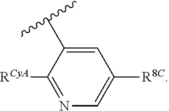

In some embodiments, R^{8A} is

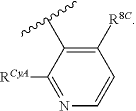

In some embodiments, R^{8A} is

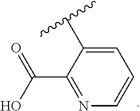 [SMILES: *c1cccnc1C(=O)O]

In some embodiments, R^{8A} is

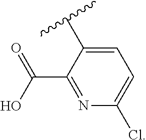 [SMILES: *c1ccc(Cl)nc1C(=O)O]

In some embodiments, R^{8A} is selected from the groups depicted in the compounds in Table 1.

As defined generally above, R^{G1A} is R^{J} or R^{K} substituted by z³ instances of R^{G1C}. In some embodiments, R^{G1A} is R^{J}. In some embodiments, R^{G1A} is R^{K} substituted by z³ instances of R^{G1C}.

In some embodiments, R^{G1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{G1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G1A} is oxo. In some embodiments, R^{G1A} is halogen. In some embodiments, R^{G1A} is —CN. In some embodiments, R^{G1A} is —NO₂. In some embodiments, R^{G1A} is —OR. In some embodiments, R^{G1A} is —SR. In some embodiments, R^{G1A} is —NR₂. In some embodiments, R^{G1A} is —S(O)₂R. In some embodiments, R^{G1A} is —S(O)₂NR₂. In some embodiments, R^{G1A} is —S(O)₂F. In some embodiments, R^{G1A} is —S(O)R. In some embodiments, R^{G1A} is —S(O)NR₂. In some embodiments, R^{G1A} is —S(O)(NR)R. In some embodiments, R^{G1A} is —C(O)R. In some embodiments, R^{G1A} is —C(O)OR. In some embodiments, R^{G1A} is —C(O)NR₂. In some embodiments, R^{G1A} is —C(O)N(R)OR. In some embodiments, R^{G1A} is —OC(O)R. In some embodiments, R^{G1A} is —OC(O)NR₂. In some embodiments, R^{G1A} is —N(R)C(O)OR. In some embodiments, R^{G1A} is —N(R)C(O)R. In some embodiments, R^{G1A} is —N(R)C(O)NR₂. In some embodiments, R^{G1A} is —N(R)C(NR)NR₂. In some embodiments, R^{G1A} is —N(R)S(O)₂NR₂. In some embodiments, R^{G1A} is —N(R)S(O)₂R. In some embodiments, R^{G1A} is —P(O)R₂. In some embodiments, R^{G1A} is —P(O)(R)OR. In some embodiments, R^{G1A} is —B(OR)₂. In some embodiments, R^{G1A} is deuterium.

In some embodiments, R^{G1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G1A} is halogen, —CN, or —NO₂. In some embodiments, R^{G1A} is —OR, —SR, or —NR₂. In some embodiments, R^{G1A} is —S(O)₂R, —S(O)₂NR₂, —S(O)₂F, —S(O)R, —S(O)NR₂, or —S(O)(NR)R. In some embodiments, R^{G1A} is —C(O)R, —C(O)OR, —C(O)NR₂, or —C(O)N(R)OR. In some embodiments, R^{G1A} is —OC(O)R or —OC(O)NR₂. In some embodiments, R^{G1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{G1A} is —P(O)R₂ or —P(O)(R)OR.

In some embodiments, R^{G1A} is —OR, —OC(O)R, or —OC(O)NR₂. In some embodiments, R^{G1A} is —SR, —S(O)₂R, —S(O)₂NR₂, —S(O)₂F, —S(O)R, —S(O)NR₂, or —S(O)(NR)R. In some embodiments, R^{G1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{G1A} is —S(O)₂R, —S(O)₂NR₂, or —S(O)₂F. In some embodiments, R^{G1A} is —S(O)R, —S(O)NR₂, or —S(O)(NR)R. In some embodiments, R^{G1A} is —SR, —S(O)₂R, or —S(O)R. In some embodiments, R^{G1A} is —S(O)₂NR₂, —S(O)NR₂, or —S(O)(NR)R. In some embodiments, R^{G1A} is —S(O)₂NR₂ or —S(O)NR₂. In some embodiments, R^{G1A} is —SR, —S(O)₂R, —S(O)₂NR₂, or —S(O)R.

In some embodiments, R^{G1A} is —N(R)C(O)OR, —N(R)C(O)R, or —N(R)C(O)NR₂. In some embodiments, R^{G1A} is —N(R)S(O)₂NR₂ or —N(R)S(O)₂R. In some embodiments, R^{G}A is —N(R)C(O)OR or —N(R)C(O)R. In some embodiments, R^{G1A} is —N(R)C(O)NR₂ or —N(R)S(O)₂NR₂. In some embodiments, R^{G1A} is —N(R)C(O)OR, —N(R)C(O)R, or —N(R)S(O)₂R.

In some embodiments, R^{G1A} is —NR₂, —N(R)C(O)OR, —N(R)C(O)R, or —N(R)C(O)NR₂. In some embodiments, R^{G1A} is —NR₂, —N(R)C(O)OR, or —N(R)C(O)R. In some embodiments, R^{G1A} is —NR₂, —N(R)C(O)OR, —N(R)C(O)R, or —N(R)S(O)₂R.

In some embodiments, R^{G1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is a C₁₋₆ aliphatic chain substituted by z³ instances of R^{G1C}. In some embodiments, R^{G1A} is phenyl substituted by z³ instances of R^{G1C}. In some embodiments, R^{G1A} is naphthyl substituted by z³ instances of R^{G1C}. In some embodiments, R^{G1A} is a 5-6 membered monocyclic heteroaryl ring having 1-4 heteroatoms independently selected from nitrogen, oxygen, and sulfur; wherein said ring is substituted by z³ instances of R^{G1C}. In some embodiments, R^{G1A} is an 8-10 membered bicyclic heteroaryl ring having 1-4 heteroatoms independently selected from nitrogen, oxygen, and sulfur; wherein said ring is substituted by z³ instances of R^{G1C}. In some embodiments, R^{G1A} is a 3-7 membered saturated or partially unsaturated monocyclic carbocyclic ring substituted by z³ instances of R^{G1C}. In some embodiments, R^{G1A} is a 5-12 membered saturated or partially unsaturated bicyclic carbocyclic ring substituted by z³ instances of R^{G1C}. In some embodiments, R^{G1A} is a 3-7 membered saturated or partially unsaturated monocyclic heterocyclic ring having 1-2 heteroatoms independently selected from nitrogen, oxygen, and sulfur; wherein said ring is substituted by z³ instances of R^{G1C}. In some embodiments, R^{G1A} is a 7-12 membered saturated or partially unsaturated bicyclic heterocyclic ring having 1-4 heteroatoms independently selected from nitrogen, oxygen, and sulfur; wherein said ring is substituted by z³ instances of R^{G1C}.

In some embodiments, R^{G1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z³ instances of R^{G1C}. In some embodiments, R^{G1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z³ instances of R^{G1C}. In some embodiments, R^{G1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z³ instances of R^{G1C}. In some embodiments, R^{G1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl or naphthyl; each of which is substituted by z³ instances of R^{G1C}. In some embodiments, R^{G1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z³ instances of R^{G1C}. In some embodiments, R^{G1A} is a 3-7 membered saturated or partially unsaturated monocyclic carbocyclic ring or a 5-12 membered saturated or partially unsaturated bicyclic carbocyclic ring; each of which is substituted by z³ instances of R^{G1C}. In some embodiments, R^{G1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl or a 5-6 membered monocyclic heteroaryl ring having 1-4 heteroatoms independently selected from nitrogen, oxygen, and sulfur; each of which is substituted by z³ instances of R^{G1C}. In some embodiments, R^{G1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z³ instances of R^{G1C}. In some embodiments, R^{G1A} is naphthyl or an 8-10 membered bicyclic heteroaryl ring having 1-4 heteroatoms independently selected from nitrogen, oxygen, and sulfur; each of which is substituted by z³ instances of R^{G1C}. In some embodiments, R^{G1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is phenyl or a 3-7 membered saturated or partially unsaturated monocyclic carbocyclic ring; each of which is substituted by z³ instances of R^{G1C} In some embodiments, R^{G1A} is naphthyl or a 5-12 membered saturated or partially unsaturated bicyclic carbocyclic ring; each of which is substituted by z³ instances of R^{G1C}. In some embodiments, R^{G1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by z³ instances of R^{G1C}. In some embodiments, R^{G1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}.

In some embodiments, R^{G1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z³ instances of R^{G1C}. In some embodiments, R^{G1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z³ instances of R^{G1C}. In some embodiments, R^{G1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z³ instances of R^{G1C}.

In some embodiments, R^{G1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z³ instances of R^{G1C}. In some embodiments, R^{G1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z³ instances of R^{G1C}. In some embodiments, R^{G1A} is a C₁₋₆ aliphatic chain, phenyl, or a 3-7 membered saturated or partially unsaturated monocyclic carbocyclic ring; each of which is substituted by z³ instances of R^{G1C}.

In some embodiments, R^{G1A} is selected from the groups depicted in the compounds in Table 1.

As defined generally above, R^{G2A} is R^{J} or R^{K} substituted by z⁴ instances of R^{G2C}. In some embodiments, R^{G2A} is R^{J}. In some embodiments, R^{G2A} is R^{K} substituted by z⁴ instances of R^{G2C}. In some embodiments, R^{G2A} is a C₁₋₆ aliphatic chain or halogen.

In some embodiments, R^{G2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{G2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G2A} is oxo. In some embodiments, R^{G2A} is halogen. In some embodiments, R^{G2A} is —CN. In some embodiments, R^{G2A} is —NO₂. In some embodiments, R^{G2A} is —OR. In some embodiments, R^{G2A} is —SR. In some embodiments, R^{G2A} is —NR₂. In some embodiments, R^{G2A} is —S(O)₂R. In some embodiments, R^{G2A} is —S(O)₂NR₂. In some embodiments, R^{G2A} is —S(O)₂F. In some embodiments, R^{G2A} is —S(O)R. In some embodiments, R^{G2A} is —S(O)NR₂. In some embodiments, R^{G2A} is —S(O)(NR)R. In some embodiments, R^{G2A} is —C(O)R. In some embodiments, R^{G2A} is —C(O)OR. In some embodiments, R^{G2A} is —C(O)NR₂. In some embodiments, R^{G2A} is —C(O)N(R)OR. In some embodiments, R^{G2A} is —OC(O)R. In some embodiments, R^{G2A} is —OC(O)NR₂. In some embodiments, R^{G2A} is —N(R)C(O)OR. In some embodiments, R^{G2A} is —N(R)C(O)R. In some embodiments, R^{G2A} is —N(R)C(O)NR₂. In some embodiments, R^{G2A} is —N(R)C(NR)NR₂. In some embodiments, R^{G2A} is —N(R)S(O)₂NR₂. In some embodiments, R^{G2A} is —N(R)S(O)₂R. In some embodiments, R^{G2A} is —P(O)R₂. In some embodiments, R^{G2A} is —P(O)(R)OR. In some embodiments, R^{G2A} is —B(OR)₂. In some embodiments, R^{G2A} is deuterium.

In some embodiments, R^{G2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G2A} is halogen, —CN, or —NO₂. In some embodiments, R^{G2A} is —OR, —SR, or —NR₂. In some embodiments, R^{G2A} is —S(O)₂R, —S(O)₂NR₂, —S(O)₂F, —S(O)R, —S(O)NR₂, or —S(O)(NR)R. In some embodiments, R^{G2A} is —C(O)R, —C(O)OR, —C(O)NR₂, or —C(O)N(R)OR. In some embodiments, R^{G2A} is —OC(O)R or —OC(O)NR₂. In some embodiments, R^{G2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{G2A} is —P(O)R₂ or —P(O)(R)OR.

In some embodiments, R^{G2A} is —OR, —OC(O)R, or —OC(O)NR₂. In some embodiments, R^{G2A} is —SR, —S(O)₂R, —S(O)₂NR₂, —S(O)₂F, —S(O)R, —S(O)NR₂, or —S(O)(NR)R. In some embodiments, R^{G2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{G2A} is —S(O)₂R, —S(O)₂NR₂, or —S(O)₂F. In some embodiments, R^{G2A} is —S(O)R, —S(O)NR₂, or —S(O)(NR)R. In some embodiments, R^{G2A} is —SR, —S(O)₂R, or —S(O)R. In some embodiments, R^{G2A} is —S(O)₂NR₂, —S(O)NR₂, or —S(O)(NR)R. In some embodiments, R^{G2A} is —S(O)₂NR₂ or —S(O)NR₂. In some embodiments, R^{G2A} is —SR, —S(O)₂R, —S(O)₂NR₂, or —S(O)R.

In some embodiments, R^{G2A} is —N(R)C(O)OR, —N(R)C(O)R, or —N(R)C(O)NR₂. In some embodiments, R^{G2A} is —N(R)S(O)₂NR₂ or —N(R)S(O)₂R. In some embodiments, R^{G2A} is —N(R)C(O)OR or —N(R)C(O)R. In some embodiments, R^{G2A} is —N(R)C(O)NR₂ or —N(R)S(O)₂NR₂. In some embodiments, R^{G2A} is —N(R)C(O)OR, —N(R)C(O)R, or —N(R)S(O)₂R.

In some embodiments, R^{G2A} is —NR₂, —N(R)C(O)OR, —N(R)C(O)R, or —N(R)C(O)NR₂. In some embodiments, R^{G2A} is —NR₂, —N(R)C(O)OR, or —N(R)C(O)R. In some embodiments, R^{G2A} is —NR₂, —N(R)C(O)OR, —N(R)C(O)R, or —N(R)S(O)₂R.

In some embodiments, R^{G2A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is a C₁₋₆ aliphatic chain substituted by z⁴ instances of R^{G2C}. In some embodiments, R^{G2A} is phenyl substituted by z⁴ instances of R^{G2C}. In some embodiments, R^{G2A} is naphthyl substituted by z⁴ instances of R^{G2C}. In some embodiments, R^{G2A} is a 5-6 membered monocyclic heteroaryl ring having 1-4 heteroatoms independently selected from nitrogen, oxygen, and sulfur; wherein said ring is substituted by z⁴ instances of R^{G2C}. In some embodiments, R^{G2A} is an 8-10 membered bicyclic heteroaryl ring having 1-4 heteroatoms independently selected from nitrogen, oxygen, and sulfur; wherein said ring is substituted by z⁴ instances of R^{G2C}. In some embodiments, R^{G2A} is a 3-7 membered saturated or partially unsaturated monocyclic carbocyclic ring substituted by z⁴ instances of R^{G2C}. In some embodiments, R^{G2A} is a 5-12 membered saturated or partially unsaturated bicyclic carbocyclic ring substituted by z⁴ instances of R^{G2C}. In some embodiments, R^{G2A} is a 3-7 membered saturated or partially unsaturated monocyclic heterocyclic ring having 1-2 heteroatoms independently selected from nitrogen, oxygen, and sulfur; wherein said ring is substituted by z⁴ instances of R^{G2C}. In some embodiments, R^{G2A} is a 7-12 membered saturated or partially unsaturated bicyclic heterocyclic ring having 1-4 heteroatoms independently selected from nitrogen, oxygen, and sulfur; wherein said ring is substituted by z⁴ instances of R^{G2C}.

In some embodiments, R^{G2A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁴ instances of R^{G2C}. In some embodiments, R^{G2A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z⁴ instances of R^{G2C}. In some embodiments, R^{G2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁴ instances of R^{G2C}. In some embodiments, R^{G2A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl or naphthyl; each of which is substituted by z⁴ instances of R^{G2C}. In some embodiments, R^{G2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁴ instances of R^{G2C}. In some embodiments, R^{G2A} is a 3-7 membered saturated or partially unsaturated monocyclic carbocyclic ring or a 5-12 membered saturated or partially unsaturated bicyclic carbocyclic ring; each of which is substituted by z⁴ instances of R^{G2C}. In some embodiments, R^{G2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl or a 5-6 membered monocyclic heteroaryl ring having 1-4 heteroatoms independently selected from nitrogen, oxygen, and sulfur; each of which is substituted by z⁴ instances of R^{G2C}. In some embodiments, R^{G2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z⁴ instances of R^{G2C}. In some embodiments, R^{G2A} is naphthyl or an 8-10 membered bicyclic heteroaryl ring having 1-4 heteroatoms independently selected from nitrogen, oxygen, and sulfur; each of which is substituted by z⁴ instances of R^{G2C}. In some embodiments, R^{G2A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is phenyl or a 3-7 membered saturated or partially unsaturated monocyclic carbocyclic ring; each of which is substituted by z⁴ instances of R^{G2C} In some embodiments, R^{G2A} is naphthyl or a 5-12 membered saturated or partially unsaturated bicyclic carbocyclic ring; each of which is substituted by z⁴ instances of R^{G2C}. In some embodiments, R^{G2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r3 instances of R^{G2C}. In some embodiments, R^{G2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}.

In some embodiments, R^{G2A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁴ instances of R^{G2C}. In some embodiments, R^{G2A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z⁴ instances of R^{G2C}. In some embodiments, R^{G2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁴ instances of R^{G2C}.

In some embodiments, R^{G2A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z⁴ instances of R^{G2C}. In some embodiments, R^{G2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z⁴ instances of R^{G2C}. In some embodiments, R^{G2A} is a C₁₋₆ aliphatic chain, phenyl, or a 3-7 membered saturated or partially unsaturated monocyclic carbocyclic ring; each of which is substituted by z⁴ instances of R^{G2C}.

In some embodiments, R^{G2A} is selected from the groups depicted in the compounds in Table 1.

As defined generally above, R^{G3A} is R^{J} or R^{K} substituted by z⁵ instances of R^{G3C}. In some embodiments, R^{G3A} is R^{J}. In some embodiments, R^{G3A} is R^{K} substituted by z⁵ instances of R^{G3C}.

In some embodiments, R^{G3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{G3A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G3A} is oxo. In some embodiments, R^{G3A} is halogen. In some embodiments, R^{G3A} is —CN. In some embodiments, R^{G3A} is —NO₂. In some embodiments, R^{G3A} is —OR. In some embodiments, R^{G3A} is —SR. In some embodiments, R^{G3A} is —NR₂. In some embodiments, R^{G3A} is —S(O)₂R. In some embodiments, R^{G3A} is —S(O)₂NR₂. In some embodiments, R^{G3A} is —S(O)₂F. In some embodiments, R^{G3A} is —S(O)R. In some embodiments, R^{G3A} is —S(O)NR₂. In some embodiments, R^{G3A} is —S(O)(NR)R. In some embodiments, R^{G3A} is —C(O)R. In some embodiments, R^{G3A} is —C(O)OR. In some embodiments, R^{G3A} is —C(O)NR₂. In some embodiments, R^{G3A} is —C(O)N(R)OR. In some embodiments, R^{G3A} is —OC(O)R. In some embodiments, R^{G3A} is —OC(O)NR₂. In some embodiments, R^{G3A} is —N(R)C(O)OR. In some embodiments, R^{G3A} is —N(R)C(O)R. In some embodiments, R^{G3A} is —N(R)C(O)NR₂. In some embodiments, R^{G3A} is —N(R)C(NR)NR₂. In some embodiments, R^{G3A} is —N(R)S(O)₂NR₂. In some embodiments, R^{G3A} is —N(R)S(O)₂R. In some embodiments, R^{G3A} is —P(O)R₂. In some embodiments, R^{G3A} is —P(O)(R)OR. In some embodiments, R^{G3A} is —B(OR)₂. In some embodiments, R^{G3A} is deuterium.

In some embodiments, R^{G3A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{G3A} is halogen, —CN, or —NO₂. In some embodiments, R^{G3A} is —OR, —SR, or —NR₂. In some embodiments, R^{G3A} is —S(O)₂R, —S(O)₂NR₂, —S(O)₂F, —S(O)R, —S(O)NR₂, or —S(O)(NR)R. In some embodiments, R^{G3A} is —C(O)R, —C(O)OR, —C(O)NR₂, or —C(O)N(R)OR. In some embodiments, R^{G3A} is —OC(O)R or —OC(O)NR₂. In some embodiments, R^{G3A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{G3A} is —P(O)R₂ or —P(O)(R)OR.

In some embodiments, R^{G3A} is —OR, —OC(O)R, or —OC(O)NR₂. In some embodiments, R^{G3A} is —SR, —S(O)₂R, —S(O)₂NR₂, —S(O)₂F, —S(O)R, —S(O)NR₂, or —S(O)(NR)R. In some embodiments, R^{G3A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{G3A} is —S(O)₂R, —S(O)₂NR₂, or —S(O)₂F. In some embodiments, R^{G3A} is —S(O)R, —S(O)NR₂, or —S(O)(NR)R. In some embodiments, R^{G3A} is —SR, —S(O)₂R, or —S(O)R. In some embodiments, R^{G3A} is —S(O)₂NR₂, —S(O)NR₂, or —S(O)(NR)R. In some embodiments, R^{G3A} is —S(O)₂NR₂ or —S(O)NR₂. In some embodiments, R^{G3A} is —SR, —S(O)₂R, —S(O)₂NR₂, or —S(O)R.

In some embodiments, R^{G3A} is —N(R)C(O)OR, —N(R)C(O)R, or —N(R)C(O)NR₂. In some embodiments, R^{G3A} is —N(R)S(O)₂NR₂ or —N(R)S(O)₂R. In some embodiments, R^{G3A} is —N(R)C(O)OR or —N(R)C(O)R. In some embodiments, R^{G3A} is —N(R)C(O)NR₂ or —N(R)S(O)₂NR₂. In some embodiments, R^{G3A} is —N(R)C(O)OR, —N(R)C(O)R, or —N(R)S(O)₂R.

In some embodiments, R^{G3A} is —NR₂, —N(R)C(O)OR, —N(R)C(O)R, or —N(R)C(O)NR₂. In some embodiments, R^{G3A} is —NR₂, —N(R)C(O)OR, or —N(R)C(O)R. In some embodiments, R^{G3A} is —NR₂, —N(R)C(O)OR, —N(R)C(O)R, or —N(R)S(O)₂R.

In some embodiments, R^{G3A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is a C₁₋₆ aliphatic chain substituted by z⁵ instances of R^{G3C}. In some embodiments, R^{G3A} is phenyl substituted by z⁵ instances of R^{G3C}. In some embodiments, R^{G3A} is naphthyl substituted by z⁵ instances of R^{G3C}. In some embodiments, R^{G3A} is a 5-6 membered monocyclic heteroaryl ring having 1-4 heteroatoms independently selected from nitrogen, oxygen, and sulfur; wherein said ring is substituted by z⁵ instances of R^{G3C}. In some embodiments, R^{G3A} is an 8-10 membered bicyclic heteroaryl ring having 1-4 heteroatoms independently selected from nitrogen, oxygen, and sulfur; wherein said ring is substituted by z⁵ instances of R^{G3C}. In some embodiments, R^{G3A} is a 3-7 membered saturated or partially unsaturated monocyclic carbocyclic ring substituted by z⁵ instances of R^{G3C}. In some embodiments, R^{G3A} is a 5-12 membered saturated or partially unsaturated bicyclic carbocyclic ring substituted by z⁵ instances of R^{G3C}. In some embodiments, R^{G3A} is a 3-7 membered saturated or partially unsaturated monocyclic heterocyclic ring having 1-2 heteroatoms independently selected from nitrogen, oxygen, and sulfur; wherein said ring is substituted by z⁵ instances of R^{G3C}. In some embodiments, R^{G3A} is a 7-12 membered saturated or partially unsaturated bicyclic heterocyclic ring having 1-4 heteroatoms independently selected from nitrogen, oxygen, and sulfur; wherein said ring is substituted by z⁵ instances of R^{G3C}.

In some embodiments, R^{G3A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁵ instances of R^{G3C}. In some embodiments, R^{G3A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z⁵ instances of R^{G3C}. In some embodiments, R^{G3A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁵ instances of R^{G3C}. In some embodiments, R^{G3A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl or naphthyl; each of which is substituted by z⁵ instances of R^{G3C}. In some embodiments, R^{G3A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁵ instances of R^{G3C}. In some embodiments, R^{G3A} is a 3-7 membered saturated or partially unsaturated monocyclic carbocyclic ring or a 5-12 membered saturated or partially unsaturated bicyclic carbocyclic ring; each of which is substituted by z⁵ instances of R^{G3C}. In some embodiments, R^{G3A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl or a 5-6 membered monocyclic heteroaryl ring having 1-4 heteroatoms independently selected from nitrogen, oxygen, and sulfur; each of which is substituted by z⁵ instances of R^{G3C}. In some embodiments, R^{G3A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z⁵ instances of R^{G3C}. In some embodiments, R^{G3A} is naphthyl or an 8-10 membered bicyclic heteroaryl ring having 1-4 heteroatoms independently selected from nitrogen, oxygen, and sulfur; each of which is substituted by z⁵ instances of R^{G3C}. In some embodiments, R^{G3A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is phenyl or a 3-7 membered saturated or partially unsaturated monocyclic carbocyclic ring; each of which is substituted by z⁵ instances of R^{G3C} In some embodiments, R^{G3A} is naphthyl or a 5-12 membered saturated or partially unsaturated bicyclic carbocyclic ring; each of which is substituted by z⁵ instances of R^{G3C}. In some embodiments, R^{G3A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r3 instances of R^{G3C}. In some embodiments, R^{G3A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}.

In some embodiments, R^{G3A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁵ instances of R^{G3C}. In some embodiments, R^{G3A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z⁵ instances of R^{G3C}. In some embodiments, R^{G3A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁵ instances of R^{G3C}.

In some embodiments, R^{G3A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z⁵ instances of R^{G3C}. In some embodiments, R^{G3A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z⁵ instances of R^{G3C}. In some embodiments, R^{G3A} is a C₁₋₆ aliphatic chain, phenyl, or a 3-7 membered saturated or partially unsaturated monocyclic carbocyclic ring; each of which is substituted by z⁵ instances of R^{G3C}.

In some embodiments, R^{G3A} is selected from the groups depicted in the compounds in Table 1.

As defined generally above, R^{M1A} is R^{J} or R^{K} substituted by z⁶ instances of R^{M1C}. In some embodiments, R^{M1A} is R^{J}. In some embodiments, R^{M1A} is R^{K} substituted by z⁶ instances of R^{M1C}. In some embodiments, R^{M1A} is a C₁₋₆ aliphatic chain or phenyl substituted by z⁶ instances of R^{M1C}.

In some embodiments, R^{M1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{M1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M1A} is oxo. In some embodiments, R^{M1A} is halogen. In some embodiments, R^{M1A} is —CN. In some embodiments, R^{M1A} is —NO₂. In some embodiments, R^{M1A} is —OR. In some embodiments, R^{M1A} is —SR. In some embodiments, R^{M1A} is —NR₂. In some embodiments, R^{M1A} is —S(O)₂R. In some embodiments, R^{M1A} is —S(O)₂NR₂. In some embodiments, R^{M1A} is —S(O)₂F. In some embodiments, R^{M1A} is —S(O)R. In some embodiments, R^{M1A} is —S(O)NR₂. In some embodiments, R^{M1A} is —S(O)(NR)R. In some embodiments, R^{M1A} is —C(O)R. In some embodiments, R^{M1A} is —C(O)OR. In some embodiments, R^{M1A} is —C(O)NR₂. In some embodiments, R^{M1A} is —C(O)N(R)OR. In some embodiments, R^{M1A} is —OC(O)R. In some embodiments, R^{M1A} is —OC(O)NR₂. In some embodiments, R^{M1A} is —N(R)C(O)OR. In some embodiments, R^{M1A} is —N(R)C(O)R. In some embodiments, R^{M1A} is —N(R)C(O)NR₂. In some embodiments, R^{M1A} is —N(R)C(NR)NR₂. In some embodiments, R^{M1A} is —N(R)S(O)₂NR₂. In some embodiments, R^{M1A} is —N(R)S(O)₂R. In some embodiments, R^{M1A} is —P(O)R₂. In some embodiments, R^{M1A} is —P(O)(R)OR. In some embodiments, R^{M1A} is —B(OR)₂. In some embodiments, R^{M1A} is deuterium.

In some embodiments, R^{M1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M1A} is halogen, —CN, or —NO₂. In some embodiments, R^{M1A} is —OR, —SR, or —NR₂. In some embodiments, R^{M1A} is —S(O)₂R, —S(O)₂NR₂, —S(O)₂F, —S(O)R, —S(O)NR₂, or —S(O)(NR)R. In some embodiments, R^{M1A} is —C(O)R, —C(O)OR, —C(O)NR₂, or —C(O)N(R)OR. In some embodiments, R^{M1A} is —OC(O)R or —OC(O)NR₂. In some embodiments, R^{M1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{M1A} is —P(O)R₂ or —P(O)(R)OR.

In some embodiments, R^{M1A} is —OR, —OC(O)R, or —OC(O)NR₂. In some embodiments, R^{M1A} is —SR, —S(O)₂R, —S(O)₂NR₂, —S(O)₂F, —S(O)R, —S(O)NR₂, or —S(O)(NR)R. In some embodiments, R^{M1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{M1A} is —S(O)₂R, —S(O)₂NR₂, or —S(O)₂F. In some embodiments, R^{M1A} is —S(O)R, —S(O)NR₂, or —S(O)(NR)R. In some embodiments, R^{M1A} is —SR, —S(O)₂R, or —S(O)R. In some embodiments, R^{M1A} is —S(O)₂NR₂, —S(O)NR₂, or —S(O)(NR)R. In some embodiments, R^{M1A} is —S(O)₂NR₂ or —S(O)NR₂. In some embodiments, R^{M1A} is —SR, —S(O)₂R, —S(O)₂NR₂, or —S(O)R.

In some embodiments, R^{M1A} is —N(R)C(O)OR, —N(R)C(O)R, or —N(R)C(O)NR₂. In some embodiments, R^{M1A} is —N(R)S(O)₂NR₂ or —N(R)S(O)₂R. In some embodiments, R^{M1A} is —N(R)C(O)OR or —N(R)C(O)R. In some embodiments, R^{M1A} is —N(R)C(O)NR₂ or —N(R)S(O)₂NR₂. In some embodiments, R^{M1A} is —N(R)C(O)OR, —N(R)C(O)R, or —N(R)S(O)₂R.

In some embodiments, R^{M1A} is —NR₂, —N(R)C(O)OR, —N(R)C(O)R, or —N(R)C(O)NR₂. In some embodiments, R^{M1A} is —NR₂, —N(R)C(O)OR, or —N(R)C(O)R. In some embodiments, R^{M1A} is —NR₂, —N(R)C(O)OR, —N(R)C(O)R, or —N(R)S(O)₂R.

In some embodiments, R^{M1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is a C₁₋₆ aliphatic chain substituted by z⁶ instances of R^{M1C}. In some embodiments, R^{M1A} is phenyl substituted by z⁶ instances of R^{M1C}. In some embodiments, R^{M1A} is naphthyl substituted by z⁶ instances of R^{M1C}. In some embodiments, R^{M1A} is a 5-6 membered monocyclic heteroaryl ring having 1-4 heteroatoms independently selected from nitrogen, oxygen, and sulfur; wherein said ring is substituted by z⁶ instances of R^{M1C}. In some embodiments, R^{M1A} is an 8-10 membered bicyclic heteroaryl ring having 1-4 heteroatoms independently selected from nitrogen, oxygen, and sulfur; wherein said ring is substituted by z⁶ instances of R^{M1C}. In some embodiments, R^{M1A} is a 3-7 membered saturated or partially unsaturated monocyclic carbocyclic ring substituted by z⁶ instances of R^{M1C}. In some embodiments, R^{M1A} is a 5-12 membered saturated or partially unsaturated bicyclic carbocyclic ring substituted by z⁶ instances of R^{M1C}. In some embodiments, R^{M1A} is a 3-7 membered saturated or partially unsaturated monocyclic heterocyclic ring having 1-2 heteroatoms independently selected from nitrogen, oxygen, and sulfur; wherein said ring is substituted by z⁶ instances of R^{M1C}. In some embodiments, R^{M1A} is a 7-12 membered saturated or partially unsaturated bicyclic heterocyclic ring having 1-4 heteroatoms independently selected from nitrogen, oxygen, and sulfur; wherein said ring is substituted by z⁶ instances of R^{M1C}.

In some embodiments, R^{M1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁶ instances of R^{M1C}. In some embodiments, R^{M1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z⁶ instances of R^{M1C}. In some embodiments, R^{M1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁶ instances of R^{M1C}. In some embodiments, R^{M1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl or naphthyl; each of which is substituted by z⁶ instances of R^{M1C}. In some embodiments, R^{M1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁶ instances of R^{M1C}. In some embodiments, R^{M1A} is a 3-7 membered saturated or partially unsaturated monocyclic carbocyclic ring or a 5-12 membered saturated or partially unsaturated bicyclic carbocyclic ring; each of which is substituted by z⁶ instances of R^{M1C}. In some embodiments, R^{M1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl or a 5-6 membered monocyclic heteroaryl ring having 1-4 heteroatoms independently selected from nitrogen, oxygen, and sulfur; each of which is substituted by z⁶ instances of R^{M1C}. In some embodiments, R^{M1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z⁶ instances of R^{M1C}. In some embodiments, R^{M1A} is naphthyl or an 8-10 membered bicyclic heteroaryl ring having 1-4 heteroatoms independently selected from nitrogen, oxygen, and sulfur; each of which is substituted by z⁶ instances of R^{M1C}. In some embodiments, R^{M1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is phenyl or a 3-7 membered saturated or partially unsaturated monocyclic carbocyclic ring; each of which is substituted by z⁶ instances of R^{M1C}. In some embodiments, R^{M1A} is naphthyl or a 5-12 membered saturated or partially unsaturated bicyclic carbocyclic ring; each of which is substituted by z⁶ instances of R^{M1C}. In some embodiments, R^{M1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r3 instances of R^{M1C}. In some embodiments, R^{M1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}.

In some embodiments, R^{M1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁶ instances of R^{M1C}. In some embodiments, R^{M1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z⁶ instances of R^{M1C}. In some embodiments, R^{M1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁶ instances of R^{M1C}.

In some embodiments, R^{M1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z⁶ instances of R^{M1C}. In some embodiments, R^{M1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z⁶ instances of R^{M1C}. In some embodiments, R^{M1A} is a C₁₋₆ aliphatic chain, phenyl, or a 3-7 membered saturated or partially unsaturated monocyclic carbocyclic ring; each of which is substituted by z⁶ instances of R^{M1C}.

In some embodiments, R^{M1A} is selected from the groups depicted in the compounds in Table 1.

As defined generally above, R^{M2A} is R^{J} or R^{K} substituted by z⁷ instances of R^{M2C}. In some embodiments, R^{M2A} is R^{J}. In some embodiments, R^{M2A} is R^{K} substituted by z⁷ instances of R^{M2C}.

In some embodiments, R^{M2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{M2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M2A} is oxo. In some embodiments, R^{M2A} is halogen. In some embodiments, R^{M2A} is —CN. In some embodiments, R^{M2A} is —NO₂. In some embodiments, R^{M2A} is —OR. In some embodiments, R^{M2A} is —SR. In some embodiments, R^{M2A} is —NR₂. In some embodiments, R^{M2A} is —S(O)₂R. In some embodiments, R^{M2A} is —S(O)₂NR₂. In some embodiments, R^{M2A} is —S(O)₂F. In some embodiments, R^{M2A} is —S(O)R. In some embodiments, R^{M2A} is —S(O)NR₂. In some embodiments, R^{M2A} is —S(O)(NR)R. In some embodiments, R^{M2A} is —C(O)R. In some embodiments, R^{M2A} is —C(O)OR. In some embodiments, R^{M2A} is —C(O)NR₂. In some embodiments, R^{M2A} is —C(O)N(R)OR. In some embodiments, R^{M2A} is —OC(O)R. In some embodiments, R^{M2A} is —OC(O)NR₂. In some embodiments, R^{M2A} is —N(R)C(O)OR. In some embodiments, R^{M2A} is —N(R)C(O)R. In some embodiments, R^{M2A} is —N(R)C(O)NR₂. In some embodiments, R^{M2A} is —N(R)C(NR)NR₂. In some embodiments, R^{M2A} is —N(R)S(O)₂NR₂. In some embodiments, R^{M2A} is —N(R)S(O)₂R. In some embodiments, R^{M2A} is —P(O)R₂. In some embodiments, R^{M2A} is —P(O)(R)OR. In some embodiments, R^{M2A} is —B(OR)₂. In some embodiments, R^{M2A} is deuterium.

In some embodiments, R^{M2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{M2A} is halogen, —CN, or —NO₂. In some embodiments, R^{M2A} is —OR, —SR, or —NR₂. In some embodiments, R^{M2A} is —S(O)₂R, —S(O)₂NR₂, —S(O)₂F, —S(O)R, —S(O)NR₂, or —S(O)(NR)R. In some embodiments, R^{M2A} is —C(O)R, —C(O)OR, —C(O)NR₂, or —C(O)N(R)OR. In some embodiments, R^{M2A} is —OC(O)R or —OC(O)NR₂. In some embodiments, R^{M2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{M2A} is —P(O)R₂ or —P(O)(R)OR.

In some embodiments, R^{M2A} is —OR, —OC(O)R, or —OC(O)NR₂. In some embodiments, R^{M2A} is —SR, —S(O)₂R, —S(O)₂NR₂, —S(O)₂F, —S(O)R, —S(O)NR₂, or —S(O)(NR)R. In some embodiments, R^{M2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{M2A} is —S(O)₂R, —S(O)₂NR₂, or —S(O)₂F. In some embodiments, R^{M2A} is —S(O)R, —S(O)NR₂, or —S(O)(NR)R. In some embodiments, R^{M2A} is —SR, —S(O)₂R, or —S(O)R. In some embodiments, R^{M2A} is —S(O)₂NR₂, —S(O)NR₂, or —S(O)(NR)R. In some embodiments, R^{M2A} is —S(O)₂NR₂ or —S(O)NR₂. In some embodiments, R^{M2A} is —SR, —S(O)₂R, —S(O)₂NR₂, or —S(O)R.

In some embodiments, R^{M2A} is —N(R)C(O)OR, —N(R)C(O)R, or —N(R)C(O)NR₂. In some embodiments, R^{M2A} is —N(R)S(O)₂NR₂ or —N(R)S(O)₂R. In some embodiments, R^{M2A} is —N(R)C(O)OR or —N(R)C(O)R. In some embodiments, R^{M2A} is —N(R)C(O)NR₂ or —N(R)S(O)₂NR₂. In some embodiments, R^{M2A} is —N(R)C(O)OR, —N(R)C(O)R, or —N(R)S(O)₂R.

In some embodiments, R^{M2A} is —NR₂, —N(R)C(O)OR, —N(R)C(O)R, or —N(R)C(O)NR₂. In some embodiments, R^{M2A} is —NR₂, —N(R)C(O)OR, or —N(R)C(O)R. In some embodiments, R^{M2A} is —NR₂, —N(R)C(O)OR, —N(R)C(O)R, or —N(R)S(O)₂R.

In some embodiments, R^{M2A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is a C₁₋₆ aliphatic chain substituted by z⁷ instances of R^{M2C}. In some embodiments, R^{M2A} is phenyl substituted by z⁷ instances of R^{M2C}. In some embodiments, R^{M2A} is naphthyl substituted by z⁷ instances of R^{M2C}. In some embodiments, R^{M2A} is a 5-6 membered monocyclic heteroaryl ring having 1-4 heteroatoms independently selected from nitrogen, oxygen, and sulfur; wherein said ring is substituted by z⁷ instances of R^{M2C}. In some embodiments, R^{M2A} is an 8-10 membered bicyclic heteroaryl ring having 1-4 heteroatoms independently selected from nitrogen, oxygen, and sulfur; wherein said ring is substituted by z⁷ instances of R^{M2C}. In some embodiments, R^{M2A} is a 3-7 membered saturated or partially unsaturated monocyclic carbocyclic ring substituted by z⁷ instances of R^{M2C}. In some embodiments, R^{M2A} is a 5-12 membered saturated or partially unsaturated bicyclic carbocyclic ring substituted by z⁷ instances of R^{M2C}. In some embodiments, R^{M2A} is a 3-7 membered saturated or partially unsaturated monocyclic heterocyclic ring having 1-2 heteroatoms independently selected from nitrogen, oxygen, and sulfur; wherein said ring is substituted by z⁷ instances of R^{M2C}. In some embodiments, R^{M2A} is a 7-12 membered saturated or partially unsaturated bicyclic heterocyclic ring having 1-4 heteroatoms independently selected from nitrogen, oxygen, and sulfur; wherein said ring is substituted by z⁷ instances of R^{M2C}.

In some embodiments, R^{M2A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁷ instances of R^{M2C}. In some embodiments, R^{M2A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl; naphthyl; a 3-7 membered saturated or partially unsaturated monocyclic carbocyclic ring; or a 5-12 membered saturated or partially unsaturated bicyclic carbocyclic ring; each of which is substituted by z⁷ instances of R^{M2C}. In some embodiments, R^{M2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁷ instances of R^{M2C}. In some embodiments, R^{M2A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl or naphthyl; each of which is substituted by z⁷ instances of R^{M2C}. In some embodiments, R^{M2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁷ instances of R^{M2C}. In some embodiments, R^{M2A} is a 3-7 membered saturated or partially unsaturated monocyclic carbocyclic ring or a 5-12 membered saturated or partially unsaturated bicyclic carbocyclic ring; each of which is substituted by z⁷ instances of R^{M2C}. In some embodiments, R^{M2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl or a 5-6 membered monocyclic heteroaryl ring having 1-4 heteroatoms independently selected from nitrogen, oxygen, and sulfur; each of which is substituted by z⁷ instances of R^{M2C}. In some embodiments, R^{M2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z⁷ instances of R^{M2C}. In some embodiments, R^{M2A} is naphthyl or an 8-10 membered bicyclic heteroaryl ring having 1-4 heteroatoms independently selected from nitrogen, oxygen, and sulfur; each of which is substituted by z⁷ instances of R^{M2C}. In some embodiments, R^{M2A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is phenyl or a 3-7 membered saturated or partially unsaturated monocyclic carbocyclic ring; each of which is substituted by z⁷ instances of R^{M2C}. In some embodiments, R^{M2A} is naphthyl or a 5-12 membered saturated or partially unsaturated bicyclic carbocyclic ring; each of which is substituted by z⁷ instances of R^{M2C}. In some embodiments, R^{M2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by z⁷ instances of R^{M2C}. In some embodiments, R^{M2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}.

In some embodiments, R^{M2A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁷ instances of R^{M2C}. In some embodiments, R^{M2A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z⁷ instances of R^{M2C}. In some embodiments, R^{M2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁷ instances of R^{M2C}.

In some embodiments, R^{M2A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z⁷ instances of R^{M2C}. In some embodiments, R^{M2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z⁷ instances of R^{M2C}. In some embodiments, R^{M2A} is a C₁₋₆ aliphatic chain, phenyl, or a 3-7 membered saturated or partially unsaturated monocyclic carbocyclic ring; each of which is substituted by z⁷ instances of R^{M2C}.

In some embodiments, R^{M2A} is selected from the groups depicted in the compounds in Table 1.

As defined generally above, R^{L3} is R^{J} or R^{K} substituted by z⁸ instances of R^{L3C}. In some embodiments, R^{L3} is R^{J}. In some embodiments, R^{L3} is R^{K} substituted by z⁸ instances of R^{L3C}.

In some embodiments, R^{L3} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L3} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L3} is oxo. In some embodiments, R^{L3} is halogen. In some embodiments, R^{L3} is —CN. In some embodiments, R^{L3} is —NO₂. In some embodiments, R^{L3} is —OR. In some embodiments, R^{L3} is —SR. In some embodiments, R^{L3} is —NR₂. In some embodiments, R^{L3} is —S(O)₂R. In some embodiments, R^{L3} is —S(O)₂NR₂. In some embodiments, R^{L3} is —S(O)₂F. In some embodiments, R^{L3} is —S(O)R. In some embodiments, R^{L3} is —S(O)NR₂. In some embodiments, R^{L3} is —S(O)(NR)R. In some embodiments, R^{L3} is —C(O)R. In some embodiments, R^{L3} is —C(O)OR. In some embodiments, R^{L3} is —C(O)NR₂. In some embodiments, R^{L3} is —C(O)N(R)OR. In some embodiments, R^{L3} is —OC(O)R. In some embodiments, R^{L3} is —OC(O)NR₂. In some embodiments, R^{L3} is —N(R)C(O)OR. In some embodiments, R^{L3} is —N(R)C(O)R. In some embodiments, R^{L3} is —N(R)C(O)NR₂. In some embodiments, R^{L3} is —N(R)C(NR)NR₂. In some embodiments, R^{L3} is —N(R)S(O)₂NR₂. In some embodiments, R^{L3} is —N(R)S(O)₂R. In some embodiments, R^{L3} is —P(O)R₂. In some embodiments, R^{L3} is —P(O)(R)OR. In some embodiments, R^{L3} is —B(OR)₂. In some embodiments, R^{L3} is deuterium.

In some embodiments, R^{L3} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L3} is halogen, —CN, or —NO₂. In some embodiments, R^{L3} is —OR, —SR, or —NR₂. In some embodiments, R^{L3} is —S(O)₂R, —S(O)₂NR₂, —S(O)₂F, —S(O)R, —S(O)NR₂, or —S(O)(NR)R. In some embodiments, R^{L3} is —C(O)R, —C(O)OR, —C(O)NR₂, or —C(O)N(R)OR. In some embodiments, R^{L3} is —OC(O)R or —OC(O)NR₂. In some embodiments, R^{L3} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L3} is —P(O)R₂ or —P(O)(R)OR.

In some embodiments, R^{L3} is —OR, —OC(O)R, or —OC(O)NR₂. In some embodiments, R^{L3} is —SR, —S(O)₂R, —S(O)₂NR₂, —S(O)₂F, —S(O)R, —S(O)NR₂, or —S(O)(NR)R. In some embodiments, R^{L3} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L3} is —S(O)₂R, —S(O)₂NR₂, or —S(O)₂F. In some embodiments, R^{L3} is —S(O)R, —S(O)NR₂, or —S(O)(NR)R. In some embodiments, R^{L3} is —SR, —S(O)₂R, or —S(O)R. In some embodiments, R^{L3} is —S(O)₂NR₂, —S(O)NR₂, or —S(O)(NR)R. In some embodiments, R^{L3} is —S(O)₂NR₂ or —S(O)NR₂. In some embodiments, R^{L3} is —SR, —S(O)₂R, —S(O)₂NR₂, or —S(O)R.

In some embodiments, R^{L3} is —N(R)C(O)OR, —N(R)C(O)R, or —N(R)C(O)NR₂. In some embodiments, R^{L3} is —N(R)S(O)₂NR₂ or —N(R)S(O)₂R. In some embodiments, R^{L3} is —N(R)C(O)OR or —N(R)C(O)R. In some embodiments, R^{L3} is —N(R)C(O)NR₂ or —N(R)S(O)₂NR₂. In some embodiments, R^{L3} is —N(R)C(O)OR, —N(R)C(O)R, or —N(R)S(O)₂R.

In some embodiments, R^{L3} is —NR₂, —N(R)C(O)OR, —N(R)C(O)R, or —N(R)C(O)NR₂. In some embodiments, R^{L3} is —NR₂, —N(R)C(O)OR, or —N(R)C(O)R. In some embodiments, R^{L3} is —NR₂, —N(R)C(O)OR, —N(R)C(O)R, or —N(R)S(O)₂R.

In some embodiments, R^{L3} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is a C₁₋₆ aliphatic chain substituted by z⁸ instances of R^{L3C} In some embodiments, R^{L3} is phenyl substituted by z⁸ instances of R^{L3C}. In some embodiments, R^{L3} is naphthyl substituted by z⁸ instances of R^{L3C}. In some embodiments, R^{L3} is a 5-6 membered monocyclic heteroaryl ring having 1-4 heteroatoms independently selected from nitrogen, oxygen, and sulfur; wherein said ring is substituted by z⁸ instances of R^{L3C}. In some embodiments, R^{L3} is an 8-10 membered bicyclic heteroaryl ring having 1-4 heteroatoms independently selected from nitrogen, oxygen, and sulfur; wherein said ring is substituted by z⁸ instances of R^{L3C}. In some embodiments, R^{L3} is a 3-7 membered saturated or partially unsaturated monocyclic carbocyclic ring substituted by z⁸ instances of R^{L3C}. In some embodiments, R^{L3} is a 5-12 membered saturated or partially unsaturated bicyclic carbocyclic ring substituted by z⁸ instances of R^{L3C}. In some embodiments, R^{L3} is a 3-7 membered saturated or partially unsaturated monocyclic heterocyclic ring having 1-2 heteroatoms independently selected from nitrogen, oxygen, and sulfur; wherein said ring is substituted by z⁸ instances of R^{L3C}. In some embodiments, R^{L3} is a 7-12 membered saturated or partially unsaturated bicyclic heterocyclic ring having 1-4 heteroatoms independently selected from nitrogen, oxygen, and sulfur; wherein said ring is substituted by z⁸ instances of R^{L3C}.

In some embodiments, R^{L3} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁸ instances of R^{L3C}. In some embodiments, R^{L3} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl; naphthyl; a 3-7 membered saturated or partially unsaturated monocyclic carbocyclic ring; or a 5-12 membered saturated or partially unsaturated bicyclic carbocyclic ring; each of which is substituted by z⁸ instances of R^{L3C}. In some embodiments, R^{L3} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁸ instances of R^{L3C}. In some embodiments, R^{L3} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl or naphthyl; each of which is substituted by z⁸ instances of R^{L3C}. In some embodiments, R^{L3} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by z⁸ instances of R^{L3C}. In some embodiments, R^{L3} is a 3-7 membered saturated or partially unsaturated monocyclic carbocyclic ring or a 5-12 membered saturated or partially unsaturated bicyclic carbocyclic ring; each of which is substituted by z⁸ instances of R^{L3C}. In some embodiments, R^{L3} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl or a 5-6 membered monocyclic heteroaryl ring having 1-4 heteroatoms independently selected from nitrogen, oxygen, and sulfur; each of which is substituted by z⁸ instances of R^{L3C}. In some embodiments, R^{L3} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by z⁸ instances of R^{L3C}. In some embodiments, R¹³ is naphthyl or an 8-10 membered bicyclic heteroaryl ring having 1-4 heteroatoms independently selected from nitrogen, oxygen, and sulfur; each of which is substituted by z⁸ instances of R^{L3C}. In some embodiments, R^{L3} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is phenyl or a 3-7 membered saturated or partially unsaturated monocyclic carbocyclic ring; each of which is substituted by z⁸ instances of R^{L3C} In some embodiments, R^{L3} is naphthyl or a 5-12 membered saturated or partially unsaturated bicyclic carbocyclic ring; each of which is substituted by z⁸ instances of R^{L3C}. In some embodiments, R^{L3} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by z⁸ instances of R^{L3C}. In some embodiments, R^{L3} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}.

In some embodiments, R^{L3} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by z⁸ instances of R^{L3C}. In some embodiments, R^{L3} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by z⁸ instances of R^{L3C}. In some embodiments, R^{L3} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by z⁸ instances of R^{L3C}.

In some embodiments, R^{L3} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by z⁸ instances of R^{L3C}. In some embodiments, R^{L3} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by z⁸ instances of R^{L3C}. In some embodiments, R^{L3} is a C₁₋₆ aliphatic chain, phenyl, or a 3-7 membered saturated or partially unsaturated monocyclic carbocyclic ring; each of which is substituted by z⁸ instances of R^{L3C}.

In some embodiments, R^{L3} is selected from the groups depicted in the compounds in Table 1.

As defined generally above, Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur.

In some embodiments, Cy^{B} is phenyl. In some embodiments, Cy^{B} is a 5-6 membered monocyclic heteroaryl ring having 1-4 heteroatoms independently selected from nitrogen, oxygen, and sulfur. In some embodiments, Cy^{B} or an 8-10 membered bicyclic heteroaryl ring having 1-4 heteroatoms independently selected from nitrogen, oxygen, and sulfur. In some embodiments, Cy^{B} is selected from the groups depicted in the compounds in Table 1.

As defined generally above, R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur. In some embodiments, R^{CyB} is R^{J} or R^{K}. In some embodiments, R^{CyB} is R^{J}. In some embodiments, R^{CyB} is R^{K}. In some embodiments, R^{CyB} and R^{8C} are taken together with their intervening atoms to form a 5-6 membered heteroaryl ring having 1-4 heteroatoms independently selected from nitrogen, oxygen, and sulfur. In some embodiments, R^{CyB} and R^{8C} are taken together with their intervening atoms to form a 3-7 membered partially unsaturated heterocyclic ring having 1-2 heteroatoms independently selected from nitrogen, oxygen, and sulfur. In some embodiments, R^{CyB} is selected from the groups depicted in the compounds in Table 1.

As defined generally above, each instance of R is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{J} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{J} is oxo. In some embodiments, R^{J} is halogen. In some embodiments, R^{J} is —CN. In some embodiments, R^{J} is —NO₂. In some embodiments, R^{J} is —OR. In some embodiments, R^{J} is —SF₅. In some embodiments, R^{J} is —SR. In some embodiments, R^{J} is —NR₂. In some embodiments, R^{J} is —S(O)₂R. In some embodiments, R^{J} is —S(O)₂NR₂. In some embodiments, R^{J} is —S(O)₂F. In some embodiments, R^{J} is —S(O)R. In some embodiments, R^{J} is —S(O)NR₂. In some embodiments, R^{J} is —S(O)(NR)R. In some embodiments, R^{J} is —C(O)R. In some embodiments, R^{J} is —C(O)OR. In some embodiments, R^{J} is —C(O)NR₂. In some embodiments, R^{J} is —C(O)N(R)OR. In some embodiments, R^{J} is —OC(O)R. In some embodiments, R^{J} is —OC(O)NR₂. In some embodiments, R^{J} is —N(R)C(O)OR. In some embodiments, R^{J} is —N(R)C(O)R. In some embodiments, R^{J} is —N(R)C(O)NR₂. In some embodiments, R^{J} is —N(R)C(NR)NR₂. In some embodiments, R^{J} is —N(R)S(O)₂NR₂. In some embodiments, R^{J} is —N(R)S(O)₂R. In some embodiments, R^{J} is —P(O)R₂. In some embodiments, R^{J} is —P(O)(R)OR. In some embodiments, R^{J} is —B(OR)₂. In some embodiments, R^{J} is deuterium.

In some embodiments, R^{J} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{J} is halogen, —CN, or —NO₂. In some embodiments, R^{J} is —OR, —SR, or —NR₂. In some embodiments, R^{J} is —S(O)₂R, —S(O)₂NR₂, —S(O)₂F, —S(O)R, —S(O)NR₂, or —S(O)(NR)R. In some embodiments, R^{J} is —C(O)R, —C(O)OR, —C(O)NR₂, or —C(O)N(R)OR. In some embodiments, R^{J} is —OC(O)R or —OC(O)NR₂. In some embodiments, R^{J} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{J} is —P(O)R₂ or —P(O)(R)OR.

In some embodiments, R^{J} is —OR, —OC(O)R, or —OC(O)NR₂. In some embodiments, R^{J} is —SR, —S(O)₂R, —S(O)₂NR₂, —S(O)₂F, —S(O)R, —S(O)NR₂, or —S(O)(NR)R. In some embodiments, R^{J} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{J} is —S(O)₂R, —S(O)₂NR₂, or —S(O)₂F. In some embodiments, R^{J} is —S(O)R, —S(O)NR₂, or —S(O)(NR)R. In some embodiments, R^{J} is —SR, —S(O)₂R, or —S(O)R. In some embodiments, R^{J} is —S(O)₂NR₂, —S(O)NR₂, or —S(O)(NR)R. In some embodiments, R^{J} is —S(O)₂NR₂ or —S(O)NR₂. In some embodiments, R^{J} is —SR, —S(O)₂R, —S(O)₂NR₂, or —S(O)R.

In some embodiments, R^{J} is —N(R)C(O)OR, —N(R)C(O)R, or —N(R)C(O)NR₂. In some embodiments, R^{J} is —N(R)S(O)₂NR₂ or —N(R)S(O)₂R. In some embodiments, R^{J} is —N(R)C(O)OR or —N(R)C(O)R. In some embodiments, R^{J} is —N(R)C(O)NR₂ or —N(R)S(O)₂NR₂. In some embodiments, R^{J} is —N(R)C(O)OR, —N(R)C(O)R, or —N(R)S(O)₂R.

In some embodiments, R^{J} is —NR₂, —N(R)C(O)OR, —N(R)C(O)R, or —N(R)C(O)NR₂. In some embodiments, R^{J} is —NR₂, —N(R)C(O)OR, or —N(R)C(O)R. In some embodiments, R^{J} is —NR₂, —N(R)C(O)OR, —N(R)C(O)R, or —N(R)S(O)₂R.

In some embodiments, R^{J} is selected from the groups depicted in the compounds in Table 1.

As defined generally above, each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is a C₁₋₆ aliphatic chain. In some embodiments, R^{K} is phenyl. In some embodiments, R^{K} is naphthyl. In some embodiments, R^{K} is cubanyl. In some embodiments, R^{K} is adamantyl. In some embodiments, R^{K} is a 5-6 membered monocyclic heteroaryl ring having 1-4 heteroatoms independently selected from nitrogen, oxygen, and sulfur. In some embodiments, R^{K} is an 8-10 membered bicyclic heteroaryl ring having 1-4 heteroatoms independently selected from nitrogen, oxygen, and sulfur. In some embodiments, R^{K} is a 3-7 membered saturated or partially unsaturated monocyclic carbocyclic ring. In some embodiments, R^{K} is a 5-12 membered saturated or partially unsaturated bicyclic carbocyclic ring. In some embodiments, R^{K} is a 3-7 membered saturated or partially unsaturated monocyclic heterocyclic ring having 1-2 heteroatoms independently selected from nitrogen, oxygen, and sulfur. In some embodiments, R^{K} is a 7-12 membered saturated or partially unsaturated bicyclic heterocyclic ring having 1-4 heteroatoms independently selected from nitrogen, oxygen, and sulfur.

In some embodiments, R^{K} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{K} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{K} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{K} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{K} is phenyl or naphthyl. In some embodiments, R^{K} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{K} is a 3-7 membered saturated or partially unsaturated monocyclic carbocyclic ring or a 5-12 membered saturated or partially unsaturated bicyclic carbocyclic ring. In some embodiments, R^{K} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is phenyl or a 5-6 membered monocyclic heteroaryl ring having 1-4 heteroatoms independently selected from nitrogen, oxygen, and sulfur. In some embodiments, R^{K} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{K} is naphthyl or an 8-10 membered bicyclic heteroaryl ring having 1-4 heteroatoms independently selected from nitrogen, oxygen, and sulfur. In some embodiments, R^{K} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{K} is phenyl or a 3-7 membered saturated or partially unsaturated monocyclic carbocyclic ring. In some embodiments, R^{K} is naphthyl or a 5-12 membered saturated or partially unsaturated bicyclic carbocyclic ring. In some embodiments, R^{K} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{K} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{K} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{K} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{K} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{K} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{K} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{K} is a C₁₋₆ aliphatic chain, phenyl, or a 3-7 membered saturated or partially unsaturated monocyclic carbocyclic ring.

In some embodiments, R^{K} is selected from the groups depicted in the compounds in Table 1.

As defined generally above, each instance of R^{7C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{7C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{7C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{7C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{7C} is oxo. In some embodiments, R^{7C} is deuterium. In some embodiments, each instance of R^{7C} is independently halogen. In some embodiments, R^{7C} is —CN. In some embodiments, R^{7C} is —NO₂. In some embodiments, R^{7C} is —OR. In some embodiments, R^{7C} is —SR. In some embodiments, R^{7C} is —NR₂. In some embodiments, R^{7C} is —S(O)₂R. In some embodiments, R^{7C} is —S(O)₂NR₂. In some embodiments, R^{7C} is —S(O)₂F. In some embodiments, R^{7C} is —S(O)R. In some embodiments, R^{7C} is —S(O)NR₂. In some embodiments, R^{7C} is —S(O)(NR)R. In some embodiments, R^{7C} is —C(O)R. In some embodiments, R^{7C} is —C(O)OR. In some embodiments, R^{7C} is —C(O)NR₂. In some embodiments, R^{7C} is —C(O)N(R)OR. In some embodiments, R^{7C} is —OC(O)R. In some embodiments, R^{7C} is —OC(O)NR₂. In some embodiments, R^{7C} is —N(R)C(O)OR. In some embodiments, R^{7C} is —N(R)C(O)R. In some embodiments, R^{7C} is —N(R)C(O)NR₂. In some embodiments, R^{7C} is —N(R)C(NR)NR₂. In some embodiments, R^{7C} is —N(R)S(O)₂NR₂. In some embodiments, R^{7C} is —N(R)S(O)₂R. In some embodiments, R^{7C} is —P(O)R₂. In some embodiments, R^{7C} is —P(O)(R)OR. In some embodiments, R^{7C} is —B(OR)₂.

In some embodiments, each instance of R^{7C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{7C} is independently halogen, —CN, or —NO₂. In some embodiments, each instance of R^{7C} is independently —OR, —SR, or —NR₂. In some embodiments, each instance of R^{7C} is independently —S(O)₂R, —S(O)₂NR₂, —S(O)₂F, —S(O)R, —S(O)NR₂, or —S(O)(NR)R. In some embodiments, each instance of R^{7C} is independently —C(O)R, —C(O)OR, —C(O)NR₂, or —C(O)N(R)OR. In some embodiments, each instance of R^{7C} is independently —OC(O)R or —OC(O)NR₂. In some embodiments, each instance of R^{7C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{7C} is independently —P(O)R₂ or —P(O)(R)OR.

In some embodiments, each instance of R^{7C} is independently —OR, —OC(O)R, or —OC(O)NR₂. In some embodiments, each instance of R^{7C} is independently —SR, —S(O)₂R, —S(O)₂NR₂, —S(O)₂F, —S(O)R, —S(O)NR₂, or —S(O)(NR)R. In some embodiments, each instance of R^{7C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{7C} is independently —S(O)₂R, —S(O)₂NR₂, or —S(O)₂F. In some embodiments, each instance of R^{7C} is independently —S(O)R, —S(O)NR₂, or —S(O)(NR)R. In some embodiments, each instance of R^{7C} is independently —SR, —S(O)₂R, or —S(O)R. In some embodiments, each instance of R^{7C} is independently —S(O)₂NR₂, —S(O)NR₂, or —S(O)(NR)R. In some embodiments, each instance of R^{7C} is independently —S(O)₂NR₂ or —S(O)NR₂. In some embodiments, each instance of R^{7C} is independently —SR, —S(O)₂R, —S(O)₂NR₂, or —S(O)R.

In some embodiments, each instance of R^{7C} is independently —N(R)C(O)OR, —N(R)C(O)R, or —N(R)C(O)NR₂. In some embodiments, each instance of R^{7C} is independently —N(R)S(O)₂NR₂ or —N(R)S(O)₂R. In some embodiments, each instance of R^{7C} is independently —N(R)C(O)OR or —N(R)C(O)R. In some embodiments, each instance of R^{7C} is independently —N(R)C(O)NR₂ or —N(R)S(O)₂NR₂. In some embodiments, each instance of R^{7C} is independently —N(R)C(O)OR, —N(R)C(O)R, or —N(R)S(O)₂R.

In some embodiments, each instance of R^{7C} is independently —NR₂, —N(R)C(O)OR, —N(R)C(O)R, or —N(R)C(O)NR₂. In some embodiments, each instance of R^{7C} is independently —NR₂, —N(R)C(O)OR, or —N(R)C(O)R. In some embodiments, each instance of R^{7C} is independently —NR₂, —N(R)C(O)OR, —N(R)C(O)R, or —N(R)S(O)₂R.

In some embodiments, each instance of R^{7C} is independently an optionally substituted C₁₋₆ aliphatic. In some embodiments, each instance of R^{7C} is independently an optionally substituted phenyl. In some embodiments, each instance of R^{7C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{7C} is independently an optionally substituted 5-6 membered monocyclic heteroaryl ring having 1-4 heteroatoms independently selected from nitrogen, oxygen, and sulfur.

In some embodiments, each instance of R^{7C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{7C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{7C} is independently an optionally substituted C₁₋₆ aliphatic or an optionally substituted phenyl. In some embodiments, each instance of R^{7C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{7C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{7C} is independently a C₁₋₆ aliphatic. In some embodiments, R^{7C} is phenyl. In some embodiments, each instance of R^{7C} is independently a 3-7 membered saturated or partially unsaturated monocyclic heterocyclic ring having 1-2 heteroatoms independently selected from nitrogen, oxygen, and sulfur. In some embodiments, each instance of R^{7C} is independently a 5-6 membered monocyclic heteroaryl ring having 1-4 heteroatoms independently selected from nitrogen, oxygen, and sulfur.

In some embodiments, each instance of R^{7C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{7C} is independently phenyl or a 5-6 membered monocyclic heteroaryl ring having 1-4 heteroatoms independently selected from nitrogen, oxygen, and sulfur.

In some embodiments, each instance of R^{7C} is independently a C₁₋₆ aliphatic or phenyl. In some embodiments, each instance of R^{7C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{7C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{7C} is independently halogen, —CN, —O-(optionally substituted C₁₋₆ aliphatic), or an optionally substituted C₁₋₆ aliphatic. In some embodiments, each instance of R^{7C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{7C} is independently halogen or C₁₋₃ aliphatic optionally substituted with 1-3 halogen. In some embodiments, each instance of R^{7C} is independently fluorine, chlorine, —CH₃, —CHF₂, or —CF₃.

In some embodiments, each instance of R^{7C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{7C} is independently selected from the groups depicted in the compounds in Table 1.

As defined generally above, each instance of R^{8C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{8C} is independently —OH, —C(O)OH, —C(O)NH, —S(O)NH₂, or —S(O)₂NH₂.

In some embodiments, each instance of R^{8C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{8C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{8C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{8C} is oxo. In some embodiments, R^{8C} is deuterium. In some embodiments, each instance of R^{8C} is independently halogen. In some embodiments, R^{8C} is —CN. In some embodiments, R^{8C} is —NO₂. In some embodiments, R^{8C} is —OR. In some embodiments, R^{8C} is —SR. In some embodiments, R^{8C} is —NR₂. In some embodiments, R^{8C} is —S(O)₂R. In some embodiments, R^{8C} is —S(O)₂NR₂. In some embodiments, R^{8C} is —S(O)₂F. In some embodiments, R^{8C} is —S(O)R. In some embodiments, R^{8C} is —S(O)NR₂. In some embodiments, R^{8C} is —S(O)(NR)R. In some embodiments, R^{8C} is —C(O)R. In some embodiments, R^{8C} is —C(O)OR. In some embodiments, R^{8C} is —C(O)NR₂. In some embodiments, R^{8C} is —C(O)N(R)OR. In some embodiments, R^{8C} is —OC(O)R. In some embodiments, R^{8C} is —OC(O)NR₂. In some embodiments, R^{8C} is —N(R)C(O)OR. In some embodiments, R^{8C} is —N(R)C(O)R. In some embodiments, R^{8C} is —N(R)C(O)NR₂. In some embodiments, R^{8C} is —N(R)C(NR)NR₂. In some embodiments, R^{8C} is —N(R)S(O)₂NR₂. In some embodiments, R^{8C} is —N(R)S(O)₂R. In some embodiments, R^{8C} is —P(O)R₂. In some embodiments, R^{8C} is —P(O)(R)OR. In some embodiments, R^{8C} is —B(OR)₂.

In some embodiments, each instance of R^{8C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{8C} is independently halogen, —CN, or —NO₂. In some embodiments, each instance of R^{8C} is independently —OR, —SR, or —NR₂. In some embodiments, each instance of R^{8C} is independently —S(O)₂R, —S(O)₂NR₂, —S(O)₂F, —S(O)R, —S(O)NR₂, or —S(O)(NR)R. In some embodiments, each instance of R^{8C} is independently —C(O)R, —C(O)OR, —C(O)NR₂, or —C(O)N(R)OR. In some embodiments, each instance of R^{8C} is independently —OC(O)R or —OC(O)NR₂. In some embodiments, each instance of R^{8C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{8C} is independently —P(O)R₂ or —P(O)(R)OR.

In some embodiments, each instance of R^{8C} is independently —OR, —OC(O)R, or —OC(O)NR₂. In some embodiments, each instance of R^{8C} is independently —SR, —S(O)₂R, —S(O)₂NR₂, —S(O)₂F, —S(O)R, —S(O)NR₂, or —S(O)(NR)R. In some embodiments, each instance of R^{8C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{8C} is independently —S(O)₂R, —S(O)₂NR₂, or —S(O)₂F. In some embodiments, each instance of R^{8C} is independently —S(O)R, —S(O)NR₂, or —S(O)(NR)R. In some embodiments, each instance of R^{8C} is independently —SR, —S(O)₂R, or —S(O)R. In some embodiments, each instance of R^{8C} is independently —S(O)₂NR₂, —S(O)NR₂, or —S(O)(NR)R. In some embodiments, each instance of R^{8C} is independently —S(O)₂NR₂ or —S(O)NR₂. In some embodiments, each instance of R^{8C} is independently —SR, —S(O)₂R, —S(O)₂NR₂, or —S(O)R.

In some embodiments, each instance of R^{8C} is independently —N(R)C(O)OR, —N(R)C(O)R, or —N(R)C(O)NR₂. In some embodiments, each instance of R^{8C} is independently —N(R)S(O)₂NR₂ or —N(R)S(O)₂R. In some embodiments, each instance of R^{8C} is independently —N(R)C(O)OR or —N(R)C(O)R. In some embodiments, each instance of R^{8C} is independently —N(R)C(O)NR₂ or —N(R)S(O)₂NR₂. In some embodiments, each instance of R^{8C} is independently —N(R)C(O)OR, —N(R)C(O)R, or —N(R)S(O)₂R.

In some embodiments, each instance of R^{8C} is independently —NR₂, —N(R)C(O)OR, —N(R)C(O)R, or —N(R)C(O)NR₂. In some embodiments, each instance of R^{8C} is independently —NR₂, —N(R)C(O)OR, or —N(R)C(O)R. In some embodiments, each instance of R^{8C} is independently —NR₂, —N(R)C(O)OR, —N(R)C(O)R, or —N(R)S(O)₂R.

In some embodiments, each instance of R^{8C} is independently an optionally substituted C₁₋₆ aliphatic. In some embodiments, each instance of R^{8C} is independently an optionally substituted phenyl. In some embodiments, each instance of R^{8C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{8C} is independently an optionally substituted 5-6 membered monocyclic heteroaryl ring having 1-4 heteroatoms independently selected from nitrogen, oxygen, and sulfur.

In some embodiments, each instance of R^{8C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{8C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{8C} is independently an optionally substituted C₁₋₆ aliphatic or an optionally substituted phenyl. In some embodiments, each instance of R^{8C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{8C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{8C} is independently a C₁₋₆ aliphatic. In some embodiments, R^{8C} is phenyl. In some embodiments, each instance of R^{8C} is independently a 3-7 membered saturated or partially unsaturated monocyclic heterocyclic ring having 1-2 heteroatoms independently selected from nitrogen, oxygen, and sulfur. In some embodiments, each instance of R^{8C} is independently a 5-6 membered monocyclic heteroaryl ring having 1-4 heteroatoms independently selected from nitrogen, oxygen, and sulfur.

In some embodiments, each instance of R^{8C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{8C} is independently phenyl or a 5-6 membered monocyclic heteroaryl ring having 1-4 heteroatoms independently selected from nitrogen, oxygen, and sulfur.

In some embodiments, each instance of R^{8C} is independently a C₁₋₆ aliphatic or phenyl. In some embodiments, each instance of R^{8C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{8C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and

In some embodiments, each instance of R^{8C} is independently halogen, —CN, —O-(optionally substituted C₁₋₆ aliphatic), or an optionally substituted C₁₋₆ aliphatic. In some embodiments, each instance of R^{8C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{8C} is independently halogen or C₁₋₃ aliphatic optionally substituted with 1-3 halogen. In some embodiments, each instance of R^{8C} is independently fluorine, chlorine, —CH₃, —CHF₂, or —CF₃.

In some embodiments, each instance of R^{8C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{8C} is independently selected from the groups depicted in the compounds in Table 1.

As defined generally above, each instance of R^{G1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{G1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{G1C} is oxo. In some embodiments, R^{G1C} is deuterium. In some embodiments, each instance of R^{G1C} is independently halogen. In some embodiments, R^{G1C} is —CN. In some embodiments, R^{G1C} is —NO₂. In some embodiments, R^{G1C} is —OR. In some embodiments, R^{G1C} is —SR. In some embodiments, R^{G1C} is —NR₂. In some embodiments, R^{G1C} is —S(O)₂R. In some embodiments, R^{G1C} is —S(O)₂NR₂. In some embodiments, R^{G1C} is —S(O)₂F. In some embodiments, R^{G1C} is —S(O)R. In some embodiments, R^{G1C} is —S(O)NR₂. In some embodiments, R^{G1C} is —S(O)(NR)R. In some embodiments, R^{G1C} is —C(O)R. In some embodiments, R^{G1C} is —C(O)OR. In some embodiments, R^{G1C} is —C(O)NR₂. In some embodiments, R^{G1C} is —C(O)N(R)OR. In some embodiments, R^{G1C} is —OC(O)R. In some embodiments, R^{G1C} is —OC(O)NR₂. In some embodiments, R^{G1C} is —N(R)C(O)OR. In some embodiments, R^{G1C} is —N(R)C(O)R. In some embodiments, R^{G1C} is —N(R)C(O)NR₂. In some embodiments, R^{G1C} is —N(R)C(NR)NR₂. In some embodiments, R^{G1C} is —N(R)S(O)₂NR₂. In some embodiments, R^{G1C} is —N(R)S(O)₂R. In some embodiments, R^{G1C} is —P(O)R₂. In some embodiments, R^{G1C} is —P(O)(R)OR. In some embodiments, R^{G1C} is —B(OR)₂.

In some embodiments, each instance of R^{G1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{G1C} is independently halogen, —CN, or —NO₂. In some embodiments, each instance of R^{G1C} is independently —OR, —SR, or —NR₂. In some embodiments, each instance of R^{G1C} is independently —S(O)₂R, —S(O)₂NR₂, —S(O)₂F, —S(O)R, —S(O)NR₂, or —S(O)(NR)R. In some embodiments, each instance of R^{G1C} is independently —C(O)R, —C(O)OR, —C(O)NR₂, or —C(O)N(R)OR. In some embodiments, each instance of R^{G1C} is independently —OC(O)R or —OC(O)NR₂. In some embodiments, each instance of R^{G1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{G1C} is independently —P(O)R₂ or —P(O)(R)OR.

In some embodiments, each instance of R^{G1C} is independently —OR, —OC(O)R, or —OC(O)NR₂. In some embodiments, each instance of R^{G1C} is independently —SR, —S(O)₂R, —S(O)₂NR₂, —S(O)₂F, —S(O)R, —S(O)NR₂, or —S(O)(NR)R. In some embodiments, each instance of R^{G1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{G1C} is independently —S(O)₂R, —S(O)₂NR₂, or —S(O)₂F. In some embodiments, each instance of R^{G1C} is independently —S(O)R, —S(O)NR₂, or —S(O)(NR)R. In some embodiments, each instance of R^{G1C} is independently —SR, —S(O)₂R, or —S(O)R. In some embodiments, each instance of R^{G1C} is independently —S(O)₂NR₂, —S(O)NR₂, or —S(O)(NR)R. In some embodiments, each instance of R^{G1C} is independently —S(O)₂NR₂ or —S(O)NR₂. In some embodiments, each instance of R^{G1C} is independently —SR, —S(O)₂R, —S(O)₂NR₂, or —S(O)R.

In some embodiments, each instance of R^{G1C} is independently —N(R)C(O)OR, —N(R)C(O)R, or —N(R)C(O)NR₂. In some embodiments, each instance of R^{G1C} is independently —N(R)S(O)₂NR₂ or —N(R)S(O)₂R. In some embodiments, each instance of R^{G1C} is independently —N(R)C(O)OR or —N(R)C(O)R. In some embodiments, each instance of R^{G1C} is independently —N(R)C(O)NR₂ or —N(R)S(O)₂NR₂. In some embodiments, each instance of R^{G1C} is independently —N(R)C(O)OR, —N(R)C(O)R, or —N(R)S(O)₂R.

In some embodiments, each instance of R^{G1C} is independently —NR₂, —N(R)C(O)OR, —N(R)C(O)R, or —N(R)C(O)NR₂. In some embodiments, each instance of R^{G1C} is independently —NR₂, —N(R)C(O)OR, or —N(R)C(O)R. In some embodiments, each instance of R^{G1C} is independently —NR₂, —N(R)C(O)OR, —N(R)C(O)R, or —N(R)S(O)₂R.

In some embodiments, each instance of R^{G1C} is independently an optionally substituted C₁₋₆ aliphatic. In some embodiments, each instance of R^{G1C} is independently an optionally substituted phenyl. In some embodiments, each instance of R^{G1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{G1C} is independently an optionally substituted 5-6 membered monocyclic heteroaryl ring having 1-4 heteroatoms independently selected from nitrogen, oxygen, and sulfur.

In some embodiments, each instance of R^{G1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{G1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{G1C} is independently an optionally substituted C₁₋₆ aliphatic or an optionally substituted phenyl. In some embodiments, each instance of R^{G1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{G1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G1C} is independently a C₁₋₆ aliphatic. In some embodiments, R^{G1C} is phenyl. In some embodiments, each instance of R^{G1C} is independently a 3-7 membered saturated or partially unsaturated monocyclic heterocyclic ring having 1-2 heteroatoms independently selected from nitrogen, oxygen, and sulfur. In some embodiments, each instance of R^{G1C} is independently a 5-6 membered monocyclic heteroaryl ring having 1-4 heteroatoms independently selected from nitrogen, oxygen, and

In some embodiments, each instance of R^{G1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{G1C} is independently phenyl or a 5-6 membered monocyclic heteroaryl ring having 1-4 heteroatoms independently selected from nitrogen, oxygen, and sulfur.

In some embodiments, each instance of R^{G1C} is independently a C₁₋₆ aliphatic or phenyl. In some embodiments, each instance of R^{G1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G1C} is independently selected from the groups depicted in the compounds in Table 1.

As defined generally above, each instance of R^{G2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{G2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{G2C} is oxo. In some embodiments, R^{G2C} is deuterium. In some embodiments, each instance of R^{G2C} is independently halogen. In some embodiments, R^{G2C} is —CN. In some embodiments, R^{G2C} is —NO₂. In some embodiments, R^{G2C} is —OR. In some embodiments, R^{G2C} is —SR. In some embodiments, R^{G2C} is —NR₂. In some embodiments, R^{G2C} is —S(O)₂R. In some embodiments, R^{G2C} is —S(O)₂NR₂. In some embodiments, R^{G2C} is —S(O)₂F. In some embodiments, R^{G2C} is —S(O)R. In some embodiments, R^{G2C} is —S(O)NR₂. In some embodiments, R^{G2C} is —S(O)(NR)R. In some embodiments, R^{G2C} is —C(O)R. In some embodiments, R^{G2C} is —C(O)OR. In some embodiments, R^{G2C} is —C(O)NR₂. In some embodiments, R^{G2C} is —C(O)N(R)OR. In some embodiments, R^{G2C} is —OC(O)R. In some embodiments, R^{G2C} is —OC(O)NR₂. In some embodiments, R^{G2C} is —N(R)C(O)OR. In some embodiments, R^{G2C} is —N(R)C(O)R. In some embodiments, R^{G2C} is —N(R)C(O)NR₂. In some embodiments, R^{G2C} is —N(R)C(NR)NR₂. In some embodiments, R^{G2C} is —N(R)S(O)₂NR₂. In some embodiments, R^{G2C} is —N(R)S(O)₂R. In some embodiments, R^{G2C} is —P(O)R₂. In some embodiments, R^{G2C} is —P(O)(R)OR. In some embodiments, R^{G2C} is —B(OR)₂.

In some embodiments, each instance of R^{G2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{G2C} is independently halogen, —CN, or —NO₂. In some embodiments, each instance of R^{G2C} is independently —OR, —SR, or —NR₂. In some embodiments, each instance of R^{G2C} is independently —S(O)₂R, —S(O)₂NR₂, —S(O)₂F, —S(O)R, —S(O)NR₂, or —S(O)(NR)R. In some embodiments, each instance of R^{G2C} is independently —C(O)R, —C(O)OR, —C(O)NR₂, or —C(O)N(R)OR. In some embodiments, each instance of R^{G2C} is independently —OC(O)R or —OC(O)NR₂. In some embodiments, each instance of R^{G2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{G2C} is independently —P(O)R₂ or —P(O)(R)OR.

In some embodiments, each instance of R^{G2C} is independently —OR, —OC(O)R, or —OC(O)NR₂. In some embodiments, each instance of R^{G2C} is independently —SR, —S(O)₂R, —S(O)₂NR₂, —S(O)₂F, —S(O)R, —S(O)NR₂, or —S(O)(NR)R. In some embodiments, each instance of R^{G2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{G2C} is independently —S(O)₂R, —S(O)₂NR₂, or —S(O)₂F. In some embodiments, each instance of R^{G2C} is independently —S(O)R, —S(O)NR₂, or —S(O)(NR)R. In some embodiments, each instance of R^{G2C} is independently —SR, —S(O)₂R, or —S(O)R. In some embodiments, each instance of R^{G2C} is independently —S(O)₂NR₂, —S(O)NR₂, or —S(O)(NR)R. In some embodiments, each instance of R^{G2C} is independently —S(O)₂NR₂ or —S(O)NR₂. In some embodiments, each instance of R^{G2C} is independently —SR, —S(O)₂R, —S(O)₂NR₂, or —S(O)R.

In some embodiments, each instance of R^{G2C} is independently —N(R)C(O)OR, —N(R)C(O)R, or —N(R)C(O)NR₂. In some embodiments, each instance of R^{G2C} is independently —N(R)S(O)₂NR₂ or —N(R)S(O)₂R. In some embodiments, each instance of R^{G2C} is independently —N(R)C(O)OR or —N(R)C(O)R. In some embodiments, each instance of R^{G2C} is independently —N(R)C(O)NR₂ or —N(R)S(O)₂NR₂. In some embodiments, each instance of R^{G2C} is independently —N(R)C(O)OR, —N(R)C(O)R, or —N(R)S(O)₂R.

In some embodiments, each instance of R^{G2C} is independently —NR₂, —N(R)C(O)OR, —N(R)C(O)R, or —N(R)C(O)NR₂. In some embodiments, each instance of R^{G2C} is independently —NR₂, —N(R)C(O)OR, or —N(R)C(O)R. In some embodiments, each instance of R^{G2C} is independently —NR₂, —N(R)C(O)OR, —N(R)C(O)R, or —N(R)S(O)₂R.

In some embodiments, each instance of R^{G2C} is independently an optionally substituted C₁₋₆ aliphatic. In some embodiments, each instance of R^{G2C} is independently an optionally substituted phenyl. In some embodiments, each instance of R^{G2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{G2C} is independently an optionally substituted 5-6 membered monocyclic heteroaryl ring having 1-4 heteroatoms independently selected from nitrogen, oxygen, and sulfur.

In some embodiments, each instance of R^{G2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{G2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{G2C} is independently an optionally substituted C₁₋₆ aliphatic or an optionally substituted phenyl. In some embodiments, each instance of R^{G2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{G2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G2C} is independently a C₁₋₆ aliphatic. In some embodiments, R^{G2C} is phenyl. In some embodiments, each instance of R^{G2C} is independently a 3-7 membered saturated or partially unsaturated monocyclic heterocyclic ring having 1-2 heteroatoms independently selected from nitrogen, oxygen, and sulfur. In some embodiments, each instance of R^{G2C} is independently a 5-6 membered monocyclic heteroaryl ring having 1-4 heteroatoms independently selected from nitrogen, oxygen, and sulfur.

In some embodiments, each instance of R^{G2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{G2C} is independently phenyl or a 5-6 membered monocyclic heteroaryl ring having 1-4 heteroatoms independently selected from nitrogen, oxygen, and sulfur.

In some embodiments, each instance of R^{G2C} is independently a C₁₋₆ aliphatic or phenyl. In some embodiments, each instance of R^{G2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G2C} is independently selected from the groups depicted in the compounds in Table 1.

As defined generally above, each instance of R^{G3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{G3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{G3C} is oxo. In some embodiments, R^{G3C} is deuterium. In some embodiments, each instance of R^{G3C} is independently halogen. In some embodiments, R^{G3C} is —CN. In some embodiments, R^{G3C} is —NO₂. In some embodiments, R^{G3C} is —OR. In some embodiments, R^{G3C} is —SR. In some embodiments, R^{G3C} is —NR₂. In some embodiments, R^{G3C} is —S(O)₂R. In some embodiments, R^{G3C} is —S(O)₂NR₂. In some embodiments, R^{G3C} is —S(O)₂F. In some embodiments, R^{G3C} is —S(O)R. In some embodiments, R^{G3C} is —S(O)NR₂. In some embodiments, R^{G3C} is —S(O)(NR)R. In some embodiments, R^{G3C} is —C(O)R. In some embodiments, R^{G3C} is —C(O)OR. In some embodiments, R^{G3C} is —C(O)NR₂. In some embodiments, R^{G3C} is —C(O)N(R)OR. In some embodiments, R^{G3C} is —OC(O)R. In some embodiments, R^{G3C} is —OC(O)NR₂. In some embodiments, R^{G3C} is —N(R)C(O)OR. In some embodiments, R^{G3C} is —N(R)C(O)R. In some embodiments, R^{G3C} is —N(R)C(O)NR₂. In some embodiments, R^{G3C} is —N(R)C(NR)NR₂. In some embodiments, R^{G3C} is —N(R)S(O)₂NR₂. In some embodiments, R^{G3C} is —N(R)S(O)₂R. In some embodiments, R^{G3C} is —P(O)R₂. In some embodiments, R^{G3C} is —P(O)(R)OR. In some embodiments, R^{G3C} is —B(OR)₂.

In some embodiments, each instance of R^{G3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{G3C} is independently halogen, —CN, or —NO₂. In some embodiments, each instance of R^{G3C} is independently —OR, —SR, or —NR₂. In some embodiments, each instance of R^{G3C} is independently —S(O)₂R, —S(O)₂NR₂, —S(O)₂F, —S(O)R, —S(O)NR₂, or —S(O)(NR)R. In some embodiments, each instance of R^{G3C} is independently —C(O)R, —C(O)OR, —C(O)NR₂, or —C(O)N(R)OR. In some embodiments, each instance of R^{G3C} is independently —OC(O)R or —OC(O)NR₂. In some embodiments, each instance of R^{G3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{G3C} is independently —P(O)R₂ or —P(O)(R)OR.

In some embodiments, each instance of R^{G3C} is independently —OR, —OC(O)R, or —OC(O)NR₂. In some embodiments, each instance of R^{G3C} is independently —SR, —S(O)₂R, —S(O)₂NR₂, —S(O)₂F, —S(O)R, —S(O)NR₂, or —S(O)(NR)R. In some embodiments, each instance of R^{G3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{G3C} is independently —S(O)₂R, —S(O)₂NR₂, or —S(O)₂F. In some embodiments, each instance of R^{G3C} is independently —S(O)R, —S(O)NR₂, or —S(O)(NR)R. In some embodiments, each instance of R^{G3C} is independently —SR, —S(O)₂R, or —S(O)R. In some embodiments, each instance of R^{G3C} is independently —S(O)₂NR₂, —S(O)NR₂, or —S(O)(NR)R. In some embodiments, each instance of R^{G3C} is independently —S(O)₂NR₂ or —S(O)NR₂. In some embodiments, each instance of R^{G3C} is independently —SR, —S(O)₂R, —S(O)₂NR₂, or —S(O)R.

In some embodiments, each instance of R^{G3C} is independently —N(R)C(O)OR, —N(R)C(O)R, or —N(R)C(O)NR₂. In some embodiments, each instance of R^{G3C} is independently —N(R)S(O)₂NR₂ or —N(R)S(O)₂R. In some embodiments, each instance of R^{G3C} is independently —N(R)C(O)OR or —N(R)C(O)R. In some embodiments, each instance of R^{G3C} is independently —N(R)C(O)NR₂ or —N(R)S(O)₂NR₂. In some embodiments, each instance of R^{G3C} is independently —N(R)C(O)OR, —N(R)C(O)R, or —N(R)S(O)₂R.

In some embodiments, each instance of R^{G3C} is independently —NR₂, —N(R)C(O)OR, —N(R)C(O)R, or —N(R)C(O)NR₂. In some embodiments, each instance of R^{G3C} is independently —NR₂, —N(R)C(O)OR, or —N(R)C(O)R. In some embodiments, each instance of R^{G3C} is independently —NR₂, —N(R)C(O)OR, —N(R)C(O)R, or —N(R)S(O)₂R.

In some embodiments, each instance of R^{G3C} is independently an optionally substituted C₁₋₆ aliphatic. In some embodiments, each instance of R^{G3C} is independently an optionally substituted phenyl. In some embodiments, each instance of R^{G3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{G3C} is independently an optionally substituted 5-6 membered monocyclic heteroaryl ring having 1-4 heteroatoms independently selected from nitrogen, oxygen, and sulfur.

In some embodiments, each instance of R^{G3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{G3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{G3C} is independently an optionally substituted C₁₋₆ aliphatic or an optionally substituted phenyl. In some embodiments, each instance of R^{G3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{G3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{G3C} is independently a C₁₋₆ aliphatic. In some embodiments, R^{G3C} is phenyl. In some embodiments, each instance of R^{G3C} is independently a 3-7 membered saturated or partially unsaturated monocyclic heterocyclic ring having 1-2 heteroatoms independently selected from nitrogen, oxygen, and sulfur. In some embodiments, each instance of R^{G3C} is independently a 5-6 membered monocyclic heteroaryl ring having 1-4 heteroatoms independently selected from nitrogen, oxygen, and sulfur.

In some embodiments, each instance of R^{G3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{G3C} is independently phenyl or a 5-6 membered monocyclic heteroaryl ring having 1-4 heteroatoms independently selected from nitrogen, oxygen, and sulfur.

In some embodiments, each instance of R^{G3C} is independently a C₁₋₆ aliphatic or phenyl. In some embodiments, each instance of R^{G3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{G3C} is independently selected from the groups depicted in the compounds in Table 1.

As defined generally above, each instance of R^{M1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{M1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{M1C} is oxo. In some embodiments, R^{M1C} is deuterium. In some embodiments, each instance of R^{M1C} is independently halogen. In some embodiments, R^{M1C} is —CN. In some embodiments, R^{M1C} is —NO₂. In some embodiments, R^{M1C} is —OR. In some embodiments, R^{M1C} is —SR. In some embodiments, R^{M1C} is —NR₂. In some embodiments, R^{M1C} is —S(O)₂R. In some embodiments, R^{M1C} is —S(O)₂NR₂. In some embodiments, R^{M1C} is —S(O)₂F. In some embodiments, R^{M1C} is —S(O)R. In some embodiments, R^{M1C} is —S(O)NR₂. In some embodiments, R^{M1C} is —S(O)(NR)R. In some embodiments, R^{M1C} is —C(O)R. In some embodiments, R^{M1C} is —C(O)OR. In some embodiments, R^{M1C} is —C(O)NR₂. In some embodiments, R^{M1C} is —C(O)N(R)OR. In some embodiments, R^{M1C} is —OC(O)R. In some embodiments, R^{M1C} is —OC(O)NR₂. In some embodiments, R^{M1C} is —N(R)C(O)OR. In some embodiments, R^{M1C} is —N(R)C(O)R. In some embodiments, R^{M1C} is —N(R)C(O)NR₂. In some embodiments, R^{M1C} is —N(R)C(NR)NR₂. In some embodiments, R^{M1C} is —N(R)S(O)₂NR₂. In some embodiments, R^{M1C} is —N(R)S(O)₂R. In some embodiments, R^{M1C} is —P(O)R₂. In some embodiments, R^{M1C} is —P(O)(R)OR. In some embodiments, R^{M1C} is —B(OR)₂.

In some embodiments, each instance of R^{M1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{M1C} is independently halogen, —CN, or —NO₂. In some embodiments, each instance of R^{M1C} is independently —OR, —SR, or —NR₂. In some embodiments, each instance of R^{M1C} is independently —S(O)₂R, —S(O)₂NR₂, —S(O)₂F, —S(O)R, —S(O)NR₂, or —S(O)(NR)R. In some embodiments, each instance of R^{M1C} is independently —C(O)R, —C(O)OR, —C(O)NR₂, or —C(O)N(R)OR. In some embodiments, each instance of R^{M1C} is independently —OC(O)R or —OC(O)NR₂. In some embodiments, each instance of R^{M1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{M1C} is independently —P(O)R₂ or —P(O)(R)OR.

In some embodiments, each instance of R^{M1C} is independently —OR, —OC(O)R, or —OC(O)NR₂. In some embodiments, each instance of R^{M1C} is independently —SR, —S(O)₂R, —S(O)₂NR₂, —S(O)₂F, —S(O)R, —S(O)NR₂, or —S(O)(NR)R. In some embodiments, each instance of R^{M1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{M1C} is independently —S(O)₂R, —S(O)₂NR₂, or —S(O)₂F. In some embodiments, each instance of R^{M1C} is independently —S(O)R, —S(O)NR₂, or —S(O)(NR)R. In some embodiments, each instance of R^{M1C} is independently —SR, —S(O)₂R, or —S(O)R. In some embodiments, each instance of R^{M1C} is independently —S(O)₂NR₂, —S(O)NR₂, or —S(O)(NR)R. In some embodiments, each instance of R^{M1C} is independently —S(O)₂NR₂ or —S(O)NR₂. In some embodiments, each instance of R^{M1C} is independently —SR, —S(O)₂R, —S(O)₂NR₂, or —S(O)R.

In some embodiments, each instance of R^{M1C} is independently —N(R)C(O)OR, —N(R)C(O)R, or —N(R)C(O)NR₂. In some embodiments, each instance of R^{M1C} is independently —N(R)S(O)₂NR₂ or —N(R)S(O)₂R. In some embodiments, each instance of R^{M1C} is independently —N(R)C(O)OR or —N(R)C(O)R. In some embodiments, each instance of R^{M1C} is independently —N(R)C(O)NR₂ or —N(R)S(O)₂NR₂. In some embodiments, each instance of R^{M1C} is independently —N(R)C(O)OR, —N(R)C(O)R, or —N(R)S(O)₂R.

In some embodiments, each instance of R^{M1C} is independently —NR₂, —N(R)C(O)OR, —N(R)C(O)R, or —N(R)C(O)NR₂. In some embodiments, each instance of R^{M1C} is independently —NR₂, —N(R)C(O)OR, or —N(R)C(O)R. In some embodiments, each instance of R^{M1C} is independently —NR₂, —N(R)C(O)OR, —N(R)C(O)R, or —N(R)S(O)₂R.

In some embodiments, each instance of R^{M1C} is independently an optionally substituted C₁₋₆ aliphatic. In some embodiments, each instance of R^{M1C} is independently an optionally substituted phenyl. In some embodiments, each instance of R^{M1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{M1C} is independently an optionally substituted 5-6 membered monocyclic heteroaryl ring having 1-4 heteroatoms independently selected from nitrogen, oxygen, and sulfur.

In some embodiments, each instance of R^{M1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{M1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{M1C} is independently an optionally substituted C₁₋₆ aliphatic or an optionally substituted phenyl. In some embodiments, each instance of R^{M1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{M1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M1C} is independently a C₁₋₆ aliphatic. In some embodiments, R^{M1C} is phenyl. In some embodiments, each instance of R^{M1C} is independently a 3-7 membered saturated or partially unsaturated monocyclic heterocyclic ring having 1-2 heteroatoms independently selected from nitrogen, oxygen, and sulfur. In some embodiments, each instance of R^{M1C} is independently a 5-6 membered monocyclic heteroaryl ring having 1-4 heteroatoms independently selected from nitrogen, oxygen, and sulfur.

In some embodiments, each instance of R^{M1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{M1C} is independently phenyl or a 5-6 membered monocyclic heteroaryl ring having 1-4 heteroatoms independently selected from nitrogen, oxygen, and sulfur.

In some embodiments, each instance of R^{M1C} is independently a C₁₋₆ aliphatic or phenyl. In some embodiments, each instance of R^{M1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{M1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{M1C} is independently selected from the groups depicted in the compounds in Table 1.

As defined generally above, each instance of R^{M2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{M2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{M2C} is oxo. In some embodiments, R^{M2C} is deuterium. In some embodiments, each instance of R^{M2C} is independently halogen. In some embodiments, R^{M2C} is —CN. In some embodiments, R^{M2C} is —NO₂. In some embodiments, R^{M2C} is —OR. In some embodiments, R^{M2C} is —SR. In some embodiments, R^{M2C} is —NR₂. In some embodiments, R^{M2C} is —S(O)₂R. In some embodiments, R^{M2C} is —S(O)₂NR₂. In some embodiments, R^{M2C} is —S(O)₂F. In some embodiments, R^{M2C} is —S(O)R. In some embodiments, R^{M2C} is —S(O)NR₂. In some embodiments, R^{M2C} is —S(O)(NR)R. In some embodiments, R^{M2C} is —C(O)R. In some embodiments, R^{M2C} is —C(O)OR. In some embodiments, R^{M2C} is —C(O)NR₂. In some embodiments, R^{M2C} is —C(O)N(R)OR. In some embodiments, R^{M2C} is —OC(O)R. In some embodiments, R^{M2C} is —OC(O)NR₂. In some embodiments, R^{M2C} is —N(R)C(O)OR. In some embodiments, R^{M2C} is —N(R)C(O)R. In some embodiments, R^{M2C} is —N(R)C(O)NR₂. In some embodiments, R^{M2C} is —N(R)C(NR)NR₂. In some embodiments, R^{M2C} is —N(R)S(O)₂NR₂. In some embodiments, R^{M2C} is —N(R)S(O)₂R. In some embodiments, R^{M2C} is —P(O)R₂. In some embodiments, R^{M2C} is —P(O)(R)OR. In some embodiments, R^{M2C} is —B(OR)₂.

In some embodiments, each instance of R^{M2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{M2C} is independently halogen, —CN, or —NO². In some embodiments, each instance of R^{M2C} is independently —OR, —SR, or —NR₂. In some embodiments, each instance of R^{M2C} is independently —S(O)₂R, —S(O)₂NR₂, —S(O)₂F, —S(O)R, —S(O)NR₂, or —S(O)(NR)R. In some embodiments, each instance of R^{M2C} is independently —C(O)R, —C(O)OR, —C(O)NR₂, or —C(O)N(R)OR. In some embodiments, each instance of R^{M2C} is independently —OC(O)R or —OC(O)NR₂. In some embodiments, each instance of R^{M2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{M2C} is independently —P(O)R₂ or —P(O)(R)OR.

In some embodiments, each instance of R^{M2C} is independently —OR, —OC(O)R, or —OC(O)NR₂. In some embodiments, each instance of R^{M2C} is independently —SR, —S(O)₂R, —S(O)₂NR₂, —S(O)₂F, —S(O)R, —S(O)NR₂, or —S(O)(NR)R. In some embodiments, each instance of R^{M2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{M2C} is independently —S(O)₂R, —S(O)₂NR₂, or —S(O)₂F. In some embodiments, each instance of R^{M2C} is independently —S(O)R, —S(O)NR₂, or —S(O)(NR)R. In some embodiments, each instance of R^{M2C} is independently —SR, —S(O)₂R, or —S(O)R. In some embodiments, each instance of R^{M2C} is independently —S(O)₂NR₂, —S(O)NR₂, or —S(O)(NR)R. In some embodiments, each instance of R^{M2C} is independently —S(O)₂NR₂ or —S(O)NR₂. In some embodiments, each instance of R^{M2C} is independently —SR, —S(O)₂R, —S(O)₂NR₂, or —S(O)R.

In some embodiments, each instance of R^{M2C} is independently —N(R)C(O)OR, —N(R)C(O)R, or —N(R)C(O)NR₂. In some embodiments, each instance of R^{M2C} is independently —N(R)S(O)₂NR₂ or —N(R)S(O)₂R. In some embodiments, each instance of R^{M2C} is independently —N(R)C(O)OR or —N(R)C(O)R. In some embodiments, each instance of R^{M2C} is independently —N(R)C(O)NR₂ or —N(R)S(O)₂NR₂. In some embodiments, each instance of R^{M2C} is independently —N(R)C(O)OR, —N(R)C(O)R, or —N(R)S(O)₂R.

In some embodiments, each instance of R^{M2C} is independently —NR₂, —N(R)C(O)OR, —N(R)C(O)R, or —N(R)C(O)NR₂. In some embodiments, each instance of R^{M2C} is independently —NR₂, —N(R)C(O)OR, or —N(R)C(O)R. In some embodiments, each instance of R^{M2C} is independently —NR₂, —N(R)C(O)OR, —N(R)C(O)R, or —N(R)S(O)₂R.

In some embodiments, each instance of R^{M2C} is independently an optionally substituted C₁₋₆ aliphatic. In some embodiments, each instance of R^{M2C} is independently an optionally substituted phenyl. In some embodiments, each instance of R^{M2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{M2C} is independently an optionally substituted 5-6 membered monocyclic heteroaryl ring having 1-4 heteroatoms independently selected from nitrogen, oxygen, and sulfur.

In some embodiments, each instance of R^{M2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{M2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{M2C} is independently an optionally substituted C₁₋₆ aliphatic or an optionally substituted phenyl. In some embodiments, each instance of R^{M2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{M2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{M2C} is independently a C₁₋₆ aliphatic. In some embodiments, R^{M2C} is phenyl. In some embodiments, each instance of R^{M2C} is independently a 3-7 membered saturated or partially unsaturated monocyclic heterocyclic ring having 1-2 heteroatoms independently selected from nitrogen, oxygen, and sulfur. In some embodiments, each instance of R^{M2C} is independently a 5-6 membered monocyclic heteroaryl ring having 1-4 heteroatoms independently selected from nitrogen, oxygen, and sulfur.

In some embodiments, each instance of R^{M2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{M2C} is independently phenyl or a 5-6 membered monocyclic heteroaryl ring having 1-4 heteroatoms independently selected from nitrogen, oxygen, and sulfur.

In some embodiments, each instance of R^{M2C} is independently a C₁₋₆ aliphatic or phenyl. In some embodiments, each instance of R^{M2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{M2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{M2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{M2C} is independently halogen, —CN, —OH, —O-(optionally substituted C₁₋₃ aliphatic), or an optionally substituted C₁₋₃ aliphatic. In some embodiments, each instance of R^{M2C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{M2C} is independently fluorine, chlorine, —OH, —OCH₃, —OCF₃, —CH₃, —CHF₂, or —CF₃. In some embodiments, each instance of R^{M2C} is independently fluorine or —OH.

In some embodiments, each instance of R^{M2C} is independently oxo, deuterium, halogen, —CN, —OH, —O-(optionally substituted C₁₋₃ aliphatic), or an optionally substituted C₁₋₃ aliphatic. In some embodiments, each instance of R^{M2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{M2C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{M2C} is independently oxo, deuterium, fluorine, chlorine, —CN, —OH, —OCH₃, —OCF₃, —CH₃, —CHF₂, or —CF₃. In some embodiments, each instance of R^{M2C} is independently oxo, deuterium, —CN, fluorine, or —OH. In some embodiments, each instance of R^{M2C} is independently oxo, deuterium, —CN, —CH₃, or —CHF₂. In some embodiments, each instance of R^{M2C} is independently deuterium, —CN, —CH₃, or —CHF₂.

In some embodiments, each instance of R^{M2C} is independently oxo, halogen, —CN, —OH, —O-(optionally substituted C₁₋₃ aliphatic), or an optionally substituted C₁₋₃ aliphatic. In some embodiments, each instance of R^{M2C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{M2C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{M2C} is independently oxo, fluorine, chlorine, —CN, —OH, —OCH₃, —OCF₃, —CH₃, —CHF₂, or —CF₃. In some embodiments, each instance of R^{M2C} is independently oxo, —CN, fluorine, or —OH. In some embodiments, each instance of R^{M2C} is independently oxo, —CN, —CH₃, or —CHF₂. In some embodiments, each instance of R^{M2C} is independently —CN, —CH₃, or —CHF₂.

In some embodiments, each instance of R^{M2C} is independently selected from the groups depicted in the compounds in Table 1.

As defined generally above, each instance of R^{L3C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L3C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{L3C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{L3C} is oxo. In some embodiments, R^{L3C} is deuterium. In some embodiments, each instance of R^{L3C} is independently halogen. In some embodiments, R^{L3C} is —CN. In some embodiments, R^{L3C} is —NO₂. In some embodiments, R^{L3C} is —OR. In some embodiments, R^{L3C} is —SR. In some embodiments, R^{L3C} is —NR₂. In some embodiments, R^{L3C} is —S(O)₂R. In some embodiments, R^{L3C} is —S(O)₂NR₂. In some embodiments, R^{L3C} is —S(O)₂F. In some embodiments, R^{L3C} is —S(O)R. In some embodiments, R^{L3C} is —S(O)NR₂. In some embodiments, R^{L3C} is —S(O)(NR)R. In some embodiments, R^{L3C} is —C(O)R. In some embodiments, R^{L3C} is —C(O)OR. In some embodiments, R^{L3C} is —C(O)NR₂. In some embodiments, R^{L3C} is —C(O)N(R)OR. In some embodiments, R^{L3C} is —OC(O)R. In some embodiments, R^{L3C} is —OC(O)NR₂. In some embodiments, R^{L3C} is —N(R)C(O)OR. In some embodiments, R^{L3C} is —N(R)C(O)R. In some embodiments, R^{L3C} is —N(R)C(O)NR₂. In some embodiments, R^{L3C} is —N(R)C(NR)NR₂. In some embodiments, R^{L3C} is —N(R)S(O)₂NR₂. In some embodiments, R^{L3C} is —N(R)S(O)₂R. In some embodiments, R^{L3C} is —P(O)R₂. In some embodiments, R^{L3C} is —P(O)(R)OR. In some embodiments, R^{L3C} is —B(OR)₂.

In some embodiments, each instance of R^{L3C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{L3C} is independently halogen, —CN, or —NO₂. In some embodiments, each instance of R^{L3C} is independently —OR, —SR, or —NR₂. In some embodiments, each instance of R^{L3C} is independently —S(O)₂R, —S(O)₂NR₂, —S(O)₂F, —S(O)R, —S(O)NR₂, or —S(O)(NR)R. In some embodiments, each instance of R^{L3C} is independently —C(O)R, —C(O)OR, —C(O)NR₂, or —C(O)N(R)OR. In some embodiments, each instance of R^{L3C} is independently —OC(O)R or —OC(O)NR₂. In some embodiments, each instance of R^{L3C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{L3C} is independently —P(O)R₂ or —P(O)(R)OR.

In some embodiments, each instance of R^{L3C} is independently —OR, —OC(O)R, or —OC(O)NR₂. In some embodiments, each instance of R^{L3C} is independently —SR, —S(O)₂R, —S(O)₂NR₂, —S(O)₂F, —S(O)R, —S(O)NR₂, or —S(O)(NR)R. In some embodiments, each instance of R^{L3C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{L3C} is independently —S(O)₂R, —S(O)₂NR₂, or —S(O)₂F. In some embodiments, each instance of R^{L3C} is independently —S(O)R, —S(O)NR₂, or —S(O)(NR)R. In some embodiments, each instance of R^{L3C} is independently —SR, —S(O)₂R, or —S(O)R. In some embodiments, each instance of R^{L3C} is independently —S(O)₂NR₂, —S(O)NR₂, or —S(O)(NR)R. In some embodiments, each instance of R^{L3C} is independently —S(O)₂NR₂ or —S(O)NR₂. In some embodiments, each instance of R^{L3C} is independently —SR, —S(O)₂R, —S(O)₂NR₂, or —S(O)R.

In some embodiments, each instance of R^{L3C} is independently —N(R)C(O)OR, —N(R)C(O)R, or —N(R)C(O)NR₂. In some embodiments, each instance of R^{L3C} is independently —N(R)S(O)₂NR₂ or —N(R)S(O)₂R. In some embodiments, each instance of R^{L3C} is independently —N(R)C(O)OR or —N(R)C(O)R. In some embodiments, each instance of R^{L3C} is independently —N(R)C(O)NR₂ or —N(R)S(O)₂NR₂. In some embodiments, each instance of R^{L3C} is independently —N(R)C(O)OR, —N(R)C(O)R, or —N(R)S(O)₂R.

In some embodiments, each instance of R^{L3C} is independently —NR₂, —N(R)C(O)OR, —N(R)C(O)R, or —N(R)C(O)NR₂. In some embodiments, each instance of R^{L3C} is independently —NR₂, —N(R)C(O)OR, or —N(R)C(O)R. In some embodiments, each instance of R^{L3C} is independently —NR₂, —N(R)C(O)OR, —N(R)C(O)R, or —N(R)S(O)₂R.

In some embodiments, each instance of R^{L3C} is independently an optionally substituted C₁₋₆ aliphatic. In some embodiments, each instance of R^{L3C} is independently an optionally substituted phenyl. In some embodiments, each instance of R^{L3C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{L3C} is independently an optionally substituted 5-6 membered monocyclic heteroaryl ring having 1-4 heteroatoms independently selected from nitrogen, oxygen, and sulfur.

In some embodiments, each instance of R^{L3C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{L3C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{L3C} is independently an optionally substituted C₁₋₆ aliphatic or an optionally substituted phenyl. In some embodiments, each instance of R^{L3C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{L3C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{L3C} is independently a C₁₋₆ aliphatic. In some embodiments, R^{L3C} is phenyl. In some embodiments, each instance of R^{L3C} is independently a 3-7 membered saturated or partially unsaturated monocyclic heterocyclic ring having 1-2 heteroatoms independently selected from nitrogen, oxygen, and sulfur. In some embodiments, each instance of R^{L3C} is independently a 5-6 membered monocyclic heteroaryl ring having 1-4 heteroatoms independently selected from nitrogen, oxygen, and

In some embodiments, each instance of R^{L3C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{L3C} is independently phenyl or a 5-6 membered monocyclic heteroaryl ring having 1-4 heteroatoms independently selected from nitrogen, oxygen, and sulfur.

In some embodiments, each instance of R^{L3C} is independently a C₁₋₆ aliphatic or phenyl. In some embodiments, each instance of R^{L3C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L3C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{L3C} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, z¹ is 0, 1, 2, 3, or 4. In some embodiments, z¹ is 0. In some embodiments, z¹ is 1. In some embodiments, z¹ is 2. In some embodiments, z¹ is 3. In some embodiments, z¹ is 4. In some embodiments, z¹ is 0 or 1. In some embodiments, z¹ is 0, 1, or 2. In some embodiments, z¹ is 0, 1, 2, or 3. In some embodiments, z¹ is 1 or 2. In some embodiments, z¹ is 1, 2, or 3. In some embodiments, z¹ is 1, 2, 3, or 4. In some embodiments, z¹ is 2 or 3. In some embodiments, z¹ is 2, 3, or 4. In some embodiments, z¹ is 3 or 4. In some embodiments, z¹ is selected from the values represented in the compounds in Table 1.

As defined generally above, z² is 0, 1, 2, 3, or 4. In some embodiments, z² is 0. In some embodiments, z² is 1. In some embodiments, z² is 2. In some embodiments, z² is 3. In some embodiments, z² is 4. In some embodiments, z² is 0 or 1. In some embodiments, z² is 0, 1, or 2. In some embodiments, z² is 0, 1, 2, or 3. In some embodiments, z² is 1 or 2. In some embodiments, z² is 1, 2, or 3. In some embodiments, z² is 1, 2, 3, or 4. In some embodiments, z² is 2 or 3. In some embodiments, z² is 2, 3, or 4. In some embodiments, z² is 3 or 4. In some embodiments, z² is selected from the values represented in the compounds in Table 1.

As defined generally above, z³ is 0, 1, 2, 3, or 4. In some embodiments, z³ is 0. In some embodiments, z³ is 1. In some embodiments, z³ is 2. In some embodiments, z³ is 3. In some embodiments, z³ is 4. In some embodiments, z³ is 0 or 1. In some embodiments, z³ is 0, 1, or 2. In some embodiments, z³ is 0, 1, 2, or 3. In some embodiments, z³ is 1 or 2. In some embodiments, z³ is 1, 2, or 3. In some embodiments, z³ is 1, 2, 3, or 4. In some embodiments, z³ is 2 or 3. In some embodiments, z³ is 2, 3, or 4. In some embodiments, z³ is 3 or 4. In some embodiments, z³ is selected from the values represented in the compounds in Table 1.

As defined generally above, z⁴ is 0, 1, 2, 3, or 4. In some embodiments, z⁴ is 0. In some embodiments, z⁴ is 1. In some embodiments, z⁴ is 2. In some embodiments, z⁴ is 3. In some embodiments, z⁴ is 4. In some embodiments, z⁴ is 0 or 1. In some embodiments, z⁴ is 0, 1, or 2. In some embodiments, z⁴ is 0, 1, 2, or 3. In some embodiments, z⁴ is 1 or 2. In some embodiments, z⁴ is 1, 2, or 3. In some embodiments, z⁴ is 1, 2, 3, or 4. In some embodiments, z⁴ is 2 or 3. In some embodiments, z⁴ is 2, 3, or 4. In some embodiments, z⁴ is 3 or 4. In some embodiments, z⁴ is selected from the values represented in the compounds in Table 1.

As defined generally above, z⁵ is 0, 1, 2, 3, or 4. In some embodiments, z⁵ is 0. In some embodiments, z⁵ is 1. In some embodiments, z⁵ is 2. In some embodiments, z⁵ is 3. In some embodiments, z⁵ is 4. In some embodiments, z⁵ is 0 or 1. In some embodiments, z⁵ is 0, 1, or 2. In some embodiments, z⁵ is 0, 1, 2, or 3. In some embodiments, z⁵ is 1 or 2. In some embodiments, z⁵ is 1, 2, or 3. In some embodiments, z⁵ is 1, 2, 3, or 4. In some embodiments, z⁵ is 2 or 3. In some embodiments, z⁵ is 2, 3, or 4. In some embodiments, z⁵ is 3 or 4. In some embodiments, z⁵ is selected from the values represented in the compounds in Table 1.

As defined generally above, z⁶ is 0, 1, 2, 3, or 4. In some embodiments, z⁶ is 0. In some embodiments, z⁶ is 1. In some embodiments, z⁶ is 2. In some embodiments, z⁶ is 3. In some embodiments, z⁶ is 4. In some embodiments, z⁶ is 0 or 1. In some embodiments, z⁶ is 0, 1, or 2. In some embodiments, z⁶ is 0, 1, 2, or 3. In some embodiments, z⁶ is 1 or 2. In some embodiments, z⁶ is 1, 2, or 3. In some embodiments, z⁶ is 1, 2, 3, or 4. In some embodiments, z⁶ is 2 or 3. In some embodiments, z⁶ is 2, 3, or 4. In some embodiments, z⁶ is 3 or 4. In some embodiments, z⁶ is selected from the values represented in the compounds in Table 1.

As defined generally above, z⁷ is 0, 1, 2, 3, or 4. In some embodiments, z⁷ is 0. In some embodiments, z⁷ is 1. In some embodiments, z⁷ is 2. In some embodiments, z⁷ is 3. In some embodiments, z⁷ is 4. In some embodiments, z⁷ is 0 or 1. In some embodiments, z⁷ is 0, 1, or 2. In some embodiments, z⁷ is 0, 1, 2, or 3. In some embodiments, z⁷ is 1 or 2. In some embodiments, z⁷ is 1, 2, or 3. In some embodiments, z⁷ is 1, 2, 3, or 4. In some embodiments, z⁷ is 2 or 3. In some embodiments, z⁷ is 2, 3, or 4. In some embodiments, z⁷ is 3 or 4. In some embodiments, z⁷ is selected from the values represented in the compounds in Table 1.

As defined generally above, z⁸ is 0, 1, 2, 3, or 4. In some embodiments, z⁸ is 0. In some embodiments, z⁸ is 1. In some embodiments, z⁸ is 2. In some embodiments, z⁸ is 3. In some embodiments, z⁸ is 4. In some embodiments, z⁸ is 0 or 1. In some embodiments, z⁸ is 0, 1, or 2. In some embodiments, z⁸ is 0, 1, 2, or 3. In some embodiments, z⁸ is 1 or 2. In some embodiments, z⁸ is 1, 2, or 3. In some embodiments, z⁸ is 1, 2, 3, or 4. In some embodiments, z⁸ is 2 or 3. In some embodiments, z⁸ is 2, 3, or 4. In some embodiments, z⁸ is 3 or 4. In some embodiments, z⁸ is selected from the values represented in the compounds in Table 1.

In some embodiments, the present disclosure provides a compound of formula I-d, wherein G¹ is CH, G² is C—R^{G2}, and each of G³ and G⁴ is CH or CR⁸, forming a compound of formula I-d1 or I-d2:

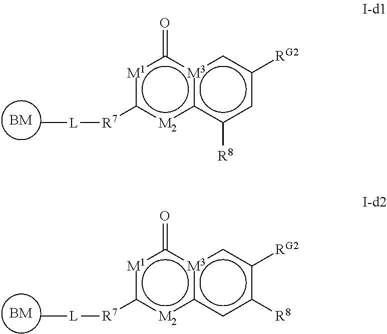

or a pharmaceutically acceptable salt thereof, wherein each of BM, L, R⁷, R⁸, R^{G2}, M¹, M², and M³ is as defined in embodiments and classes and subclasses herein.

In some embodiments, the present disclosure provides a compound of formula I-d1 or I-d2 wherein M¹ is CH, M² is O, and M³ is C, forming a compound of formula I-d3 or I-d4:

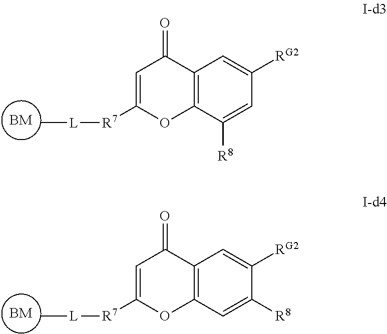

or a pharmaceutically acceptable salt thereof, wherein each of BM, L, R⁷, R⁸, and R^{G2} is as defined in embodiments and classes and subclasses herein.

In some embodiments, the present disclosure provides a compound of formula I-d1 or I-d2 wherein M¹ is C(R^{M1}), M² is O, and M³ is C, forming a compound of formula I-d5 or I-d6:

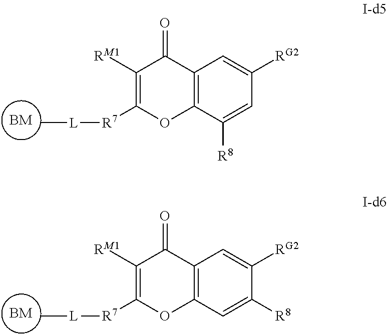

or a pharmaceutically acceptable salt thereof, wherein each of BM, L, R⁷, R⁸, R^{G2}, and R^{M1} is as defined in embodiments and classes and subclasses herein.

In some embodiments, the present disclosure provides a compound of formula I-d1 or I-d2 wherein M¹ is CH, M² is N, and M³ is N, forming a compound of formula I-d7 or I-d8:

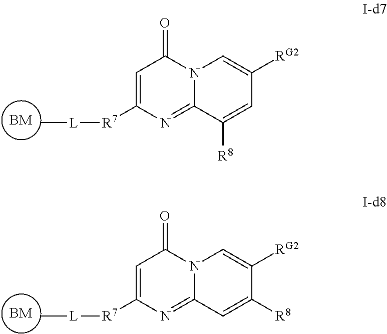

or a pharmaceutically acceptable salt thereof, wherein each of BM, L, R⁷, R⁸, and R^{G2} is as defined in embodiments and classes and subclasses herein.

In some embodiments, the present disclosure provides a compound of formula I-d1 or I-d2 wherein M¹ is C(R^{M1}), M² is N, and M³ is N, forming a compound of formula I-d9 or I-d10:

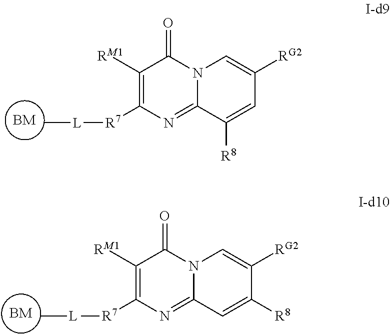

or a pharmaceutically acceptable salt thereof, wherein each of BM, L, R⁷, R⁸, R^{G2}, and R^{M1} is as defined in embodiments and classes and subclasses herein.

In some embodiments, the present disclosure provides a compound of formula I-d1 or I-d2 wherein M¹ is N(R^{M1}), M² is N, and M³ is C, forming a compound of formula I-d11 or I-d12:

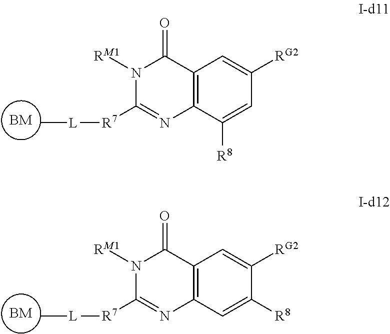

or a pharmaceutically acceptable salt thereof, wherein each of BM, L, R⁷, R⁸, R^{G2}, and R^{M1} is as defined in embodiments and classes and subclasses herein.

### Linker (L)

As defined generally above and described herein, L is a bivalent moiety that connects PIK to BM. In some embodiments, L is a bivalent moiety that connects PIK to LBM. In some embodiments, L is a bivalent moiety that connects PIK to PIK2.

In some embodiments, L is a covalent bond or a bivalent, saturated or unsaturated, straight or branched C₁₋₅₀ hydrocarbon chain, wherein 0-10 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—,

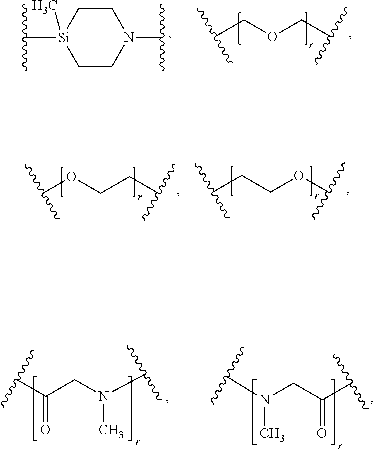

wherein: each -Cy- is independently an optionally substituted bivalent ring selected from phenylene, an 8-10 membered bicyclic arylene, a 3-7 membered saturated or partially unsaturated carbocyclylene, a 3-12 membered saturated or partially unsaturated spiro carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, a 3-12 membered saturated or partially unsaturated spiro heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, and wherein r is 0, 1, 2, 3, 4, 5, 6, 7, 8, 9, or 10.

In some embodiments, one or more instances of -Cy- is an optionally substituted bivalent phenylene. In some embodiments, one or more instances of -Cy- is an optionally substituted 8-10 membered bicyclic arylene. In some embodiments, one or more instances of -Cy- is independently an optionally substituted 4-7 membered saturated or partially unsaturated carbocyclylene. In some embodiments, one or more instances of -Cy- is independently an optionally substituted 3-12 membered saturated or partially unsaturated spiro carbocyclylene. In some embodiments, one or more instances of -Cy- is independently an optionally substituted 8-10 membered bicyclic saturated or partially unsaturated carbocyclylene. In some embodiments, one or more instances of -Cy- is independently an optionally substituted 4-7 membered saturated or partially unsaturated heterocyclylene having 1-2 heteroatoms independently selected from nitrogen, oxygen, and sulfur. In some embodiments, one or more instances of -Cy- is independently an optionally substituted 4-12 membered saturated or partially unsaturated spiro heterocyclylene having 1-2 heteroatoms independently selected from nitrogen, oxygen, and sulfur. In some embodiments, one or more instances of -Cy- is an optionally substituted 8-10 membered bicyclic saturated or partially unsaturated heterocyclylene having 1-2 heteroatoms independently selected from nitrogen, oxygen, and sulfur. In some embodiments, one or more instances of -Cy- is independently an optionally substituted 5-6 membered heteroarylene having 1-4 heteroatoms independently selected from nitrogen, oxygen, and sulfur. In some embodiments, one or more instances of -Cy- is independently an optionally substituted 8-10 membered bicyclic heteroarylene having 1-5 heteroatoms independently selected from nitrogen, oxygen, or sulfur.

In some embodiments, -Cy- is

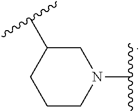 [SMILES: *C1CCCN(*)C1]

In some embodiments, -Cy- is

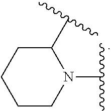 [SMILES: *C1CCCCN1*]

In some embodiments, -Cy- is N

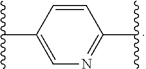 [SMILES: *c1ccc(*)nc1]

In some embodiments, -Cy- is

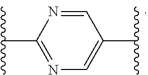 [SMILES: *c1cnc(*)nc1]

In some embodiments, -Cy- is

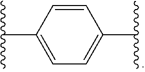 [SMILES: *c1ccc(*)cc1]

some embodiments, -Cy- is

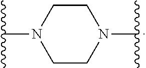 [SMILES: *N1CCN(*)CC1]

In some embodiments, -Cy- is

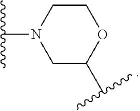 [SMILES: *C1CN(*)CCO1]

In some embodiments, -Cy- is

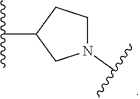 [SMILES: *C1CCN(*)C1]

In some embodiments, -Cy- is

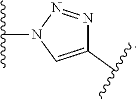 [SMILES: *c1cn(*)nn1]

In some embodiments, -Cy- is

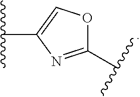 [SMILES: *c1coc(*)n1]

In some embodiments, -Cy- is

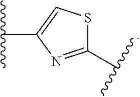 [SMILES: *c1csc(*)n1]

In some embodiments, -Cy- is

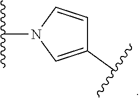 [SMILES: *c1ccn(*)c1]

In some embodiments, -Cy- is

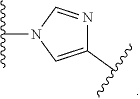 [SMILES: *c1cn(*)cn1]

In some embodiments, -Cy- is

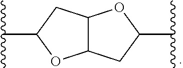 [SMILES: *C1CC2OC(*)CC2O1]

In some embodiments, -Cy- is

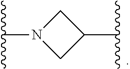 [SMILES: *C1CN(*)C1]

In some embodiments, -Cy- is

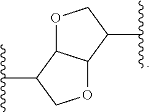 [SMILES: *C1COC2C(*)COC12]

In some embodiments, -Cy- is

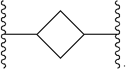 [SMILES: *C1CC(*)C1]

In some embodiments, -Cy- is

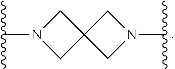 [SMILES: *N1CC2(C1)CN(*)C2]

In some embodiments, -Cy- is

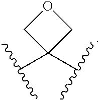 [SMILES: *C1(*)COC1]

In some embodiments, -Cy- is

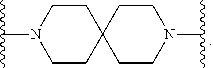 [SMILES: *N1CCC2(CC1)CCN(*)CC2]

In some embodiments, -Cy- is

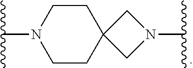 [SMILES: *N1CCC2(CC1)CN(*)C2]

In some embodiments, -Cy- is

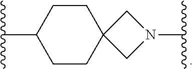 [SMILES: *C1CCC2(CC1)CN(*)C2]

In some embodiments, -Cy- is

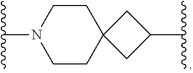 [SMILES: *C1CC2(CCN(*)CC2)C1]

In some embodiments, -Cy- is

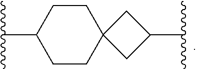 [SMILES: *C1CCC2(CC1)CC(*)C2]

In some embodiments, -Cy- is

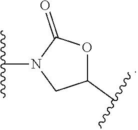 [SMILES: *C1CN(*)C(=O)O1]

In some embodiments, -Cy- is

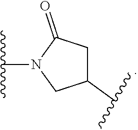 [SMILES: *C1CC(=O)N(*)C1]

In some embodiments, -Cy- is

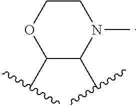 [SMILES: *C1OCCN(C)C1*]

In some embodiments, -Cy- is

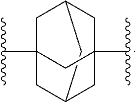 [SMILES: *C12CC3CC(C1)CC(*)(C3)C2]

In some embodiments, -Cy- is

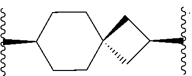

In some embodiments, -Cy- is

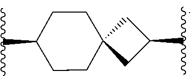

In some embodiments, -Cy- is

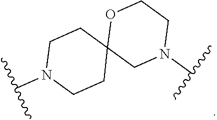 [SMILES: *N1CCC2(CC1)CN(*)CCO2]

In some embodiments, -Cy- is

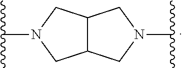 [SMILES: *N1CC2CN(*)CC2C1]

In some embodiments, -Cy- is

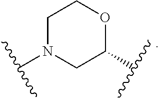 [SMILES: *[C@@H]1CN(*)CCO1]

In some embodiments, -Cy- is

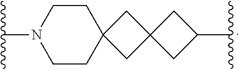 [SMILES: *C1CC2(C1)CC1(CCN(*)CC1)C2]

In some embodiments, -Cy- is

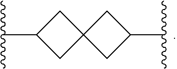 [SMILES: *C1CC2(C1)CC(*)C2]

In some embodiments, -Cy- is selected from the groups depicted in the compounds in Table 1.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from the values depicted in the compounds in Table 1.

In some embodiments, L is a bivalent, saturated or unsaturated, straight or branched C₁₋₂₀ hydrocarbon chain, wherein 0-7 methylene units of L are independently replaced by —C(H)F—, —CF₂—, —O—, —N(R)—, —S—, —C(O)—, —S(O)—, —S(O)₂—, —N(R)S(O)₂—, —S(O)₂N(R)—, and 0 or 1 methylene unit of L is replaced by phenylene, a 3-7 membered saturated or partially unsaturated carbocyclylene, or a 3-7 membered saturated or partially unsaturated heterocyclylene having 1-2 heteroatoms independently selected from nitrogen, oxygen, and sulfur.

In some embodiments, L is a bivalent, saturated or unsaturated, straight or branched C₁₋₂₀ hydrocarbon chain, wherein 0-7 methylene units of L are independently replaced by —O—, —N(R)—, or —C(O)—. In some embodiments, L is a bivalent, saturated or unsaturated, straight or branched C₁₋₂₀ hydrocarbon chain, wherein 0-7 methylene units of L are independently replaced by —O—, —N(H)—, —N(C₁₋₆ aliphatic)-, or —C(O)—.

In some embodiments, L is

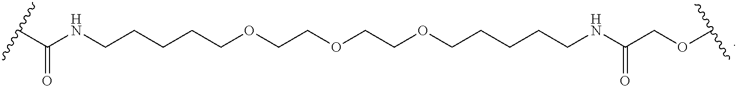 [SMILES: *OCC(=O)NCCCCCOCCOCCOCCCCCNC(*)=O]

In some embodiments, L is

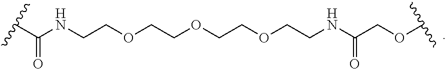 [SMILES: *OCC(=O)NCCOCCOCCOCCNC(*)=O]

In some embodiments, L is

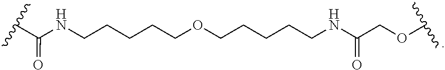 [SMILES: *OCC(=O)NCCCCCOCCCCCNC(*)=O]

In some embodiments, L is

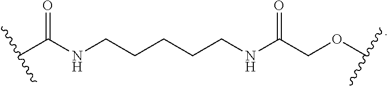 [SMILES: *OCC(=O)NCCCCCNC(*)=O]

In some embodiments, L is

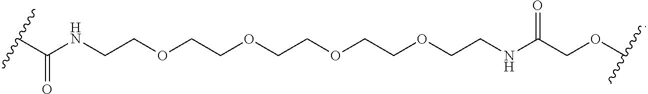 [SMILES: *OCC(=O)NCCOCCOCCOCCOCCNC(*)=O ; "."]

In some embodiments, L is

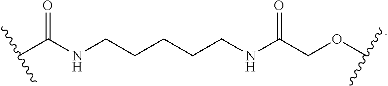 [SMILES: *OCC(=O)NCCCCCNC(*)=O]

In some embodiments, L is

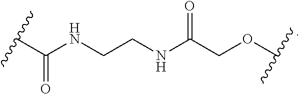 [SMILES: *OCC(=O)NCCNC(*)=O]

In some embodiments, L is

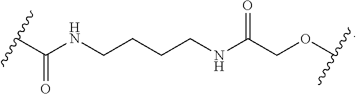 [SMILES: *OCC(=O)NCCCCNC(*)=O]

In some embodiments, L is

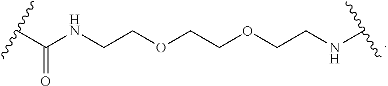 [SMILES: *NCCOCCOCCNC(*)=O]

In some embodiments, L is

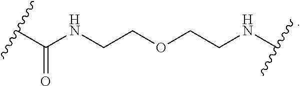 [SMILES: *NCCOCCNC(*)=O]

In some embodiments, L is

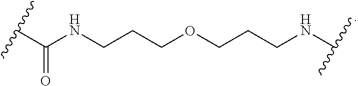 [SMILES: *NCCCOCCCNC(*)=O]

In some embodiments, L is

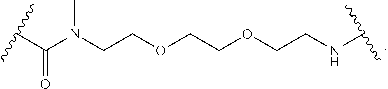 [SMILES: *NCCOCCOCCN(C)C(*)=O]

In some embodiments, L is

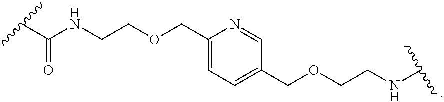 [SMILES: *NCCOCc1ccc(COCCNC(*)=O)nc1]

In some embodiments, L is

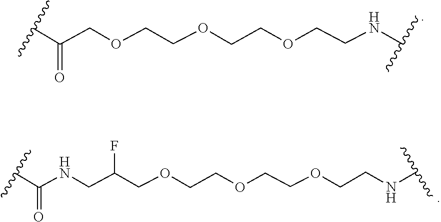 [SMILES: *NCCOCCOCCOCC(*)=O ; *NCCOCCOCCOCC(F)CNC(*)=O]

In some embodiments, L is

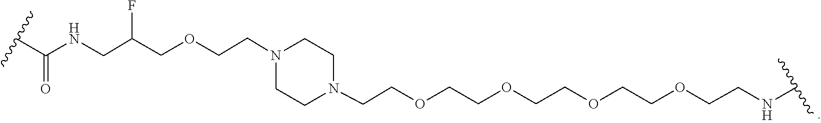 [SMILES: *NCCOCCOCCOCCOCCN1CCN(CCOCC(F)CNC(*)=O)CC1]

In some embodiments, L is

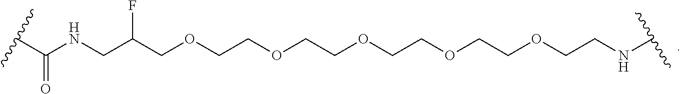 [SMILES: *NCCOCCOCCOCCOCCOCC(F)CNC(*)=O]

In some embodiments, L is

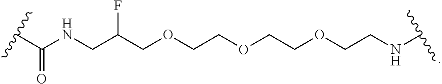 [SMILES: *NCCOCCOCCOCC(F)CNC(*)=O]

In some embodiments, L is

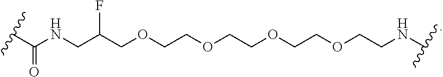 [SMILES: *NCCOCCOCCOCCOCC(F)CNC(*)=O]

In some embodiments, L is

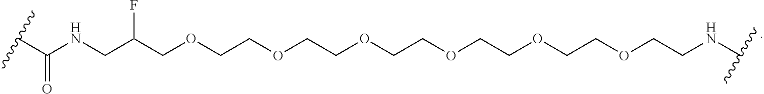 [SMILES: *NCCOCCOCCOCCOCCOCCOCC(F)CNC(*)=O]

In some embodiments, L is

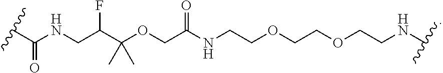 [SMILES: *NCCOCCOCCNC(=O)COC(C)(C)C(F)CNC(*)=O]

In some embodiments, L is

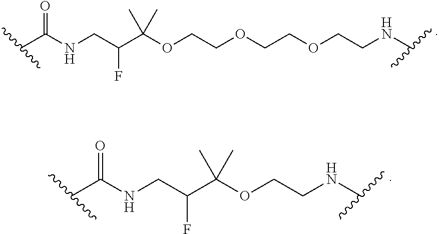 [SMILES: *NCCOCCOCCOC(C)(C)C(F)CNC(*)=O ; *NCCOC(C)(C)C(F)CNC(*)=O]

In some embodiments, L is

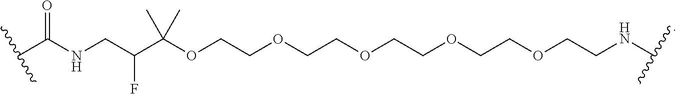 [SMILES: *NCCOCCOCCOCCOCCOC(C)(C)C(F)CNC(*)=O]

In some embodiments, L is

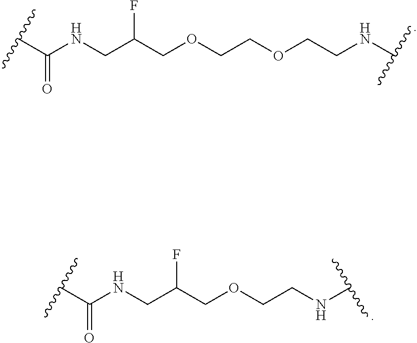 [SMILES: *NCCOCCOCC(F)CNC(*)=O ; *NCCOCC(F)CNC(*)=O]

In some embodiments, L is

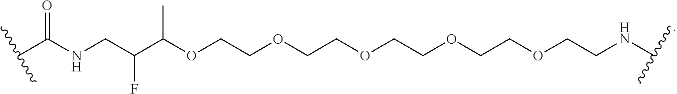 [SMILES: *NCCOCCOCCOCCOCCOC(C)C(F)CNC(*)=O]

In some embodiments, L is

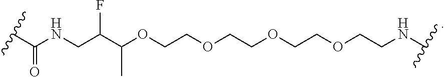 [SMILES: *NCCOCCOCCOCCOC(C)C(F)CNC(*)=O]

In some embodiments, L is

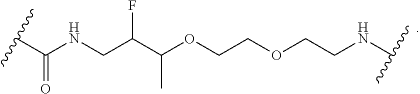 [SMILES: *NCCOCCOC(C)C(F)CNC(*)=O]

In some embodiments, L is

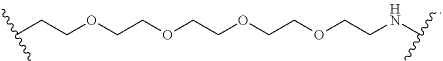 [SMILES: *CCOCCOCCOCCOCCN*]

In some embodiments, L is

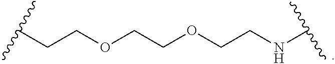 [SMILES: *CCOCCOCCN*]

In some embodiments, L is

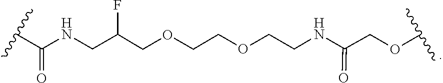 [SMILES: *OCC(=O)NCCOCCOCC(F)CNC(*)=O]

In some embodiments, L is

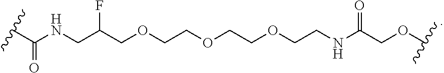 [SMILES: *OCC(=O)NCCOCCOCCOCC(F)CNC(*)=O]

In some embodiments, L is

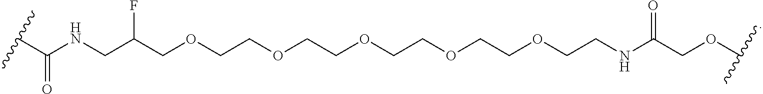 [SMILES: *OCC(=O)NCCOCCOCCOCCOCCOCC(F)CNC(*)=O]

In some embodiments, L is

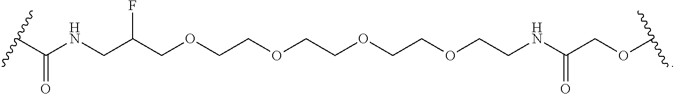 [SMILES: *OCC(=O)NCCOCCOCCOCCOCC(F)CNC(*)=O]

In some embodiments, L is

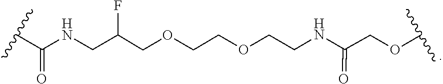 [SMILES: *OCC(=O)NCCOCCOCC(F)CNC(*)=O]

In some embodiments, L is

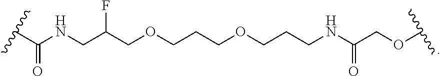 [SMILES: *OCC(=O)NCCCOCCCOCC(F)CNC(*)=O ; "."]

In some embodiments, L is

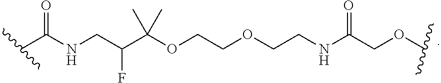 [SMILES: *OCC(=O)NCCOCCOC(C)(C)C(F)CNC(*)=O]

In some embodiments, L is

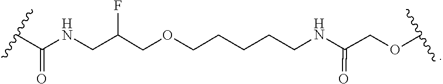 [SMILES: *OCC(=O)NCCCCCOCC(F)CNC(*)=O]

In some embodiments, L is

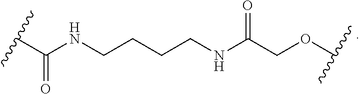 [SMILES: *OCC(=O)NCCCCNC(*)=O]

In some embodiments, L is

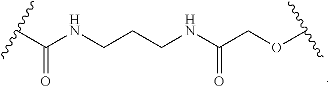 [SMILES: *OCC(=O)NCCCNC(*)=O]

In some embodiments, L is

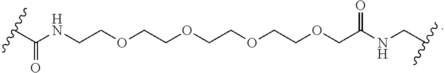 [SMILES: *CNC(=O)COCCOCCOCCOCCNC(*)=O]

In some embodiments, L is

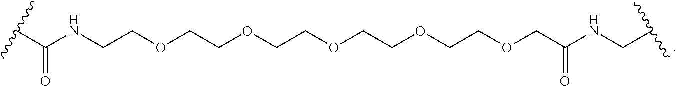 [SMILES: *CNC(=O)COCCOCCOCCOCCOCCNC(*)=O]

In some embodiments, L is

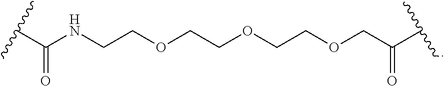 [SMILES: *C(=O)COCCOCCOCCNC(*)=O]

In some embodiments, L is

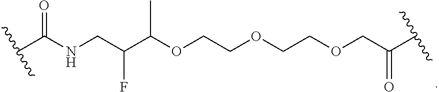 [SMILES: *C(=O)COCCOCCOC(C)C(F)CNC(*)=O]

In some embodiments, L is

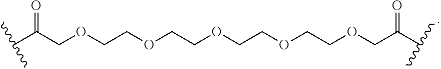 [SMILES: *C(=O)COCCOCCOCCOCCOCC(*)=O]

In some embodiments, L is

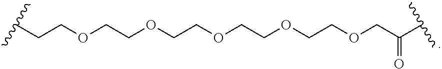 [SMILES: *CCOCCOCCOCCOCCOCC(*)=O]

In some embodiments, L is

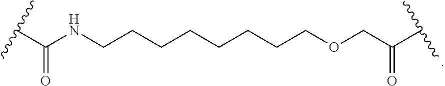 [SMILES: *C(=O)COCCCCCCCCNC(*)=O]

In some embodiments, L is

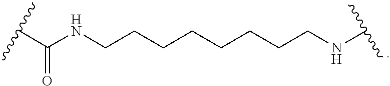 [SMILES: *NCCCCCCCCNC(*)=O]

In some embodiments, L is

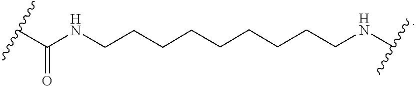 [SMILES: *NCCCCCCCCCNC(*)=O]

In some embodiments, L is

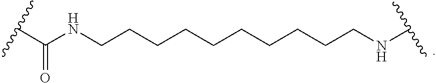 [SMILES: *NCCCCCCCCCCNC(*)=O]

In some embodiments, L is

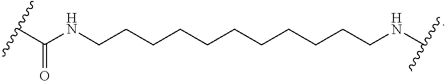 [SMILES: *NCCCCCCCCCCCNC(*)=O]

In some embodiments, L is

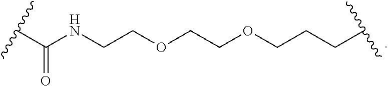 [SMILES: *CCCOCCOCCNC(*)=O]

In so embodiments, L is

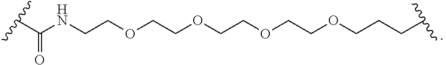 [SMILES: *CCCOCCOCCOCCOCCNC(*)=O]

In some embodiments, L is

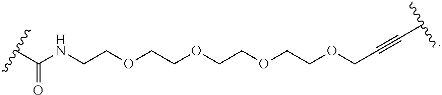 [SMILES: *C#CCOCCOCCOCCOCCNC(*)=O]

In some embodiments, L is

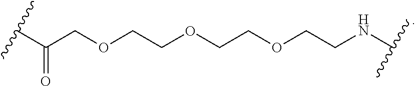 [SMILES: *NCCOCCOCCOCC(*)=O]

In some embodiments, L is

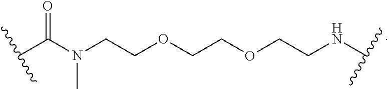 [SMILES: *NCCOCCOCCN(C)C(*)=O]

In some embodiments, L is

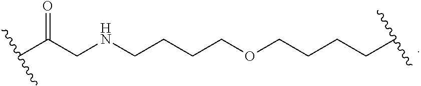 [SMILES: *CCCCOCCCCNCC(*)=O]

In some embodiments, L is

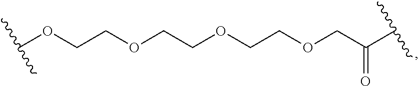 [SMILES: *OCCOCCOCCOCC(*)=O]

In some embodiments, L is

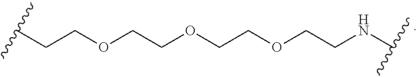 [SMILES: *CCOCCOCCOCCN*]

In some embodiments, L is

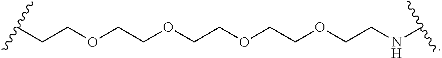 [SMILES: *CCOCCOCCOCCOCCN*]

In some embodiments, L is

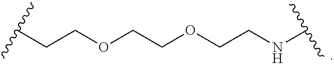 [SMILES: *CCOCCOCCN*]

In some embodiments, L is

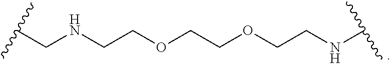 [SMILES: *CNCCOCCOCCN*]

In some embodiments, L is

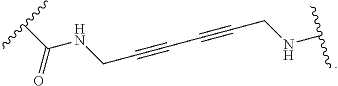 [SMILES: *NCC#CC#CCNC(*)=O ; "."]

In some embodiments, L is

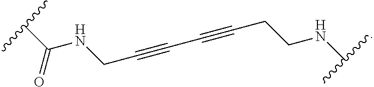 [SMILES: *NCCC#CC#CCNC(*)=O]

In some embodiments, L is

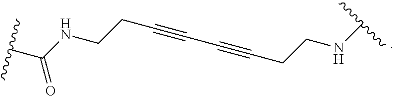 [SMILES: *NCCC#CC#CCCNC(*)=O ; "."]

In some embodiments, L is

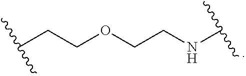 [SMILES: *CCOCCN*]

In some embodiments, L is

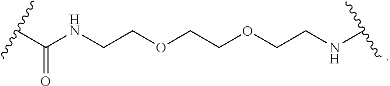 [SMILES: *NCCOCCOCCNC(*)=O ; "."]

In some embodiments, L is

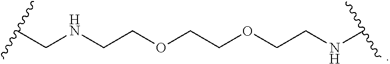 [SMILES: *CNCCOCCOCCN* ; "."]

In some embodiments, L is

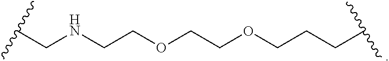 [SMILES: *CCCOCCOCCNC* ; "."]

In some embodiments, L is

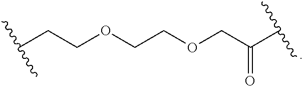 [SMILES: *CCOCCOCC(*)=O]

In some embodiments, L is

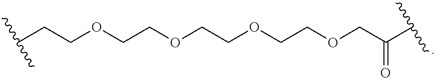 [SMILES: *CCOCCOCCOCCOCC(*)=O ; "."]

In some embodiments, L is

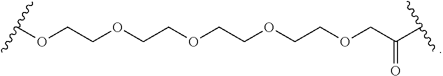 [SMILES: *OCCOCCOCCOCCOCC(*)=O ; "."]

In some embodiments, L is

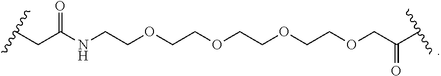 [SMILES: *CC(=O)NCCOCCOCCOCCOCC(*)=O ; "."]

In some embodiments, L is

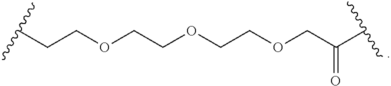 [SMILES: *CCOCCOCCOCC(*)=O]

In some embodiments, L is

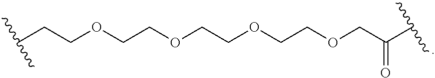 [SMILES: *CCOCCOCCOCCOCC(*)=O ; "."]

In some embodiments, L is

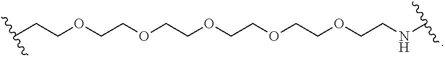 [SMILES: *CCOCCOCCOCCOCCOCCN* ; "."]

In some embodiments, L is

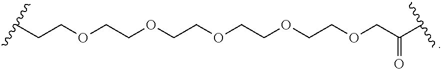 [SMILES: *CCOCCOCCOCCOCCOCC(*)=O]

In some embodiments, L is

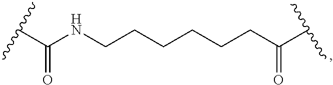 [SMILES: *C(=O)CCCCCCNC(*)=O ; ","]

In some embodiments, L is

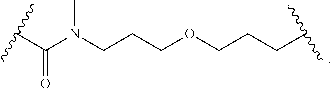 [SMILES: *CCCOCCCN(C)C(*)=O ; "."]

In some embodiments, L is

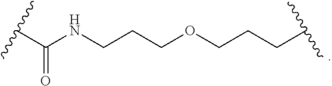 [SMILES: *CCCOCCCNC(*)=O ; "."]

In some embodiments, L is

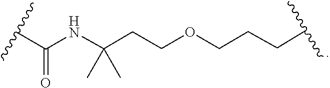 [SMILES: *CCCOCCC(C)(C)NC(*)=O]

In some embodiments, L is

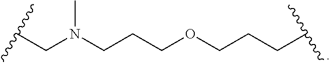 [SMILES: *CCCOCCCN(C)C*]

In some embodiments, L is

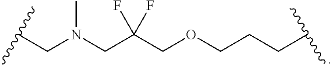 [SMILES: *CCCOCC(F)(F)CN(C)C*]

In some embodiments, L is

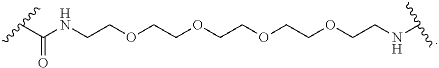 [SMILES: *NCCOCCOCCOCCOCCNC(*)=O]

In some embodiments, L is

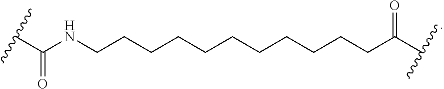 [SMILES: *C(=O)CCCCCCCCCCCNC(*)=O]

In some embodiments, L is

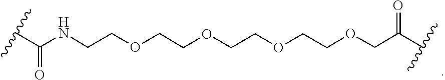 [SMILES: *C(=O)COCCOCCOCCOCCNC(*)=O]

In some embodiments, L is

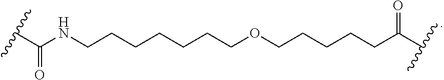 [SMILES: *C(=O)CCCCCOCCCCCCCNC(*)=O]

In some embodiments, L is

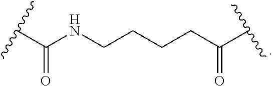 [SMILES: *C(=O)CCCCNC(*)=O]

In some embodiments, L is

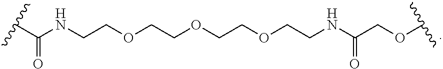 [SMILES: *OCC(=O)NCCOCCOCCOCCNC(*)=O]

In some embodiments, L is

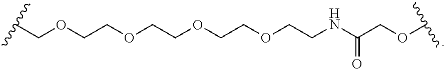 [SMILES: *COCCOCCOCCOCCNC(=O)CO*]

In some embodiments, L is

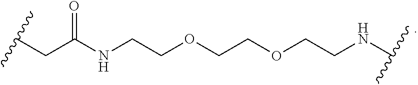 [SMILES: *CC(=O)NCCOCCOCCN*]

In some embodiments, L is

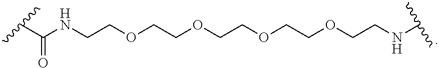 [SMILES: *NCCOCCOCCOCCOCCNC(*)=O]

In some embodiments, L is

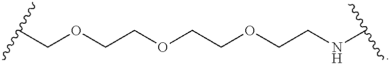 [SMILES: *COCCOCCOCCN*]

In some embodiments, L is

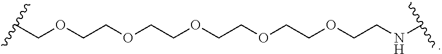 [SMILES: *COCCOCCOCCOCCOCCN*]

In some embodiments, L is

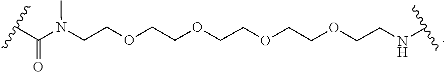 [SMILES: *NCCOCCOCCOCCOCCN(C)C(*)=O]

In some embodiments, L is

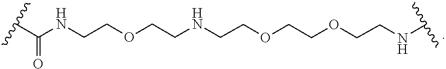 [SMILES: *NCCOCCOCCNCCOCCNC(*)=O]

In some embodiments, L is

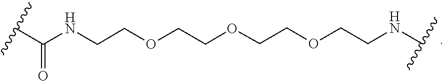 [SMILES: *NCCOCCOCCOCCNC(*)=O]

In some embodiments, L is

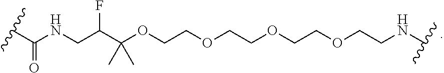 [SMILES: *NCCOCCOCCOCCOC(C)(C)C(F)CNC(*)=O]

In some embodiments, L is

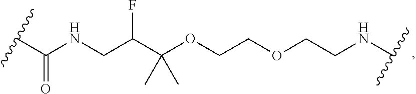 [SMILES: *NCCOCCOC(C)(C)C(F)CNC(*)=O]

In some embodiments, L is

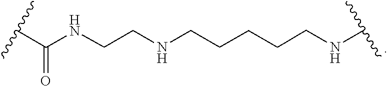 [SMILES: *NCCCCCNCCNC(*)=O]

In some embodiments, L is

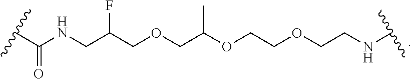 [SMILES: *NCCOCCOC(C)COCC(F)CNC(*)=O]

In some embodiments, L is

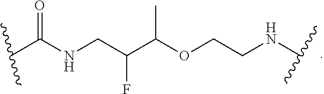 [SMILES: *NCCOC(C)C(F)CNC(*)=O]

In some embodiments, L is

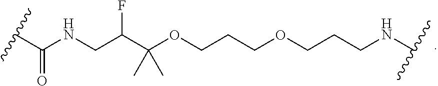 [SMILES: *NCCCOCCCOC(C)(C)C(F)CNC(*)=O]

In some embodiments, L is

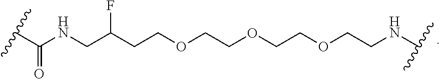 [SMILES: *NCCOCCOCCOCCC(F)CNC(*)=O]

In some embodiments, L is

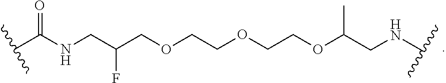 [SMILES: *NCC(C)OCCOCCOCC(F)CNC(*)=O]

In some embodiments, L is

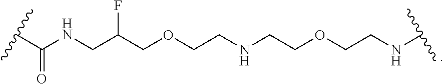 [SMILES: *NCCOCCNCCOCC(F)CNC(*)=O ; "."]

In some embodiments, L is

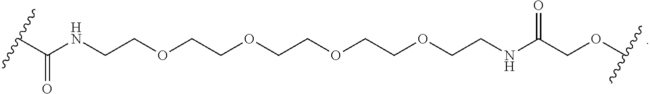 [SMILES: *OCC(=O)NCCOCCOCCOCCOCCNC(*)=O ; "."]

In some embodiments, L is

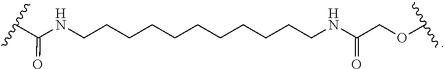 [SMILES: *OCC(=O)NCCCCCCCCCCCNC(*)=O]

In some embodiments, L is

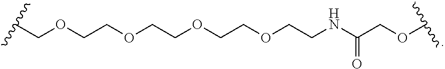 [SMILES: *COCCOCCOCCOCCNC(=O)CO*]

In some embodiments, L is

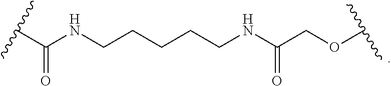 [SMILES: *OCC(=O)NCCCCCNC(*)=O]

In some embodiments, L is

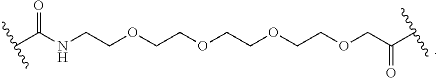 [SMILES: *C(=O)COCCOCCOCCOCCNC(*)=O]

In some embodiments, L is

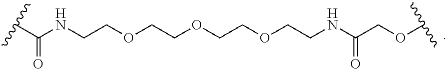 [SMILES: *OCC(=O)NCCOCCOCCOCCNC(*)=O]

In some embodiments, L is

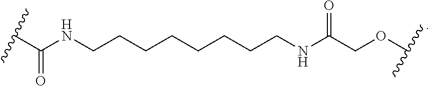 [SMILES: *OCC(=O)NCCCCCCCCNC(*)=O]

In some embodiments, L is

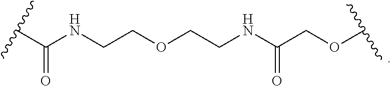 [SMILES: *OCC(=O)NCCOCCNC(*)=O]

In some embodiments, L is

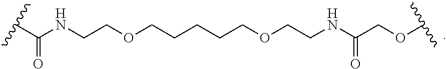 [SMILES: *OCC(=O)NCCOCCCCCOCCNC(*)=O]

In some embodiments, L is

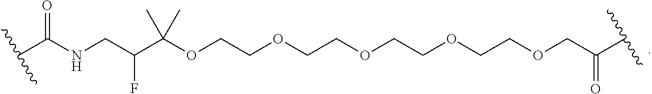 [SMILES: *C(=O)COCCOCCOCCOCCOC(C)(C)C(F)CNC(*)=O]

In some embodiments, L is

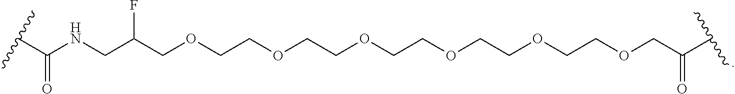 [SMILES: *C(=O)COCCOCCOCCOCCOCCOCC(F)CNC(*)=O]

In some embodiments, L is

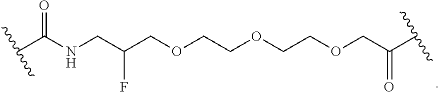 [SMILES: *C(=O)COCCOCCOCC(F)CNC(*)=O]

In some embodiments, L is

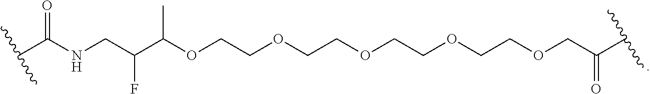 [SMILES: *C(=O)COCCOCCOCCOCCOC(C)C(F)CNC(*)=O]

In some embodiments, L is

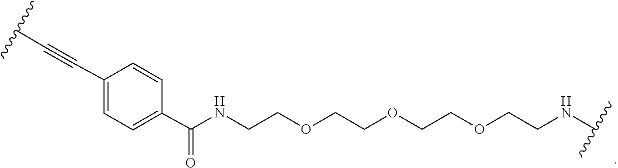 [SMILES: *C#Cc1ccc(C(=O)NCCOCCOCCOCCN*)cc1]

In some embodiments, L is

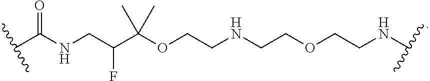 [SMILES: *NCCOCCNCCOC(C)(C)C(F)CNC(*)=O]

In some embodiments, L is

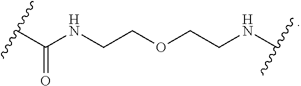 [SMILES: *NCCOCCNC(*)=O]

In some embodiments, L is

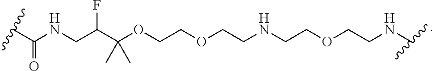 [SMILES: *NCCOCCNCCOCCOC(C)(C)C(F)CNC(*)=O]

In some embodiments, L is

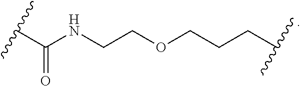 [SMILES: *CCCOCCNC(*)=O]

In some embodiments, L is

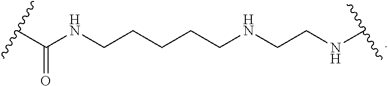 [SMILES: *NCCNCCCCCNC(*)=O]

In some embodiments, L is

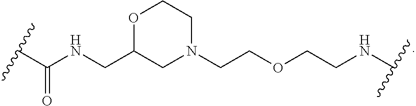 [SMILES: *NCCOCCN1CCOC(CNC(*)=O)C1]

In some embodiments, L is

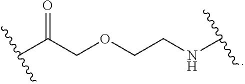 [SMILES: *NCCOCC(*)=O]

In some embodiments, L is

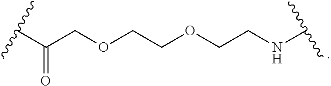 [SMILES: *NCCOCCOCC(*)=O ; "."]

In some embodiments, L is

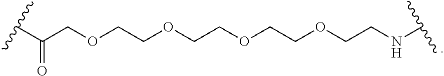 [SMILES: *NCCOCCOCCOCCOCC(*)=O ; "."]

In some embodiments, L is

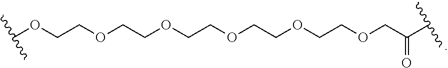 [SMILES: *OCCOCCOCCOCCOCCOCC(*)=O]

In some embodiments, L is

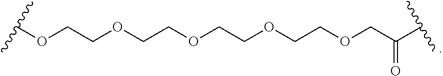 [SMILES: *OCCOCCOCCOCCOCC(*)=O ; "."]

In some embodiments, L is

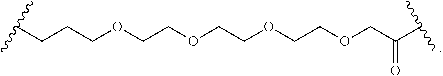 [SMILES: *CCCOCCOCCOCCOCC(*)=O ; "."]

In some embodiments, L is

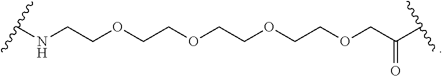 [SMILES: *NCCOCCOCCOCCOCC(*)=O ; "."]

In some embodiments, L is

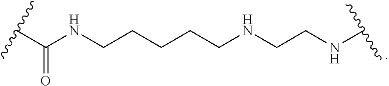 [SMILES: *NCCNCCCCCNC(*)=O]

In some embodiments, L is

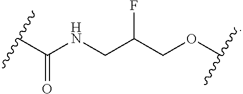 [SMILES: *OCC(F)CNC(*)=O]

In some embodiments, L is

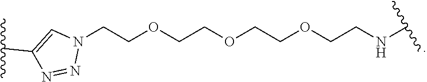 [SMILES: *NCCOCCOCCOCCn1cc(*)nn1]

In some embodiments, L is

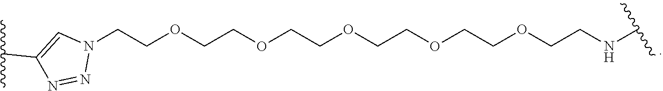 [SMILES: *NCCOCCOCCOCCOCCOCCn1cc(*)nn1]

In some embodiments, L is

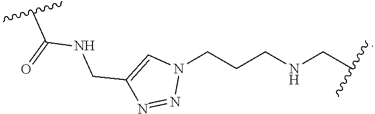 [SMILES: *CNCCCn1cc(CNC(*)=O)nn1]

In some embodiments, L is

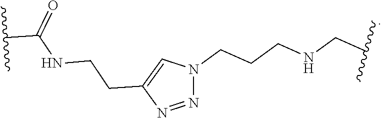 [SMILES: *CNCCCn1cc(CCNC(*)=O)nn1]

In some embodiments, L is

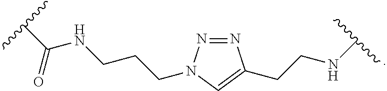 [SMILES: *NCCc1cn(CCCNC(*)=O)nn1]

In some embodiments, L is

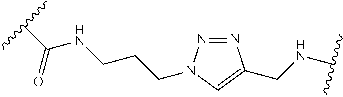 [SMILES: *NCc1cn(CCCNC(*)=O)nn1]

In some embodiments, L is

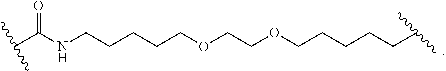 [SMILES: *CCCCCOCCOCCCCCNC(*)=O]

In some embodiments, L is

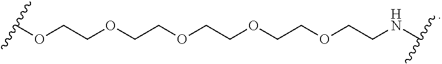 [SMILES: *NCCOCCOCCOCCOCCO*]

In some embodiments, L is

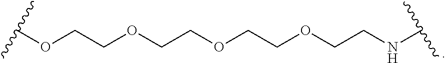 [SMILES: *NCCOCCOCCOCCO*]

In some embodiments, L is

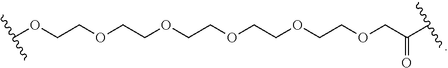 [SMILES: *OCCOCCOCCOCCOCCOCC(*)=O]

In some embodiments, L is

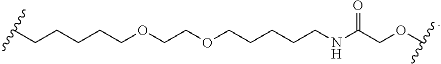 [SMILES: *CCCCCOCCOCCCCCNC(=O)CO*]

In some embodiments, L is

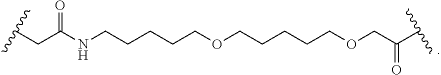 [SMILES: *CC(=O)NCCCCCOCCCCCOCC(*)=O ; "."]

In some embodiments, L is

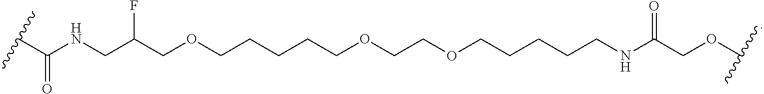 [SMILES: *OCC(=O)NCCCCCOCCOCCCCCOCC(F)CNC(*)=O]

In some embodiments, L is

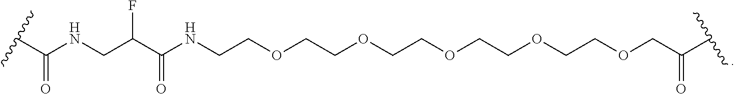 [SMILES: *C(=O)COCCOCCOCCOCCOCCNC(=O)C(F)CNC(*)=O]

In some embodiments, L is

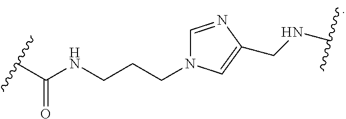 [SMILES: *NCc1cn(CCCNC(*)=O)cn1]

In some embodiments, L is

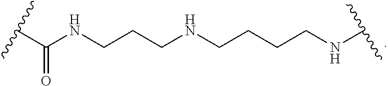 [SMILES: *NCCCCNCCCNC(*)=O]

In some embodiments, L is

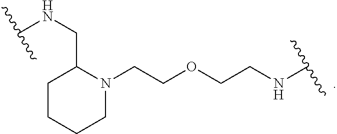 [SMILES: *NCCOCCN1CCCCC1CN*]

In some embodiments, L is

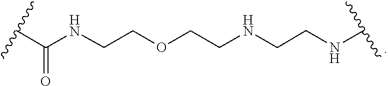 [SMILES: *NCCNCCOCCNC(*)=O]

In some embodiments, L is

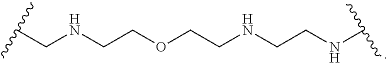 [SMILES: *CNCCOCCNCCN*]

In some embodiments, L is

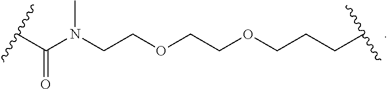 [SMILES: *CCCOCCOCCN(C)C(*)=O]

In some embodiments, L is

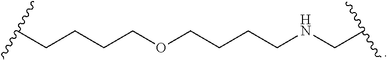 [SMILES: *CCCCOCCCCNC*]

In some embodiments, L is

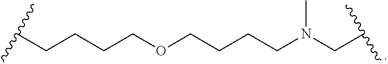 [SMILES: *CCCCOCCCCN(C)C*]

In some embodiments, L is

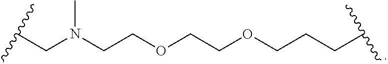 [SMILES: *CCCOCCOCCN(C)C*]

In some embodiments, L is

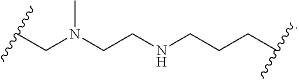 [SMILES: *CCCNCCN(C)C*]

In some embodiments, L is

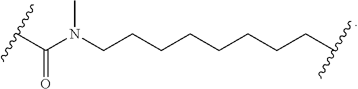 [SMILES: *CCCCCCCCN(C)C(*)=O]

In some embodiments, L is

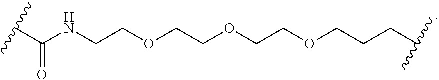 [SMILES: *CCCOCCOCCOCCNC(*)=O]

In some embodiments, L is

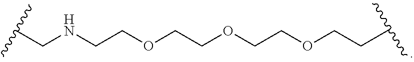 [SMILES: *CCOCCOCCOCCNC*]

In some embodiments, L is

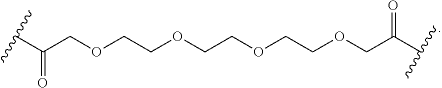 [SMILES: *C(=O)COCCOCCOCCOCC(*)=O]

In some embodiment L is

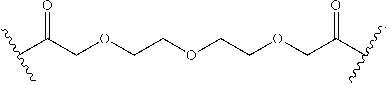 [SMILES: *C(=O)COCCOCCOCC(*)=O]

In some embodiments, L is

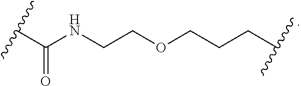 [SMILES: *CCCOCCNC(*)=O]

In some embodiments, L is

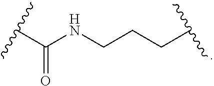 [SMILES: *CCCNC(*)=O]

In some embodiments, L is

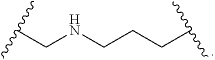 [SMILES: *CCCNC*]

In some embodiments, L is

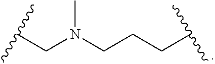 [SMILES: *CCCN(C)C*]

In some embodiments, L is

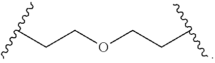 [SMILES: *CCOCC*]

In some embodiments, L is

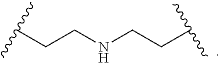 [SMILES: *CCNCC*]

In some embodiments, L is

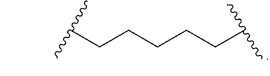

In some embodiments, L is

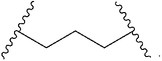 [SMILES: *CCC*]

In some embodiments L in some embodiments, L is

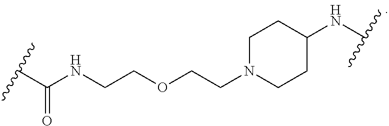 [SMILES: *NC1CCN(CCOCCNC(*)=O)CC1]

In some embodiments, L is

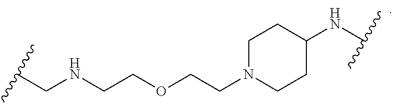 [SMILES: *CNCCOCCN1CCC(N*)CC1]

In some embodiments, L is

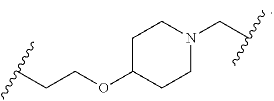 [SMILES: *CCOC1CCN(C*)CC1]

In some embodiments, L is

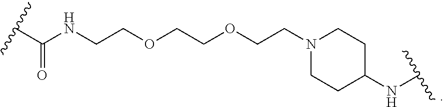 [SMILES: *NC1CCN(CCOCCOCCNC(*)=O)CC1]

In some embodiments, L is

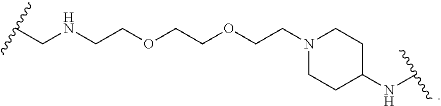 [SMILES: *CNCCOCCOCCN1CCC(N*)CC1]

In some embodiments, L is

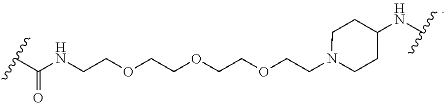 [SMILES: *NC1CCN(CCOCCOCCOCCNC(*)=O)CC1]

In some embodiments, L is

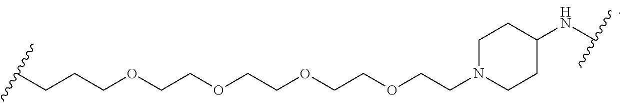 [SMILES: *CCCOCCOCCOCCOCCN1CCC(N*)CC1]

In some embodiments, L is

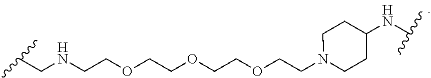 [SMILES: *CNCCOCCOCCOCCN1CCC(N*)CC1]

In some embodiments, L is

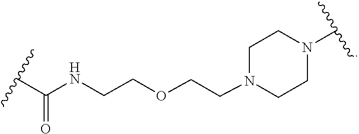 [SMILES: *C(=O)NCCOCCN1CCN(*)CC1]

In some embodiments, L is

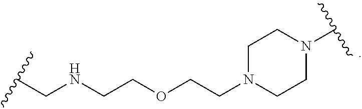 [SMILES: *CNCCOCCN1CCN(*)CC1]

In some embodiments, L is

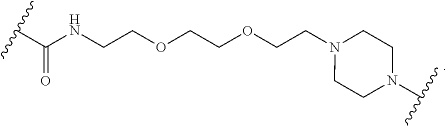 [SMILES: *C(=O)NCCOCCOCCN1CCN(*)CC1]

In some embodiments, L is

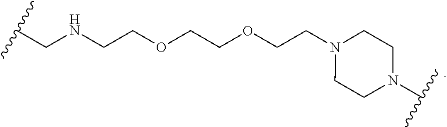 [SMILES: *CNCCOCCOCCN1CCN(*)CC1]

In some embodiments, L is

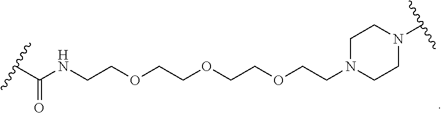 [SMILES: *C(=O)NCCOCCOCCOCCN1CCN(*)CC1]

In some embodiments, L is

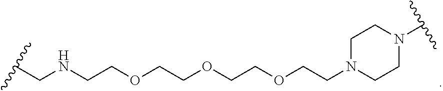 [SMILES: *CNCCOCCOCCOCCN1CCN(*)CC1]

In some embodiments, L is

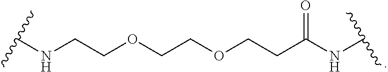 [SMILES: *NCCOCCOCCC(=O)N*]

In some embodiments, L is

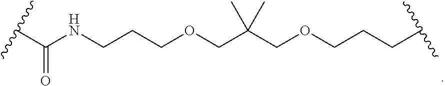 [SMILES: *CCCOCC(C)(C)COCCCNC(*)=O]

In some embodiments, L is

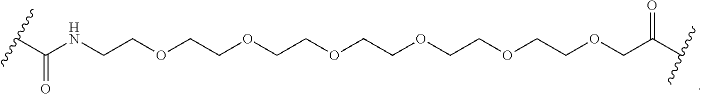 [SMILES: *C(=O)COCCOCCOCCOCCOCCOCCNC(*)=O]

In some embodiments, L is

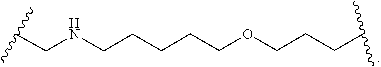 [SMILES: *CCCOCCCCCNC*]

In some embodiments, L is

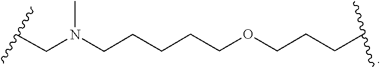 [SMILES: *CCCOCCCCCN(C)C*]

In some embodiments, L is

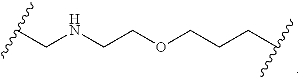 [SMILES: *CCCOCCNC*]

In some embodiments, L is

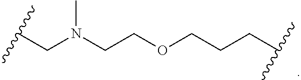 [SMILES: *CCCOCCN(C)C*]

In some embodiments, L is

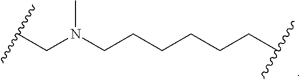 [SMILES: *CCCCCCN(C)C*]

In some embodiments, L is

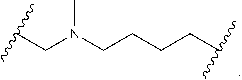 [SMILES: *CCCCN(C)C*]

In some embodiments, L is

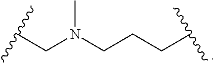 [SMILES: *CCCN(C)C*]

In some embodiments, L is

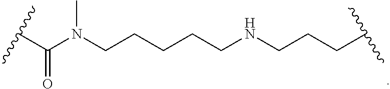 [SMILES: *CCCNCCCCCN(C)C(*)=O]

In some embodiments, L is

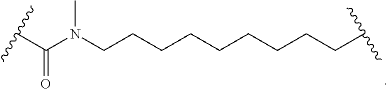 [SMILES: *CCCCCCCCCN(C)C(*)=O]

In some embodiments, L is

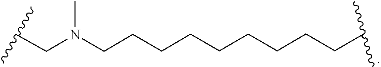 [SMILES: *CCCCCCCCCN(C)C*]

In some embodiments, L is

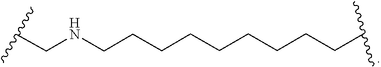 [SMILES: *CCCCCCCCCNC*]

In some embodiments, L is

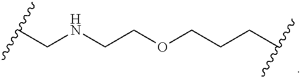 [SMILES: *CCCOCCNC*]

In some embodiments, L is

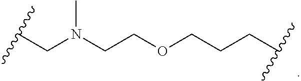 [SMILES: *CCCOCCN(C)C*]

In some embodiments, L is

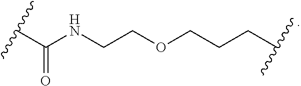 [SMILES: *CCCOCCNC(*)=O]

In some embodiments, L is

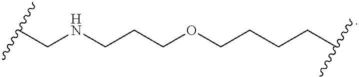 [SMILES: *CCCCOCCCNC*]

In some embodiments, L is

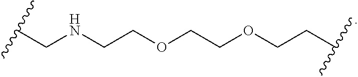 [SMILES: *CCOCCOCCNC*]

In some embodiments, L is

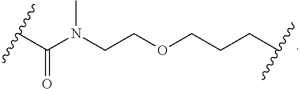 [SMILES: *CCCOCCN(C)C(*)=O]

In some embodiments, L is

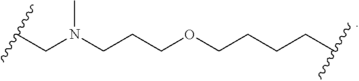 [SMILES: *CCCCOCCCN(C)C*]

In some embodiments, L is

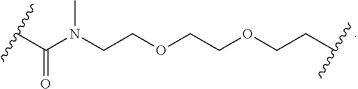 [SMILES: *CCOCCOCCN(C)C(*)=O]

In some embodiments, L is

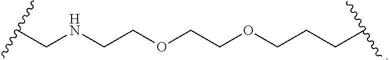 [SMILES: *CCCOCCOCCNC* ; "."]

In some embodiments, L is

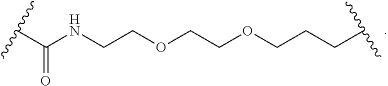 [SMILES: *CCCOCCOCCNC(*)=O]

In some embodiments, L is

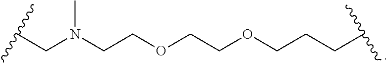 [SMILES: *CCCOCCOCCN(C)C*]

In some embodiments, L is

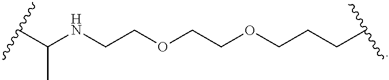 [SMILES: *CCCOCCOCCNC(*)C]

In some embodiments, L is

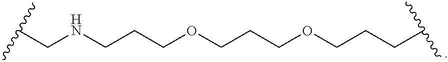 [SMILES: *CCCOCCCOCCCNC*]

In some embodiments, L is

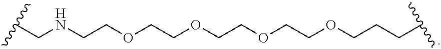 [SMILES: *CCCOCCOCCOCCOCCNC*]

In some embodiments, L is In some embodiments, L is

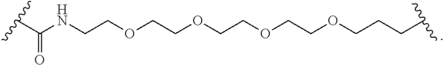 [SMILES: *CCCOCCOCCOCCOCCNC(*)=O]

In some embodiments, L is

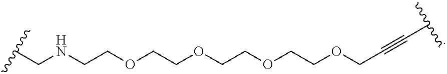 [SMILES: *C#CCOCCOCCOCCOCCNC*]

In some embodiments, L is

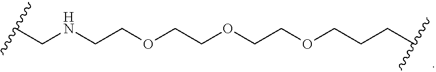 [SMILES: *CCCOCCOCCOCCNC*]

In some embodiments, L is

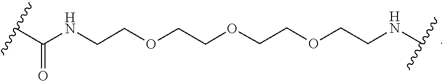 [SMILES: *NCCOCCOCCOCCNC(*)=O]

In some embodiments, L is

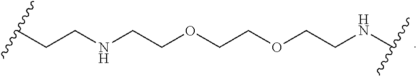 [SMILES: *CCNCCOCCOCCN*]

In some embodiments, L is

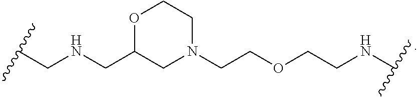 [SMILES: *CNCC1CN(CCOCCN*)CCO1]

In some embodiments, L is

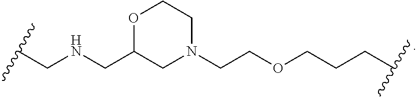 [SMILES: *CCCOCCN1CCOC(CNC*)C1]

In some embodiments, L is

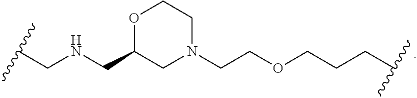 [SMILES: *CCCOCCN1CCO[C@H](CNC*)C1]

In some embodiments, L is

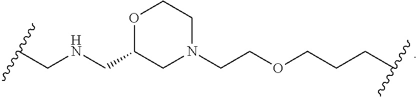 [SMILES: *CCCOCCN1CCO[C@@H](CNC*)C1]

In some embodiments, L is

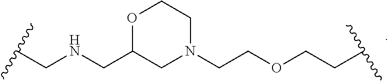 [SMILES: *CCOCCN1CCOC(CNC*)C1]

In some embodiments, L is

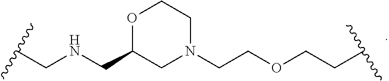 [SMILES: *CCOCCN1CCO[C@H](CNC*)C1]

In some embodiments, L is

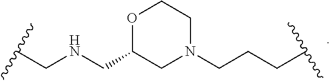 [SMILES: *CCCN1CCO[C@@H](CNC*)C1]

In some embodiments, L is

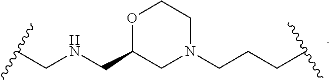 [SMILES: *CCCN1CCO[C@H](CNC*)C1]

In some embodiments, L is

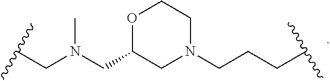 [SMILES: *CCCN1CCO[C@@H](CN(C)C*)C1]

In some embodiments, L is

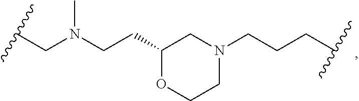 [SMILES: *CCCN1CCO[C@H](CCN(C)C*)C1]

In some embodiments, L is

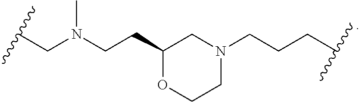 [SMILES: *CCCN1CCO[C@@H](CCN(C)C*)C1]

In some embodiments L is

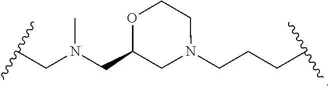 [SMILES: *CCCN1CCO[C@H](CN(C)C*)C1]

In some embodiments L is

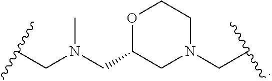 [SMILES: *CN(C)C[C@H]1CN(C*)CCO1]

In some embodiments, L is

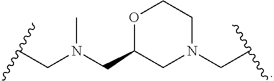 [SMILES: *CN(C)C[C@@H]1CN(C*)CCO1]

In some embodiments, L is

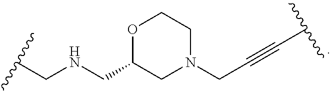 [SMILES: *C#CCN1CCO[C@@H](CNC*)C1]

In some embodiments, L is

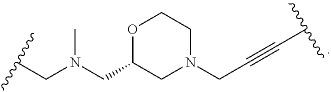 [SMILES: *C#CCN1CCO[C@@H](CN(C)C*)C1]

In some embodiments, L is

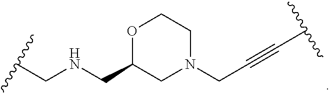 [SMILES: *C#CCN1CCO[C@H](CNC*)C1]

In some embodiments, L is

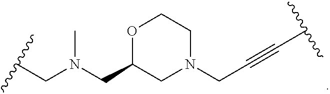 [SMILES: *C#CCN1CCO[C@H](CN(C)C*)C1]

In some embodiments, L is

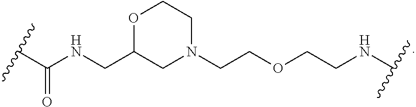 [SMILES: *NCCOCCN1CCOC(CNC(*)=O)C1]

In some embodiments, L is

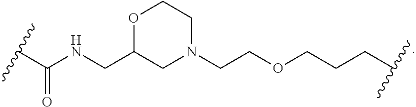 [SMILES: *CCCOCCN1CCOC(CNC(*)=O)C1]

In some embodiments, L is

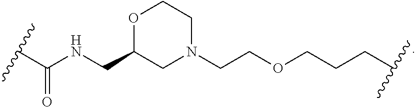 [SMILES: *CCCOCCN1CCO[C@H](CNC(*)=O)C1]

In some embodiments, L is

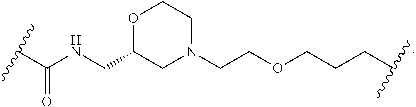 [SMILES: *CCCOCCN1CCO[C@@H](CNC(*)=O)C1]

In some embodiments, L is

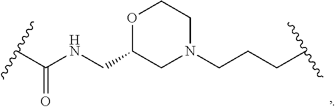 [SMILES: *CCCN1CCO[C@@H](CNC(*)=O)C1]

In some embodiments, L is

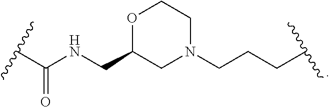 [SMILES: *CCCN1CCO[C@H](CNC(*)=O)C1]

In some embodiments, L is

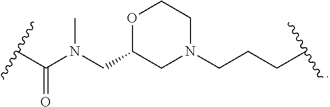 [SMILES: *CCCN1CCO[C@@H](CN(C)C(*)=O)C1]

In some embodiments, L is

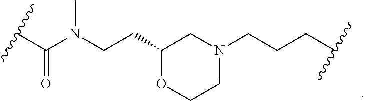 [SMILES: *CCCN1CCO[C@H](CCN(C)C(*)=O)C1]

In some embodiments, L is

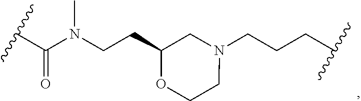 [SMILES: *CCCN1CCO[C@@H](CCN(C)C(*)=O)C1]

In some embodiments, L is

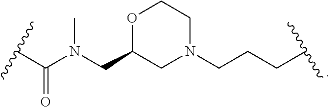 [SMILES: *CCCN1CCO[C@H](CN(C)C(*)=O)C1]

In some embodiments, L is

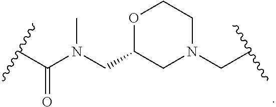 [SMILES: *CN1CCO[C@@H](CN(C)C(*)=O)C1]

In some embodiments, L is

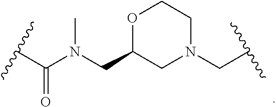 [SMILES: *CN1CCO[C@H](CN(C)C(*)=O)C1]

In some embodiments, L is

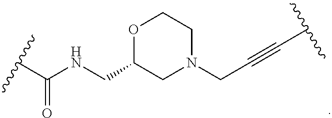 [SMILES: *C#CCN1CCO[C@@H](CNC(*)=O)C1]

In some embodiments, L is

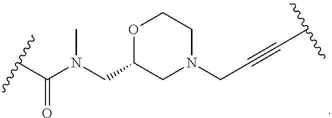 [SMILES: *C#CCN1CCO[C@@H](CN(C)C(*)=O)C1]

In some embodiments, L is

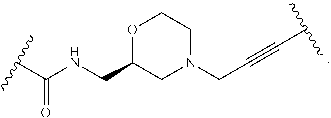 [SMILES: *C#CCN1CCO[C@H](CNC(*)=O)C1]

In some embodiments, L is

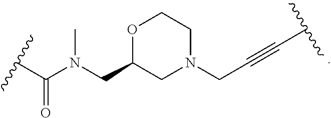 [SMILES: *C#CCN1CCO[C@H](CN(C)C(*)=O)C1]

In some embodiments, L is

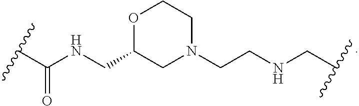 [SMILES: *CNCCN1CCO[C@@H](CNC(*)=O)C1]

In some embodiments, L is

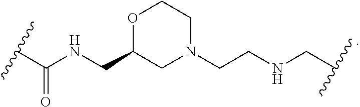 [SMILES: *CNCCN1CCO[C@H](CNC(*)=O)C1]

In some embodiments L is

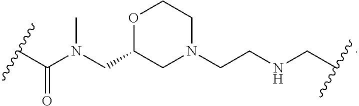 [SMILES: *CNCCN1CCO[C@@H](CN(C)C(*)=O)C1]

In some embodiments, L is

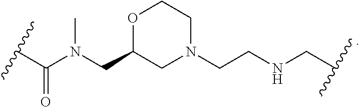 [SMILES: *CNCCN1CCO[C@H](CN(C)C(*)=O)C1]

In some embodiments, L is

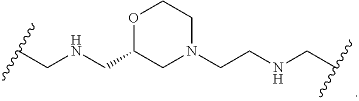 [SMILES: *CNCCN1CCO[C@@H](CNC*)C1]

In some embodiments, L is

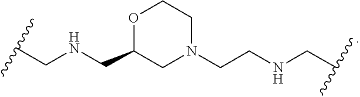 [SMILES: *CNCCN1CCO[C@H](CNC*)C1]

In some embodiments, L is

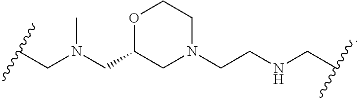 [SMILES: *CNCCN1CCO[C@@H](CN(C)C*)C1]

In some embodiments, L is

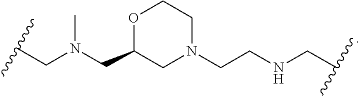 [SMILES: *CNCCN1CCO[C@H](CN(C)C*)C1]

In some embodiments, L is

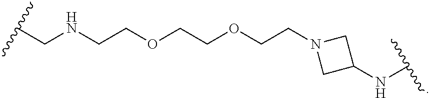 [SMILES: *CNCCOCCOCCN1CC(N*)C1]

In some embodiments, L is

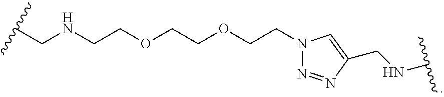 [SMILES: *CNCCOCCOCCn1cc(CN*)nn1]

In some embodiments, L is

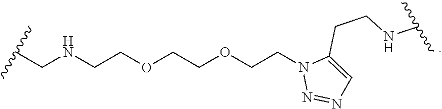 [SMILES: *CNCCOCCOCCn1nncc1CCN*]

In some embodiments, L is

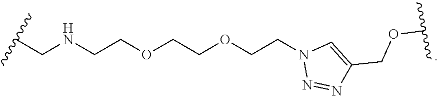 [SMILES: *CNCCOCCOCCn1cc(CO*)nn1]

In some embodiments, L is

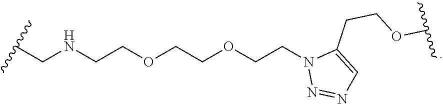 [SMILES: *CNCCOCCOCCn1nncc1CCO*]

In some embodiments, L is

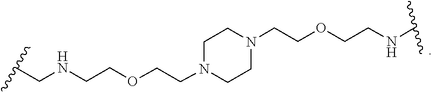 [SMILES: *CNCCOCCN1CCN(CCOCCN*)CC1]

In some embodiments, L is

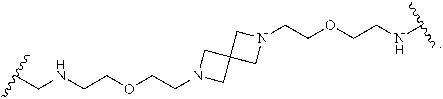 [SMILES: *CNCCOCCN1CC2(CN(CCOCCN*)C2)C1]

In some embodiments, L is

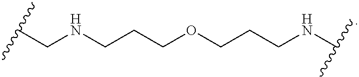 [SMILES: *CNCCCOCCCN*]

In some embodiments, L is

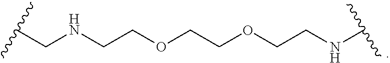 [SMILES: *CNCCOCCOCCN*]

In some embodiments, L is

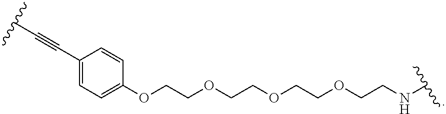 [SMILES: *C#Cc1ccc(OCCOCCOCCOCCN*)cc1]

In some embodiments, L is

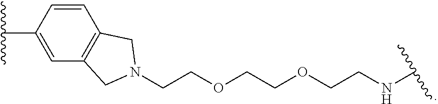 [SMILES: *NCCOCCOCCN1Cc2ccc(*)cc2C1]

In some embodiments, L is

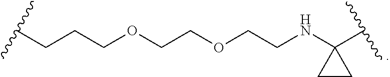 [SMILES: *CCCOCCOCCNC1(*)CC1]

In some embodiments, L is

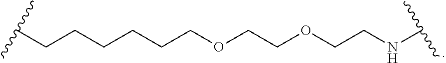 [SMILES: *CCCCCCOCCOCCN*]

In some embodiments, L is

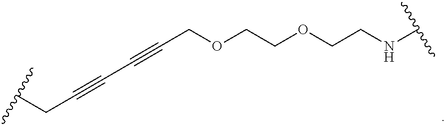 [SMILES: *CC#CC#CCOCCOCCN*]

In some embodiments, L is

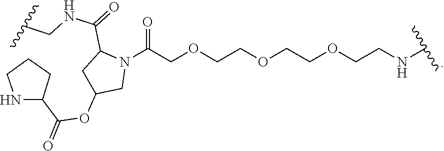 [SMILES: *CNC(=O)C1CC(OC(=O)C2CCCN2)CN1C(=O)COCCOCCOCCN*]

In some embodiments, L is

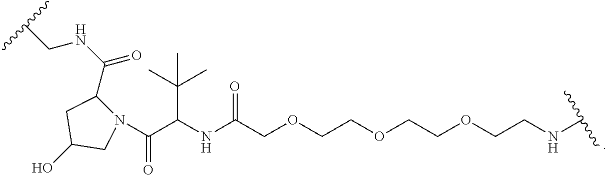 [SMILES: *CNC(=O)C1CC(O)CN1C(=O)C(NC(=O)COCCOCCOCCN*)C(C)(C)C ; "."]

In some embodiments, L is

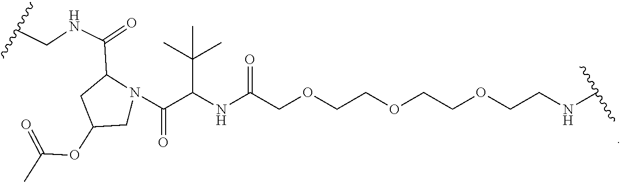 [SMILES: *CNC(=O)C1CC(OC(C)=O)CN1C(=O)C(NC(=O)COCCOCCOCCN*)C(C)(C)C]

In some embodiments, L is

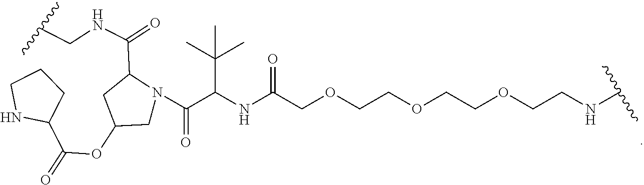 [SMILES: *CNC(=O)C1CC(OC(=O)C2CCCN2)CN1C(=O)C(NC(=O)COCCOCCOCCN*)C(C)(C)C]

In some embodiments, L is

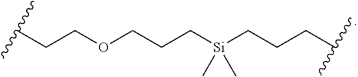 [SMILES: *CCC[Si](C)(C)CCCOCC*]

In some embodiments, L is

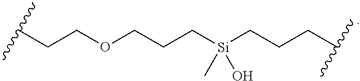 [SMILES: *CCC[Si](C)(O)CCCOCC*]

In some embodiments, L is

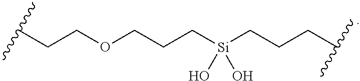 [SMILES: *CCC[Si](O)(O)CCCOCC*]

In some embodiments, L is

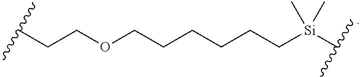 [SMILES: *CCOCCCCCC[Si](*)(C)C]

In some embodiments, L is

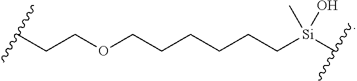 [SMILES: *CCOCCCCCC[Si](*)(C)O]

In some embodiments, L is

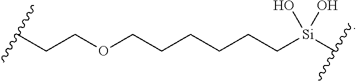 [SMILES: *CCOCCCCCC[Si](*)(O)O]

In some embodiments, L is

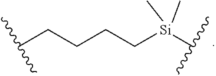 [SMILES: *CCCC[Si](*)(C)C]

In some embodiments, L is

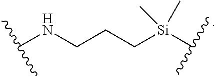 [SMILES: *NCCC[Si](*)(C)C]

In some embodiments, L is

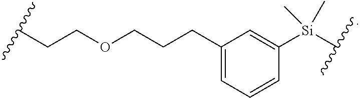 [SMILES: *CCOCCCc1cccc([Si](*)(C)C)c1]

In some embodiments, L is

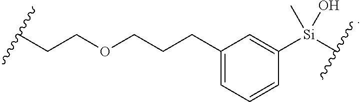 [SMILES: *CCOCCCc1cccc([Si](*)(C)O)c1]

In some embodiments, L is

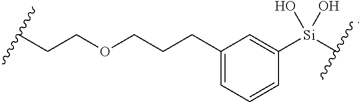 [SMILES: *CCOCCCc1cccc([Si](*)(O)O)c1]

In some embodiments, L is

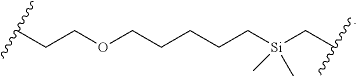 [SMILES: *CCOCCCCC[Si](C)(C)C*]

In some embodiments, L is

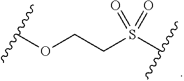 [SMILES: *OCCS(*)(=O)=O]

In some embodiments, L is

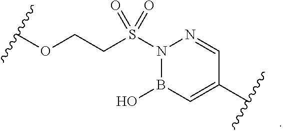 [SMILES: *OCCS(=O)(=O)N1N=CC(*)=CB1O]

In some embodiments, L is

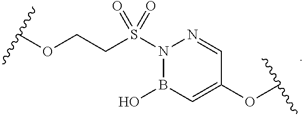 [SMILES: *OCCS(=O)(=O)N1N=CC(O*)=CB1O]

In some embodiments, L is

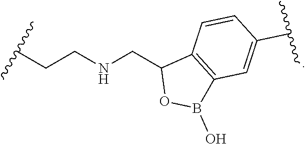 [SMILES: *CCNCC1OB(O)c2cc(*)ccc21]

In some embodiments, L is

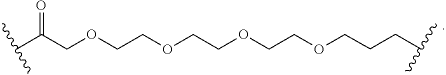 [SMILES: *CCCOCCOCCOCCOCC(*)=O]

In some embodiments, L is

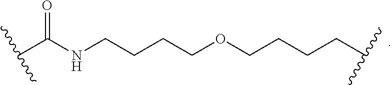 [SMILES: *CCCCOCCCCNC(*)=O]

In some embodiments, L is

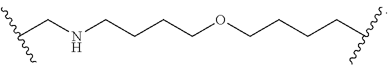 [SMILES: *CCCCOCCCCNC*]

In some embodiments, L is

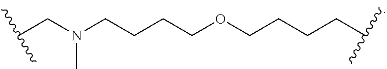 [SMILES: *CCCCOCCCCN(C)C*]

In some embodiments, L is

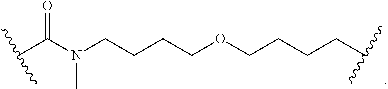 [SMILES: *CCCCOCCCCN(C)C(*)=O]

In some embodiments, L is

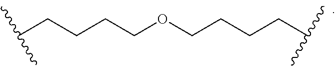 [SMILES: *CCCCOCCCC*]

In some embodiments, L is

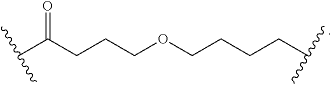 [SMILES: *CCCCOCCCC(*)=O]

In some embodiments, L is

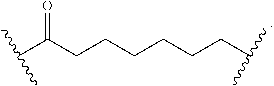 [SMILES: *CCCCCCC(*)=O ; "."]

In some embodiments, L is

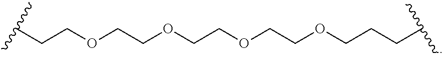 [SMILES: *CCCOCCOCCOCCOCC*]

In some embodiments, L is

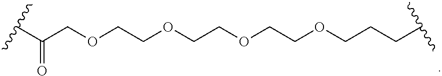 [SMILES: *CCCOCCOCCOCCOCC(*)=O]

In some embodiments, L is

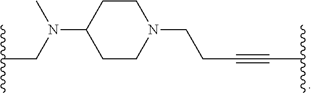 [SMILES: *C#CCCN1CCC(N(C)C*)CC1]

In some embodiments, L is

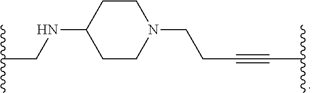 [SMILES: *C#CCCN1CCC(NC*)CC1]

In some embodiments, L is

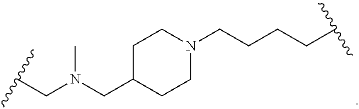 [SMILES: *CCCCN1CCC(CN(C)C*)CC1]

In some embodiments L is

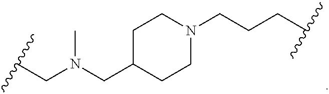 [SMILES: *CCCN1CCC(CN(C)C*)CC1]

In some embodiments, L is

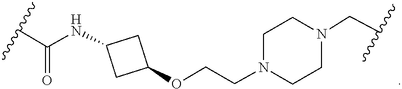 [SMILES: *CN1CCN(CCO[C@H]2C[C@H](NC(*)=O)C2)CC1]

In some embodiments, L is

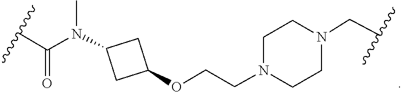 [SMILES: *CN1CCN(CCO[C@H]2C[C@H](N(C)C(*)=O)C2)CC1]

In some embodiments, L is

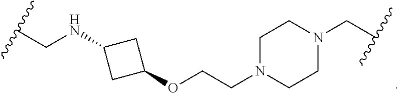 [SMILES: *CN[C@H]1C[C@H](OCCN2CCN(C*)CC2)C1]

In some embodiments, L is

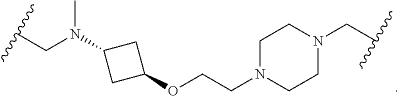 [SMILES: *CN1CCN(CCO[C@H]2C[C@H](N(C)C*)C2)CC1]

In some embodiments, L is

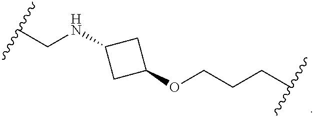 [SMILES: *CCCO[C@H]1C[C@H](NC*)C1]

In some embodiments, L is

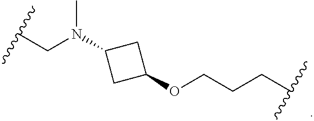 [SMILES: *CCCO[C@H]1C[C@H](N(C)C*)C1]

In some embodiments, L is

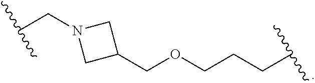 [SMILES: *CCCOCC1CN(C*)C1]

In some embodiments, L is

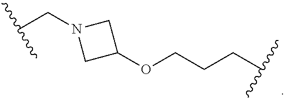 [SMILES: *CCCOC1CN(C*)C1]

In some embodiments, L is

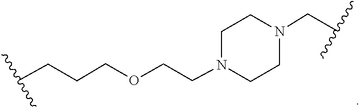 [SMILES: *CCCOCCN1CCN(C*)CC1]

In some embodiments, L is

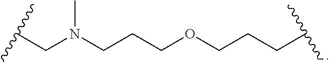 [SMILES: *CCCOCCCN(C)C*]

In some embodiments, L is

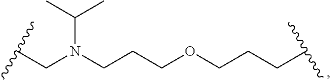 [SMILES: *CCCOCCCN(C*)C(C)C]

In some embodiments, L is

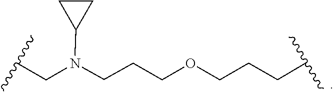 [SMILES: *CCCOCCCN(C*)C1CC1]

In some embodiments L is

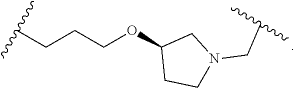 [SMILES: *CCCO[C@@H]1CCN(C*)C1]

In some embodiments, L is

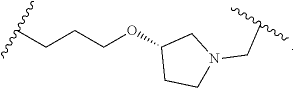 [SMILES: *CCCO[C@H]1CCN(C*)C1]

In some embodiments, L is

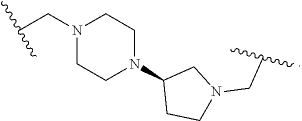 [SMILES: *CN1CCN([C@@H]2CCN(C*)C2)CC1]

In some embodiments, L is

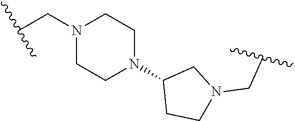 [SMILES: *CN1CCN([C@H]2CCN(C*)C2)CC1]

In some embodiments, L is

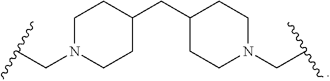 [SMILES: *CN1CCC(CC2CCN(C*)CC2)CC1]

In some embodiments, L is

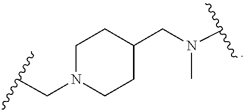 [SMILES: *CN1CCC(CN(*)C)CC1]

In some embodiments, L is

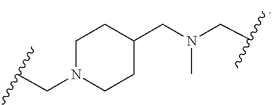 [SMILES: *CN(C)CC1CCN(C*)CC1]

In some embodiments, L is

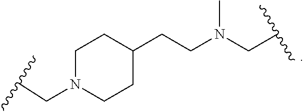 [SMILES: *CN(C)CCC1CCN(C*)CC1]

In some embodiments, L is

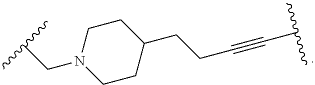 [SMILES: *C#CCCC1CCN(C*)CC1]

In some embodiments, L is

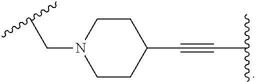 [SMILES: *C#CC1CCN(C*)CC1]

In some embodiments, L is

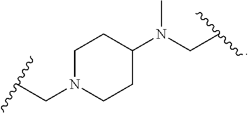 [SMILES: *CN1CCC(N(C)C*)CC1]

In some embodiments, L is

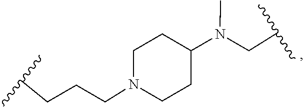 [SMILES: *CCCN1CCC(N(C)C*)CC1]

In some embodiments, L is

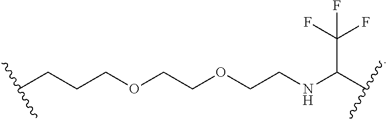 [SMILES: *CCCOCCOCCNC(*)C(F)(F)F]

In some embodiments L is

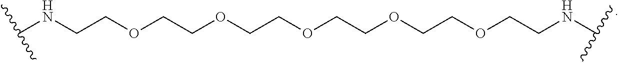 [SMILES: *NCCOCCOCCOCCOCCOCCN*]

In some embodiments, L is

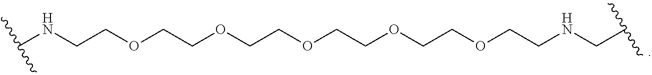 [SMILES: *CNCCOCCOCCOCCOCCOCCN*]

In some embodiments, L is

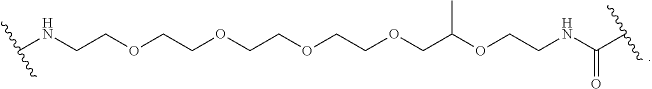 [SMILES: *NCCOCCOCCOCCOCC(C)OCCNC(*)=O]

In some embodiments, L is

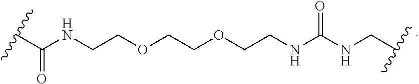 [SMILES: *CNC(=O)NCCOCCOCCNC(*)=O]

In some embodiments, L is

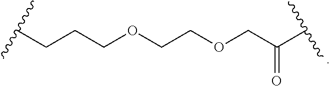 [SMILES: *CCCOCCOCC(*)=O]

In some embodiments, L is

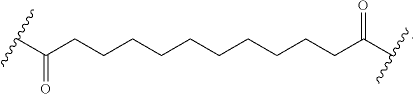 [SMILES: *C(=O)CCCCCCCCCCC(*)=O]

In some embodiments, L is

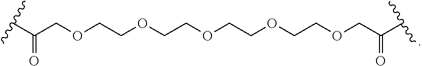 [SMILES: *C(=O)COCCOCCOCCOCCOCC(*)=O]

In some embodiments, L is

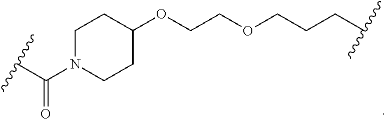 [SMILES: *CCCOCCOC1CCN(C(*)=O)CC1]

In some embodiments, L is

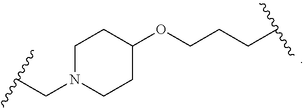 [SMILES: *CCCOC1CCN(C*)CC1]

In some embodiments, L is

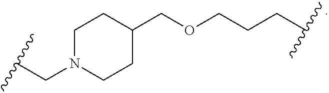 [SMILES: *CCCOCC1CCN(C*)CC1]

In some embodiments, L is

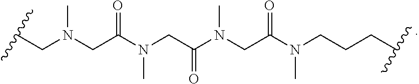 [SMILES: *CCCN(C)C(=O)CN(C)C(=O)CN(C)C(=O)CN(C)C*]

In some embodiments, L is

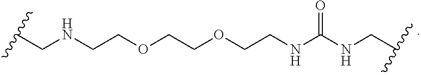 [SMILES: *CNCCOCCOCCNC(=O)NC*]

In some embodiments, L is

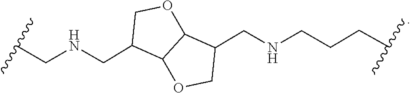 [SMILES: *CCCNCC1COC2C(CNC*)COC12]

In some embodiments, L is

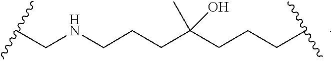 [SMILES: *CCCC(C)(O)CCCNC*]

In some embodiments, L is

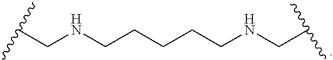 [SMILES: *CNCCCCCNC*]

In some embodiments, L is

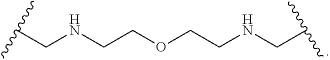 [SMILES: *CNCCOCCNC*]

In some embodiments, L is

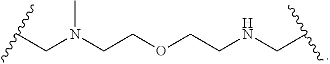 [SMILES: *CNCCOCCN(C)C*]

In some embodiments, L is

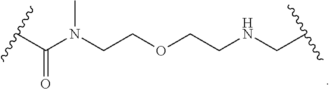 [SMILES: *CNCCOCCN(C)C(*)=O]

In some embodiments, L is

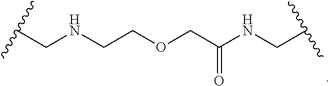 [SMILES: *CNCCOCC(=O)NC*]

In some embodiments, L is

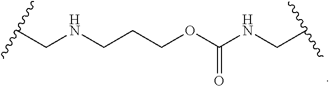 [SMILES: *CNCCCOC(=O)NC*]

In some embodiments, L is

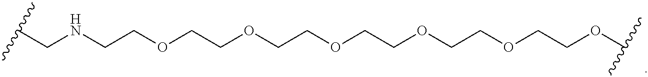 [SMILES: *CNCCOCCOCCOCCOCCOCCO*]

In some embodiments, L is

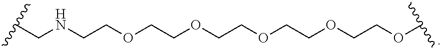 [SMILES: *CNCCOCCOCCOCCOCCO*]

In some embodiments, L is

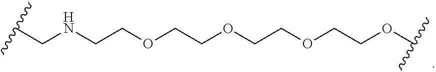 [SMILES: *CNCCOCCOCCOCCO*]

In some embodiments, L is

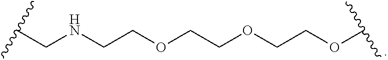 [SMILES: *CNCCOCCOCCO*]

In some embodiments, L is

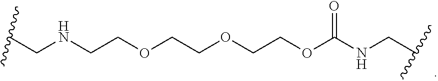 [SMILES: *CNCCOCCOCCOC(=O)NC*]

In some embodiments, L is

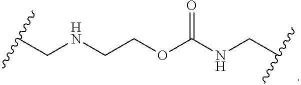 [SMILES: *CNCCOC(=O)NC*]

In some embodiments, L is

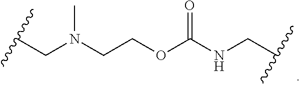 [SMILES: *CNC(=O)OCCN(C)C*]

In some embodiments, L is

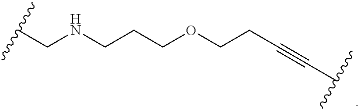 [SMILES: *C#CCCOCCCNC*]

In some embodiments, L is

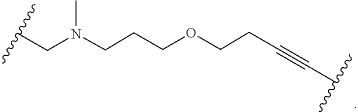 [SMILES: *C#CCCOCCCN(C)C*]

In some embodiments, L is

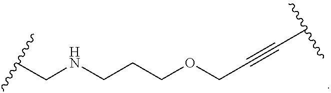 [SMILES: *C#CCOCCCNC*]

In some embodiments, L is

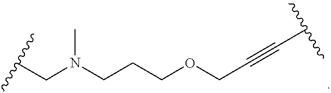 [SMILES: *C#CCOCCCN(C)C*]

In some embodiments, L is

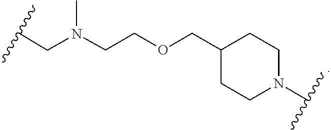 [SMILES: *CN(C)CCOCC1CCN(*)CC1]

In some embodiments, L is

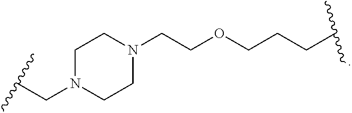 [SMILES: *CCCOCCN1CCN(C*)CC1]

In some embodiments, L is

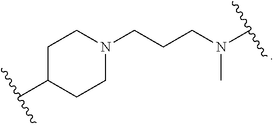 [SMILES: *C1CCN(CCCN(*)C)CC1]

In some embodiments, L is

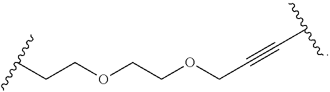 [SMILES: *C#CCOCCOCC*]

In some embodiments, L is

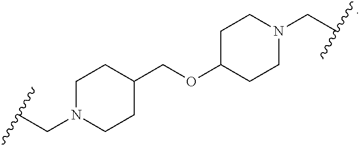 [SMILES: *CN1CCC(COC2CCN(C*)CC2)CC1]

In some embodiments L is

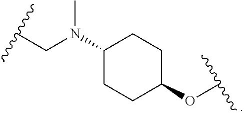 [SMILES: *CN(C)[C@H]1CC[C@H](O*)CC1]

In some embodiments, L is

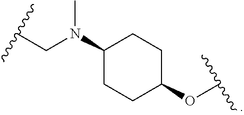 [SMILES: *CN(C)[C@H]1CC[C@@H](O*)CC1]

In some embodiments, L is

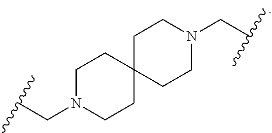 [SMILES: *CN1CCC2(CC1)CCN(C*)CC2]

In some embodiments, L is

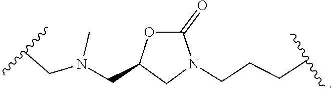 [SMILES: *CCCN1C[C@@H](CN(C)C*)OC1=O]

In some embodiments, L is

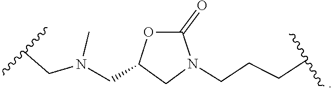 [SMILES: *CCCN1C[C@H](CN(C)C*)OC1=O]

In some embodiments, L is

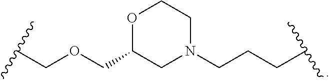 [SMILES: *CCCN1CCO[C@@H](COC*)C1]

In some embodiments, L is

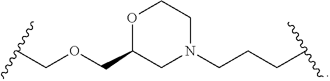 [SMILES: *CCCN1CCO[C@H](COC*)C1]

In some embodiments, L is

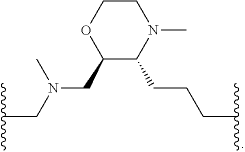 [SMILES: *CCC[C@@H]1[C@@H](CN(C)C*)OCCN1C]

In some embodiments, L is

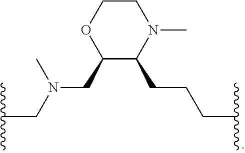 [SMILES: *CCC[C@H]1[C@@H](CN(C)C*)OCCN1C]

In some embodiments, L is

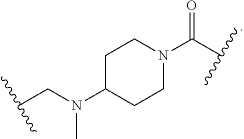 [SMILES: *CN(C)C1CCN(C(*)=O)CC1]

In some embodiments, L is

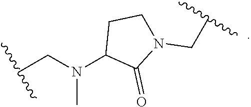 [SMILES: *CN1CCC(N(C)C*)C1=O]

In some embodiments, L is

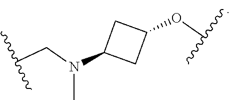 [SMILES: *CN(C)[C@H]1C[C@H](O*)C1]

In some embodiments, L is

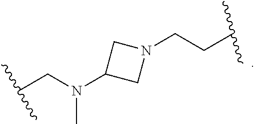 [SMILES: *CCN1CC(N(C)C*)C1]

In some embodiments, L is

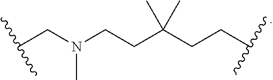 [SMILES: *CCC(C)(C)CCN(C)C*]

In some embodiments, L is

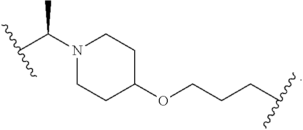 [SMILES: *CCCOC1CCN([C@@H](*)C)CC1]

In some embodiments, L is

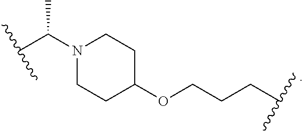 [SMILES: *CCCOC1CCN([C@H](*)C)CC1]

In some embodiments, L is

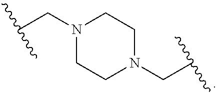 [SMILES: *CN1CCN(C*)CC1]

In some embodiments, L is

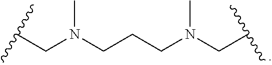 [SMILES: *CN(C)CCCN(C)C*]

In some embodiments, L is

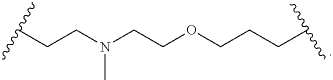 [SMILES: *CCCOCCN(C)CC*]

In some embodiments, L is

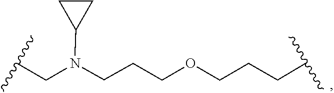 [SMILES: *CCCOCCCN(C*)C1CC1]

In some embodiments, L is

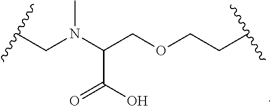 [SMILES: *CCOCC(C(=O)O)N(C)C*]

In some embodiments, L is

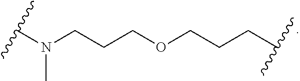 [SMILES: *CCCOCCCN(*)C]

In some embodiments, L is

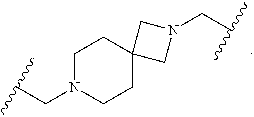 [SMILES: *CN1CCC2(CC1)CN(C*)C2]

In some embodiments, L is

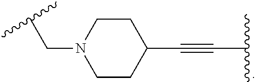 [SMILES: *C#CC1CCN(C*)CC1]

In some embodiments, L is

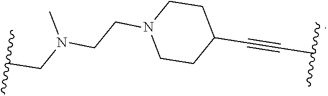 [SMILES: *C#CC1CCN(CCN(C)C*)CC1]

In some embodiments, L is

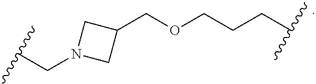 [SMILES: *CCCOCC1CN(C*)C1]

In some embodiments, L is

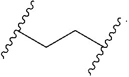 [SMILES: *CC*]

In some embodiments, L is

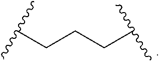 [SMILES: *CCC*]

In some embodiments, L is

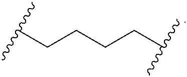 [SMILES: *CCCC*]

In some embodiments, L is

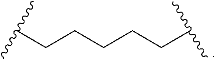 [SMILES: *CCCCC*]

In some embodiments, L is

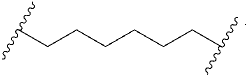 [SMILES: *CCCCCC*]

In some embodiments L is

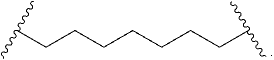 [SMILES: *CCCCCCC*]

In some embodiments, L is

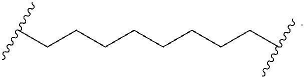 [SMILES: *CCCCCCCC*]

In some embodiments, L is

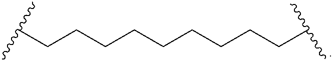 [SMILES: *CCCCCCCCC*]

In some embodiments, L is a covalent bond. In some embodiments, L is

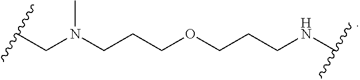 [SMILES: *CN(C)CCCOCCCN*]

In some embodiments, L is

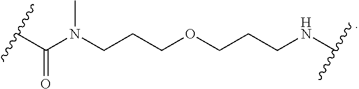 [SMILES: *NCCCOCCCN(C)C(*)=O]

In some embodiments, L is

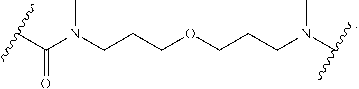 [SMILES: *C(=O)N(C)CCCOCCCN(*)C]

In some embodiments, L is

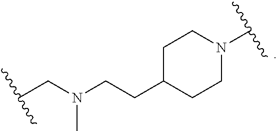 [SMILES: *CN(C)CCC1CCN(*)CC1]

In some embodiments L is

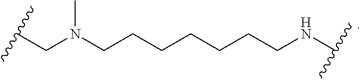 [SMILES: *CN(C)CCCCCCCN*]

In some embodiments, L is

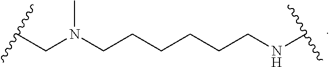 [SMILES: *CN(C)CCCCCCN*]

In some embodiments, L is

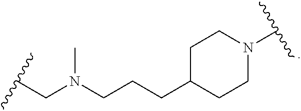 [SMILES: *CN(C)CCCC1CCN(*)CC1]

In some embodiments, L is

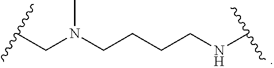 [SMILES: *CN(C)CCCCN*]

In some embodiments, L is

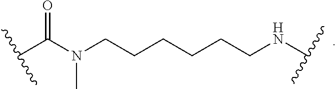 [SMILES: *NCCCCCCN(C)C(*)=O ; "."]

In some embodiments, L is

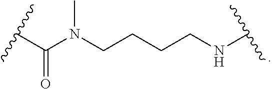 [SMILES: *NCCCCN(C)C(*)=O]

In some embodiments L is

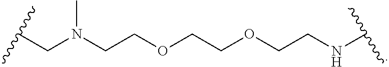 [SMILES: *CN(C)CCOCCOCCN*]

In some embodiments, L is

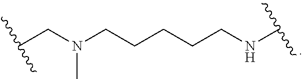 [SMILES: *CN(C)CCCCCN*]

In some embodiments, L is

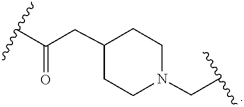 [SMILES: *CN1CCC(CC(*)=O)CC1]

In some embodiments, L is

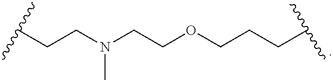 [SMILES: *CCCOCCN(C)CC*]

In some embodiments, L is N

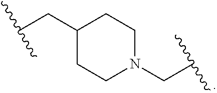 [SMILES: *CC1CCN(C*)CC1]

In some embodiments, L is

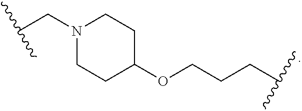 [SMILES: *CCCOC1CCN(C*)CC1]

In some embodiments, L is

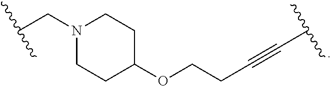 [SMILES: *C#CCCOC1CCN(C*)CC1]

In some embodiments, L is

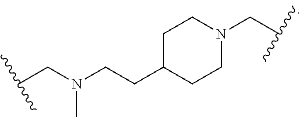 [SMILES: *CN(C)CCC1CCN(C*)CC1]

In some embodiments, L is

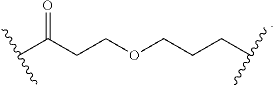 [SMILES: *CCCOCCC(*)=O ; "."]

In some embodiments, L is

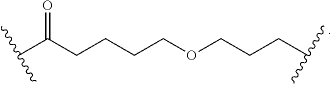 [SMILES: *CCCOCCCCC(*)=O]

In some embodiments, L is H

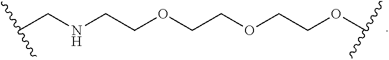 [SMILES: *CNCCOCCOCCO*]

In some embodiments, L is

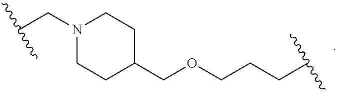 [SMILES: *CCCOCC1CCN(C*)CC1]

In some embodiments, L is

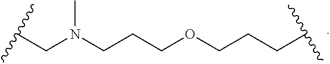 [SMILES: *CCCOCCCN(C)C*]

In some embodiments, L is

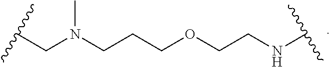 [SMILES: *CN(C)CCCOCCN*]

In some embodiments, L is

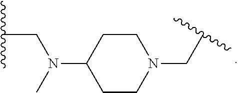 [SMILES: *CN1CCC(N(C)C*)CC1]

In some embodiments, L is

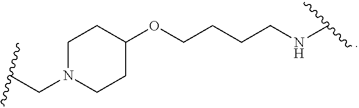 [SMILES: *CN1CCC(OCCCCN*)CC1]

In some embodiments, L is

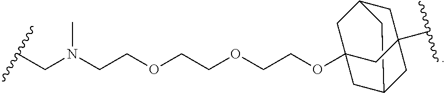 [SMILES: *CN(C)CCOCCOCCOC12CC3CC(CC(*)(C3)C1)C2]

In some embodiments, L is

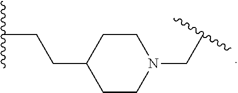 [SMILES: *CCC1CCN(C*)CC1]

In some embodiments, L is

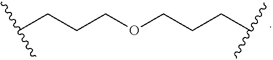 [SMILES: *CCCOCCC*]

In some embodiments, L is

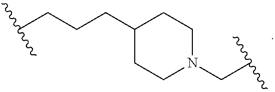 [SMILES: *CCCC1CCN(C*)CC1]

In some embodiments, L is

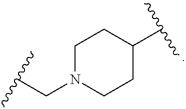 [SMILES: *CN1CCC(*)CC1]

In some embodiments, L is

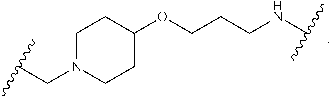 [SMILES: *CN1CCC(OCCCN*)CC1]

In some embodiments, L is

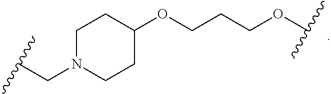 [SMILES: *CN1CCC(OCCCO*)CC1]

In some embodiments, L is

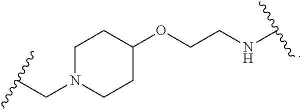 [SMILES: *CN1CCC(OCCN*)CC1]

In some embodiments, L is

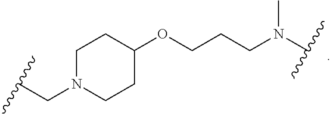 [SMILES: *CN1CCC(OCCCN(*)C)CC1]

In some embodiments, L is

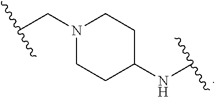 [SMILES: *CN1CCC(N*)CC1]

In some embodiments, L is

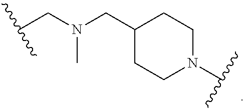 [SMILES: *CN(C)CC1CCN(*)CC1]

In some embodiments, L is

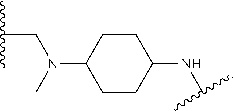 [SMILES: *CN(C)C1CCC(N*)CC1]

In some embodiments, L is

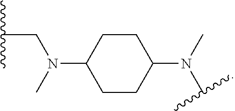 [SMILES: *CN(C)C1CCC(N(*)C)CC1]

In some embodiments, L is

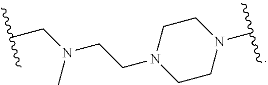 [SMILES: *CN(C)CCN1CCN(*)CC1]

In some embodiments, L is

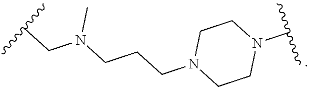 [SMILES: *CN(C)CCCN1CCN(*)CC1]

In some embodiments, L is

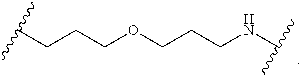 [SMILES: *CCCOCCCN*]

In some embodiments, L is

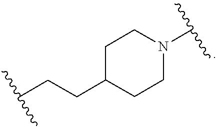 [SMILES: *CCC1CCN(*)CC1]

In some embodiments L is

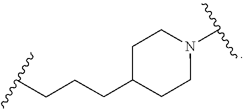 [SMILES: *CCCC1CCN(*)CC1]

In some embodiments, L is

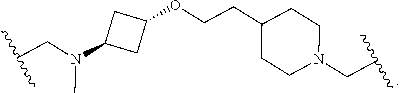 [SMILES: *CN1CCC(CCO[C@H]2C[C@H](N(C)C*)C2)CC1]

In some embodiments, L is

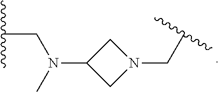 [SMILES: *CN1CC(N(C)C*)C1]

In some embodiments, L is

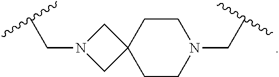 [SMILES: *CN1CCC2(CC1)CN(C*)C2]

In some embodiments, L is

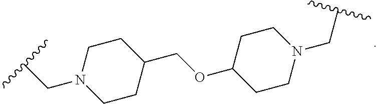 [SMILES: *CN1CCC(COC2CCN(C*)CC2)CC1]

In some embodiments, L is

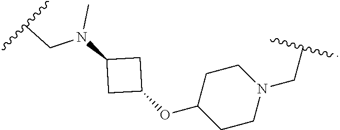 [SMILES: *CN1CCC(O[C@H]2C[C@H](N(C)C*)C2)CC1]

In some embodiments, L is

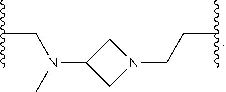 [SMILES: *CCN1CC(N(C)C*)C1]

In some embodiments, L is

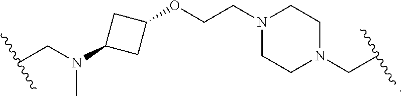 [SMILES: *CN1CCN(CCO[C@H]2C[C@H](N(C)C*)C2)CC1]

In some embodiments, L is

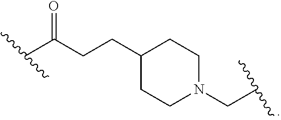 [SMILES: *CN1CCC(CCC(*)=O)CC1]

In some embodiments, L is

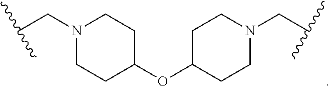 [SMILES: *CN1CCC(OC2CCN(C*)CC2)CC1]

In some embodiments, L is

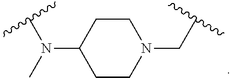 [SMILES: *CN1CCC(N(*)C)CC1]

In some embodiments, L is

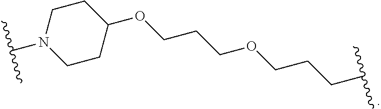 [SMILES: *CCCOCCCOC1CCN(*)CC1]

In some embodiments, L is

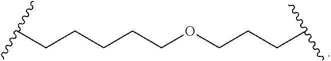 [SMILES: *CCCCCOCCC*]

In some embodiments, L is

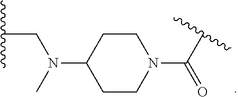 [SMILES: *CN(C)C1CCN(C(*)=O)CC1]

In some embodiments, L is

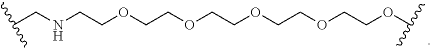 [SMILES: *CNCCOCCOCCOCCOCCO*]

In some embodiments, L is

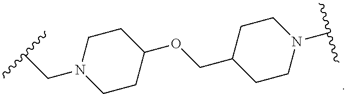 [SMILES: *CN1CCC(OCC2CCN(*)CC2)CC1]

In some embodiments, L is

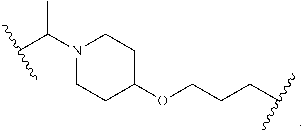 [SMILES: *CCCOC1CCN(C(*)C)CC1]

In some embodiments, L is

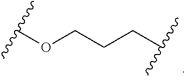 [SMILES: *CCCO*]

some embodiments, L is

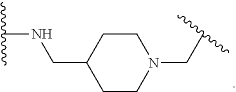 [SMILES: *CN1CCC(CN*)CC1]

In some embodiments, L is

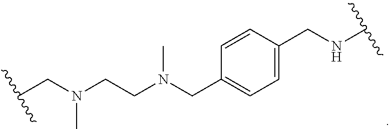 [SMILES: *CN(C)CCN(C)Cc1ccc(CN*)cc1]

In some embodiments, L is

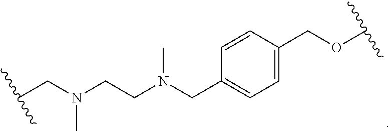 [SMILES: *CN(C)CCN(C)Cc1ccc(CO*)cc1]

In some embodiments, L is

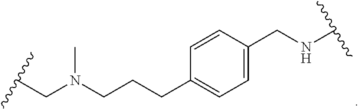 [SMILES: *CN(C)CCCc1ccc(CN*)cc1]

In some embodiments, L is

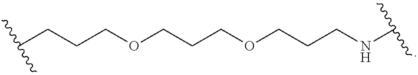 [SMILES: *CCCOCCCOCCCN*]

In some embodiments, L is

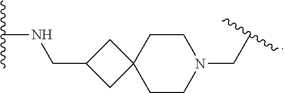 [SMILES: *CN1CCC2(CC1)CC(CN*)C2]

In some embodiments, L is

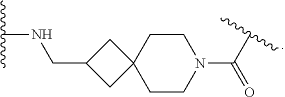 [SMILES: *NCC1CC2(CCN(C(*)=O)CC2)C1]

In some embodiments, L is

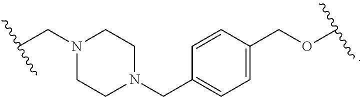 [SMILES: *CN1CCN(Cc2ccc(CO*)cc2)CC1]

In some embodiments, L is

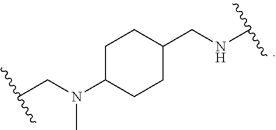 [SMILES: *CN(C)C1CCC(CN*)CC1]

In some embodiments, L is

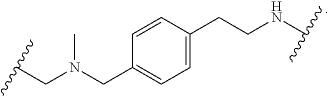 [SMILES: *CN(C)Cc1ccc(CCN*)cc1]

In some embodiments, L is

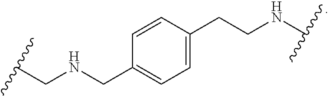 [SMILES: *CNCc1ccc(CCN*)cc1]

In some embodiments, L is

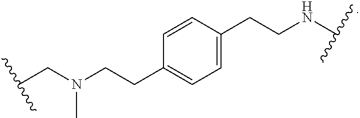 [SMILES: *CN(C)CCc1ccc(CCN*)cc1]

In some embodiments, L is

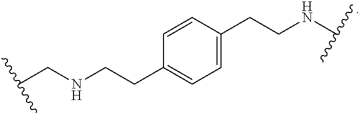 [SMILES: *CNCCc1ccc(CCN*)cc1]

In some embodiments, L is

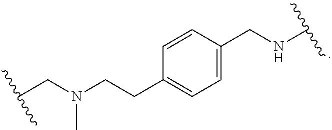 [SMILES: *CN(C)CCc1ccc(CN*)cc1]

In some embodiments, L is

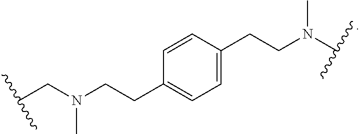 [SMILES: *CN(C)CCc1ccc(CCN(*)C)cc1]

In some embodiments, L is

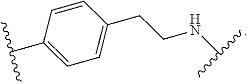 [SMILES: *NCCc1ccc(*)cc1]

In some embodiments, L is

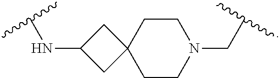 [SMILES: *CN1CCC2(CC1)CC(N*)C2]

In some embodiments, L is

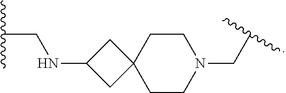 [SMILES: *CNC1CC2(CCN(C*)CC2)C1]

In some embodiments, L is

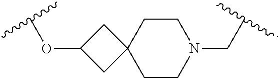 [SMILES: *CN1CCC2(CC1)CC(O*)C2]

In some embodiments, L is

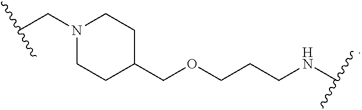 [SMILES: *CN1CCC(COCCCN*)CC1]

In some embodiments, L is

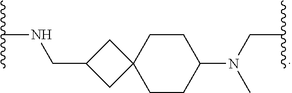 [SMILES: *CN(C)C1CCC2(CC1)CC(CN*)C2]

In some embodiments, L is

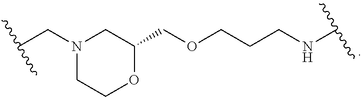 [SMILES: *CN1CCO[C@@H](COCCCN*)C1]

In some embodiments L is

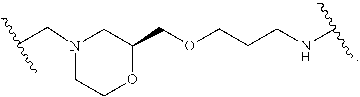 [SMILES: *CN1CCO[C@H](COCCCN*)C1]

In some embodiments L is

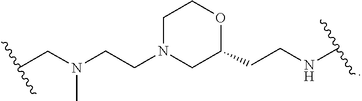 [SMILES: *CN(C)CCN1CCO[C@H](CCN*)C1]

In some embodiments, L is

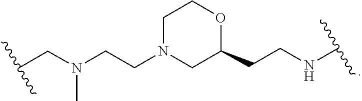 [SMILES: *CN(C)CCN1CCO[C@@H](CCN*)C1]

In some embodiments, L is

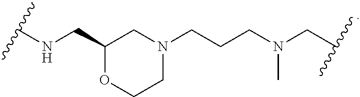 [SMILES: *CN(C)CCCN1CCO[C@@H](CN*)C1]

In some embodiments, L is

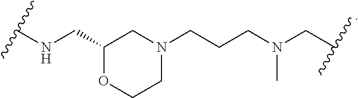 [SMILES: *CN(C)CCCN1CCO[C@H](CN*)C1]

In some embodiments, L is

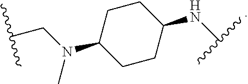 [SMILES: *CN(C)[C@H]1CC[C@@H](N*)CC1]

In some embodiments, L is

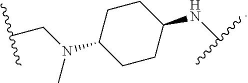 [SMILES: *CN(C)[C@H]1CC[C@H](N*)CC1]

In some embodiments, L is

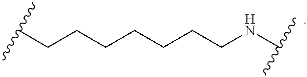 [SMILES: *CCCCCCCN*]

In some embodiments, L is

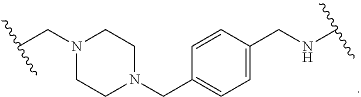 [SMILES: *CN1CCN(Cc2ccc(CN*)cc2)CC1]

In some embodiments, L is

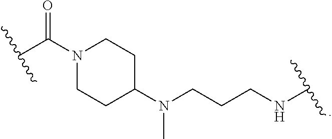 [SMILES: *NCCCN(C)C1CCN(C(*)=O)CC1]

In some embodiments, L is

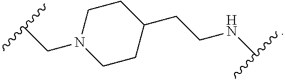 [SMILES: *CN1CCC(CCN*)CC1]

In some embodiments, L is

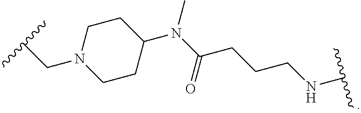 [SMILES: *CN1CCC(N(C)C(=O)CCCN*)CC1]

In some embodiments, L is

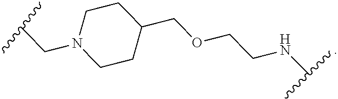 [SMILES: *CN1CCC(COCCN*)CC1]

In some embodiments, L is

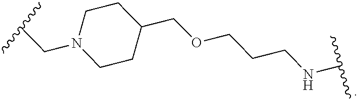 [SMILES: *CN1CCC(COCCCN*)CC1]

In some embodiments, L is

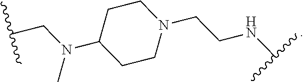 [SMILES: *CN(C)C1CCN(CCN*)CC1]

In some embodiments, L is

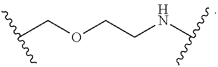 [SMILES: *COCCN*]

In some embodiments, L is

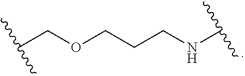 [SMILES: *COCCCN*]

In some embodiments, L is

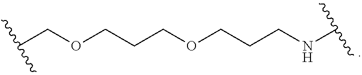 [SMILES: *COCCCOCCCN*]

In some embodiments, L is

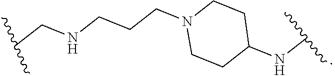 [SMILES: *CNCCCN1CCC(N*)CC1]

In some embodiments, L is

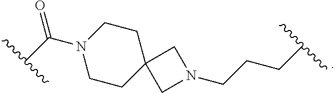 [SMILES: *CCCN1CC2(CCN(C(*)=O)CC2)C1]

In some embodiments, L is

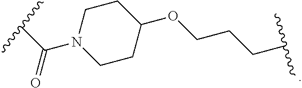 [SMILES: *CCCOC1CCN(C(*)=O)CC1]

In some embodiments, L is

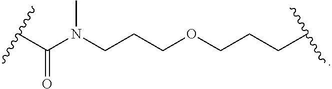 [SMILES: *CCCOCCCN(C)C(*)=O]

In some embodiments, L is

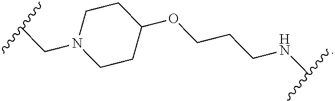 [SMILES: *CN1CCC(OCCCN*)CC1]

In some embodiments, L is

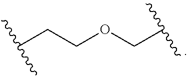 [SMILES: *CCOC*]

In some embodiments, L is

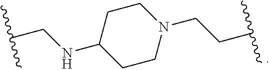 [SMILES: *CCN1CCC(NC*)CC1]

In some embodiments, L is

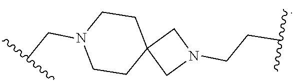 [SMILES: *CCN1CC2(CCN(C*)CC2)C1]

In some embodiments, L is

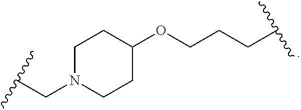 [SMILES: *CCCOC1CCN(C*)CC1]

In some embodiments, L is

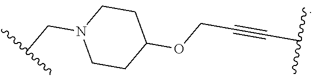 [SMILES: *C#CCOC1CCN(C*)CC1]

In some embodiments, L is

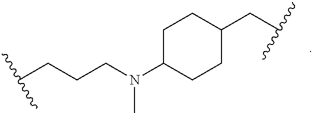 [SMILES: *CCCN(C)C1CCC(C*)CC1]

In some embodiments, L is

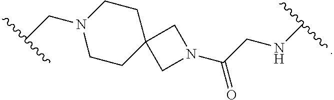 [SMILES: *CN1CCC2(CC1)CN(C(=O)CN*)C2]

In some embodiments, L is

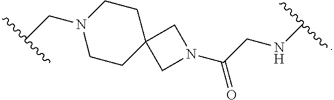 [SMILES: *CN1CCC2(CC1)CN(C(=O)CN*)C2]

In some embodiments, L is

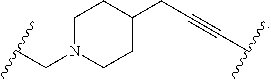 [SMILES: *C#CCC1CCN(C*)CC1]

In some embodiments, L is

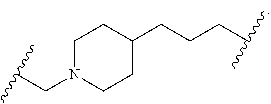 [SMILES: *CCCC1CCN(C*)CC1]

In some embodiments, L is

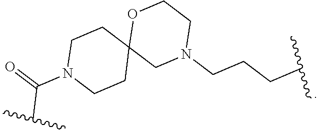 [SMILES: *CCCN1CCOC2(CCN(C(*)=O)CC2)C1]

In some embodiments, L is

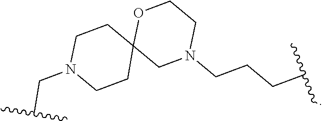 [SMILES: *CCCN1CCOC2(CCN(C*)CC2)C1]

In some embodiments, L is

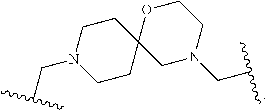 [SMILES: *CN1CCC2(CC1)CN(C*)CCO2]

In some embodiments, L is

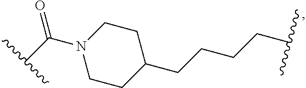 [SMILES: *CCCCC1CCN(C(*)=O)CC1]

In some embodiments, L is

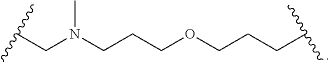 [SMILES: *CCCOCCCN(C)C*]

In some embodiments, L is

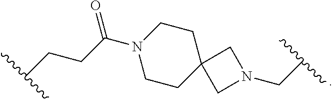 [SMILES: *CCC(=O)N1CCC2(CC1)CN(C*)C2]

In some embodiments, L is

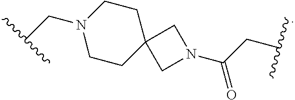 [SMILES: *CC(=O)N1CC2(CCN(C*)CC2)C1]

In some embodiments, L is

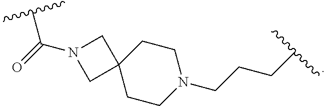 [SMILES: *CCCN1CCC2(CC1)CN(C(*)=O)C2]

In some embodiments, L is

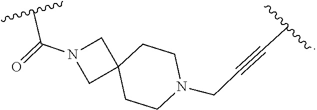 [SMILES: *C#CCN1CCC2(CC1)CN(C(*)=O)C2]

In some embodiments, L is

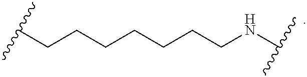 [SMILES: *CCCCCCCN*]

In some embodiments, L is

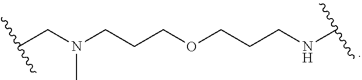 [SMILES: *CN(C)CCCOCCCN*]

In some embodiments, L is

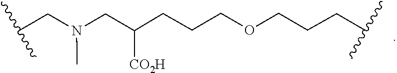 [SMILES: *CCCOCCCC(CN(C)C*)C(=O)O]

In some embodiments, L is

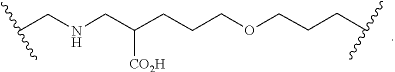 [SMILES: *CCCOCCCC(CNC*)C(=O)O]

In some embodiments, L is

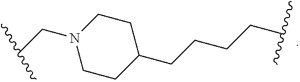 [SMILES: *CCCCC1CCN(C*)CC1]

In some embodiments, L is

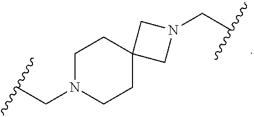 [SMILES: *CN1CCC2(CC1)CN(C*)C2]

In some embodiments, L is

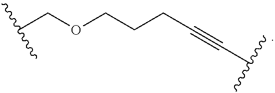 [SMILES: *C#CCCCOC*]

In some embodiments, L is

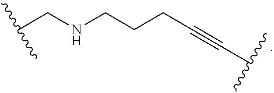 [SMILES: *C#CCCCNC*]

In some embodiments, L is

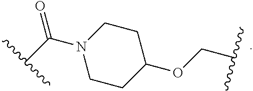 [SMILES: *COC1CCN(C(*)=O)CC1]

In some embodiments, L is

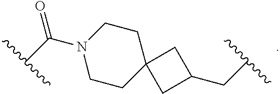 [SMILES: *CC1CC2(CCN(C(*)=O)CC2)C1]

In some embodiments, L is

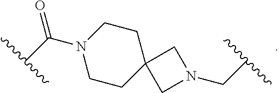 [SMILES: *CN1CC2(CCN(C(*)=O)CC2)C1]

In some embodiments, L is

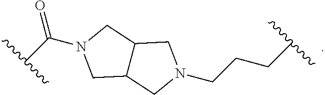 [SMILES: *CCCN1CC2CN(C(*)=O)CC2C1]

In some embodiments, L is

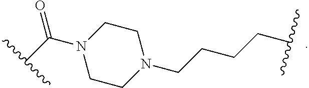 [SMILES: *CCCCN1CCN(C(*)=O)CC1]

In some embodiments, L is

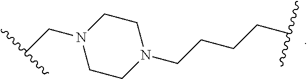 [SMILES: *CCCCN1CCN(C*)CC1]

In some embodiments, L is

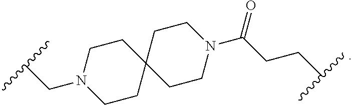 [SMILES: *CCC(=O)N1CCC2(CCN(C*)CC2)CC1]

In some embodiments, L is

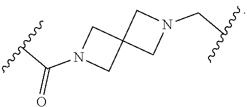 [SMILES: *CN1CC2(C1)CN(C(*)=O)C2]

In some embodiments, L is

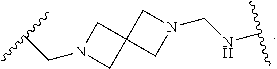 [SMILES: *CN1CC2(C1)CN(CN*)C2]

In some embodiments, L is

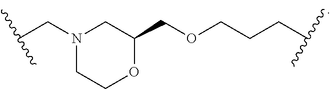 [SMILES: *CCCOC[C@@H]1CN(C*)CCO1]

In some embodiments, L is

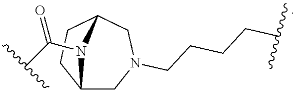 [SMILES: *CCCCN1C[C@H]2CC[C@@H](C1)N2C(*)=O]

In some embodiments, L is

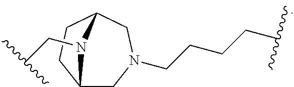 [SMILES: *CCCCN1C[C@H]2CC[C@@H](C1)N2C*]

In some embodiments, L is

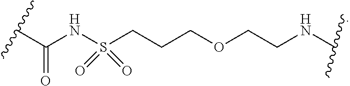 [SMILES: *NCCOCCCS(=O)(=O)NC(*)=O]

In some embodiments, L is

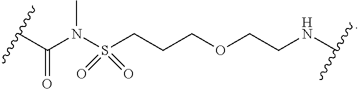 [SMILES: *NCCOCCCS(=O)(=O)N(C)C(*)=O]

In some embodiments, L is

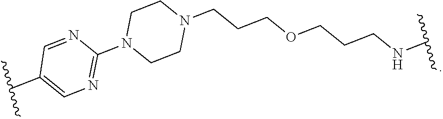 [SMILES: *NCCCOCCCN1CCN(c2ncc(*)cn2)CC1]

In some embodiments, L is

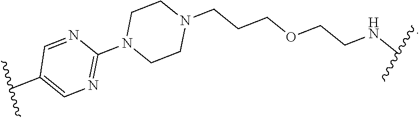 [SMILES: *NCCOCCCN1CCN(c2ncc(*)cn2)CC1]

In some embodiments, L is

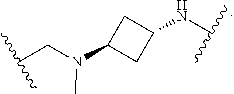 [SMILES: *CN(C)[C@H]1C[C@H](N*)C1]

In some embodiments, L is

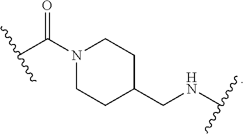 [SMILES: *NCC1CCN(C(*)=O)CC1]

In some embodiments, L is

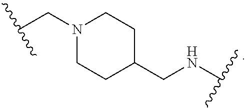 [SMILES: *CN1CCC(CN*)CC1]

In some embodiments, L is

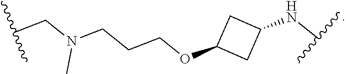 [SMILES: *CN(C)CCCO[C@H]1C[C@H](N*)C1]

In some embodiments, L is

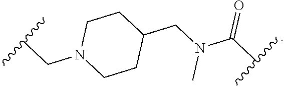 [SMILES: *CN1CCC(CN(C)C(*)=O)CC1]

In some embodiments, L is

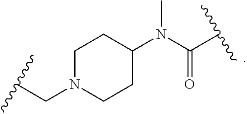 [SMILES: *CN1CCC(N(C)C(*)=O)CC1]

In some embodiments, L is

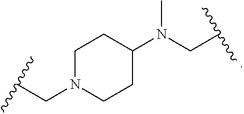 [SMILES: *CN1CCC(N(C)C*)CC1]

In some embodiments, L is

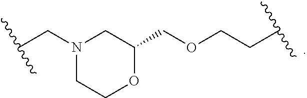 [SMILES: *CCOC[C@H]1CN(C*)CCO1]

In some embodiments, L is

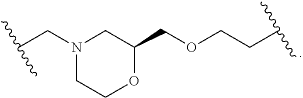 [SMILES: *CCOC[C@@H]1CN(C*)CCO1]

In some embodiments, L is

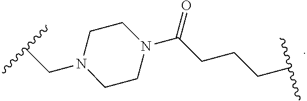 [SMILES: *CCCC(=O)N1CCN(C*)CC1]

In some embodiments, L is

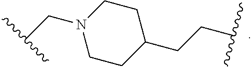 [SMILES: *CCC1CCN(C*)CC1]

In some embodiments, L is

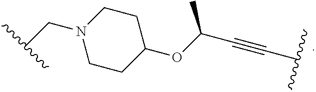 [SMILES: *C#C[C@H](C)OC1CCN(C*)CC1]

In some embodiments, L is

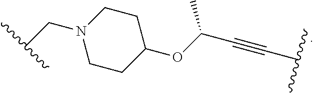 [SMILES: *C#C[C@@H](C)OC1CCN(C*)CC1]

In some embodiments, L is

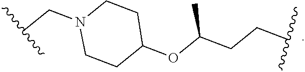 [SMILES: *CC[C@H](C)OC1CCN(C*)CC1]

In some embodiments, L is

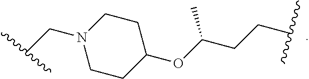 [SMILES: *CC[C@@H](C)OC1CCN(C*)CC1 ; "."]

In some embodiments, L is

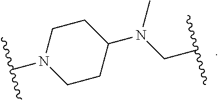 [SMILES: *CN(C)C1CCN(*)CC1]

In some embodiments, L is

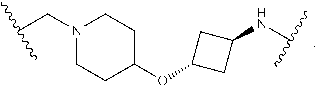 [SMILES: *CN1CCC(O[C@H]2C[C@H](N*)C2)CC1]

In some embodiments, L is

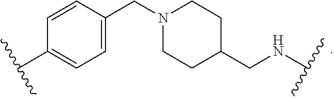 [SMILES: *NCC1CCN(Cc2ccc(*)cc2)CC1]

In some embodiments, L is

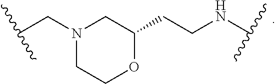 [SMILES: *CN1CCO[C@@H](CCN*)C1]

In some embodiments, L is

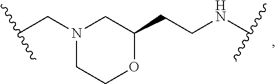 [SMILES: *CN1CCO[C@H](CCN*)C1]

. In some embodiments, L is

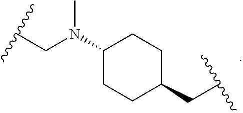 [SMILES: *CN(C)[C@H]1CC[C@H](C*)CC1]

In some embodiments, L is

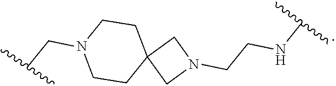 [SMILES: *CN1CCC2(CC1)CN(CCN*)C2]

In some embodiments, L is

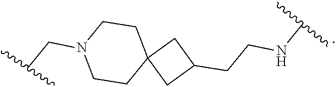 [SMILES: *CN1CCC2(CC1)CC(CCN*)C2]

In some embodiments, L is

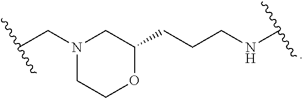 [SMILES: *CN1CCO[C@@H](CCCN*)C1]

In some embodiments, L is

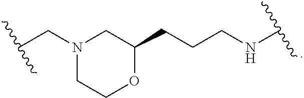 [SMILES: *CN1CCO[C@H](CCCN*)C1]

In some embodiments, L is

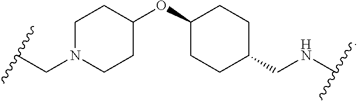 [SMILES: *CN1CCC(O[C@H]2CC[C@H](CN*)CC2)CC1]

In some embodiments, L is

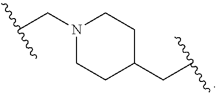 [SMILES: *CC1CCN(C*)CC1]

In some embodiments, L is

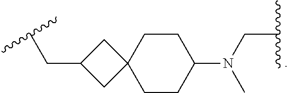 [SMILES: *CC1CC2(CCC(N(C)C*)CC2)C1]

In some embodiments, L is

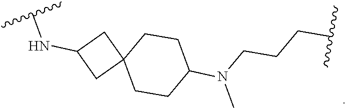 [SMILES: *CCCN(C)C1CCC2(CC1)CC(N*)C2]

In some embodiments, L is

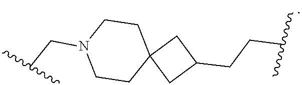 [SMILES: *CCC1CC2(CCN(C*)CC2)C1]

In some embodiments, L is

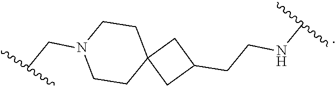 [SMILES: *CN1CCC2(CC1)CC(CCN*)C2]

In some embodiments, L is

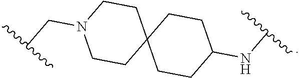 [SMILES: *CN1CCC2(CCC(N*)CC2)CC1]

In some embodiments, L is

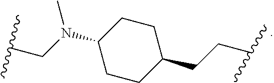 [SMILES: *CC[C@H]1CC[C@H](N(C)C*)CC1]

In some embodiments, L is

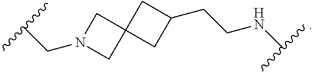 [SMILES: *CN1CC2(CC(CCN*)C2)C1]

In some embodiments, L is

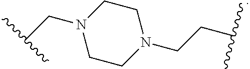 [SMILES: *CCN1CCN(C*)CC1 ; "."]

In some embodiments, L is

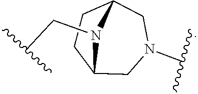 [SMILES: *CN1[C@@H]2CC[C@H]1CN(*)C2]

In some embodiments, L is

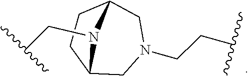 [SMILES: *CCN1C[C@H]2CC[C@@H](C1)N2C*]

In some embodiments, L is

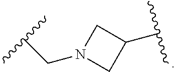 [SMILES: *CN1CC(*)C1]

In some embodiments, L is

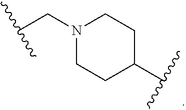 [SMILES: *CN1CCC(*)CC1]

In some embodiments, L is

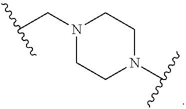 [SMILES: *CN1CCN(*)CC1]

In some embodiments, L is

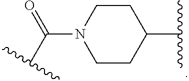 [SMILES: *C(=O)N1CCC(*)CC1]

In some embodiments, L is

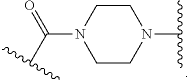 [SMILES: *C(=O)N1CCN(*)CC1]

some embodiments, L is

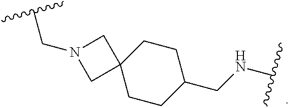 [SMILES: *CN1CC2(CCC(CN*)CC2)C1]

In some embodiments, L is

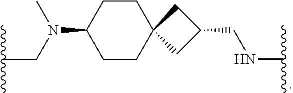

In some embodiments, L is

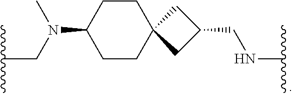

In some embodiments, L is

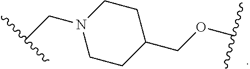 [SMILES: *CN1CCC(CO*)CC1]

In some embodiments, L is

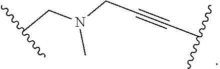 [SMILES: *C#CCN(C)C*]

In some embodiments, L is

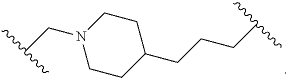 [SMILES: *CCCC1CCN(C*)CC1]

In some embodiments, L is

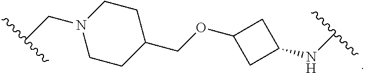

In some embodiments, L is

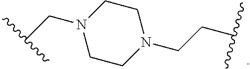 [SMILES: *CCN1CCN(C*)CC1]

In some embodiments, L is

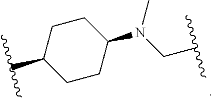 [SMILES: *CN(C)[C@H]1CC[C@@H](*)CC1]

In some embodiments, L is

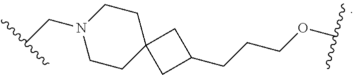 [SMILES: *CN1CCC2(CC1)CC(CCCO*)C2]

In some embodiments, L is

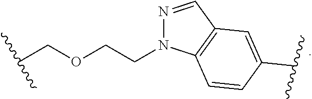 [SMILES: *COCCn1ncc2cc(*)ccc21]

In some embodiments, L is

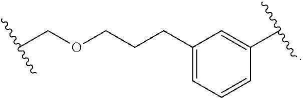 [SMILES: *COCCCc1cccc(*)c1]

In some embodiments, L is

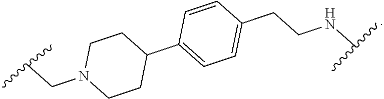 [SMILES: *CN1CCC(c2ccc(CCN*)cc2)CC1]

In some embodiments, L is

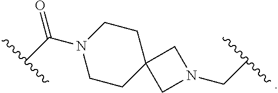 [SMILES: *CN1CC2(CCN(C(*)=O)CC2)C1]

In some embodiments, L is

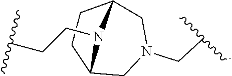 [SMILES: *CCN1[C@@H]2CC[C@H]1CN(C*)C2]

In some embodiments, L is

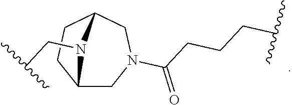 [SMILES: *CCCC(=O)N1C[C@H]2CC[C@@H](C1)N2C*]

In some embodiments, L is

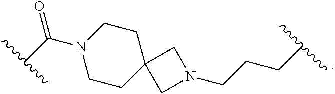 [SMILES: *CCCN1CC2(CCN(C(*)=O)CC2)C1]

In some embodiments, L is

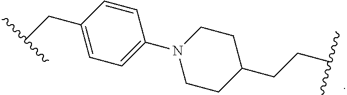 [SMILES: *CCC1CCN(c2ccc(C*)cc2)CC1]

In some embodiments, L is

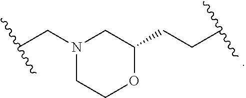 [SMILES: *CC[C@H]1CN(C*)CCO1]

In some embodiments, L is

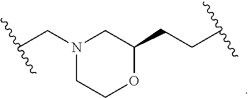 [SMILES: *CC[C@@H]1CN(C*)CCO1]

In some embodiments, L is

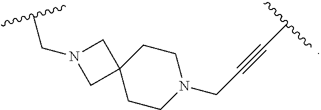 [SMILES: *C#CCN1CCC2(CC1)CN(C*)C2]

In some embodiments, L is

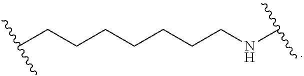 [SMILES: *CCCCCCCN*]

In some embodiments, L is

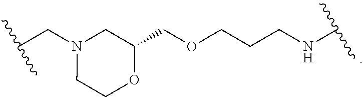 [SMILES: *CN1CCO[C@@H](COCCCN*)C1]

In some embodiments, L is

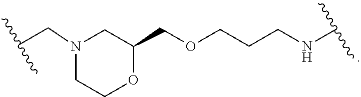 [SMILES: *CN1CCO[C@H](COCCCN*)C1]

In some embodiments, L is

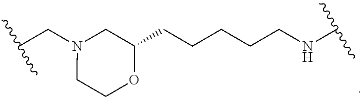 [SMILES: *CN1CCO[C@@H](CCCCCN*)C1]

In some embodiments, L is

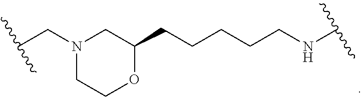 [SMILES: *CN1CCO[C@H](CCCCCN*)C1]

In some embodiments, L is

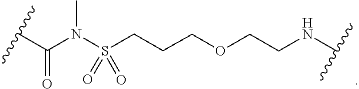 [SMILES: *NCCOCCCS(=O)(=O)N(C)C(*)=O]

In some embodiments, L is

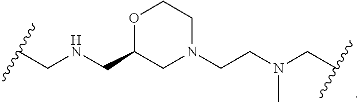 [SMILES: *CNC[C@@H]1CN(CCN(C)C*)CCO1]

In some embodiments, L is

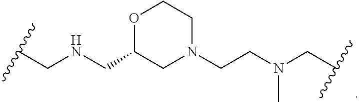 [SMILES: *CNC[C@H]1CN(CCN(C)C*)CCO1]

In some embodiments L is

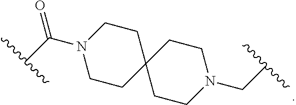 [SMILES: *CN1CCC2(CC1)CCN(C(*)=O)CC2]

In some embodiments, L is

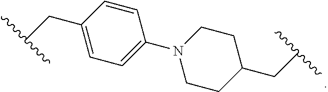 [SMILES: *Cc1ccc(N2CCC(C*)CC2)cc1]

In some embodiments, L is

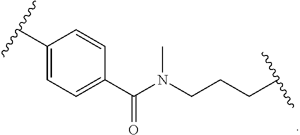 [SMILES: *CCCN(C)C(=O)c1ccc(*)cc1]

In some embodiments, L is

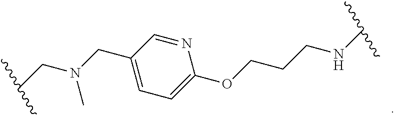 [SMILES: *CN(C)Cc1ccc(OCCCN*)nc1]

In some embodiments, L is

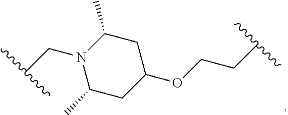 [SMILES: *CCOC1C[C@@H](C)N(C*)[C@@H](C)C1]

In some embodiments L is

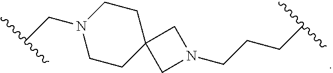 [SMILES: *CCCN1CC2(CCN(C*)CC2)C1]

In some embodiments, L is

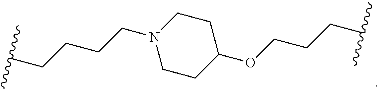 [SMILES: *CCCCN1CCC(OCCC*)CC1]

In some embodiments, L is

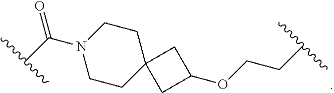 [SMILES: *CCOC1CC2(CCN(C(*)=O)CC2)C1]

In some embodiments, L is

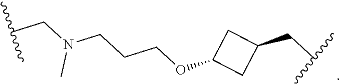 [SMILES: *CN(C)CCCO[C@H]1C[C@H](C*)C1]

In some embodiments, L is

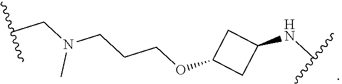 [SMILES: *CN(C)CCCO[C@H]1C[C@H](N*)C1]

In some embodiments, L is

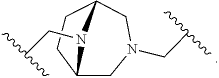 [SMILES: *CN1C[C@H]2CC[C@@H](C1)N2C*]

In some embodiments, L is

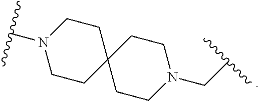 [SMILES: *CN1CCC2(CCN(*)CC2)CC1]

In some embodiments, L is

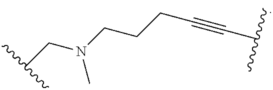 [SMILES: *C#CCCCN(C)C*]

In some embodiments, L is

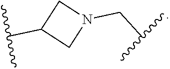 [SMILES: *CN1CC(*)C1]

In some embodiments, L is

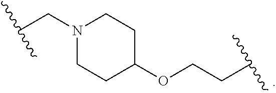 [SMILES: *CCOC1CCN(C*)CC1]

In some embodiments, L is

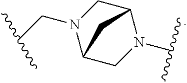 [SMILES: *CN1C[C@H]2C[C@@H]1CN2*]

In some embodiments, L is

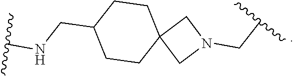 [SMILES: *CN1CC2(CCC(CN*)CC2)C1]

In some embodiments, L is

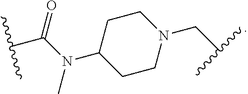 [SMILES: *CN1CCC(N(C)C(*)=O)CC1]

In some embodiments, L is

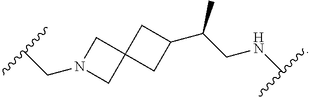 [SMILES: *CN1CC2(CC([C@@H](C)CN*)C2)C1]

In some embodiments, L is

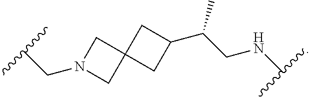 [SMILES: *CN1CC2(CC([C@H](C)CN*)C2)C1]

In some embodiments, L is

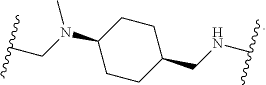 [SMILES: *CN(C)[C@H]1CC[C@@H](CN*)CC1]

In some embodiments, L is

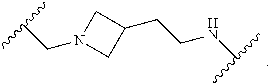 [SMILES: *CN1CC(CCN*)C1]

N In some embodiments, L is

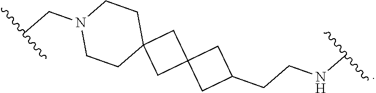 [SMILES: *CN1CCC2(CC1)CC1(CC(CCN*)C1)C2]

In some embodiments, L is

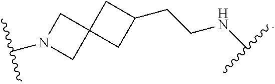 [SMILES: *NCCC1CC2(C1)CN(*)C2]

In some embodiments, L is selected from the embodiments of “L” described in WO 2021/127190; WO 2021/011634; WO 2021/178920; WO 2021/127443; and WO 2018/071606; the contents of each of which are hereby incorporated by reference. In some embodiments, L is selected from the embodiments of “linker” described in WO2021/083949; the content of which is hereby incorporated by reference.

In some embodiments, L is selected from the groups depicted in the compounds in Table 1, below.

When L is described without specifying the point of attachment to PIK or BM, PIK and BM can be connected at either point of attachment of L. For example, when L is

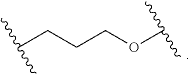 [SMILES: *CCCO*]

the resulting compounds of formula I are represented by the following formulae

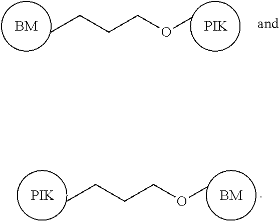

### Binding Moiety (BM)

As defined generally above and described herein, BM is a binding motif LBM, PIK2, or T. In some embodiments, BM is LBM or PIK2. In some embodiments, BM is LBM. In some embodiments, BM is PIK2. In some embodiments, BM is T. In some embodiments, BM is selected from the groups depicted in the compounds in Table 1, below.

As defined generally above and described herein, LBM is an E3 ubiquitin ligase binding moiety. In some embodiments, LBM is an E3 ligase ligand well known to one of ordinary skill in the art including those described in M. Toure, C. M. Crews, Angew. Chem. Int. Ed. 2016, 55, 1966; T. Uehara et al. Nature Chemical Biology 2017, 13, 675; WO 2017/176708; US 2017/0281784; WO 2017/161119; WO 2017/176957; WO 2017/176958; WO 2015/160845; US 2015/0291562; WO 2016/197032; WO 2016/105518; US 2018/0009779; WO 2017/007612; WO 2018/0134684; WO 2013/106643; US 2014/0356322; WO 2002/020740; US 2002/0068063; WO 2012/078559; US 2014/0302523; WO 2012/003281; US 2013/0190340; US 2016/0022642; WO 2014/063061; US 2015/0274738; WO 2016/118666; US 2016/0214972; WO 2016/149668; US 2016/0272639; WO 2016/169989; US 2018/0118733; WO 2016/197114; US 2018/0147202; WO 2017/011371; US 2017/0008904; WO 2017/011590; US 2017/0037004; WO 2017/079267; US 2017/0121321; WO 2017/117473; WO 2017/117474; WO 2013/106646; WO 2014/108452; WO 2017/197036; US 2019/0076540; WO 2017/197046; US 2019/0076542; WO 2017/197051; US 2019/0076539; WO 2017/197055; US 2019/0076541; and WO 2017/197056; the entirety of each of which is herein incorporated by reference.

In some embodiments, LBM is a cereblon (CRBN) E3 ubiquitin ligase binding moiety. In some embodiments, LBM is an IMiD-based cereblon E3 ubiquitin ligase binding moiety.

In some embodiments, an IMiD-based cereblon E3 ligase binding moiety, including those disclosed and described herein, includes thalidomide, lenalidomide, pomalidomide, avadomide (CC-122), iberdomide (CC-220), CC-92480, CC-885, CC-9009, and analogs thereof, and those IMiD-based cereblon ligands described in WO 2002/059106, U.S. Pat. Nos. 7,629,360, 5,874,448, WO 2009/145899, WO 2009/042177, WO 1999/047512, WO 2008/039489, WO 2008/115516, WO 2009/139880, US 2011/0196150, WO 2008/027542, WO 1998/54170, WO 1999/46258, WO 2018/102725, and WO 2014/018866, the contents of each of which is herein incorporated by reference.

In some embodiments, LBM is

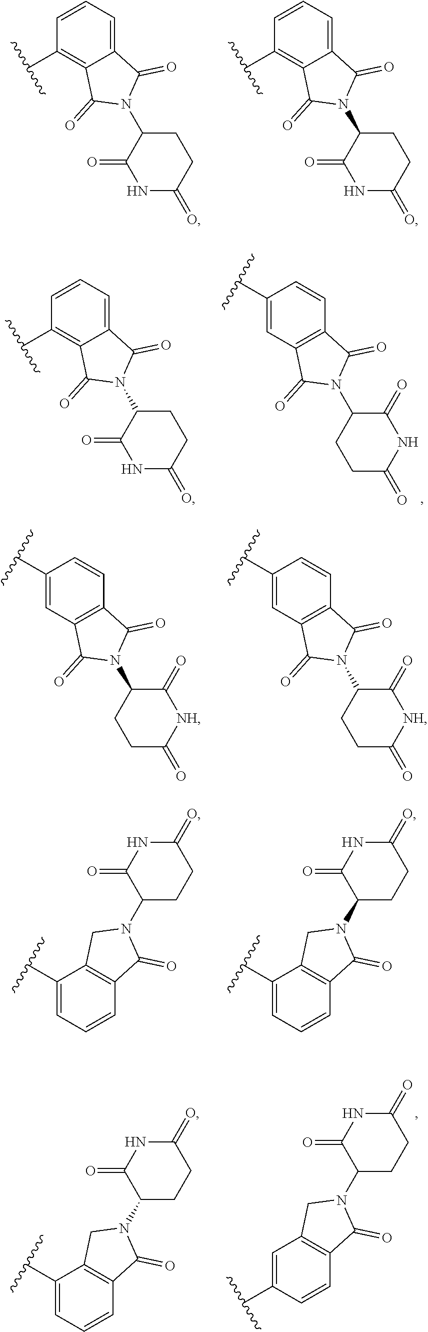

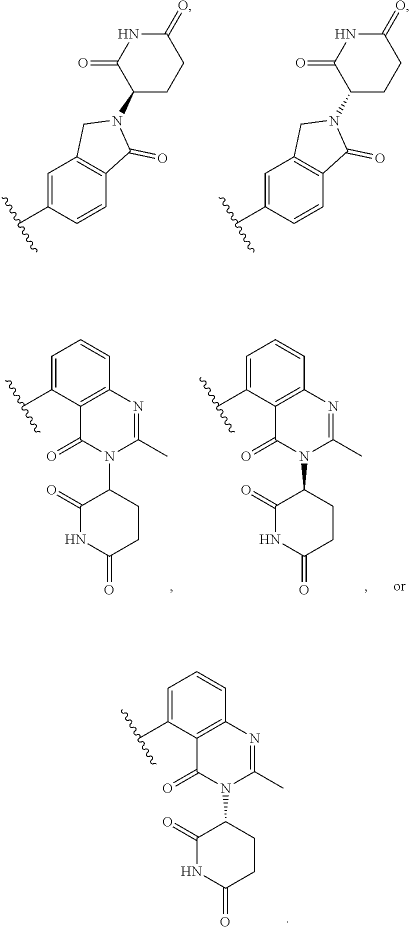 [SMILES: *c1ccc2c(c1)CN([C@@H]1CCC(=O)NC1=O)C2=O ; *c1ccc2c(c1)CN([C@H]1CCC(=O)NC1=O)C2=O ; *c1cccc2nc(C)n(C3CCC(=O)NC3=O)c(=O)c12 ; *c1cccc2nc(C)n([C@H]3CCC(=O)NC3=O)c(=O)c12 ; *c1cccc2nc(C)n([C@@H]3CCC(=O)NC3=O)c(=O)c12]

In some embodiments, LBM is

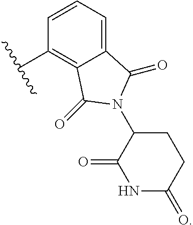 [SMILES: *c1cccc2c1C(=O)N(C1CCC(=O)NC1=O)C2=O]

In some embodiments, LBM is

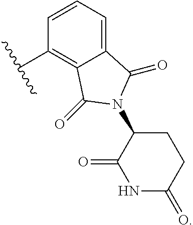 [SMILES: *c1cccc2c1C(=O)N([C@H]1CCC(=O)NC1=O)C2=O]

In some embodiments, LBM is

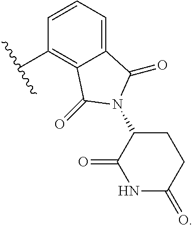

In some embodiments, LBM is

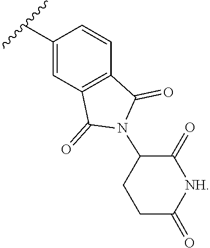 [SMILES: *c1ccc2c(c1)C(=O)N(C1CCC(=O)NC1=O)C2=O]

In some embodiments, LBM is

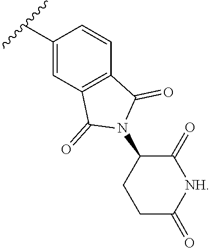 [SMILES: *c1ccc2c(c1)C(=O)N([C@@H]1CCC(=O)NC1=O)C2=O]

In some embodiments, LBM is

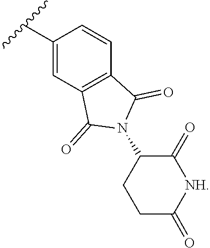

In some embodiments, LBM is

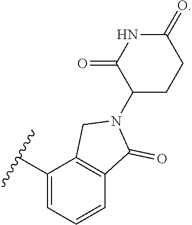 [SMILES: *c1cccc2c1CN(C1CCC(=O)NC1=O)C2=O]

In some embodiments, LBM is

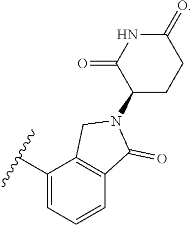 [SMILES: *c1cccc2c1CN([C@@H]1CCC(=O)NC1=O)C2=O]

In some embodiments, LBM is

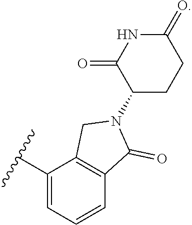 [SMILES: *c1cccc2c1CN([C@H]1CCC(=O)NC1=O)C2=O]

In some embodiments, LBM is

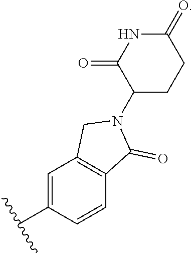 [SMILES: *c1ccc2c(c1)CN(C1CCC(=O)NC1=O)C2=O]

In some embodiments, LBM is

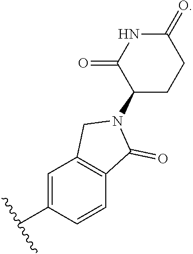 [SMILES: *c1ccc2c(c1)CN([C@@H]1CCC(=O)NC1=O)C2=O]

In some embodiments, LBM is

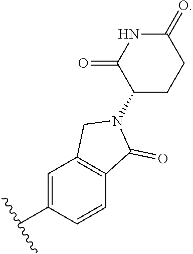 [SMILES: *c1ccc2c(c1)CN([C@H]1CCC(=O)NC1=O)C2=O]

In some embodiments, LBM is

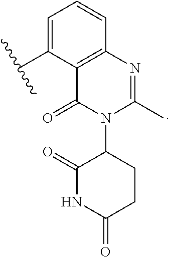 [SMILES: *c1cccc2nc(C)n(C3CCC(=O)NC3=O)c(=O)c12]

In some embodiments, LBM is

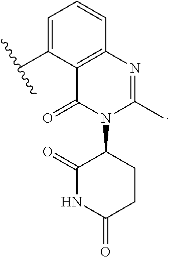 [SMILES: *c1cccc2nc(C)n([C@H]3CCC(=O)NC3=O)c(=O)c12]

In some embodiments, LBM is

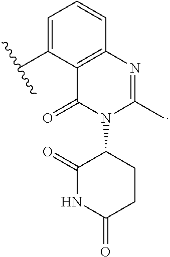 [SMILES: *c1cccc2nc(C)n([C@@H]3CCC(=O)NC3=O)c(=O)c12]

In some embodiments, LBM is a Von Hippel-Lindau (VHL) E3 ubiquitin ligase binding moiety. Von Hippel-Lindau (VHL) E3 ubiquitin ligase binding moieties include, for example, those described in US 2016/0045607, WO 2017/079267, WO 2018/102725, WO 2021/207172, the contents of each of which is herein incorporated by reference.

In some embodiments, LBM is

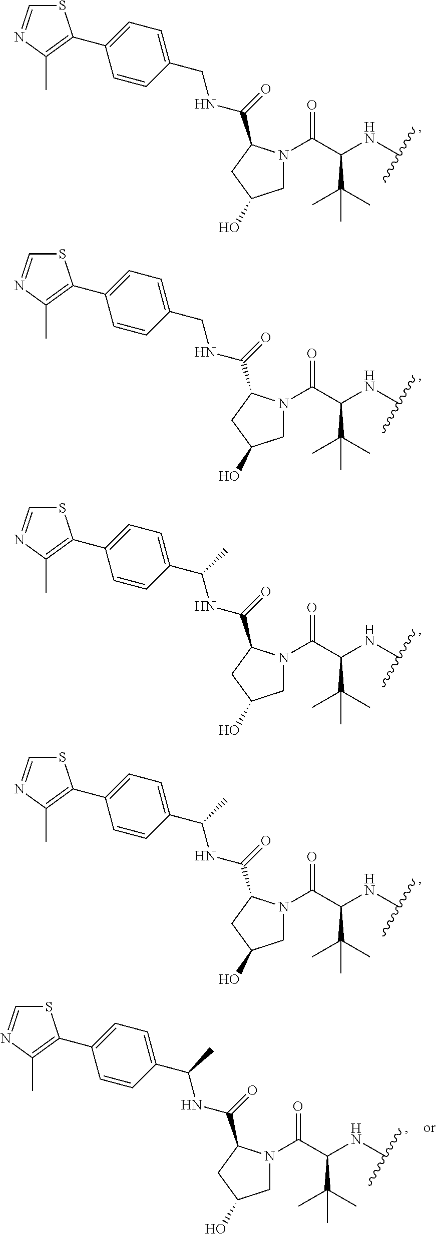 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@@H]1C(=O)NCc1ccc(-c2scnc2C)cc1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@@H](O)C[C@@H]1C(=O)N[C@@H](C)c1ccc(-c2scnc2C)cc1)C(C)(C)C ; *N[C@H](C(=O)N1C[C@H](O)C[C@H]1C(=O)N[C@H](C)c1ccc(-c2scnc2C)cc1)C(C)(C)C]

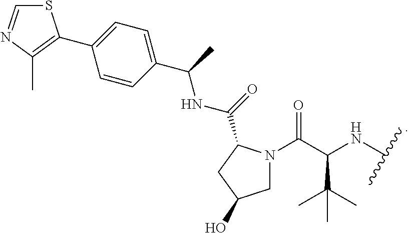 [SMILES: *N[C@H](C(=O)N1C[C@@H](O)C[C@@H]1C(=O)N[C@H](C)c1ccc(-c2scnc2C)cc1)C(C)(C)C]

In some embodiments, LBM is

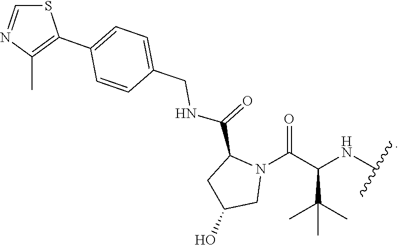 [SMILES: *N[C@H](C(=O)N1C[C@H](O)C[C@H]1C(=O)NCc1ccc(-c2scnc2C)cc1)C(C)(C)C]

In some embodiments, LBM is

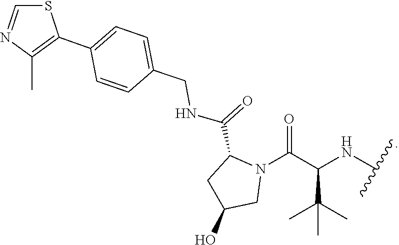 [SMILES: *N[C@H](C(=O)N1C[C@@H](O)C[C@@H]1C(=O)NCc1ccc(-c2scnc2C)cc1)C(C)(C)C]

In some embodiments, LBM is

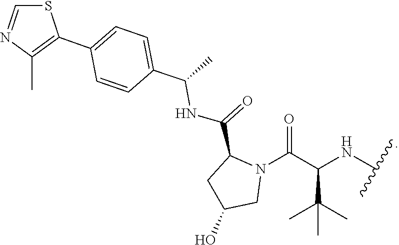 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

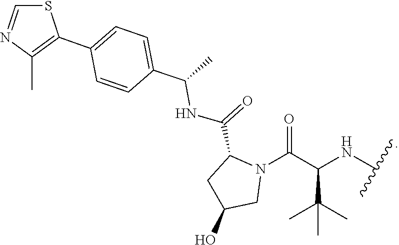 [SMILES: *N[C@H](C(=O)N1C[C@@H](O)C[C@@H]1C(=O)N[C@@H](C)c1ccc(-c2scnc2C)cc1)C(C)(C)C]

In some embodiments, LBM is

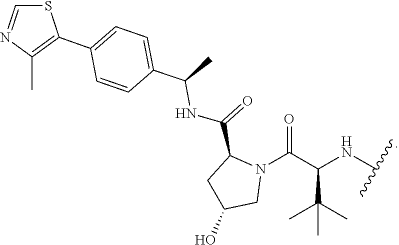 [SMILES: *N[C@H](C(=O)N1C[C@H](O)C[C@H]1C(=O)N[C@H](C)c1ccc(-c2scnc2C)cc1)C(C)(C)C]

In some embodiments, LBM is

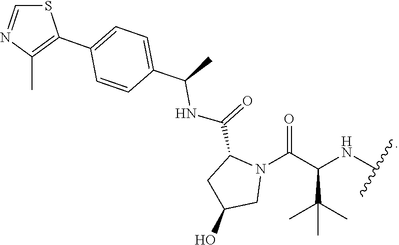 [SMILES: *N[C@H](C(=O)N1C[C@@H](O)C[C@@H]1C(=O)N[C@H](C)c1ccc(-c2scnc2C)cc1)C(C)(C)C]

In some embodiments, LBM is selected from the groups depicted in the compounds in Table 1, below.

As defined generally above, PIK2 is a second PI3K binding moiety capable of binding to PI3Kα. In some embodiments, PIK and PIK2 are different PI3K binding moieties capable of binding to PI3Kα. In some embodiments, PIK and PIK2 are the same PI3K binding moieties capable of binding to PI3Kα.

In some embodiments, PIK and PIK2 bind to the same location on PI3Kα. In some embodiments, PIK and PIK2 bind to the different locations on PI3Kα.

In some embodiments, PIK2 is as defined by PIK in embodiments and classes and subclasses herein. For example, in certain embodiments, PIK2 is a PI3K binding moiety of formula I-a0:

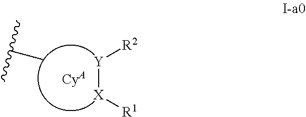

or a pharmaceutically acceptable salt thereof, wherein each of X, Y, Cy^{A}, R¹, and R² is as defined in embodiments and classes and subclasses herein. For example, in certain embodiments, PIK2 is a PI3K binding moiety of formula I-a0, wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA}; or
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloallylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, r¹, r², r³, r⁴, r⁵, and r⁶ is independently 0, 1, 2, 3, 4, or 5.

As another example, in certain embodiments, PIK2 is a PI3K binding moiety of formula I-a0, wherein R^{1A} is

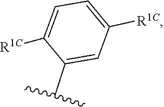

and R² is

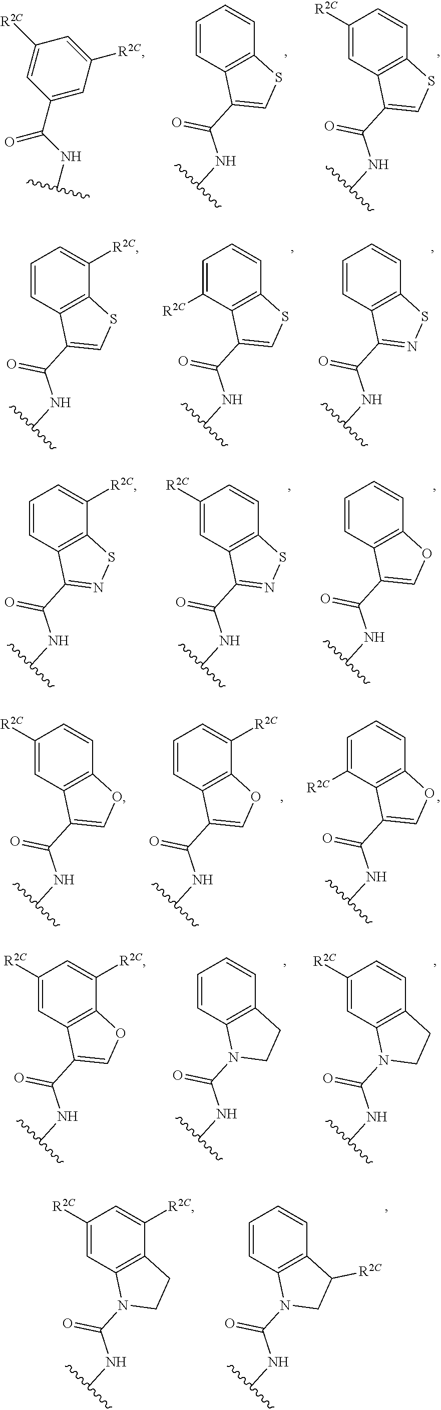

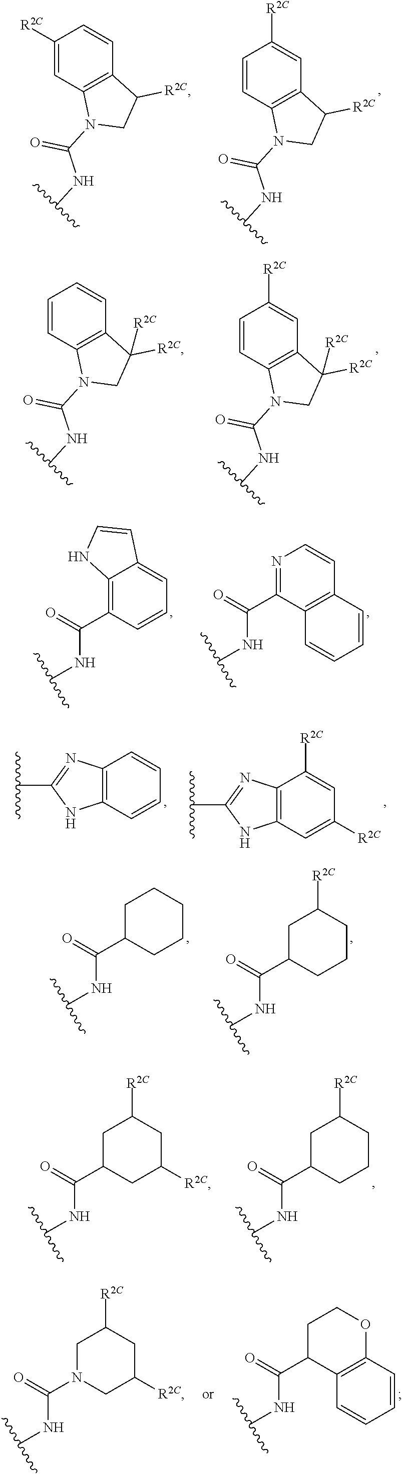

wherein each of R^{1C} and R^{2C} is as defined in embodiments and classes and subclasses herein.

In certain embodiments, PIK2 is a PI3K binding moiety of formula I-b0:

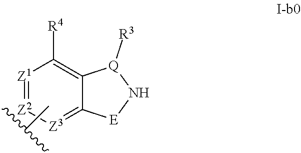

or a pharmaceutically acceptable salt thereof, wherein each of E, Q, R³, R⁴, Z¹, Z², and Z³ is as defined in embodiments and classes and subclasses herein.

In certain embodiments, PIK2 is a PI3K binding moiety of formula I-c0:

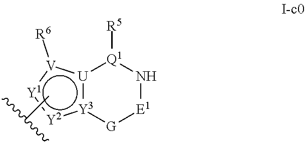

or a pharmaceutically acceptable salt thereof, wherein each of E¹, G, Q¹, R⁵, R⁶, U, V, Y¹, Y², and Y³ is as defined in embodiments and classes and subclasses herein.

In certain embodiments, PIK2 is a PI3K binding moiety of formula I-d0 or I-d00:

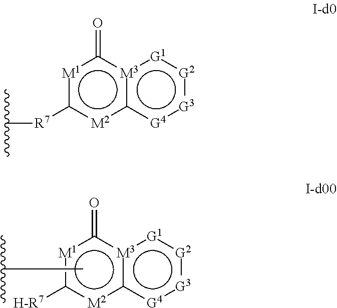

or a pharmaceutically acceptable salt thereof, wherein each of G¹, G², G³, G⁴, M¹, M², M³, and R⁷ is as defined in embodiments and classes and subclasses herein. For example, in certain embodiments, PIK2 is a PI3K binding moiety of formula I-d0 or I-d00, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-BM or H;
R⁸ is -L⁸-R^{8A};
R^{G1} is -L^{G1}-R^{G1A};
R^{G2} is -L^{G2}-R^{G2A}.
R^{G3} is -L^{G3}-R^{G3A}.
R^{M1} is -L^{M1}-R^{M1A}.
R^{M2} is -L^{M2}-R^{M2A}.
each of L⁷, L⁸, L^{G1} L^{G2} L^{G3} L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3}), —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z⁷ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

In certain embodiments, PIK2 is an PI3K binding moiety of formula I-a0, 1-b0, I-c0, I-d0, or I-d00:

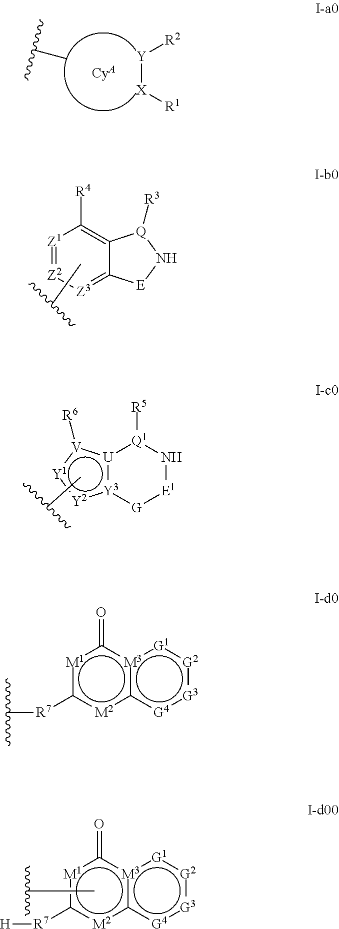

or a pharmaceutically acceptable salt thereof, wherein each of X, Y, Cy^{A}, R¹, R², E, Q, R³, R⁴, Z¹, Z², Z³, E¹, G, Q¹, R⁵, R⁶, U, V, Y¹, Y², Y³, G¹, G², G³, G⁴, M¹, M², M³, and R⁷ is as defined in embodiments and classes and subclasses herein.

In some embodiments, PIK2 is selected from the groups depicted in the compounds in Table 1, below.

In certain embodiments, the present disclosure provides a compound of formula I, wherein BM is PIK2, and PIK2 is a PI3K binding moiety of formula I-a0, thereby forming a compound of formula I-e:

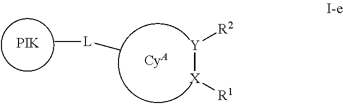

or a pharmaceutically acceptable salt thereof, wherein each of PIK, L, X, Y, Cy^{A}, R¹, and R² is as defined in embodiments and classes and subclasses herein.

In certain embodiments, the present disclosure provides a compound of formula I, wherein PIK is a PI3K binding moiety of formula I-a0, BM is PIK2, and PIK2 is a PI3K binding moiety of formula I-d0, thereby forming a compound of formula I-ee1:

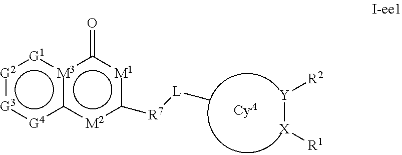

or a pharmaceutically acceptable salt thereof, wherein R⁷ is -L⁷-R^{7A} substituted with -L-, and each of L, L⁷, X, Y, Cy^{A}, G¹, G², G³, G⁴, M¹, M², M³, R¹, R², and R^{7A} is as defined in embodiments and classes and subclasses herein.

In certain embodiments, the present disclosure provides a compound of formula I, wherein BM is PIK2, and PIK2 is a PI3K binding moiety of formula I-b0, thereby forming a compound of formula I-f:

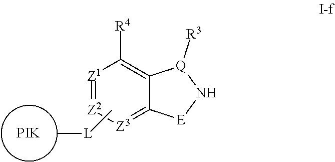

or a pharmaceutically acceptable salt thereof, wherein each of PIK, L, E, Q, R³, R⁴, Z¹, Z², and Z³ is as defined in embodiments and classes and subclasses herein.

In certain embodiments, the present disclosure provides a compound of formula I, wherein PIK is a PI3K binding moiety of formula I-b0, BM is PIK2, and PIK2 is a PI3K binding moiety of formula I-d0, thereby forming a compound of formula I-ff-1:

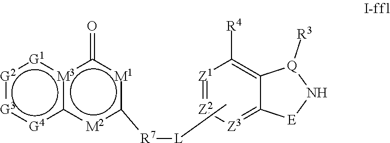

or a pharmaceutically acceptable salt thereof, wherein R⁷ is -L⁷-R^{7A} substituted with -L-, and each of L, L, G¹, G², G³, G⁴, M¹, M², M³, R^{7A}, E, Q, R³, R⁴, Z¹, Z², and Z³ is as defined in embodiments and classes and subclasses herein.

In certain embodiments, the present disclosure provides a compound of formula I, wherein BM is PIK2, and PIK2 is a PI3K binding moiety of formula I-c0, thereby forming a compound of formula I-g:

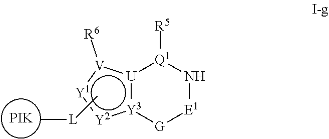

or a pharmaceutically acceptable salt thereof, wherein each of PIK, L, E¹, G, Q¹, R⁵, R⁶, U, V, Y¹, Y², and Y³ is as defined in embodiments and classes and subclasses herein.

In certain embodiments, the present disclosure provides a compound of formula I, wherein PIK is a PI3K binding moiety of formula I-c0, BM is PIK2, and PIK2 is a PI3K binding moiety of formula I-d0, thereby forming a compound of formula I-gg1:

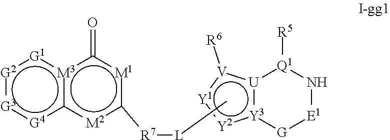

or a pharmaceutically acceptable salt thereof, wherein R⁷ is -L⁷-R^{7A} substituted with -L-, and each of L, L⁷, G¹, G², G³, G⁴, M¹, M², M³, R^{7A}, E¹, G, Q¹, R⁵, R⁶, U, V, Y¹, Y², and Y³ is as defined in embodiments and classes and subclasses herein.

In certain embodiments, the present disclosure provides a compound of formula I, wherein BM is PIK2, and PIK2 is a PI3K binding moiety of formula I-d0, thereby forming a compound of formula I-h:

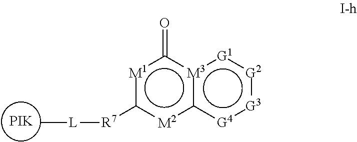

or a pharmaceutically acceptable salt thereof, wherein each of PIK, L, G¹, G², G³, G⁴, M¹, M², M³, and R⁷ is as defined in embodiments and classes and subclasses herein.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

TABLE 1
Representative Compounds of the Disclosure with Bioactivity Data.

| | | | | | MCF |
|---|---|---|---|---|---|
| | | | | ADP-Glo | 10A |
| Ex. | | | | IC50 | IC50 |
| No. | Structure | ¹H NMR | MS | (uM) | (uM) |
| I-1 | 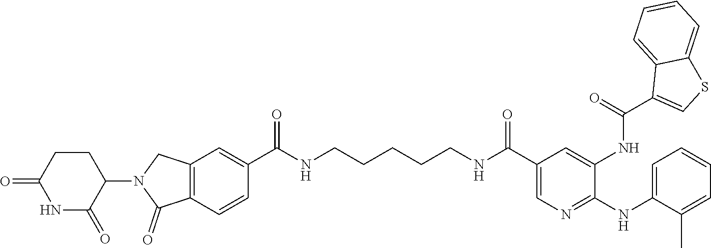 [SMILES: Cc1ccccc1Nc1ncc(C(=O)NCCCCCNC(=O)c2ccc3c(c2)CN(C2CCC(=O)NC2=O)C3=O)cc1NC(=O)c1csc2ccccc12] | 1H NMR (400 MHz, DMSO-d6) = 11.08-10.94 (m, 1H), 10.32-10.16 (m, 1H), 8.75-8.71 (m, 1H), 8.69- 8.62 (m, 1H), 8.55-8.47 (m, 2H), 8.44 (d, J = 1.9 Hz, 1H), 8.34 (s, 1H), 8.15 (s, 1H), 8.09 (dd, J = 1.3, 7.2 Hz, 1H), 8.04 (s, 1H), 7.98-7.93 (m, 1H), 7.81-7.77 (m, 1H), 7.52- 7.42 (m, 3H), 7.24-7.15 (m, 2H), 7.04 (s, 1H), 5.13 (dd, J = 5.1, 13.3 Hz, 1H), 4.56-4.34 (m, 2H), 3.30- 3.25 (m, 4H), 2.97-2.85 (m, 1H), 2.65-2.61 (m, 1H), 2.44-2.37 (m, 1H), 2.17 (s, 3H), 2.05-1.97 (m, 1H), 1.61-1.54 (m, 4H), 1.38 (d, J = 6.7 Hz, 2H) | 758.75 | B | D |
| I-2 | 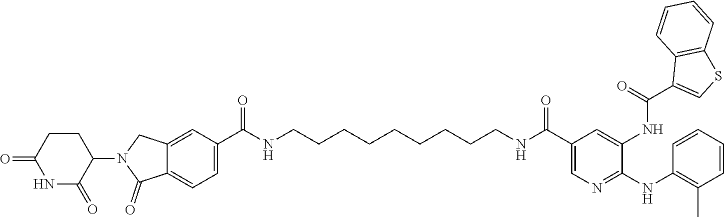 [SMILES: Cc1ccccc1Nc1ncc(C(=O)NCCCCCCCCCNC(=O)c2ccc3c(c2)CN(C2CCC(=O)NC2=O)C3=O)cc1NC(=O)c1csc2ccccc12] | 1H NMR (400 MHz, DMSO-d6) = 11.00 (s, 1H), 10.05 (s, 1H), 8.70 (s, 1H), 8.61 (s, 1H), 8.51-8.47 (m, 1H), 8.45 (d, J = 2.2 Hz, 1H), 8.34- 8.28 (m, 1H), 8.22-8.14 (m, 1H), 8.13-8.07 (m, 2H), 8.03 (s, 1H), 7.98-7.93 (m, 1H), 7.79 (d, J = 8.1 Hz, 1H), 7.53-7.42 (m, 3H), 7.24- 7.14 (m, 2H), 7.08-7.02 (m, 1H), 5.17-5.09 (m, 1H), 4.55-4.33 (m, 2H), 3.25 (dd, J = 5.8, 12.4 Hz, 4H), 2.91 (s, 1H), 2.65-2.56 (m, 2H), 2.44-2.38 (m, 1H), 2.17 (s, 3H), 2.06-1.98 (m, 1H), 1.52 (d, J = | 814.5 | C | D |
| | | 7.0 Hz, 4H), 1.30 (s, 9H). | | | |
| I-3 | 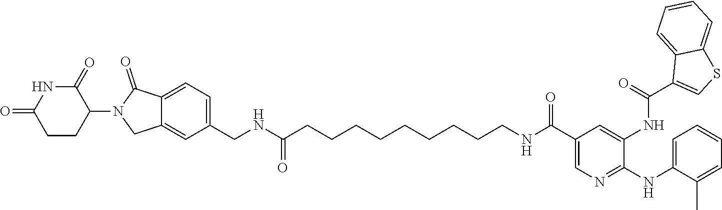 [SMILES: Cc1ccccc1Nc1ncc(C(=O)NCCCCCCCCCC(=O)NCc2ccc3c(c2)CN(C2CCC(=O)NC2=O)C3=O)cc1NC(=O)c1csc2ccccc12] | 1H NMR (400 MHz, DMSO-d6) = 11.13-10.83 (m, 1H), 10.57-10.26 (m, 1H), 8.75 (d, J = 1.2 Hz, 1H), 8.53 (d, J = 7.6 Hz, 2H), 8.44-8.40 (m, 2H), 8.32 (d, J = 5.4 Hz, 1H), 8.18 (s, 1H), 8.08 (d, J = 7.3 Hz, 1H), 7.66 (d, J = 7.8 Hz, 1H), 7.54-7.41 (m, 4H), 7.37 (d, J = 7.9 Hz, 1H), 7.24-7.13 (m, 2H), 7.07- 6.99 (m, 1H), 5.10 (dd, J = 5.1, 13.4 Hz, 1H), 4.48-4.24 (m, 4H), 3.22 (s, 2H), 2.97-2.84 (m, 1H), 2.62-2.58 (m, 2H), 2.42-2.35 (m, | 828.55 | B | D |
| | | 1H), 2.22-2.10 (m, 5H), 2.04-1.94 | | | |
| | | (m, 1H), 1.51 (d, J = 5.9 Hz, 4H), | | | |
| | | 1.26 (s, 9H). | | | |
| I-4 | 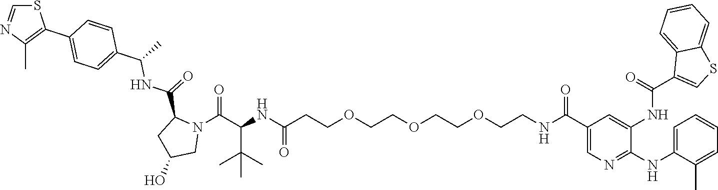 [SMILES: Cc1ccccc1Nc1ncc(C(=O)NCCOCCOCCOCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.04 (s, 1H), 8.98 (s, 1H), 8.70 (s, 1H), 8.51-8.35 (m, 4H), 8.21 (s, 1H), 8.13 (d, J = 2.1 Hz, 1H), 8.09 (dd, J = 1.2, 7.1 Hz, 1H), 7.85 (d, J = 9.4 Hz, 1H), 7.53-7.41 (m, 5H), 7.39-7.33 (m, 2H), 7.25-7.15 (m, 2H), 7.08-7.04 (m, 1H), 5.09 (d, J = 2.9 Hz, 1H), 4.91 ( t, J = 7.2 Hz, 1H), 4.52 (d, J = 9.4 Hz, 1H), 4.42 (t, J = 8.0 Hz, 1H), 4.30-4.24 (m, 1H), 3.63-3.36 (m, 17H), 2.45 (s, | 1034.4 | D | D |
| | | 3H), 2.17 (s, 3H), 2.12-1.91 (m, | | | |
| | | 1H), 1.85-1.75 (m, 1H), 1.37 (d, J = | | | |
| | | 7.0 Hz, 3H), 0.92 (s, 9H) | | | |
| I-5 | 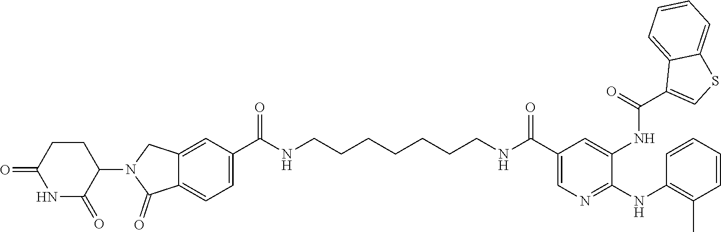 [SMILES: Cc1ccccc1Nc1ncc(C(=O)NCCCCCCCNC(=O)c2ccc3c(c2)CN(C2CCC(=O)NC2=O)C3=O)cc1NC(=O)c1csc2ccccc12] | 1H NMR (400 MHz, DMSO-d6) = 11.00 (s, 1H), 10.05 (s, 1H), 8.70 (s, 1H), 8.62 (t, J = 5.6 Hz, 1H), 8.49 (d, J = 7.0 Hz, 1H), 8.47-8.43 (m, 1H), 8.32 (s, 1H), 8.22-8.14 (m, 1H), 8.14-8.07 (m, 2H), 8.04 (s, 1H), 7.95 (d, J = 7.9 Hz, 1H), 7.79 (d, J = 7.9 Hz, 1H), 7.53-7.41 (m, 3H), 7.25-7.13 (m, 2H), 7.09- 7.02 (m, 1H), 5.17-5.09 (m, 1H), 4.55-4.34 (m, 2H), 3.29-3.24 (m, 3H), 2.90 (d, J = 13.1 Hz, 1H), 2.64-2.57 (m, 2H), 2.41 (dd, J = 4.4, 12.9 Hz, 1H), 2.17 (s, 3H), | 786.3 | B | D |
| | | 2.05-1.97 (m, 1H), 1.53 (d, J = 6.3 | | | |
| | | Hz, 4H), 1.33 (s, 6H). | | | |
| I-6 | 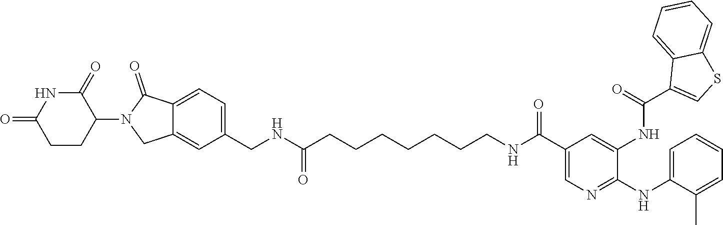 [SMILES: Cc1ccccc1Nc1ncc(C(=O)NCCCCCCCC(=O)NCc2ccc3c(c2)CN(C2CCC(=O)NC2=O)C3=O)cc1NC(=O)c1csc2ccccc12] | 1H NMR (400 MHz, DMSO-d6) = 10.97 (s, 1H), 10.05 (s, 1H), 8.70 (s, 1H), 8.51-8.47 (m, 1H), 8.44 (d, J = 2.1 Hz, 1H), 8.39 (t, J = 5.9 Hz, 1H), 8.32 ( t, J = 5.4 Hz, 1H), 8.21 ( s, 1H), 8.13-8.07 (m, 2H), 7.67 (d, J = 7.8 Hz, 1H), 7.51-7.42 (m, 4H), 7.37 (d, J = 8.1 Hz, 1H), 7.25-7.14 (m, 2H), 7.09-7.03 (m, 1H), 5.10 (dd, J = 5.0, 13.2 Hz, 1H), 4.47- 4.25 (m, 4H), 3.27-3.20 (m, 2H), 2.96-2.85 (m, 1H), 2.61 (s, 1H), 2.56 ( s, 1H), 2.42-2.35 (m, 1H), 2.19-2.16 (m, 3H), 2.15-2.13 (m, | 800.85 | B | D |
| | | 1H), 2.04-1.94 (m, 1H), 1.58-1.46 | | | |
| | | (m, 4H), 1.29 (s, 6H) | | | |
| 1-7 | 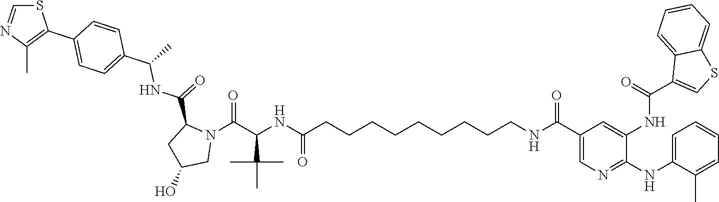 [SMILES: Cc1ccccc1Nc1ncc(C(=O)NCCCCCCCCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.06 (s, 1H), 8.98 (s, 1H), 8.70 (s, 1H), 8.52-8.47 (m, 1H), 8.45 (d, J = 2.1 Hz, 1H), 8.39-8.34 (m, 1H), 8.33-8.28 (m, 1H), 8.18 (s, 1H), 8.14-8.07 (m, 2H), 7.77 (d, J = 9.2 Hz, 1H), 7.50-7.41 (m, 5H), 7.40- 7.35 (m, 2H), 7.25-7.19 (m, 1H), 7.19-7.16 (m, 1H), 7.08-7.03 (m, 1H), 5.09 (d, J = 3.5 Hz, 1H), 4.92 (d, J = 7.0 Hz, 1H), 4.51 (d, J = 9.4 Hz, 1H), 4.45-4.37 (m, 1H), 4.38- 4.36 (m, 1H), 3.63-3.57 (m, 2H), | 999.7 | B | D |
| | | 3.27-3.21 (m, 2H), 2.45 (s, 3H), | | | |
| | | 2.29-2.21 (m, 1H), 2.19-2.14 (m, | | | |
| | | 3H), 2.14-2.07 (m, 1H), 2.07-1.96 | | | |
| | | (m, 1H), 1.83-1.72 (m, 1H), 1.55- | | | |
| | | 1.42 (m, 4H), 1.37 (d, J = 6.9 Hz, | | | |
| | | 3H), 1.32-1.20 (m, 10H), 0.93 (s, | | | |
| | | 9H) | | | |
| I-8 | 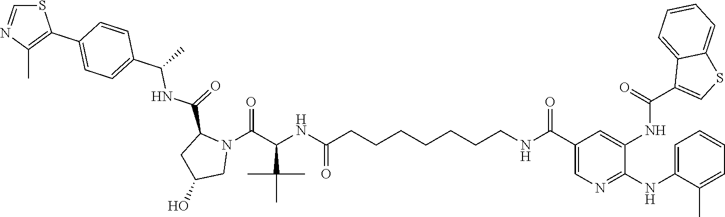 [SMILES: Cc1ccccc1Nc1ncc(C(=O)NCCCCCCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.05 (s, 1H), 8.98 (s, 1H), 8.72-8.69 (m, 1H), 8.49 (dd, J = 1.2, 7.0 Hz, 1H), 8.45 (d, J = 2.1 Hz, 1H), 8.39- 8.30 (m, 2H), 8.18 (s, 1H), 8.13- 8.07 (m, 2H), 7.78 (d, J = 9.2 Hz, 1H), 7.48-7.41 (m, 5H), 7.40-7.36 (m, 2H), 7.24-7.17 (m, 2H), 7.08- 7.02 (m, 1H), 5.11-5.06 (m, 1H), 4.91 (s, 1H), 4.51 (d, J = 9.4 Hz, 1H), 4.42 (s, 1H), 4.31-4.24 (m, 1H), 3.60 ( s, 2H), 3.24 ( d, J = 6.3 Hz, 2H), 2.45 (s, 3H), 2.23 (s, 1H), | 969.4 | B | D |
| | | 2.18 (s, 3H), 2.12 ( d, J = 7.6 Hz, | | | |
| | | 1H), 2.05-1.96 (m, 1H), 1.78 (s, | | | |
| | | 1H), 1.53-1.46 (m, 4H), 1.37 (d, J = | | | |
| | | 7.0 Hz, 3H), 1.28 (s, 6H), 0.92 (s, | | | |
| | | 9H) | | | |
| I-9 | 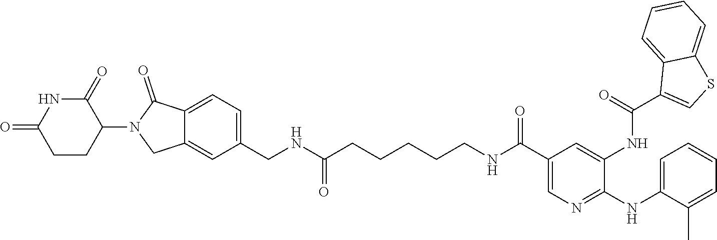 [SMILES: Cc1ccccc1Nc1ncc(C(=O)NCCCCCC(=O)NCc2ccc3c(c2)CN(C2CCC(=O)NC2=O)C3=O)cc1NC(=O)c1csc2ccccc12] | 1H NMR (400 MHz, DMSO-d6) = 10.97 (s, 1H), 10.05 (s, 1H), 8.70 (s, 1H), 8.45 (d, J = 2.1 Hz, 2H), 8.43- 8.37 (m, 1H), 8.35-8.29 (m, 1H), 8.18 (s, 1H), 8.13-8.07 (m, 2H), 7.67 (d, J = 7.8 Hz, 1H), 7.51-7.44 (m, 4H), 7.37 (d, J = 7.6 Hz, 1H), 7.25-7.14 (m, 2H), 7.09-7.01 (m, 1H), 5.10 (dd, J = 5.1, 13.3 Hz, 1H), 4.46-4.26 (m, 4H), 3.24 (d, J = 6.0 Hz, 2H), 2.97-2.85 (m, 1H), 2.60 (d, J = 2.8 Hz, 2H), 2.41-2.35 (m, 1H), 2.20-2.17 (m, 4H), 2.03-1.94 (m, 1H), 1.55 (td, J = 7.7, 15.8 Hz, 4H), 1.35-1.28 (m, 2H). | 772.8 | B | D |
| I-10 | 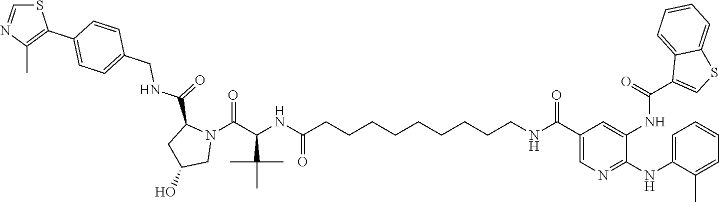 [SMILES: Cc1ccccc1Nc1ncc(C(=O)NCCCCCCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.06 (s, 1H), 8.98 (s, 1H), 8.70 (s, 1H), 8.55 (t, J = 6.0 Hz, 1H), 8.50 (d, J = 8.2 Hz, 1H), 8.44 (d, J = 2.1 Hz, 1H), 8.33 ( t, J = 5.7 Hz, 1H), 8.25 ( s, 1H), 8.18-8.03 (m, 2H), 7.83 (d, J = 9.4 Hz, 1H), 7.52-7.35 (m, 7H), 7.26-7.15 (m, 2H), 7.11- 7.02 (m, 1H), 4.54 (d, J = 9.4 Hz, 1H), 4.47-4.37 (m, 2H), 4.34 (s, 1H), 4.21 (dd, J = 5.4, 15.8 Hz, 1H), 3.75-3.55 (m, 2H), 3.27-3.07 (m, 2H), 2.47 (s, 3H), 2.28-2.22 | 985.35 | B | D |
| | | (m, 1H), 2.13 (s, 3H), 2.12-1.99 | | | |
| | | (m, 2H), 1.97-1.85 (m, 1H), 1.56- | | | |
| | | 1.41 (m, 4H), 1.35-1.16 (m, 11H), | | | |
| | | 0.98-0.87 (m, 9H) | | | |
| I-11 | 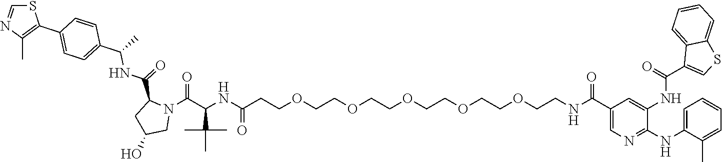 [SMILES: Cc1ccccc1Nc1ncc(C(=O)NCCOCCOCCOCCOCCOCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.04 (s, 1H), 8.98 (s, 1H), 8.70 (s, 1H), 8.55-8.35 (m, 4H), 8.21 (s, 1H), 8.16-8.06 (m, 2H), 7.86 (d, J = 9.4 Hz, 1H), 7.52-7.40 (m, 5H), 7.40-7.35 (m, 2H), 7.22 (d, J = 7.3 Hz, 1H), 7.18 (t, J = 7.8 Hz, 1H), 7.10-7.02 (m, 1H), 5.16-5.03 (m, 1H), 4.91 (t, J = 7.3 Hz, 1H), 4.52 (d, J = 9.4 Hz, 1H), 4.42 (t, J = 8.0 | 1122.6 | D | D |
| | | Hz, 1H), 4.27 ( s, 1H), 3.63-3.37 | | | |
| | | (m, 20H), 3.68-3.37 (m, 4H), 2.67 | | | |
| | | (td, J = 1.8, 3.7 Hz, 1H), 2.62-2.52 | | | |
| | | (m, 1H), 2.45 (s, 3H), 2.43-2.31 | | | |
| | | (m, 1H), 2.17 (s, 3H), 2.09-1.91 | | | |
| | | (m, 1H), 1.84-1.75 (m, 1H), 1.37 | | | |
| | | (d, J = 6.9 Hz, 3H), 0.93 (s, 9H) | | | |
| I-12 | 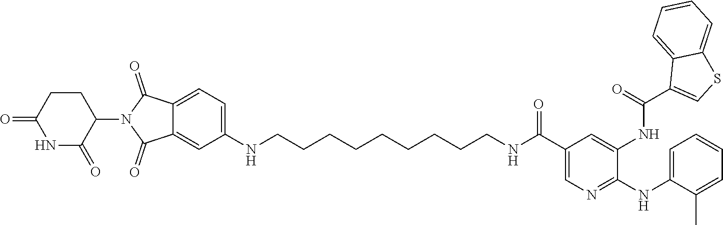 [SMILES: Cc1ccccc1Nc1ncc(C(=O)NCCCCCCCCCNc2ccc3c(c2)C(=O)N(C2CCC(=O)NC2=O)C3=O)cc1NC(=O)c1csc2ccccc12] | 1H NMR (400 MHz, DMSO-d6) = 11.08-11.03 (m, 1H), 10.05 (s, 1H), 8.70 (s, 1H), 8.51-8.47 (m, 1H), 8.45 (d, J = 2.2 Hz, 1H), 8.31 (t, J = 5.6 Hz, 1H), 8.18 (s, 1H), 8.13-8.07 (m, 2H), 7.55 (d, J = 8.4 Hz, 1H), 7.52-7.42 (m, 3H), 7.24-7.15 (m, 2H), 7.07 (s, 2H), 6.93 (d, J = 1.8 Hz, 2H), 6.83 (dd, J = 2.0, 8.4 Hz, 1H), 5.02 (dd, J = 5.4, 12.8 Hz, 1H), 3.24 (q, J = 6.6 Hz, 2H), 3.14 (q, J = 6.6 Hz, 2H), 2.93-2.81 (m, 1H), 2.59 (d, J = 2.2 Hz, 2H), 2.17 (s, 3H), 2.04-1.94 (m, 1H), 1.60-1.46 | 800.5 | B | D |
| | | (m, 4H), 1.36-1.26 (m, 9H). | | | |
| I-13 | 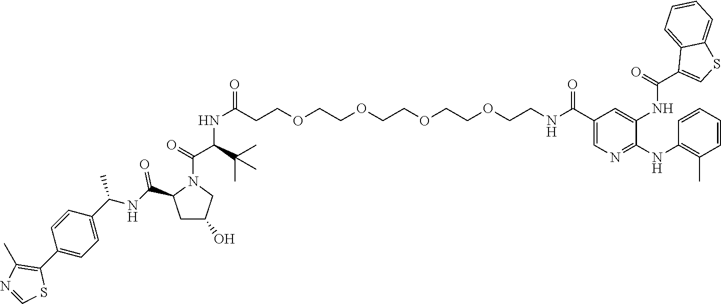 [SMILES: Cc1ccccc1Nc1ncc(C(=O)NCCOCCOCCOCCOCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.05 (s, 1H), 8.98 (s, 1H), 8.70 (s, 1H), 8.53-8.35 (m, 4H), 8.21 (s, 1H), 8.17-8.05 (m, 2H), 7.85 (d, J = 9.4 Hz, 1H), 7.52-7.40 (m, 5H), 7.40-7.35 (m, 2H), 7.22 (d, J = 7.5 Hz, 1H), 7.18 (t, J = 7.6 Hz, 1H), 7.10-7.02 (m, 1H), 5.10 (d, J = 3.5 Hz, 1H), 4.91 (t, J = 7.3 Hz, 1H), 4.52 (d, J = 9.2 Hz, 1H), 4.42 (t, J = 8.0 Hz, 1H), 4.27 ( s, 1H), 3.63-3.39 (m, 20H), 3.30-3.10 (m, 1H), 2.45 (s, 3H), 2.41-2.27 (m, 1H), 2.17 (s, 3H), 2.00 (d, J = 8.4 Hz, 1H), 1.83- 1.76 (m, 1H), 1.37 (d, J = 7.0 Hz, 3H), 0.93 (s, 9H). | 1077.8 | D | D |
| I-14 | 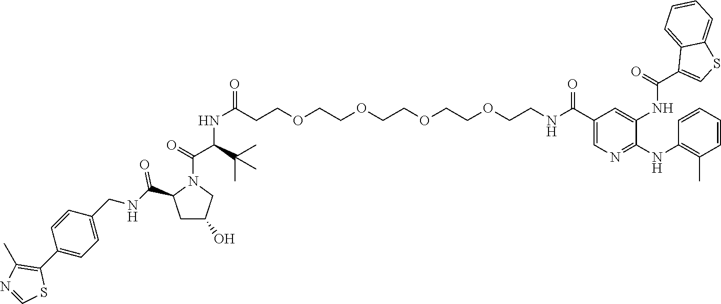 [SMILES: Cc1ccccc1Nc1ncc(C(=O)NCCOCCOCCOCCOCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.04 (s, 1H), 8.98 (s, 1H), 8.70 (s, 1H), 8.56 (s, 1H), 8.52-8.47 (m, 1H), 8.46 (d, J = 2.2 Hz, 1H), 8.42 (s, 1H), 8.21 (s, 1H), 8.15-8.11 (m, 1H), 8.11-8.06 (m, 1H), 7.91 (d, J = 9.4 Hz, 1H), 7.53-7.36 (m, 7H), 7.26-7.15 (m, 2H), 7.10-7.03 (m, 1H), 5.12 (d, J = 3.5 Hz, 1H), 4.55 (d, J = 9.4 Hz, 1H), 4.48-4.39 (m, 2H), 4.34 (s, 1H), 4.23 (d, J = 5.6 Hz, 1H), 3.69-3.38 (m, 20H), 2.58- 2.54 (m, 1H), 2.44 (s, 3H), 2.39- 2.33 (m, 1H), 2.17 (s, 3H), 2.06- 2.00 (m, 1H), 1.90 (s, 1H), 0.93 (s, 9H) | 1064.5 | C | |
| I-15 | 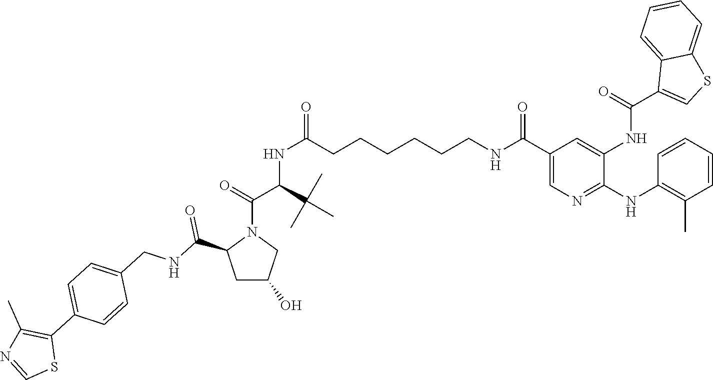 [SMILES: Cc1ccccc1Nc1ncc(C(=O)NCCCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.08 (s, 1H), 8.98 (s, 1H), 8.72- 8.69 (m, 1H), 8.56 (s, 1H), 8.50 (s, 1H), 8.43 (d, J = 2.1 Hz, 1H), 8.34 (s, 2H), 8.14 (d, J = 1.9 Hz, 1H), 8.09 (s, 1H), 7.88-7.81 (m, 1H), 7.52-7.36 (m, 7H), 7.27-7.16 (m, 2H), 7.07 (s, 1H), 4.54 (d, J = 9.4 Hz, 1H), 4.47-4.39 (m, 2H), 4.34 (s, 1H), 4.25-4.16 (m, 1H), 3.71- 3.62 (m, 2H), 3.30-3.20 (m, 2H), 2.44 (s, 3H), 2.30-2.21 (m, 2H), 2.18 (s, 3H), 2.16-2.10 (m, 1H), 2.06-1.98 (m, 1H), 1.89 (s, 1H), 1.49 (d, J = 7.0 Hz, 4H), 1.29 (d, J = 3.5 Hz, 4H), 0.93 (s, 9H) | 944.1 | B | |
| I-16 | 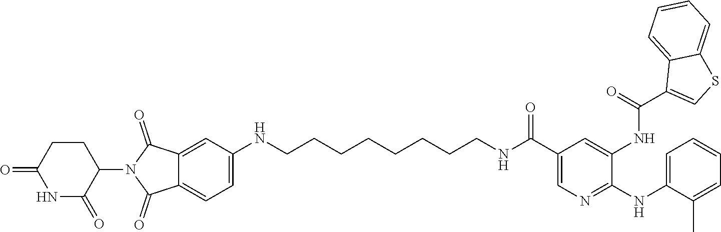 [SMILES: Cc1ccccc1Nc1ncc(C(=O)NCCCCCCCCNc2ccc3c(c2)C(=O)N(C2CCC(=O)NC2=O)C3=O)cc1NC(=O)c1csc2ccccc12] | 1H NMR (400 MHz, DMSO-d6) = 11.05 (s, 1H), 10.05 (s, 1H), 8.72- 8.67 (m, 1H), 8.51-8.45 (m, 1H), 8.45 (d, J = 2.2 Hz, 1H), 8.37-8.28 (m, 1H), 8.19 (s, 1H), 8.12-8.03 (m, 2H), 7.54 (d, J = 8.4 Hz, 1H), 7.51-7.40 (m, 3H), 7.24-7.13 (m, 2H), 7.11-7.04 (m, 2H), 6.93 (d, J = 1.8 Hz, 1H), 6.86-6.78 (m, 1H), 5.02 (dd, J = 5.4, 12.8 Hz, 1H), 3.24 (d, J = 6.5 Hz, 2H), 3.14 (d, J = 5.4 Hz, 2H), 2.94-2.80 (m, 1H), 2.59 (d, J = 2.3 Hz, 1H), 2.54 (s, 1H), 2.17 (s, 3H), 2.01-1.92 (m, 1H), 1.57 (s, | 786.25 | B | |
| | | 4H), 1.31 (s, 8H) | | | |
| I-17 | 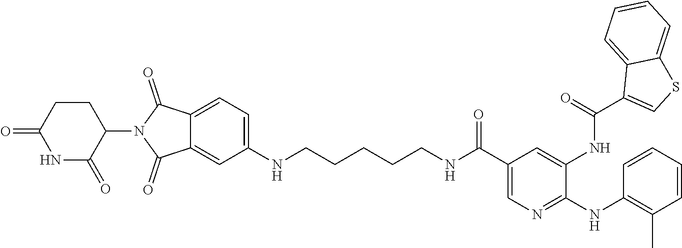 [SMILES: Cc1ccccc1Nc1ncc(C(=O)NCCCCCNc2ccc3c(c2)C(=O)N(C2CCC(=O)NC2=O)C3=O)cc1NC(=O)c1csc2ccccc12] | 1H NMR (400 MHz, DMSO-d6) = 11.03 (s, 1H), 10.03 (s, 1H), 8.71- 8.66 (m, 1H), 8.52-8.41 (m, 2H), 8.33 (s, 1H), 8.17 (s, 1H), 8.12-8.03 (m, 2H), 7.53 (d, J = 8.4 Hz, 1H), 7.49-7.37 (m, 3H), 7.24-7.13 (m, 2H), 7.09 (s, 1H), 7.04 (d, J = 1.2 Hz, 1H), 6.93 (d, J = 1.8 Hz, 1H), 6.83 (dd, J = 2.1, 8.4 Hz, 1H), 5.00 (dd, J = 5.5, 12.8 Hz, 1H), 3.26 (d, J = 6.0 Hz, 2H), 3.15 (d, J = 5.6 Hz, 2H), 2.90-2.79 (m, 1H), 2.56-2.51 (m, 2H), 2.16 (s, 3H), 2.02-1.94 (m, 1H), 1.67-1.51 (m, 4H), 1.40 (s, 2H) | 744.7 | A | C |
| I-18 | 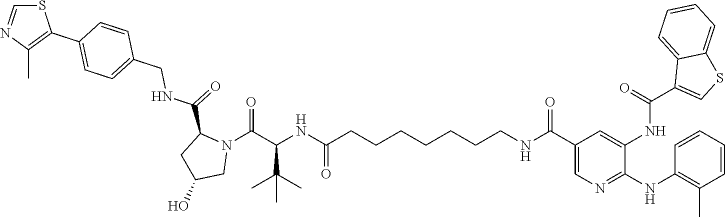 [SMILES: Cc1ccccc1Nc1ncc(C(=O)NCCCCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.05 (s, 1H), 8.98 (s, 1H), 8.71- 8.69 (m, 1H), 8.59-8.53 (m, 1H), 8.51-8.47 (m, 1H), 8.44 (d, J = 2.1 Hz, 1H), 8.32 (s, 1H), 8.18 (s, 1H), 8.12-8.06 (m, 2H), 7.86-7.81 (m, 1H), 7.51-7.38 (m, 7H), 7.25-7.16 (m, 2H), 7.09-7.03 (m, 1H), 5.11 (s, 1H), 4.54 (d, J = 9.4 Hz, 1H), 4.48- 4.38 (m, 2H), 4.34 (s, 1H), 4.26- 4.18 (m, 1H), 3.68-3.62 (m, 2H), 3.23 (d, J = 6.5 Hz, 2H), 2.44 (s, 3H), 2.24 (s, 1H), 2.18 (s, 3H), 2.15-2.09 (m, 1H), 2.05-1.99 (m, | 958.2 | B | |
| | | 1H), 1.95-1.85 (m, 1H), 1.55-1.46 | | | |
| | | (m, 4H), 1.28 (s, 6H), 0.93 (s, 9H) | | | |
| I-19 | 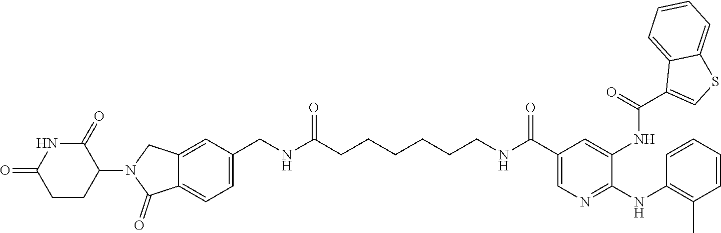 [SMILES: Cc1ccccc1Nc1ncc(C(=O)NCCCCCCC(=O)NCc2ccc3c(c2)CN(C2CCC(=O)NC2=O)C3=O)cc1NC(=O)c1csc2ccccc12] | 1H NMR (400 MHz, DMSO-d6) = 10.97 (s, 1H), 10.11-10.05 (m, 1H), 8.70 (s, 1H), 8.51-8.47 (m, 1H), 8.44 (d, J = 2.1 Hz, 1H), 8.42-8.38 (m, 1H), 8.36-8.31 (m, 1H), 8.27 (s, 1H), 8.13 (d, J = 1.8 Hz, 1H), 8.08 (s, 1H), 7.69-7.62 (m, 1H), 7.54-7.42 (m, 4H), 7.40-7.35 (m, 1H), 7.26-7.15 (m, 2H), 7.07 (d, J = 1.0 Hz, 1H), 5.09 (d, J = 8.1 Hz, 1H), 4.47-4.26 (m, 4H), 3.28- 3.21 (m, 2H), 2.95-2.83 (m, 1H), 2.58 (d, J = 18.3 Hz, 1H), 2.43- 2.33 (m, 1H), 2.18 (s, 3H), 2.15 (s, 2H), 2.01-1.94 (m, 1H), 1.60-1.46 | 786.35 | B | |
| | | (m, 4H), 1.34-1.26 (m, 4H) | | | |
| I-20 | 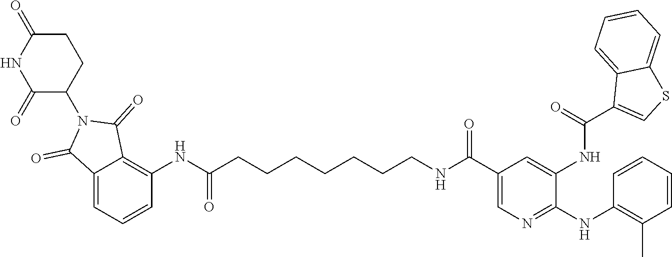 [SMILES: Cc1ccccc1Nc1ncc(C(=O)NCCCCCCCC(=O)Nc2cccc3c2C(=O)N(C2CCC(=O)NC2=O)C3=O)cc1NC(=O)c1csc2ccccc12] | 1H NMR (400 MHz, DMSO-d6) = 11.08 (s, 1H), 10.05 (s, 1H), 8.77- 8.63 (m, 1H), 8.53-8.47 (m, 1H), 8.45 (d, J = 2.2 Hz, 1H), 8.34-8.29 (m, 1H), 8.18 (s, 1H), 8.12-8.07 (m, 2H), 7.61-7.54 (m, 1H), 7.45 (d, J = 1.2 Hz, 3H), 7.25-7.15 (m, 2H), 7.08 (d, J = 8.5 Hz, 2H), 7.00 (d, J = 7.0 Hz, 1H), 6.52 (s, 1H), 5.04 (dd, J = 5.4, 12.8 Hz, 1H), 3.28-3.17 (m, 5H), 2.95-2.80 (m, 1H), 2.63-2.57 (m, 1H), 2.17 (s, 3H), 2.05-1.96 (m, 1H), 1.62-1.43 (m, 4H), 1.31 (s, 8H) | 786.85 | B | |
| I-21 | 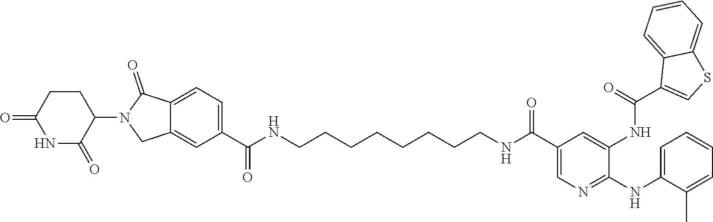 [SMILES: Cc1ccccc1Nc1ncc(C(=O)NCCCCCCCCNC(=O)c2ccc3c(c2)CN(C2CCC(=O)NC2=O)C3=O)cc1NC(=O)c1csc2ccccc12] | 1H NMR (400 MHz, DMSO-d6) = 11.00 (s, 1H), 10.05 (s, 1H), 8.70 (s, 1H), 8.62 (s, 1H), 8.52-8.45 (m, 1H), 8.45 (d, J = 2.1 Hz, 1H), 8.36- 8.26 (m, 1H), 8.18 (s, 1H), 8.14- 8.05 (m, 2H), 8.03 (s, 1H), 7.95 (dd, J = 1.0, 7.9 Hz, 1H), 7.79 (d, J = 7.9 Hz, 1H), 7.52-7.40 (m, 3H), 7.26-7.14 (m, 2H), 7.09-7.01 (m, 1H), 5.13 (dd, J = 5.1, 13.3 Hz, 1H), 4.56-4.32 (m, 2H), 3.25 (d, J = 6.6 Hz, 4H), 2.98-2.84 (m, 1H), 2.63-2.57 (m, 1H), 2.44-2.37 (m, 1H), 2.17 (s, 3H), 2.06-1.97 | 800.85 | B | |
| | | (m, 1H), 1.52 (d, J = 6.3 Hz, 4H), | | | |
| | | 1.31 (s, 8H) | | | |
| I-22 | 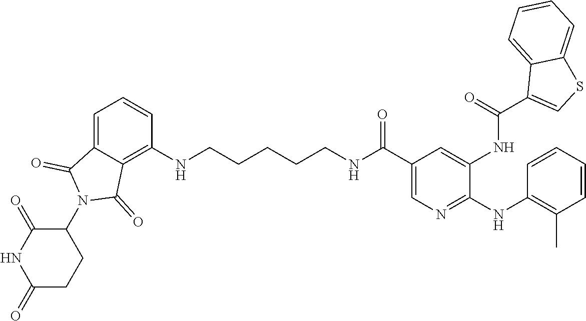 [SMILES: Cc1ccccc1Nc1ncc(C(=O)NCCCCCNc2cccc3c2C(=O)N(C2CCC(=O)NC2=O)C3=O)cc1NC(=O)c1csc2ccccc12] | 1H NMR (400 MHz, DMSO-d6) = 11.08 (s, 1H), 10.07 (s, 1H), 8.78- 8.65 (m, 1H), 8.51-8.47 (m, 1H), 8.44 (d, J = 2.1 Hz, 1H), 8.39-8.34 (m, 1H), 8.31-8.25 (m, 1H), 8.13 (d, J = 1.9 Hz, 1H), 8.11-8.07 (m, 1H), 7.60-7.55 (m, 1H), 7.52-7.44 (m, 3H), 7.23 (s, 2H), 7.09 (s, 2H), 7.03-6.99 (m, 1H), 6.55 (s, 1H), 3.29-3.18 (m, 5H), 2.87 (s, 1H), 2.55 (s, 2H), 2.18 (s, 3H), 2.05- 1.97 (m, 1H), 1.65-1.55 (m, 4H), 1.43-1.37 (m, 2H) | 744.75 | B | D |
| I-23 | 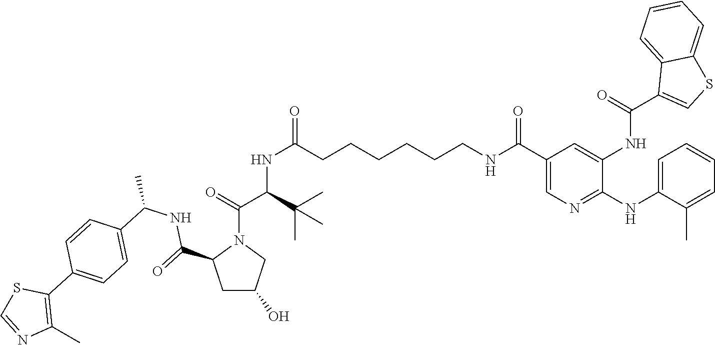 [SMILES: Cc1ccccc1Nc1ncc(C(=O)NCCCCCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.07 (s, 1H), 9.00-8.96 (m, 1H), 8.70 (s, 1H), 8.51-8.46 (m, 1H), 8.45 (d, J = 2.2 Hz, 1H), 8.39-8.35 (m, 1H), 8.34-8.28 (m, 1H), 8.20- 8.17 (m, 1H), 8.13-8.07 (m, 2H), 7.79 (d, J = 9.2 Hz, 1H), 7.52-7.41 (m, 5H), 7.39-7.32 (m, 2H), 7.24- 7.15 (m, 2H), 7.09-7.03 (m, 1H), 5.09 (d, J = 3.4 Hz, 1H), 4.95-4.87 (m, 1H), 4.54-4.49 (m, 1H), 4.42 (t, J = 8.0 Hz, 1H), 4.31-4.25 (m, 1H), 3.62-3.58 (m, 2H), 3.26-3.19 (m, 3H), 2.47-2.44 (m, 3H), 2.29- 2.20 (m, 1H), 2.18 (s, 3H), 2.11 (dd, J = 7.6, 14.2 Hz, 1H), 2.03- 1.97 (m, 1H), 1.83-1.75 (m, 1H), 1.55-1.44 (m, 4H), 1.37 (d, J = 7.0 Hz, 2H), 1.33-1.22 (m, 4H), 0.93 (s, 9H) | 958.25 | B | |
| I-24 | 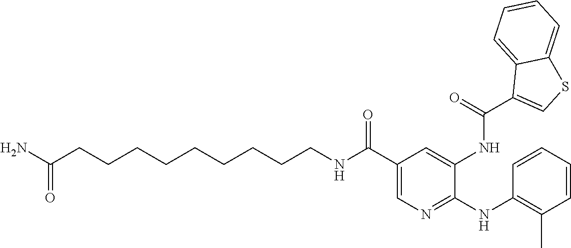 [SMILES: Cc1ccccc1Nc1ncc(C(=O)NCCCCCCCCCC(N)=O)cc1NC(=O)c1csc2ccccc12] | 1H NMR (400 MHz, DMSO-d6) = 10.09 (s, 1H), 8.76-8.69 (m, 1H), 8.52-8.48 (m, 1H), 8.46-8.43 (m, 1H), 8.35-8.29 (m, 1H), 8.20 (s, 1H), 8.14-8.07 (m, 2H), 7.53-7.42 (m, 3H), 7.24-7.14 (m, 3H), 7.08- 7.01 (m, 1H), 6.71-6.60 (m, 1H), 3.27-3.20 (m, 2H), 2.19-2.16 (m, 3H), 2.01 (t, J = 7.4 Hz, 2H), 1.54- 1.41 (m, 4H), 1.32-1.19 (m, 10H) | 572.5 | A | B |
| I-25 | 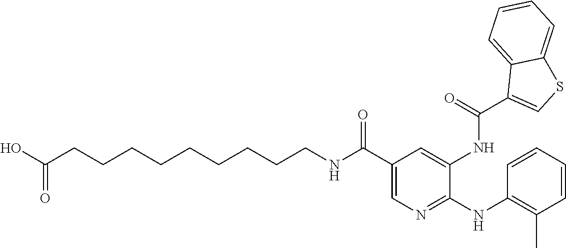 [SMILES: Cc1ccccc1Nc1ncc(C(=O)NCCCCCCCCCC(=O)O)cc1NC(=O)c1csc2ccccc12] | 1H NMR (400 MHz, DMSO-d6) = 10.10-10.06 (m, 1H), 8.70 (s, 1H), 8.52-8.46 (m, 1H), 8.43 (d, J = 2.2 Hz, 1H), 8.37-8.27 (m, 2H), 8.14 (d, J = 2.1 Hz, 1H), 8.11-8.06 (m, 1H), 7.51-7.42 (m, 3H), 7.26-7.15 (m, 2H), 7.11-7.05 (m, 1H), 3.29-3.20 (m, 2H), 2.21-2.15 (m, 5H), 1.55- 1.44 (m, 4H), 1.31-1.23 (m, 10H) | 573.45 | A | B |
| I-26 | 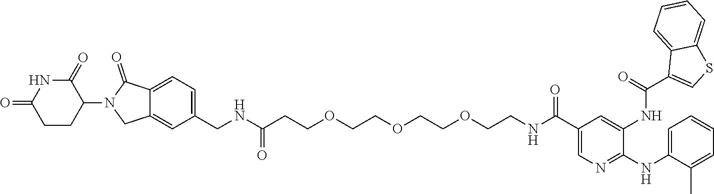 [SMILES: Cc1ccccc1Nc1ncc(C(=O)NCCOCCOCCOCCC(=O)NCc2ccc3c(c2)CN(C2CCC(=O)NC2=O)C3=O)cc1NC(=O)c1csc2ccccc12] | 1H NMR (400 MHz, DMSO-d6) = 11.05-10.89 (m, 1H), 10.14-10.01 (m, 1H), 8.81-8.64 (m, 1H), 8.52- 8.38 (m, 4H), 8.24 (s, 1H), 8.16- 8.12 (m, 1H), 8.09 (d, J = 7.3 Hz, 1H), 7.66 (d, J = 7.8 Hz, 1H), 7.52- 7.41 (m, 4H), 7.38 (d, J = 7.8 Hz, 1H), 7.25-7.15 (m, 2H), 7.09-7.03 (m, 1H), 5.14-5.06 (m, 1H), 4.44- 4.35 (m, 3H), 4.32-4.25 (m, 1H), 3.66-3.58 (m, 2H), 3.51-3.45 (m, 6H), 3.40 (q, J = 5.8 Hz, 2H), 3.01 | 862.25 | D | |
| | | (dt, J = 4.0, 6.5 Hz, 3H), 2.95-2.85 | | | |
| | | (m, 1H), 2.63-2.55 (m, 2H), 2.42- | | | |
| | | 2.34 (m, 3H), 2.17 (s, 3H), 2.04- | | | |
| | | 1.93 (m, 1H) | | | |
| I-27 | 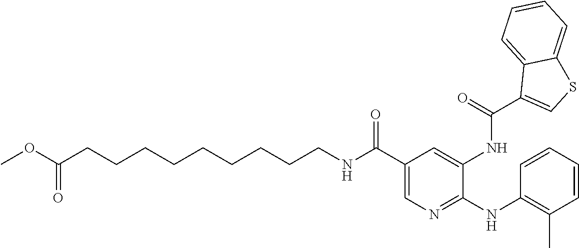 [SMILES: COC(=O)CCCCCCCCCNC(=O)c1cnc(Nc2ccccc2C)c(NC(=O)c2csc3ccccc23)c1] | 1H NMR (300 MHz, DMSO-d6) = 10.16 (s, 1H), 8.76-8.64 (m, 1H), 8.49 (dd, J = 2.0, 6.9 Hz, 2H), 8.41- 8.34 (m, 2H), 8.17 (d, J = 1.6 Hz, 1H), 8.12-8.06 (m, 1H), 7.53-7.40 (m, 3H), 7.29-7.17 (m, 2H), 7.15- 7.08 (m, 1H), 3.56 (s, 3H), 3.28- 3.20 (m, 2H), 2.30-2.24 (m, 2H), 2.19 (s, 3H), 1.50 (t, J = 6.3 Hz, 4H), 1.25 (s, 10H) | 587.45 | C | |
| I-28 | 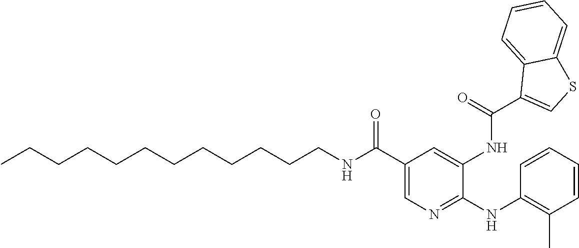 [SMILES: CCCCCCCCCCCCNC(=O)c1cnc(Nc2ccccc2C)c(NC(=O)c2csc3ccccc23)c1] | 1H NMR (400 MHz, DMSO-d6) = 10.17-10.10 (m, 1H), 8.71 (s, 1H), 8.53-8.47 (m, 1H), 8.44 (d, J = 2.1 Hz, 1H), 8.34-8.28 (m, 1H), 8.25- 8.19 (m, 1H), 8.14-8.06 (m, 2H), 7.53-7.42 (m, 3H), 7.25-7.15 (m, 2H), 7.09-7.02 (m, 1H), 3.26-3.19 (m, 2H), 2.18 (s, 3H), 1.55-1.46 (m, 2H), 1.28-1.21 (m, 18H), 0.87-0.82 (m, 3H) | 571.5 | D | D |
| I-29 | 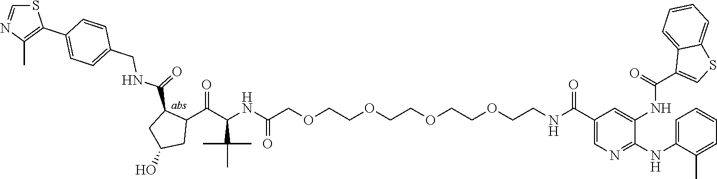 [SMILES: Cc1ccccc1Nc1ncc(C(=O)NCCOCCOCCOCCOCC(=O)N[C@H](C(=O)C2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.04 (s, 1H), 8.97 (s, 1H), 8.71- 8.65 (m, 1H), 8.63-8.56 (m, 1H), 8.46 (d, J = 2.2 Hz, 2H), 8.43-8.39 (m, 1H), 8.21 (s, 1H), 8.13 (d, J = 2.2 Hz, 1H), 8.11-8.07 (m, 1H), 7.39 (s, 8H), 7.26-7.14 (m, 2H), 7.11-7.01 (m, 1H), 5.15 (s, 1H), 4.56 (d, J = 9.7 Hz, 1H), 4.47-4.29 (m, 3H), 4.27 (d, J = 5.6 Hz, 1H), 3.95 (s, 2H), 3.71-3.44 (m, 15H), | 1050.55 | D | |
| | | 3.42-3.37 (m, 3H), 2.44 (s, 3H), | | | |
| | | 2.17 (s, 3H), 2.10-2.01 (m, 1H), | | | |
| | | 1.90 (s, 1H), 0.94 (s, 9H) | | | |
| I-30 | 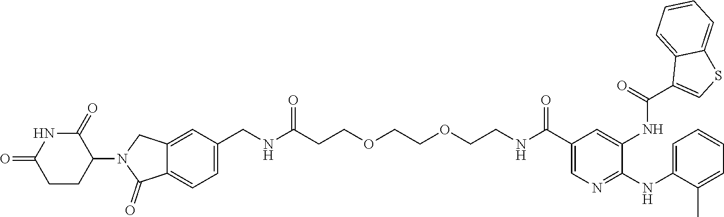 [SMILES: Cc1ccccc1Nc1ncc(C(=O)NCCOCCOCCC(=O)NCc2ccc3c(c2)CN(C2CCC(=O)NC2=O)C3=O)cc1NC(=O)c1csc2ccccc12] | 1H NMR (400 MHz, DMSO-d6) = 11.08-10.85 (m, 1H), 10.32-10.15 (m, 1H), 8.81-8.64 (m, 1H), 8.56- 8.47 (m, 4H), 8.43-8.29 (m, 2H), 8.20-8.12 (m, 1H), 8.11-8.05 (m, 1H), 7.69-7.60 (m, 1H), 7.55-7.40 (m, 3H), 7.40-7.32 (m, 1H), 7.26- 7.13 (m, 2H), 7.07-6.99 (m, 1H), 5.14-5.05 (m, 1H), 4.45-4.25 (m, 4H), 3.65 (t, J = 6.3 Hz, 2H), 3.56- 3.48 (m, 6H), 3.42-3.39 (m, 2H), 2.96-2.84 (m, 1H), 2.62-2.58 (m, 1H), 2.43-2.37 (m, 3H), 2.17 (s, 2H), 2.03-1.95 (m, 2H) | 818.3 | D | |
| I-31 | 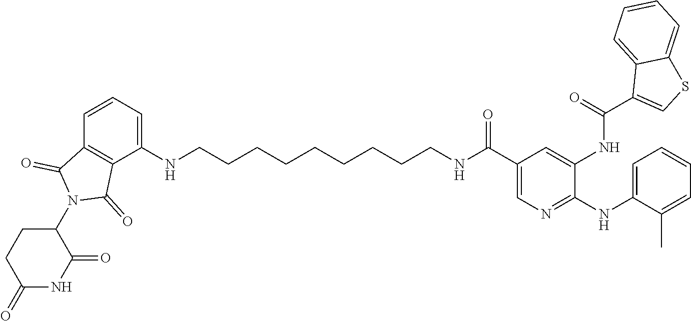 [SMILES: Cc1ccccc1Nc1ncc(C(=O)NCCCCCCCCCNc2cccc3c2C(=O)N(C2CCC(=O)NC2=O)C3=O)cc1NC(=O)c1csc2ccccc12] | 1H NMR (400 MHz, DMSO-d6) = 11.19-11.03 (m, 1H), 10.18-10.02 (m, 1H), 8.74-8.66 (m, 1H), 8.53- 8.48 (m, 1H), 8.44 (d, J = 2.1 Hz, 1H), 8.36-8.25 (m, 1H), 8.21 (s, 1H), 8.15-8.07 (m, 2H), 7.56 (dd, J = 7.2, 8.5 Hz, 1H), 7.52-7.42 (m, 3H), 7.25-7.13 (m, 2H), 7.10-7.05 (m, 2H), 7.01 (d, J = 6.9 Hz, 1H), 6.52 (t, J = 5.8 Hz, 1H), 5.04 (dd, J = 5.4, 12.8 Hz, 1H), 3.28-3.19 (m, 4H), 2.94-2.82 (m, 1H), 2.63-2.56 (m, 2H), 2.17 (s, 3H), 2.05-1.96 (m, 1H), 1.61-1.46 (m, 4H), 1.29 (s, 10H) | 800.85 | B | D |
| I-32 | 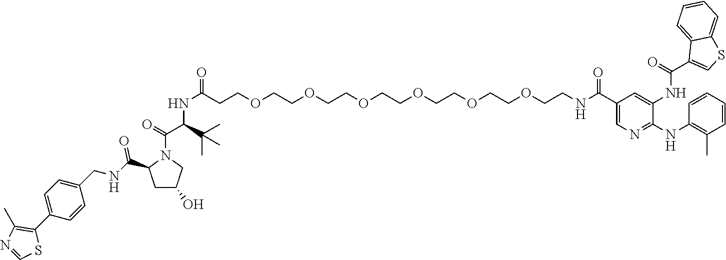 [SMILES: Cc1ccccc1Nc1ncc(C(=O)NCCOCCOCCOCCOCCOCCOCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.07-10.03 (m, 1H), 8.98 (s, 1H), 8.72-8.68 (m, 1H), 8.59-8.54 (m, 1H), 8.51-8.47 (m, 1H), 8.46 (d, J = 2.2 Hz, 1H), 8.44-8.39 (m, 1H), 8.25-8.19 (m, 1H), 8.14-8.12 (m, 1H), 8.11-8.06 (m, 1H), 7.91 (d, J = 9.4 Hz, 1H), 7.51-7.35 (m, 7H), 7.24-7.16 (m, 2H), 7.08-7.03 (m, 1H), 5.14-5.11 (m, 1H), 4.55 (d, J = 9.4 Hz, 1H), 4.46-4.39 (m, 2H), 4.35 (s, 1H), 4.25-4.18 (m, 1H), 3.69-3.56 (m, 4H), 3.55-3.46 (m, 22H), 3.44-3.38 (m, 2H), 2.57-2.53 (m, 1H), 2.47-2.42 (m, 3H), 2.38- 2.33 (m, 1H), 2.17 (s, 3H), 2.06- | 1152.65 | D | |
| | | 1.99 (m, 1H), 1.94-1.85 (m, 1H), | | | |
| | | 0.93 (s, 9H) | | | |
| I-33 | 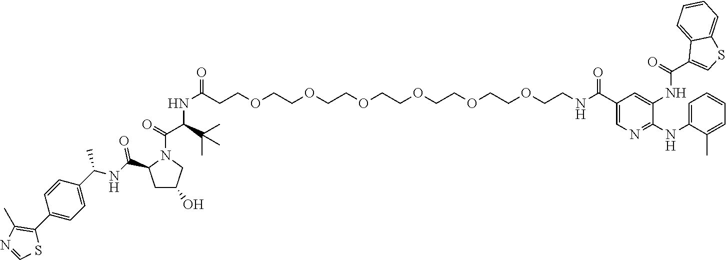 [SMILES: Cc1ccccc1Nc1ncc(C(=O)NCCOCCOCCOCCOCCOCCOCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.22-10.09 (m, 1H), 9.01-8.93 (m, 1H), 8.77-8.65 (m, 1H), 8.55-8.47 (m, 3H), 8.45 (s, 1H), 8.41 (d, J = 5.4 Hz, 1H), 8.39-8.35 (m, 1H), 8.30-8.23 (m, 1H), 8.17-8.12 (m, 1H), 8.10-8.06 (m, 1H), 7.86 (d, J = 9.4 Hz, 1H), 7.52-7.41 (m, 5H), 7.39-7.36 (m, 1H), 7.24-7.14 (m, 2H), 7.10-6.98 (m, 1H), 5.13-5.08 (m, 1H), 4.94-4.88 (m, 1H), 4.52 (d, J = 9.4 Hz, 1H), 4.46-4.40 (m, 1H), 4.31-4.25 (m, 1H), 3.64-3.55 (m, 4H), 3.55-3.51 (m, 7H), 3.50- 3.43 (m, 14H), 3.41 (dd, J = 6.4, 12.1 Hz, 4H), 2.45 (s, 3H), 2.18- | 1166.9 | D | |
| | | 2.16 (m, 1H), 2.20-2.15 (m, 2H), | | | |
| | | 2.05-1.97 (m, 1H), 1.78 (ddd, J = | | | |
| | | 4.4, 8.4, 12.6 Hz, 1H), 1.37 (d, J = | | | |
| | | 7.0 Hz, 3H), 0.93 (s, 9H) | | | |
| I-34 | 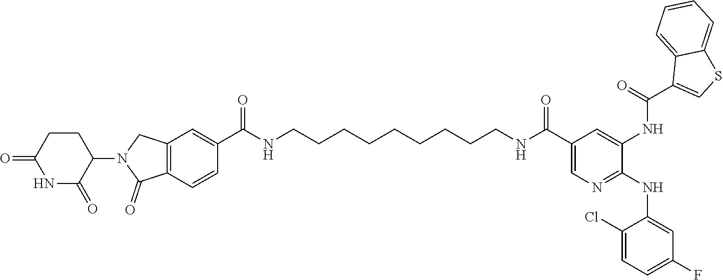 [SMILES: O=C1CCC(N2Cc3cc(C(=O)NCCCCCCCCCNC(=O)c4cnc(Nc5cc(F)ccc5Cl)c(NC(=O)c5csc6ccccc56)c4)ccc3C2=O)C(=O)N1] | 1H NMR (400 MHz, DMSO-d6) = 11.06-10.38 (m, 1H), 9.01-8.60 (m, 4H), 8.54 (s, 1H), 8.46-8.15 (m, 3H), 8.06-7.88 (m, 3H), 7.78 (d, J = 7.9 Hz, 1H), 7.51-7.34 (m, 3H), 6.82 (s, 1H), 5.19-5.06 (m, 1H), 4.55-4.24 (m, 2H), 3.29-3.23 (m, 4H), 2.97-2.82 (m, 1H), 2.61 (d, J = 2.5 Hz, 1H), 2.40 (dd, J = 4.5, 13.0 Hz, 1H), 2.04-1.97 (m, 1H), 1.53 (s, 4H), 1.31 (s, 10H) | 853.1 | C | |
| I-35 | 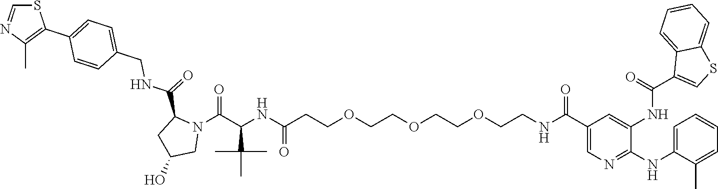 [SMILES: Cc1ccccc1Nc1ncc(C(=O)NCCOCCOCCOCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.04 (s, 1H), 8.98 (s, 1H), 8.71- 8.70 (m, 1H), 8.60-8.53 (m, 1H), 8.51-8.48 (m, 1H), 8.46 (d, J = 2.1 Hz, 1H), 8.42 (t, J = 5.5 Hz, 1H), 8.24-8.19 (m, 1H), 8.13 (d, J = 2.2 Hz, 1H), 8.10-8.07 (m, 1H), 7.93- 7.88 (m, 1H), 7.50-7.38 (m, 7H), 7.23-7.15 (m, 2H), 7.09-7.02 (m, 1H), 5.16-5.08 (m, 1H), 4.57-4.52 (m, 1H), 4.47-4.37 (m, 2H), 4.34 (s, 1H), 4.26-4.18 (m, 1H), 3.68-3.56 | 1020.4 | D | |
| | | (m, 4H), 3.53-3.45 (m, 10H), 3.44- | | | |
| | | 3.38 (m, 3H), 2.44-2.42 (m, 3H), | | | |
| | | 2.39-2.34 (m, 1H), 2.17 (s, 3H), | | | |
| | | 2.06-1.99 (m, 1H), 1.92-1.86 (m, | | | |
| | | 1H), 0.93 (s, 9H) | | | |
| I-36 | 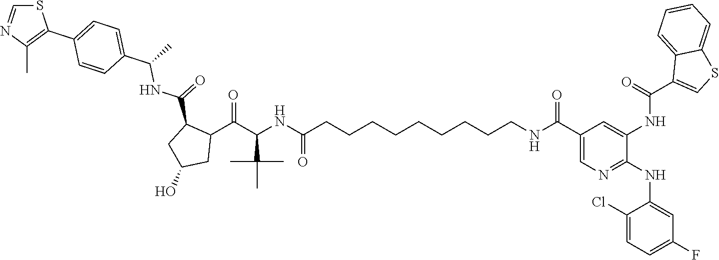 [SMILES: Cc1ncsc1-c1ccc([C@H](C)NC(=O)[C@@H]2C[C@@H](O)CC2C(=O)[C@@H](NC(=O)CCCCCCCCCNC(=O)c2cnc(Nc3cc(F)ccc3Cl)c(NC(=O)c3csc4ccccc34)c2)C(C)(C)C)cc1] | 1H NMR (400 MHz, DMSO-d6) = 10.57-10.47 (m, 1H), 9.02-8.90 (m, 1H), 8.74 (s, 1H), 8.70 (d, J = 2.1 Hz, 1H), 8.54-8.46 (m, 2H), 8.36 (d, J = 7.8 Hz, 1H), 8.32 (s, 1H), 8.21 (d, J = 2.1 Hz, 1H), 8.18 (dd, J = 3.1, 11.4 Hz, 1H), 8.12 (td, J = 0.7, 7.6 Hz, 1H), 7.77 (d, J = 9.2 Hz, 1H), 7.57-7.47 (m, 3H), 7.46-7.31 (m, 4H), 6.94-6.85 (m, 1H), 5.09 (d, J = 3.5 Hz, 1H), 4.96-4.86 (m, 1H), 4.54-4.48 (m, 1H), 4.44-4.37 (m, 1H), 4.27 (d, J = 2.3 Hz, 1H), 3.65- 3.54 (m, 2H), 3.45-3.40 (m, 1H), 3.29-3.22 (m, 2H), 2.45 (s, 3H), 2.30-2.19 (m, 1H), 2.14-2.05 (m, | 1038.85 | B | D |
| | | 1H), 2.04-1.96 (m, 1H), 1.82-1.73 | | | |
| | | (m, 1H), 1.57-1.43 (m, 4H), 1.37 | | | |
| | | (d, J = 7.0 Hz, 3H), 1.34-1.22 (m, | | | |
| | | 9H), 0.93 (s, 9H) | | | |
| I-37 | 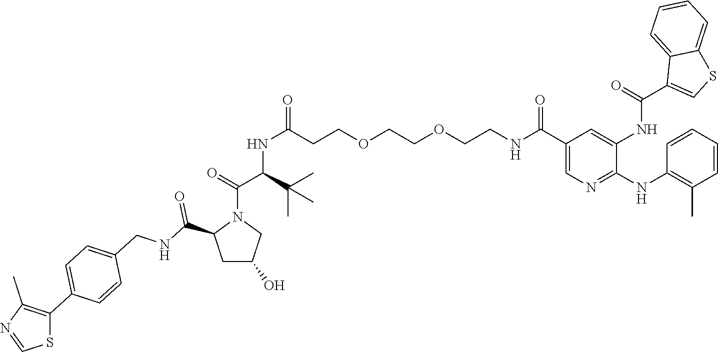 [SMILES: Cc1ccccc1Nc1ncc(C(=O)NCCOCCOCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.10 (s, 1H), 8.99-8.96 (m, 1H), 8.72-8.70 (m, 1H), 8.56 (t, J = 5.9 Hz, 1H), 8.52-8.47 (m, 2H), 8.45 (d, J = 2.2 Hz, 1H), 8.42 (t, J = 5.4 Hz, 1H), 8.27-8.21 (m, 1H), 8.16-8.12 (m, 1H), 8.11-8.07 (m, 1H), 7.94- 7.88 (m, 1H), 7.50-7.37 (m, 7H), 7.23-7.15 (m, 2H), 7.08-7.02 (m, 1H), 5.16-5.10 (m, 1H), 4.57-4.53 (m, 1H), 4.46-4.39 (m, 2H), 4.37- 4.31 (m, 1H), 4.26-4.18 (m, 1H), 3.67-3.57 (m, 4H), 3.55-3.46 (m, 8H), 2.45-2.43 (m, 3H), 2.40-2.35 (m, 1H), 2.19-2.15 (m, 3H), 2.06- 1.98 (m, 1H), 1.93-1.86 (m, 1H), 0.92 (s, 9H) | 975.35 | D | |
| I-38 | 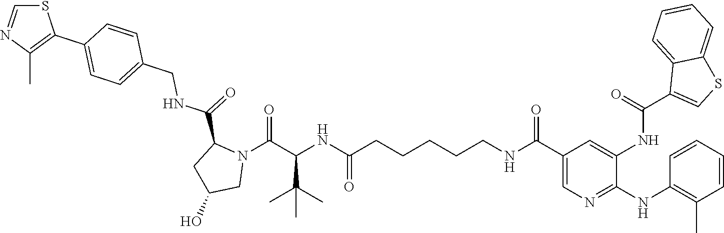 [SMILES: Cc1ccccc1Nc1ncc(C(=O)NCCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.05 (s, 1H), 8.99-8.95 (m, 1H), 8.71-8.68 (m, 1H), 8.59-8.53 (m, 1H), 8.51-8.42 (m, 2H), 8.33-8.27 (m, 1H), 8.18 (s, 1H), 8.13-8.07 (m, 2H), 7.88-7.81 (m, 1H), 7.51-7.35 (m, 7H), 7.23-7.15 (m, 2H), 7.08- 7.02 (m, 1H), 5.18-5.05 (m, 1H), 4.58-4.50 (m, 1H), 4.46-4.39 (m, 2H), 4.34 (s, 1H), 4.26-4.16 (m, 1H), 3.66-3.62 (m, 1H), 3.26-3.19 (m, 2H), 2.45-2.43 (m, 3H), 2.30- 2.22 (m, 1H), 2.17 (s, 3H), 2.15- 2.09 (m, 1H), 2.05-1.94 (m, 2H), 1.93-1.85 (m, 1H), 1.56-1.45 (m, | 927.35 | B | |
| | | 4H), 1.31-1.26 (m, 2H), 0.96-0.87 | | | |
| | | (m, 9H) | | | |
| I-39 | 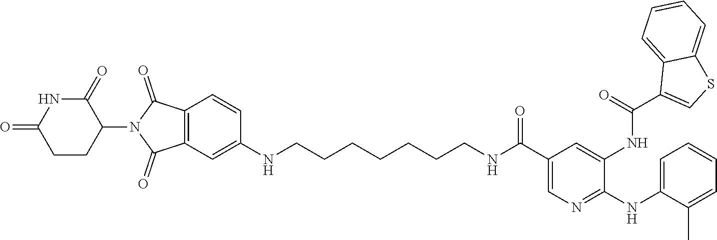 [SMILES: Cc1ccccc1Nc1ncc(C(=O)NCCCCCCCNc2ccc3c(c2)C(=O)N(C2CCC(=O)NC2=O)C3=O)cc1NC(=O)c1csc2ccccc12] | 1H NMR (400 MHz, DMSO-d6) = 11.12-11.01 (m, 1H), 10.22-10.08 (m, 1H), 8.78-8.63 (m, 1H), 8.54- 8.48 (m, 2H), 8.44 (d, J = 1.8 Hz, 1H), 8.35-8.30 (m, 1H), 8.27-8.21 (m, 1H), 8.13 (s, 1H), 8.11-8.07 (m, 1H), 7.58-7.53 (m, 1H), 7.50-7.43 (m, 2H), 7.24-7.14 (m, 2H), 7.12- 7.02 (m, 2H), 6.95-6.92 (m, 1H), 6.83 (dd, J = 2.0, 8.4 Hz, 1H), 5.02 (dd, J = 5.4, 12.8 Hz, 1H), 3.27- 3.21 (m, 2H), 3.18-3.12 (m, 2H), 2.92-2.81 (m, 1H), 2.63-2.57 (m, 2H), 2.18 (s, 3H), 2.03-1.93 (m, 1H), 1.63-1.48 (m, 4H), 1.35 (s, 6H) | 772.3 | B | D |
| I-40 | 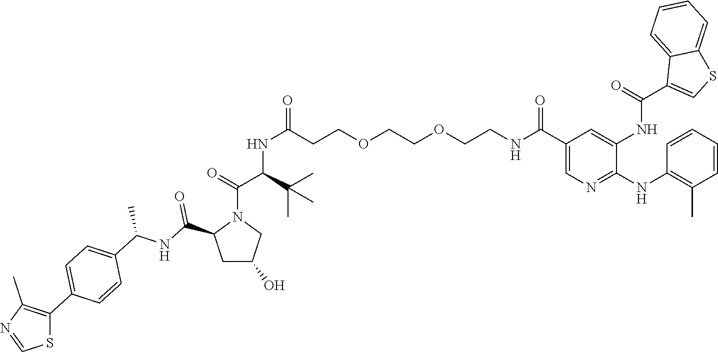 [SMILES: Cc1ccccc1Nc1ncc(C(=O)NCCOCCOCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.65-10.46 (m, 1H), 8.99-8.94 (m, 1H), 8.82 (s, 1H), 8.53-8.41 (m, 6H), 8.18 (d, J = 2.2 Hz, 1H), 8.10- 8.04 (m, 1H), 7.89-7.83 (m, 1H), 7.50-7.34 (m, 7H), 7.22-7.14 (m, 2H), 7.05 (dd, J = 1.2, 7.3 Hz, 1H), 4.93-4.86 (m, 1H), 4.54-4.49 (m, 1H), 4.44-4.40 (m, 1H), 4.31-4.24 (m, 1H), 3.63-3.57 (m, 4H), 3.54- 3.48 (m, 8H), 2.57-2.52 (m, 1H), 2.45 (s, 3H), 2.39-2.31 (m, 1H), 2.16 (s, 3H), 2.05-1.97 (m, 1H), 1.81-1.74 (m, 1H), 1.39-1.33 (m, 3H), 0.92 (s, 9H) | 989.45 | D | |
| I-41 | 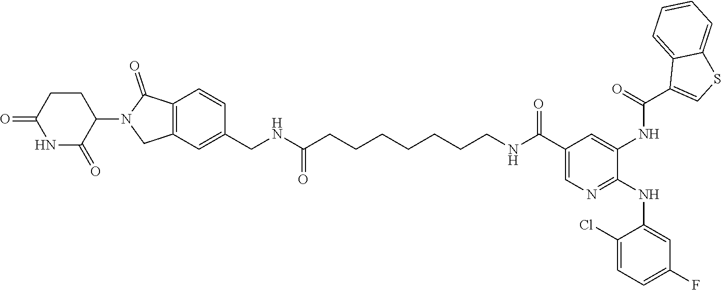 [SMILES: O=C(CCCCCCCNC(=O)c1cnc(Nc2cc(F)ccc2Cl)c(NC(=O)c2csc3ccccc23)c1)NCc1ccc2c(c1)CN(C1CCC(=O)NC1=O)C2=O] | 1H NMR (400 MHz, DMSO-d6) = 10.96 (br s, 1H), 10.55 (br s, 1H), 8.73-8.67 (m, 1H), 8.64-8.59 (m, 1H), 8.53 (dd, J = 3.2, 6.2 Hz, 1H), 8.49-8.44 (m, 2H), 8.39 (J = 5.8 Hz, 1H), 8.31-8.19 (m, 2H), 8.08 (d, J = 7.6 Hz, 1H), 7.65 (d, J = 7.9 Hz, 1H), 7.52-7.41 (m, 4H), 7.36 (d, J = 7.9 Hz, 1H), 6.90-6.82 (m, 1H), 5.11-5.05 (m, 1H), 4.45-4.23 (m, 4H), 3.25-3.21 (m, 2H), 2.94-2.82 (m, 1H), 2.62-2.58 (m, 1H), 2.55 (dd, J = 2.5, 4.4 Hz, 2H), 2.37 (dd, J = 4.7, 13.2 Hz, 1H), 2.17-2.10 (m, 2H), 2.01-1.94 (m, 1H), 1.56-1.47 (m, 4H), 1.29 (s, 6H) | 838.25 | B | |
| I-42 | 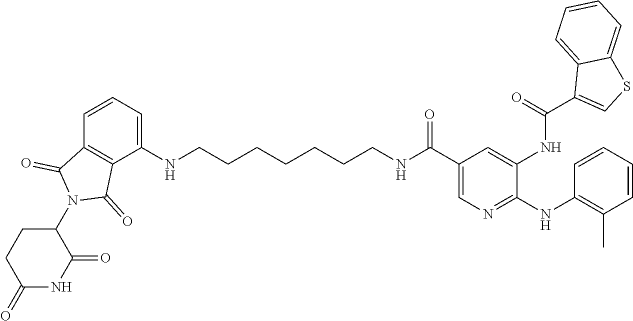 [SMILES: Cc1ccccc1Nc1ncc(C(=O)NCCCCCCCNc2cccc3c2C(=O)N(C2CCC(=O)NC2=O)C3=O)cc1NC(=O)c1csc2ccccc12] | 1H NMR (400 MHz, DMSO-d6) = 11.10 (br s, 1H), 10.24 (br s, 1H), 8.79-8.69 (m, 1H), 8.54-8.49 (m, 2H), 8.43 (d, J = 1.8 Hz, 1H), 8.34- 8.25 (m, 2H), 8.14 (s, 1H), 8.11- 8.07 (m, 1H), 7.56 (dd, J = 7.2, 8.5 Hz, 1H), 7.51-7.42 (m, 3H), 7.23- 7.14 (m, 2H), 7.10-6.99 (m, 3H), 6.53 (t, J = 5.8 Hz, 1H), 5.04 (dd, J = 5.5, 12.8 Hz, 1H), 3.28-3.19 (m, 4H), 2.94-2.82 (m, 1H), 2.59 (d, J = 2.6 Hz, 1H), 2.17 (s, 3H), 2.06-1.97 (m, 1H), 1.62-1.47 (m, 4H), 1.34 (s, 6H) | 772.75 | B | |
| I-43 | 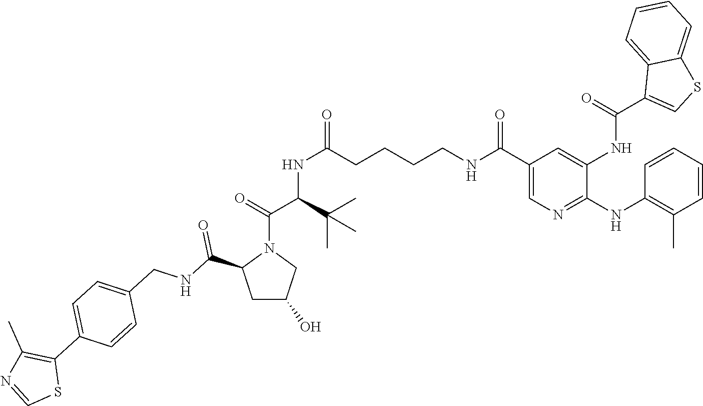 [SMILES: Cc1ccccc1Nc1ncc(C(=O)NCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.06 (s, 1H), 9.00-8.96 (m, 1H), 8.71-8.66 (m, 1H), 8.58-8.52 (m, 1H), 8.51-8.43 (m, 2H), 8.35 (t, J = 5.5 Hz, 1H), 8.18 (s, 1H), 8.12-8.06 (m, 2H), 7.91-7.84 (m, 1H), 7.52- 7.34 (m, 7H), 7.24-7.14 (m, 2H), 7.07-7.00 (m, 1H), 5.15-5.08 (m, 1H), 4.57-4.52 (m, 1H), 4.48-4.39 (m, 2H), 4.37-4.31 (m, 1H), 4.26- 4.17 (m, 1H), 3.71-3.60 (m, 2H), 3.28-3.21 (m, 2H), 2.44 (s, 3H), 2.34-2.25 (m, 1H), 2.19 (s, 1H), 2.17 (s, 3H), 2.07-1.98 (m, 1H), 1.93-1.85 (m, 1H), 1.57-1.44 (m, 4H), 0.93 (s, 9H) | 915.3 | C | |
| I-44 | 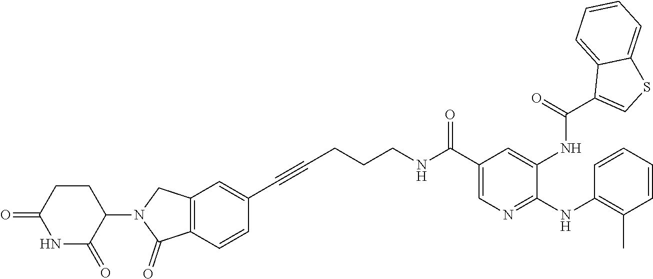 [SMILES: Cc1ccccc1Nc1ncc(C(=O)NCCCC#Cc2ccc3c(c2)CN(C2CCC(=O)NC2=O)C3=O)cc1NC(=O)c1csc2ccccc12] | 1H NMR (400 MHz, DMSO-d6) = 10.07 (s, 1H), 8.98 (s, 1H), 8.72- 8.67 (m, 1H), 8.52-8.47 (m, 1H), 8.45-8.43 (m, 1H), 8.39-8.30 (m, 2H), 8.25 (s, 1H), 8.14-8.07 (m, 2H), 7.79 (d, J = 9.4 Hz, 1H), 7.52- 7.42 (m, 5H), 7.39-7.35 (m, 2H), 7.25-7.16 (m, 2H), 7.10-7.04 (m, 1H), 4.91 (quin, J = 7.2 Hz, 1H), 4.51 (d, J = 9.2 Hz, 1H), 4.45-4.39 (m, 1H), 4.27 (d, J = 2.6 Hz, 1H), 3.64-3.57 (m, 2H), 3.28-3.20 (m, 3H), 2.45 (s, 3H), 2.29-2.21 (m, 1H), 2.18 (s, 3H), 2.16-2.09 (m, 1H), 2.01 (ddd, J = 2.4, 7.7, 12.6 Hz, 1H), 1.82-1.74 (m, 1H), 1.57- 1.45 (m, 4H), 1.37 (d, J = 7.0 Hz, | 711.65 | B | |
| | | 3H), 1.32-1.22 (m, 2H), 0.92 (s, | | | |
| | | 9H) | | | |
| I-45 | 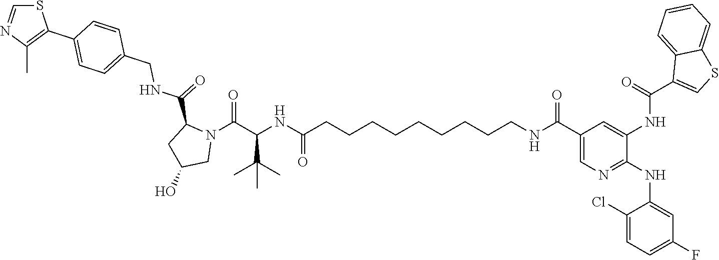 [SMILES: Cc1ncsc1-c1ccc(CNC(=O)[C@@H]2C[C@@H](O)CN2C(=O)[C@@H](NC(=O)CCCCCCCCCNC(=O)c2cnc(Nc3cc(F)ccc3Cl)c(NC(=O)c3csc4ccccc34)c2)C(C)(C)C)cc1] | 1H NMR (400 MHz, DMSO-d6) = 10.51 (s, 1H), 8.98 (s, 1H), 8.74 (s, 1H), 8.69 (d, J = 2.2 Hz, 1H), 8.57- 8.53 (m, 1H), 8.52-8.46 (m, 2H), 8.34-8.30 (m, 1H), 8.22-8.20 (m, 1H), 8.20-8.15 (m, 1H), 8.12 (td, J = 0.8, 7.7 Hz, 1H), 7.83 (d, J = 9.5 Hz, 1H), 7.54-7.45 (m, 3H), 7.43-7.36 (m, 4H), 6.93-6.85 (m, 1H), 5.11 (d, J = 3.4 Hz, 1H), 4.56- 4.51 (m, 1H), 4.46-4.39 (m, 2H), 4.36-4.32 (m, 1H), 4.25-4.18 (m, 1H), 3.69-3.60 (m, 2H), 3.29-3.24 (m, 2H), 2.44 (s, 3H), 2.29-2.21 (m, 1H), 2.14-1.99 (m, 2H), 1.90 (ddd, J = 4.7, 8.3, 13.0 Hz, 1H), 1.58-1.40 | 1025.4 | B | |
| | | (m, 4H), 1.32-1.23 (m, 10H), 0.93 | | | |
| | | (s, 9H) | | | |
| I-46 | 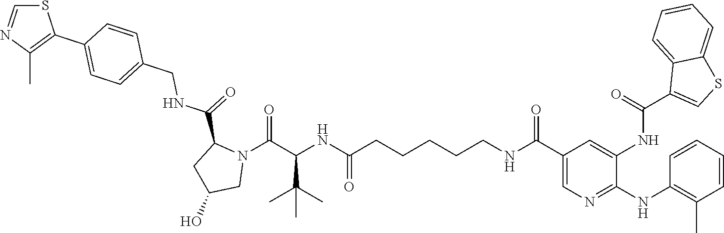 [SMILES: Cc1ccccc1Nc1ncc(C(=O)NCCCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.07 (s, 1H), 8.98 (s, 1H), 8.72- 8.67 (m, 1H), 8.52-8.47 (m, 1H), 8.45-8.43 (m, 1H), 8.39-8.30 (m, 2H), 8.25 (s, 1H), 8.14-8.07 (m, 2H), 7.79 (d, J = 9.4 Hz, 1H), 7.52- 7.42 (m, 5H), 7.39-7.35 (m, 2H), 7.25-7.16 (m, 2H), 7.10-7.04 (m, 1H), 4.91 (quin, J = 7.2 Hz, 1H), 4.51 (d, J = 9.2 Hz, 1H), 4.45-4.39 (m, 1H), 4.27 (d, J = 2.6 Hz, 1H), 3.64-3.57 (m, 2H), 3.28-3.20 (m, 3H), 2.45 (s, 3H), 2.29-2.21 (m, 1H), 2.18 (s, 3H), 2.16-2.09 (m, | 941.3 | B | |
| | | 1H), 2.01 (ddd, J = 2.4, 7.7, 12.6 | | | |
| | | Hz, 1H), 1.82-1.74 (m, 1H), 1.57- | | | |
| | | 1.45 (m, 4H), 1.37 (d, J = 7.0 Hz, | | | |
| | | 3H), 1.32-1.22 (m, 2H), 0.92 (s, | | | |
| | | 9H) | | | |
| I-47 | 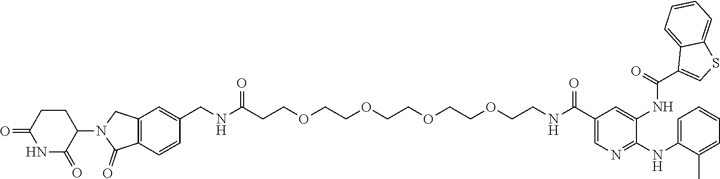 [SMILES: Cc1ccccc1Nc1ncc(C(=O)NCCOCCOCCOCCOCCC(=O)NCc2ccc3c(c2)CN(C2CCC(=O)NC2=O)C3=O)cc1NC(=O)c1csc2ccccc12] | 1H NMR (400 MHz, DMSO-d6) = 10.98 (s, 1H), 10.04 (s, 1H), 8.69 (s, 1H), 8.51-8.39 (m, 4H), 8.24- 8.20 (m, 1H), 8.13 (d, J = 2.1 Hz, 1H), 8.11-8.07 (m, 1H), 7.66 (d, J = 7.8 Hz, 1H), 7.52-7.41 (m, 4H), 7.38 (d, J = 7.9 Hz, 1H), 7.25-7.15 (m, 2H), 7.08-7.02 (m, 1H), 5.14- 5.06 (m, 1H), 4.46-4.36 (m, 3H), 4.33-4.25 (m, 1H), 3.68-3.59 (m, 2H), 3.55-3.44 (m, 14H), 3.40 (q, | 907.25 | D | |
| | | J = 5.8 Hz, 2H), 2.96-2.84 (m, 1H), | | | |
| | | 2.63-2.55 (m, 1H), 2.43-2.34 (m, | | | |
| | | 3H), 2.17 (s, 3H), 2.04-1.95 (m, | | | |
| | | 1H) | | | |
| I-48 | 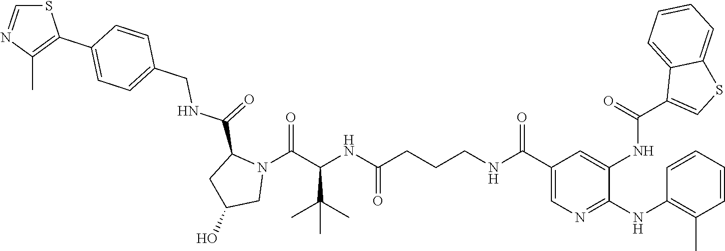 [SMILES: Cc1ccccc1Nc1ncc(C(=O)NCCCC(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.05 (s, 1H), 8.98 (s, 1H), 8.70 (s, 1H), 8.56 (t, J = 6.0 Hz, 1H), 8.51- 8.44 (m, 2H), 8.36 (t, J = 5.4 Hz, 1H), 8.19 (s, 1H), 8.13-8.07 (m, 2H), 7.97-7.92 (m, 1H), 7.52-7.34 (m, 7H), 7.23-7.14 (m, 2H), 7.08- 7.02 (m, 1H), 5.15-5.10 (m, 1H), 4.59-4.52 (m, 1H), 4.47-4.38 (m, 2H), 4.34 (d, J = 1.9 Hz, 1H), 4.25- 4.15 (m, 1H), 3.70-3.61 (m, 2H), 3.28-3.21 (m, 2H), 3.02 (s, 1H), 2.44 (s, 2H), 2.35-2.28 (m, 1H), 2.25-2.18 (m, 1H), 2.18 (s, 3H), 2.07-1.99 (m, 1H), 1.93-1.85 (m, 1H), 1.79-1.69 (m, 2H), 0.94 (s, | 899.3 | C | |
| | | 9H) | | | |
| I-49 | 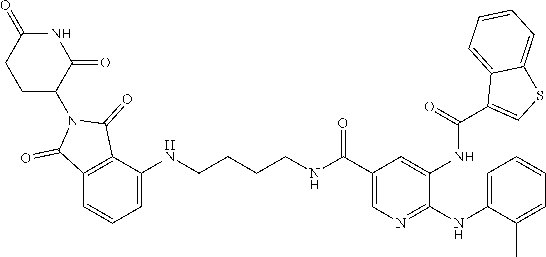 [SMILES: Cc1ccccc1Nc1ncc(C(=O)NCCCCNc2cccc3c2C(=O)N(C2CCC(=O)NC2=O)C3=O)cc1NC(=O)c1csc2ccccc12] | 1H NMR (400 MHz, DMSO-d6) = 11.08 (s, 1H), 10.07 (s, 1H), 8.70 (s, 1H), 8.51-8.44 (m, 2H), 8.38-8.32 (m, 1H), 8.20 (s, 1H), 8.12-8.08 (m, 2H), 7.57 (dd, J = 7.3, 8.4 Hz, 1H), 7.50-7.41 (m, 3H), 7.23-7.15 (m, 2H), 7.11 (s, 1H), 7.07-6.98 (m, 2H), 6.58 (t, J = 5.9 Hz, 1H), 5.04 (dd, J = 5.4, 12.8 Hz, 1H), 2.94- 2.82 (m, 1H), 2.63-2.54 (m, 3H), 2.18 (s, 3H), 2.05-1.95 (m, 1H), 1.68-1.54 (m, 4H) | 730.2 | B | |
| I-50 | 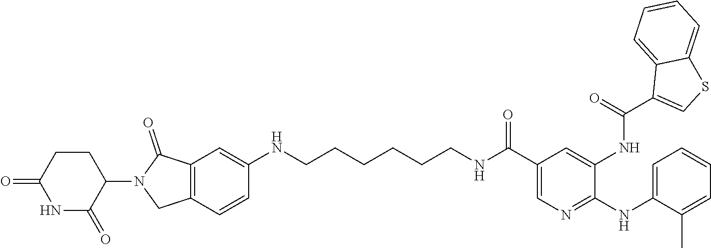 [SMILES: Cc1ccccc1Nc1ncc(C(=O)NCCCCCCNc2ccc3c(c2)C(=O)N(C2CCC(=O)NC2=O)C3)cc1NC(=O)c1csc2ccccc12] | 1H NMR (400 MHz, DMSO-d6) = 11.05 (s, 1H), 10.05 (s, 1H), 8.70 (s, 1H), 8.50-8.47 (m, 1H), 8.45 (d, J = 2.2 Hz, 1H), 8.33 (t, J = 5.6 Hz, 1H), 8.18 (s, 1H), 8.12-8.07 (m, 2H), 7.55 (d, J = 8.4 Hz, 1H), 7.52- 7.42 (m, 3H), 7.24-7.14 (m, 2H), 7.12-7.08 (m, 1H), 7.08-7.03 (m, 1H), 6.94 (d, J = 1.9 Hz, 1H), 6.84 (dd, J = 2.1, 8.4 Hz, 1H), 5.05-4.98 (m, 1H), 3.26 (q, J = 6.7 Hz, 2H), 3.19-3.11 (m, 2H), 2.92-2.81 (m, 1H), 2.61-2.54 (m, 2H), 2.17 (s, 3H), 2.02-1.94 (m, 1H), 1.62-1.50 (m, 4H), 1.45-1.32 (m, 4H) | 758.5 | B | |
| I-51 | 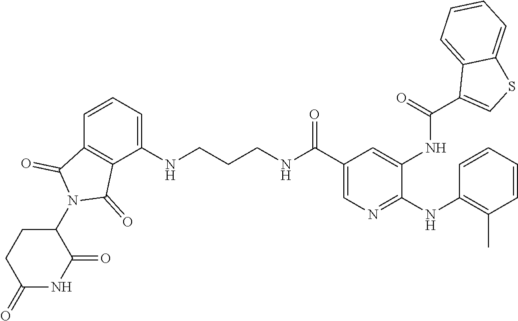 [SMILES: Cc1ccccc1Nc1ncc(C(=O)NCCCNc2cccc3c2C(=O)N(C2CCC(=O)NC2=O)C3=O)cc1NC(=O)c1csc2ccccc12] | 1H NMR (400 MHz, DMSO-d6) = 11.12-11.04 (m, 1H), 10.16-10.07 (m, 1H), 8.71 (s, 1H), 8.50 (d, J = 7.6 Hz, 1H), 8.47-8.40 (m, 2H), 8.27-8.21 (m, 1H), 8.14 (s, 1H), 8.09 (dd, J = 1.1, 7.0 Hz, 1H), 7.58 (dd, J = 7.3, 8.4 Hz, 1H), 7.51-7.40 (m, 3H), 7.24-7.15 (m, 2H), 7.12 (d, J = 8.7 Hz, 1H), 7.08-6.98 (m, 2H), 6.78-6.71 (m, 1H), 5.05 (dd, J = 5.4, 12.8 Hz, 1H), 3.43-3.37 (m, 3H), 2.93-2.81 (m, 1H), 2.62- 2.54 (m, 3H), 2.20-2.15 (m, 3H), 2.06-1.96 (m, 1H), 1.84-1.74 (m, 2H) | 716.65 | B | |
| I-52 | 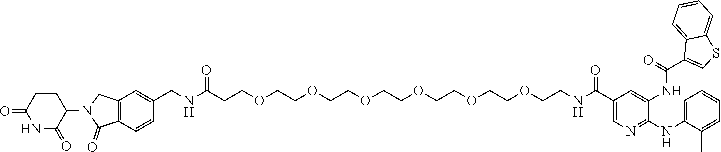 [SMILES: Cc1ccccc1Nc1ncc(C(=O)NCCOCCOCCOCCOCCOCCOCCC(=O)NCc2ccc3c(c2)CN(C2CCC(=O)NC2=O)C3=O)cc1NC(=O)c1csc2ccccc12] | 1H NMR (400 MHz, DMSO-d6) = 10.98 (s, 1H), 10.06 (s, 1H), 8.70 (s, 1H), 8.52-8.38 (m, 4H), 8.21 (s, 1H), 8.13 (d, J = 2.2 Hz, 1H), 8.09 (td, J = 0.7, 7.7 Hz, 1H), 7.68-7.64 (m, 1H), 7.52-7.35 (m, 5H), 7.25- 7.14 (m, 2H), 7.09-7.02 (m, 1H), 5.13-5.07 (m, 1H), 4.46-4.25 (m, 4H), 3.66-3.60 (m, 2H), 3.55-3.46 (m, 22H), 3.40 (d, J = 5.7 Hz, 2H), | 995.4 | E | |
| | | 2.91 (ddd, J = 5.4, 13.5, 17.4 Hz, | | | |
| | | 1H), 2.59 (td, J = 2.0, 15.3 Hz, | | | |
| | | 1H), 2.43-2.34 (m, 3H), 2.17 (s, | | | |
| | | 3H), 2.03-1.95 (m, 1H) | | | |
| I-53 | 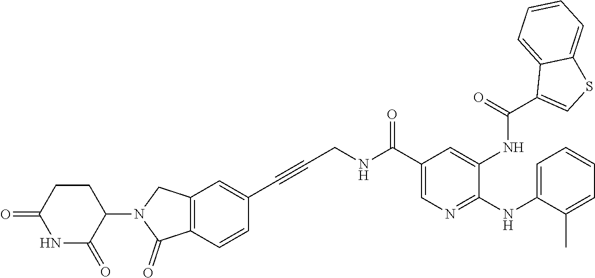 [SMILES: Cc1ccccc1Nc1ncc(C(=O)NCC#Cc2ccc3c(c2)CN(C2CCC(=O)NC2=O)C3=O)cc1NC(=O)c1csc2ccccc12] | 1H NMR (400 MHz, DMSO-d6) = 10.99 (s, 1H), 10.05 (s, 1H), 8.95 (t, J = 5.4 Hz, 1H), 8.71 (s, 1H), 8.54- 8.44 (m, 2H), 8.33 (s, 1H), 8.24- 8.19 (m, 1H), 8.14-8.06 (m, 1H), 7.75-7.66 (m, 2H), 7.60-7.37 (m, 4H), 7.28-7.15 (m, 2H), 7.12-7.02 (m, 1H), 5.04 (s, 1H), 4.51-4.28 (m, 4H), 2.98-2.85 (m, 1H), 2.63- 2.56 (m, 1H), 2.38 (br dd, J = 4.4, 13.1 Hz, 1H), 2.18 (s, 3H), 2.04- 1.96 (m, 1H) | 683.2 | A | A |
| I-54 | 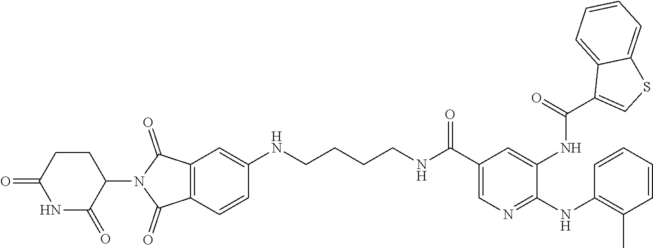 [SMILES: Cc1ccccc1Nc1ncc(C(=O)NCCCCNc2ccc3c(c2)C(=O)N(C2CCC(=O)NC2=O)C3=O)cc1NC(=O)c1csc2ccccc12] | 1H NMR (400 MHz, DMSO-d6) = 11.05 (s, 1H), 10.06 (s, 1H), 8.70 (s, 1H), 8.51-8.43 (m, 2H), 8.37 (t, J = 5.6 Hz, 1H), 8.20 (s, 1H), 8.13-8.08 (m, 2H), 7.55 (d, J = 8.4 Hz, 1H), 7.52-7.42 (m, 3H), 7.24-7.11 (m, 3H), 7.08-7.03 (m, 1H), 6.96 (d, J = 1.8 Hz, 1H), 6.85 (dd, J = 2.0, 8.4 Hz, 1H), 5.02 (dd, J = 5.4, 12.8 Hz, 1H), 3.20 (d, J = 5.3 Hz, 2H), 2.92-2.80 (m, 1H), 2.60-2.52 (m, 4H), 2.18 (s, 3H), 2.03-1.95 (m, 1H), 1.63 (s, 4H) | 730.5 | A | D |
| I-55 | 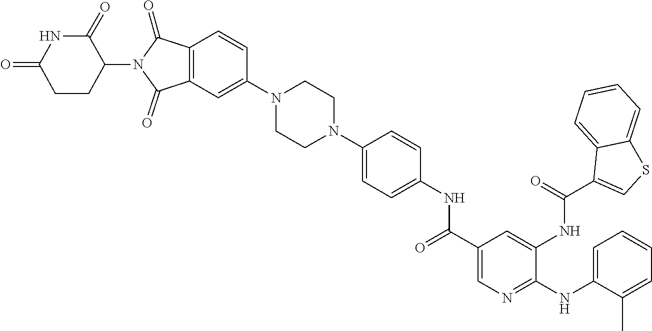 [SMILES: Cc1ccccc1Nc1ncc(C(=O)Nc2ccc(N3CCN(c4ccc5c(c4)C(=O)N(C4CCC(=O)NC4=O)C5=O)CC3)cc2)cc1NC(=O)c1csc2ccccc12] | 1H NMR (400 MHz, DMSO-d6) = 11.08 (s, 1H), 10.14 (s, 1H), 10.01 (s, 1H), 8.73 (s, 1H), 8.58-8.48 (m, 3H), 8.28 (d, J = 1.9 Hz, 1H), 8.10 (td, J = 1.0, 7.9 Hz, 1H), 7.72 (d, J = 8.5 Hz, 1H), 7.65 (d, J = 9.1 Hz, 2H), 7.54-7.39 (m, 4H), 7.34 (dd, J = 2.1, 8.7 Hz, 1H), 7.28-7.18 (m, 2H), 7.13 (d, J = 6.7 Hz, 1H), 7.08- 7.02 (m, 2H), 5.12-5.05 (m, 1H), 3.64 (s, 4H), 3.30 (s, 4H), 2.95-2.81 (m, 1H), 2.63-2.55 (m, 2H), 2.21 (s, 3H), 2.06-1.96 (m, 1H) | 819.5 | B | |
| I-56 | 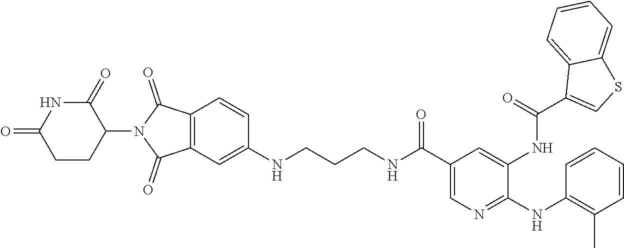 [SMILES: Cc1ccccc1Nc1ncc(C(=O)NCCCNc2ccc3c(c2)C(=O)N(C2CCC(=O)NC2=O)C3=O)cc1NC(=O)c1csc2ccccc12] | 1H NMR (400 MHz, DMSO-d6) = 11.05 (s, 1H), 10.05 (s, 1H), 8.70 (s, 1H), 8.51-8.44 (m, 2H), 8.41- 8.37 (m, 1H), 8.20 (s, 1H), 8.13- 8.06 (m, 2H), 7.57 (d, J = 8.4 Hz, 1H), 7.51-7.42 (m, 3H), 7.24-7.11 (m, 3H), 7.07-7.04 (m, 1H), 6.97 (d, J = 1.9 Hz, 1H), 6.87-6.84 (m, 1H), 5.02 (dd, J = 5.4, 12.8 Hz, 1H), 3.40-3.36 (m, 2H), 3.27-3.18 (m, 2H), 2.91-2.80 (m, 1H), 2.61- 2.57 (m, 2H), 2.18 (s, 3H), 2.04- 1.94 (m, 1H), 1.84 (quin, J = 6.8 Hz, 2H) | 716.25 | A | |
| I-57 | 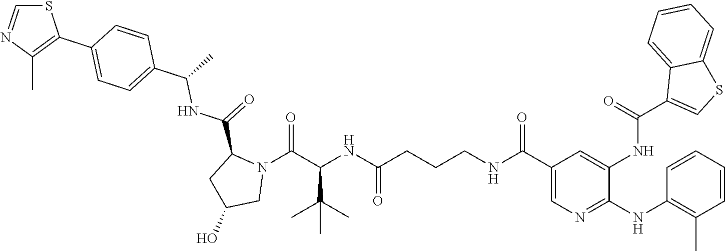 [SMILES: Cc1ccccc1Nc1ncc(C(=O)NCCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.06 (s, 1H), 8.98 (s, 1H), 8.70 (s, 1H), 8.51-8.45 (m, 2H), 8.39-8.33 (m, 2H), 8.21-8.18 (m, 1H), 8.14- 8.07 (m, 2H), 7.90 (d, J = 9.2 Hz, 1H), 7.52-7.31 (m, 7H), 7.25-7.14 (m, 2H), 7.09-7.03 (m, 1H), 5.12- 5.08 (m, 1H), 4.97-4.85 (m, 1H), 4.52 (d, J = 9.2 Hz, 1H), 4.45-4.40 (m, 1H), 4.27 (d, J = 3.1 Hz, 1H), 3.65-3.54 (m, 2H), 3.29-3.20 (m, 2H), 2.45 (s, 3H), 2.35-2.26 (m, 1H), 2.25-2.20 (m, 1H), 2.18 (s, 3H), 2.04-1.97 (m, 1H), 1.83-1.67 (m, 3H), 1.37 (d, J = 7.0 Hz, 3H), 0.94 (s, 9H) | 917.1 | C | |
| I-58 | 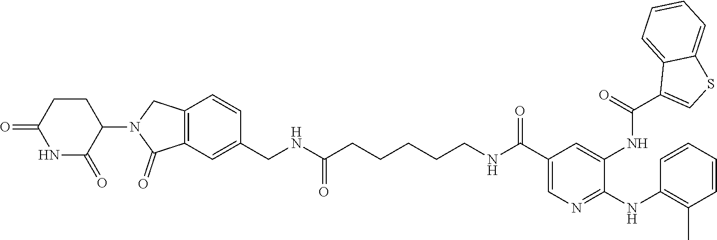 [SMILES: Cc1ccccc1Nc1ncc(C(=O)NCCCCCC(=O)NCc2ccc3c(c2)C(=O)N(C2CCC(=O)NC2=O)C3)cc1NC(=O)c1csc2ccccc12] | 1H NMR (400 MHz, DMSO-d6) = 10.98 (s, 1H), 10.06 (s, 1H), 8.70 (s, 1H), 8.51-8.31 (m, 4H), 8.21 (s, 1H), 8.14-8.06 (m, 2H), 7.60 (s, 1H), 7.53-7.42 (m, 5H), 7.24-7.15 (m, 2H), 7.09-7.03 (m, 1H), 5.10 (dd, J = 5.0, 13.4 Hz, 1H), 4.43- 4.24 (m, 4H), 3.24 (d, J = 6.3 Hz, 2H), 2.95-2.84 (m, 1H), 2.61-2.59 (m, 1H), 2.23-2.12 (m, 5H), 2.04- 1.94 (m, 2H), 1.62-1.49 (m, 4H), 1.36-1.28 (m, 2H) | 772.3 | B | |
| I-59 | 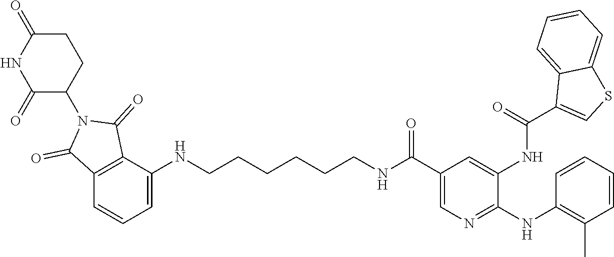 [SMILES: Cc1ccccc1Nc1ncc(C(=O)NCCCCCCNc2cccc3c2C(=O)N(C2CCC(=O)NC2=O)C3=O)cc1NC(=O)c1csc2ccccc12] | 1H NMR (400 MHz, DMSO-d6) = 11.08 (s, 1H), 10.05 (s, 1H), 8.69 (s, 1H), 8.51-8.41 (m, 2H), 8.34- 8.27 (m, 1H), 8.18 (s, 1H), 8.13- 8.07 (m, 2H), 7.56 (dd, J = 7.2, 8.5 Hz, 1H), 7.52-7.42 (m, 3H), 7.24- 7.14 (m, 2H), 7.11-6.98 (m, 3H), 6.54 (t, J = 5.8 Hz, 1H), 5.07-5.00 (m, 1H), 3.29-3.21 (m, 3H), 2.92- 2.81 (m, 1H), 2.62-2.55 (m, 2H), 2.17 (s, 3H), 2.06-1.95 (m, 2H), 1.64-1.47 (m, 4H), 1.42-1.31 (m, 4H) | 758.3 | C | |
| I-60 | 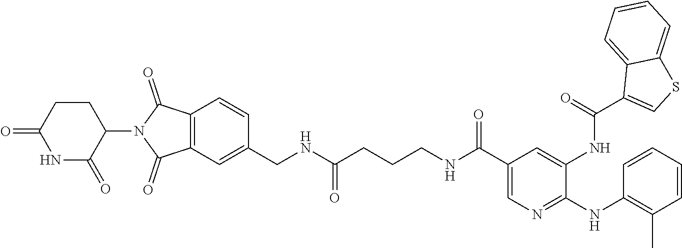 [SMILES: Cc1ccccc1Nc1ncc(C(=O)NCCCC(=O)NCc2ccc3c(c2)C(=O)N(C2CCC(=O)NC2=O)C3=O)cc1NC(=O)c1csc2ccccc12] | 1H NMR (400 MHz, DMSO-d6) = 10.97 (s, 1H), 10.05 (s, 1H), 8.70 (s, 1H), 8.51-8.44 (m, 3H), 8.37 (t, J = 5.5 Hz, 1H), 8.20 (s, 1H), 8.14- 8.06 (m, 2H), 7.66 (d, J = 7.8 Hz, 1H), 7.49-7.41 (m, 4H), 7.39 (d, J = 8.2 Hz, 1H), 7.23-7.14 (m, 2H), 7.08-6.98 (m, 1H), 5.12-5.07 (m, 1H), 4.46-4.24 (m, 4H), 3.27 (d, J = 6.0 Hz, 2H), 2.94-2.84 (m, 1H), 2.59 (d, J = 2.5 Hz, 1H), 2.42-2.34 (m, 1H), 2.23 (t, J = 7.5 Hz, 2H), 2.17 (s, 3H), 2.02-1.95 (m, 1H), 1.79 (quin, J = 7.3 Hz, 2H) | 744.75 | B | |
| I-61 | 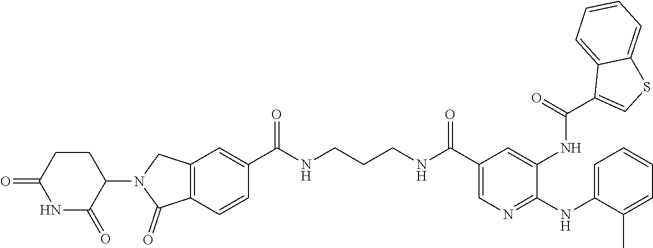 [SMILES: Cc1ccccc1Nc1ncc(C(=O)NCCCNC(=O)c2ccc3c(c2)CN(C2CCC(=O)NC2=O)C3=O)cc1NC(=O)c1csc2ccccc12] | 1H NMR (400 MHz, DMSO-d6) = 11.00 (s, 1H), 10.09 (s, 1H), 8.72- 8.67 (m, 2H), 8.52-8.47 (m, 1H), 8.46-8.40 (m, 2H), 8.35 (s, 1H), 8.16 (d, J = 1.9 Hz, 1H), 8.11-8.07 (m, 1H), 8.05 (s, 1H), 7.97 (d, J = 7.9 Hz, 1H), 7.81 (d, J = 7.9 Hz, 1H), 7.51-7.43 (m, 3H), 7.26-7.17 (m, 2H), 7.10-7.06 (m, 1H), 5.14 (dd, J = 5.1, 13.3 Hz, 1H), 4.55- 4.48 (m, 1H), 4.38 (d, J = 17.6 Hz, 1H), 3.38-3.32 (m, 4H), 2.96-2.87 (m, 1H), 2.60 (d, J = 17.8 Hz, 1H), 2.41 (br dd, J = 4.4, 13.2 Hz, 1H), 2.18 (s, 3H), 2.05-1.99 (m, 1H), 1.81 (quin, J = 6.8 Hz, 2H) | 730.75 | B | |
| I-62 | 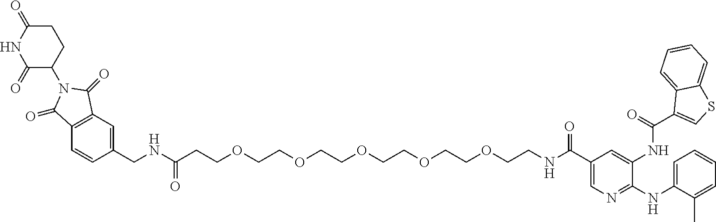 [SMILES: Cc1ccccc1Nc1ncc(C(=O)NCCOCCOCCOCCOCCOCCC(=O)NCc2ccc3c(c2)C(=O)N(C2CCC(=O)NC2=O)C3=O)cc1NC(=O)c1csc2ccccc12] | 1H NMR (400 MHz, DMSO-d6) = 10.98 (s, 1H), 10.04 (s, 1H), 8.70 (s, 1H), 8.51-8.38 (m, 4H), 8.21 (s, 1H), 8.15-8.06 (m, 2H), 7.66 (d, J = 7.8 Hz, 1H), 7.52-7.42 (m, 4H), 7.38 (d, J = 7.9 Hz, 1H), 7.24-7.15 (m, 2H), 7.09-7.04 (m, 1H), 5.10 (dd, J = 5.1, 13.4 Hz, 1H), 4.45- 4.24 (m, 4H), 3.63 (t, J = 6.3 Hz, 2H), 3.56-3.45 (m, 18H), 3.40 (q, J = 6.0 Hz, 2H), 3.06-2.84 (m, 1H), 2.64-2.56 (m, 1H), 2.43-2.35 (m, 3H), 2.17 (s, 3H), 2.05-1.92 (m, 1H) | 950.55 | D | |
| I-63 | 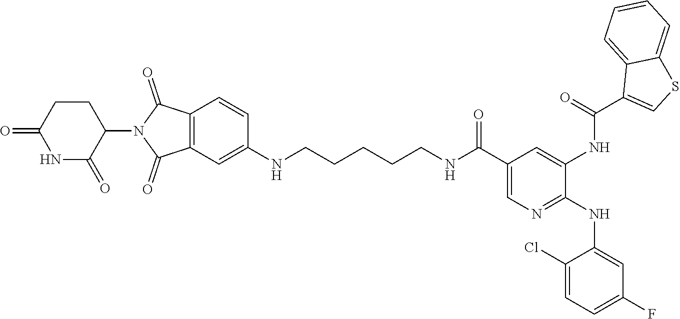 [SMILES: O=C1CCC(N2C(=O)c3ccc(NCCCCCNC(=O)c4cnc(Nc5cc(F)ccc5Cl)c(NC(=O)c5csc6ccccc56)c4)cc3C2=O)C(=O)N1] | 1H NMR (400 MHz, DMSO-d6) = 11.05 (s, 1H), 10.51 (s, 1H), 8.74 (s, 1H), 8.70 (d, J = 2.1 Hz, 1H), 8.56- 8.46 (m, 2H), 8.33 (s, 1H), 8.25-8.09 (m, 3H), 7.57-7.46 (m, 4H), 7.12 (t, J = 5.2 Hz, 1H), 6.98-6.82 (m, 3H), 5.02 (dd, J = 5.4, 12.8 Hz, 1H), 3.31-3.28 (m, 2H), 3.21-3.13 (m, 2H), 2.92-2.80 (m, 1H), 2.58 (d, J = 2.5 Hz, 2H), 2.03-1.93 (m, 1H), 1.67-1.54 (m, 4H), 1.49-1.38 (m, 2H) | 782.15 | B | |
| I-64 | 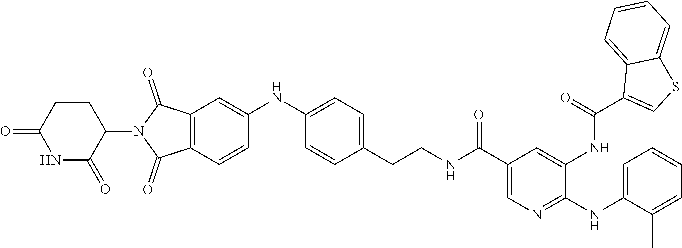 [SMILES: Cc1ccccc1Nc1ncc(C(=O)NCCc2ccc(Nc3ccc4c(c3)C(=O)N(C3CCC(=O)NC3=O)C4=O)cc2)cc1NC(=O)c1csc2ccccc12] | 1H NMR (300 MHz, DMSO-d6) = 11.09 (s, 1H), 10.05 (s, 1H), 9.16 (s, 1H), 8.70 (s, 1H), 8.53-8.43 (m, 3H), 8.27-8.19 (m, 1H), 8.18-8.05 (m, 2H), 7.71-7.63 (m, 1H), 7.55- 7.40 (m, 3H), 7.32-7.14 (m, 8H), 7.11-7.01 (m, 1H), 5.12-5.01 (m, 1H), 3.58-3.43 (m, 3H), 2.93-2.75 (m, 3H), 2.67-2.56 (m, 1H), 2.18 (s, 3H), 2.08-1.95 (m, 1H) | 778.7 | B | |
| I-65 | 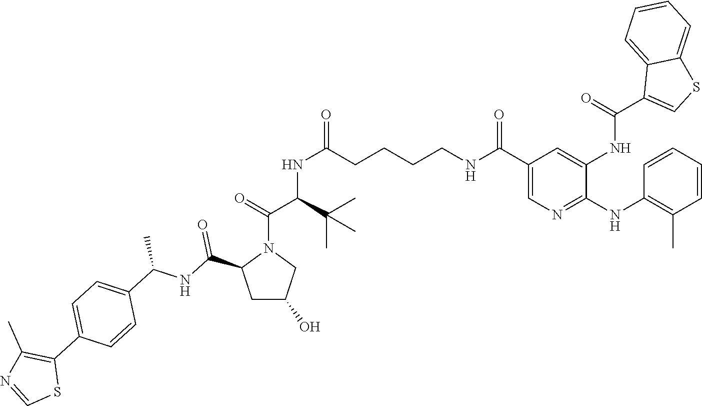 [SMILES: Cc1ccccc1Nc1ncc(C(=O)NCCCCC(=O)N[C@H](C(=O)N2C[C@H](O)C[C@H]2C(=O)N[C@@H](C)c2ccc(-c3scnc3C)cc2)C(C)(C)C)cc1NC(=O)c1csc2ccccc12] | 1H NMR (400 MHz, DMSO-d6) = 10.12 (s, 1H), 8.98 (s, 1H), 8.71 (s, 1H), 8.53-8.42 (m, 3H), 8.39-8.32 (m, 2H), 8.24-8.19 (m, 1H), 8.14- 8.07 (m, 2H), 7.81 (d, J = 9.2 Hz, 1H), 7.50-7.41 (m, 5H), 7.39-7.35 (m, 2H), 7.24-7.15 (m, 2H), 7.07- 7.02 (m, 1H), 5.10 (s, 1H), 4.96- 4.85 (m, 1H), 4.51 (d, J = 9.2 Hz, 1H), 4.44-4.38 (m, 1H), 4.27 (s, 1H), 3.60 (s, 2H), 3.24 (d, J = 5.7 Hz, 2H), 2.45 (s, 3H), 2.21-2.14 (m, 4H), 2.05-1.96 (m, 1H), 1.85- 1.75 (m, 1H), 1.57-1.45 (m, 4H), 1.40-1.34 (m, 3H), 0.93 (s, 9H) | 930.2 | C | |
| I-66 | 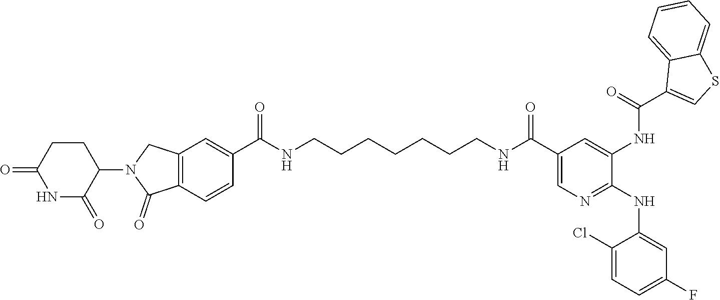 [SMILES: O=C1CCC(N2Cc3cc(C(=O)NCCCCCCCNC(=O)c4cnc(Nc5cc(F)ccc5Cl)c(NC(=O)c5csc6ccccc56)c4)ccc3C2=O)C(=O)N1] | 1H NMR (400 MHz, DMSO-d6) = 11.00 (s, 1H), 10.53 (s, 1H), 8.75 (s, 1H), 8.70 (d, J = 2.1 Hz, 1H), 8.63 (t, J = 5.6 Hz, 1H), 8.55-8.46 (m, 2H), 8.32 (s, 1H), 8.22-8.09 (m, 3H), 8.04 (s, 1H), 7.95 (dd, J = 1.2, 8.0 Hz, 1H), 7.79 (d, J = 7.9 Hz, 1H), 7.53-7.46 (m, 3H), 6.93-6.88 (m, 1H), 5.16-5.10 (m, 1H), 4.56- 4.32 (m, 2H), 3.29-3.24 (m, 4H), 2.97-2.85 (m, 1H), 2.64-2.56 (m, 2H), 2.04-1.94 (m, 1H), 1.55 (d, J = 5.6 Hz, 4H), 1.34 (s, 6H) | 824.15 | B | |
| I-67 | 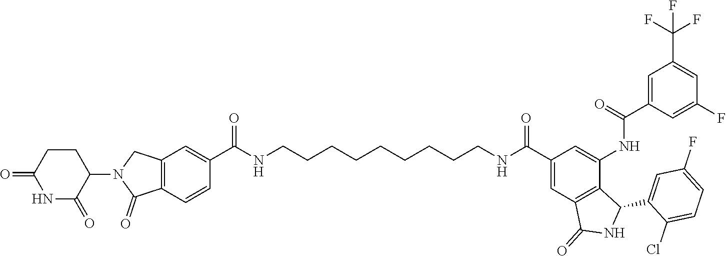 [SMILES: O=C1CCC(N2Cc3cc(C(=O)NCCCCCCCCCNC(=O)c4cc(NC(=O)c5cc(F)cc(C(F)(F)F)c5)c5c(c4)C(=O)N[C@H]5c4cc(F)ccc4Cl)ccc3C2=O)C(=O)N1] | 1H NMR (400 MHz, DMSO-d6) = 11.00 (s, 1H), 10.61 (s, 1H), 9.30- 9.20 (m, 1H), 8.78 (t, J = 5.5 Hz, 1H), 8.62 (t, J = 5.5 Hz, 1H), 8.22 (s, 1H), 8.07-7.88 (m, 4H), 7.84- 7.71 (m, 2H), 7.67 (s, 1H), 7.32 (dd, J = 5.1, 8.9 Hz, 1H), 7.14-7.03 (m, 1H), 6.13-5.92 (m, 1H), 5.18-5.08 (m, 1H), 4.55-4.47 (m, 1H), 4.42- 4.36 (m, 1H), 3.30-3.19 (m, 4H), 2.99-2.83 (m, 1H), 2.65-2.55 (m, 2H), 2.45-2.35 (m, 1H), 1.96 (d, J = 1.3 Hz, 1H), 1.54 (d, J = 5.6 Hz, 4H), 1.39-1.23 (m, 10H) | 922.3 | A | D |
| I-68 | 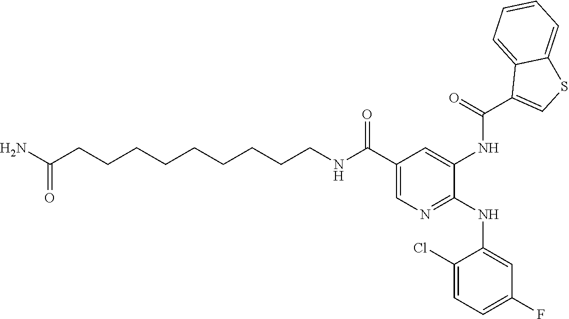 [SMILES: NC(=O)CCCCCCCCCNC(=O)c1cnc(Nc2cc(F)ccc2Cl)c(NC(=O)c2csc3ccccc23)c1] | 1H NMR (400 MHz, DMSO-d6) = 10.51 (s, 1H), 8.74 (s, 1H), 8.70 (d, J = 1.8 Hz, 1H), 8.49 (d, J = 6.7 Hz, 2H), 8.32 (s, 1H), 8.23-8.09 (m, 3H), 7.55-7.43 (m, 3H), 7.20 (s, 1H), 6.90 (dt, J = 2.9, 8.4 Hz, 1H), 6.66 (s, 1H), 3.29-3.23 (m, 2H), 2.04-1.97 (m, 2H), 1.56-1.43 (m, 4H), 1.34-1.22 (m, 10H) | 610.15 | B | |
| I-69 | 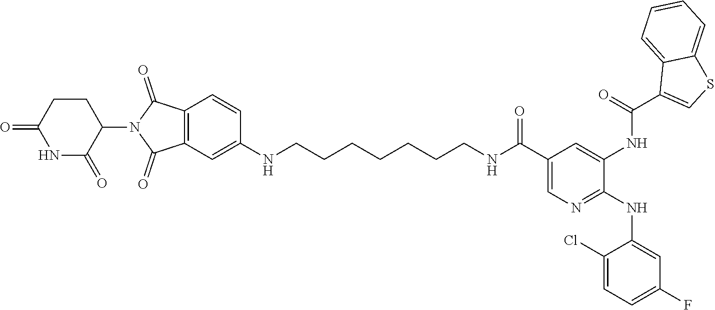 [SMILES: O=C1CCC(N2C(=O)c3ccc(NCCCCCCCNC(=O)c4cnc(Nc5cc(F)ccc5Cl)c(NC(=O)c5csc6ccccc56)c4)cc3C2=O)C(=O)N1] | 1H NMR (400 MHz, DMSO-d6) = 11.05 (s, 1H), 10.51 (s, 1H), 8.74 (s, 1H), 8.69 (d, J = 1.9 Hz, 1H), 8.53- 8.47 (m, 2H), 8.32 (s, 1H), 8.24- 8.09 (m, 3H), 7.44 (s, 4H), 7.10 (t, J = 5.3 Hz, 1H), 6.96-6.87 (m, 2H), 6.84 (dd, J = 2.1, 8.4 Hz, 1H), 5.02 (dd, J = 5.4, 12.8 Hz, 1H), 3.28 (d, J = 6.7 Hz, 2H), 3.18-3.12 (m, 2H), 2.92-2.81 (m, 1H), 2.63-2.57 (m, 2H), 2.03-1.94 (m, 1H), 1.63-1.50 (m, 4H), 1.36 (s, 6H) | 810.15 | B | |
| I-70 | 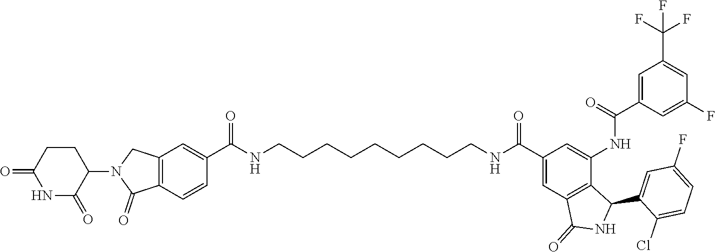 [SMILES: O=C1CCC(N2Cc3cc(C(=O)NCCCCCCCCCNC(=O)c4cc(NC(=O)c5cc(F)cc(C(F)(F)F)c5)c5c(c4)C(=O)N[C@@H]5c4cc(F)ccc4Cl)ccc3C2=O)C(=O)N1] | 1H NMR (400 MHz, DMSO-d6) = 11.00 (s, 1H), 10.61 (s, 1H), 9.25 (s, 1H), 8.78 (t, J = 5.4 Hz, 1H), 8.62 (t, J = 5.6 Hz, 1H), 8.22 (s, 1H), 8.06-7.88 (m, 4H), 7.82-7.65 (m, 3H), 7.32 (dd, J = 5.2, 8.9 Hz, 1H), 7.09 (dt, J = 3.0, 8.4 Hz, 1H), 6.12-5.94 (m, 1H), 5.13 (dd, J = 5.1, 13.3 Hz, 1H), 4.60-4.48 (m, 1H), 4.44-4.32 (m, 1H), 3.31-3.21 (m, 4H), 3.00-2.84 (m, 1H), 2.71- 2.54 (m, 2H), 2.48-2.28 (m, 2H), 1.54 (d, J = 5.4 Hz, 4H), 1.41-1.21 (m, 10H) | 921.4 | D | |
| I-71 | 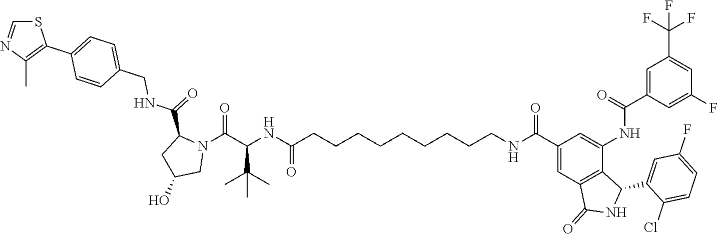 [SMILES: Cc1ncsc1-c1ccc(CNC(=O)[C@@H]2C[C@@H](O)CN2C(=O)[C@@H](NC(=O)CCCCCCCCCNC(=O)c2cc(NC(=O)c3cc(F)cc(C(F)(F)F)c3)c3c(c2)C(=O)N[C@H]3c2cc(F)ccc2Cl)C(C)(C)C)cc1] | 1H NMR (400 MHz, DMSO-d6) = 10.62 (s, 1H), 9.25 (s, 1H), 8.98 (s, 1H), 8.78 (t, J = 5.3 Hz, 1H), 8.56 (t, J = 6.0 Hz, 1H), 8.22 (s, 1H), 7.99 (s, 1H), 7.95 (d, J = 8.7 Hz, 1H), 7.84 (d, J = 9.4 Hz, 1H), 7.75 (d, J = 8.9 Hz, 1H), 7.68 (s, 1H), 7.44-7.35 (m, 5H), 7.32 (dd, J = 5.2, 8.9 Hz, 1H), 7.13-7.05 (m, 1H), 6.11-5.93 (m, 1H), 5.12 (d, J = 3.4 Hz, 1H), 4.54 (d, J = 9.4 Hz, 1H), 4.46-4.38 (m, 2H), 4.35 (s, 1H), 4.26-4.17 (m, 1H), 3.69- 3.59 (m, 2H), 3.29-3.23 (m, 2H), 2.44 (s, 3H), 2.31-2.21 (m, 1H), | 1093.85 | C | |
| | | 2.15-2.00 (m, 2H), 1.93-1.85 (m, | | | |
| | | 1H), 1.58-1.47 (m, 4H), 1.34-1.22 | | | |
| | | (m, 10H), 0.93 (s, 9H) | | | |
| I-72 | 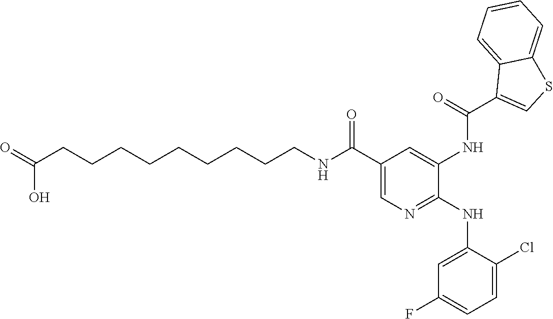 [SMILES: O=C(O)CCCCCCCCCNC(=O)c1cnc(Nc2cc(F)ccc2Cl)c(NC(=O)c2csc3ccccc23)c1] | 1H NMR (400 MHz, DMSO-d6) = 11.95 (s, 1H), 10.51 (s, 1H), 8.74 (s, 1H), 8.69 (d, J = 2.2 Hz, 1H), 8.52-8.46 (m, 2H), 8.31 (s, 1H), 8.22-8.09 (m, 3H), 7.53-7.44 (m, 3H), 6.90 (ddd, J = 3.1, 7.9, 8.8 Hz, 1H), 3.28-3.22 (m, 2H), 2.18 (t, J = 7.3 Hz, 2H), 1.57-1.43 (m, 4H), 1.33-1.23 (m, 10H) | 611.15 | A | D |
| I-73 | 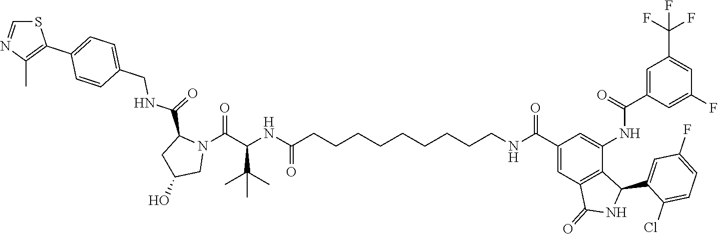 [SMILES: Cc1ncsc1-c1ccc(CNC(=O)[C@@H]2C[C@@H](O)CN2C(=O)[C@@H](NC(=O)CCCCCCCCCNC(=O)c2cc(NC(=O)c3cc(F)cc(C(F)(F)F)c3)c3c(c2)C(=O)N[C@@H]3c2cc(F)ccc2Cl)C(C)(C)C)cc1] | 1H NMR (400 MHz, DMSO-d6) = 10.61 (s, 1H), 9.25 (s, 1H), 8.98 (s, 1H), 8.78 (t, J = 5.4 Hz, 1H), 8.55 (t, J = 5.9 Hz, 1H), 8.22 (s, 1H), 8.00-7.94 (m, 2H), 7.84 (d, J = 9.4 Hz, 1H), 7.75 (d, J = 8.8 Hz, 1H), 7.68 (s, 1H), 7.44-7.35 (m, 5H), 7.33-7.29 (m, 1H), 7.09 (dt, J = 3.1, 8.4 Hz, 1H), 6.09-5.96 (m, 1H), 5.11 (d, J = 3.7 Hz, 1H), 4.54 (d, J = 9.5 Hz, 1H), 4.47-4.39 (m, 2H), 4.35 (s, 1H), 4.21 (dd, J = 5.5, 15.9 Hz, 1H), 3.69-3.62 (m, 2H), 3.29-3.25 (m, 2H), 2.46-2.44 (m, 3H), 2.26 (td, J = 7.4, 14.4 Hz, 1H), | 1093.85 | A | |
| | | 2.15-1.97 (m, 2H), 1.90 (ddd, J = | | | |
| | | 4.5, 8.5, 12.8 Hz, 1H), 1.57-1.47 | | | |
| | | (m, 4H), 1.30-1.24 (m, 10H), 0.93 | | | |
| | | (s, 9H) | | | |
| I-74 | 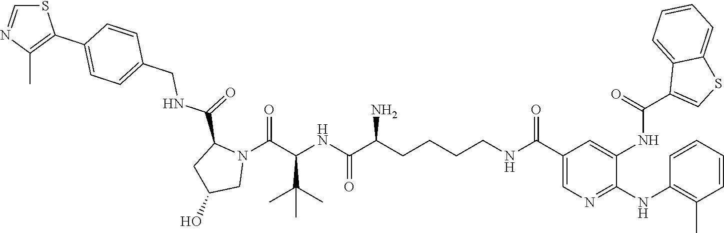 [SMILES: Cc1ccccc1Nc1ncc(C(=O)NCCCC[C@H](N)C(=O)N[C@H](C(=O)N2C[C@H](O)C[C@H]2C(=O)NCc2ccc(-c3scnc3C)cc2)C(C)(C)C)cc1NC(=O)c1csc2ccccc12] | 1H NMR (400 MHz, DMSO-d6) = 10.06 (s, 1H), 8.97 (s, 1H), 8.70 (s, 1H), 8.58 (t, J = 6.0 Hz, 1H), 8.50- 8.43 (m, 2H), 8.35-8.29 (m, 1H), 8.23-8.18 (m, 2H), 8.12-8.04 (m, 2H), 7.52-7.35 (m, 7H), 7.24-7.14 (m, 2H), 7.08-7.02 (m, 1H), 5.22- 5.06 (m, 1H), 4.50 (d, J = 9.5 Hz, 1H), 4.47-4.31 (m, 3H), 4.28-4.19 (m, 1H), 3.71-3.64 (m, 2H), 3.59 (d, J = 10.6 Hz, 2H), 3.22-3.17 (m, 2H), 2.44 (s, 3H), 2.17 (s, 3H), 2.08-2.00 (m, 1H), 1.95-1.88 (m, 1H), 1.71-1.61 (m, 1H), 1.55-1.31 | 944.35 | D | |
| | | (m, 5H), 0.94 (s, 9H) | | | |
| I-75 | 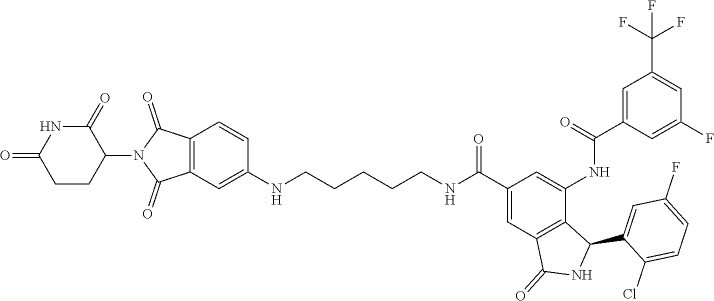 [SMILES: O=C1CCC(N2C(=O)c3ccc(NCCCCCNC(=O)c4cc(NC(=O)c5cc(F)cc(C(F)(F)F)c5)c5c(c4)C(=O)N[C@@H]5c4cc(F)ccc4Cl)cc3C2=O)C(=O)N1] | 1H NMR (400 MHz, DMSO-d6) = 11.05 (s, 1H), 10.61 (s, 1H), 9.28- 9.21 (m, 1H), 8.81 (t, J = 5.4 Hz, 1H), 8.23 (s, 1H), 8.00-7.91 (m, 2H), 7.75 (d, J = 9.1 Hz, 1H), 7.68 (s, 1H), 7.55 (d, J = 8.4 Hz, 1H), 7.32 (dd, J = 5.1, 8.9 Hz, 1H), 7.14-7.04 (m, 2H), 6.95 (d, J = 1.9 Hz, 1H), 6.85 (dd, J = 2.1, 8.4 Hz, 1H), 6.09-5.96 (m, 1H), 5.05-4.99 (m, 1H), 3.22-3.12 (m, 2H), 2.94- 2.81 (m, 1H), 2.62-2.54 (m, 2H), 2.02-1.93 (m, 2H), 1.62 (sxt, J = 7.2 Hz, 4H), 1.49-1.40 (m, 2H) | 852.2 | C | |
| I-76 | 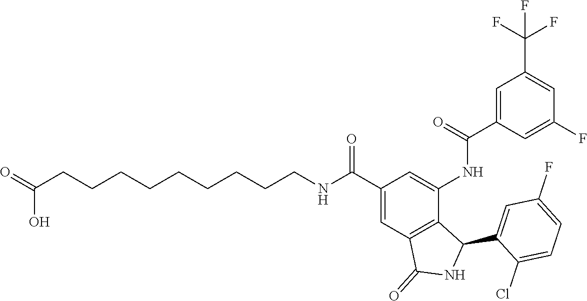 [SMILES: O=C(O)CCCCCCCCCNC(=O)c1cc(NC(=O)c2cc(F)cc(C(F)(F)F)c2)c2c(c1)C(=O)N[C@@H]2c1cc(F)ccc1Cl] | 1H NMR (400 MHz, DMSO-d6) = 11.99-11.83 (m, 1H), 10.62 (br s, 1H), 9.25 (br s, 1H), 8.79-8.74 (m, 1H), 8.22 (d, J = 0.9 Hz, 1H), 8.03- 7.91 (m, 2H), 7.79-7.64 (m, 2H), 7.32 (dd, J = 5.2, 8.9 Hz, 1H), 7.09 (dt, J = 3.0, 8.4 Hz, 1H), 6.09-5.87 (m, 1H), 3.30-3.25 (m, 2H), 2.19- 2.12 (m, 2H), 1.56-1.43 (m, 4H), 1.36-1.24 (m, 10H) | 680.25 | A | |
| I-77 | 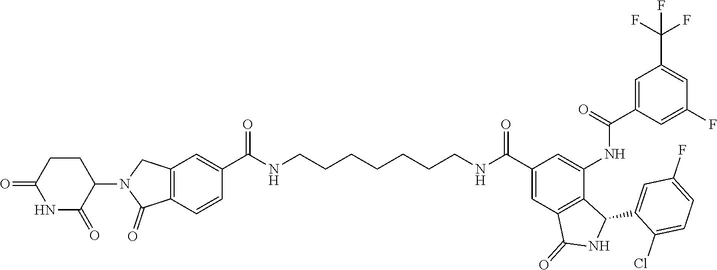 [SMILES: O=C1CCC(N2Cc3cc(C(=O)NCCCCCCCNC(=O)c4cc(NC(=O)c5cc(F)cc(C(F)(F)F)c5)c5c(c4)C(=O)N[C@H]5c4cc(F)ccc4Cl)ccc3C2=O)C(=O)N1] | 1H NMR (400 MHz, DMSO-d6) = 11.00 (s, 1H), 10.61 (s, 1H), 9.25 (br s, 1H), 8.79 (t, J = 5.6 Hz, 1H), 8.63 (t, J = 5.6 Hz, 1H), 8.22 (s, 1H), 8.04 (s, 1H), 8.00-7.93 (m, 3H), 7.81-7.73 (m, 2H), 7.68 (s, 1H), 7.32 (dd, J = 5.2, 8.9 Hz, 1H), 7.09 (dt, J = 2.9, 8.4 Hz, 1H), 6.11- 5.94 (m, 1H), 5.14 (dd, J = 5.1, 13.3 Hz, 1H), 4.55-4.48 (m, 1H), 4.43-4.33 (m, 1H), 3.31-3.21 (m, 4H), 2.97-2.84 (m, 1H), 2.63-2.54 (m, 2H), 2.45-2.37 (m, 1H), 2.05- 1.97 (m, 1H), 1.55 (br s, 4H), 1.35 (br s, 6H) | 893.15 | A | |
| I-78 | 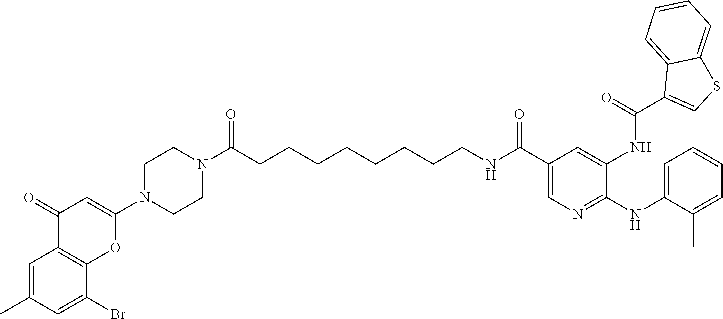 [SMILES: Cc1cc(Br)c2oc(N3CCN(C(=O)CCCCCCCCNC(=O)c4cnc(Nc5ccccc5C)c(NC(=O)c5csc6ccccc56)c4)CC3)cc(=O)c2c1] | 1H NMR (400 MHz, DMSO-d6) = 10.05 (s, 1H), 8.70 (s, 1H), 8.51- 8.48 (m, 1H), 8.45 (d, J = 2.2 Hz, 1H), 8.32 (t, J = 5.6 Hz, 1H), 8.18 (s, 1H), 8.12-8.07 (m, 2H), 7.79 (dd, J = 0.6, 2.2 Hz, 1H), 7.70 (dd, J = 0.8, 2.1 Hz, 1H), 7.51-7.43 (m, 3H), 7.23-7.15 (m, 2H), 7.08-7.01 (m, 1H), 5.54 (s, 1H), 3.64-3.58 (m, 6H), 3.55 (d, J = 5.4 Hz, 2H), 3.26-3.21 (m, 2H), 2.38 (s, 3H), 2.36-2.33 (m, 2H), 2.17 (s, 3H), 1.51 (d, J = 4.4 Hz, 4H), 1.29 (br s, 8H) | 865.7 | B | D |
| I-79 | 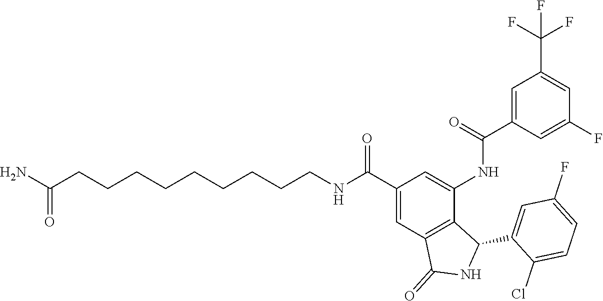 [SMILES: NC(=O)CCCCCCCCCNC(=O)c1cc(NC(=O)c2cc(F)cc(C(F)(F)F)c2)c2c(c1)C(=O)N[C@H]2c1cc(F)ccc1Cl] | 1H NMR (400 MHz, DMSO-d6) = 10.62 (br s, 1H), 9.25 (br s, 1H), 8.78 (t, J = 5.5 Hz, 1H), 8.22 (d, J = 0.7 Hz, 1H), 8.00-7.91 (m, 2H), 7.75 (d, J = 8.9 Hz, 1H), 7.68 (s, 1H), 7.32 (dd, J = 5.1, 8.9 Hz, 1H), 7.20 (br s, 1H), 7.11-7.04 (m, 1H), 6.66 (br s, 1H), 6.10-5.94 (m, 1H), 3.29 (d, J = 6.6 Hz, 2H), 2.03-1.92 (m, 2H), 1.58-1.50 (m, 2H), 1.50- 1.42 (m, 2H), 1.33-1.21 (m, 10H) | 679.3 | D | |
| I-80 | 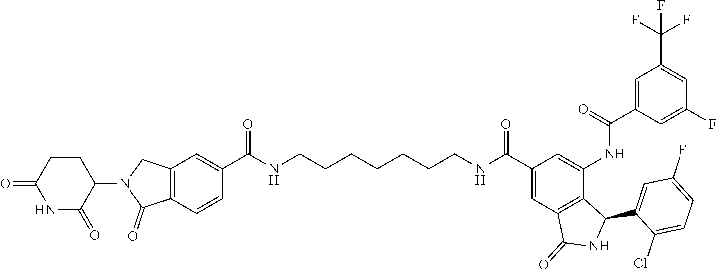 [SMILES: O=C1CCC(N2Cc3cc(C(=O)NCCCCCCCNC(=O)c4cc(NC(=O)c5cc(F)cc(C(F)(F)F)c5)c5c(c4)C(=O)N[C@@H]5c4cc(F)ccc4Cl)ccc3C2=O)C(=O)N1] | 1H NMR (400 MHz, DMSO-d6) = 11.00 (s, 1H), 10.61 (s, 1H), 9.29- 9.20 (m, 1H), 8.79 (t, J = 5.4 Hz, 1H), 8.67-8.62 (m, 1H), 8.22 (s, 1H), 8.04 (s, 1H), 8.00-7.93 (m, 3H), 7.81-7.71 (m, 2H), 7.69-7.65 (m, 1H), 7.34-7.28 (m, 1H), 7.13- 7.05 (m, 1H), 6.09-5.95 (m, 1H), 5.14 (dd, J = 5.2, 13.3 Hz, 1H), 4.55-4.46 (m, 1H), 4.43-4.33 (m, 1H), 3.30-3.23 (m, 4H), 2.99-2.87 (m, 1H), 2.65-2.54 (m, 2H), 2.45- 2.37 (m, 1H), 2.07-1.95 (m, 1H), 1.55 (br s, 4H), 1.35 (br s, 6H) | 893.15 | B | |
| I-81 | 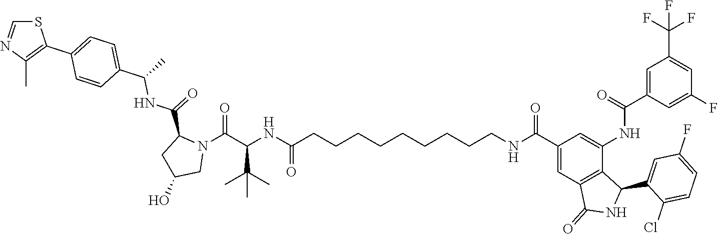 [SMILES: Cc1ncsc1-c1ccc([C@H](C)NC(=O)[C@@H]2C[C@@H](O)CN2C(=O)[C@@H](NC(=O)CCCCCCCCCNC(=O)c2cc(NC(=O)c3cc(F)cc(C(F)(F)F)c3)c3c(c2)C(=O)N[C@@H]3c2cc(F)ccc2Cl)C(C)(C)C)cc1] | 1H NMR (400 MHz, DMSO-d6) = 10.62 (br s, 1H), 9.25 (br s, 1H), 8.98 (s, 1H), 8.78 (t, J = 5.6 Hz, 1H), 8.37 (d, J = 7.9 Hz, 1H), 8.22 (s, 1H), 8.02-7.93 (m, 2H), 7.80- 7.72 (m, 2H), 7.68 (s, 1H), 7.45- 7.29 (m, 6H), 7.12-7.05 (m, 1H), 6.09-5.97 (m, 1H), 5.09 (d, J = 3.5 Hz, 1H), 4.96 (s, 1H), 4.54-4.47 (m, 1H), 4.43-4.37 (m, 1H), 4.29- 4.23 (m, 1H), 3.63-3.57 (m, 2H), 2.45 (s, 3H), 2.27-2.19 (m, 1H), 2.14-2.07 (m, 1H), 2.04-1.94 (m, 2H), 1.79 (ddd, J = 4.7, 8.4, 12.7 Hz, 1H), 1.57-1.47 (m, 4H), 1.37 | 1106.35 | B | |
| | | (d, J = 6.9 Hz, 3H), 1.28-1.23 (m, | | | |
| | | 11H), 0.92 (s, 9H) | | | |
| I-82 | 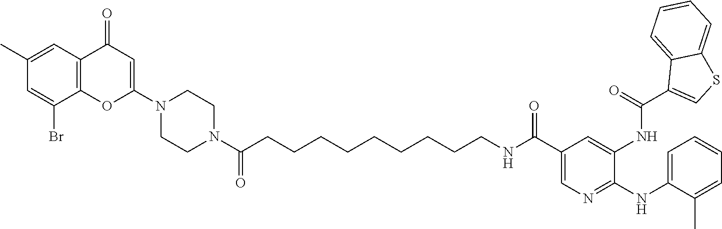 [SMILES: Cc1cc(Br)c2oc(N3CCN(C(=O)CCCCCCCCCNC(=O)c4cnc(Nc5ccccc5C)c(NC(=O)c5csc6ccccc56)c4)CC3)cc(=O)c2c1] | 1H NMR (400 MHz, DMSO-d6) = 10.05 (s, 1H), 8.70 (s, 1H), 8.49 (dd, J = 1.3, 7.2 Hz, 1H), 8.44 (d, J = 2.2 Hz, 1H), 8.33-8.29 (m, 1H), 8.19-8.17 (m, 1H), 8.12-8.07 (m, 2H), 7.80 (d, J = 2.2 Hz, 1H), 7.70 (dd, J = 0.8, 2.0 Hz, 1H), 7.50- 7.42 (m, 3H), 7.24-7.15 (m, 3H), 5.54 (s, 1H), 3.62 (s, 6H), 3.58- 3.53 (m, 2H), 3.26-3.21 (m, 2H), 2.38 (s, 3H), 2.17 (s, 3H), 2.02- 1.95 (m, 2H), 1.52-1.48 (m, 4H), 1.24 (br s, 10H) | 879.45 | B | D |
| I-83 | 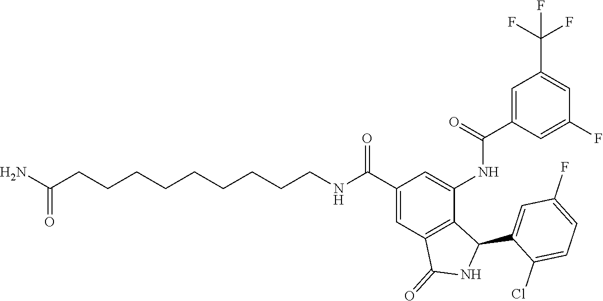 [SMILES: NC(=O)CCCCCCCCCNC(=O)c1cc(NC(=O)c2cc(F)cc(C(F)(F)F)c2)c2c(c1)C(=O)N[C@@H]2c1cc(F)ccc1Cl] | 1H NMR (400 MHz, DMSO-d6) = 10.74-10.61 (m, 1H), 9.25 (br s, 1H), 8.79 (t, J = 5.6 Hz, 1H), 8.21 (s, 1H), 8.03-7.87 (m, 2H), 7.83- 7.62 (m, 2H), 7.33-7.28 (m, 2H), 7.24-7.18 (m, 1H), 7.09 (dt, J = 3.0, 8.4 Hz, 1H), 6.66 (br s, 1H), 6.10-5.93 (m, 1H), 3.21 (br s, 2H), 2.03-1.95 (m, 2H), 1.60-1.45 (m, 4H), 1.31-1.21 (m, 10H) | 679.25 | A | |
| I-84 | 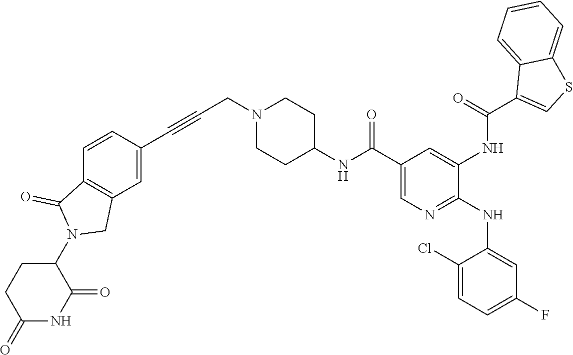 [SMILES: O=C1CCC(N2Cc3cc(C#CCN4CCC(NC(=O)c5cnc(Nc6cc(F)ccc6Cl)c(NC(=O)c6csc7ccccc67)c5)CC4)ccc3C2=O)C(=O)N1] | 1H NMR (400 MHz, DMSO-d6) = 11.00 (s, 1H), 10.52 (s, 1H), 8.75 (s, 1H), 8.72 (d, J = 2.1 Hz, 1H), 8.52-8.48 (m, 1H), 8.37-8.31 (m, 2H), 8.23-8.08 (m, 3H), 7.75-7.67 (m, 2H), 7.62-7.45 (m, 4H), 6.95- 6.85 (m, 1H), 5.13-5.06 (m, 1H), 4.50-4.42 (m, 1H), 4.38-4.29 (m, 1H), 3.60-3.53 (m, 1H), 2.94-2.87 (m, 2H), 2.64-2.57 (m, 2H), 2.41- 2.36 (m, 1H), 2.04-1.96 (m, 2H), 1.93-1.80 (m, 2H), 1.70-1.56 (m, 2H), 1.51-1.24 (m, 3H) | 804.6 | D | |
| I-85 | 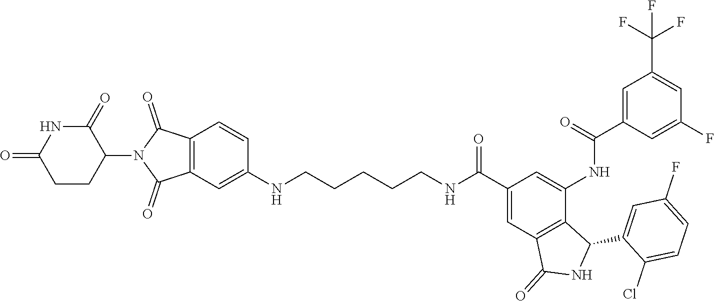 [SMILES: O=C1CCC(N2C(=O)c3ccc(NCCCCCNC(=O)c4cc(NC(=O)c5cc(F)cc(C(F)(F)F)c5)c5c(c4)C(=O)N[C@H]5c4cc(F)ccc4Cl)cc3C2=O)C(=O)N1] | 1H NMR (400 MHz, DMSO-d6) = 11.05 (s, 1H), 10.61 (s, 1H), 9.25 (br s, 1H), 8.81 (t, J = 5.5 Hz, 1H), 8.23 (s, 1H), 8.00-7.93 (m, 2H), 7.75 (d, J = 8.9 Hz, 1H), 7.68 (s, 1H), 7.55 (d, J = 8.4 Hz, 1H), 7.34- 7.30 (m, 1H), 7.13-7.05 (m, 2H), 6.95 (d, J = 1.9 Hz, 1H), 6.85 (dd, J = 2.1, 8.4 Hz, 1H), 6.08-5.95 (m, 1H), 5.02 (dd, J = 5.4, 12.8 Hz, 1H), 3.23-3.13 (m, 2H), 2.91-2.80 (m, 1H), 2.61-2.53 (m, 2H), 2.05- 1.94 (m, 2H), 1.62 (sxt, J = 7.4 Hz, 4H), 1.50-1.42 (m, 2H) | 851.65 | A | |
| I-86 | 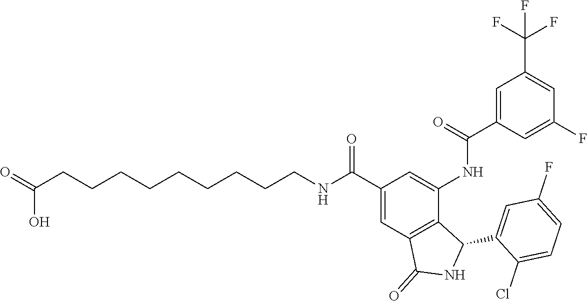 [SMILES: O=C(O)CCCCCCCCCNC(=O)c1cc(NC(=O)c2cc(F)cc(C(F)(F)F)c2)c2c(c1)C(=O)N[C@H]2c1cc(F)ccc1Cl] | 1H NMR (400 MHz, DMSO-d6) = 12.06-11.86 (m, 1H), 10.63 (br s, 1H), 9.25 (br s, 1H), 8.78 (t, J = 5.5 Hz, 1H), 8.22 (s, 1H), 8.02-7.92 (m, 2H), 7.79-7.62 (m, 2H), 7.32 (dd, J = 5.2, 8.9 Hz, 1H), 7.09 (dt, J = 3.0, 8.3 Hz, 1H), 6.09-5.89 (m, 1H), 3.30-3.26 (m, 2H), 2.29-2.07 (m, 2H), 1.60-1.43 (m, 4H), 1.32- 1.21 (m, 10H) | 680.25 | D | |
| I-87 | 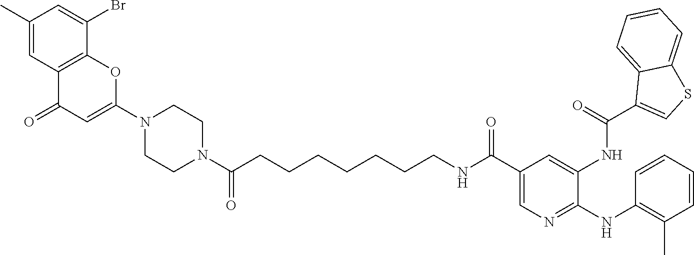 [SMILES: Cc1cc(Br)c2oc(N3CCN(C(=O)CCCCCCCNC(=O)c4cnc(Nc5ccccc5C)c(NC(=O)c5csc6ccccc56)c4)CC3)cc(=O)c2c1] | 1H NMR (400 MHz, DMSO-d6) = 10.11 (s, 1H), 8.71 (s, 1H), 8.52 (s, 1H), 8.44 (s, 1H), 8.33-8.28 (m, 1H), 8.21 (br s, 1H), 8.12 (br s, 1H), 8.08 (d, J = 7.2 Hz, 1H), 7.79 (d, J = 1.5 Hz, 1H), 7.70 (dd, J = 0.7, 2.1 Hz, 1H), 7.51-7.42 (m, 3H), 7.23-7.15 (m, 2H), 7.06-6.99 (m, 1H), 5.54 (s, 1H), 3.64-3.58 (m, 6H), 3.56 (d, J = 4.7 Hz, 2H), 3.24 (d, J = 6.3 Hz, 2H), 2.38 (s, 3H), 2.17 (s, 3H), 2.05-1.97 (m, 1H), 1.51 (br s, 4H), 1.32-1.23 (m, 8H) | 849.2 | B | A |
| I-88 | 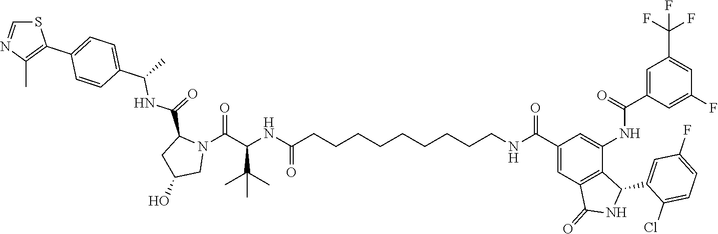 [SMILES: Cc1ncsc1-c1ccc([C@H](C)NC(=O)[C@@H]2C[C@@H](O)CN2C(=O)[C@@H](NC(=O)CCCCCCCCCNC(=O)c2cc(NC(=O)c3cc(F)cc(C(F)(F)F)c3)c3c(c2)C(=O)N[C@H]3c2cc(F)ccc2Cl)C(C)(C)C)cc1] | 1H NMR (400 MHz, DMSO-d6) = 10.61 (s, 1H), 9.32-9.18 (m, 1H), 8.98 (s, 1H), 8.78 (t, J = 5.4 Hz, 1H), 8.36 (d, J = 7.8 Hz, 1H), 8.27- 8.20 (m, 1H), 8.04-7.92 (m, 2H), 7.79-7.67 (m, 3H), 7.47-7.24 (m, 6H), 7.17-7.03 (m, 1H), 6.13-5.94 (m, 1H), 5.09 (d, J = 3.5 Hz, 1H), 4.99-4.80 (m, 1H), 4.53-4.47 (m, 1H), 4.42 (t, J = 8.0 Hz, 1H), 4.27 (d, J = 3.2 Hz, 1H), 3.63-3.55 (m, 2H), 2.45 (s, 3H), 2.29-2.19 (m, 1H), 2.15-2.06 (m, 1H), 2.03-1.96 (m, 2H), 1.82-1.75 (m, 1H), 1.56- 1.45 (m, 4H), 1.37 (d, J = 6.9 Hz, | 1106.45 | C | |
| | | 3H), 1.27-1.22 (m, 11H), 0.93 (s, | | | |
| | | 9H) | | | |
| I-89 | 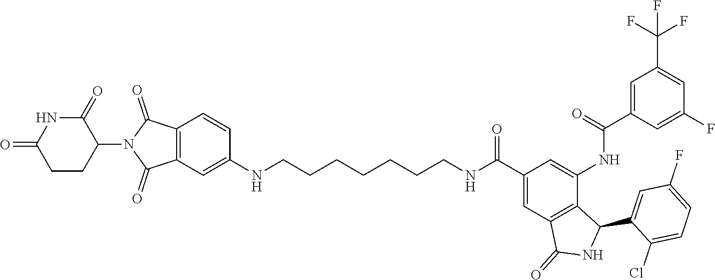 [SMILES: O=C1CCC(N2C(=O)c3ccc(NCCCCCCCNC(=O)c4cc(NC(=O)c5cc(F)cc(C(F)(F)F)c5)c5c(c4)C(=O)N[C@@H]5c4cc(F)ccc4Cl)cc3C2=O)C(=O)N1] | 1H NMR (400 MHz, DMSO-d6) = 11.05 (br s, 1H), 10.74-10.63 (m, 1H), 9.23 (br s, 1H), 8.78 (d, J = 5.6 Hz, 1H), 8.51 (s, 1H), 8.20 (br s, 1H), 8.04-7.88 (m, 2H), 7.76 (d, J = 8.8 Hz, 1H), 7.70 (s, 1H), 7.55 (d, J = 8.4 Hz, 1H), 7.32 (dd, J = 5.1, 8.9 Hz, 1H), 7.14-7.02 (m, 2H), 6.94 (d, J = 1.8 Hz, 1H), 6.84 (dd, J = 2.0, 8.4 Hz, 1H), 6.10-5.92 (m, 1H), 5.02 (dd, J = 5.4, 12.8 Hz, 1H), 3.26 (br s, 2H), 3.19-3.10 (m, 2H), 2.92-2.82 (m, 1H), 2.60-2.53 (m, 2H), 2.02-1.94 (m, 1H), 1.62- 1.50 (m, 4H), 1.37 (br s, 6H) | 879.2 | C | |
| I-90 | 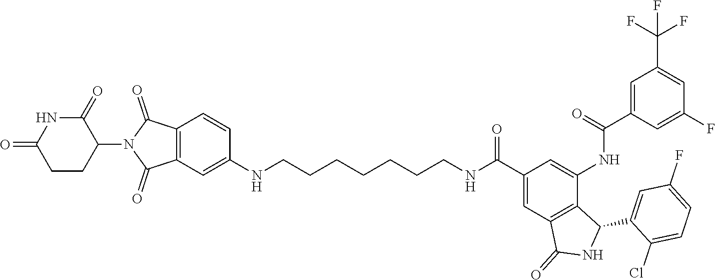 [SMILES: O=C1CCC(N2C(=O)c3ccc(NCCCCCCCNC(=O)c4cc(NC(=O)c5cc(F)cc(C(F)(F)F)c5)c5c(c4)C(=O)N[C@H]5c4cc(F)ccc4Cl)cc3C2=O)C(=O)N1] | 1H NMR (400 MHz, DMSO-d6) = 11.05 (s, 1H), 10.62 (s, 1H), 9.25 (br s, 1H), 8.81-8.77 (m, 1H), 8.22 (s, 1H), 8.01-7.91 (m, 2H), 7.75 (d, J = 9.1 Hz, 1H), 7.68 (s, 1H), 7.55 (d, J = 8.4 Hz, 1H), 7.32 (dd, J = 5.2, 8.9 Hz, 1H), 7.14-7.05 (m, 2H), 6.94 (d, J = 1.9 Hz, 1H), 6.84 (dd, J = 2.1, 8.5 Hz, 1H), 6.10-5.96 (m, 1H), 5.02 (dd, J = 5.5, 12.8 Hz, 1H), 3.30-3.27 (m, 2H), 3.18- 3.10 (m, 2H), 2.93-2.81 (m, 1H), 2.61-2.54 (m, 2H), 2.02-1.94 (m, 1H), 1.61-1.52 (m, 4H), 1.37 (br s, 6H) | 879.4 | A | |
| I-91 | 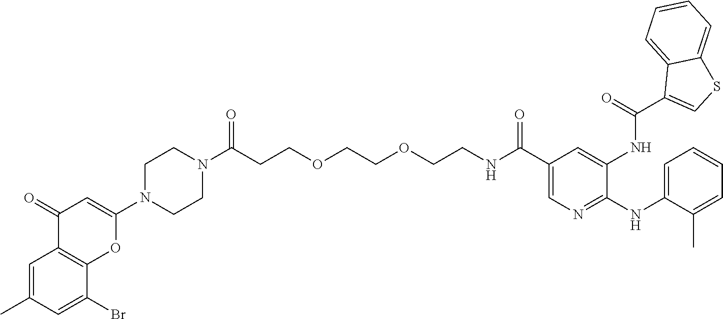 [SMILES: Cc1cc(Br)c2oc(N3CCN(C(=O)CCOCCOCCNC(=O)c4cnc(Nc5ccccc5C)c(NC(=O)c5csc6ccccc56)c4)CC3)cc(=O)c2c1] | 1H NMR (400 MHz, DMSO-d6) = 10.04 (s, 1H), 8.70 (s, 1H), 8.50- 8.47 (m, 1H), 8.45 (d, J = 2.2 Hz, 1H), 8.43-8.38 (m, 1H), 8.20 (s, 1H), 8.13 (d, J = 2.1 Hz, 1H), 8.10- 8.06 (m, 1H), 7.78 (dd, J = 0.6, 2.1 Hz, 1H), 7.69 (dd, J = 0.7, 2.1 Hz, 1H), 7.50-7.41 (m, 3H), 7.23-7.14 (m, 2H), 7.08-7.01 (m, 1H), 5.53 (s, 1H), 3.67-3.57 (m, 8H), 3.56- 3.50 (m, 8H), 3.43-3.39 (m, 2H), 2.61 (t, J = 6.5 Hz, 2H), 2.37 (s, 3H), 2.17 (s, 3H) | 868.8 | D | |
| I-92 | 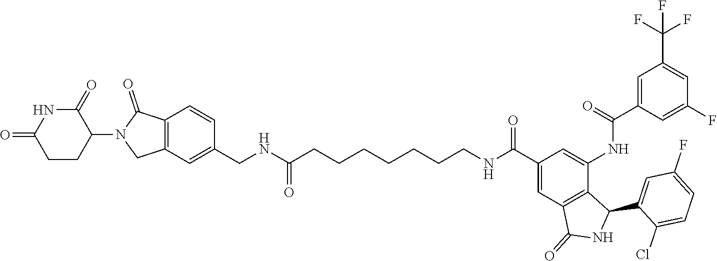 [SMILES: O=C(CCCCCCCNC(=O)c1cc(NC(=O)c2cc(F)cc(C(F)(F)F)c2)c2c(c1)C(=O)N[C@@H]2c1cc(F)ccc1Cl)NCc1ccc2c(c1)CN(C1CCC(=O)NC1=O)C2=O] | 1H NMR (400 MHz, DMSO-d6) = 10.97 (br s, 1H), 10.61 (s, 1H), 9.25 (br s, 1H), 8.78 (t, J = 5.5 Hz, 1H), 8.40 (t, J = 5.9 Hz, 1H), 8.22 (s, 1H), 7.99-7.92 (m, 2H), 7.75 (d, J = 8.9 Hz, 1H), 7.69-7.65 (m, 2H), 7.45 (s, 1H), 7.41-7.28 (m, 2H), 7.09 (dt, J = 2.9, 8.4 Hz, 1H), 6.09- 5.96 (m, 1H), 5.10 (dd, J = 5.1, 13.3 Hz, 1H), 4.47-4.26 (m, 4H), 3.30- 3.22 (m, 2H), 2.97-2.85 (m, 1H), 2.67-2.54 (m, 2H), 2.41-2.31 (m, 1H), 2.16 (t, J = 7.5 Hz, 2H), 2.04- 1.96 (m, 1H), 1.59-1.48 (m, 4H), 1.36-1.25 (m, 6H) | 907.2 | C | |
| I-93 | 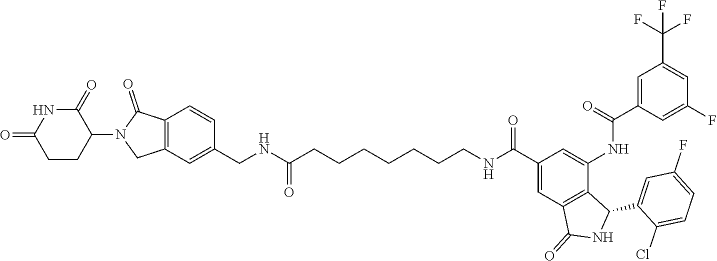 [SMILES: O=C(CCCCCCCNC(=O)c1cc(NC(=O)c2cc(F)cc(C(F)(F)F)c2)c2c(c1)C(=O)N[C@H]2c1cc(F)ccc1Cl)NCc1ccc2c(c1)CN(C1CCC(=O)NC1=O)C2=O] | 1H NMR (400 MHz, DMSO-d6) = 10.97 (s, 1H), 10.61 (s, 1H), 9.30- 9.21 (m, 1H), 8.78 (t, J = 5.5 Hz, 1H), 8.43-8.36 (m, 1H), 8.22 (s, 1H), 8.00-7.93 (m, 2H), 7.75 (d, J = 8.8 Hz, 1H), 7.68-7.64 (m, 2H), 7.45 (s, 1H), 7.40-7.29 (m, 2H), 7.09 (dt, J = 3.1, 8.4 Hz, 1H), 6.11- 5.94 (m, 1H), 5.10 (dd, J = 5.1, 13.2 Hz, 1H), 4.47-4.23 (m, 4H), 3.31-3.24 (m, 2H), 2.97-2.85 (m, 1H), 2.68-2.53 (m, 2H), 2.42-2.31 (m, 1H), 2.19-2.12 (m, 2H), 2.05- 1.94 (m, 1H), 1.60-1.47 (m, 4H), 1.38-1.25 (m, 6H) | 907.2 | A | |
| I-94 | 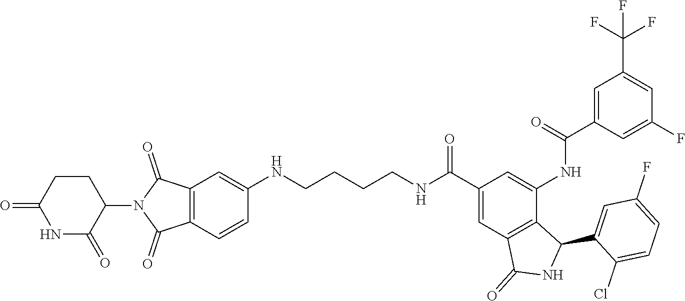 [SMILES: O=C1CCC(N2C(=O)c3ccc(NCCCCNC(=O)c4cc(NC(=O)c5cc(F)cc(C(F)(F)F)c5)c5c(c4)C(=O)N[C@@H]5c4cc(F)ccc4Cl)cc3C2=O)C(=O)N1] | 1H NMR (400 MHz, DMSO-d6) = 11.05 (s, 1H), 10.61 (s, 1H), 9.34- 9.20 (m, 1H), 8.84 (t, J = 5.4 Hz, 1H), 8.23 (s, 1H), 8.00 (s, 1H), 7.95 (d, J = 8.4 Hz, 1H), 7.75 (d, J = 8.9 Hz, 1H), 7.68 (s, 1H), 7.56 (d, J = 8.4 Hz, 1H), 7.32 (dd, J = 5.2, 8.9 Hz, 1H), 7.17-7.06 (m, 2H), 6.96 (d, J = 1.9 Hz, 1H), 6.86 (dd, J = 2.1, 8.4 Hz, 1H), 6.10-5.95 (m, 1H), 5.02 (dd, J = 5.4, 12.9 Hz, 1H), 3.45-3.38 (m, 2H), 3.22 (br s, 2H), 2.94-2.81 (m, 1H), 2.60-2.54 (m, 2H), 2.02-1.95 (m, 2H), 1.71- 1.59 (m, 4H) | 837.65 | C | |
| I-95 | 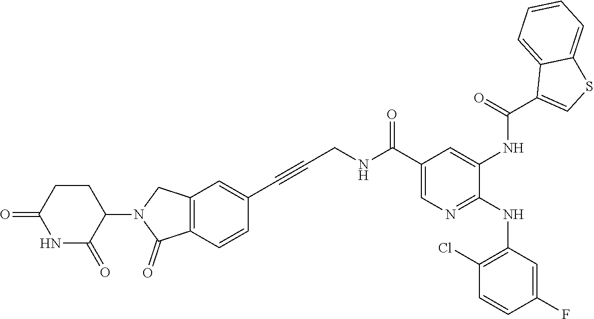 [SMILES: O=C1CCC(N2Cc3cc(C#CCNC(=O)c4cnc(Nc5cc(F)ccc5Cl)c(NC(=O)c5csc6ccccc56)c4)ccc3C2=O)C(=O)N1] | 1H NMR (400 MHz, DMSO-d6) = 10.99 (br s, 1H), 10.70-10.46 (m, 1H), 9.09 (br s, 1H), 8.73-8.58 (m, 2H), 8.49 (br s, 3H), 8.40-8.22 (m, 1H), 8.09 (d, J = 7.6 Hz, 1H), 7.72- 7.69 (m, 2H), 7.60-7.43 (m, 4H), 6.95-6.84 (m, 1H), 5.11 (dd, J = 5.1, 13.2 Hz, 1H), 4.51-4.30 (m, 4H), 2.95-2.86 (m, 1H), 2.44-2.36 (m, 1H), 2.03-1.95 (m, 2H) | 721.45 | A | |
| I-96 | 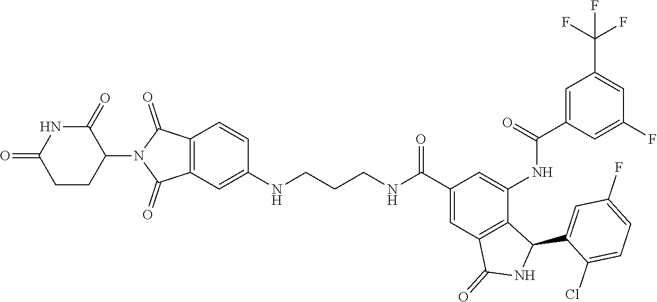 [SMILES: O=C1CCC(N2C(=O)c3ccc(NCCCNC(=O)c4cc(NC(=O)c5cc(F)cc(C(F)(F)F)c5)c5c(c4)C(=O)N[C@@H]5c4cc(F)ccc4Cl)cc3C2=O)C(=O)N1] | 1H NMR (400 MHz, DMSO-d6) = 11.06 (s, 1H), 10.62 (s, 1H), 9.26 (br s, 1H), 8.87 (t, J = 5.4 Hz, 1H), 8.24 (s, 1H), 8.00 (s, 1H), 7.95 (d, J = 8.4 Hz, 1H), 7.75 (d, J = 8.9 Hz, 1H), 7.68 (s, 1H), 7.58 (d, J = 8.4 Hz, 1H), 7.32 (dd, J = 5.2, 8.9 Hz, 1H), 7.18-7.04 (m, 2H), 6.98 (d, J = 1.8 Hz, 1H), 6.88 (dd, J = 2.0, 8.4 Hz, 1H), 6.09-5.93 (m, 1H), 5.07-4.97 (m, 1H), 3.47-3.42 (m, 2H), 3.27 (d, J = 6.0 Hz, 2H), 2.91-2.82 (m, 1H), 2.62-2.54 (m, 2H), 2.05-1.96 (m, 1H), 1.92-1.84 (m, 2H) | 822.75 | B | |
| I-97 | 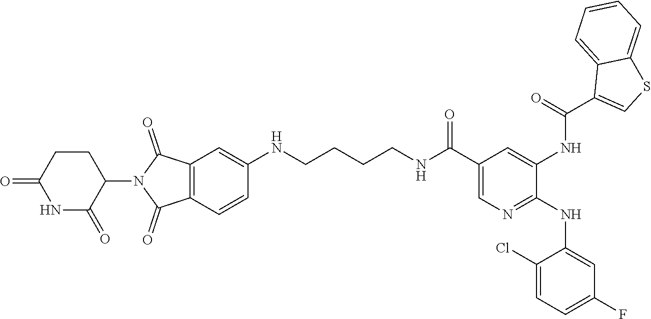 [SMILES: O=C1CCC(N2C(=O)c3ccc(NCCCCNC(=O)c4cnc(Nc5cc(F)ccc5Cl)c(NC(=O)c5csc6ccccc56)c4)cc3C2=O)C(=O)N1] | 1H NMR (400 MHz, DMSO-d6) = 11.05 (s, 1H), 10.50 (s, 1H), 8.74 (s, 1H), 8.71 (d, J = 2.2 Hz, 1H), 8.56 (t, J = 5.6 Hz, 1H), 8.51-8.47 (m, 1H), 8.32 (s, 1H), 8.22 (d, J = 2.2 Hz, 1H), 8.19-8.15 (m, 1H), 8.14-8.10 (m, 1H), 7.57-7.45 (m, 4H), 7.14 (t, J = 5.3 Hz, 1H), 6.96 (d, J = 1.9 Hz, 1H), 6.94-6.83 (m, 2H), 5.05-4.98 (m, 1H), 3.41-3.34 (m, 2H), 3.21 (d, J = 5.3 Hz, 2H), 2.92-2.81 (m, 1H), 2.60-2.53 (m, 2H), 2.03-1.93 (m, 1H), 1.69-1.60 (m, 4H) | 767.85 | B | |
| I-98 | 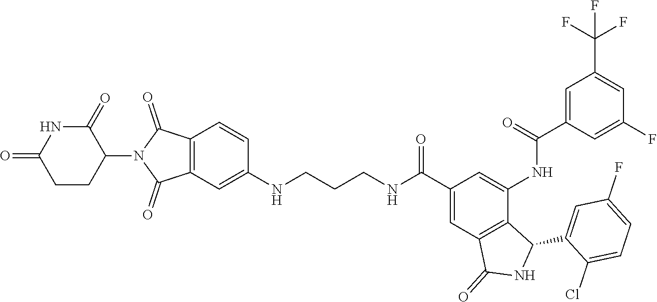 [SMILES: O=C1CCC(N2C(=O)c3ccc(NCCCNC(=O)c4cc(NC(=O)c5cc(F)cc(C(F)(F)F)c5)c5c(c4)C(=O)N[C@H]5c4cc(F)ccc4Cl)cc3C2=O)C(=O)N1] | 1H NMR (400 MHz, DMSO-d6) = 11.06 (s, 1H), 10.62 (s, 1H), 9.26 (br s, 1H), 8.87 (t, J = 5.4 Hz, 1H), 8.24 (s, 1H), 8.00 (s, 1H), 7.97-7.93 (m, 1H), 7.75 (d, J = 8.9 Hz, 1H), 7.68 (s, 1H), 7.58 (d, J = 8.4 Hz, 1H), 7.32 (dd, J = 5.2, 8.9 Hz, 1H), 7.19-7.14 (m, 1H), 7.12-7.05 (m, 1H), 7.00-6.96 (m, 1H), 6.88 (dd, J = 2.0, 8.4 Hz, 1H), 6.13-5.89 (m, 1H), 5.03 (dd, J = 5.4, 12.8 Hz, 1H), 3.48-3.39 (m, 2H), 3.30-3.23 (m, 2H), 2.92-2.80 (m, 1H), 2.63- 2.53 (m, 2H), 2.04-1.96 (m, 1H), 1.93-1.85 (m, 2H) | 822.75 | A | |
| I-99 | 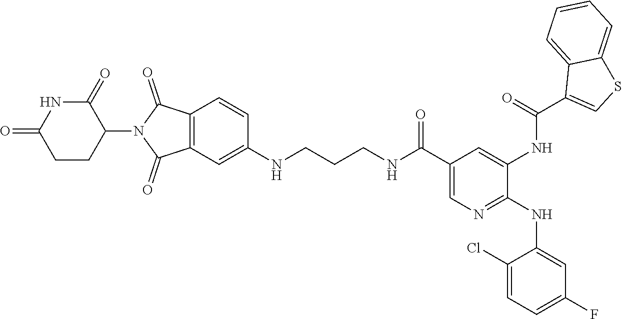 [SMILES: O=C1CCC(N2C(=O)c3ccc(NCCCNC(=O)c4cnc(Nc5cc(F)ccc5Cl)c(NC(=O)c5csc6ccccc56)c4)cc3C2=O)C(=O)N1] | 1H NMR (400 MHz, DMSO-d6) = 11.05 (s, 1H), 10.56-10.46 (m, 1H), 8.75-8.66 (m, 2H), 8.60-8.55 (m, 1H), 8.53-8.47 (m, 1H), 8.44- 8.32 (m, 1H), 8.27-8.13 (m, 2H), 8.11 (d, J = 7.0 Hz, 1H), 7.57 (d, J = 8.4 Hz, 1H), 7.54-7.44 (m, 3H), 7.16 (t, J = 5.3 Hz, 1H), 6.98 (d, J = 1.9 Hz, 1H), 6.93-6.85 (m, 2H), 5.02 (dd, J = 5.4, 12.8 Hz, 1H), 3.44-3.38 (m, 2H), 3.28-3.22 (m, 2H), 2.92-2.80 (m, 1H), 2.58 (d, J = 2.6 Hz, 1H), 2.03-1.94 (m, 2H), 1.90-1.81 (m, 2H) | 753.8 | B | |
| I-100 | 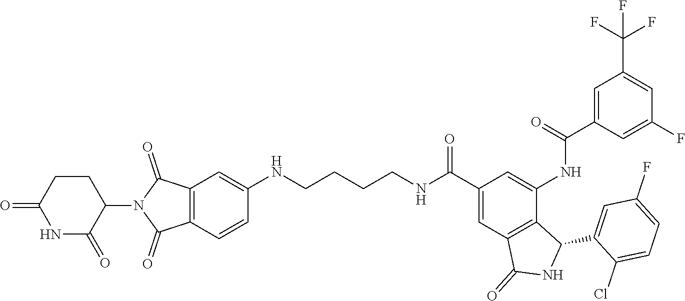 [SMILES: O=C1CCC(N2C(=O)c3ccc(NCCCCNC(=O)c4cc(NC(=O)c5cc(F)cc(C(F)(F)F)c5)c5c(c4)C(=O)N[C@H]5c4cc(F)ccc4Cl)cc3C2=O)C(=O)N1] | 1H NMR (400 MHz, DMSO-d6) = 11.05 (s, 1H), 10.61 (s, 1H), 9.25 (br s, 1H), 8.84 (t, J = 5.6 Hz, 1H), 8.23 (s, 1H), 8.00 (s, 1H), 7.95 (d, J = 8.4 Hz, 1H), 7.75 (d, J = 8.9 Hz, 1H), 7.68 (s, 1H), 7.56 (d, J = 8.4 Hz, 1H), 7.32 (dd, J = 5.1, 8.9 Hz, 1H), 7.17-7.04 (m, 2H), 6.96 (d, J = 1.9 Hz, 1H), 6.86 (dd, J = 2.0, 8.4 Hz, 1H), 6.10-5.91 (m, 1H), 5.02 (dd, J = 5.4, 12.8 Hz, 1H), 3.36 (dd, J = 2.2, 6.2 Hz, 2H), 3.22 (d, J = 5.1 Hz, 2H), 2.93-2.80 (m, 1H), 2.61-2.54 (m, 2H), 2.04-1.95 (m, 1H), 1.71- 1.61 (m, 4H) | 838.1 | A | |
| I-101 | 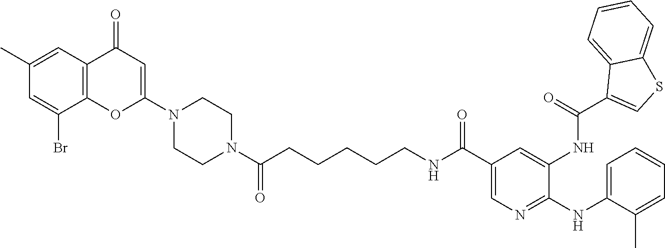 [SMILES: Cc1cc(Br)c2oc(N3CCN(C(=O)CCCCCNC(=O)c4cnc(Nc5ccccc5C)c(NC(=O)c5csc6ccccc56)c4)CC3)cc(=O)c2c1] | 1H NMR (400 MHz, DMSO-d6) = 10.07 (s, 1H), 8.70 (s, 1H), 8.52- 8.47 (m, 1H), 8.45 (d, J = 2.1 Hz, 1H), 8.33 (t, J = 5.6 Hz, 1H), 8.20 (s, 1H), 8.12 (d, J = 2.2 Hz, 1H), 8.08 (dd, J = 1.1, 7.0 Hz, 1H), 7.78 (d, J = 1.6 Hz, 1H), 7.69 (dd, J = 0.7, 1.9 Hz, 1H), 7.51-7.42 (m, 3H), 7.23-7.14 (m, 2H), 7.07-7.02 (m, 1H), 5.59-5.50 (m, 1H), 3.66- 3.51 (m, 8H), 3.27-3.22 (m, 2H), 2.40-2.34 (m, 5H), 2.17 (s, 3H), 1.59-1.49 (m, 4H), 1.39-1.30 (m, 2H) | 823.15 | B | |
| I-102 | 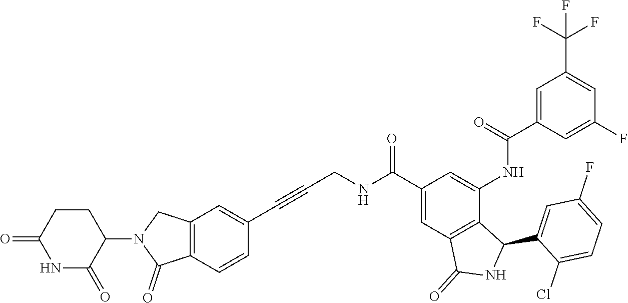 [SMILES: O=C1CCC(N2Cc3cc(C#CCNC(=O)c4cc(NC(=O)c5cc(F)cc(C(F)(F)F)c5)c5c(c4)C(=O)N[C@@H]5c4cc(F)ccc4Cl)ccc3C2=O)C(=O)N1] | 1H NMR (400 MHz, DMSO-d6) = 11.02-10.97 (m, 1H), 10.65-10.62 (m, 1H), 9.47-9.36 (m, 1H), 9.33- 9.24 (m, 1H), 8.30-8.25 (m, 1H), 8.08-8.03 (m, 1H), 7.96 (d, J = 8.2 Hz, 1H), 7.79-7.66 (m, 4H), 7.60- 7.54 (m, 1H), 7.36-7.29 (m, 1H), 7.14-7.04 (m, 1H), 6.16-5.90 (m, 1H), 5.16-5.06 (m, 1H), 4.51-4.29 (m, 4H), 2.96-2.85 (m, 1H), 2.71- 2.65 (m, 2H), 2.39 (dd, J = 4.5, 13.1 Hz, 1H), 2.04-1.96 (m, 1H) | 790.4 | A | |
| I-103 | 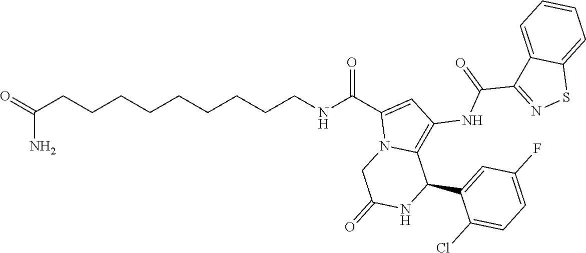 [SMILES: NC(=O)CCCCCCCCCNC(=O)c1cc(NC(=O)c2nsc3ccccc23)c2n1CC(=O)N[C@@H]2c1cc(F)ccc1Cl] | 1H NMR (400 MHz, DMSO-d6) = 9.84-9.80 (m, 1H), 8.89-8.86 (m, 1H), 8.66-8.61 (m, 1H), 8.33-8.28 (m, 1H), 8.12 (t, J = 5.6 Hz, 1H), 7.70-7.65 (m, 1H), 7.62-7.56 (m, 1H), 7.31 (dd, J = 5.1, 8.8 Hz, 1H), 7.23-7.13 (m, 2H), 7.07-7.01 (m, 2H), 6.65 (br s, 1H), 6.22 (d, J = 2.1 Hz, 1H), 5.06 (s, 2H), 3.22-3.15 (m, 2H), 2.04-1.99 (m, 2H), 1.51- 1.43 (m, 4H), 1.26 (t, J = 9.2 Hz, 10H) | 653.15 | A | |
| I-104 | 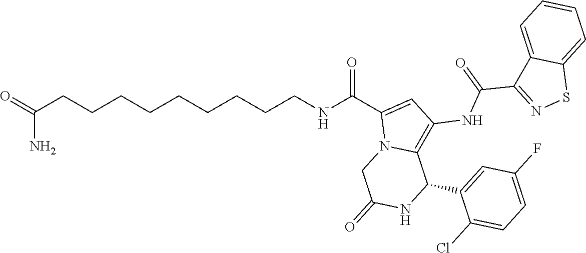 [SMILES: NC(=O)CCCCCCCCCNC(=O)c1cc(NC(=O)c2nsc3ccccc23)c2n1CC(=O)N[C@H]2c1cc(F)ccc1Cl] | 1H NMR (400 MHz, DMSO-d6) = 9.83-9.80 (m, 1H), 8.88 (d, J = 2.6 Hz, 1H), 8.67-8.61 (m, 1H), 8.30 (d, J = 8.2 Hz, 1H), 8.12 (t, J = 5.7 Hz, 1H), 7.67 (ddd, J = 1.2, 6.9, 8.2 Hz, 1H), 7.61-7.55 (m, 1H), 7.31 (dd, J = 5.1, 8.8 Hz, 1H), 7.22-7.14 (m, 2H), 7.08-7.01 (m, 2H), 6.65 (br s, 1H), 6.25-6.19 (m, 1H), 5.06 (s, 2H), 3.20 (d, J = 5.4 Hz, 2H), 2.03-1.97 (m, 2H), 1.52-1.43 (m, 4H), 1.29-1.21 (m, 10H) | 653.05 | A | |
| I-105 | 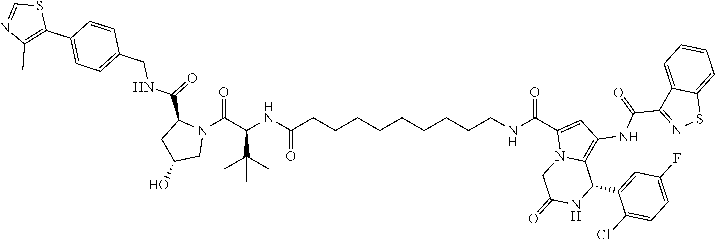 [SMILES: Cc1ncsc1-c1ccc(CNC(=O)[C@@H]2C[C@@H](O)CN2C(=O)[C@@H](NC(=O)CCCCCCCCCNC(=O)c2cc(NC(=O)c3nsc4ccccc34)c3n2CC(=O)N[C@H]3c2cc(F)ccc2Cl)C(C)(C)C)cc1] | 1H NMR (400 MHz, DMSO-d6) = 9.82 (s, 1H), 8.98 (s, 1H), 8.88 (d, J = 2.5 Hz, 1H), 8.64 (d, J = 8.2 Hz, 1H), 8.55 (t, J = 6.0 Hz, 1H), 8.30 (d, J = 8.2 Hz, 1H), 8.12 (t, J = 5.5 Hz, 1H), 7.83 (d, J = 9.4 Hz, 1H), 7.67 (dt, J = 1.1, 7.6 Hz, 1H), 7.60- 7.56 (m, 1H), 7.44-7.36 (m, 4H), 7.31 (dd, J = 5.3, 8.8 Hz, 1H), 7.16 (dd, J = 3.0, 9.2 Hz, 1H), 7.07-7.01 (m, 2H), 6.22 (s, 1H), 5.11 (d, J = 3.5 Hz, 1H), 5.06 (s, 2H), 4.54 (d, J = 9.4 Hz, 1H), 4.45-4.38 (m, 2H), 4.34 (br s, 1H), 4.23-4.18 (m, 1H), 3.65 (d, J = 4.4 Hz, 2H), 3.22-3.15 | 1065.85 | C | |
| | | (m, 2H), 2.45-2.43 (m, 3H), 2.29- | | | |
| | | 2.22 (m, 1H), 2.13-1.98 (m, 2H), | | | |
| | | 1.93-1.85 (m, 1H), 1.53-1.43 (m, | | | |
| | | 4H), 1.26 (t, J = 8.0 Hz, 10H), 0.93 | | | |
| | | (s, 9H) | | | |
| I-106 | 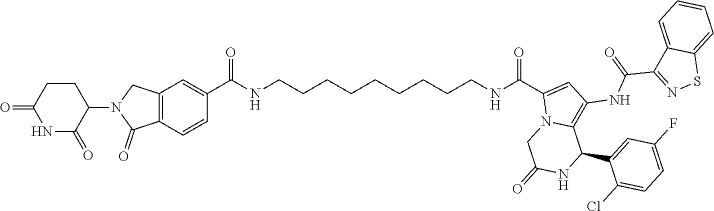 [SMILES: O=C1CCC(N2Cc3cc(C(=O)NCCCCCCCCCNC(=O)c4cc(NC(=O)c5nsc6ccccc56)c5n4CC(=O)N[C@@H]5c4cc(F)ccc4Cl)ccc3C2=O)C(=O)N1] | 1H NMR (400 MHz, DMSO-d6) = 11.00 (br s, 1H), 9.82(s, 1H), 8.89 (d, J = 2.6 Hz, 1H), 8.66-8.59 (m, 2H), 8.30 (d, J = 8.2 Hz, 1H), 8.12 (t, J = 5.6 Hz, 1H), 8.05-8.00 (m, 1H), 7.99-7.91 (m, 1H), 7.79 (d, J = 7.9 Hz, 1H), 7.67 (ddd, J = 1.1, 7.0, 8.2 Hz, 1H), 7.62-7.54 (m, 1H), 7.31 (dd, J = 5.1, 8.8 Hz, 1H), 7.16 (dd, J = 3.1, 9.1 Hz, 1H), 7.07-7.00 (m, 2H), 6.23 (s, 1H), 5.19-5.11 (m, 1H), 5.06 (s, 2H), 4.54-4.48 (m, 1H), 3.25-3.10 (m, 4H), 2.97- | 896.35 | A | |
| | | 2.85 (m, 1H), 2.64-2.57 (m, 1H), | | | |
| | | 2.44-2.35 (m, 2H), 2.06-1.98 (m, | | | |
| | | 1H), 1.58-1.44 (m, 4H), 1.30 (br s, | | | |
| | | 10H) | | | |
| I-107 | 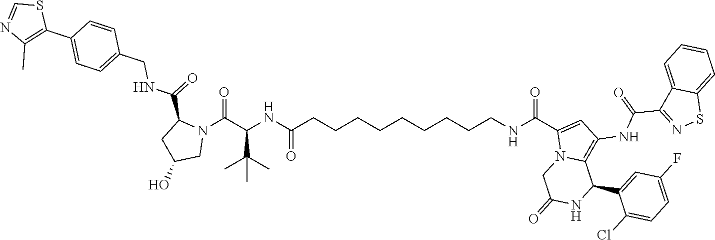 [SMILES: Cc1ncsc1-c1ccc(CNC(=O)[C@@H]2C[C@@H](O)CN2C(=O)[C@@H](NC(=O)CCCCCCCCCNC(=O)c2cc(NC(=O)c3nsc4ccccc34)c3n2CC(=O)N[C@@H]3c2cc(F)ccc2Cl)C(C)(C)C)cc1] | 1H NMR (400 MHz, DMSO-d6) = 9.82 (s, 1H), 8.98 (s, 1H), 8.88 (d, J = 2.5 Hz, 1H), 8.63 (d, J = 8.1 Hz, 1H), 8.56 (t, J = 5.9 Hz, 1H), 8.30 (d, J = 8.2 Hz, 1H), 8.12 (t, J = 5.6 Hz, 1H), 7.83 (d, J = 9.4 Hz, 1H), 7.69-7.62 (m, 1H), 7.60-7.55 (m, 1H), 7.42-7.36 (m, 4H), 7.32- 7.29 (m, 1H), 7.16 (dd, J = 3.1, 9.1 Hz, 1H), 7.07-7.01 (m, 2H), 6.22 (d, J = 1.2 Hz, 1H), 5.12 (br s, 1H), 5.06 (s, 2H), 4.54 (d, J = 9.4 Hz, 1H), 4.47-4.39 (m, 2H), 4.34 (d, J = 1.2 Hz, 1H), 4.25-4.19 (m, 1H), 3.65 (d, J = 4.8 Hz, 1H), 3.20-3.17 | 1067.8 | A | |
| | | (m, 2H), 2.44 (s, 3H), 2.25 (dd, J = | | | |
| | | 7.2, 14.2 Hz, 1H), 2.14-2.06 (m, | | | |
| | | 1H), 2.03-1.97 (m, 2H), 1.93-1.85 | | | |
| | | (m, 1H), 1.52-1.43 (m, 4H), 1.28- | | | |
| | | 1.24 (m, 10H), 0.93 (s, 9H) | | | |
| I-108 | 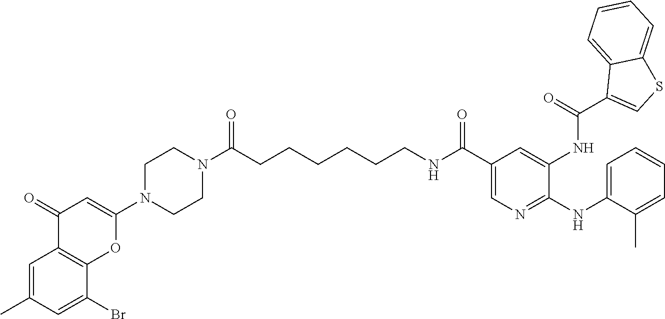 [SMILES: Cc1cc(Br)c2oc(N3CCN(C(=O)CCCCCCNC(=O)c4cnc(Nc5ccccc5C)c(NC(=O)c5csc6ccccc56)c4)CC3)cc(=O)c2c1] | 1H NMR (400 MHz, DMSO-d6) = 10.05 (s, 1H), 8.70 (s, 1H), 8.50- 8.46 (m, 1H), 8.45 (d, J = 2.2 Hz, 1H), 8.33 (t, J = 5.5 Hz, 1H), 8.22 (br s, 1H), 8.12 (d, J = 2.1 Hz, 1H), 8.11-8.07 (m, 1H), 7.80-7.76 (m, 1H), 7.70 (dd, J = 0.9, 2.1 Hz, 1H), 7.51-7.41 (m, 3H), 7.24-7.13 (m, 2H), 7.07-7.01 (m, 1H), 5.54 (s, 1H), 3.65-3.54 (m, 8H), 3.27- 3.22 (m, 2H), 2.38 (s, 3H), 2.34- 2.30 (m, 2H), 2.17 (s, 3H), 1.57- 1.47 (m, 4H), 1.37-1.29 (m, 4H) | 836.85 | B | |
| I-109 | 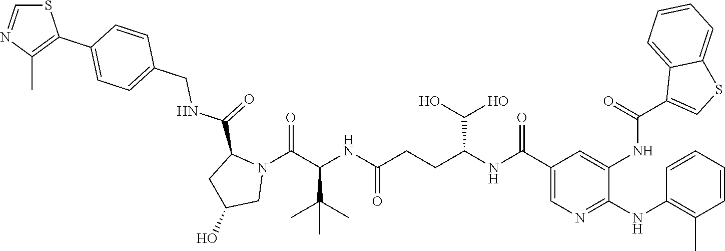 [SMILES: Cc1ccccc1Nc1ncc(C(=O)N[C@H](CCC(=O)N[C@H](C(=O)N2C[C@H](O)C[C@H]2C(=O)NCc2ccc(-c3scnc3C)cc2)C(C)(C)C)C(O)O)cc1NC(=O)c1csc2ccccc12] | 1H NMR (400 MHz, DMSO-d6) = 12.70-12.42 (m, 1H), 10.08 (s, 1H), 8.97 (s, 1H), 8.70-8.70 (m, 1H), 8.52-8.49 (m, 4H), 8.24 (s, 1H), 8.15 (d, J = 2.1 Hz, 1H), 8.10-8.08 (m, 1H), 7.99 (d, J = 9.2 Hz, 1H), 7.47-7.40 (m, 8H), 7.22-7.18 (m, 2H), 7.07-7.04 (m, 1H), 5.15-5.10 (m, 1H), 4.53 (d, J = 9.2 Hz, 1H), 4.44-4.40 (m, 2H), 4.32 (br s, 2H), 4.24-4.20 (m, 1H), 3.63 (d, J = 2.1 Hz, 2H), 2.69-2.67 (m, 3H), 2.18 (s, 4H), 2.12-2.08 (m, 1H), 2.00 (d, J = 7.8 Hz, 2H), 1.92-1.88 (m, 2H), 1.30-1.20 (m, 9H) | 945.1 | D | |
| I-110 | 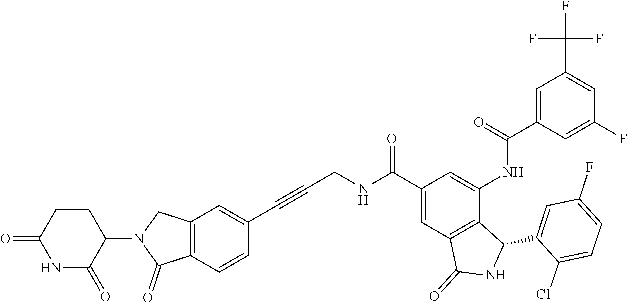 [SMILES: O=C1CCC(N2Cc3cc(C#CCNC(=O)c4cc(NC(=O)c5cc(F)cc(C(F)(F)F)c5)c5c(c4)C(=O)N[C@H]5c4cc(F)ccc4Cl)ccc3C2=O)C(=O)N1] | 1H NMR (400 MHz, DMSO-d6) = 11.01-10.98 (m, 1H), 10.64 (s, 1H), 9.46-9.36 (m, 1H), 9.32-9.22 (m, 1H), 8.30-8.25 (m, 1H), 8.21- 8.04 (m, 1H), 7.96 (d, J = 8.5 Hz, 1H), 7.82-7.62 (m, 5H), 7.61-7.54 (m, 1H), 7.36-7.29 (m, 1H), 7.15- 7.05 (m, 1H), 6.13-5.92 (m, 1H), 5.16-5.06 (m, 1H), 4.49-4.30 (m, 4H), 2.98-2.85 (m, 1H), 2.64-2.56 (m, 1H), 2.39 (dd, J = 4.4, 13.1 Hz, 1H), 2.06-1.95 (m, 1H) | 790.35 | A | |
| I-111 | 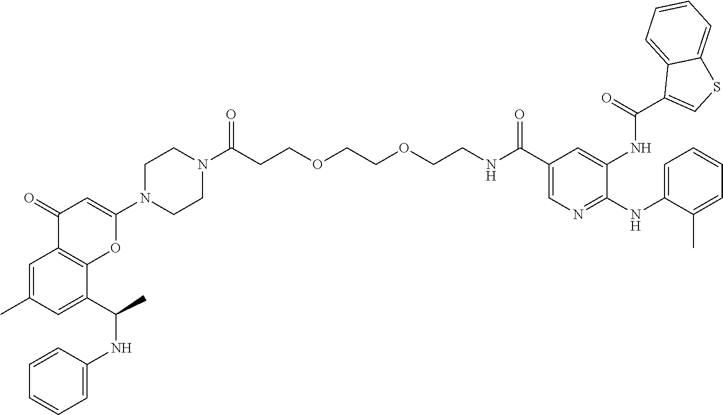 [SMILES: Cc1cc([C@@H](C)Nc2ccccc2)c2oc(N3CCN(C(=O)CCOCCOCCNC(=O)c4cnc(Nc5ccccc5C)c(NC(=O)c5csc6ccccc56)c4)CC3)cc(=O)c2c1] | 1H NMR (400 MHz, DMSO-d6) = 10.06-10.03 (m, 1H), 8.71-8.69 (m, 1H), 8.50-8.44 (m, 2H), 8.42- 8.38 (m, 1H), 8.24-8.19 (m, 1H), 8.15-8.11 (m, 1H), 8.10-8.05 (m, 1H), 7.57 (d, J = 1.3 Hz, 1H), 7.51- 7.42 (m, 4H), 7.24-7.14 (m, 2H), 7.09-6.96 (m, 3H), 6.51-6.44 (m, 3H), 6.24-6.20 (m, 1H), 5.51 (s, 1H), 4.97-4.89 (m, 1H), 3.66-3.51 (m, 16H), 3.41-3.38 (m, 2H), 2.62- 2.58 (m, 2H), 2.29 (s, 3H), 2.17 (s, 3H), 1.48 (d, J = 6.6 Hz, 3H) | 908.25 | A | |
| I-112 | 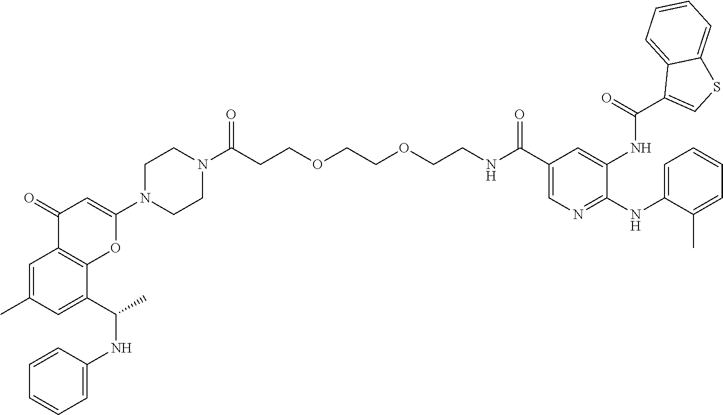 [SMILES: Cc1cc([C@H](C)Nc2ccccc2)c2oc(N3CCN(C(=O)CCOCCOCCNC(=O)c4cnc(Nc5ccccc5C)c(NC(=O)c5csc6ccccc56)c4)CC3)cc(=O)c2c1] | 1H NMR (400 MHz, DMSO-d6) = 10.06-10.04 (m, 1H), 8.73-8.68 (m, 1H), 8.50-8.44 (m, 2H), 8.43- 8.39 (m, 1H), 8.23-8.19 (m, 1H), 8.13 (d, J = 2.1 Hz, 1H), 8.10-8.06 (m, 1H), 7.60-7.55 (m, 1H), 7.48- 7.41 (m, 4H), 7.23-7.15 (m, 2H), 7.06-6.97 (m, 3H), 6.49-6.44 (m, 3H), 6.25-6.19 (m, 1H), 5.53-5.50 (m, 1H), 4.97-4.91 (m, 1H), 3.66- 3.58 (m, 8H), 3.52 (d,J = 1.5 Hz, 8H), 3.42-3.38 (m, 2H), 2.60 (s, 2H), 2.29 (s, 3H), 2.17 (s, 3H), 1.48 (d, J = 6.7 Hz, 3H) | 908.25 | D | |
| I-113 | 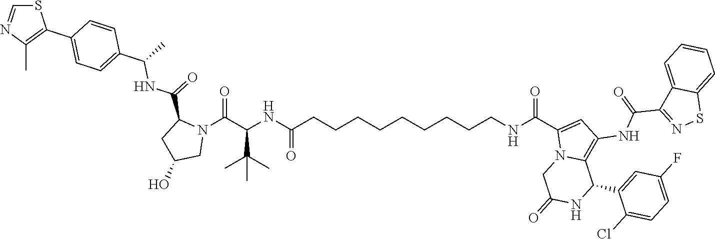 [SMILES: Cc1ncsc1-c1ccc([C@H](C)NC(=O)[C@@H]2C[C@@H](O)CN2C(=O)[C@@H](NC(=O)CCCCCCCCCNC(=O)c2cc(NC(=O)c3nsc4ccccc34)c3n2CC(=O)N[C@H]3c2cc(F)ccc2Cl)C(C)(C)C)cc1] | 1H NMR (400 MHz, DMSO-d6) = 9.83-9.81 (m, 1H), 8.98-8.96 (m, 1H), 8.88 (d, J = 2.5 Hz, 1H), 8.66- 8.62 (m, 1H), 8.36 (d, J = 7.9 Hz, 1H), 8.32-8.28 (m, 1H), 8.14-8.10 (m, 1H), 7.80-7.75 (m, 1H), 7.70- 7.65 (m, 1H), 7.61-7.56 (m, 1H), 7.45-7.41 (m, 2H), 7.39-7.35 (m, 2H), 7.33-7.28 (m, 1H), 7.19-7.14 (m, 1H), 7.08-7.03 (m, 2H), 6.24- 6.21 (m, 1H), 5.34-5.30 (m, 1H), 5.10-5.05 (m, 3H), 4.93-4.88 (m, 1H), 4.53-4.49 (m, 1H), 4.42 (t, J = 7.9 Hz, 1H), 4.27 (d, J = 3.2 Hz, 1H), 3.60-3.59 (m, 1H), 3.21-3.17 | 1080.2 | B | |
| | | (m, 2H), 2.28-2.21 (m, 1H), 2.14- | | | |
| | | 2.07 (m, 1H), 2.03-1.97 (m, 3H), | | | |
| | | 1.82-1.75 (m, 1H), 1.51-1.46 (m, | | | |
| | | 4H), 1.37 (d, J = 6.9 Hz, 3H), 1.23 | | | |
| | | (br s, 11H), 0.93 (s, 9H) | | | |
| I-114 | 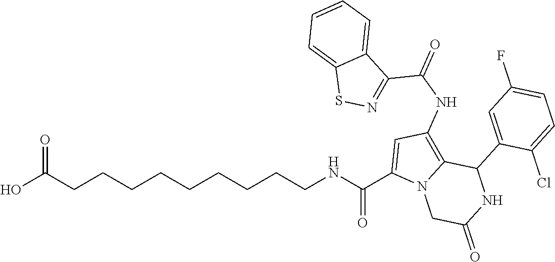 [SMILES: O=C(O)CCCCCCCCCNC(=O)c1cc(NC(=O)c2nsc3ccccc23)c2n1CC(=O)NC2c1cc(F)ccc1Cl] | 1H NMR (400 MHz, DMSO-d6) = 11.94 (br s, 1H), 9.82 (s, 1H), 8.88 (d, J = 2.5 Hz, 1H), 8.64 (d, J = 8.1 Hz, 1H), 8.30 (d, J = 8.2 Hz, 1H), 8.12 (t, J = 5.6 Hz, 1H), 7.71-7.66 (m, 1H), 7.59 (ddd, J = 1.0, 7.0, 8.1 Hz, 1H), 7.32-7.28 (m, 1H), 7.18-7.15 (m, 1H), 7.07 (s, 1H), 7.05-7.01 (m, 1H), 6.22 (s, 1H), 5.06 (s, 2H), 3.23-3.14 (m, 2H), 2.19 (t, J = 7.3 Hz, 2H), 1.49 (d, J = 2.9 Hz, 4H), 1.27 (br s, 10H) | 654 | | |
| I-115 | 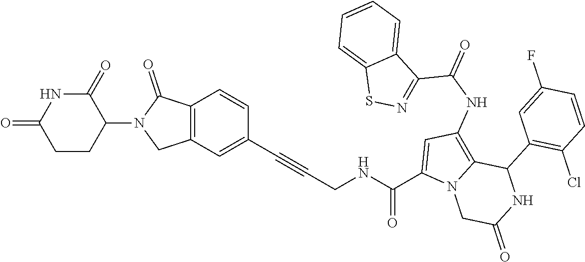 [SMILES: O=C1CCC(N2Cc3cc(C#CCNC(=O)c4cc(NC(=O)c5nsc6ccccc56)c5n4CC(=O)NC5c4cc(F)ccc4Cl)ccc3C2=O)C(=O)N1] | 1H NMR (400 MHz, DMSO-d6) = 10.99 (s, 1H), 9.87 (s, 1H), 8.91 (d, J = 2.5 Hz, 1H), 8.74 (t, J = 5.6 Hz, 1H), 8.64 (d, J = 8.1 Hz, 1H), 8.31 (d, J = 8.2 Hz, 1H), 7.75-7.64 (m, 3H), 7.63-7.54 (m, 2H), 7.31 (dd, J = 5.1, 8.8 Hz, 1H), 7.23-7.14 (m, 2H), 7.04 (dt, J = 3.0, 8.4 Hz, 1H), 6.24 (s, 1H), 5.16-5.06 (m, 3H), 4.49-4.26 (m, 4H), 2.96-2.85 (m, 1H), 2.61 (d, J = 2.9 Hz, 1H), 2.39 (dd, J = 4.7, 13.2 Hz, 1H), 2.04-1.96 (m, 2H) | 761.9 | | |
| I-116 | 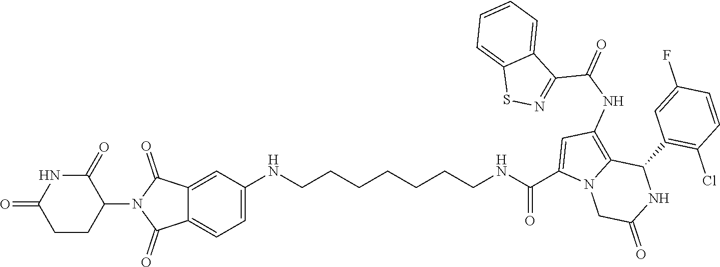 [SMILES: O=C1CCC(N2C(=O)c3ccc(NCCCCCCCNC(=O)c4cc(NC(=O)c5nsc6ccccc56)c5n4CC(=O)N[C@H]5c4cc(F)ccc4Cl)cc3C2=O)C(=O)N1] | 1H NMR (400 MHz, DMSO-d6) = 11.05 (br s, 1H), 9.82 (s, 1H), 8.90- 8.86 (m, 1H), 8.63 (dd, J = 0.9, 8.1 Hz, 1H), 8.35-8.27 (m, 1H), 8.17- 8.09 (m, 1H), 7.71-7.64 (m, 1H), 7.62-7.52 (m, 2H), 7.34-7.29 (m, 1H), 7.18-7.13 (m, 1H), 7.11-7.00 (m, 3H), 6.96-6.92 (m, 1H), 6.84 (dd, J = 2.1, 8.5 Hz, 1H), 6.22 (d, J = 1.2 Hz, 1H), 5.09-4.97 (m, 3H), 3.25-3.13 (m, 4H), 2.92-2.81 (m, 1H), 2.55 (br s, 2H), 2.01-1.96 (m, 1H), 1.60-1.49 (m, 4H), 1.41- 1.31 (m, 6H) | 853.15 | | |
| I-117 | 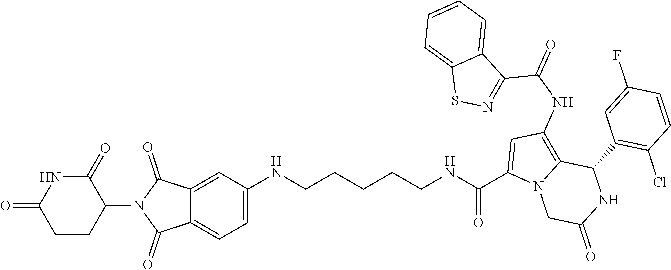 [SMILES: O=C1CCC(N2C(=O)c3ccc(NCCCCCNC(=O)c4cc(NC(=O)c5nsc6ccccc56)c5n4CC(=O)N[C@H]5c4cc(F)ccc4Cl)cc3C2=O)C(=O)N1] | 1H NMR (400 MHz, DMSO-d6) = 11.05 (br s, 1H), 9.84-9.80 (m, 1H), 8.89-8.86 (m, 1H), 8.63 (d, J = 8.2 Hz, 1H), 8.33-8.28 (m, 1H), 8.15 (t, J = 5.5 Hz, 1H), 7.70-7.64 (m, 1H), 7.63-7.52 (m, 2H), 7.33-7.28 (m, 1H), 7.19-7.09 (m, 2H), 7.08- 6.99 (m, 2H), 6.93 (s, 1H), 6.88- 6.83 (m, 1H), 6.25-6.19 (m, 1H), 5.09-4.97 (m, 3H), 3.25-3.16 (m, 4H), 2.94-2.79 (m, 1H), 2.62-2.56 (m, 2H), 2.04-1.94 (m, 1H), 1.68- 1.52 (m, 4H), 1.47-1.38 (m, 2H) | 825.05 | | |
| I-118 | 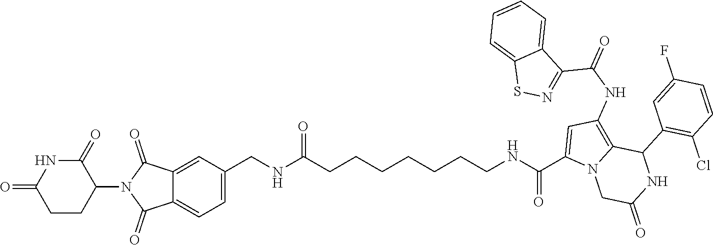 [SMILES: O=C(CCCCCCCNC(=O)c1cc(NC(=O)c2nsc3ccccc23)c2n1CC(=O)NC2c1cc(F)ccc1Cl)NCc1ccc2c(c1)C(=O)N(C1CCC(=O)NC1=O)C2=O] | 1H NMR (400 MHz, DMSO-d6) = 10.97 (s, 1H), 9.82 (s, 1H), 8.89 (d, J = 2.5 Hz, 1H), 8.63 (d, J = 8.2 Hz, 1H), 8.40 (t, J = 6.0 Hz, 1H), 8.30 (d, J = 8.2 Hz, 1H), 8.13 (t, J = 5.6 Hz, 1H), 7.71-7.64 (m, 2H), 7.62-7.57 (m, 1H), 7.45 (s, 1H), 7.38 (d, J = 8.1 Hz, 1H), 7.31 (dd, J = 5.1, 8.9 Hz, 1H), 7.16 (dd, J = 3.0, 9.2 Hz, 1H), 7.07-7.04 (m, 1H), 6.22 (s, 1H), 5.13-5.04 (m, 3H), 4.47-4.41 (m, 1H), 4.36 (d, J = 5.9 Hz, 2H), 4.33-4.25 (m, 1H), 3.28 (br s, 1H), 3.24-3.16 (m, 2H), 2.97-2.85 (m, 1H), 2.59 (d, | 381.05 | | |
| | | J = 18.6 Hz, 1H), 2.39 (br dd, J = | | | |
| | | 4.4, 12.9 Hz, 1H), 2.16 (t, J = 7.4 | | | |
| | | Hz, 2H), 2.03-1.96 (m, 1H), 1.59- | | | |
| | | 1.46 (m, 4H), 1.33-1.25 (m, 6H) | | | |
| I-119 | 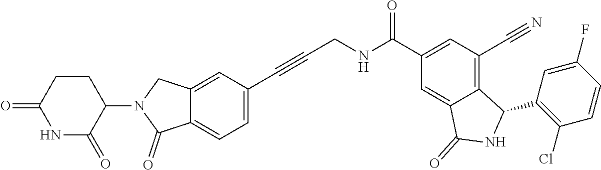 [SMILES: N#Cc1cc(C(=O)NCC#Cc2ccc3c(c2)CN(C2CCC(=O)NC2=O)C3=O)cc2c1[C@H](c1cc(F)ccc1Cl)NC2=O] | | | | |
| I-120 | 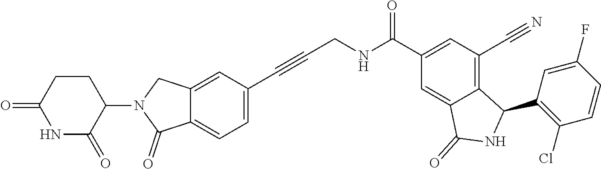 [SMILES: N#Cc1cc(C(=O)NCC#Cc2ccc3c(c2)CN(C2CCC(=O)NC2=O)C3=O)cc2c1[C@@H](c1cc(F)ccc1Cl)NC2=O] | | | | |
| I-121 | 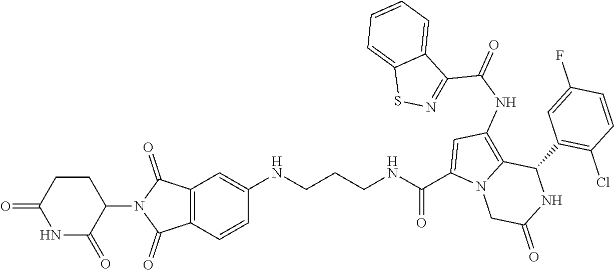 [SMILES: O=C1CCC(N2C(=O)c3ccc(NCCCNC(=O)c4cc(NC(=O)c5nsc6ccccc56)c5n4CC(=O)N[C@H]5c4cc(F)ccc4Cl)cc3C2=O)C(=O)N1] | | | | |
| I-122 | 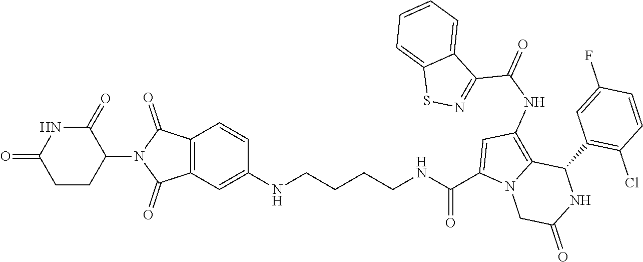 [SMILES: O=C1CCC(N2C(=O)c3ccc(NCCCCNC(=O)c4cc(NC(=O)c5nsc6ccccc56)c5n4CC(=O)N[C@H]5c4cc(F)ccc4Cl)cc3C2=O)C(=O)N1] | | | | |
| I-123 | 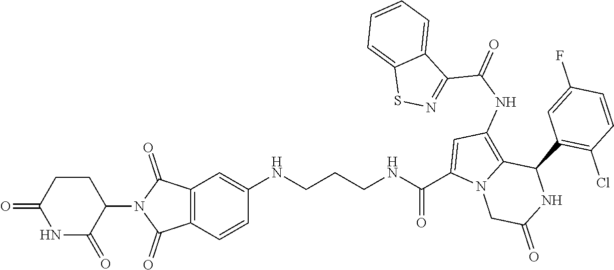 [SMILES: O=C1CCC(N2C(=O)c3ccc(NCCCNC(=O)c4cc(NC(=O)c5nsc6ccccc56)c5n4CC(=O)N[C@@H]5c4cc(F)ccc4Cl)cc3C2=O)C(=O)N1] | | | | |
| I-124 | 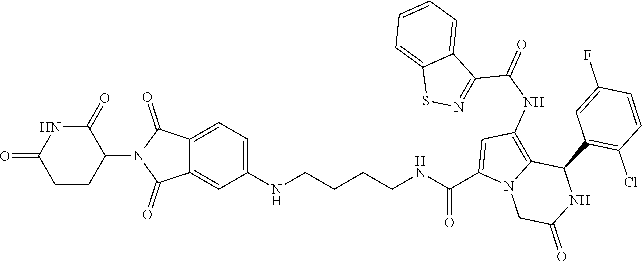 [SMILES: O=C1CCC(N2C(=O)c3ccc(NCCCCNC(=O)c4cc(NC(=O)c5nsc6ccccc56)c5n4CC(=O)N[C@@H]5c4cc(F)ccc4Cl)cc3C2=O)C(=O)N1] | | | | |
| I-125 | 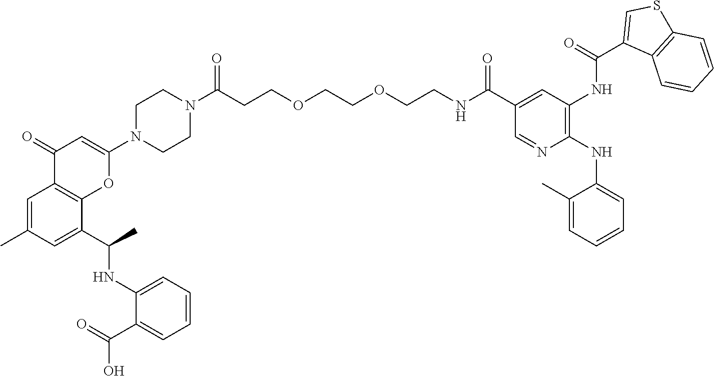 [SMILES: Cc1cc([C@@H](C)Nc2ccccc2C(=O)O)c2oc(N3CCN(C(=O)CCOCCOCCNC(=O)c4cnc(Nc5ccccc5C)c(NC(=O)c5csc6ccccc56)c4)CC3)cc(=O)c2c1] | (400 MHz, DMSO-d6) = 12.77 (br s, 1H), 10.05 (br s, 1H), 8.70 (s, 1H), 8.47-8.51 (m, 1H), 8.45 (d, J = 2.1 Hz, 1H), 8.40 (t, J = 5.7 Hz, 1H), 8.35 (br s, 1H), 8.21 (s, 1H), 8.13 (d, J = 2.2 Hz, 1H), 8.07-8.09 (m, 1H), 7.81 (dd, J = 7.9, 1.6 Hz, 1H), 7.58-7.61 (m, 1H), 7.42-7.49 (m, 3H), 7.37 (d, J = 2.1 Hz, 1H), 7.14-7.25 (m, 3H), 7.05 (dd, J = 7.4, 1.2 Hz, 1H), 6.54 (t, J = 7.6 Hz, 1H), 6.46 (d, J = 8.8 Hz, 1H), 5.52 (s, 1H), 5.09 (t, J = 6.5 Hz, 1H), 3.57-3.67 (m, 8H), 3.50-3.56 (m, 8H), 3.37-3.43 (m, 2H), 2.59- 2.62 (m, 2H), 2.30 (s, 3H), 2.16 (s, 3H), 1.96-2.02 (m, 1H), 1.57 ppm (d, J = 6.6 Hz, 3H) | 952.25 | A | |
| I-126 | 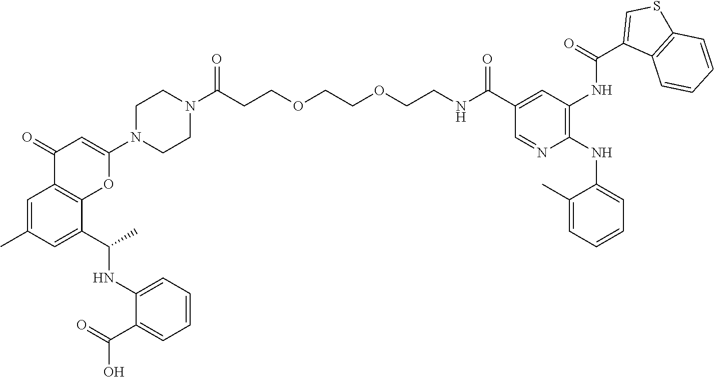 [SMILES: Cc1cc([C@H](C)Nc2ccccc2C(=O)O)c2oc(N3CCN(C(=O)CCOCCOCCNC(=O)c4cnc(Nc5ccccc5C)c(NC(=O)c5csc6ccccc56)c4)CC3)cc(=O)c2c1] | (400 MHz, DMSO-d6): = 12.77 (br s, 1H), 10.06 (br s, 1H), 8.71 (br s, 1H), 8.46-8.51 (m, 1H), 8.45 (d, J = 2.1 Hz, 1H), 8.40 (t, J = 5.5 Hz, 1H), 8.19-8.25 (m, 1H), 8.13 (d, J = 1.6 Hz, 1H), 8.08 (dd, J = 6.9, 1.2 Hz, 1H), 7.80 (dd, J = 7.9, 1.6 Hz, 1H), 7.60 (d, J = 1.5 Hz, 1H), 7.4I-7.51 (m, 3H), 7.38 (d, J = 1.8 Hz, 1H), 7.13-7.25 (m, 3H), 7.01-7.07 (m, 1H), 6.53 (t, J = 7.3 Hz, 1H), 6.45 (d, J = 8.2 Hz, 1H), 5.51 (s, 1H), 5.05-5.13 (m, 1H), 3.64 (t, J = 6.6 Hz, 2H), 3.59 (br s, 6H), 3.50-3.55 (m, 8H), 3.40-3.43 (m, 2H), 2.60 (br s, 2H), 2.30 (s, 3H), 2.16 (s, 3H), 1.95- 2.04 (m, 1H), 1.57 ppm (d, J = 6.6 Hz, 3H) | 952.3 | B | |
| I-127 | 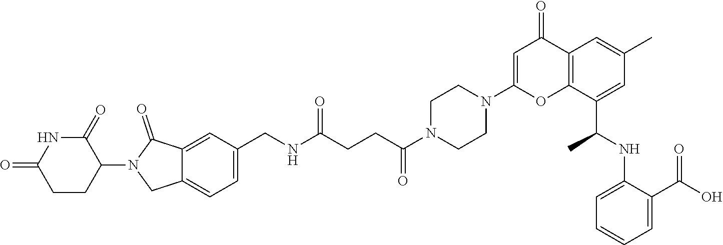 [SMILES: Cc1cc([C@H](C)Nc2ccccc2C(=O)O)c2oc(N3CCN(C(=O)CCC(=O)NCc4ccc5c(c4)C(=O)N(C4CCC(=O)NC4=O)C5)CC3)cc(=O)c2c1] | (400 MHz, CD₃SOCD₃, 298K) (ppm) = 12.78 (br s, 1H), 10.97 (s, 1H), 8.46 (s, 1H), 8.35 (d, J = 5.9 Hz, 1H), 7.81 (dd, J = 1.6, 7.9 Hz, 1H), 7.62 (d, J = 4.0 Hz, 2H), 7.54-7.51 (m, 2H), 7.38 (d, J = 2.1 Hz, 1H), 7.25 (t, J = 7.4 Hz, 1H), 6.55 (t, J = 7.6 Hz, 1H), 6.47 (d, J = 8.4 Hz, 1H), 5.53 (s, 1H), 5.14- 5.04 (m, 2H), 4.46-4.23 (m, 4H), 3.64 (br s, 7H), 3.58-3.52 (m, 2H), 2.95-2.84 (m, 1H), 2.62 (d, J = 6.6 Hz, 2H), 2.58-2.56 (m, 1H), 2.42- 2.35 (m, 1H), 2.31 (s, 3H), 2.03- 1.94 (m, 2H), 1.59 (d, J = 6.7 Hz, | 763.3 | D | |
| | | 3H) | | | |
| I-128 | 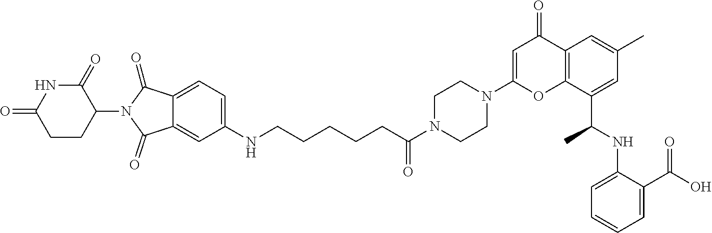 [SMILES: Cc1cc([C@H](C)Nc2ccccc2C(=O)O)c2oc(N3CCN(C(=O)CCCCCNc4ccc5c(c4)C(=O)N(C4CCC(=O)NC4=O)C5=O)CC3)cc(=O)c2c1] | 1H NMR (400 MHz, DMSO-d6) = 12.77 (br s, 1H), 11.05(s, 1H), 8.35 (d, J = 5.7 Hz, 1H), 7.81 (dd, J = 1.6, 7.9 Hz, 1H), 7.61 (d, J = 1.5 Hz, 1H), 7.55 (d, J = 8.4 Hz, 1H), 7.38 (d, J = 2.1 Hz, 1H), 7.29-7.21 (m, 1H), 7.11 (t, J = 5.4 Hz, 1H), 6.94 (d, J = 1.8 Hz, 1H), 6.84 (dd, J = 2.0, 8.4 Hz, 1H), 6.58-6.52 (m, 1H), 6.47 (d, J = 8.4 Hz, 1H), 5.53 (s, 1H), 5.10 (t, J = 6.6 Hz, 1H), 5.02 (dd, J = 5.4, 12.9 Hz, 1H), 3.64-3.51 (m, 8H), 3.20-3.12 (m, 2H), 2.92-2.80 (m, 1H), 2.58 (d, | 777.45 | D | |
| | | J = 3.1 Hz, 1H), 2.37 (t, J = 7.3 | | | |
| | | Hz, 2H), 2.31 (s, 3H), 2.04-1.93 | | | |
| | | (m, 2H), 1.65-1.51 (m, 7H), 1.44- | | | |
| | | 1.36 (m, 2H) | | | |
| I-129 | 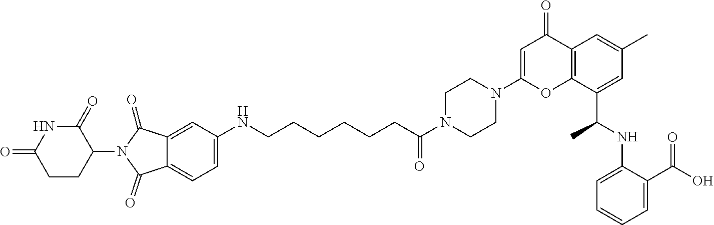 [SMILES: Cc1cc([C@H](C)Nc2ccccc2C(=O)O)c2oc(N3CCN(C(=O)CCCCCCNc4ccc5c(c4)C(=O)N(C4CCC(=O)NC4=O)C5=O)CC3)cc(=O)c2c1] | 1H NMR (400 MHz, DMSO-d6, 25C): = 12.75 (br s, 1H), 11.05 (s, 1H), 8.35 (d, J = 6.2 Hz, 1H), 7.81 (dd, J = 7.9, 1.6 Hz, 1H), 7.60- 7.63 (m, 1H), 7.55 (d, J = 8.4 Hz, 1H), 7.38 (d, J = 2.1 Hz, 1H), 7.22-7.27 (m, 1H), 7.10 (t, J = 5.4 Hz, 1H), 6.94 (d, J = 1.8 Hz, 1H), 6.84 (dd, J = 8.4, 2.0 Hz, 1H), 6.53-6.58 (m, 1H), 6.47 (d, J = 8.5 Hz, 1H), 5.54 (s, 1H), 5.11 (t, J = 6.5 Hz, 1H), 5.02 (dd, J = 12.8, 5.4 Hz, 1H), 3.50-3.68 (m, 8H), 3.11- 3.19 (m, 2H), 2.79-2.93 (m, 1H), | 791.5 | D | |
| | | 2.54-2.61 (m, 1H), 2.32-2.38 (m, | | | |
| | | 2H), 2.31 (s, 3H), 1.95-2.03 (m, | | | |
| | | 1H), 1.49-1.63 (m, 7H), 1.31-1.43 | | | |
| | | ppm (m, 4H) | | | |
| I-130 | 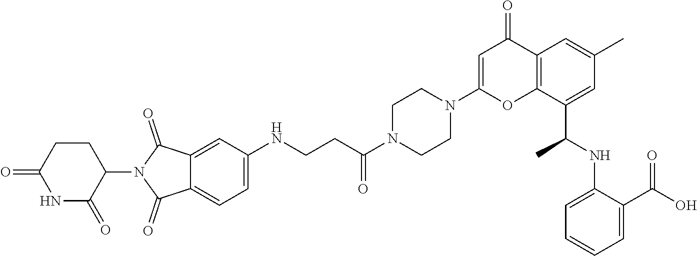 [SMILES: Cc1cc([C@H](C)Nc2ccccc2C(=O)O)c2oc(N3CCN(C(=O)CCNc4ccc5c(c4)C(=O)N(C4CCC(=O)NC4=O)C5=O)CC3)cc(=O)c2c1] | 1H NMR (400 MHz, DMSO-d6, 25C): = 12.77 (br s, 1H), 11.05 (s, 1H), 8.34 (d, J = 6.0 Hz, 1H), 7.81 (dd, J = 8.0, 1.5 Hz, 1H), 7.61 (d, J = 1.3 Hz, 1H), 7.57 (d, J = 8.4 Hz, 1H), 7.38 (d, J = 2.1 Hz, 1H), 7.28-7.21 (m, 1H), 7.14 (t, J = 5.6 Hz, 1H), 6.98 (d, J = 1.8 Hz, 1H), 6.88 (dd, J = 8.4, 2.0 Hz, 1H), 6.55 (t, J = 7.6 Hz, 1H), 6.46 (d, J = 8.5 Hz, 1H), 5.54 (s, 1H), 5.14-5.07 (m, 1H), 5.03 (dd, J = 12.9, 5.4 Hz, 1H), 3.67-3.54 (m, 8H), 3.49-3.45 (m, 2H), 3.01 (td, J = 6.5, 4.0 Hz, 1H), 2.92-2.82 (m, 1H), 2.69-2.67 (m, 1H), 2.62-2.55 (m, 1H), 2.30 | 735.2 | D | |
| | | (s, 3H), 2.03-1.95 (m, 2H), 1.58 (d, | | | |
| | | J = 6.7 Hz, 3H) | | | |
| I-131 | 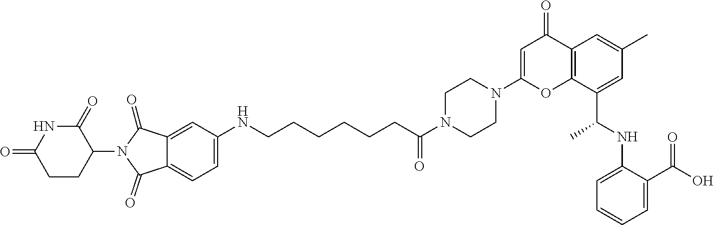 [SMILES: Cc1cc([C@@H](C)Nc2ccccc2C(=O)O)c2oc(N3CCN(C(=O)CCCCCCNc4ccc5c(c4)C(=O)N(C4CCC(=O)NC4=O)C5=O)CC3)cc(=O)c2c1] | 1H NMR (400 MHz, DMSO-d6, 25C): = 12.77 (br s, 1H), 11.09 (s, 1H), 8.35 (d, J = 6.5 Hz, 1H), 7.81 (dd, J = 7.8, 1.7 Hz, 1H), 7.56-7.65 (m, 2H), 7.38 (d, J = 2.1 Hz, 1H), 7.11-7.31 (m, 2H), 7.03 (d, J = 7.0 Hz, 1H), 6.79 (t, J = 6.3 Hz, 1H), 6.55 (t, J = 7.2 Hz, 1H), 6.46 (d, J = 8.4 Hz, 1H), 5.53 (s, 1H), 4.94- 5.18 (m, 2H), 3.51-3.71 (m, 10H), 2.82-2.93 (m, 1H), 2.72 (t, J = 5.9 Hz, 2H), 2.59 (br s, 1H), 2.30 (s, 3H), 1.95-2.03 (m, 2H), 1.58 ppm (d, J = 6.6 Hz, 3H) | 735.15 | B | |
| I-132 | 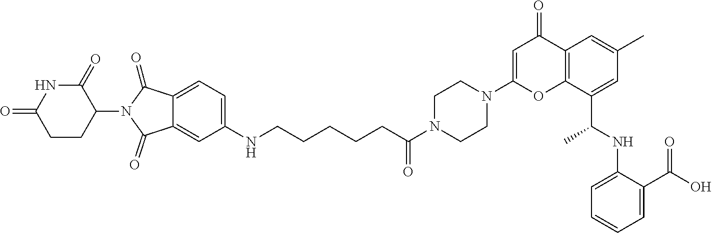 [SMILES: Cc1cc([C@@H](C)Nc2ccccc2C(=O)O)c2oc(N3CCN(C(=O)CCCCCNc4ccc5c(c4)C(=O)N(C4CCC(=O)NC4=O)C5=O)CC3)cc(=O)c2c1] | 1H NMR (400 MHz, DMSO-d6) = 12.77 (br s, 1H), 11.05 (s, 1H), 8.35 (d, J = 5.6 Hz, 1H), 7.81 (dd, J = 1.6, 7.9 Hz, 1H), 7.62-7.60 (m, 1H), 7.55 (d, J = 8.4 Hz, 1H), 7.38 (d, J = 2.2 Hz, 1H), 7.27-7.22 (m, 1H), 7.11 (t, J = 5.1 Hz, 1H), 6.94 (d, J = 1.8 Hz, 1H), 6.84 (dd, J = 2.0, 8.4 Hz, 1H), 6.55 (t, J = 7.6 Hz, 1H), 6.47 (d, J = 8.4 Hz, 1H), 5.53 (s, 1H), 5.10 (t, J = 6.4 Hz, 1H), 5.02 (dd, J = 5.4, 12.8 Hz, 1H), 3.64-3.52 (m, 8H), 3.16 (q, J = 6.5 Hz, 2H), 2.92-2.82 (m, 1H), 2.58 (d, J = 2.5 Hz, 1H), 2.37 (t, | 775.3 | C | |
| | | J = 7.3 Hz, 2H), 2.31 (s, 3H), 2.02- | | | |
| | | 1.93 (m, 2H), 1.63-1.53 (m, 7H), | | | |
| | | 1.45-1.34 (m, 2H) | | | |
| I-133 | 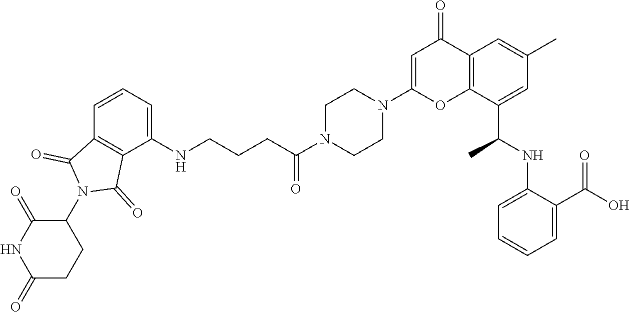 [SMILES: Cc1cc([C@H](C)Nc2ccccc2C(=O)O)c2oc(N3CCN(C(=O)CCCNc4cccc5c4C(=O)N(C4CCC(=O)NC4=O)C5=O)CC3)cc(=O)c2c1] | 1H NMR (400 MHz, DMSO-d6, 25C): = 12.77 (br s, 1H), 11.09 (s, 1H), 8.35 (br s, 1H), 7.81 (dd, J = 7.9, 1.6 Hz, 1H), 7.61 (d, J = 2.3 Hz, 1H), 7.55-7.60 (m, 1H), 7.38 (d, J = 2.1 Hz, 1H), 7.28-7.21 (m, 1H), 7.18 (d, J = 8.5 Hz, 1H), 7.02 (d, J = 6.9 Hz, 1H), 6.64-6.71 (m, 1H), 6.58-6.52 (m, 1H), 6.46 (d, J = 8.2 Hz, 1H), 5.54 (s, 1H), 5.14- 5.08 (m, 1H), 5.05 (dd, J = 12.7, 5.4 Hz, 1H), 3.66-3.53 (m, 8H), 3.30-3.24 (m, 2H), 2.93-2.81 (m, 1H), 2.63-2.59 (m, 1H), 2.46-2.43 (m, 2H), 2.31 (s, 3H), 2.05-1.94 (m, 2H), 1.82 (t, J = 7.3 Hz, 2H), 1.59 (d, J = 6.6 Hz, 3H) | 749.15 | D | |
| I-134 | 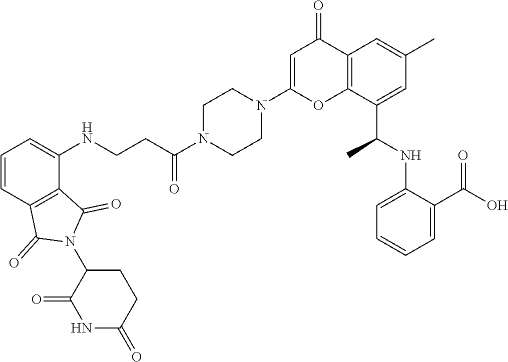 [SMILES: Cc1cc([C@H](C)Nc2ccccc2C(=O)O)c2oc(N3CCN(C(=O)CCNc4cccc5c4C(=O)N(C4CCC(=O)NC4=O)C5=O)CC3)cc(=O)c2c1] | 1H NMR (400 MHz, DMSO-d6, 25C): = 12.77 (br s, 1H), 11.09 (s, 1H), 8.35 (d, J = 6.5 Hz, 1H), 7.81 (dd, J = 7.8, 1.7 Hz, 1H), 7.56-7.65 (m, 2H), 7.38 (d, J = 2.1 Hz, 1H), 7.11-7.31 (m, 2H), 7.03 (d, J = 7.0 Hz, 1H), 6.79 (t, J = 6.3 Hz, 1H), 6.55 (t, J = 7.2 Hz, 1H), 6.46 (d, J = 8.4 Hz, 1H), 5.53 (s, 1H), 4.94- 5.18 (m, 2H), 3.51-3.71 (m, 10H), 2.82-2.93 (m, 1H), 2.72 (t, J = 5.9 Hz, 2H), 2.59 (br s, 1H), 2.30 (s, 3H), 1.95-2.03 (m, 2H), 1.58 ppm (d, J = 6.6 Hz, 3H) | 735.15 | D | |
| I-135 | 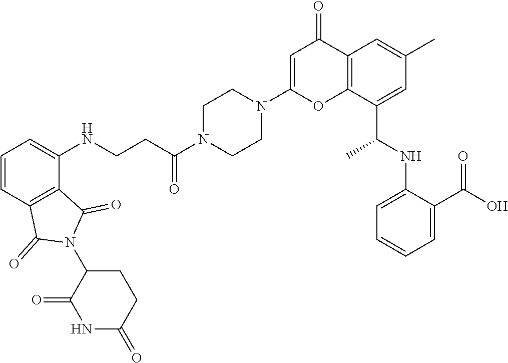 [SMILES: Cc1cc([C@@H](C)Nc2ccccc2C(=O)O)c2oc(N3CCN(C(=O)CCNc4cccc5c4C(=O)N(C4CCC(=O)NC4=O)C5=O)CC3)cc(=O)c2c1] | 1H NMR (400 MHz, DMSO-d6, 25C): = 12.77 (s, 1H), 11.09 (s, 1H), 8.35 (d, J = 6.5 Hz, 1H), 7.81 (dd, J = 7.9, 1.6 Hz, 1H), 7.54-7.64 (m, 2H), 7.38 (d, J = 2.2 Hz, 1H), 7.21-7.27 (m, 1H), 7.17 (d, J = 8.7 Hz, 1H), 7.03 (d, J = 7.0 Hz, 1H), 6.79 (t, J = 6.3 Hz, 1H), 6.52-6.58 (m, 1H), 6.46 (d, J = 8.5 Hz, 1H), 5.53 (s, 1H), 4.99-5.13 (m, 2H), 3.52-3.69 (m, 10H), 2.82-2.91 (m, 1H), 2.68-2.74 (m, 2H), 2.59 (d, J = 1.9 Hz, 1H), 2.30 (s, 3H), 1.94- 2.04 (m, 2H), 1.58 ppm (d, J = 6.6 Hz, 3H) | 735.15 | C | |
| I-136 | 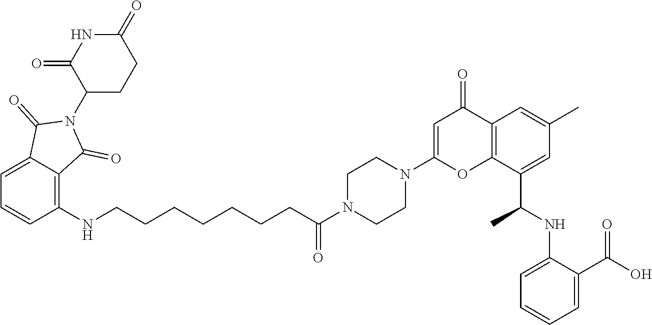 [SMILES: Cc1cc([C@H](C)Nc2ccccc2C(=O)O)c2oc(N3CCN(C(=O)CCCCCCCNc4cccc5c4C(=O)N(C4CCC(=O)NC4=O)C5=O)CC3)cc(=O)c2c1] | 1H NMR (400 MHz, CD₃SOCD₃, 298K)(ppm) = 12.76(br s, 1H), 11.09 (s, 1H), 8.34 (br s, 1H), 7.81 (dd, J = 1.5, 8.0 Hz, 1H), 7.66-7.49 (m, 2H), 7.38 (d, J = 1.9 Hz, 1H), 7.24 (t, J = 8.1 Hz, 1H), 7.09 (d, J = 8.5 Hz, 1H), 7.01 (d, J = 7.0 Hz, 1H), 6.59-6.51 (m, 2H), 6.47 (d, J = 8.5 Hz, 1H), 5.53 (s, 1H), 5.14-4.99 (m, 2H), 3.68-3.51 (m, 8H), 2.95-2.81 (m, 1H), 2.60 (d, J = 2.1 Hz, 2H), 2.31 (s, 3H), 2.06- 1.94 (m, 2H), 1.63-1.44 (m, 8H), 1.37-1.28 (m, 6H) | 805.25 | D | |
| I-137 | 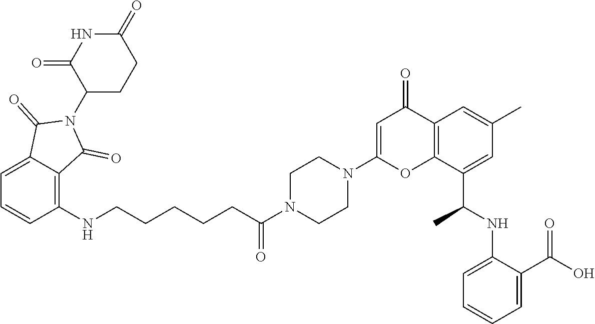 [SMILES: Cc1cc([C@H](C)Nc2ccccc2C(=O)O)c2oc(N3CCN(C(=O)CCCCCNc4cccc5c4C(=O)N(C4CCC(=O)NC4=O)C5=O)CC3)cc(=O)c2c1] | 1H NMR (400 MHz, CD₃SOCD₃, 298K)(ppm) = 12.77 (br s, 1H), 11.09 (s, 1H), 8.38 (br s, 1H), 7.81 (dd, J = 1.5, 7.8 Hz, 1H), 7.63-7.52 (m, 2H), 7.38 (d, J = 1.9 Hz, 1H), 7.28-7.16 (m, 1H), 7.10 (d, J = 8.5 Hz, 1H), 7.01 (d, J = 7.0 Hz, 1H), 6.59-6.50 (m, 2H), 6.46 (d, J = 8.8 Hz, 1H), 5.53 (s, 1H), 5.14-4.98 (m, 2H), 3.65-3.51 (m, 8H), 2.95- 2.81 (m, 1H), 2.59 (br s, 1H), 2.37 (d, J = 7.2 Hz, 2H), 2.30 (s, 3H), 2.06-1.96 (m, 1H), 1.64-1.50 (m, 7H), 1.42-1.32 (m, 2H) | 777.1 | D | |
| I-138 | 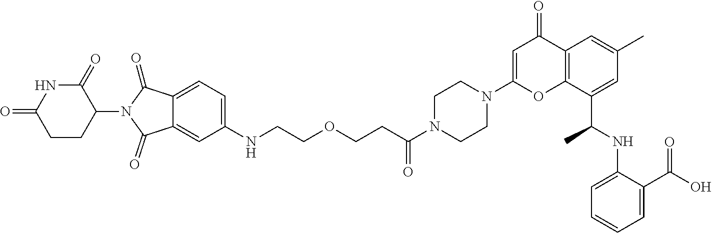 [SMILES: Cc1cc([C@H](C)Nc2ccccc2C(=O)O)c2oc(N3CCN(C(=O)CCOCCNc4ccc5c(c4)C(=O)N(C4CCC(=O)NC4=O)C5=O)CC3)cc(=O)c2c1] | 1H NMR (400 MHz, CD₃SOCD₃, 298K) (ppm) = 12.76 (br s, 1H), 11.05 (s, 1H), 8.36-8.31 (m, 1H), 7.81 (dd, J = 1.7, 8.0 Hz, 1H), 7.60 (d, J = 1.6 Hz, 1H), 7.51 (d, J = 8.4 Hz, 1H), 7.37 (d, J = 1.9 Hz, 1H), 7.28-7.21 (m, 1H), 7.13 (t, J = 5.1 Hz, 1H), 6.99 (d, J = 1.9 Hz, 1H), 6.87 (dd, J = 2.0, 8.4 Hz, 1H), 6.55 (t, J = 7.6 Hz, 1H), 6.46 (d, J = 8.4 Hz, 1H), 5.50 (s, 1H), 5.09 (t, J = 6.3 Hz, 1H), 5.01 (dd, J = 5.4, 12.8 Hz, 1H), 3.70 (t, J = 6.4 Hz, 2H), 3.65-3.52 (m, 10H), 2.92-2.81 (m, 1H), 2.65-2.62 (m, 2H), 2.60-2.55 | 779.3 | D | |
| | | (m, 2H ), 2.30 (s, 3H), 2.04-1.93 | | | |
| | | (m, 2H), 1.58 (d, J = 6.7 Hz, 3H) | | | |
| I-139 | 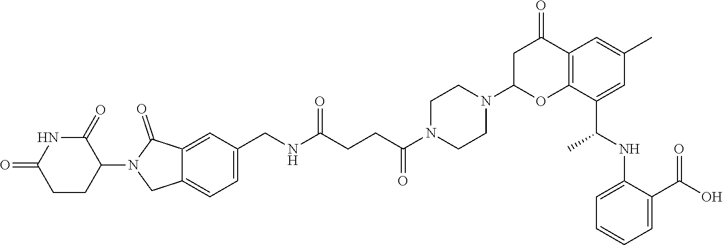 [SMILES: Cc1cc2c(c([C@@H](C)Nc3ccccc3C(=O)O)c1)OC(N1CCN(C(=O)CCC(=O)NCc3ccc4c(c3)C(=O)N(C3CCC(=O)NC3=O)C4)CC1)CC2=O] | | | | |
| I-140 | 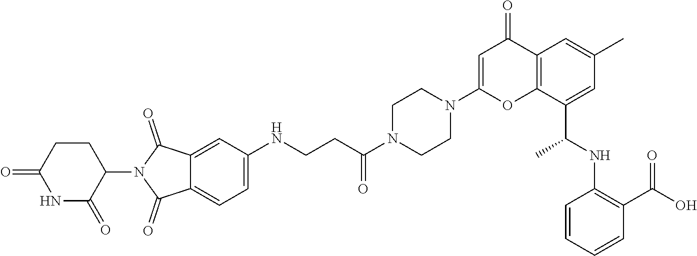 [SMILES: Cc1cc([C@@H](C)Nc2ccccc2C(=O)O)c2oc(N3CCN(C(=O)CCNc4ccc5c(c4)C(=O)N(C4CCC(=O)NC4=O)C5=O)CC3)cc(=O)c2c1] | 1H NMR (400 MHz, DMSO-d6, 25C): = 11.05 (s, 1H), 8.49 (br s, 1H), 7.81 (dd, J = 7.9, 1.6 Hz, 1H), 7.61 (d, J = 1.3 Hz, 1H), 7.57 (d, J = 8.4 Hz, 1H), 7.38 (d, J = 2.1 Hz, 1H), 7.24-7.13 (m, 2H), 6.98 (d, J = 1.9 Hz, 1H), 6.88 (dd, J = 8.4, 2.1 Hz, 1H), 6.53 (t, J = 7.4 Hz, 1H), 6.44 (d, J = 8.4 Hz, 1H), 5.53 (s, 1H), 5.06-5.12 (m, 1H), 5.03 (dd, J = 12.9, 5.4 Hz, 1H), 3.67-3.55 (m, 8H), 3.49-3.45 (m, 2H), 3.01 (td, J = 6.6, 4.0 Hz, 1H), 2.92-2.82 (m, 1H), 2.71-2.68 (m, 1H), 2.62-2.55 (m, 1H), 2.30 (s, 3H), 2.03-1.95 (m, 2H), 1.57 (d, | 735.2 | C | |
| | | J = 6.6 Hz, 3H) | | | |
| I-141 | 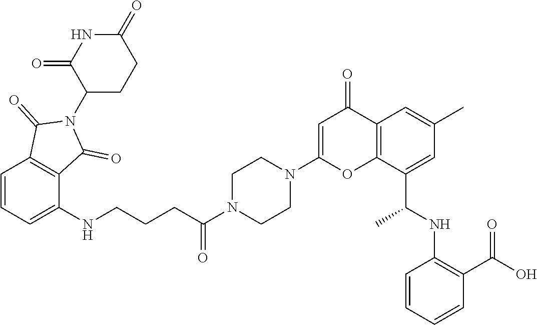 [SMILES: Cc1cc([C@@H](C)Nc2ccccc2C(=O)O)c2oc(N3CCN(C(=O)CCCNc4cccc5c4C(=O)N(C4CCC(=O)NC4=O)C5=O)CC3)cc(=O)c2c1] | 1H NMR (400 MHz, DMSO-d6, 25C): = 12.77 (br s, 1H), 11.09 (s, 1H), 8.35 (br s, 1H), 7.81 (dd, J = 7.9, 1.6 Hz, 1H), 7.61 (s, 1H), 7.56-7.60 (m, 1H), 7.38 (d, J = 2.1 Hz, 1H), 7.28-7.21 (m, 1H), 7.18 (d, J = 8.7 Hz, 1H), 7.02 (d, J = 7.0 Hz, 1H), 6.71-6.65 (m, 1H), 6.55 (t, J = 7.4 Hz, 1H), 6.46 (d, J = 8.4 Hz, 1H), 5.54 (s, 1H), 5.10 (t, J = 6.5 Hz, 1H), 5.07-5.01 (m, 1H), 3.66-3.53 (m, 8H), 3.39- 3.35 (m, 2H), 2.93-2.82 (m, 1H), 2.63-2.59 (m, 1H), 2.44 (br s, 2H), 2.31 (s, 3H), 2.04-1.96 (m, 2H), 1.85-1.79(m, 2H), 1.59 (d, J = 6.6 Hz, 3H) | 749.15 | C | |
| I-142 | 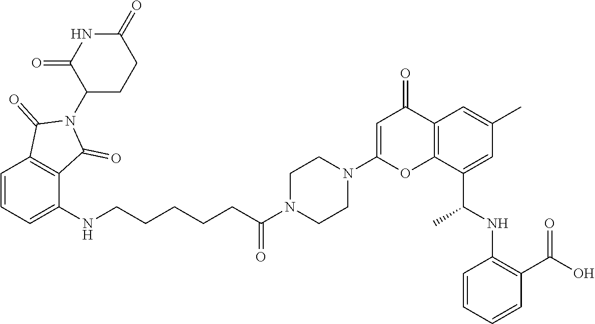 [SMILES: Cc1cc([C@@H](C)Nc2ccccc2C(=O)O)c2oc(N3CCN(C(=O)CCCCCNc4cccc5c4C(=O)N(C4CCC(=O)NC4=O)C5=O)CC3)cc(=O)c2c1] | 1H NMR (400 MHz, CD₃SOCD₃, 298K)(ppm) = 12.77 (br s, 1H), 11.09 (s, 1H), 8.38-8.31 (m, 1H), 7.81 (dd, J = 1.5, 7.9 Hz, 1H), 7.63-7.55 (m, 2H), 7.38 (d, J = 2.1 Hz, 1H), 7.28-7.20 (m, 1H), 7.12- 7.07 (m, 1H), 7.01 (d, J = 7.0 Hz, 1H), 6.59-6.50 (m, 2H), 6.47 (d, J = 8.4 Hz, 1H), 5.53 (s, 1H), 5.15- 5.08 (m, 1H), 5.04 (dd, J = 5.5, 12.8 Hz, 1H), 3.65-3.51 (m, 8H), 2.92-2.82 (m, 1H), 2.60 (br s, 2H), 2.36 (t,J = 7.4 Hz, 2H), 2.31 (s, 3H), 2.06-1.98 (m, 1H), 1.63-1.50 (m, 7H), 1.42-1.33 (m, 2H) | 777.1 | C | |
| I-143 | 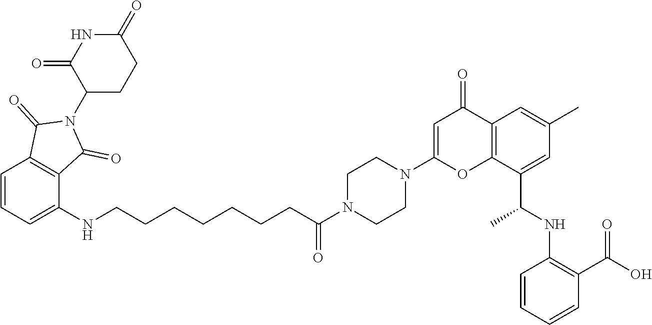 [SMILES: Cc1cc([C@@H](C)Nc2ccccc2C(=O)O)c2oc(N3CCN(C(=O)CCCCCCCNc4cccc5c4C(=O)N(C4CCC(=O)NC4=O)C5=O)CC3)cc(=O)c2c1] | 1H NMR (400 MHz, CD₃SOCD₃, 298K)(ppm) = 12.76 (br s, 1H), 11.09 (s, 1H), 8.35 (br s, 1H), 7.81 (dd, J = 1.5, 7.8 Hz, 1H), 7.63-7.53 (m, 2H), 7.38 (d, J = 2.1 Hz, 1H), 7.28-7.20 (m, 1H), 7.09 (d, J = 8.7 Hz, 1H), 7.01 (d, J = 7.0 Hz, 1H), 6.58-6.50 (m, 2H), 6.47 (d, J = 8.7 Hz, 1H), 5.53 (s, 1H), 5.07 (s, 2H), 3.65-3.52 (m, 8H), 2.94-2.81 (m, 1H), 2.60 (d, J = 2.8 Hz, 2H), 2.31 (s, 3H), 2.07-1.95 (m, 3H), 1.63- 1.43 (m, 8H), 1.36-1.30 (m, 6H) | 805.3 | D | |
| I-144 | 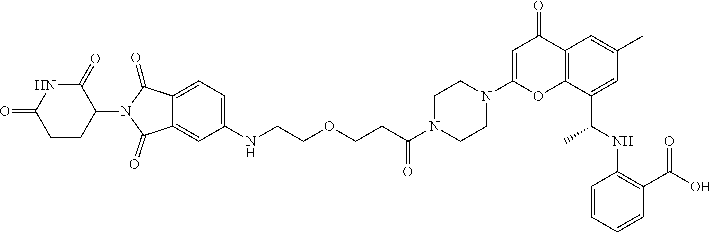 [SMILES: Cc1cc([C@@H](C)Nc2ccccc2C(=O)O)c2oc(N3CCN(C(=O)CCOCCNc4ccc5c(c4)C(=O)N(C4CCC(=O)NC4=O)C5=O)CC3)cc(=O)c2c1] | 1H NMR (400 MHz, CD₃SOCD₃, 298K)(ppm) = 12.76 (br s, 1H), 11.05 (s, 1H), 8.34 (d, J = 5.9 Hz, 1H), 7.81 (dd, J = 1.6, 7.9 Hz, 1H), 7.60 (d, J = 1.5 Hz, 1H), 7.51 (d, J = 8.4 Hz, 1H), 7.37 (d, J = 2.1 Hz, 1H), 7.27-7.21 (m, 1H), 7.13 (t, J = 5.5 Hz, 1H), 6.99 (d, J = 1.9 Hz, 1H), 6.87 (dd, J = 2.1, 8.5 Hz, 1H), 6.55 (t, J = 7.6 Hz, 1H), 6.46 (d, J = 8.4 Hz, 1H), 5.50 (s, 1H), 5.09 (t, J = 6.4 Hz, 1H), 5.01 (dd, J = 5.4, 13.0 Hz, 1H), 3.70 (t, J = 6.4 Hz, 2H), 3.64-3.51 (m, 10H), | 779.15 | D | |
| | | 3.06-2.98 (m, 1H), 2.92-2.81 (m, | | | |
| | | 1H), 2.65-2.62 (m, 2H), 2.60-2.55 | | | |
| | | (m, 2H ), 2.30 (s, 3H), 2.05-1.90 | | | |
| | | (m, 2H), 1.58 (d, J = 6.6 Hz, 3H) | | | |
| I-145 | 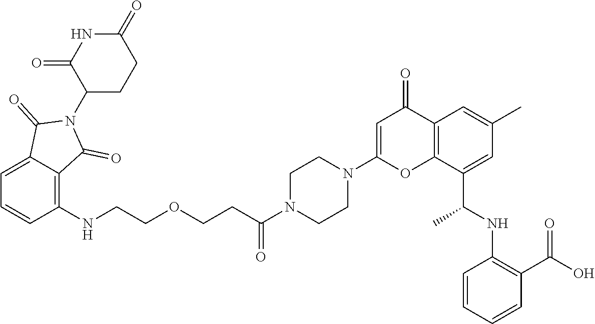 [SMILES: Cc1cc([C@@H](C)Nc2ccccc2C(=O)O)c2oc(N3CCN(C(=O)CCOCCNc4cccc5c4C(=O)N(C4CCC(=O)NC4=O)C5=O)CC3)cc(=O)c2c1] | 1H NMR (400 MHz, DMSO-d6) = 12.77 (br s, 1H), 11.09 (s, 1H), 8.34 (br d, J = 6.5 Hz, 1H), 7.81 (dd, J = 1.5, 7.9 Hz, 1H), 7.62-7.54 (m, 2H), 7.37 (d, J = 1.9 Hz, 1H), 7.27-7.20 (m, 1H), 7.13 (d, J = 8.5 Hz, 1H), 7.02 (d, J = 6.9 Hz, 1H), 6.61-6.52 (m, 2H), 6.46 (d, J = 8.5 Hz, 1H), 5.52 (s, 1H), 5.09 (br t, J = 6.5 Hz, 1H), 5.07-5.00 (m, 1H), 3.70 (t, J = 6.5 Hz, 2H), 3.65-3.51 (m, 10H), 3.49-3.43 (m, 2H), 2.93-2.79 (m, 1H), 2.63 (br t, J = 6.5 Hz, 2H), 2.58 (br s, 1H), 2.30 (s, 3H), 2.05- 1.94 (m, 2H), 1.58 (d, J = 6.5 Hz, 3H) | 779.1 | D | |
| I-146 | 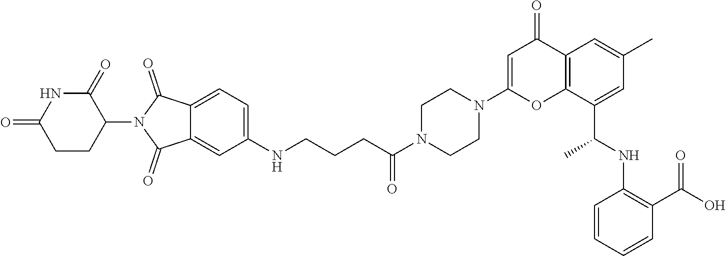 [SMILES: Cc1cc([C@@H](C)Nc2ccccc2C(=O)O)c2oc(N3CCN(C(=O)CCCNc4ccc5c(c4)C(=O)N(C4CCC(=O)NC4=O)C5=O)CC3)cc(=O)c2c1] | 1H NMR (400 MHz, CD₃SOCD₃, 298K)(ppm) = 12.76 (br s, 1H), 11.05 (s, 1H), 8.35 (d, J = 6.3 Hz, 1H), 7.81 (dd, J = 1.7, 8.0 Hz, 1H), 7.64-7.53 (m, 2H), 7.38 (d, J = 2.1 Hz, 1H), 7.25 (dt, J = 1.6, 7.8 Hz, 1H), 7.21-7.14 (m, 1H), 6.96 (s, 1H), 6.86 (dd, J = 2.0, 8.4 Hz, 1H), 6.56 (t, J = 7.6 Hz, 1H), 6.47 (d, J = 8.4 Hz, 1H), 5.54 (s, 1H), 5.11 (t, J = 6.4 Hz, 1H), 5.02 (dd, J = 5.4, 12.8 Hz, 1H), 3.66-3.56 (m, 8H), 3.21 (d, J = 6.6 Hz, 2H), 2.91- 2.81 (m, 1H), 2.57 (br s, 2H), 2.31 (s, 3H), 2.04-1.95 (m, 3H), 1.86- 1.79 (m, 2H), 1.59 (d, J = 6.6 Hz, | 749.5 | D | |
| | | 3H) | | | |
| I-147 | 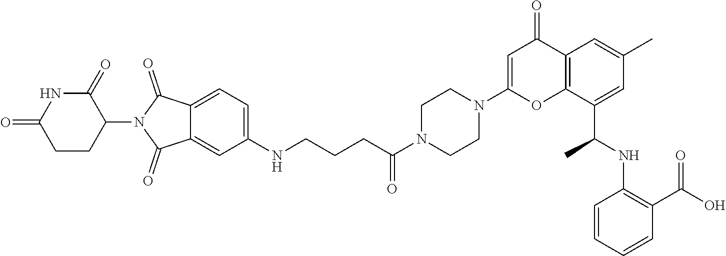 [SMILES: Cc1cc([C@H](C)Nc2ccccc2C(=O)O)c2oc(N3CCN(C(=O)CCCNc4ccc5c(c4)C(=O)N(C4CCC(=O)NC4=O)C5=O)CC3)cc(=O)c2c1] | 1H NMR (400 MHz, CD₃SOCD₃, 298K)(ppm) = 12.76 (br s, 1H), 11.05 (s, 1H), 8.35 (br s, 1H), 7.84- 7.77 (m, 1H), 7.61 (s, 1H), 7.56 (d, J = 8.4 Hz, 1H), 7.38 (s, 1H), 7.29- 7.15 (m, 2H), 6.96 (s, 1H), 6.89- 6.83 (m, 1H), 6.55 (t, J = 7.4 Hz, 1H), 6.46 (d, J = 8.4 Hz, 1H), 5.54 (s, 1H), 5.10 (t, J = 6.4 Hz, 1H), 5.02 (dd, J = 5.3, 12.9 Hz, 1H), 3.67-3.55 (m, 8H), 3.21 (d, J = 6.3 Hz, 2H), 2.93-2.79 (m, 1H), 2.30 (s, 3H), 2.04-1.94 (m, 3H), 1.82 (t, J = 7.0 Hz, 2H), 1.58 (d, J = 6.5 Hz, 3H) | 749.5 | D | |
| I-148 | 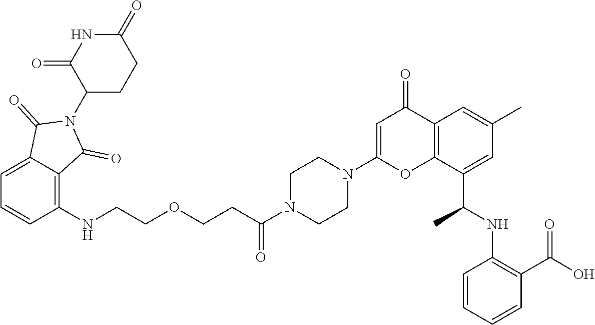 [SMILES: Cc1cc([C@H](C)Nc2ccccc2C(=O)O)c2oc(N3CCN(C(=O)CCOCCNc4cccc5c4C(=O)N(C4CCC(=O)NC4=O)C5=O)CC3)cc(=O)c2c1] | 1H NMR (400 MHz, DMSO-d6) = 12.76 (br s, 1H), 11.09 (s, 1H), 8.35 (d, J = 5.7 Hz, 1H), 7.81 (dd, J = 1.4, 8.0 Hz, 1H), 7.63-7.53 (m, 2H), 7.37 (d, J = 1.8 Hz, 1H), 7.27-7.20 (m, 1H), 7.13 (d, J = 8.7 Hz, 1H), 7.02 (d, J = 7.0 Hz, 1H), 6.61-6.52 (m, 2H), 6.46 (d, J = 8.5 Hz, 1H), 5.52 (s, 1H), 5.09 (t, J = 6.5 Hz, 1H), 5.04 (dd, J = 5.2, 13.7 Hz, 1H), 3.70 (t, J = 6.5 Hz, 2H), 3.61 (d, J = 5.0 Hz, 8H), 3.54 (d, J = 3.1 Hz, 2H), 3.49-3.45 (m, 2H), 2.92-2.80 (m, 1H), 2.65-2.62 (m, 2H), 2.58 (d, J = 2.1 Hz, 1H), 2.30 (s, 3H), 2.04-1.95 (m, 2H), 1.58 (d, J = 6.6 Hz, 3H) | 779.1 | D | |
| I-149 | 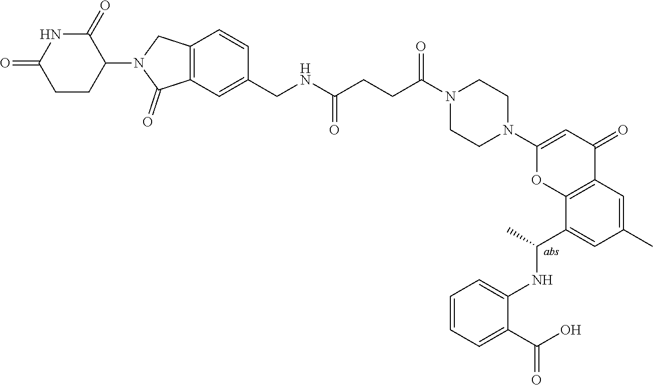 [SMILES: Cc1cc([C@@H](C)Nc2ccccc2C(=O)O)c2oc(N3CCN(C(=O)CCC(=O)NCc4ccc5c(c4)C(=O)N(C4CCC(=O)NC4=O)C5)CC3)cc(=O)c2c1] | 1H NMR (400 MHz, CD₃SOCD₃, 298K)(ppm) = 12.78 (br s, 1H), 10.98 (s, 1H), 8.46 (t, J = 6.2 Hz, 1H), 8.38-8.33 (m, 1H), 7.81 (dd, J = 1.6, 7.9 Hz, 1H), 7.66-7.60 (m, 2H), 7.53 (d, J = 1.5 Hz, 2H), 7.38 (d, J = 2.1 Hz, 1H), 7.29-7.17 (m, 1H), 6.55 (t, J = 7.5 Hz, 1H), 6.47 (d, J = 8.5 Hz, 1H), 5.53 (s, 1H), 5.13-5.06 (m, 2H), 4.45-4.25 (m, 4H), 3.63 (br s, 6H), 3.55 (d, J = 2.8 Hz, 2H), 2.95-2.84 (m, 1H), 2.62 (d, J = 6.9 Hz, 2H), 2.58-2.56 (m, 1H), 2.42-2.35 (m, 1H), 2.31 (s, 3H), 2.03-1.94 (m, 2H), 1.59 (d, J = 6.6 Hz, 3H) | 763.3 | D | |
| I-150 | 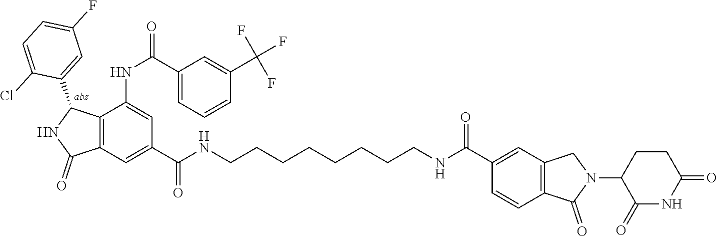 [SMILES: O=C1CCC(N2Cc3cc(C(=O)NCCCCCCCCNC(=O)c4cc(NC(=O)c5cccc(C(F)(F)F)c5)c5c(c4)C(=O)N[C@@H]5c4cc(F)ccc4Cl)ccc3C2=O)C(=O)N1] | 1H NMR (400 MHz, DMSO-d6) ppm 11.00 (br s, 1 H) 10.55 (s, 1 H) 9.23 (d, J = 0.73 Hz, 1 H) 8.78 (t, J = 5.36 Hz, 1 H) 8.62 (t, J = 5.58 Hz, 1 H) 8.20 (s, 1 H) 8.04 (s, 1 H) 7.89-8.01 (m, 4 H) 7.76-7.83 (m, 2 H) 7.67-7.74 (m, 1 H) 7.31 (dd, J = 8.80, 5.14 Hz, 1 H) 7.09 (br dd, J = 8.22, 2.79 Hz, 1 H) 5.97-6.13 (m, 1 H) 5.13 (dd, J = 13.20, 5.14 Hz, 1 H) 4.28-4.56 (m, 2 H) 3.22-3.30 (m, 4 H) 2.86- 2.97 (m, 1 H) 2.58-2.64 (m, 1 H) 2.42 (br dd, J = 12.91, 4.40 Hz, 1 H) 1.93-2.06 (m, 2 H) 1.55 (d, | 888.75 | D | |
| | | J = 5.28 Hz, 4 H) 1.33 (br s, 8 H) | | | |
| I-151 | 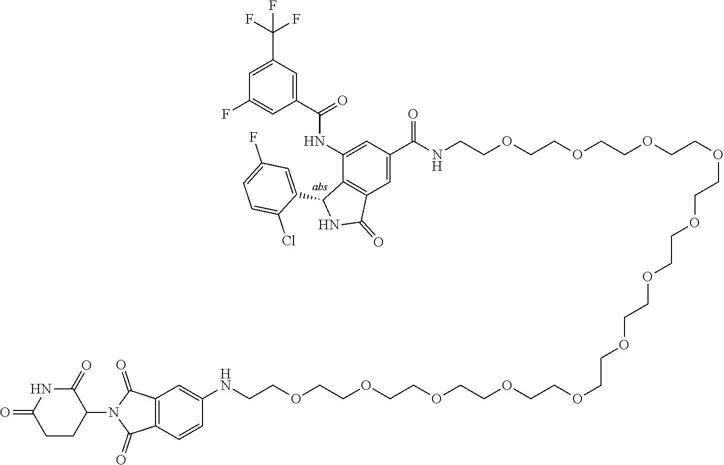 [SMILES: O=C1CCC(N2C(=O)c3ccc(NCCOCCOCCOCCOCCOCCOCCOCCOCCOCCOCCOCCOCCNC(=O)c4cc(NC(=O)c5cc(F)cc(C(F)(F)F)c5)c5c(c4)C(=O)N[C@@H]5c4cc(F)ccc4Cl)cc3C2=O)C(=O)N1] | 1H NMR (400 MHz, CHLORO- FORM-d) ppm 9.02-9.14 (m, 1 H) 8.32-8.46 (m, 2 H) 8.23 (s, 1 H) 7.77 (d, J = 2.79 Hz, 1 H) 7.60- 7.68 (m, 2 H) 7.57 (d, J = 8.36 Hz, 1 H) 7.47 (d, J = 8.22 Hz, 1 H) 7.20-7.25 (m, 1 H) 6.96 (t, J = 1.76 Hz, 1 H) 6.86-6.95 (m, 1 H) 6.74-6.81 (m, 2 H) 6.56-6.66 (m, 1 H) 6.29 (br s, 1 H) 5.60 (t, J = 5.14 Hz, 1 H) 4.91 (dd, J = 12.25, 5.35 Hz, 1 H) 3.69-3.75 (m, 5 H) 3.48-3.66 (m, 46 H) 2.70-2.91 (m, 3 H) 1.98-2.24 (m, 2 H) | 1337.05 | D | |
| I-152 | 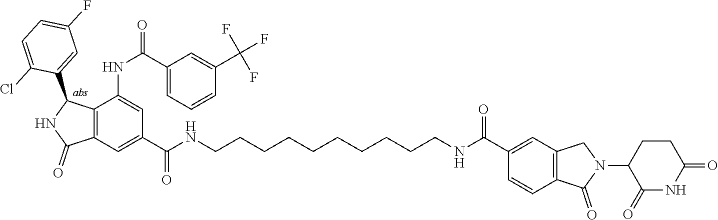 [SMILES: O=C1CCC(N2Cc3cc(C(=O)NCCCCCCCCCCNC(=O)c4cc(NC(=O)c5cccc(C(F)(F)F)c5)c5c(c4)C(=O)N[C@H]5c4cc(F)ccc4Cl)ccc3C2=O)C(=O)N1] | 1H NMR (400 MHz, DMSO-d6) ppm 11.00 (s, 1 H) 10.55 (s, 1 H) 9.23 (br s, 1 H) 8.77 (t, J = 5.28 Hz, 1 H) 8.61 (t, J = 5.36 Hz, 1 H) 8.20-8.26 (m, 1 H) 8.04 (s, 1 H) 7.91-7.99 (m, 4 H) 7.77-7.82 (m, 2 H) 7.68-7.74 (m, 1 H) 7.31 (dd, J = 8.80, 5.14 Hz, 1 H) 7.08 (td, J = 8.36, 3.08 Hz, 1 H) 5.98-6.11 (m, 1 H) 5.14 (dd, J = 13.20, 4.99 Hz, 1 H) 4.34-4.57 (m, 2 H) 3.24- 3.29 (m, 4 H) 2.85-2.97 (m, 1 H) 2.62 (br s, 1 H) 2.38-2.43 (m, 1 H) 1.95-2.05 (m, 2 H) 1.54 (d, J = 5.87 | 917.2 | B | |
| | | Hz, 4 H) 1.29 (br s, 12 H) | | | |
| I-153 | 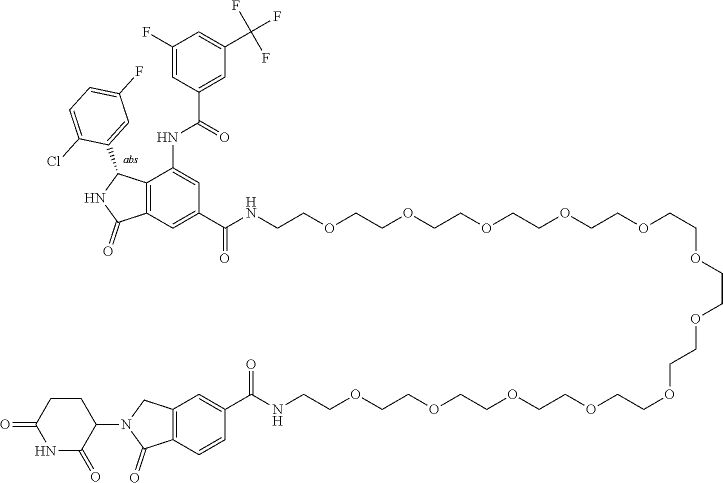 [SMILES: O=C1CCC(N2Cc3cc(C(=O)NCCOCCOCCOCCOCCOCCOCCOCCOCCOCCOCCOCCOCCNC(=O)c4cc(NC(=O)c5cc(F)cc(C(F)(F)F)c5)c5c(c4)C(=O)N[C@@H]5c4cc(F)ccc4Cl)ccc3C2=O)C(=O)N1] | 1H NMR (400 MHz, DMSO-d6) ppm 11.00 (br s, 1 H) 10.63 (br s, 1 H) 9.25 (br s, 1 H) 8.86 (br s, 1 H) 8.70 (t, J = 5.43 Hz, 1 H) 8.23 (s, 1 H) 8.05 (s, 1 H) 7.92- 8.01 (m, 3 H) 7.80 (d, J = 7.92 Hz, 1 H) 7.75 (d, J = 8.80 Hz, 1 H) 7.68 (s, 1 H) 7.32 (dd, J = 8.80, 5.14 Hz, 1 H) 7.09 (td, J = 8.36, 2.93 Hz, 1 H) 5.95-6.09 (m, 1 H) 5.14 (dd, J = 13.28, 5.06 Hz, 1 H) 4.48-4.55 (m, 1 H) 4.32-4.44 (m, 1 H) 3.52-3.58 (m, 12 H) 3.46- 3.51 (m, 41 H) 2.85-2.96 (m, 1 H) 2.62 (d, J = 2.49 Hz, 1 H) 2.36-2.44 (m, 1 H) 1.97-2.08 (m, 1 H) | 1353.25 | D | |
| I-154 | 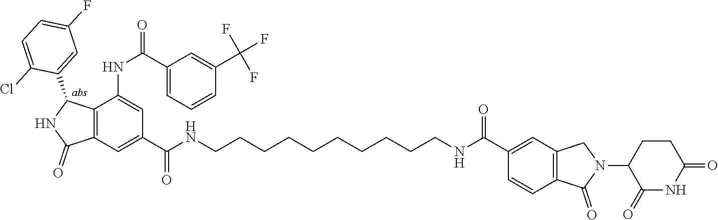 [SMILES: O=C1CCC(N2Cc3cc(C(=O)NCCCCCCCCCCNC(=O)c4cc(NC(=O)c5cccc(C(F)(F)F)c5)c5c(c4)C(=O)N[C@@H]5c4cc(F)ccc4Cl)ccc3C2=O)C(=O)N1] | 1H NMR (400 MHz, DMSO-d6) ppm 11.00 (s, 1 H) 10.55 (s, 1 H) 9.23 (d, J = 1.03 Hz, 1 H) 8.77 (t, J = 5.43 Hz, 1 H) 8.62 (t, J = 5.58 Hz, 1 H) 8.20 (s, 1 H) 8.04 (s, 1 H) 7.90-8.00 (m, 4 H) 7.76-7.82 (m, 2 H) 7.68-7.74 (m, 1 H) 7.31 (dd, J = 8.80, 5.14 Hz, 1 H) 7.02-7.12 (m, 1 H) 5.98-6.10 (m, 1 H) 5.14 (dd, J = 13.35, 5.14 Hz, 1 H) 4.33- 4.54 (m, 2 H) 3.24-3.30 (m, 4 H) 2.84-2.97 (m, 1 H) 2.62 (br s, 1 H) 2.35-2.45 (m, 1 H) 1.97-2.07 (m, 1 H) 1.54 (d, J = 6.31 Hz, 4 H) | 917.15 | D | |
| | | 1.29 (br s, 12 H) | | | |
| I-155 | 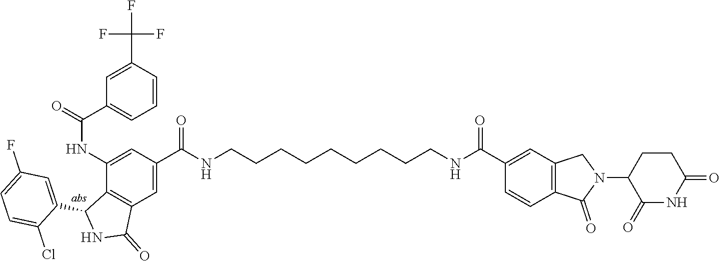 [SMILES: O=C1CCC(N2Cc3cc(C(=O)NCCCCCCCCCNC(=O)c4cc(NC(=O)c5cccc(C(F)(F)F)c5)c5c(c4)C(=O)N[C@@H]5c4cc(F)ccc4Cl)ccc3C2=O)C(=O)N1] | 1H NMR (400 MHz, DMSO-d6) ppm 11.00 (br s, 1 H) 10.57 (br s, 1 H) 9.23 (br s, 1 H) 8.77 (t, J = 5.50 Hz, 1 H) 8.62 (t, J = 5.43 Hz, 1 H) 8.20 (s, 1 H) 8.04 (s, 1 H) 7.88-8.01 (m, 4 H) 7.77-7.85 (m, 2 H) 7.66-7.73 (m, 1 H) 7.31 (dd, J = 8.88, 5.21 Hz, 1 H) 7.08 (td, J = 8.36, 3.08 Hz, 1 H) 5.99-6.12 (m, 1 H) 5.13 (dd, J = 13.20, 5.14 Hz, 1 H) 4.32-4.57 (m, 2 H) 3.23- 3.30 (m, 4 H) 2.85-2.98 (m, 1 H) 2.57-2.62 (m, 1 H) 2.35-2.43 (m, 1 H) 1.95-2.07 (m, 2 H) 1.54 (d, J = 5.28 Hz, 4 H) 1.31 (br s, 10 H) | 903.05 | D | |
| I-156 | 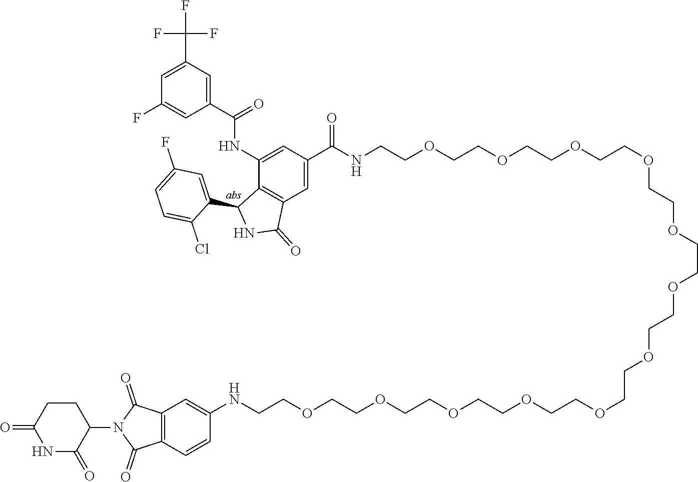 [SMILES: O=C1CCC(N2C(=O)c3ccc(NCCOCCOCCOCCOCCOCCOCCOCCOCCOCCOCCOCCOCCNC(=O)c4cc(NC(=O)c5cc(F)cc(C(F)(F)F)c5)c5c(c4)C(=O)N[C@H]5c4cc(F)ccc4Cl)cc3C2=O)C(=O)N1] | 1H NMR (400 MHz, DMSO-d6) ppm 11.05 (s, 1 H) 10.62 (s, 1 H) 9.25 (d, J = 1.47 Hz, 1 H) 8.76- 8.91 (m, 1 H) 8.23 (s, 1 H) 7.91- 8.04 (m, 2 H) 7.75 (d, J = 9.68 Hz, 1 H) 7.68 (s, 1 H) 7.55 (d, J = 8.36 Hz, 1 H) 7.32 (dd, J = 8.80, 5.28 Hz, 1 H) 7.15 (t, J = 5.65 Hz, 1 H) 7.09 (d, J = 3.37 Hz, 1 H) 7.00 (d, J = 2.05 Hz, 1 H) 6.89 (dd, J = 8.44, 1.98 Hz, 1 H) 4.98-5.07 (m, 1 H) 3.41-3.64 (m, 48 H) 2.80-2.90 (m, 1 H) 2.58 (br s, 2 H) 1.96-2.01 (m, 3 H) | 1335.25 | A | |
| I-157 | 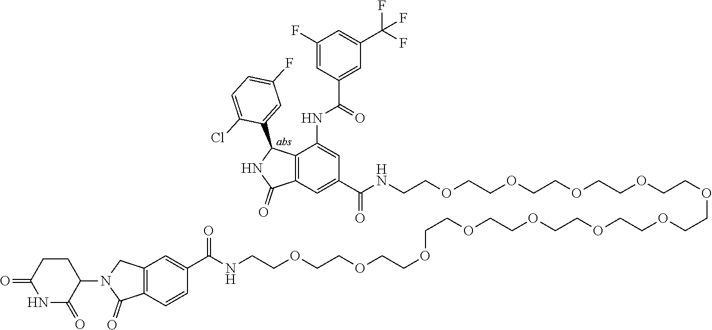 [SMILES: O=C1CCC(N2Cc3cc(C(=O)NCCOCCOCCOCCOCCOCCOCCOCCOCCOCCOCCOCCOCCNC(=O)c4cc(NC(=O)c5cc(F)cc(C(F)(F)F)c5)c5c(c4)C(=O)N[C@H]5c4cc(F)ccc4Cl)ccc3C2=O)C(=O)N1] | 1H NMR (400 MHz, DMSO-d6) ppm 11.00 (s, 1 H) 10.62 (s, 1 H) 9.25 (br s, 1 H) 8.82-8.91 (m, 1 H) 8.70 (t, J = 5.43 Hz, 1 H) 8.23 (s, 1 H) 8.05 (s, 1 H) 7.93-8.01 (m, 3 H) 7.81 (s, 1 H) 7.75 (d, J = 8.66 Hz, 1 H) 7.68 (s, 1 H) 7.32 (dd, J = 8.95, 5.14 Hz, 1 H) 7.09 (td, J = 8.25, 2.57 Hz, 1 H) 5.87-6.06 (m, 1 H) 5.10-5.18 (m, 1 H) 4.48- 4.55 (m, 1 H) 4.41 (s, 1 H) 3.52- 3.58 (m, 12 H) 3.45-3.52 (m, 41 H) 2.86-2.97 (m, 1 H) 2.59- 2.65 (m, 1 H) 2.38-2.45 (m, 1 H) 1.96-2.07 (m, 1 H) | 1352.6 | C | |
| I-158 | 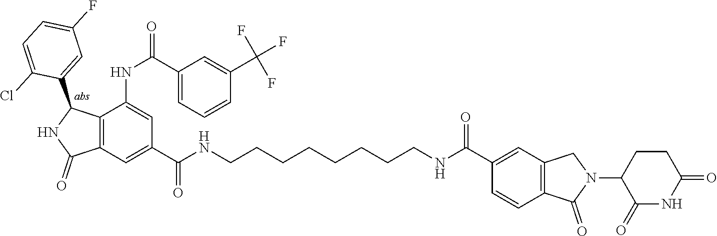 [SMILES: O=C1CCC(N2Cc3cc(C(=O)NCCCCCCCCNC(=O)c4cc(NC(=O)c5cccc(C(F)(F)F)c5)c5c(c4)C(=O)N[C@H]5c4cc(F)ccc4Cl)ccc3C2=O)C(=O)N1] | 1H NMR (400 MHz, DMSO-d6) ppm 11.00 (br s, 1 H) 10.55 (s, 1 H) 9.23 (br s, 1 H) 8.78 (t, J = 5.58 Hz, 1 H) 8.62 (t, J = 5.58 Hz, 1 H) 8.20 (s, 1 H) 8.04 (s, 1 H) 7.90-8.00 (m, 4 H) 7.77-7.82 (m, 2 H) 7.67-7.73 (m, 1 H) 7.31 (dd, J = 8.80, 5.14 Hz, 1 H) 7.08 (td, J = 8.36, 3.08 Hz, 1 H) 5.91-6.13 (m, 1 H) 5.13 (dd, J = 13.28, 5.06 Hz, 1 H) 4.26-4.58 (m, 2 H) 3.17- 3.30 (m, 4 H) 2.83-2.97 (m, 1 H) 2.59-2.64 (m, 1 H) 2.42 (dd, J = 13.13, 4.47 Hz, 1 H) 1.97-2.05 (m, 1 H) 1.55 (d, J = 4.11 Hz, 4 H) | 888.8 | A | |
| | | 1.33 (br s, 8 H) | | | |
| I-159 | 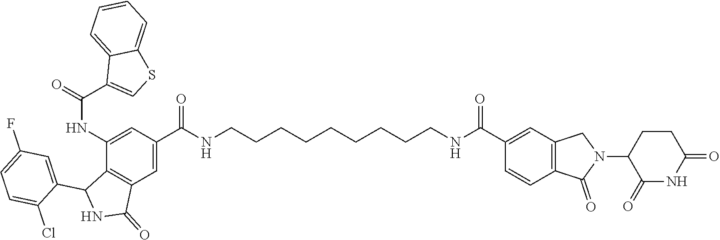 [SMILES: O=C1CCC(N2Cc3cc(C(=O)NCCCCCCCCCNC(=O)c4cc(NC(=O)c5csc6ccccc56)c5c(c4)C(=O)NC5c4cc(F)ccc4Cl)ccc3C2=O)C(=O)N1] | 1H NMR (400 MHz, CD₃SOCD₃, 298K)(ppm) = 11.00 (br s, 1H), 10.36 (s, 1H), 9.23 (br s, 1H), 8.77 t, J = 5.5 Hz, 1H), 8.62 (t, J = 5.4 Hz, 1H), 8.25-8.15 (m, 2H), 8.08- 7.99 (m, 4H), 7.95 (d, J = 7.8 Hz, 1H), 7.79 (d, J = 7.9 Hz, 1H), 7.46- 7.39 (m, 2H), 7.33 (dd, J = 5.2, 8.9 Hz, 1H), 7.15-7.04 (m, 1H), 6.17 (d, J = 6.5 Hz, 1H), 5.13 (dd, J = 5.2, 13.3 Hz, 1H), 4.58-4.32 (m, 2H), 3.27 (d, J = 6.7 Hz, 4H), 2.94-2.85 (m, 1H), 2.65-2.57 (m, 2H), 2.43-2.38 (m, 1H), 2.05-1.95 (m, 1H), 1.54 (d, J = 6.7 Hz, 4H), | 890.45 | A | |
| | | 1.31 (br s, 10H) | | | |
| I-160 | 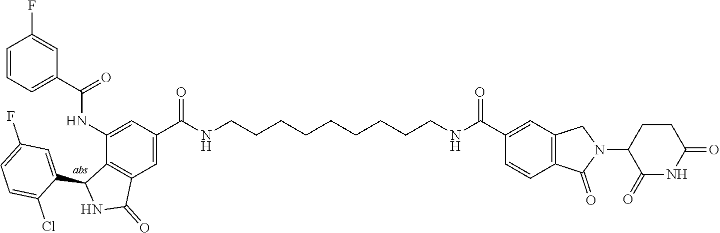 [SMILES: O=C1CCC(N2Cc3cc(C(=O)NCCCCCCCCCNC(=O)c4cc(NC(=O)c5cccc(F)c5)c5c(c4)C(=O)N[C@H]5c4cc(F)ccc4Cl)ccc3C2=O)C(=O)N1] | 1H NMR (400 MHz, DMSO-d6) ppm 11.00 (s, 1 H) 10.35 (s, 1 H) 9.23 (br s, 1 H) 8.76 (t, J = 5.43 Hz, 1 H) 8.62 (t, J = 5.50 Hz, 1 H) 8.19 (s, 1 H) 8.04 (s, 1 H) 7.99 (d, J = 0.88 Hz, 1 H) 7.95 (d, J = 7.92 Hz, 1 H) 7.80 (d, J = 7.92 Hz, 1 H) 7.31-7.53 (m, 5 H) 7.06-7.15 (m, 1 H) 6.00-6.12 (m, 1 H) 5.14 (dd, J = 13.20, 5.14 Hz, 1 H) 4.48-4.54 (m, 1 H) 4.33-4.42 (m, 1 H) 3.23- 3.31 (m, 4 H) 2.85-2.97 (m, 1 H) 2.60 (d, J = 18.19 Hz, 2 H) 2.42 (br dd, J = 13.20, 4.40 Hz, 1 H) | 852.85 | B | |
| | | 1.97-2.06 (m, 1 H) 1.54 (d, J = 3.96 | | | |
| | | Hz, 4 H) 1.26-1.39 (m, 10 H) | | | |
| I-161 | 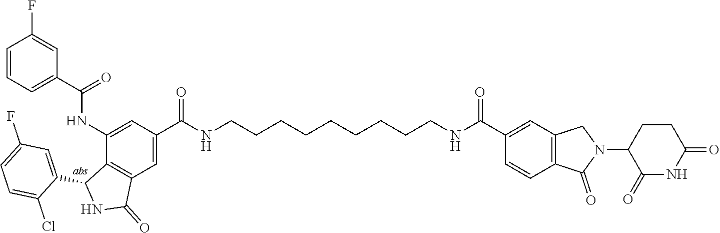 [SMILES: O=C1CCC(N2Cc3cc(C(=O)NCCCCCCCCCNC(=O)c4cc(NC(=O)c5cccc(F)c5)c5c(c4)C(=O)N[C@@H]5c4cc(F)ccc4Cl)ccc3C2=O)C(=O)N1] | 1H NMR (400 MHz, DMSO-d6) ppm 11.00 (s, 1 H) 10.35 (s, 1 H) 9.22 (br s, 1 H) 8.76 (t, J = 5.36 Hz, 1 H) 8.62 (t, J = 5.50 Hz, 1 H) 8.19 (s, 1 H) 7.92-8.07 (m, 3 H) 7.80 (d, J = 7.92 Hz, 1 H) 7.31- 7.54 (m, 5 H) 7.05-7.18 (m, 1 H) 5.99-6.11 (m, 1 H) 5.14 (dd, J = 13.28, 5.06 Hz, 1 H) 4.47-4.57 (m, 1 H) 4.34-4.44 (m, 1 H) 3.22-3.30 (m, 4 H) 2.85-2.94 (m, 1 H) 2.60 (d, J = 18.19 Hz, 2 H) 2.36-2.46 (m, 1 H) 1.94-2.06 (m, 1 H) 1.54 (d, J = 3.96 Hz, 4 H) 1.26-1.39 (m, 10 H) | 852.85 | D | |
| I-162 | 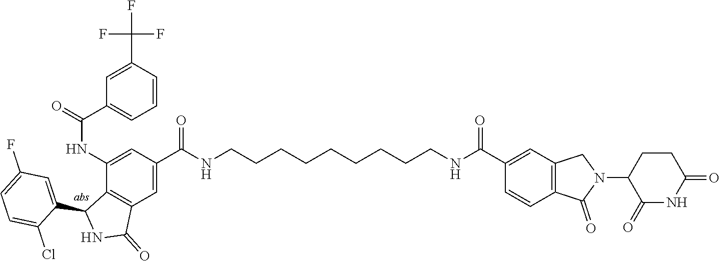 [SMILES: O=C1CCC(N2Cc3cc(C(=O)NCCCCCCCCCNC(=O)c4cc(NC(=O)c5cccc(C(F)(F)F)c5)c5c(c4)C(=O)N[C@H]5c4cc(F)ccc4Cl)ccc3C2=O)C(=O)N1] | 1H NMR (400 MHz, DMSO-d6) ppm 11.00 (br s, 1 H) 10.62 (br s, 1 H) 9.24 (br s, 1 H) 8.78 (br s, 1 H) 8.63 (t, J = 5.43 Hz, 1 H) 8.19 (br s, 1 H) 7.87-8.10 (m, 5 H) 7.75- 7.84 (m, 2 H) 7.70 (t, J = 7.56 Hz, 1 H) 7.31 (dd, J = 8.95, 5.14 Hz, 1 H) 7.02-7.15 (m, 1 H) 5.92-6.18 (m, 1 H) 5.13 (dd, J = 13.28, 5.06 Hz, 1 H) 4.46-4.60 (m, 1 H) 4.31- 4.43 (m, 1 H) 3.14-3.27 (m, 4 H) 2.86-2.97 (m, 1 H) 2.62 (d, J = 2.49 Hz, 1 H) 2.39-2.44 (m, 1 H) 1.95-2.05 (m, 2 H) 1.48-1.59 (m, 4 H) 1.27-1.39 (m, 10 H) | 903.15 | C | |

In chemical structures in Table 1, above, and the Examples, below, stereogenic centers are described according to the Enhanced Stereo Representation format (MDL/Biovia, e.g. using labels “or1”, “or2”, “abs”, “and1”).

In some embodiments, the present disclosure provides a compound in Table 1, above, wherein the compound is denoted as having an ADP-Glo IC₅₀ of “A”. In some embodiments, the present disclosure provides a compound in Table 1, above, wherein the compound is denoted as having an ADP-Glo IC₅₀ of “A” or “B”. In some embodiments, the present disclosure provides a compound in Table 1, above, wherein the compound is denoted as having an ADP-Glo IC₅₀ of “A” or “B” or “C”. In some embodiments, the present disclosure provides a compound in Table 1, above, wherein the compound is denoted as having an ADP-Glo IC₅₀ of “A” or “B” or “C” or “D”.

In some embodiments, the present disclosure provides a compound in Table 1, above, wherein the compound is denoted as having an MCF10A IC₅₀ of “A”. In some embodiments, the present disclosure provides a compound in Table 1, above, wherein the compound is denoted as having an MCF10A IC₅₀ of “A” or “B”. In some embodiments, the present disclosure provides a compound in Table 1, above, wherein the compound is denoted as having an MCF10A IC₅₀ of “A” or “B” or “C”. In some embodiments, the present disclosure provides a compound in Table 1, above, wherein the compound is denoted as having an MCF10A IC₅₀ of “A” or “B” or “C” or “D”.

In some embodiments, the present disclosure comprises a compound of formula I selected from those depicted in Table 1, above, or a pharmaceutically acceptable salt, stereoisomer, or mixture of stereoisomers thereof. In some embodiments, the present disclosure provides a compound of formula I selected from those depicted in Table 1, above, or a pharmaceutically acceptable salt thereof. In some embodiments, the present disclosure provides a compound of formula I selected from those depicted in Table 1, above.

In some embodiments, the present disclosure comprises a compound of formula I-a selected from those depicted in Table 1, above, or a pharmaceutically acceptable salt, stereoisomer, or mixture of stereoisomers thereof. In some embodiments, the present disclosure provides a compound of formula I-a selected from those depicted in Table 1, above, or a pharmaceutically acceptable salt thereof. In some embodiments, the present disclosure provides a compound of formula I-a selected from those depicted in Table 1, above.

In some embodiments, the present disclosure comprises a compound of formula I-b selected from those depicted in Table 1, above, or a pharmaceutically acceptable salt, stereoisomer, or mixture of stereoisomers thereof. In some embodiments, the present disclosure provides a compound of formula I-b selected from those depicted in Table 1, above, or a pharmaceutically acceptable salt thereof. In some embodiments, the present disclosure provides a compound of formula I-b selected from those depicted in Table 1, above.

In some embodiments, the present disclosure comprises a compound of formula I-c selected from those depicted in Table 1, above, or a pharmaceutically acceptable salt, stereoisomer, or mixture of stereoisomers thereof. In some embodiments, the present disclosure provides a compound of formula I-c selected from those depicted in Table 1, above, or a pharmaceutically acceptable salt thereof. In some embodiments, the present disclosure provides a compound of formula I-c selected from those depicted in Table 1, above.

In some embodiments, the present disclosure comprises a compound of formula I-d selected from those depicted in Table 1, above, or a pharmaceutically acceptable salt, stereoisomer, or mixture of stereoisomers thereof. In some embodiments, the present disclosure provides a compound of formula I-d selected from those depicted in Table 1, above, or a pharmaceutically acceptable salt thereof. In some embodiments, the present disclosure provides a compound of formula I-d selected from those depicted in Table 1, above.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that is effective to measurably inhibit a PI3Kα protein kinase, or a mutant thereof, in a biological sample or in a patient. In certain embodiments, the amount of compound in compositions of this disclosure is such that it is effective to measurably inhibit a PI3Kα protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a PI3Kα protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day.

It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Ale^{YA}nder T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated allyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the degradation and/or inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3K6, and PI3Ky. In some embodiments, the kinase inhibited by the compounds and compositions described herein is a PI3Kα.

In some embodiments, the kinase inhibited by the compounds and compositions described herein is a PI3Kα containing at least one of the following mutations: H1047R, E542K, and E545K. In some embodiments, the kinase inhibited by the compounds and compositions described herein is a PI3Kα containing a H1047R mutation. In some embodiments, the kinase inhibited by the compounds and compositions described herein is a PI3Kα containing a E542K mutation. In some embodiments, the kinase inhibited by the compounds and compositions described herein is a PI3Kα containing a E545K mutation.

In some embodiments, the kinase inhibited by the compounds and compositions described herein is a PI3Kα containing at least one of the following mutations: E542X, E545X, Q546X, H1047X, and G1049X, wherein X is any amino acid besides its wildtype. In some embodiments, the kinase inhibited by the compounds and compositions described herein is a PI3Kα containing an E542X mutation, wherein X is any amino acid besides its wildtype. In some embodiments, the kinase inhibited by the compounds and compositions described herein is a PI3Kα containing an E545X mutation, wherein X is any amino acid besides its wildtype. In some embodiments, the kinase inhibited by the compounds and compositions described herein is a PI3Kα containing an Q546X mutation, wherein X is any amino acid besides its wildtype. In some embodiments, the kinase inhibited by the compounds and compositions described herein is a PI3Kα containing an H1047X mutation, wherein X is any amino acid besides its wildtype. In some embodiments, the kinase inhibited by the compounds and compositions described herein is a PI3Kα containing an G1049X mutation, wherein X is any amino acid besides its wildtype.

In some embodiments, the kinase inhibited by the compounds and compositions described herein is a PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the kinase inhibited by the compounds and compositions described herein is a PI3Kα containing an E542K mutation. In some embodiments, the kinase inhibited by the compounds and compositions described herein is a PI3Kα containing an E542Q mutation. In some embodiments, the kinase inhibited by the compounds and compositions described herein is a PI3Kα containing an E545A mutation. In some embodiments, the kinase inhibited by the compounds and compositions described herein is a PI3Kα containing an E545G mutation. In some embodiments, the kinase inhibited by the compounds and compositions described herein is a PI3Kα containing an E545K mutation. In some embodiments, the kinase inhibited by the compounds and compositions described herein is a PI3Kα containing an E545Q mutation. In some embodiments, the kinase inhibited by the compounds and compositions described herein is a PI3Kα containing an Q546E mutation. In some embodiments, the kinase inhibited by the compounds and compositions described herein is a PI3Kα containing an Q546K mutation. In some embodiments, the kinase inhibited by the compounds and compositions described herein is a PI3Kα containing an Q546L mutation. In some embodiments, the kinase inhibited by the compounds and compositions described herein is a PI3Kα containing an Q546P mutation. In some embodiments, the kinase inhibited by the compounds and compositions described herein is a PI3Kα containing an Q546R mutation. In some embodiments, the kinase inhibited by the compounds and compositions described herein is a PI3Kα containing an H1047R mutation. In some embodiments, the kinase inhibited by the compounds and compositions described herein is a PI3Kα containing an H1047L mutation. In some embodiments, the kinase inhibited by the compounds and compositions described herein is a PI3Kα containing an H1047Y mutation. In some embodiments, the kinase inhibited by the compounds and compositions described herein is a PI3Kα containing an G1049R mutation. In some embodiments, the kinase inhibited by the compounds and compositions described herein is a PI3Kα containing an G1049S mutation.

Compounds or compositions of the disclosure can be useful in applications that benefit from degradation and/or inhibition of PI3K enzymes. For example, PI3K inhibitors of the present disclosure are useful for the treatment of cellular proliferative diseases generally. Compounds or compositions of the disclosure can be useful in applications that benefit from inhibition of PI3Kα enzymes. For example, PI3Kα inhibitors of the present disclosure are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

The activity of a compound utilized in this disclosure as a degrader and/or inhibitor of a PI3K kinase, for example, a PI3Kα, or a mutant thereof, may be assayed in vitro, in vivo or in a cell line. In vitro assays include assays that determine inhibition of either the phosphorylation activity and/or the subsequent functional consequences, or ATPase activity of an activated PI3Kα, or a mutant thereof. Alternative in vitro assays quantitate the ability of the inhibitor to bind to a a PI3Kα. Inhibitor binding may be measured by radiolabeling the inhibitor prior to binding, isolating the inhibitor/PI3Kα complex and determining the amount of radiolabel bound. Alternatively, inhibitor binding may be determined by running a competition experiment where new inhibitors are incubated with a PI3Kα bound to known radioligands. Representative in vitro and in vivo assays useful in assaying a PI3Kα inhibitor include those described and disclosed in the patent and scientific publications described herein. Detailed conditions for assaying a compound utilized in this disclosure as an inhibitor of a PI3Kα, or a mutant thereof, are set forth in the Examples below.

### Treatment of Disorders

Provided compounds are inhibitors of PI3Kα and are therefore useful for treating one or more disorders associated with activity of PI3Kα or mutants thereof. Thus, in certain embodiments, the present disclosure provides a method of treating a PI3Kα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a PI3Kα-mediated disorder in a subject comprising administering a therapeutically effective amount of a compound of the present disclosure, or a pharmaceutically acceptable composition thereof, to a subject in need thereof. In some embodiments, the subject has a mutant PI3Kα.

In some embodiments, the subject has PI3Kα containing at least one of the following mutations: H1047R, E542K, and E545K. In some embodiments, the subject has PI3Kα containing a H1047R mutation. In some embodiments, the subject has PI3Kα containing a E542K mutation. In some embodiments, the subject has PI3Kα containing a E545K mutation.

In some embodiments, the subject has PI3Kα containing at least one of the following mutations: E542X, E545X, Q546X, H1047X, and G1049X, wherein X is any amino acid besides its wildtype. In some embodiments, the subject has PI3Kα containing an E542X mutation, wherein X is any amino acid besides its wildtype. In some embodiments, the subject has PI3Kα containing an E545X mutation, wherein X is any amino acid besides its wildtype. In some embodiments, the subject has PI3Kα containing an Q546X mutation, wherein X is any amino acid besides its wildtype. In some embodiments, the subject has PI3Kα containing an H1047X mutation, wherein X is any amino acid besides its wildtype. In some embodiments, the subject has PI3Kα containing an G1049X mutation, wherein X is any amino acid besides its wildtype.

In some embodiments, the subject has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the subject has PI3Kα containing an E542K mutation. In some embodiments, the subject has PI3Kα containing an E542Q mutation. In some embodiments, the subject has PI3Kα containing an E545A mutation. In some embodiments, the subject has PI3Kα containing an E545G mutation. In some embodiments, the subject has PI3Kα containing an E545K mutation. In some embodiments, the subject has PI3Kα containing an E545Q mutation. In some embodiments, the subject has PI3Kα containing an Q546E mutation. In some embodiments, the subject has PI3Kα containing an Q546K mutation. In some embodiments, the subject has PI3Kα containing an Q546L mutation. In some embodiments, the subject has PI3Kα containing an Q546P mutation. In some embodiments, the subject has PI3Kα containing an Q546R mutation. In some embodiments, the subject has PI3Kα containing an H1047R mutation. In some embodiments, the subject has PI3Kα containing an H1047L mutation. In some embodiments, the subject has PI3Kα containing an H1047Y mutation. In some embodiments, the subject has PI3Kα containing an G1049R mutation. In some embodiments, the subject has PI3Kα containing an G1049S mutation.

As used herein, the term “PI3Kα-mediated” disorders, diseases, and/or conditions means any disease or other deleterious condition in which PI3Kα or a mutant thereof is known to play a role. Accordingly, another embodiment of the present disclosure relates to treating or lessening the severity of one or more diseases in which PI3Kα, or a mutant thereof, is known to play a role. Such PI3Kα-mediated disorders include, but are not limited to, cellular proliferative disorders (e.g. cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K. In some embodiments, the PI3Kα-mediated disorder is a disorder mediated by a PI3Kα according to one of the embodiments above describing PI3Kα mutants that a subject has.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K. In some embodiments, the subject has PI3Kα containing a mutation as described in embodiments above.

In some embodiments, the method of treatment comprises the steps of: i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K. In some embodiments, the subject has PI3Kα containing a mutation as described in embodiments above.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα.

In some embodiments, the cellular proliferative disease has PI3Kα containing at least one of the following mutations: H1047R, E542K, and E545K. In some embodiments, the cancer has PI3Kα containing at least one of the following mutations: H1047R, E542K, and E545K. In some embodiments, the breast cancer has PI3Kα containing at least one of the following mutations: H1047R, E542K, and E545K. In some embodiments, the ovarian cancer has PI3Kα containing at least one of the following mutations: H1047R, E542K, and E545K. In some embodiments, the cellular proliferative disease has a mutant PI3Kα according to one of the embodiments above describing PI3Kα mutants that a subject has.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85a that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85a that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present disclosure, may be administered using any amount and any route of administration effective for treating or lessening the severity of the disorder (e.g. a proliferative disorder). The e^{YA}ct amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months.

In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Degradation of Protein Kinases

In some embodiments, the disclosure provides compounds that modulate targeted ubiquitination and degradation of a PI3K, or a mutant thereof. In some embodiments, the compounds described herein can inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

According to one embodiment, the disclosure relates to a method of degrading protein kinases in a biological sample comprising the step of contacting said biological sample with a compound of this disclosure, or a composition comprising said compound. According to another embodiment, the disclosure relates to a method of degrading PI3K, or a mutant thereof, in a biological sample comprising the step of contacting said biological sample with a compound of this disclosure, or a composition comprising said compound. According to another embodiment, the disclosure relates to a method of degrading PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a H1047R mutation. In some embodiments, the PI3Kα contains a E542K mutation. In some embodiments, the PI3Kα contains a E545K mutation.

In some embodiments, the PI3Kα contains at least one of the following mutations: E542X, E545X, Q546X, H1047X, and G1049X, wherein X is any amino acid besides its wildtype. In some embodiments, the PI3Kα contains an E542X mutation, wherein X is any amino acid besides its wildtype. In some embodiments, the PI3Kα contains an E545X mutation, wherein X is any amino acid besides its wildtype. In some embodiments, the PI3Kα contains an Q546X mutation, wherein X is any amino acid besides its wildtype. In some embodiments, the PI3Kα contains an H1047X mutation, wherein X is any amino acid besides its wildtype. In some embodiments, the PI3Kα contains an G1049X mutation, wherein X is any amino acid besides its wildtype.

In some embodiments, the PI3Kα contains at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the PI3Kα contains an E542K mutation. In some embodiments, the PI3Kα contains an E542Q mutation. In some embodiments, the PI3Kα contains an E545A mutation. In some embodiments, the PI3Kα contains an E545G mutation. In some embodiments, the PI3Kα contains an E545K mutation. In some embodiments, the PI3Kα contains an E545Q mutation. In some embodiments, the PI3Kα contains an Q546E mutation. In some embodiments, the PI3Kα contains an Q546K mutation. In some embodiments, the PI3Kα contains an Q546L mutation. In some embodiments, the PI3Kα contains an Q546P mutation. In some embodiments, the PI3Kα contains an Q546R mutation. In some embodiments, the PI3Kα contains an H1047R mutation. In some embodiments, the PI3Kα contains an H1047L mutation. In some embodiments, the PI3Kα contains an H1047Y mutation. In some embodiments, the PI3Kα contains an G1049R mutation. In some embodiments, the PI3Kα contains an G1049S mutation.

In another embodiment, the disclosure provides a method of selectively degrading PI3Kα over one or both of PI3K6 and PI3Ky. In some embodiments, a compound of the present disclosure is more than 5-fold selective over PI3K6 and PI3Ky. In some embodiments, a compound of the present disclosure is more than 10-fold selective over PI3K6 and PI3Ky. In some embodiments, a compound of the present disclosure is more than 50-fold selective over PI3K6 and PI3Ky. In some embodiments, a compound of the present disclosure is more than 100-fold selective over PI3K6 and PI3Ky. In some embodiments, a compound of the present disclosure is more than 200-fold selective over PI3K6 and PI3Ky. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

In another embodiment, the disclosure provides a method of selectively degrading a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Degradation of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of degrading protein kinase activity in a patient comprising the step of administering to said patient a compound of the present disclosure, or a composition comprising said compound.

According to another embodiment, the disclosure relates to a method of degrading PI3K, or a mutant thereof, in a patient comprising the step of administering to said patient a compound of the present disclosure, or a composition comprising said compound. In some embodiments, the disclosure relates to a method of degrading PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

### Inhibition of Protein Kinases

In some embodiments, the disclosure provides bifunctional compounds that bind to one or more sites of a PI3K, or a mutant thereof. In some embodiments, the disclosure provides bifunctional compounds that bind to at least one site of a PI3K, or a mutant thereof. In some embodiments, the disclosure provides bifunctional compounds that bind to one site of a PI3K, or a mutant thereof. In some embodiments, the disclosure provides bifunctional compounds that bind to at least two sites of a PI3K, or a mutant thereof. In some embodiments, the disclosure provides bifunctional compounds that bind to two sites of a PI3K, or a mutant thereof.

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a PI3K, or a mutant thereof, in a biological sample comprising the step of contacting said biological sample with a compound of this disclosure, or a composition comprising said compound. According to another embodiment, the disclosure relates to a method of inhibiting activity of PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

In another embodiment, the disclosure provides a method of selectively inhibiting PI3Kα over one or both of PI3K6 and PI3Ky. In some embodiments, a compound of the present disclosure is more than 5-fold selective over PI3K6 and PI3Ky. In some embodiments, a compound of the present disclosure is more than 10-fold selective over PI3K6 and PI3Ky. In some embodiments, a compound of the present disclosure is more than 50-fold selective over PI3K6 and PI3Ky. In some embodiments, a compound of the present disclosure is more than 100-fold selective over PI3K6 and PI3Ky. In some embodiments, a compound of the present disclosure is more than 200-fold selective over PI3K6 and PI3Ky. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

In another embodiment, the disclosure provides a method of selectively inhibiting a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a PI3K, or a mutant thereof, in a patient comprising the step of administering to said patient a compound of the present disclosure, or a composition comprising said compound. In some embodiments, the disclosure relates to a method of inhibiting activity of PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

According to another embodiment, the present disclosure provides a method of inhibiting signaling activity of PI3Kα, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting PI3Kα signaling activity in a subject, comprising administering a therapeutically effective amount of a compound according to the present disclosure, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the PI3Kα contains at least one of the following mutations: H1047X, E542X, and E545X, wherein X is a any amino acid residue other than histidine or glutamic acid, respectively. In some embodiments, the PI3Kα contains at least one of the following mutations: H1047R, H1047L, E542K, and E545K. In some embodiments, the PI3Kα contains a mutation according the one of the embodiments described above.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the disclosure in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the disclosure in combination with an inhibitor of Her1/EGFR. In certain embodiments, the method of treatment comprises administering the compound or composition of the disclosure in combination with one or more of tarceva, gefitinib, and erbitux. In certain embodiments, the method of treatment comprises administering the compound or composition of the disclosure in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the disclosure in combination with a treatment for a hormone-dependent cancer. In certain embodiments, the method of treatment comprises administering the compound or composition of the disclosure in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Abl employs the Ras-Raf kinase pathway to elicit its effects. In addition, inhibiting more than one kinase in the same pathway provides additional protection against resistance-conferring mutations.

Accordingly, in another aspect, the compounds and compositions of the disclosure are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see e.g. Ultimo et al. Oncotarget (2017) 8 (14) 23213-23227.): e.g. imatinib, inilotinib, nilotinib, dasatinib, bosutinib, ponatinib, bafetinib, danusertib, saracatinib, PF03814735; ALK inhibitors (see e.g. Yang et al. Tumour Biol. (2014) 35 (10) 9759-67): e.g. crizotinib, NVP-TAE684, ceritinib, alectinib, brigatinib, entrecinib, lorlatinib; BRAF inhibitors (see e.g. Silva et al. Mol. Cancer Res. (2014) 12, 447-463): e.g. vemurafenib, dabrafenib; FGFR inhibitors (see e.g. Packer et al. Mol. Cancer Ther. (2017) 16(4) 637-648): e.g. infigratinib, dovitinib, erdafitinib, TAS-120, pemigatinib, BLU-554, AZD4547; FLT3 inhibitors: e.g. sunitinib, midostaurin, tanutinib, sorafenib, lestaurtinib, quizartinib, and crenolanib; MEK Inhibitors (see e.g. Jokinen et al. Ther. Adv. Med. Oncol. (2015) 7(3) 170-180): e.g. trametinib, cobimetinib, binimetinib, selumetinib; ERK inhibitors: e.g. ulixertinib, MK 8353, LY 3214996; KRAS inhibitors: e.g. AMG-510, MRTX849, ARS-3248; Tyrosine kinase inhibitors (see e.g. Makhov et al. Mol. Cancer. Ther. (2012) 11(7) 1510-1517): e.g. erlotinib, linifanib, sunitinib, pazopanib; Epidermal growth factor receptor (EGFR) inhibitors (see e.g. She et al. BMC Cancer (2016) 16, 587): gefitnib, osimertinib, cetuximab, panitumumab; HER2 receptor inhibitors (see e.g. Lopez et al. Mol. Cancer Ther. (2015) 14(11) 2519-2526): e.g. trastuzumab, pertuzumab, neratinib, lapatinib, lapatinib; MET inhibitors (see e.g. Hervieu et al. Front. Mol. Biosci. (2018) 5, 86): e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents (see e.g. Wang et al. Cell Death & Disease (2018) 9, 739): e.g. o^{YA}liplatin, carboplatin, cisplatin; Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors (see e.g. Chen et al. Oncotarget (2014) 5 (9). 2372-2389): e.g. tanespimycin; Hedgehog antagonists (see e.g. Chaturvedi et al. Oncotarget (2018) 9 (24), 16619-16633): e.g. vismodegib; Proteasome inhibitors (see e.g. Lin et al. Int. J. Oncol. (2014) 44 (2), 557-562): e.g. bortezomib; PI3K inhibitors: e.g. pictilisib, dactolisib, alpelisib, buparlisib, taselisib, idelalisib, duvelisib, umbralisib; SHP2 inhibitors (see e.g. Sun et al. Am. J. Cancer Res. (2019) 9 (1), 149-159: e.g. SHP099, RMC-4550, RMC-4630); BCL-2 inhibitors (see e.g. Bojarczuk et al. Blood (2018) 133 (1), 70-80): e.g. venetoclax; Aromatase inhibitors (see e.g. Mayer et al. Clin. Cancer Res. (2019) 25 (10), 2975-2987): exemestane, letrozole, anastrozole, fulvestrant, tamoxifen; mTOR inhibitors (see e.g. Woo et al. Oncogenesis (2017) 6, e385): e.g. temsirolimus, ridaforolimus, everolimus, sirolimus; CTLA-4 inhibitors (see e.g. O'Donnell et al. (2018) 48, 91-103): e.g. tremelimumab, ipilimumab; PD1 inhibitors (see O'Donnell, supra): e.g. nivolumab, pembrolizumab; an immunoadhesin; Other immune checkpoint inhibitors (see e.g. Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g. pidilizumab, AMP-224; PDL1 inhibitors (see e.g. O'Donnell supra): e.g. MSB0010718C; YW243.55.S70, MPDL3280A; MEDI-4736, MSB-0010718C, or MDX-1105; Histone deacetylase inhibitors (HDI, see e.g. Rahmani et al. Clin. Cancer Res. (2014) 20(18), 4849-4860): e.g. vorinostat; Androgen Receptor inhibitors (see e.g. Thomas et al. Mol. Cancer Ther. (2013) 12(11), 2342-2355): e.g. enzalutamide, abiraterone acetate, orteronel, galeterone, seviteronel, bicalutamide, flutamide; Androgens: e.g. fluoxymesterone; CDK4/6 inhibitors (see e.g. Gul et al. Am. J. Cancer Res. (2018) 8(12), 2359-2376): e.g. alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.)

The structure of the active compounds identified by code numbers, generic or trade names may be taken from the actual edition of the standard compendium “The Merck Index” or from databases, e.g. Patents International (e.g. IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### List of Abbreviations

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
Boc: tert-butyloxycarbonyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
Fmoc: fluorenylmethoxycarbonyl
h or hrs: hours
HATU: 1-[Bis(dimethylamino)methylene]-1H-1,2,3-triazolo[4,5-b]pyridinium 3-oxide hexafluorophosphate
HOBt: 1H-1,2,3-Benzotriazol-1-ol
HPLC: high pressure liquid chromatography
LCMS or LC-MS: liquid chromatography-mass spectrometry
Ms: methanesulfonyl
MS: mass spectrometry
N: normality
NBS: N-bromosuccinimide
NMM: N-methyl morpholine
NMR: nuclear magnetic resonance
Pd(dppf)Cl₂: 1,1′-Bis(diphenylphosphino)ferrocene]palladium(II) dichloride
PE: petroleum ether
PMB: p-methoxybenzyl
PyAOP: (7-Azabenzotriazol-1-yloxy)tripyrrolidinophosphonium hexafluorophosphate
rt or RT: room temperature
sat: saturated
T₃P: propyl phosphonic anhydride
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultra violet

### LC/MS Methods Employed in Characterization of Examples

Reversed-phase analytical LCMS was performed on Nexara Shimadzu system coupled with Shimadzu LCMS-2020 system (Method A, F), Agilent 1100 series coupled with Agilent LC/MSDVL (Method B), or an Agilent 1200 series coupled with API 3200 (Method C, G, H, I) according to the following methods:

Method A: With a hold of 5% B till 0.5 min followed by linear Gradient of 5% to 95% B over 0.5 min, with 0.5 min hold at 95% B followed by 0.5 min linear gradient from 95% to 5% B with 1 min hold at 5% of B;
UV visualization at 210 nm
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile.
Method B: With a hold of 30% B till 0.5 min followed by linear Gradient of 30% to 95% B over 1.0 min, with 0.9 min hold at 95% B followed by 0.1 min linear gradient from 95% to 30% B with 0.5 min hold at 30% of B;
UV visualization at 210 nm
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile
Method C: Linear Gradient of 10%-20% of B over 0.5 min, followed by 1.0 min linear gradient of 20%-95% B, followed by linear gradient of 95%-10% over 0.5 min, with 0.5 min hold at 10% of B;
UV visualization at TWC (Total wavelength chromatogram from 190-400 nm)
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile.
Method D: With a hold of 30% B till 0.5 min followed by linear Gradient of 30% to 95% of B over 1.0 min, with 0.9 min hold at 95% B followed by 0.1 min linear gradient from 95% to 30% B with 0.5 min hold at 30% of B;
UV visualization at TWC (Total wavelength chromatogram from 190-400 nm)
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile
Method E: With a hold of 30% B till 0.5 min followed by linear Gradient of 30% to 95% of B over 1.0 min, with 0.9 min hold at 95% B followed by 0.1 min linear gradient of 95% to 100% B, followed by 0.5 min linear gradient from 100% to 30% B with 1.0 min hold at 30% of B;
UV visualization at TWC (Total wavelength chromatogram from 190-400 nm)
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile.
Method F: Linear Gradient of 5% to 30% over 1.0 min, followed by linear gradient of 30% to 95% over 2.0 min with 0.5 min hold at 95% B followed by 0.1 min linear gradient from 95% to 5% B with 2.0 min hold at 5% of B;
UV visualization at 210 nm
Column: Kinetex 2.6μ, 100×4.6 mm, C18 at 40° C.
Flow rate: 1.4 mL/min
Solvent A: 0.1% formic acid, 99.9% water
Solvent B: Acetonitrile
Method G: Linear Gradient of 10% to 50% over 0.2 min, followed by linear gradient of 50% to 95% over 0.8 min with 1.5 min hold at 95% B followed by 0.4 min linear gradient from 95% to 50% B followed by 0.3 min linear gradient from 50% to 10% B with 0.8 min hold at 10% of B;
UV visualization at TWC (Total wavelength chromatogram from 190-400 nm)
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile.
Method H: With a hold of 10% B till 0.2 min Linear Gradient of 10% to 95% over 0.8 min, With a hold of 95% B till 2.0 min followed by linear gradient of 95% to 10% over 0.1 min with 0.9 min hold at 10%;
UV visualization at TWC (Total wavelength chromatogram from 190-400 nm)
Column: Mercury MS Synergi Max RP 2.5μ, 20×4.0 mm, C12 at 30° C.
Flow rate: 1.2 mL/min
Solvent A: 0.1% formic acid, 99.9% water
Solvent B: Acetonitrile.
Method I: Linear Gradient of 20% to 50% over 0.2 min, followed by linear gradient of 50% to 95% over 0.8 min with 1.5 min hold at 95% B followed by 0.4 min linear gradient from 95% to 50% B followed by 0.3 min linear gradient from 50% to 20% B with 0.8 min hold at 20% of B;
UV visualization at TWC (Total wavelength chromatogram from 190-400 nm)
Column: Mercury MS Synergi Max RP 2.5μ, 20×4.0 mm, C12 at 30° C.
Flow rate: 2.0 mL/min
Solvent A: 0.1% formic acid, 99.9% water
Solvent B: Acetonitrile.

### HPLC Methods Employed in Characterization of Examples

Reversed-phase analytical HPLC was performed on Agilent 1100 series (Method E, Fand G), Agilent 1200 series (Method B), or Shimadzu SPD-M20A (Method A, C, D, H, I, J, K, L, M, N and O) according to the following methods:
Method A: Linear Gradient of 10% to 20% of B over 2.0 min, followed by 8.0 min linear gradient of 20% to 70% B, followed by 3 min linear gradient of 70% to 100%, with 2.0 min hold at 100% of B followed by linear gradient from 100% to 60% over 2.0 min, followed by linear gradient from 60% to 20% over 1 min, followed by linear gradient of 20% to 10% over 2 min;
UV visualization at 210 nm
Column: WATERS XBRIDGE C18 (150 mm×4.6 mm, 5 micon) at 40° C.
Flow rate: 1.0 mL/min
Solvent A: 0.05% NH4OH, 99.95% water
Solvent B: Acetonitrile.
Method B: With a hold of 05% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Zorbax, Eclipse, XDB-C18 (150 mm×4.6 mm, 5 micon) at 40° C.
Flow rate: 1.0 mL/min
Solvent A: 0.01% TFA, 99.99% water
Solvent B: 50% Acetonitrile, 50% Methanol.
Method C: Linear Gradient of 30% to 70% of B over 1.0 min, followed by 5.0 min linear gradient of 70% to 100% B, with 2.0 min hold at 100% of B followed by linear gradient from 100% to 30% over 2.0 min, with 2.0 min hold at 30% of B;
UV visualization at 210 nm
Column: Zorbax, Eclipse, XDB-C18 (150 mm×4.6 mm, 5 micon) at 40° C.
Flow rate: 1.0 mL/min
Solvent A: 0.01% TFA, 99.99% water
Solvent B: Acetonitrile.
Method D: Linear Gradient of 30% to 70% of B over 1.0 min, followed by 5.0 min linear gradient of 70% to 100% B, with 2.0 min hold at 100% of B followed by linear gradient from 100% to 30% over 2.0 min, with 2.0 min hold at 30% of B;
UV visualization at 210 nm
Column: Kinetex, C18 (150 mm×4.6 mm, 5 micon) at 40° C.
Flow rate: 1.0 mL/min
Solvent A: 0.01% TFA, 99.99% water
Solvent B: Acetonitrile
Method E: With a hold of 5% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Zorbax, Eclipse, XDB-C18 (150 mm×4.6 mm, 5 micon) at 40° C.
Flow rate: 1.0 mL/min
Solvent A: 0.01% TFA, 99.99% water
Solvent B: Acetonitrile.
Method F: Linear Gradient of 30% to 40% B over 1.0 min, followed by linear gradient of 40% to 90% B over 4.0 min, followed by linear gradient of 90% to 100% of B over 2 min, with 2.0 min hold at 100% B followed by 1.0 min linear gradient from 100% to 30% B with 2.0 min hold at 30% of B;
UV visualization at 210 nm
Column: Zorbax, Eclipse, XDB-C18 (150 mm×4.6 mm, 5 micon) at 40° C.
Flow rate: 1.0 mL/min
Solvent A: 0.01% TFA, 99.99% water
Solvent B: Acetonitrile
Method G: Linear Gradient of 30% to 40% B over 1.0 min, followed by linear gradient of 40% to 90% B over 4.0 min, followed by linear gradient of 90% to 100% of B over 2 min, with 2.0 min hold at 100% B followed by 1.0 min linear gradient from 100% to 30% B with 2.0 min hold at 30% of B;
UV visualization at 210 nm
Column: Waters Xbridge, C18 (150 mm×4.6 mm, 5 micon), at 40° C.
Flow rate: 1.0 mL/min
Solvent A: 0.01% TFA, 99.99% water
Solvent B: Acetonitrile.
Method H: Linear Gradient of 30% to 70% of B over 1.0 min, followed by 5.0 min linear gradient of 70% to 100% B, with 2.0 min hold at 100% of B followed by linear gradient from 100% to 30% over 2.0 min, with 2.0 min hold at 30% of B;
UV visualization at 210 nm
Column: Waters Xbridge, C18 (150 mm×4.6 mm, 5 micon), at 40° C.
Flow rate: 1.0 mL/min
Solvent A: Water
Solvent B: Acetonitrile.
Method I: With a hold of 05% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Waters Xbridge, C18 (150 mm×4.6 mm, 5 micon), at 40° C.
Flow rate: 1.0 mL/min
Solvent A: Water
Solvent B: Acetonitrile
Method J: Linear Gradient of 30% to 70% of B over 1.0 min, followed by 5.0 min linear gradient of 70% to 100% B, with 2.0 min hold at 100% of B followed by linear gradient from 100% to 30% over 2.0 min, with 2.0 min hold at 30% of B;
UV visualization at 210 nm
Column: Waters Xbridge, C18 (150 mm×4.6 mm, 5 micon), at 40° C.
Flow rate: 1.0 mL/min
Solvent A: 5 mM Ammonium acetate in water
Solvent B: Acetonitrile.
Method K: With a hold of 05% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Waters Xbridge, C18 (150 mm×4.6 mm, 5 micon), at 40° C.
Flow rate: 1.0 mL/min
Solvent A: 5 mM Ammonium acetate in water
Solvent B: Acetonitrile
Method L: With a hold of 05% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Waters Xbridge, C18 (150 mm×4.6 mm, 5 micon), at 40° C.
Flow rate: 1.0 mL/min
Solvent A: 0.05% NH₄OH solution (28% to 30% NH₃ basis), 99.95% water
Solvent B: Acetonitrile.
Method M: Linear Gradient of 30% to 70% of B over 1.0 min, followed by 5.0 min linear gradient of 70% to 100% B, with 2.0 min hold at 100% of B followed by linear gradient from 100% to 30% over 2.0 min, with 2.0 min hold at 30% of B;
UV visualization at 210 nm
Column: Waters Xbridge, C18 (150 mm×4.6 mm, 5 micon), at 40° C.
Flow rate: 1.0 mL/min
Solvent A: 0.05% NH₄OH solution (28% to 30% NH₃ basis), 99.95% water
Solvent B: Acetonitrile
Method N: Linear Gradient of 10% to 20% over 2.0 min, followed by linear gradient of 20% to 60% over 8.0 min, followed by linear gradient of 60% to 100% over 5.0 min with 3.0 min hold at 100% B followed by 2.0 min linear gradient from 100% to 10% B;
UV visualization at 210 nm
Column: Kinetex, C18 (150 mm×4.6 mm, 5 micon) at 40° C.
Flow rate: 1.0 mL/min
Solvent A: 0.01% TFA, 99.99% water
Solvent B: Acetonitrile.
Method O: With a hold of 05% B till 1.0 min followed by linear Gradient of 05% to 100% B over 5.0 min, with 2.0 min hold at 100% B followed by 2.0 min linear gradient from 100% to 5% B with 2.0 min hold at 05% of B;
UV visualization at 210 nm
Column: Kinetex, C18 (150 mm×4.6 mm, 5 micon) at 40° C.
Flow rate: 1.0 mL/min
Solvent A: 0.01% TFA, 99.99% water
Solvent B: 50% Acetonitrile, 50% Methanol.

### Preparative HPLC Methods Employed in Purification of Examples

Reversed-phase preparative HPLC was performed on Agilent 1200 Series according to the following methods:
Method A: Linear Gradient of 50% to 60% B over 2.0 min, followed by linear gradient of 60% to 70% B over 6.0 min;
UV visualization at 210 nm
Column: X SELECT (250 mm×19 mm), 5.0μ
Flow rate: 18.0 mL/min
Solvent A: Water
Solvent B: Acetonitrile
Method B: Linear Gradient of 30% to 35% B over 2.0 min, followed by linear gradient of 35% to 60% B over 6.0 min;
UV visualization at 210 nm
Column: Kinetex EVO (250 mm×21.2 mm), 5.0μ
Flow rate: 18.0 mL/min
Solvent A: 0.1% HCOOH IN WATER
Solvent B: Acetonitrile
Method C: Linear Gradient of 30% to 40% B over 2.0 min, followed by linear gradient of 40% to 60% B over 6.0 min;
UV visualization at 210 nm
Column: LUNA Phenomenex (250 mm×21.2 mm), 5.0μ
Flow rate: 18.0 mL/min
Solvent A: 0.1% HCOOH IN WATER
Solvent B: Acetonitrile
Method D: Linear Gradient of 30% to 40% B over 2.0 min, followed by linear gradient of 40% to 70% B over 8.0 min;
UV visualization at 210 nm
Column: ATLANTIS (C18, 19 mm×250 mm),
Flow rate: 18.0 mL/min
Solvent A: 0.1% HCOOH IN WATER
Solvent B: Acetonitrile
Method E: Linear Gradient of 20% to 30% B over 2.0 min, followed by linear gradient of 30% to 60% B over 6.0 min;
UV visualization at 210 nm
Column: X-Bridge (150 mm×21.2 mm), 5.0μ
Flow rate: 15.0 mL/min
Solvent A: 0.1% HCOOH IN WATER
Solvent B: Acetonitrile
Method F: Linear Gradient of 25% to 30% B over 2.0 min, followed by linear gradient of 30% to 60% B over 8.0 min;
UV visualization at 210 nm
Column: XSELECT (C18, 19 mm×250 mm)
Flow rate: 18.0 mL/min
Solvent A: 0.1% HCOOH IN WATER
Solvent B: Acetonitrile
Method G: Linear Gradient of 25% to 35% B over 2.0 min, followed by linear gradient of 35% to 60% B over 8.0 min;
UV visualization at 210 nm
Column: ATLANTIS (C18, 19 mm×250 mm),
Flow rate: 18.0 mL/min
Solvent A: 0.1% HCOOH IN WATER
Solvent B: Acetonitrile
Method H: Linear Gradient of 10% to 20% B over 2.0 min, followed by linear gradient of 20% to 60% B over 8.0 min;
UV visualization at 210 nm
Column: ATLANTIS (C18, 19 mm×250 mm),
Flow rate: 18.0 mL/min
Solvent A: 0.1% HCOOH IN WATER
Solvent B: Acetonitrile
Method I: Linear Gradient of 25% to 30% B over 2.0 min, followed by linear gradient of 30% to 60% B over 6.0 min;
UV visualization at 210 nm
Column: Kinetex (150 mm×21.2 mm), 5.0μ
Flow rate: 18.0 mL/min
Solvent A: 0.1% HCOOH IN WATER
Solvent B: Acetonitrile
Method J: Linear Gradient of 20% to 25% B over 2.0 min, followed by linear gradient of 25% to 65% B over 6.0 min;
UV visualization at 210 nm
Column: X-Select (250 mm×21.2 mm), 5.0μ
Flow rate: 18.0 mL/min
Solvent A: 0.1% HCOOH IN WATER
Solvent B: Acetonitrile
Method K: Linear Gradient of 30% to 35% B over 2.0 min, followed by linear gradient of 35% to 60% B over 6.0 min;
UV visualization at 210 nm
Column: LUNA Phenomenex (250 mm×21.2 mm), 5.0μ
Flow rate: 18.0 mL/min
Solvent A: 0.1% HCOOH IN WATER
Solvent B: Acetonitrile
Method L: Linear Gradient of 40% to 45% B over 2.0 min, followed by linear gradient of 45% to 70% B over 6.0 min;
UV visualization at 210 nm
Column: LUNA Phenomenex (250 mm×21.2 mm), 5.0μ
Flow rate: 18.0 mL/min
Solvent A: WATER
Solvent B: Acetonitrile
Method M: Linear Gradient of 30% to 40% B over 2.0 min, followed by linear gradient of 40% to 50% B over 8.0 min;
UV visualization at 210 nm
Column: X BRIDGE (150 mm×20.0 mm), 5.0μ
Flow rate: 18.0 mL/min
Solvent A: WATER
Solvent B: Acetonitrile
Method N: Linear Gradient of 45% to 55% B over 2.0 min, followed by linear gradient of 55% to 85% B over 8.0 min;
UV visualization at 210 nm
Column: Kinetex EVO (250 mm×21.2 mm), 5.0μ
Flow rate: 18.0 mL/min
Solvent A: WATER
Solvent B: Acetonitrile
Method O: Linear Gradient of 20% to 30% B over 2.0 min, followed by linear gradient of 30% to 60% B over 6.0 min;
UV visualization at 210 nm
Column: Kinetex EVO (250 mm×21.2 mm), 5.0μ
Flow rate: 17.0 mL/min
Solvent A: 0.1% HCOOH IN WATER
Solvent B: Acetonitrile
Method P: Linear Gradient of 60% to 70% B over 2.0 min, followed by linear gradient of 70% to 90% B over 3.0 min;
UV visualization at 210 nm
Column: JUPITAR
Flow rate: 18.0 mL/min
Solvent A: 0.1% HCOOH IN WATER
Solvent B: Acetonitrile
Method Q: Linear Gradient of 30% to 40% B over 2.0 min, followed by linear gradient of 40% to 70% B over 6.0 min;
UV visualization at 210 nm
Column: Zorbax XDB (150 mm×21.2 mm), 5.0μ
Flow rate: 18.0 mL/min
Solvent A: 0.1% HCOOH IN WATER
Solvent B: Acetonitrile
Method R: Linear Gradient of 20% to 30% B over 2.0 min, followed by linear gradient of 30% to 70% B over 8.0 min;
UV visualization at 210 nm
Column: GEMINI NX C18 (250 mm×21.2 mm 5 μm)
Flow rate: 18.0 mL/min
Solvent A: 0.1% HCOOH IN WATER
Solvent B: Acetonitrile
Method S: Linear Gradient of 40% to 50% B over 2.0 min, followed by linear gradient of 50% to 70% B over 8.0 min;
UV visualization at 210 nm
Column: Zorbax XDB (150 mm×21.2 mm), 5.0μ
Flow rate: 18.0 mL/min
Solvent A: WATER
Solvent B: Acetonitrile
Method T: Linear Gradient of 10% to 20% B over 2.0 min, followed by linear gradient of 20% to 85% B over 6.0 min;
UV visualization at 210 nm
Column: YMC (C18, 19 mm×150 mm)
Flow rate: 15.0 mL/min
Solvent A: 0.1% HCOOH IN WATER
Solvent B: Acetonitrile

### Chiral Preparative HPLC Methods Employed in Purification of Examples

Chiral preparative HPLC was performed on Agilent 1260 Series according to the following methods:
Method A: ISOCRATIC:65(A):35(B)
UV visualization at 210 nm
Column: LUX CELLULOSE-4,250 MM×21.2 MM×5 MICRON
Flow rate: 15.0 mL/min
Solvent A: n-Hexane
Solvent B: 0.1% HCOOH IN EtOH: MeOH (1:1)
Method B: ISOCRATIC:60(A):40(B)
UV visualization at 210 nm
Column: LUX CELLULOSE-4,250 MM×21.2 MM×5 MICRON
Flow rate: 15.0 mL/min
Solvent A: n-Hexane
Solvent B: 0.1% HCOOH IN EtOH:MeOH (1:1)
Method C: ISOCRATIC:70(A):30(B)
UV visualization at 210 nm
Column: LUX CELLULOSE-4,250 MM×21.2 MM×5 MICRON
Flow rate: 15.0 mL/min
Solvent A: n-Hexane
Solvent B: 0.1% HCOOH IN EtOH:MeOH (1:1)
Method D: ISOCRATIC:75(A):25(B)
UV visualization at 210 nm
Column: CHIRALPAK IG—250 MM×20 MM×5 MICRON
Flow rate: 15.0 mL/min
Solvent A: n-Hexane
Solvent B: 0.1% HCOOH IN EtOH:MeOH (1:1)
Method E: ISOCRATIC:70(A):30(B)
UV visualization at 210 nm
Column: Regis Reflect i-Cellulose-C, 250 mm×21.1 mm×5 μm
Flow rate: 10.0 mL/min
Solvent A: n-Hexane
Solvent B: 0.1% TFA in EtOH:MeOH (80:20)
Method F: ISOCRATIC:70(A):30(B)
UV visualization at 210 nm
Column: Reis Reflect i-Cellulose-C, 250 mm×21.1 mm×5 μm
Flow rate: 15.0 mL/min
Solvent A: n-Hexane
Solvent B: 0.1% TFA in EtOH:MeOH (80:20)
Method G: ISOCRATIC:60(A):40(B)
UV visualization at 210 nm
Column: Regis(S,S) Whelk-01,250 mm×21.1 mm×5 μm
Flow rate: 15.0 mL/min
Solvent A: Acetonitrile
Solvent B: EtOH:MeOH (1:1)
Method H: ISOCRATIC:80(A):20(B)
UV visualization at 210 nm
Colum: Regis(S,S) Whelk-01, 250 mmλ21.1 mm, 5 μm
Flow rate: 15.0 mL/min
Solvent A: n-Hexane
Solvent B: EtOH:MeOH (1:1)
Method I: ISOCRATIC:80(A):20(B)
UV visualization at 210 nm
Column: CHIRALPAK-IG, 250 MM×21.1 MM×5 MICRON
Flow rate: 15.0 mL/min
Solvent A: n-Hexane
Solvent B: 0.1% HCOOH IN EtOH:MeOH (1:1)

¹H-NMR Instrument: ¹H NMR spectra were recorded on a Varian Mercury (300 MHz), Bruker (400 MHz—with autosampler) spectrometers with DMSO-D6 or CDCl₃ or CD₃OD as the solvents. Chemical shifts were reported in δ scale using tetramethyl silane (TMS, d 0.00) as internal standard and coupling constants (J) were reported in Hz. The standard abbreviations s, d, t, q, dd, dt, td and m were used to symbolize singlet, doublet, triplet, quartet, doublet of doublet, doublet of triplet, triplet of doublet and multiplet respectively.

### General Procedures

### Fluoro Displacement Reactions General Procedure A: DIPEA/DMF

To a sealed tube containing 4-Fluoro-thalidomide (1.0 equiv.) in DMF (10-15 volumes) was added free amine or amine HCl salt (1.0-1.2 equiv.), followed by DIPEA (2-4 equiv.). The reaction vessel was then sealed, and the reaction was stirred at 90° C. for 16 hours before it was cooled down to ambient temperature, diluted with H₂O and extracted with EtOAc (50 mL×2). The combined organic layers were washed with H₂O followed by brine, dried over Na₂SO₄, filtered, and was concentrated under reduced pressure to obtain the crude product, which was then purified by chromatography (silica gel or flash using pre-filled silica gel or neutral alumina cartridges) to provide the desired product.

### Fluoro Displacement Reactions General Procedure B: DIPEA/NMP

To a sealed tube containing 5-Fluoro-thalidomide (1.0 equiv.) in NMP (20-30 volumes) was added free amine or amine HCl salt (1.0 equiv.), followed by di isopropyl ethylamine (2-4 equiv.). The reaction vessel was then sealed, and the reaction was stirred at 130° C. for 1-1.5 hour in microwave condition before it was cooled down to ambient temperature, diluted with H₂O and extracted with EtOAc (50 mL×2). The combined organic layers were washed with H₂O followed by brine, dried over Na₂SO₄, filtered, and was concentrated under reduced pressure to obtain the crude product, which was then purified by chromatography (silica gel or flash using pre-filled silica gel or neutral alumina cartridges) to provide the desired product.

### Boc Deprotection General Procedure A: 4M HCl in Dioxane/DCM

4M HCl in dioxane (20-25 volume) was added in a dropwise manner to a solution of Boc protected compound in DCM (10-15 volume) at 0° C., and the resultant solution was then stirred at 0° C. to RT for 1-3 h. After completion of the reaction as monitored by TLC and LCMS analysis, the solvents were evaporated in vacuo and resulting residue was triturated with n-pentane (50 mL), followed by diethyl ether (20 mL) to afford the amine HCl salt, which was taken for further transformations without additional purifications.

### Boc Deprotection General Procedure B: TFA/DCM

To a solution of Boc protected compound in DCM (20-50 volume) was added TFA (8-10 volume) in a dropwise manner at 0° C., then stirred at 0° C. to RT for 1-4 h. After completion of the reaction as monitored by TLC and LCMS analysis, the solvents were evaporated in vacuo and resulting residue was triturated with n-pentane (50 mL), followed by diethyl ether (20 mL) to afford the amine TFA salt, which was taken for further transformations without additional purifications.

### Acid Amine Coupling General Procedure A: HATU, DIPEA, DMF

To a solution of a suitable carboxylic acid (1.0 equiv) in DMF (5-10 volume) was added HATU (1.2-2.0 equiv) followed by DIPEA (2.5-5.0 equiv) at 0° C. After stirring at 0° C. for 30 minutes, Boc deprotected amine HCl salt (1.0-1.3 equiv) in DMF (5-10 volume) was added to this mixture in a drop wise manner. The resultant reaction mixture was stirred at RT for 5-16 h. After completion of the reaction as monitored by TLC and LCMS analysis, the reaction mixture was then partitioned between a mixture of DCM and MeOH (19:1, 100 volume) and ice-cold water (100 volume) and extracted twice more with the same solvent mixture (100 volumes). The combined organic layers were washed with brine (100 volume) and was dried over Na₂SO₄ and the solvents were removed under reduced pressure. The crude compound thus obtained was purified by chromatography (silica gel or flash using pre-filled silica gel, neutral alumina cartridges) to afford title compound.

### Acid Amine Coupling General Procedure B: PyAOP, DIPEA, DMF

To a solution of a suitable carboxylic acid (1.0 equiv) in DMF (5-10 volume) was added PyAOP (1.5-2.0 equiv) followed by DIPEA (2.5-5.0 equiv) at 0° C. After stirring at 0° C. for 30 minutes, Boc deprotected amine HCl salt (1.0-1.3 equiv) in DMF (5-10 volume) added to this mixture in a drop wise manner at 0° C. The resulting reaction mixture was stirred at RT for 3-12 h. After completion of the reaction as monitored by TLC and LCMS analysis, the reaction mixture was then partitioned between a mixture of DCM and MeOH (19:1, 100 volume) and ice-cold water (100 volume) and extracted twice more with the same solvent mixture (100 volumes). The combined organic layers were washed with brine (100 volume) and was dried over Na₂SO₄ and the solvents were removed under reduced pressure. The crude compound thus obtained was purified by chromatography (silica gel or flash using pre-filled silica gel, neutral alumina cartridges) to afford title compound.

### Acid Amine Coupling General Procedure C: EDCI, DMAP, NMP, DCM

To a solution of amine (1.00 eq) and acid (1.10 eq) in DCM (10 volume) and NMP (3 volume) was added EDCI (1.50 eq) and DMAP (0.20 eq). The mixture was stirred at RT for 48 h, when LCMS analysis have indicated the completion of the reaction. The solvents were removed under reduced pressure and the resulting residue was diluted with water (200 mL), solid separated out was collected by vacuum filtration. It was then triturated with petroleum ether/EtOAc (10:1, 200 mL), then the filter cake was washed with EtOAc (100 mL) to furnish the desired product in pure form, which was used for further transformation without additional purification.

### Acid Amine Coupling General Procedure D: EDCI, HOBt, DIPEA, DMF

To a solution of a suitable carboxylic acid (1.0 equiv) in DMF (5-10 volume) was added EDCI (1.5-2.0 equiv), HOBt (1.5-2.0 equiv), followed by DIPEA (3-5.0 equiv) at 0° C. After stirring at 0° C. for 30 minutes, Boc deprotected amine HCl salt (1.0-1.3 equiv) in DMF (5-10 volume) was added to this mixture in a drop wise manner at 0° C. The resultant reaction mixture was stirred at RT for 5-16 h. After completion of the reaction as monitored by TLC and LCMS analysis, the reaction mixture was then partitioned between a mixture of DCM and MeOH (19:1, 100 volume) and ice-cold water (100 volume) and extracted twice more with the same solvent mixture (100 volumes). The combined organic layers were washed with brine (100 volume) and was dried over Na₂SO₄ and the solvents were removed under reduced pressure. The crude compound thus obtained was purified by chromatography (silica gel or flash using pre-filled silica gel, neutral alumina cartridges) to afford title compound.

### Acid Amine Coupling General Procedure E: T₃P, DIPEA, DCM

To a solution of Boc deprotected amine HCl salt (1.0 equiv.) in DCM (50-100 volume) were added DIPEA (4-5.0 equiv.), suitable carboxylic acid (1.10-1.50 equiv.) at 0° C. After stirring at 0° C. for 30 minutes, T₃P (1.2-1.5 equiv.) was added to this mixture in a drop wise manner at 0° C. The resultant reaction mixture was stirred at RT for 4-12 h. After completion of the reaction as monitored by TLC and LCMS analysis, the reaction mixture was then partitioned between a mixture of DCM and MeOH (19:1, 100 volume) and ice-cold water (100 volume) and extracted twice more with the same solvent mixture (100 volumes). The combined organic layers were washed with brine (100 volume) and was dried over Na₂SO₄ and the solvents were removed under reduced pressure. The crude compound thus obtained was purified by chromatography (silica gel or flash using pre-filled silica gel, neutral alumina cartridges) to afford title compound.

### Acid Amine Coupling General Procedure F: EDCI, HOAt, NMM, DMSO

To a solution of a suitable carboxylic acid (1.0 equiv) in DMSO (10 volume) was added EDCI (1.5 equiv), HOAt (1.5 equiv), followed by NMM (4 equiv) at 0° C. After stirring at 0° C. for 30 minutes, Boc deprotected amine HCl salt (1 equiv) in DMSO (5 volume) was added to this mixture in a drop wise manner at 0° C. The resultant reaction mixture was stirred at RT for 5-16 h. After completion of the reaction as monitored by TLC and LCMS analysis, the reaction mixture was then partitioned between a mixture of DCM and MeOH (19:1, 100 volume) and ice-cold water (100 volume) and extracted twice more with the same solvent mixture (100 volumes). The combined organic layers were washed with brine (100 volume) and was dried over Na₂SO₄ and the solvents were removed under reduced pressure. The crude compound thus obtained was purified by chromatography (silica gel or flash using pre-filled silica gel, neutral alumina cartridges) to afford title compound.

Ester Hydrolysis General Procedure A: LiOH·H₂O, THF, MeOH, H₂O

To a solution of Ester intermediate (1.0 equiv) in THF/MeOH/H2O (4:1:1, 10 volume) was added LiOH·H₂O (2.0-3.0 equiv) at 0° C. The resulting reaction mixture was stirred at RT for 2-5 h. After completion of the reaction as monitored by TLC and LCMS analysis, the reaction mixture was concentrated in vacuo. The resulting residue was acidified to pH 5 to 6 with 2 N HCl solution and partitioned between EtOAc (100 volume) and ice-cold water (20 volume). Extraction was done twice with EtOAc (2×100 volume). The combined organic layers were washed with brine (10 volume) and was dried over Na₂SO₄ and the solvents were removed under reduced pressure to afford title compound.

Ester Hydrolysis General Procedure B: LiOH·H₂O, THF, H₂O

To a solution of Ester intermediate (1.0 equiv) in THF (10 volume) and H₂O (5 volume) was added LiOH·H₂O (1.0 equiv) at 25° C. The mixture was stirred at 25° C. for 2-24 h. The progress of the reaction was monitored by TLC and LCMS. The mixture was concentrated in vacuo. Then the mixture was poured into water (5 volume) and extracted with ethyl acetate (5 volume), adjusted the pH of the aqueous layer with HCl (2M) to 3. The precipitated white solid was filtered, and the filter cake was dried to afford title compound, which was used for further transformation without additional purification.

Tert-Butyl Ester Hydrolysis General Procedure A: 4M HCl in dioxane

A cooled 4M HCl in dioxane (60-80 volume) was added to tert-butyl ester intermediate (1.0 equiv) at 10° C. under inert atmosphere. The reaction mixture was allowed to stir for 3 to 5 h at RT. After completion of reaction (monitored by TLC and LCMS) evaporated the solvent in vacuo to dryness and resulting residue was triturated with pentane (20 mL), followed by diethyl ether (10 mL), to afford acid compound, which was used for further transformation without additional purification.

### Example Starting Material Preparation

Certain starting materials were synthesized according to the following methods.

### Preparation of 5-(benzo[b]thiophene-3-carboxamido)-6-(o-tolylamino)nicotinic acid

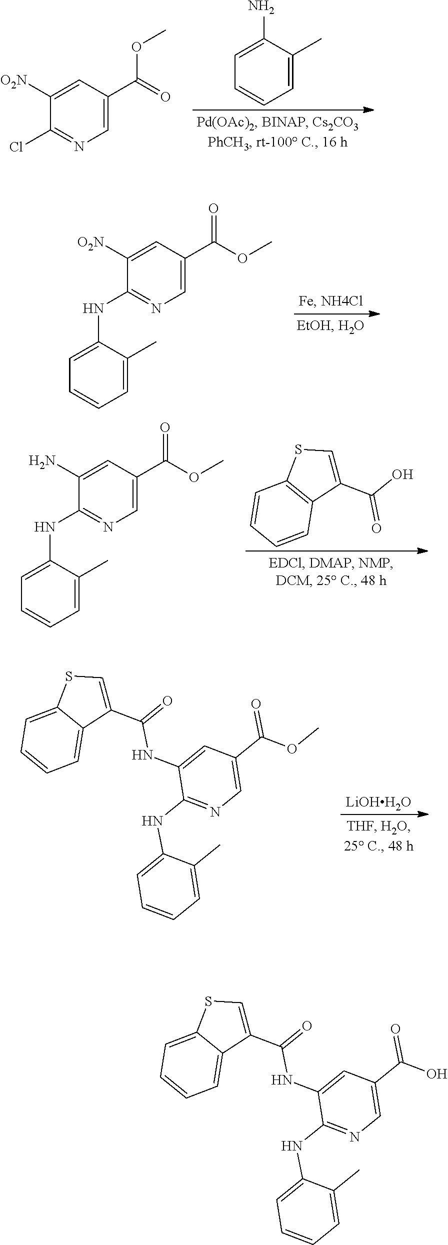

### Step 1. methyl 5-nitro-6-(o-tolylamino)nicotinate

A solution of methyl 6-chloro-5-nitronicotinate (1.5 g, 7 mmol), o-toluidine (3 g, 7 mmol), cesium carbonate (7 g, 21 mmol) in toluene (25 mL) was degassed with argon for 15 minutes at rt. Palladium (II) acetate (0.06 g, 0.26 mmol) and BINAP (0.18 g, 0.26 mmol) were then added to the reaction mixture, was again degassed with argon for 10 minutes and heated at 100° C. for 16 hours. The progress of the reaction was monitored by TLC and LCMS. The reaction mixture was passed through celite bed, and the bed was washed with ethyl acetate twice and the combined filtrate was concentrated under reduced pressure. The crude compound was purified by combi flash column chromatography (Silica gel) using ethyl acetate and hexane as eluent to afford methyl 5-nitro-6-(o-tolylamino)nicotinate (1.6 g, 80% yield). LC-MS m/z 288.10 [M+H]⁺.

### Step 2. methyl 5-amino-6-(o-tolylamino)nicotinate

To a solution of methyl 5-nitro-6-(o-tolylamino)nicotinate (3 g, 10.5 mmol) in ethanol (100 mL) were added iron (3 g, 60 mmol) and saturated ammonium chloride in water (40.0 mL). The solution was heated to 75° C. and stirred for 2 hours. The mixture was filtered through celite bed at hot condition, celite bed was washed with excess methanol in DCM (9:1). The combined filtrate was concentrated under reduced pressure to obtain the crude product, which was then purified by chromatography by neutral alumina column to obtain methyl 5-amino-6-(o-tolylamino)nicotinate (2.20 g, 82% yield). LC-MS m/z 258 [M+H]⁺.

### Step 3. methyl 5-(benzo[b]thiophene-3-carboxamido)-6-(o-tolylamino)nicotinate

Methyl 5-(benzo[b]thiophene-3-carboxamido)-6-(o-tolylamino)nicotinate was prepared from methyl 5-amino-6-(o-tolylamino)nicotinate (700 mg, 2.72 mmol) and benzo[b]thiophene-3-carboxylic acid (485 mg, 2.72 mmol) using acid amine coupling general procedure C. (727 mg, 64% yield). LC-MS m/z 418 [M+H]⁺

### Step 4. 5-(benzo[b]thiophene-3-carboxamido)-6-(o-tolylamino)nicotinic acid

5-(benzo[b]thiophene-3-carboxamido)-6-(o-tolylamino)nicotinic acid was prepared from methyl 5-(benzo[b]thiophene-3-carboxamido)-6-(o-tolylamino)nicotinate (100 mg, 0.24 mmol) using ester hydrolysis general procedure B (80 mg, 83% yield). LC-MS m/z 404.1 [M+H]⁺

### Preparation of 5-(benzo[b]thiophene-3-carboxamido)-6-((2-chloro-5-fluorophenyl)amino)nicotinic acid

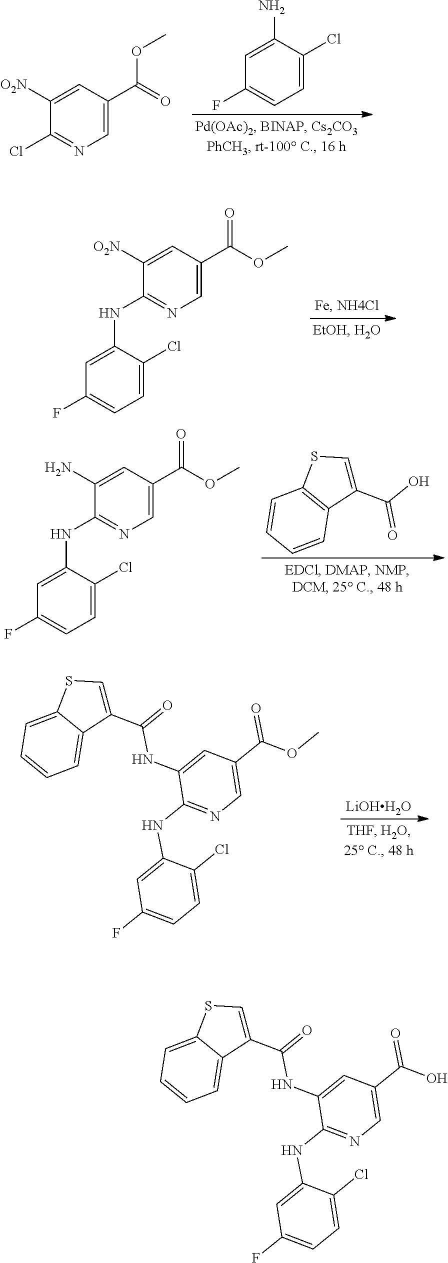

### Step 1. methyl 6-((2-chloro-5-fluorophenyl)amino)-5-nitronicotinate

A solution of methyl 6-chloro-5-nitronicotinate (3 g, 14 mmol), 2-chloro-5-fluoroaniline (2 g, 14 mmol), cesium carbonate (14 g, 42 mmol) in Toluene (50 mL) was degassed with argon for 15 minutes at rt. Palladium (II) acetate (0.12 g, 0.55 mmol) and BINAP (0.35 g, 0.55 mmol) were then added to the reaction mixture, was again degassed with argon for 10 minutes and heated at 100° C. for 16 hours. The progress of the reaction was monitored by TLC and LCMS. The reaction mixture was passed through celite bed, and the bed was washed with ethyl acetate twice and the combined filtrate was concentrated under reduced pressure. The crude compound was purified by combi flash column chromatography (Silica gel) using ethyl acetate and hexane as eluent to afford methyl 6-((2-chloro-5-fluorophenyl)amino)-5-nitronicotinate (3.50 g, 78% yield) as a yellow solid. LC-MS m/z 326.10 [M+H]⁺, Rt=0.94 min.

### Step 2. methyl 5-amino-6-((2-chloro-5-fluorophenyl)amino)nicotinate

To a solution of methyl 6-((2-chloro-5-fluorophenyl)amino)-5-nitronicotinate (4 g, 0.01 mol) in ethanol (160.0 mL) were added iron (3 g, 0.06 mol) and saturated ammonium chloride in water (40.0 mL). The solution was heated to 75° C. and stirred for 2 hours. The mixture was filtered through celite bed at hot condition, celite bed was washed with excess methanol in DCM (9:1). The combined filtrate was concentrated under reduced pressure to obtain the crude product, which was then purified by chromatography by neutral alumina column, Gradient Elusion, 15-40% ethyl acetate in hexane to obtain methyl 5-amino-6-((2-chloro-5-fluorophenyl)amino)nicotinate (2.80 g, 76% yield) as a yellowish solid. LC-MS m/z 296 [M+H]⁺, Rt=0.897 min, HPLC: 98.29%, Rt=5.28 min.

### Step 3. methyl 5-(benzo[b]thiophene-3-carboxamido)-6-((2-chloro-5-fluorophenyl)amino)nicotinate

Methyl 5-(benzo[b]thiophene-3-carboxamido)-6-((2-chloro-5-fluorophenyl)amino)nicotinate was prepared from methyl 5-amino-6-((2-chloro-5-fluorophenyl)amino)nicotinate (530 mg, 1.79 mmol) using acid amine coupling general procedure C and benzo[b]thiophene-3-carboxylic acid (319 mg, 1.79 mmol), (600 mg, 60% yield) as a grey solid. LC-MS m/z 456.0 [M+H]⁺, Rt=1.02 min, HPLC: 80.38%, Rt=6.16 min.

### Step 4. 5-(benzo[b]thiophene-3-carboxamido)-6-((2-chloro-5-fluorophenyl)amino)nicotinic acid

5-(benzo[b]thiophene-3-carboxamido)-6-((2-chloro-5-fluorophenyl)amino)nicotinic acid was prepared from methyl 5-(benzo[b]thiophene-3-carboxamido)-6-((2-chloro-5-fluorophenyl)amino)nicotinate (95 mg, 0.21 mmol) using ester hydrolysis general procedure B (75 mg, 81% yield) as a tan solid. LC-MS m/z 442.15 [M+H]⁺, Rt=0.89 min, HPLC: 97.23%, Rt=7.0 min. ¹H NMR (400 MHz, DMSO-D₆) δ=10.63-10.51 (m, 1H), 8.78 (s, 1H), 8.72-8.66 (m, 1H), 8.53-8.44 (m, 2H), 8.23 (d, J=1.9 Hz, 1H), 8.17-8.07 (m, 2H), 7.56-7.42 (m, 3H), 6.96-6.86 (m, 1H) ppm.

### Preparation of 1-(2-chloro-5-fluorophenyl)-7-(3-fluoro-5-(trifluoromethyl)benzamido)-3-oxoisoindoline-5-carboxylic acid

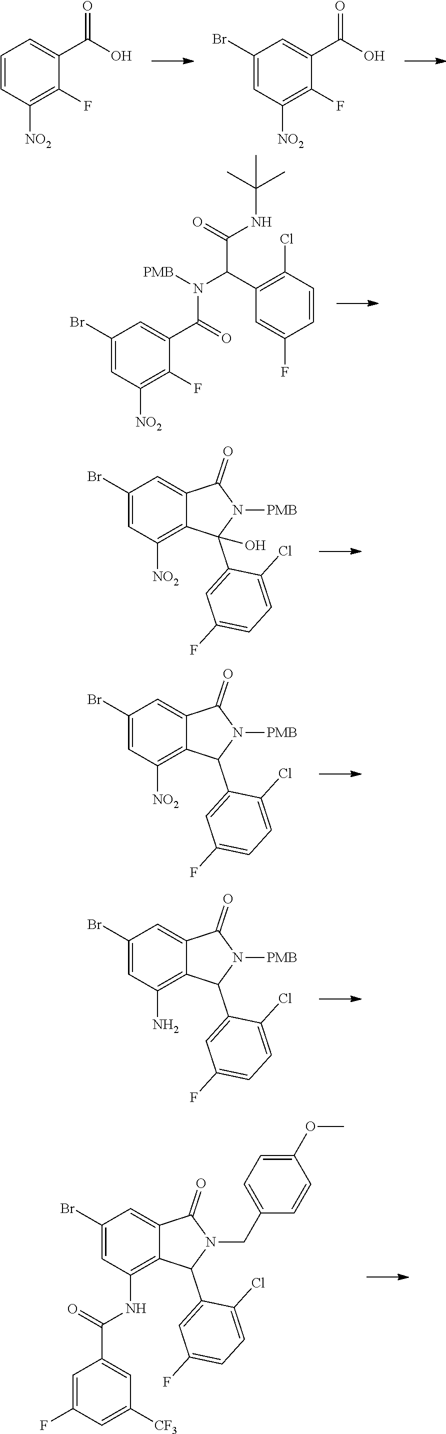

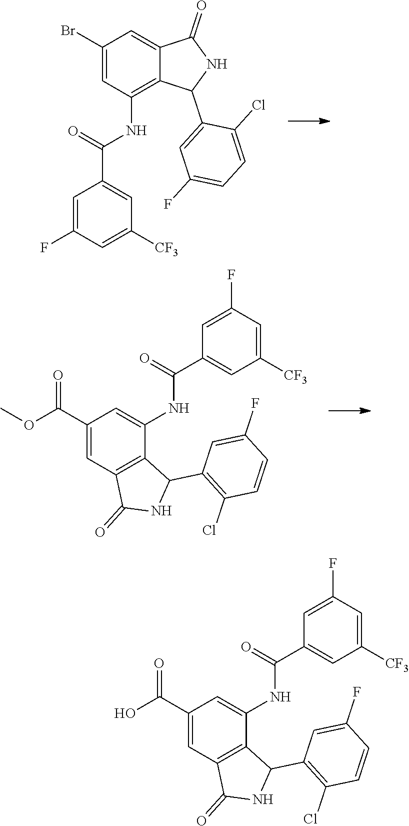 [SMILES: O=C(Nc1cc(Br)cc2c1C(c1cc(F)ccc1Cl)NC2=O)c1cc(F)cc(C(F)(F)F)c1 ; COC(=O)c1cc(NC(=O)c2cc(F)cc(C(F)(F)F)c2)c2c(c1)C(=O)NC2c1cc(F)ccc1Cl ; O=C(O)c1cc(NC(=O)c2cc(F)cc(C(F)(F)F)c2)c2c(c1)C(=O)NC2c1cc(F)ccc1Cl]

### Step 1. 5-bromo-2-fluoro-3-nitrobenzoic acid

To a solution of 2-fluoro-3-nitrobenzoic acid (185 g, 999 mmol) in H₂SO₄ (1.20 L) was added 1,3-dibromo-5,5-dimethylhydantoin (143 g, 500 mmol) at 20° C., the mixture was stirred at 85° C. for 2 h. The mixture was cooled to 20° C., which was then poured to ice water (5.00 L) and filtered. The filtered cake was concentrated under vacuum to afford 5-bromo-2-fluoro-3-nitrobenzoic acid (240 g, 84.5% yield) as a yellow solid. ¹H NMR (400 MHz, DMSO-d₆) δ 14.15 (br, 1H), 8.55 (d, J=3.2 Hz, 1H), 8.32-8.29 (m, 1H).

### Step 2. 5-bromo-N-(2-(tert-butylamino)-1-(2-chloro-5-fluorophenyl)-2-oxoethyl)-2-fluoro-N-(4-methoxybenzyl)-3-nitrobenzamide

To a solution of 2-chloro-5-fluorobenzaldehyde (67.0 g, 423 mmol) in MeOH (1.20 L) was sequentially added the following reagents, (4-methoxyphenyl)methanamine (58.0 g, 423 mmol), 5-bromo-2-fluoro-3-nitrobenzoic acid (120 g, 423 mmol), and tert-butyl isocyanide (35.1 g, 423 mmol) at 20° C. under N₂. The mixture was stirred at 20° C. for 2 h. The mixture was directly concentrated. The residue was purified by column chromatography (SiO₂, Petroleum ether/Ethyl acetate=50:1 to 5:1, R_{f}=0.40) to afford 5-bromo-N-(2-(tert-butylamino)-1-(2-chloro-5-fluorophenyl)-2-oxoethyl)-2-fluoro-N-(4-methoxybenzyl)-3-nitrobenzamide (248 g, 88.6% yield) as a yellow solid. MS: m/z=648.1 [M+Na]⁺; ¹H NMR (400 MHz, DMSO-d₆) δ 8.52 (br, 1H), 8.26-8.20 (m, 1H), 7.90 (s, 1H), 7.61-7.51 (m, 1H), 7.28-7.08 (m, 3H), 6.89-6.77 (m, 2H), 6.62-6.60 (m, 2H), 5.39-5.13 (m, 1H), 4.55-4.27 (m, 1H), 3.65 (s, 3H), 1.30-1.25 (m, 9H).

### Step 3. 6-bromo-3-(2-chloro-5-fluorophenyl)-3-hydroxy-2-(4-methoxybenzyl)-4-nitroisoindolin-1-one

To a solution of 5-bromo-N-(2-(tert-butylamino)-1-(2-chloro-5-fluorophenyl)-2-oxoethyl)-2-fluoro-N-(4-methoxybenzyl)-3-nitrobenzamide (243 g, 367 mmol) in ACN (1.50 L) was added 2-tert-butyl-1,1,3,3-tetramethylguanidine (94.3 g, 550 mmol) at 20° C. The mixture was stirred at 50° C. for 2 h. The mixture was concentrated to give 6-bromo-3-(2-chloro-5-fluorophenyl)-3-hydroxy-2-(4-methoxybenzyl)-4-nitroisoindolin-1-one (191 g, crude) as a black oil. MS: m/z=522.9 [M+H]⁺.

### Step 4. 6-bromo-3-(2-chloro-5-fluorophenyl)-2-(4-methoxybenzyl)-4-nitroisoindolin-1-one

To a solution of 6-bromo-3-(2-chloro-5-fluorophenyl)-3-hydroxy-2-(4-methoxybenzyl)-4-nitroisoindolin-1-one (191 g, 367 mmol) in TFA (1.50 L) was added triethylsilane (213 g, 1.83 mol) at 20° C. The mixture was stirred at 90° C. for 5 h. The mixture was directly concentrated to give 6-bromo-3-(2-chloro-5-fluorophenyl)-2-(4-methoxybenzyl)-4-nitroisoindolin-1-one (186 g, crude) as a green oil. MS: m/z=505.9 [M+H]⁺.

### Step 5. 4-amino-6-bromo-3-(2-chloro-5-fluorophenyl)-2-(4-methoxybenzyl)isoindolin-1-one

To a solution of 6-bromo-3-(2-chloro-5-fluorophenyl)-2-(4-methoxybenzyl)-4-nitroisoindolin-1-one (186 g, 367 mmol) in EtOH (1.20 L) and AcOH (150 mL) was added NH₄Cl (137 g, 2.57 mol) at 20° C. The mixture was heated to 50° C. and added Fe (143 g, 2.57 mol) in portions. The mixture was stirred at 80° C. for 1 h. The mixture was diluted with MeOH (2.00 L) and ethyl acetate (2.00 L). Then the mixture was poured into water (4.00 L). The mixture was extracted with ethyl acetate (2.00 L×3). The combined organic layers were washed with brine (2.50 L), dried over Na₂SO₄, filtered, and concentrated. The crude product was purified by silica gel column chromatography (Petroleum ether/Ethyl acetate=50/1 to 5/1, R_{f}=0.40). Then the crude product was purified by prep-HPLC (column: Phenomenex luna C18 250, 50 mm, 10 um; mobile phase: [water (0.225% FA)-ACN]; B %: 40%-70%, 30 min) to afford 4-amino-6-bromo-3-(2-chloro-5-fluorophenyl)-2-(4-methoxybenzyl)isoindolin-1-one (30.5 g, 14.2% yield) as an off-white solid. MS: m/z=476.8 [M+H]⁺; ¹H NMR (400 MHz, CDCl₃) δ 7.49 (d, J=1.6 Hz, 1H), 7.45-7.41 (m, 1H), 7.12-7.08 (m, 2H), 7.05-7.00 (m, 1H), 6.88 (d, J=1.6 Hz, 1H), 6.83-6.79 (m, 2H), 6.52 (dd, J=3.2, 3.2 Hz, 1H), 5.70 (d, J=1.6 Hz, 1H), 5.17 (d, J=14.8 Hz, 1H), 3.79 (s, 3H), 3.68 (d, J=14.8 Hz, 1H).

### Step 6: N-[6-bromo-3-(2-chloro-5-fluorophenyl)-2-[(4-methoxyphenyl)methyl]-1-oxo-2,3-dihydro-1H-isoindol-4-yl]-3-fluoro-5-(trifluoromethyl)benzamide

4-Amino-6-bromo-3-(2-chloro-5-fluorophenyl)-2-[(4-methoxyphenyl)methyl]-2,3-dihydro-1H-isoindol-1-one (7.0 g, 14.7 mmol) was dissolved in 500 mL of ACN and treated with pyridine (2.32 g, 29.4 mmol) and 3-fluoro-5-(trifluoromethyl)benzoyl chloride (3.33 g, 14.7 mmol). After an hour, a second aliquot of 3-fluoro-5-(trifluoromethyl)benzoyl chloride (0.833 g, 3.67 mmol) was added and the reaction was allowed to stir for 72 h. The mixture was diluted with EtOAc and water. The organic layer was washed with brine, dried over sodium sulfate, filtered, then concentrated to yield N-[6-bromo-3-(2-chloro-5-fluorophenyl)-2-[(4-methoxyphenyl)methyl]-1-oxo-2,3-dihydro-1H-isoindol-4-yl]-3-fluoro-5-(trifluoromethyl)benzamide (11 g, 16.5 mmol)

### Step 7. N-[6-bromo-3-(2-chloro-5-fluorophenyl)-1-oxo-2,3-dihydro-1H-isoindol-4-yl]-3-fluoro-5-(trifluoromethyl)benzamide

N-[6-bromo-3-(2-chloro-5-fluorophenyl)-2-[(4-methoxyphenyl)methyl]-1-oxo-2,3-dihydro-1H-isoindol-4-yl]-3-fluoro-5-(trifluoromethyl)benzamide (8.5 g, 12.7 mmol) was added to a flask and dissolved in methane sulfonic acid (35 mL) and heated to 60° C. for 16 h.

Aqueous sodium carbonate (10%) was added to the mixture. The aqueous layer was extracted with EtOAc, the organics were then washed with brine, dried over sodium sulfate, filtered, and concentrated to afford N-[6-bromo-3-(2-chloro-5-fluorophenyl)-1-oxo-2,3-dihydro-1H-isoindol-4-yl]-3-fluoro-5-(trifluoromethyl)benzamide (6 g, 86.5% yield).

### Step 8. Methyl 1-(2-chloro-5-fluorophenyl)-7-(3-fluoro-5-(trifluoromethyl)benzamido)-3-oxoisoindoline-5-carboxylate

To a solution of N-(6-bromo-3-(2-chloro-5-fluorophenyl)-1-oxoisoindolin-4-yl)-3-fluoro-5-(trifluoromethyl)benzamide (120 g, 220 mmol) in MeOH (1.00 L) was added Et₃N (44.5 g, 440 mmol, 61.2 mL) and Pd(dppf)Cl₂ (16.1 g, 22.0 mmol) at 25° C. The mixture was degassed and purged with CO₂ three times. The mixture was heated to 80° C. and stirred at 80° C. for 16 hrs under CO (50 psi). The mixture was concentrated, and the residue was purified by column chromatography (SiO₂, petroleum ether:ethyl acetate=50:1-0:1, R_{f}=0.1) to give methyl 1-(2-chloro-5-fluorophenyl)-7-(3-fluoro-5-(trifluoromethyl)benzamido)-3-oxoisoindoline-5-carboxylate (58.0 g, 103 mmol, 47.0% yield, 93.6% purity) as a pink solid.

### Step 9. 1-(2-chloro-5-fluorophenyl)-7-(3-fluoro-5-(trifluoromethyl)benzamido)-3-oxoisoindoline-5-carboxylic acid

Methyl 1-(2-chloro-5-fluorophenyl)-7-(3-fluoro-5-(trifluoromethyl)benzamido)-3-oxoisoindoline-5-carboxylate was subjected to ester hydrolysis general procedure A to give crude 1-(2-chloro-5-fluorophenyl)-7-(3-fluoro-5-(trifluoromethyl)benzamido)-3-oxoisoindoline-5-carboxylic acid, which was used directly in subsequent reactions.

### Preparation of tert-butyl (R)-2-((1-(6-methyl-4-oxo-2-(piperazin-1-yl)-4H-chromen-8-yl)ethyl)amino)benzoate and tert-butyl (S)-2-((1-(6-methyl-4-oxo-2-(piperazin-1-yl)-4H-chromen-8-yl)ethyl)amino)benzoate

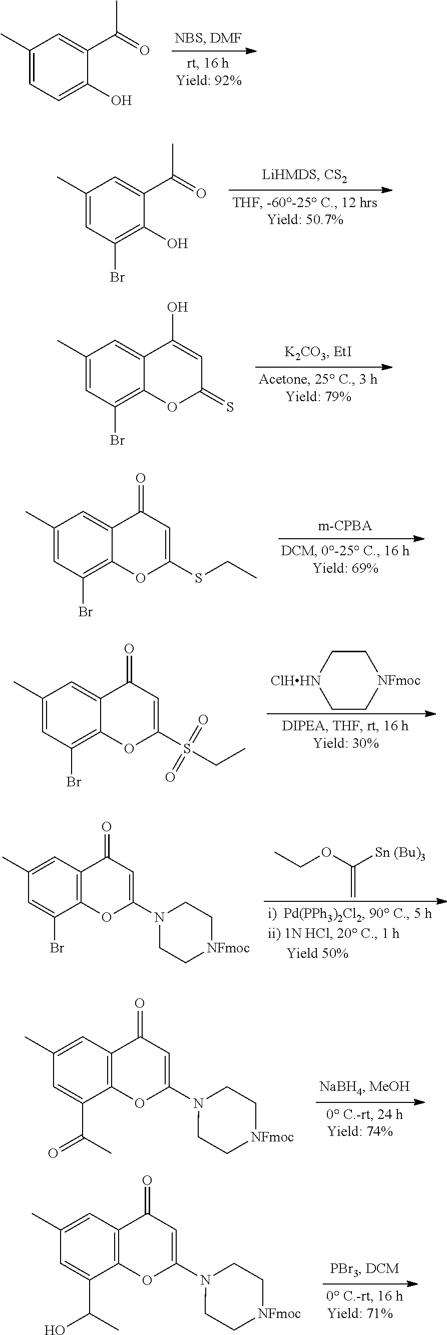

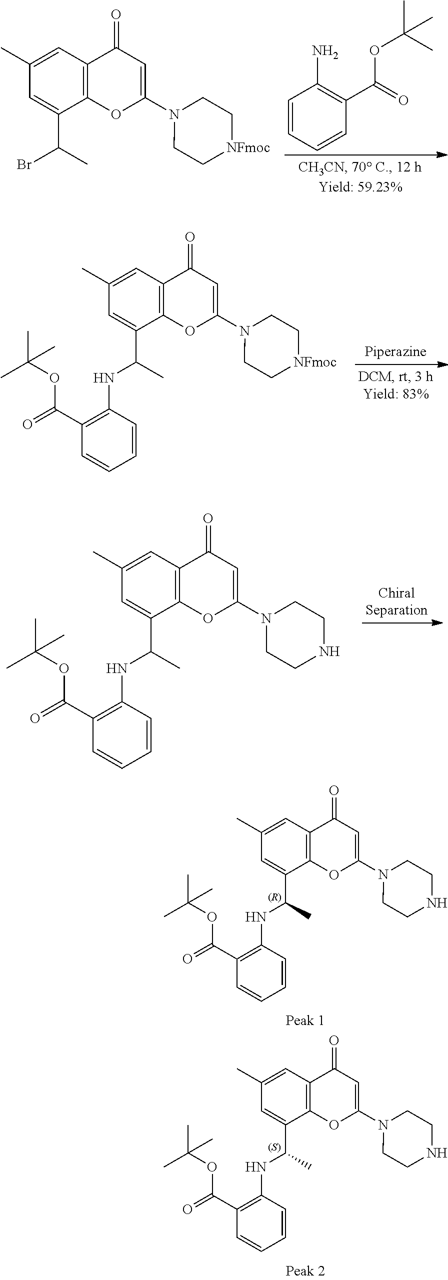

### Step 1. 1-(3-bromo-2-hydroxy-5-methylphenyl)ethan-1-one

N-Bromo succinimide (18 g, 0.10 mol) in DMF (75 mL) was added to a solution of 1-(2-hydroxy-5-methylphenyl)ethan-1-one (15 g, 0.10 mol) in DMF (75 mL) at 0° C. in a dropwise manner. The reaction mixture was stirred for 16 h at RT. After completion of the reaction as monitored by TLC and LCMS analysis, the solvents were removed under reduced pressure and the resulting residue was diluted with water (300 mL) and stirred for 2 h, solid separated out was collected by vacuum filtration to provide 1-(3-bromo-2-hydroxy-5-methylphenyl)ethan-1-one (21 g, 92% yield) as an off white solid. LC-MS m/z 228.95 [M+2H]⁺, Rt=0.86 min.

### Step 2. 8-bromo-4-hydroxy-6-methyl-2H-chromene-2-thione

To a solution of 1-(3-bromo-2-hydroxy-5-methylphenyl)ethan-1-one (21.0 g, 92.0 mmol) in THF (100 mL) was cooled to −60° C. Then LiHMDS (366.8 mL, 366.8 mmol) was added to the mixture in a drop wise manner at −60° C. The mixture was stirred for 2 h slowly and warmed up to −10° C. The mixture was cooled to −20° C., CS₂ (11.0 mL, 183 mmol) was added to the mixture at −20° C. The mixture was warmed up to 25° C. slowly and stirred at 25° C. for 12 hrs. The mixture was poured into ice water (250 mL) and citric acid was added to adjust pH=4. The mixture was stirred for 2 h at 25° C. until H₂S stopped to generate. The mixture was extracted with DCM (300 mL×2), dried over Na₂SO₄, filtered and the filtrate was concentrated under vacuum. The crude product was triturated with EtOAc/hexanes (1/1, 100 mL) provided 8-bromo-4-hydroxy-6-methyl-2H-chromene-2-thione (14.5 g, 50.7% yield) as a yellow solid. LC-MS m/z 272.55 [M+2H]⁺, Rt=1.70 min, HPLC: 86.87%, Rt=6.21 min.

### Step 3. 8-bromo-2-(ethylthio)-6-methyl-4H-chromen-4-one

To a solution of 8-bromo-4-hydroxy-6-methyl-2H-chromene-2-thione (7.0 g, 26 mmol) in acetone (100 mL) was added EtI (4.4 g, 28 mmol) and K₂CO₃ (3.6 g, 26 mmol) at 25° C. The mixture was stirred at 25° C. for 3 h. After completion of the reaction as monitored by TLC and LCMS analysis, the reaction mixture concentrated under reduced pressure. The resulting residue was dissolved in H₂O (200 mL). The aqueous phase was extracted with DCM (400 mL×2). The combined organic phase was washed with brine (100 mL), dried over anhydrous Na₂SO₄, filtered, and concentrated under reduced pressure. The crude product was triturated with pentane (70 mL) to give 8-bromo-2-(ethylthio)-6-methyl-4H-chromen-4-one (6.1 g, 79% yield) as a yellow solid. LC-MS m/z 300.60 [M+2H]⁺, Rt=1.62 min, HPLC: 99.62%, Rt=7.69 min.

### Step 4. 8-bromo-2-(ethylsulfonyl)-6-methyl-4H-chromen-4-one

To a solution of 8-bromo-2-(ethylthio)-6-methyl-4H-chromen-4-one (6.0 g, 20 mmol) in DCM (80 mL) was added m-CPBA (8.7 g, 50 mmol) at 0° C. The mixture was stirred at 25° C. for 16 h. After completion of the reaction as monitored by TLC and LCMS analysis, the reaction mixture was poured into H₂O (200 mL). The mixture was filtered to give the filtrate. The aqueous phase was extracted with DCM (400 mL×2). The combined organic phase was washed with saturated sodium sulfite solutions (150 mL×3) and brine (100 mL×2), dried with anhydrous Na₂SO₄, filtered, and concentrated under reduced pressure to give 8-bromo-2-(ethylsulfonyl)-6-methyl-4H-chromen-4-one (5 g, 69% yield) as a yellow solid. LC-MS m/z 330.60 [M+H]⁺, Rt=1.51 min, HPLC: 91.54%, Rt=4.41 min.

### Step 5. (9H-fluoren-9-yl)methyl 4-(8-bromo-6-methyl-4-oxo-4H-chromen-2-yl)piperazine-1-carboxylate

To a solution of 8-bromo-2-(ethylsulfonyl)-6-methyl-4H-chromen-4-one (3.0 g, 9 mmol) in THF (60 mL) was added 9H-fluoren-9-yl)methyl piperazine-1-carboxylate hydrochloride (2 g, 7 mmol), followed by DIPEA (2 mL, 0.01 mol) at 25° C. The mixture was stirred at 25° C. for 16 h. After completion of the reaction as monitored by TLC and LCMS analysis, the reaction mixture was concentrated under reduced pressure. The resulting residue was diluted with saturated ammonium chloride (30 mL) and extracted with DCM (200 mL×2). The combined organic layers were washed with brine (10 volume) and was dried over Na₂SO₄ and the solvents were removed under reduced pressure. The crude compound thus obtained. was triturated with EtOAc/Hexane (1:5, 50.0 mL) followed by diethyl ether to get the (9H-fluoren-9-yl)methyl 4-(8-bromo-6-methyl-4-oxo-4H-chromen-2-yl)piperazine-1-carboxylate (1.60 g, 30% yield) as an off-white solid. LC-MS m/z 547.05 [M+2H]⁺, Rt=1.02 min, HPLC: 95.08%, Rt=8.28 min.

### Step 6. (9H-fluoren-9-yl)methyl 4-(8-acetyl-6-methyl-4-oxo-4H-chromen-2-yl)piperazine-1-carboxylate

A solution of (9H-fluoren-9-yl) methyl 4-(8-bromo-6-methyl-4-oxo-4H-chromen-2-yl) piperazine-1-carboxylate (0.8 g, 1.0 mmol) in 1,4-Dioxane (8.0 mL) was bubbled with argon for 10 minutes and then tributyl(1-ethoxyvinyl) stannane (0.8 g, 2.0 mmol) and Pd(PPh₃)₂Cl₂ (0.1 g, 0.1 mmol) was added and bubbled with argon for 20 minutes. Later it was stirred at 90° C. for 5 hours. After completion of the reaction as monitored by TLC and LCMS analysis, quenched with 1N HCl at 20° C. and stirred for 1 hour. LCMS showed intermediate state was consumed completely and desired mass was detected. Further it was filtered through celite bed, and the filtrate was poured into H₂O (10 mL). The aqueous phase was extracted with DCM (100 mL×2). The combined organic phase was washed with brine (10 mL), dried with anhydrous Na₂SO₄, filtered, and the solvents were removed under reduced pressure. The crude compound thus obtained was purified by chromatography using a Redi Sep 12 g SiO₂ column. It was eluted with a gradient of 3-5% of MeOH in DCM to obtain (9H-fluoren-9-yl)methyl 4-(8-acetyl-6-methyl-4-oxo-4H-chromen-2-yl)piperazine-1-carboxylate (400 mg, 50% yield) as an off-white solid. LC-MS m/z 509.15 [M+H]⁺, Rt=0.93 min, HPLC: 85.13%, Rt=13.19 min.

### Step 7. (9H-fluoren-9-yl)methyl 4-(8-(1-hydroxyethyl)-6-methyl-4-oxo-4H-chromen-2-yl)piperazine-1-carboxylate

To a stirred solution of (9H-fluoren-9-yl)methyl 4-(8-acetyl-6-methyl-4-oxo-4H-chromen-2-yl)piperazine-1-carboxylate (3.0 g, 5.9 mmol) dissolved in methanol (30.0 mL) was added NaBH4 (0.89 g, 24 mmol) lot wise manner at 0° C. After completion of addition, reaction mass was allowed to rt and stirred for 24 h at rt. After completion of the reaction as monitored by TLC and LCMS analysis, the reaction mixture was concentrated under reduced pressure. The residue was diluted with saturated ammonium chloride (30 mL) and extracted with DCM (200 mL×2) times. The combined organic layers were washed with brine (20 mL) and was dried over Na₂SO₄ and the solvents were removed under reduced pressure. The crude compound thus obtained was triturated with EtOAc/Hexane (1:5, 50.0 mL) to get the (9H-fluoren-9-yl)methyl 4-(8-(1-hydroxyethyl)-6-methyl-4-oxo-4H-chromen-2-yl)piperazine-1-carboxylate (2.50 g, 74% yield) as a pale-yellow solid. LC-MS m/z 511.00 [M+H]⁺, Rt=1.50 min, HPLC: 88.66%, Rt=4.28 min.

### Step 8. (9H-fluoren-9-yl)methyl 4-(8-(1-bromoethyl)-6-methyl-4-oxo-4H-chromen-2-yl)piperazine-1-carboxylate

To a stirred solution of (9H-fluoren-9-yl)methyl 4-(8-(1-hydroxyethyl)-6-methyl-4-oxo-4H-chromen-2-yl)piperazine-1-carboxylate (2.5 g, 4.90 mmol) dissolved in DCM (30 mL) was added PBr₃ (1.6 g, 5.9 mmol) drop wise at 0° C. The resulting reaction mass was allowed to rt and stirred for 16 h at rt. After completion of the reaction as monitored by TLC and LCMS analysis, the reaction mixture was concentrated under reduced pressure. The residue was diluted with water (150 mL), basified with sat. NaHCO₃ solution (30.0 mL, p^{H}=8) and extracted with DCM (100 mL×2). The combined organic layers were washed with brine (20 mL) and was dried over Na₂SO₄ and the solvents were removed under reduced to get the (9H-fluoren-9-yl)methyl 4-(8-(1-bromoethyl)-6-methyl-4-oxo-4H-chromen-2-yl)piperazine-1-carboxylate (2 g, 71% yield) as an off-white solid. LC-MS m/z 573.15 [M+H]⁺, Rt=1.65 min.

### Step 9. (9H-fluoren-9-yl)methyl 4-(8-(1-((2-(tert-butoxycarbonyl)phenyl)amino)ethyl)-6-methyl-4-oxo-4H-chromen-2-yl)piperazine-1-carboxylate

To a solution of (9H-fluoren-9-yl)methyl 4-(8-(1-bromoethyl)-6-methyl-4-oxo-4H-chromen-2-yl)piperazine-1-carboxylate (1.20 g, 2.10 mmol) in Acetonitrile (12 mL) was added tert-butyl 2-aminobenzoate (0.81 g, 4.2 mmol) at 25° C. The mixture was stirred at 70° C. for 12 h. After completion of the reaction as monitored by TLC and LCMS analysis, the reaction mixture was cooled to rt, filtered the undissolved materials and the filtrate was concentrated under reduced pressure to get the crude material, which was triturated with pentane (25 mL) followed by diethyl ether (25 mL) to get the (9H-fluoren-9-yl)methyl 4-(8-(1-((2-(tert-butoxycarbonyl)phenyl)amino)ethyl)-6-methyl-4-oxo-4H-chromen-2-yl)piperazine-1-carboxylate (1.70 g, 59.23% yield) as an off white solid. LC-MS m/z 686.30 [M+H]⁺.

### Step 10. tert-butyl 2-((1-(6-methyl-4-oxo-2-(piperazin-1-yl)-4H-chromen-8-yl)ethyl)amino)benzoate

To a solution of (9H-fluoren-9-yl)methyl 4-(8-(1-((2-(tert-butoxycarbonyl)phenyl)amino)ethyl)-6-methyl-4-oxo-4H-chromen-2-yl)piperazine-1-carboxylate (1.60 g, 2.30 mmol) in DCM (16 mL) was added piperazine (1.6 g, 19 mmol) at 0° C. and stirred for 3 hour at rt. After completion of the reaction as monitored by TLC and LCMS analysis, the reaction mixture was concentrated under reduced pressure. The resulting residue was quenched with ice cooled water and stirred for 3 h, filtered the resulting solid. The solid material was purified by column chromatography using a Redi Sep 12 g neutral alumina packed column. It was eluted with a gradient of 1-2% of MeOH in DCM to obtain the required compound 10 (900 mg, 83%, RLN-NB-2155-073-P1) as a white filmy solid.

### Step 11. tert-butyl (R)-2-((1-(6-methyl-4-oxo-2-(piperazin-1-yl)-4H-chromen-8-yl)ethyl)amino)benzoate

Further this material was chiral separated by chiral purification (Method F) to provide tert-butyl (R)-2-((1-(6-methyl-4-oxo-2-(piperazin-1-yl)-4H-chromen-8-yl)ethyl)amino)benzoate (380 mg, Peak 1) as a white filmy solid, LC-MS m/z 464.25 [M+H]⁺, Rt=1.36 min, Chiral HPLC: 97.36%, Rt=11.00 min.

### Step 11. tert-butyl (S)-2-((1-(6-methyl-4-oxo-2-(piperazin-1-yl)-4H-chromen-8-yl)ethyl)amino)benzoate

Further this material was chiral separated by chiral purification (Method F) to provide tert-butyl (S)-2-((1-(6-methyl-4-oxo-2-(piperazin-1-yl)-4H-chromen-8-yl)ethyl)amino)benzoate (340 mg, Peak 2) as a white filmy solid, LC-MS m/z 464.25 [M+H]⁺, Rt=1.36 min, Chiral HPLC: 98.60%, Rt=12.74 min.

### Preparation of 1-(2-chloro-5-fluorophenyl)-8-nitro-3-oxo-1,2,3,4-tetrahydropyrrolo[1,2-a]pyrazine-6-carboxylic acid

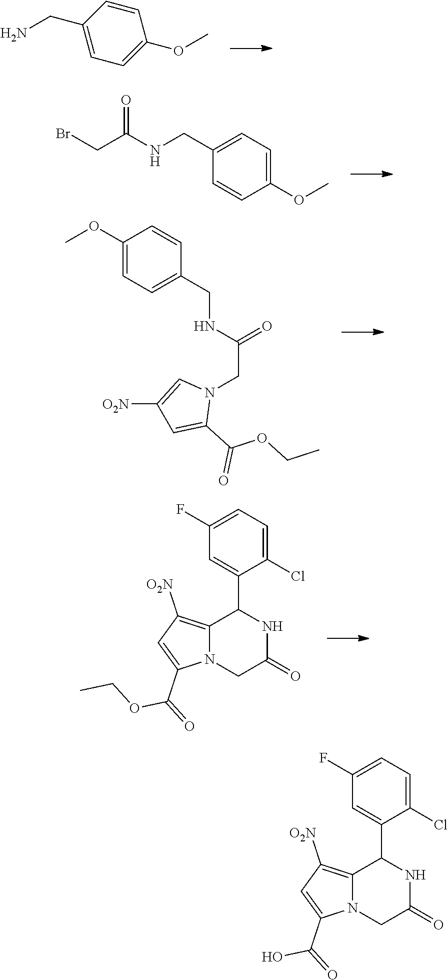

### Step 1. 2-bromo-N-(4-methoxybenzyl)acetamide

A round bottom flask was charged with 1-(4-methoxyphenyl)methanamine (12 g), triethylamine (8.84 g) and a stir bar. Tetrahydrofuran (120 mL) was added, and the solution was stirred at −70° C. 2-Bromoacetyl chloride (13.7 g) was added slowly, and the solution was stirred at room temperature overnight. The reaction was quenched with water and extracted with ethyl acetate. The organic layer was washed with brine, dried over Na₂SO₄ and concentrated to give the desired product (20 g), which was used in the next step without purification. LCMS: RT 1.077 min, [M+H]⁺ 258.05.

### Step 2. ethyl 1-(2-((4-methoxybenzyl)amino)-2-oxoethyl)-4-nitro-1H-pyrrole-2-carboxylate

A round bottom flask was charged with ethyl 4-nitro-1H-pyrrole-2-carboxylate (10 g) dissolved in acetonitrile (150 mL) and a stir bar. 2-(tert-butyl)-1,1,3,3-tetramethylguanidine (9.30 g) was added and the solution was stirred at room temperature for 5 minutes. Then 2-bromo-N-[(4-methoxyphenyl)methyl]acetamide (21.0 g) was added and the solution was stirred at room temperature for 2 hours. The reaction was quenched with water and extracted with ethyl acetate. The organic layer was washed with brine, dried over Na₂SO₄ and concentrated. A precipitate formed and was collected by filtration to give the product (10 g) as an off-white solid. CMS: RT 1.065 min, [M+H]⁺ 362.05.

### Step 3. ethyl 1-(2-chloro-5-fluorophenyl)-8-nitro-3-oxo-1,2,3,4-tetrahydropyrrolo[1,2-a]pyrazine-6-carboxylate

A round bottom flask was charged with ethyl 1-({[(4-methoxyphenyl)methyl]carbamoyl}methyl)-4-nitro-1H-pyrrole-2-carboxylate (260 mg), 2-chloro-5-fluorobenzaldehyde (136 mg) and a stir bar. Eaton's reagent (6.5 mL) was added, and the solution was stirred at 80° C. for 50 minutes. The reaction mixture was diluted with ethyl acetate, cooled to −20° C., quenched with saturated NaHCO₃ solution and extracted with ethyl acetate. The organic phase was dried over Na₂SO₄ and concentrated. The residue was purified by prep-TLC (petroleum ether:ethyl acetate 1:1) to the desired product (130 mg) as a yellow solid. LCMS: RT 1.098 min, [M+H]⁺ 381.95.

### Step 4. 1-(2-chloro-5-fluorophenyl)-8-nitro-3-oxo-1,2,3,4-tetrahydropyrrolo[1,2-a]pyrazine-6-carboxylic acid

A round bottom flask was charged with ethyl 1-(2-chloro-5-fluorophenyl)-8-nitro-3-oxo-1,2,3,4-tetrahydropyrrolo[1,2-a]pyrazine-6-carboxylate (1.5 g), trimethylstannanol (3.52 g) and a stir bar. Dichloroethane (7.5 mL) was added, and the solution was stirred at 80° C. overnight. The solution was concentrated and purified using C18 flash chromatography with the following conditions (mobile phase A: water, mobile phase B: acetonitrile; flow rate: 60 mL/min; Gradient: 0% B to 100% B in 40 minutes; wavelength: 254/220 nm) to give the desired product (300 mg) as a brown solid. LCMS: RT 0.749 min, [M−H]⁻ 351.80.

### Example 1

### N-(9-(5-(benzo[b]thiophene-3-carboxamido)-6-(o-tolylamino)nicotinamido)nonyl)-2-(2,6-dioxopiperidin-3-yl)-1-oxoisoindoline-5-carboxamide

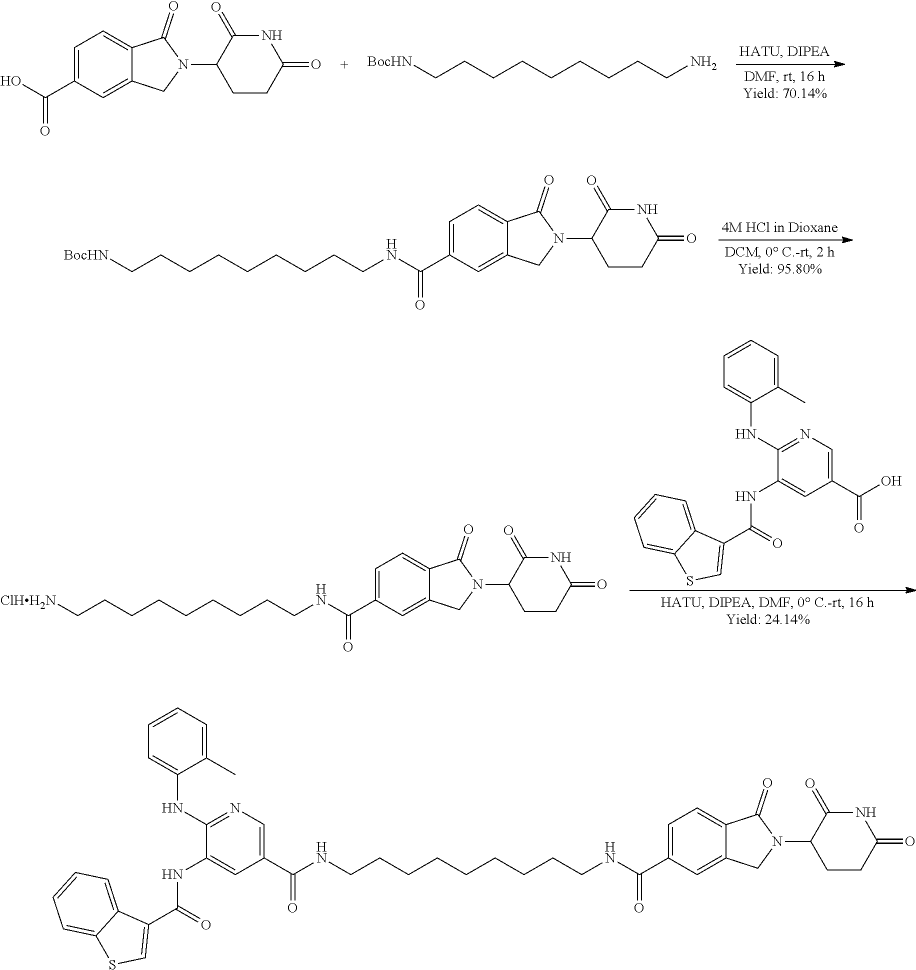

### Step 1. tert-butyl (9-(2-(2,6-dioxopiperidin-3-yl)-1-oxoisoindoline-5-carboxamido)nonyl)carbamate

To a solution of 2-(2,6-dioxopiperidine-3-yl)-1-oxoisoindoline-5-carboxylic acid (500 mg, 1.734 mmol) in DMF (5.0 mL) was added HATU (923.33 mg, 2.428 mmol) followed by DIPEA (0.9 mL, 5.203 mmol) at 0° C. After stirring for 30 minutes, Tert-butyl(9-aminononoyl) carbamate (582.69 mg, 2.254 mmol) was added at same temperature. The resulting reaction mixture was stirred at rt for 16 h and the reaction completion was monitored by TLC and LCMS. After completion, the reaction mixture was partitioned between 5% MeOH in DCM (50 mL) and ice-cold water (20 mL). Extraction was done twice with 5% MeOH in DCM (2×50 mL) and combined organic layer was dried over Na₂SO₄ and concentrated under reduced pressure to get the crude compound. The crude material was purified by column chromatography using a Redi Sep 12 g SiO₂ column. It was eluted with a gradient of 2-3% of MeOH in DCM to obtain tert-butyl (9-(2-(2,6-dioxopiperidin-3-yl)-1-oxoisoindoline-5-carboxamido)nonyl)carbamate (425 mg, 0.803 mmol, 46.36% yield) as a light brown solid. LCMS: product: Rt=1.474 min, m/z=529.35 (M+1) (99.36%).

### Step 2. N-(9-aminononyl)-2-(2,6-dioxopiperidin-3-yl)-1-oxoisoindoline-5-carboxamide hydrochloride

4M HCl in dioxane (2.0 mL) was added dropwise to a solution of tert-butyl (9-(2-(2,6-dioxopiperidin-3-yl)-1-oxoisoindoline-5-carboxamido)nonyl) carbamate (95 mg, 0.179 mmol) in DCM (1.0 mL) at 0° C. The resulting mixture was stirred for 2 hours at room temperature. After completion of the reaction, the reaction mixture was concentrated under reduced pressure and resulting residue was triturated with pentane, followed by diethyl ether to afford N-(9-aminononyl)-2-(2,6-dioxopiperidin-3-yl)-1-oxoisoindoline-5-carboxamide hydrochloride (80 mg, 0.172 mmol, 95.8%) as an off-white sticky solid. LCMS: product: Rt=1.252 min, m/z=429.20 (M+1) (98.96%) corresponds to free amine).

### Step 3. N-(9-(5-(benzo[b]thiophene-3-carboxamido)-6-(o-tolylamino)nicotinamido)nonyl)-2-(2,6-dioxopiperidin-3-yl)-1-oxoisoindoline-5-carboxamide

To a solution of 5-(benzo[b]thiophene-3-carboxamido)-6-(o-tolylamino) nicotinic acid (43 mg, 0.108 mmol) in DMF (1.0 mL) was added HATU (49 mg, 0.129 mmol) followed by DIPEA (56 mg, 0.430 mmol) at 0° C. After stirring for 30 minutes, N-(9-aminononyl)-2-(2,6-dioxopiperidin-3-yl)-1-oxoisoindoline-5-carboxamide hydrochloride (50 mg, 0.108 mmol) in DMF (0.5 mL) was added drop wise at same temperature. The resulting reaction mixture was stirred at rt for 16 h and the reaction completion was monitored by TLC and LCMS. After completion, the reaction mixture was partitioned between 5% MeOH in DCM (20 mL) and ice-cold water (10 mL). Extraction was done twice with 5% MeOH in DCM (2×20 mL) and combined organic layer was dried over Na₂SO₄ and concentrated under reduced pressure to get the crude compound. The crude was purified by preparative HPLC: Mobile Phase: A=0.10% HCOOH IN WATER B=ACN Column: LUNA Phenomenex (250 mm×21.2 mm), 5.0μ Flow: 18 ml/min, Gradient Program: (Time, B %): (0, 30) (2, 40) (8, 60), followed by the lyophilization of the purified sample to afford N-(9-(5-(benzo[b]thiophene-3-carboxamido)-6-(o-tolylamino)nicotinamido)nonyl)-2-(2,6-dioxopiperidin-3-yl)-1-oxoisoindoline-5-carboxamide (21 mg, 0.026 mmol, 24.14% yield, 96.81% purity) as a white solid. LCMS: product: Rt=0.853 min, m/z=[M+1]=814.50 (95.99%). HPLC: APSL-0098-013-F1, product: Rt=7.579 mins, 96.81% purity under 210 nm. ¹H NMR: (400 MHz, DMSO-d6) δ 11.00 (s, 1H), 10.05 (s, 1H), 8.70 (s, 1H), 8.61 (s, 1H), 8.51-8.47 (m, 1H), 8.45 (d, J=2.2 Hz, 1H), 8.34-8.28 (m, 1H), 8.22-8.14 (m, 1H), 8.13-8.07 (m, 2H), 8.03 (s, 1H), 7.98-7.93 (m, 1H), 7.79 (d, J=8.1 Hz, 1H), 7.53-7.42 (m, 3H), 7.24-7.14 (m, 2H), 7.08-7.02 (m, 1H), 5.17-5.09 (m, 1H), 4.55-4.33 (m, 2H), 3.25 (dd, J=5.8, 12.4 Hz, 4H), 2.91 (s, 1H), 2.65-2.56 (m, 2H), 2.44-2.38 (m, 1H), 2.17 (s, 3H), 2.06-1.98 (m, 1H), 1.52 (d, J=7.0 Hz, 4H), 1.30 (s, 9H).

### Example 2

### 5-(benzo[b]thiophene-3-carboxamido)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)-6-(o-tolylamino)nicotinamide

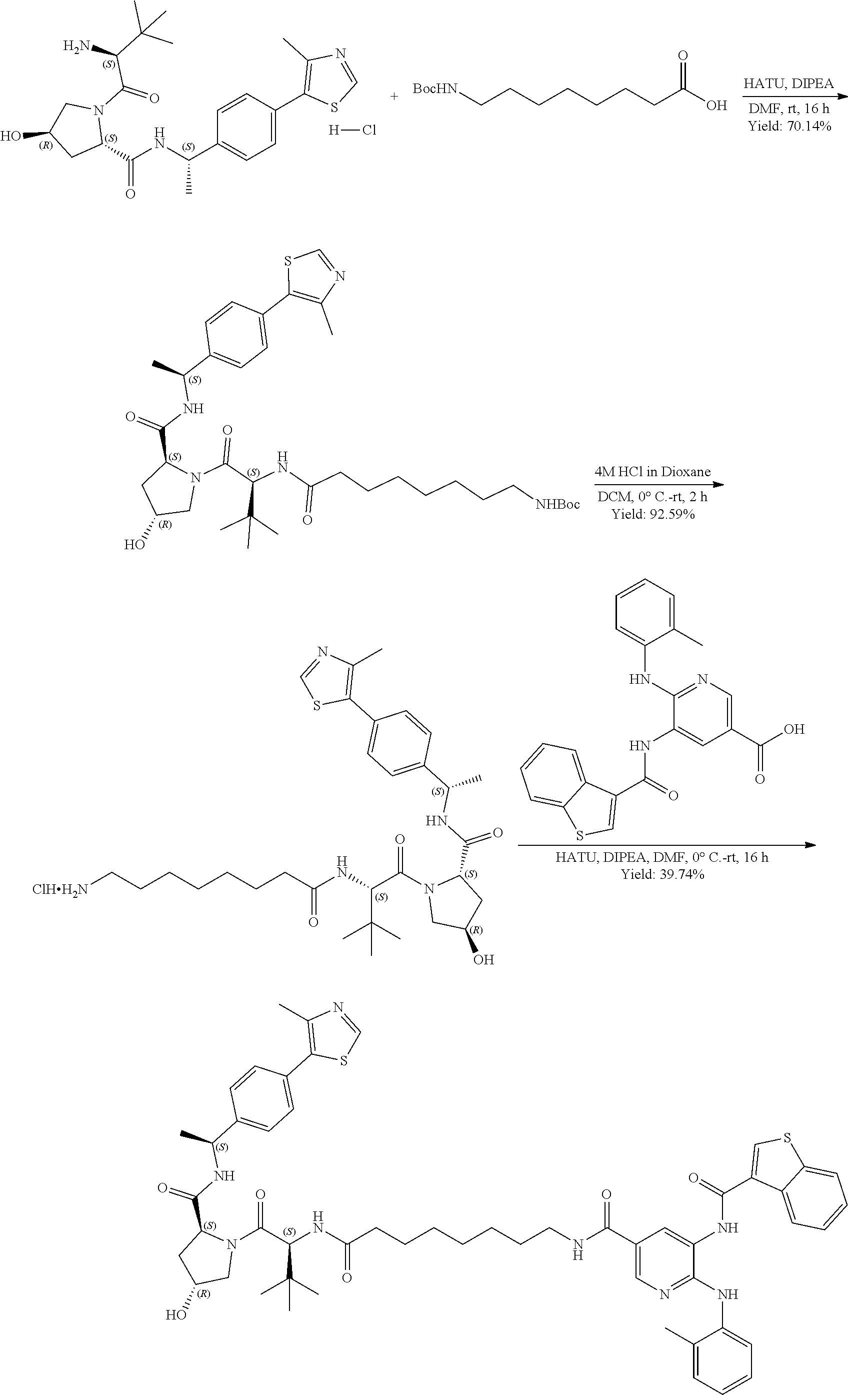

### Step 1. tert-butyl (8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamate

To a solution of 8-((tert-butoxycarbonyl) amino) octanoic acid (194.0 mg, 0.748 mmol) in DMF (3.0 mL) was added HA TU (308.26 mg, 0.810 mmol) followed by DIPEA (0.43 mL, 2.49 mmol) at 0° C. After stirring for 30 minutes, (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide hydrochloride. HCl (300 mg, 0.623 mmol) was added at same temperature. The resulting reaction mixture was stirred at rt for 16 h and the reaction completion was monitored by TLC and LCMS. After completion, the reaction mixture was partitioned between 5% MeOH in DCM (10 mL) and ice-cold water (10 mL). Extraction was done twice with 5% MeOH in DCM (2×20 mL) and combined organic layer was dried over Na₂SO₄ and concentrated under reduced pressure to get the crude compound. The crude material was purified by column chromatography using a Redi Sep 12 g SiO₂ column. It was eluted with a gradient of 2-3% of MeOH in DCM to obtain tert-butyl (8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamate (300 mg, 0.437 mmol, 70.14% yield) as a white sticky compound. LCMS: APSL-0087-020-P1, product: Rt=1.50 min, m/z=686.76 (M+1) (91.38%) corresponds to free amine). HPLC: APSL-0087-020-P1, product: Rt=6.32 mins, 98.04% purity under 210-400 nm. Chiral HPLC: product: Rt=10.33 mins, 92.67% purity under 210 nm.

### Step 2. (2S,4R)-1-((S)-2-(8-aminooctanamido)-3,3-dimethylbutanoyl)-4-hydroxy-N-((S)-1-(4-(4-methylthiazol-5-yl)phenyl)ethyl)pyrrolidine-2-carboxamide hydrochloride

4M HCl in dioxane (1.5 mL) was added dropwise to a solution of tert-butyl (8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamate (60 mg, 0.087 mmol) in DCM (1.0 mL) at 0° C. The resulting mixture was stirred for 2 hours at room temperature. After completion of the reaction, the reaction mixture was concentrated under reduced pressure and resulting residue was triturated with pentane, followed by diethyl ether to afford (2S,4R)-1-((S)-2-(8-aminooctanamido)-3,3-dimethylbutanoyl)-4-hydroxy-N-((S)-1-(4-(4-methylthiazol-5-yl)phenyl)ethyl)pyrrolidine-2-carboxamide hydrochloride (50 mg, 0.080 mmol, 92.59% yield) as an off-white sticky solid. LCMS: APSL-0098-016-C1, product: Rt=1.304 min, m/z=586.50 (M+1) (70.78%) corresponds to free amine).

### Step 3. 5-(benzo[b]thiophene-3-carboxamido)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)-6-(o-tolylamino)nicotinamide

To a solution of 5-(benzo[b]thiophene-3-carboxamido)-6-(o-tolylamino) nicotinic acid (32 mg, 0.080 mmol) in DMF (1.0 mL) was added HATU (37 mg, 0.096 mmol) followed by DIPEA (42 mg, 0.321 mmol) at 0° C. After stirring for 30 minutes, (2S,4R)-1-((S)-2-(8-aminooctanamido)-3,3-dimethylbutanoyl)-4-hydroxy-N-((S)-1-(4-(4-methylthiazol-5-yl)phenyl)ethyl)pyrrolidine-2-carboxamide hydrochloride (50 mg, 0.080 mmol) in DMF (0.5 mL) was added drop wise at same temperature. The resulting reaction mixture was stirred at rt for 16 h and the reaction completion was monitored by TLC and LCMS. After completion, the reaction mixture was partitioned between 5% MeOH in DCM (20 mL) and ice-cold water (10 mL). Extraction was done twice with 5% MeOH in DCM (2×20 mL) and combined organic layer was dried over Na₂SO₄ and concentrated under reduced pressure to get the crude compound. The crude was purified by preparative HPLC: Mobile Phase: A=0.1% HCOOH IN WATER B=ACN Column: XSELECT (C18, 19 mm×250 mm), Flow: 18 ml/min, Gradient Program: (Time, B %): (0, 25) (2, 30) (10, 60), followed by the lyophilization of the purified sample to afford 5-(benzo[b]thiophene-3-carboxamido)-N-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)-6-(o-tolylamino)nicotinamide (31 mg, 0.032 mmol, 39.74% yield) as a white solid. LCMS: APSL-0098-018-F1, product: Rt=0.907 min, m/z=[M−1]=969.40 (95.14%). HPLC: APSL-0098-018-F1, product: Rt=6.61 mins, 95.60% purity under 210-400 nm. Chiral HPLC: APSL-0098-018-F1, product: Rt=7.791 mins, 98.64% purity under 254 nm. ¹H NMR: APSL-0098-018-F1, (400 MHz, DMSO-d6) δ 10.05 (s, 1H), 8.98 (s, 1H), 8.72-8.69 (m, 1H), 8.49 (dd, J=1.2, 7.0 Hz, 1H), 8.45 (d, J=2.1 Hz, 1H), 8.39-8.30 (m, 2H), 8.18 (s, 1H), 8.13-8.07 (m, 2H), 7.78 (d, J=9.2 Hz, 1H), 7.48-7.41 (m, 5H), 7.40-7.36 (m, 2H), 7.24-7.17 (m, 2H), 7.08-7.02 (m, 1H), 5.11-5.06 (m, 1H), 4.91 (s, 1H), 4.51 (d, J=9.4 Hz, 1H), 4.42 (s, 1H), 4.31-4.24 (m, 1H), 3.60 (s, 2H), 3.24 (d, J=6.3 Hz, 2H), 2.45 (s, 3H), 2.23 (s, 1H), 2.18 (s, 3H), 2.12 (d, J=7.6 Hz, 1H), 2.05-1.96 (m, 1H), 1.78 (s, 1H), 1.53-1.46 (m, 4H), 1.37 (d, J=7.0 Hz, 3H), 1.28 (s, 6H), 0.92 (s, 9H).

### Example 3

### 5-(benzo[b]thiophene-3-carboxamido)-N-(9-((2-(2,6-dioxopiperidin-3-yl)-1,3-dioxoisoindolin-5-yl)amino)nonyl)-6-(o-tolylamino)nicotinamide

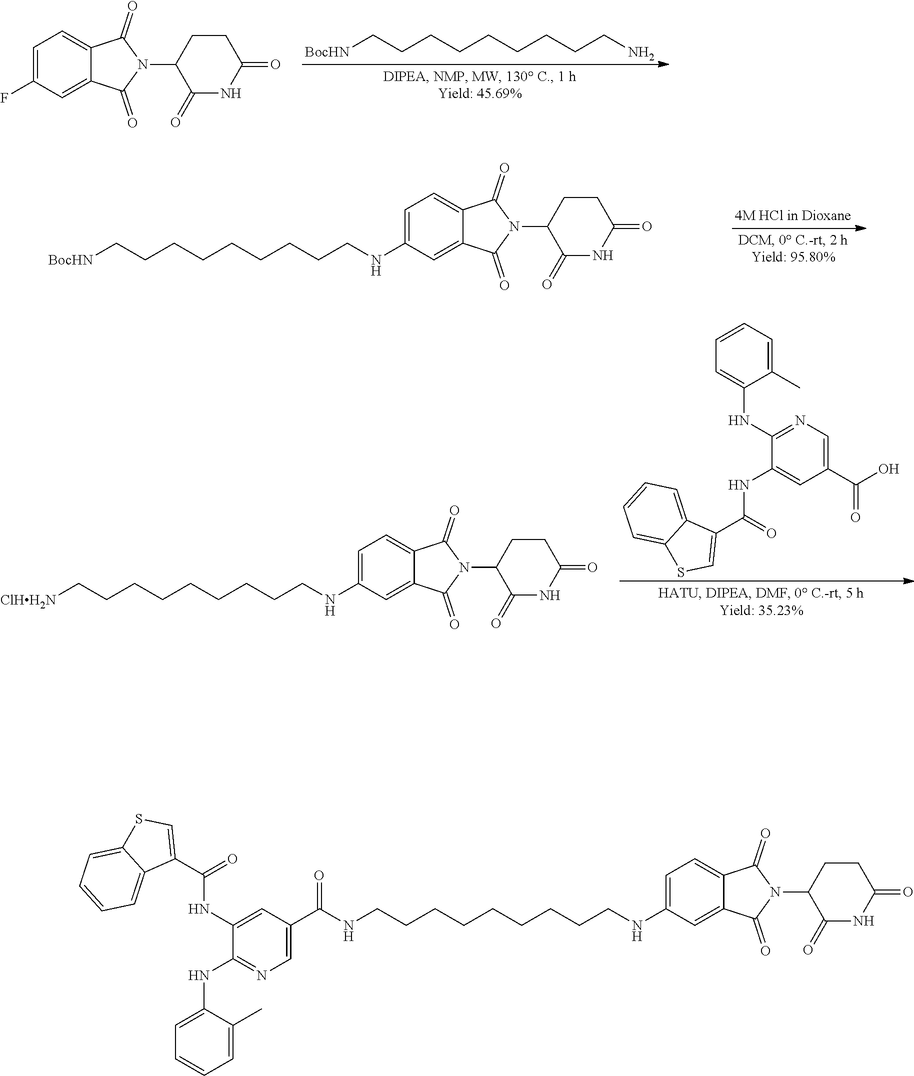

### Step 1. tert-butyl (9-((2-(2,6-dioxopiperidin-3-yl)-1,3-dioxoisoindolin-5-yl)amino)nonyl)carbamate

tert-butyl (9-((2-(2,6-dioxopiperidin-3-yl)-1,3-dioxoisoindolin-5-yl)amino)nonyl)carbamate was prepared from compound 2-(2,6-dioxopiperidin-3-yl)-5-fluoroisoindoline-1,3-dione (500 mg, 1.81 mmol) using Fluoro displacement reactions general procedure B and tert-butyl (9-aminononyl) carbamate 2 (554 mg, 1.99 mmol). Purification by MPLC (CombiFlash, 12 g column, packed with neutral alumina, Gradient Elusion, 2-3% methanol in DCM) provided tert-butyl (9-((2-(2,6-dioxopiperidin-3-yl)-1,3-dioxoisoindolin-5-yl)amino)nonyl)carbamate (850 mg, 45.69% yield) as a Greenish yellow sticky solid. LC-MS m/z 515.30 [M+H]⁺, Rt=0.901 min, HPLC: 57.96%, Rt=6.99 min.

### Step 2. 5-((9-aminononyl)amino)-2-(2,6-dioxopiperidin-3-yl)isoindoline-1,3-dione hydrochloride

5-((9-aminononyl)amino)-2-(2,6-dioxopiperidin-3-yl)isoindoline-1,3-dione hydrochloride was prepared from compound tert-butyl (9-((2-(2,6-dioxopiperidin-3-yl)-1,3-dioxoisoindolin-5-yl)amino)nonyl)carbamate (100 mg, 0.194 mmol) using Boc deprotection general procedure A (60 mg, 95.80% yield) as a yellow sticky solid. LC-MS m/z 415.20 [M+H]⁺, Rt=0.647 min, HPLC: 82.56%, Rt=6.68 min.

### Step 3. 5-(benzo[b]thiophene-3-carboxamido)-N-(9-((2-(2,6-dioxopiperidin-3-yl)-1,3-dioxoisoindolin-5-yl)amino)nonyl)-6-(o-tolylamino)nicotinamide

Compound 5-(benzo[b]thiophene-3-carboxamido)-N-(9-((2-(2,6-dioxopiperidin-3-yl)-1,3-dioxoisoindolin-5-yl)amino)nonyl)-6-(o-tolylamino)nicotinamide was prepared from compound 5-((9-aminononyl)amino)-2-(2,6-dioxopiperidin-3-yl)isoindoline-1,3-dione hydrochloride (50 mg, 0.111 mmol) using acid amine coupling general procedure A and 5-(benzo[b]thiophene-3-carboxamido)-6-(o-tolylamino)nicotinic acid (45 mg, 0.111 mmol). Purification with reversed-phase HPLC (Method A) provided 5-(benzo[b]thiophene-3-carboxamido)-N-(9-((2-(2,6-dioxopiperidin-3-yl)-1,3-dioxoisoindolin-5-yl)amino)nonyl)-6-(o-tolylamino)nicotinamide (31 mg, 35.23% yield) as a yellow solid. LC-MS m/z 800.50 [M+H]⁺, Rt=0.92 min, HPLC: 96.33%, Rt=6.91 min. ¹H NMR (400 MHz, DMSO-D₆) δ=11.08-11.03 (m, 1H), 10.05 (s, 1H), 8.70 (s, 1H), 8.51-8.47 (m, 1H), 8.45 (d, J=2.2 Hz, 1H), 8.31 (t, J=5.6 Hz, 1H), 8.18 (s, 1H), 8.13-8.07 (m, 2H), 7.55 (d, J=8.4 Hz, 1H), 7.52-7.42 (m, 3H), 7.24-7.15 (m, 2H), 7.07 (s, 2H), 6.93 (d, J=1.8 Hz, 2H), 6.83 (dd, J=2.0, 8.4 Hz, 1H), 5.02 (dd, J=5.4, 12.8 Hz, 1H), 3.24 (q, J=6.6 Hz, 2H), 3.14 (q, J=6.6 Hz, 2H), 2.93-2.81 (m, 1H), 2.59 (d, J=2.2 Hz, 2H), 2.17 (s, 3H), 2.04-1.94 (m, 1H), 1.60-1.46 (m, 4H), 1.36-1.26 (m, 9H) ppm.

### Example 4

### 5-(benzo[b]thiophene-3-carboxamido)-N-(10-(((2-(2,6-dioxopiperidin-3-yl)-1-oxoisoindolin-5-yl)methyl)amino)-10-oxodecyl)-6-(o-tolylamino)nicotinamide

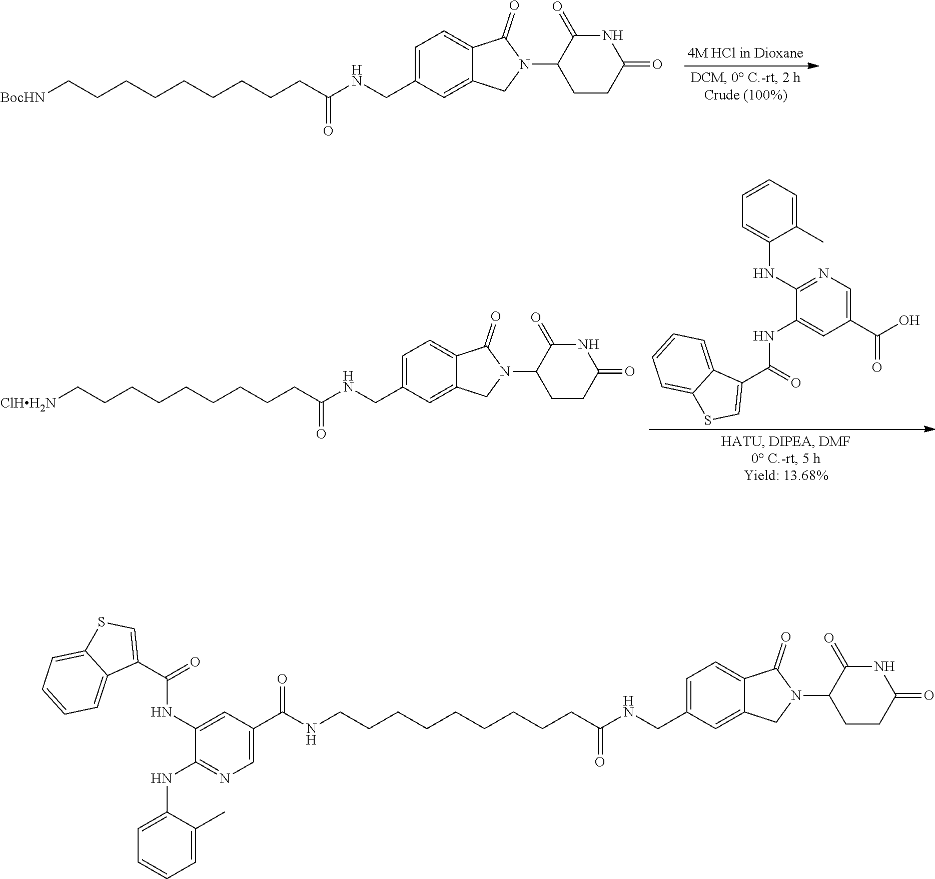

### Step 1. 10-amino-N-((2-(2,6-dioxopiperidin-3-yl)-1-oxoisoindolin-5-yl)methyl)decanamide hydrochloride

10-amino-N-((2-(2,6-dioxopiperidin-3-yl)-1-oxoisoindolin-5-yl)methyl)decanamide hydrochloride was prepared from tert-butyl (10-(((2-(2,6-dioxopiperidin-3-yl)-1-oxoisoindolin-5-yl)methyl)amino)-10-oxodecyl)carbamate (60 mg, 0.111 mmol) using Boc deprotection general procedure A (55 mg, 100% yield) as an off-white sticky solid. LC-MS m/z 443.35 [M+H]⁺, Rt=0.24 min, HPLC: 92.19%, Rt=5.75 min.

### Step 2. 5-(benzo[b]thiophene-3-carboxamido)-N-(10-(((2-(2,6-dioxopiperidin-3-yl)-1-oxoisoindolin-5-yl)methyl)amino)-10-oxodecyl)-6-(o-tolylamino)nicotinamide

5-(benzo[b]thiophene-3-carboxamido)-N-(10-(((2-(2,6-dioxopiperidin-3-yl)-1-oxoisoindolin-5-yl)methyl)amino)-10-oxodecyl)-6-(o-tolylamino)nicotinamide was prepared from compound 10-amino-N-((2-(2,6-dioxopiperidin-3-yl)-1-oxoisoindolin-5-yl)methyl)decanamide hydrochloride was prepared from tert-butyl (10-(((2-(2,6-dioxopiperidin-3-yl)-1-oxoisoindolin-5-yl)methyl)amino)-10-oxodecyl)carbamate (55 mg, 0.115 mmol) using acid amine coupling general procedure A and acid 5-(benzo[b]thiophene-3-carboxamido)-6-(o-tolylamino)nicotinic acid (46 mg, 0.115 mmol). Purification with reversed-phase HPLC (Method B) provided 5-(benzo[b]thiophene-3-carboxamido)-N-(10-(((2-(2,6-dioxopiperidin-3-yl)-1-oxoisoindolin-5-yl)methyl)amino)-10-oxodecyl)-6-(o-tolylamino)nicotinamide (13 mg, 13.68% yield) as a white solid. LC-MS m/z 828.55 [M+H]⁺, Rt=0.85 min, HPLC: 96.07%, Rt=7.51 min. ¹H NMR (400 MHz, DMSO-D₆) δ=11.13-10.83 (m, 1H), 10.57-10.26 (m, 1H), 8.75 (d, J=1.2 Hz, 1H), 8.53 (d, J=7.6 Hz, 2H), 8.44-8.40 (m, 2H), 8.32 (d, J=5.4 Hz, 1H), 8.18 (s, 1H), 8.08 (d, J=7.3 Hz, 1H), 7.66 (d, J=7.8 Hz, 1H), 7.54-7.41 (m, 4H), 7.37 (d, J=7.9 Hz, 1H), 7.24-7.13 (m, 2H), 7.07-6.99 (m, 1H), 5.10 (dd, J=5.1, 13.4 Hz, 1H), 4.48-4.24 (m, 4H), 3.22 (s, 2H), 2.97-2.84 (m, 1H), 2.62-2.58 (m, 2H), 2.42-2.35 (m, 1H), 2.22-2.10 (m, 5H), 2.04-1.94 (m, 1H), 1.51 (d, J=5.9 Hz, 4H), 1.26 (s, 9H) ppm.

### Example 5

### 5-(benzo[b]thiophene-3-carboxamido)-N-((S)-14-((2S,4R)-4-hydroxy-2-(((S)-1-(4-(4-methylthiazol-5-yl)phenyl)ethyl)carbamoyl)pyrrolidine-1-carbonyl)-15,15-dimethyl-12-oxo-3,6,9-trioxa-13-azahexadecyl)-6-(o-tolylamino)nicotinamide

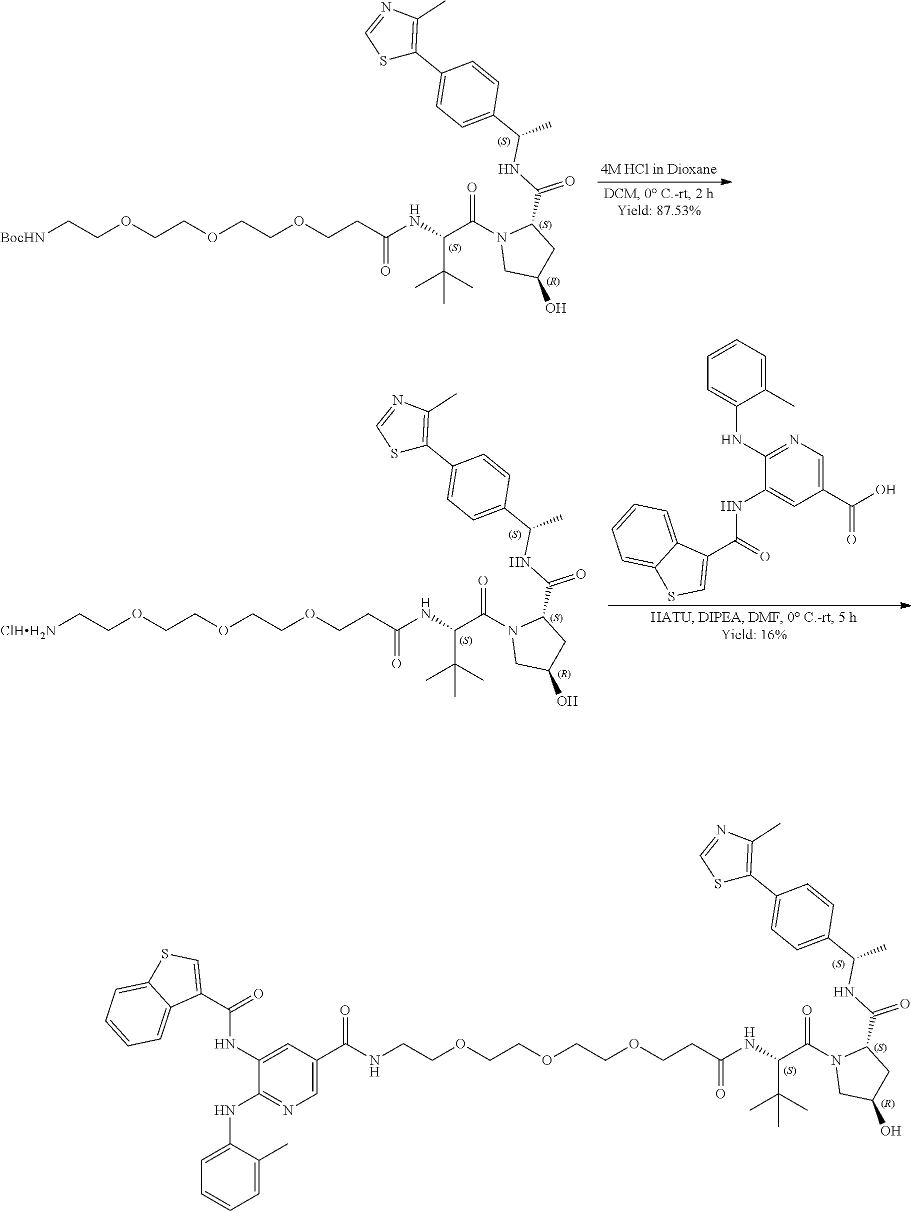

### Step 1. (2S,4R)-1-((S)-1-amino-14-(tert-butyl)-12-oxo-3,6,9-trioxa-13-azapentadecan-15-oyl)-4-hydroxy-N-((S)-1-(4-(4-methylthiazol-5-yl)phenyl)ethyl)pyrrolidine-2-carboxamide hydrochloride

(2S,4R)-1-((S)-1-amino-14-(tert-butyl)-12-oxo-3,6,9-trioxa-13-azapentadecan-15-oyl)-4-hydroxy-N-((S)-1-(4-(4-methylthiazol-5-yl)phenyl)ethyl)pyrrolidine-2-carboxamide hydrochloride was prepared from tert-butyl ((S)-14-((2S,4R)-4-hydroxy-2-(((S)-1-(4-(4-methylthiazol-5-yl)phenyl)ethyl)carbamoyl)pyrrolidine-1-carbonyl)-15,15-dimethyl-12-oxo-3,6,9-trioxa-13-azahexadecyl)carbamate (50 mg, 0.067 mmol) using Boc deprotection general procedure A (40 mg, 87.53% yield) as an Off-white sticky solid. LC-MS m/z 648.35 [M+H]⁺, Rt=0.78 min.

### Step 2. 5-(benzo[b]thiophene-3-carboxamido)-N-((S)-14-((2S,4R)-4-hydroxy-2-(((S)-1-(4-(4-methylthiazol-5-yl)phenyl)ethyl)carbamoyl)pyrrolidine-1-carbonyl)-15,15-dimethyl-12-oxo-3,6,9-trioxa-13-azahexadecyl)-6-(o-tolylamino)nicotinamide

5-(benzo[b]thiophene-3-carboxamido)-N-((S)-14-((2S,4R)-4-hydroxy-2-(((S)-1-(4-(4-methylthiazol-5-yl)phenyl)ethyl)carbamoyl)pyrrolidine-1-carbonyl)-15,15-dimethyl-12-oxo-3,6,9-trioxa-13-azahexadecyl)-6-(o-tolylamino)nicotinamide was prepared from (2S,4R)-1-((S)-1-amino-14-(tert-butyl)-12-oxo-3,6,9-trioxa-13-azapentadecan-15-oyl)-4-hydroxy-N-((S)-1-(4-(4-methylthiazol-5-yl)phenyl)ethyl)pyrrolidine-2-carboxamide hydrochloride (40 mg, 0.063 mmol) using acid amine coupling general procedure A and 5-(benzo[b]thiophene-3-carboxamido)-6-(o-tolylamino)nicotinic acid (20 mg, 0.050 mmol). Purification with reversed-phase HPLC (Method J) provided 5-(benzo[b]thiophene-3-carboxamido)-N-((S)-14-((2S,4R)-4-hydroxy-2-(((S)-1-(4-(4-methylthiazol-5-yl)phenyl)ethyl)carbamoyl)pyrrolidine-1-carbonyl)-15,15-dimethyl-12-oxo-3,6,9-trioxa-13-azahexadecyl)-6-(o-tolylamino)nicotinamide (11 mg, 16% yield) as a white solid. LC-MS m/z 1034.30 [M+H]⁺, Rt=1.50 min, HPLC: 91.35%, Rt=6.13 min. ¹H NMR (400 MHz, DMSO-D₆) δ=10.04 (s, 1H), 8.98 (s, 1H), 8.70 (s, 1H), 8.51-8.35 (m, 4H), 8.21 (s, 1H), 8.13 (d, J=2.1 Hz, 1H), 8.09 (dd, J=1.2, 7.1 Hz, 1H), 7.85 (d, J=9.4 Hz, 1H), 7.53-7.41 (m, 5H), 7.39-7.33 (m, 2H), 7.25-7.15 (m, 2H), 7.08-7.04 (m, 1H), 5.09 (d, J=2.9 Hz, 1H), 4.91 (t, J=7.2 Hz, 1H), 4.52 (d, J=9.4 Hz, 1H), 4.42 (t, J=8.0 Hz, 1H), 4.30-4.24 (m, 1H), 3.63-3.36 (m, 17H), 2.45 (s, 3H), 2.17 (s, 3H), 2.12-1.91 (m, 1H), 1.85-1.75 (m, 1H), 1.37 (d, J=7.0 Hz, 3H), 0.92 (s, 9H) ppm.

### Example 6

### 5-(benzo[b]thiophene-3-carboxamido)-N-(5-(2-(2,6-dioxopiperidin-3-yl)-1-oxoisoindolin-5-yl)pent-4-yn-1-yl)-6-(o-tolylamino)nicotinamide

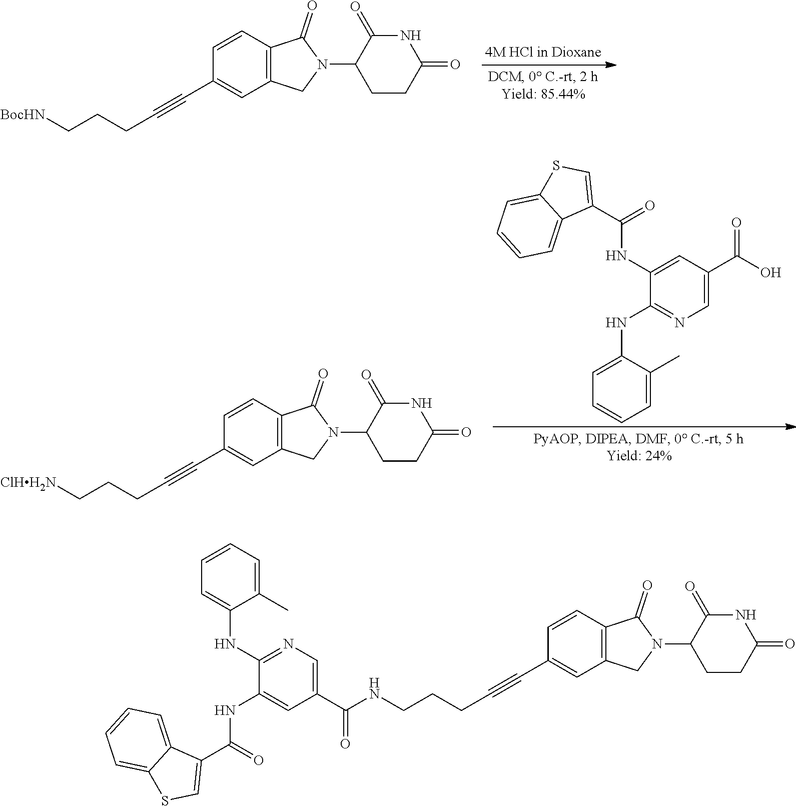

### Step 1. 3-(5-(5-aminopent-1-yn-1-yl)-1-oxoisoindolin-2-yl)piperidine-2,6-dione hydrochloride

3-(5-(5-aminopent-1-yn-1-yl)-1-oxoisoindolin-2-yl)piperidine-2,6-dione hydrochloride was prepared from tert-butyl (5-(2-(2,6-dioxopiperidin-3-yl)-1-oxoisoindolin-5-yl)pent-4-yn-1-yl)carbamate (50 mg, 0.12 mmol) using Boc deprotection general procedure A (40 mg, 85.44% yield) as an off-white sticky solid. LC-MS m/z 326.15 [M+H]⁺, Rt=0.14 min.

### Step 2. 5-(benzo[b]thiophene-3-carboxamido)-N-(5-(2-(2,6-dioxopiperidin-3-yl)-1-oxoisoindolin-5-yl)pent-4-yn-1-yl)-6-(o-tolylamino)nicotinamide

5-(benzo[b]thiophene-3-carboxamido)-N-(5-(2-(2,6-dioxopiperidin-3-yl)-1-oxoisoindolin-5-yl)pent-4-yn-1-yl)-6-(o-tolylamino)nicotinamide was prepared from 3-(5-(5-aminopent-1-yn-1-yl)-1-oxoisoindolin-2-yl)piperidine-2,6-dione hydrochloride (39 mg, 0.11 mmol) using acid amine coupling general procedure B and 5-(benzo[b]thiophene-3-carboxamido)-6-(o-tolylamino)nicotinic acid (40 mg, 0.099 mmol). Purification with reversed-phase HPLC (Method F) provided 5-(benzo[b]thiophene-3-carboxamido)-N-(5-(2-(2,6-dioxopiperidin-3-yl)-1-oxoisoindolin-5-yl)pent-4-yn-1-yl)-6-(o-tolylamino)nicotinamide (18 mg, 24% yield) as a white solid. LC-MS m/z 711.65 [M+H]⁺, Rt=1.48 min, HPLC: 95.54%, Rt=5.98 min. ¹H NMR (400 MHz, DMSO-D₆) δ=10.07 (s, 1H), 8.98 (s, 1H), 8.72-8.67 (m, 1H), 8.52-8.47 (m, 1H), 8.45-8.43 (m, 1H), 8.39-8.30 (m, 2H), 8.25 (s, 1H), 8.14-8.07 (m, 2H), 7.79 (d, J=9.4 Hz, 1H), 7.52-7.42 (m, 5H), 7.39-7.35 (m, 2H), 7.25-7.16 (m, 2H), 7.10-7.04 (m, 1H), 4.91 (quin, J=7.2 Hz, 1H), 4.51 (d, J=9.2 Hz, 1H), 4.45-4.39 (m, 1H), 4.27 (d, J=2.6 Hz, 1H), 3.64-3.57 (m, 2H), 3.28-3.20 (m, 3H), 2.45 (s, 3H), 2.29-2.21 (m, 1H), 2.18 (s, 3H), 2.16-2.09 (m, 1H), 2.01 (ddd, J=2.4, 7.7, 12.6 Hz, 1H), 1.82-1.74 (m, 1H), 1.57-1.45 (m, 4H), 1.37 (d, J=7.0 Hz, 3H), 1.32-1.22 (m, 2H), 0.92 (s, 9H) ppm.

### Example 7

### 5-(benzo[b]thiophene-3-carboxamido)-N-(4-(4-(2-(2,6-dioxopiperidin-3-yl)-1,3-dioxoisoindolin-5-yl)piperazin-1-yl)phenyl)-6-(o-tolylamino)nicotinamide

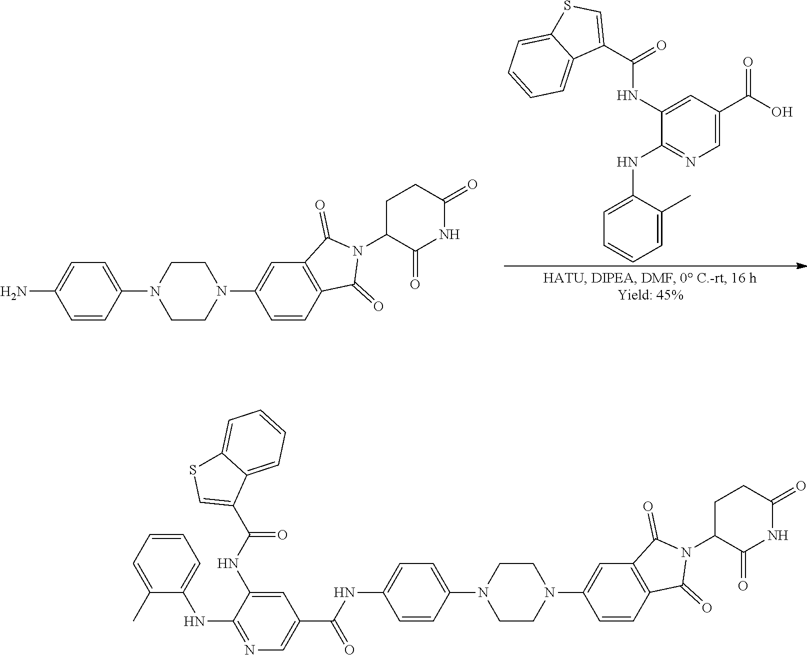 [SMILES: Nc1ccc(N2CCN(c3ccc4c(c3)C(=O)N(C3CCC(=O)NC3=O)C4=O)CC2)cc1 ; Cc1ccccc1Nc1ncc(C(=O)O)cc1NC(=O)c1csc2ccccc12 ; Cc1ccccc1Nc1ncc(C(=O)Nc2ccc(N3CCN(c4ccc5c(c4)C(=O)N(C4CCC(=O)NC4=O)C5=O)CC3)cc2)cc1NC(=O)c1csc2ccccc12]

5-(benzo[b]thiophene-3-carboxamido)-N-(4-(4-(2-(2,6-dioxopiperidin-3-yl)-1,3-dioxoisoindolin-5-yl)piperazin-1-yl)phenyl)-6-(o-tolylamino)nicotinamide was prepared from 5-(4-(4-aminophenyl)piperazin-1-yl)-2-(2,6-dioxopiperidin-3-yl)isoindoline-1,3-dione (50 mg, 0.12 mmol) using acid amine coupling general procedure A and 5-(benzo[b]thiophene-3-carboxamido)-6-(o-tolylamino)nicotinic acid (47 mg, 0.12 mmol). Purification with reversed-phase HPLC (Method F) provided 5-(benzo[b]thiophene-3-carboxamido)-N-(4-(4-(2-(2,6-dioxopiperidin-3-yl)-1,3-dioxoisoindolin-5-yl)piperazin-1-yl)phenyl)-6-(o-tolylamino)nicotinamide (44 mg, 45% yield) as a yellow solid. LC-MS m/z 819.50 [M+H]⁺, Rt=0.93 min, HPLC: 96.48%, Rt=6.52 min. ¹H NMR (400 MHz, DMSO-D₆) δ=11.08 (s, 1H), 10.14 (s, 1H), 10.01 (s, 1H), 8.73 (s, 1H), 8.58-8.48 (m, 3H), 8.28 (d, J=1.9 Hz, 1H), 8.10 (td, J=1.0, 7.9 Hz, 1H), 7.72 (d, J=8.5 Hz, 1H), 7.65 (d, J=9.1 Hz, 2H), 7.54-7.39 (m, 4H), 7.34 (dd, J=2.1, 8.7 Hz, 1H), 7.28-7.18 (m, 2H), 7.13 (d, J=6.7 Hz, 1H), 7.08-7.02 (m, 2H), 5.12-5.05 (m, 1H), 3.64 (s, 4H), 3.30 (s, 4H), 2.95-2.81 (m, 1H), 2.63-2.55 (m, 2H), 2.21 (s, 3H), 2.06-1.96 (m, 1H) ppm.

### Example 8

### 5-(benzo[b]thiophene-3-carboxamido)-6-((2-chloro-5-fluorophenyl)amino)-N-(5-((2-(2,6-dioxopiperidin-3-yl)-1,3-dioxoisoindolin-5-yl)amino)pentyl)nicotinamide

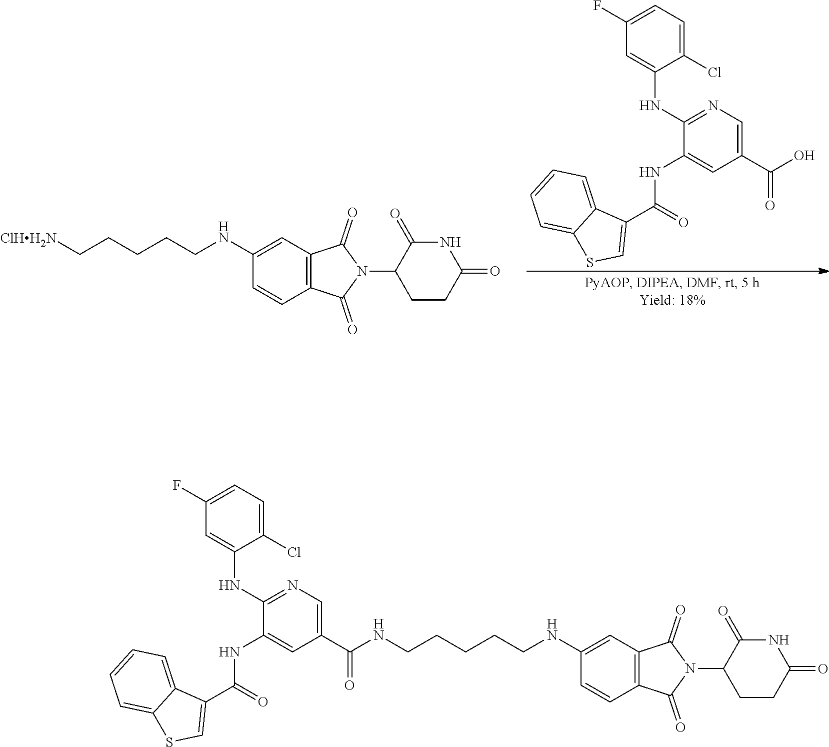

5-(benzo[b]thiophene-3-carboxamido)-6-((2-chloro-5-fluorophenyl)amino)-N-(5-((2-(2,6-dioxopiperidin-3-yl)-1,3-dioxoisoindolin-5-yl)amino)pentyl)nicotinamide was prepared from 5-((5-aminopentyl)amino)-2-(2,6-dioxopiperidin-3-yl)isoindoline-1,3-dione hydrochloride (35 mg, 0.098 mmol) using acid amine coupling general procedure B and 5-(benzo[b]thiophene-3-carboxamido)-6-((2-chloro-5-fluorophenyl)amino)nicotinic acid (34 mg, 0.077 mmol). Purification with reversed-phase HPLC (Method N) provided 5-(benzo[b]thiophene-3-carboxamido)-6-((2-chloro-5-fluorophenyl)amino)-N-(5-((2-(2,6-dioxopiperidin-3-yl)-1,3-dioxoisoindolin-5-yl)amino)pentyl)nicotinamide (13.5 mg, 18% yield) as a white solid. LC-MS m/z 782.15 [M+H]⁺, Rt=0.95 min, HPLC: 98.14%, Rt=7.49 min. ¹H NMR (400 MHz, DMSO-D₆) δ=11.05 (s, 1H), 10.51 (s, 1H), 8.74 (s, 1H), 8.70 (d, J=2.1 Hz, 1H), 8.56-8.46 (m, 2H), 8.33 (s, 1H), 8.25-8.09 (m, 3H), 7.57-7.46 (m, 4H), 7.12 (t, J=5.2 Hz, 1H), 6.98-6.82 (m, 3H), 5.02 (dd, J=5.4, 12.8 Hz, 1H), 3.31-3.28 (m, 2H), 3.21-3.13 (m, 2H), 2.92-2.80 (m, 1H), 2.58 (d, J=2.5 Hz, 2H), 2.03-1.93 (m, 1H), 1.67-1.54 (m, 4H), 1.49-1.38 (m, 2H) ppm.

### Example 9

### 5-(benzo[b]thiophene-3-carboxamido)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-6-(o-tolylamino)nicotinamide

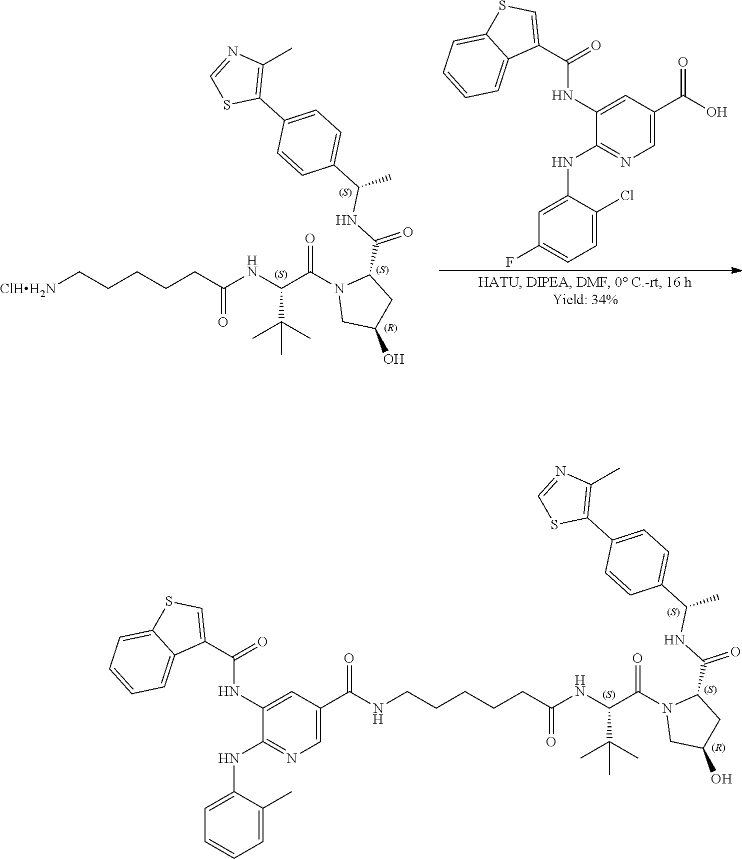

5-(benzo[b]thiophene-3-carboxamido)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-6-(o-tolylamino)nicotinamide was prepared from (2S,4R)-1-((S)-2-(6-aminohexanamido)-3,3-dimethylbutanoyl)-4-hydroxy-N-((S)-1-(4-(4-methylthiazol-5-yl)phenyl)ethyl)pyrrolidine-2-carboxamide hydrochloride (37 mg, 0.062 mmol) using acid amine coupling general procedure A and 5-(benzo[b]thiophene-3-carboxamido)-6-((2-chloro-5-fluorophenyl)amino)nicotinic acid (25 mg, 0.062 mmol). Purification with reversed-phase HPLC (Method Q) provided 5-(benzo[b]thiophene-3-carboxamido)-N-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)-6-(o-tolylamino)nicotinamide (20 mg, 34% yield) as a white solid. LC-MS m/z 943.35 [M+H]⁺, Rt=0.90 min, HPLC: 98.42%, Rt=6.34 min. ¹H NMR (400 MHz, DMSO-D₆) δ=10.07 (s, 1H), 8.98 (s, 1H), 8.72-8.67 (m, 1H), 8.52-8.47 (m, 1H), 8.45-8.43 (m, 1H), 8.39-8.30 (m, 2H), 8.25 (s, 1H), 8.14-8.07 (m, 2H), 7.79 (d, J=9.4 Hz, 1H), 7.52-7.42 (m, 5H), 7.39-7.35 (m, 2H), 7.25-7.16 (m, 2H), 7.10-7.04 (m, 1H), 4.91 (quin, J=7.2 Hz, 1H), 4.51 (d, J=9.2 Hz, 1H), 4.45-4.39 (m, 1H), 4.27 (d, J=2.6 Hz, 1H), 3.64-3.57 (m, 2H), 3.28-3.20 (m, 3H), 2.45 (s, 3H), 2.29-2.21 (m, 1H), 2.18 (s, 3H), 2.16-2.09 (m, 1H), 2.01 (ddd, J=2.4, 7.7, 12.6 Hz, 1H), 1.82-1.74 (m, 1H), 1.57-1.45 (m, 4H), 1.37 (d, J=7.0 Hz, 3H), 1.32-1.22 (m, 2H), 0.92 (s, 9H) ppm.

### Example 10

### (1S)-1-(2-chloro-5-fluorophenyl)-N-(7-(2-(2,6-dioxopiperidin-3-yl)-1-oxoisoindoline-5-carboxamido)heptyl)-7-(3-fluoro-5-(trifluoromethyl)benzamido)-3-oxoisoindoline-5-carboxamide

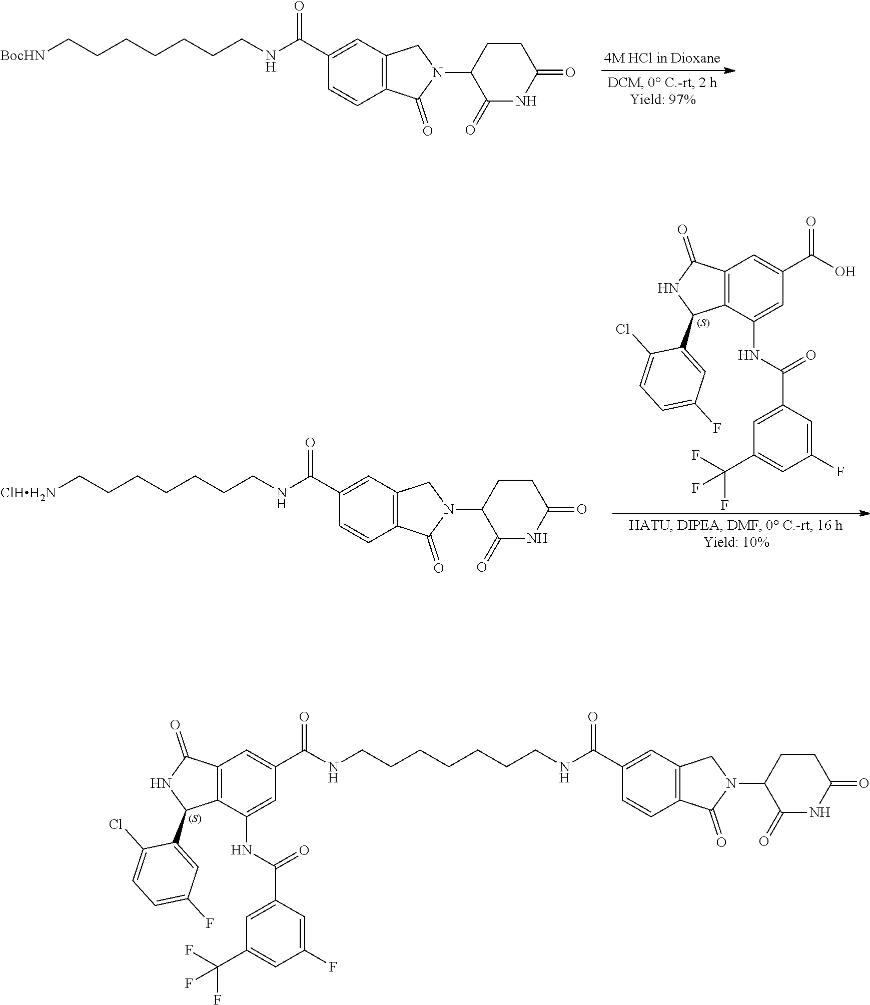

### Step 1. N-(7-aminoheptyl)-2-(2,6-dioxopiperidin-3-yl)-1-oxoisoindoline-5-carboxamide hydrochloride

N-(7-aminoheptyl)-2-(2,6-dioxopiperidin-3-yl)-1-oxoisoindoline-5-carboxamide hydrochloride was prepared from tert-butyl (7-(2-(2,6-dioxopiperidin-3-yl)-1-oxoisoindoline-5-carboxamido)heptyl)carbamate (80 mg, 0.16 mmol) using Boc deprotection general procedure A (70 mg, 97% yield) as an off-white sticky solid. LC-MS m/z 401.20 [M+H]⁺, Rt=0.16 min.

### Step 2. (1S)-1-(2-chloro-5-fluorophenyl)-N-(7-(2-(2,6-dioxopiperidin-3-yl)-1-oxoisoindoline-5-carboxamido)heptyl)-7-(3-fluoro-5-(trifluoromethyl)benzamido)-3-oxoisoindoline-5-carboxamide

(1S)-1-(2-chloro-5-fluorophenyl)-N-(7-(2-(2,6-dioxopiperidin-3-yl)-1-oxoisoindoline-5-carboxamido)heptyl)-7-(3-fluoro-5-(trifluoromethyl)benzamido)-3-oxoisoindoline-5-carboxamide was prepared from N-(7-aminoheptyl)-2-(2,6-dioxopiperidin-3-yl)-1-oxoisoindoline-5-carboxamide hydrochloride was prepared from tert-butyl (34 mg, 0.078 mmol) using acid amine coupling general procedure A and (S)-1-(2-chloro-5-fluorophenyl)-7-(3-fluoro-5-(trifluoromethyl)benzamido)-3-oxoisoindoline-5-carboxylic acid (40 mg, 0.078 mmol). Purification with reversed-phase HPLC (Method C) provided (1S)-1-(2-chloro-5-fluorophenyl)-N-(7-(2-(2,6-dioxopiperidin-3-yl)-1-oxoisoindoline-5-carboxamido)heptyl)-7-(3-fluoro-5-(trifluoromethyl)benzamido)-3-oxoisoindoline-5-carboxamide (9 mg, 10% yield) as a white solid. LC-MS m/z 893.15 [M+H]⁺, Rt=0.83 min, HPLC: 90.13%, Rt=5.75 min, Chiral HPLC: 50.27%, Rt=6.71 min and 48.86, Rt=7.91 min. ¹H NMR (400 MHz, DMSO-D₆) δ=11.00 (s, 1H), 10.61 (s, 1H), 9.29-9.20 (m, 1H), 8.79 (t, J=5.4 Hz, 1H), 8.67-8.62 (m, 1H), 8.22 (s, 1H), 8.04 (s, 1H), 8.00-7.93 (m, 3H), 7.81-7.71 (m, 2H), 7.69-7.65 (m, 1H), 7.34-7.28 (m, 1H), 7.13-7.05 (m, 1H), 6.09-5.95 (m, 1H), 5.14 (dd, J=5.2, 13.3 Hz, 1H), 4.55-4.46 (m, 1H), 4.43-4.33 (m, 1H), 3.30-3.23 (m, 4H), 2.99-2.87 (m, 1H), 2.65-2.54 (m, 2H), 2.45-2.37 (m, 1H), 2.07-1.95 (m, 1H), 1.55 (br s, 4H), 1.35 (br s, 6H) ppm.

### Example 11

### 1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide

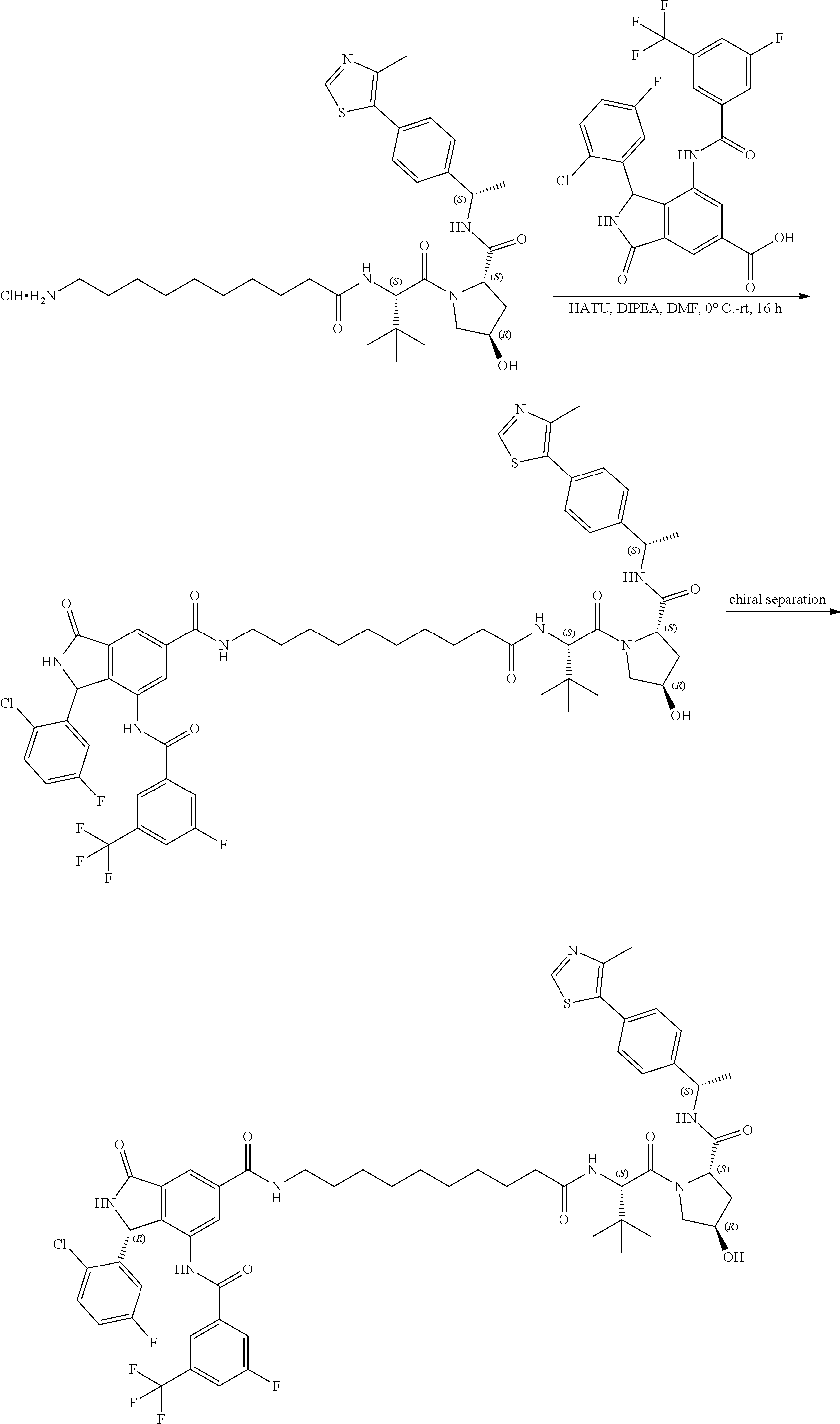

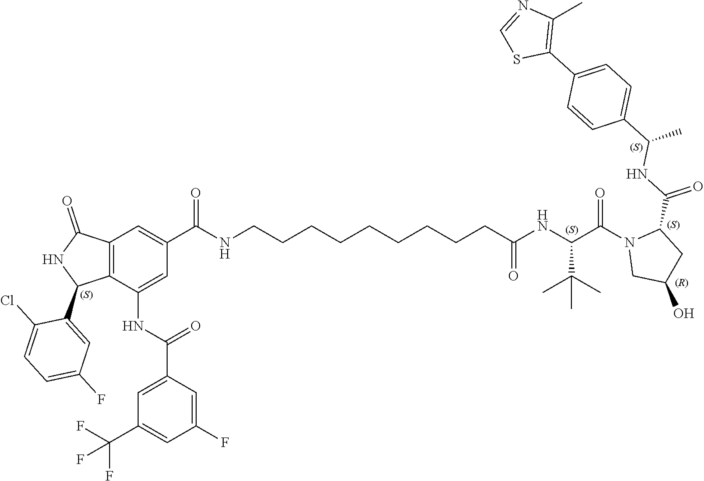

### Step 1. 1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide

1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide was prepared from (2S,4R)-1-((S)-2-(10-aminodecanamido)-3,3-dimethylbutanoyl)-4-hydroxy-N-((S)-1-(4-(4-methylthiazol-5-yl)phenyl)ethyl)pyrrolidine-2-carboxamide hydrochloride (81 mg, 0.13 mmol) using acid amine coupling general procedure A and 1-(2-chloro-5-fluorophenyl)-7-(3-fluoro-5-(trifluoromethyl)benzamido)-3-oxoisoindoline-5-carboxylic acid (80 mg, 0.16 mmol). Purification with reversed-phase HPLC (Method Q) provided 1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide.

### (R)-1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide

The racemic compound was separated by chiral purification (Method B) providing (R)-1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide (Peak 1, 11 mg, as a white solid) LC-MS m/z 1106.45 [M+H]⁺, Rt=0.93 min, HPLC: 98.82%, Rt=6.67 min, Chiral HPLC: 99.47%, Rt=4.99 min. ¹H NMR (400 MHz, DMSO-D₆) δ=10.61 (s, 1H), 9.32-9.18 (m, 1H), 8.98 (s, 1H), 8.78 (t, J=5.4 Hz, 1H), 8.36 (d, J=7.8 Hz, 1H), 8.27-8.20 (m, 1H), 8.04-7.92 (m, 2H), 7.79-7.67 (m, 3H), 7.47-7.24 (m, 6H), 7.17-7.03 (m, 1H), 6.13-5.94 (m, 1H), 5.09 (d, J=3.5 Hz, 1H), 4.99-4.80 (m, 1H), 4.53-4.47 (m, 1H), 4.42 (t, J=8.0 Hz, 1H), 4.27 (d, J=3.2 Hz, 1H), 3.63-3.55 (m, 2H), 2.45 (s, 3H), 2.29-2.19 (m, 1H), 2.15-2.06 (m, 1H), 2.03-1.96 (m, 2H), 1.82-1.75 (m, 1H), 1.56-1.45 (m, 4H), 1.37 (d, J=6.9 Hz, 3H), 1.27-1.22 (m, 11H), 0.93 (s, 9H) ppm.

### (S)-1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide

The racemic compound was separated by chiral purification (Method B) providing (S)-1-(2-chloro-5-fluorophenyl)-7-(3-fluoro-5-(trifluoromethyl)benzamido)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)-3-oxoisoindoline-5-carboxamide (Peak 2, 10 mg, as a white solid). LC-MS m/z 1106.35 [M+H]⁺, Rt=0.93 min, HPLC: 97.45%, Rt=6.67 min, Chiral HPLC: 99.35%, Rt=6.34 min. ¹H NMR (400 MHz, DMSO-D₆) δ=10.62 (br s, 1H), 9.25 (br s, 1H), 8.98 (s, 1H), 8.78 (t, J=5.6 Hz, 1H), 8.37 (d, J=7.9 Hz, 1H), 8.22 (s, 1H), 8.02-7.93 (m, 2H), 7.80-7.72 (m, 2H), 7.68 (s, 1H), 7.45-7.29 (m, 6H), 7.12-7.05 (m, 1H), 6.09-5.97 (m, 1H), 5.09 (d, J=3.5 Hz, 1H), 4.96 (s, 1H), 4.54-4.47 (m, 1H), 4.43-4.37 (m, 1H), 4.29-4.23 (m, 1H), 3.63-3.57 (m, 2H), 2.45 (s, 3H), 2.27-2.19 (m, 1H), 2.14-2.07 (m, 1H), 2.04-1.94 (m, 2H), 1.79 (ddd, J=4.7, 8.4, 12.7 Hz, 1H), 1.57-1.47 (m, 4H), 1.37 (d, J=6.9 Hz, 3H), 1.28-1.23 (m, 11H), 0.92 (s, 9H) ppm.

### Example 12

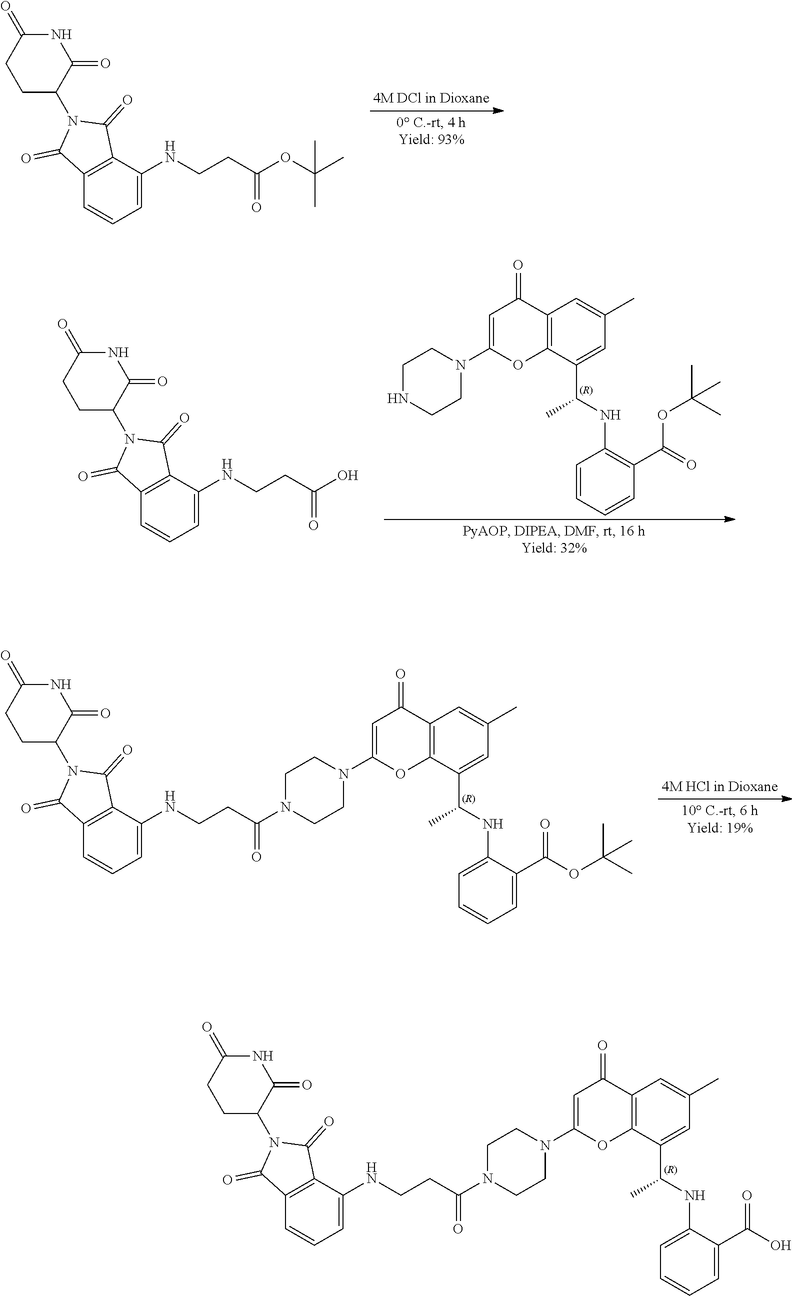

### Step 1. 3-((2-(2,6-dioxopiperidin-3-yl)-1,3-dioxoisoindolin-4-yl)amino)propanoic acid

3-((2-(2,6-dioxopiperidin-3-yl)-1,3-dioxoisoindolin-4-yl)amino)propanoic acid was prepared from tert-butyl 3-((2-(2,6-dioxopiperidin-3-yl)-1,3-dioxoisoindolin-4-yl)amino)propanoate (100 mg, 0.249 mmol) using tert-butyl ester hydrolysis general procedure A (86 mg, 93% yield) as a yellow sticky solid. LC-MS m/z 345.80 [M+H]⁺, Rt=1.35 min, HPLC: 84.53%, Rt=5.48 min

### Step 2. tert-butyl 2-(((1R)-1-(2-(4-(3-((2-(2,6-dioxopiperidin-3-yl)-1,3-dioxoisoindolin-4-yl)amino)propanoyl)piperazin-1-yl)-6-methyl-4-oxo-4H-chromen-8-yl)ethyl)amino)benzoate

tert-butyl 2-(((1R)-1-(2-(4-(3-((2-(2,6-dioxopiperidin-3-yl)-1,3-dioxoisoindolin-4-yl)amino)propanoyl)piperazin-1-yl)-6-methyl-4-oxo-4H-chromen-8-yl)ethyl)amino)benzoate was prepared from tert-butyl (R)-2-((1-(6-methyl-4-oxo-2-(piperazin-1-yl)-4H-chromen-8-yl)ethyl)amino)benzoate (43 mg, 0.093 mmol) using acid amine coupling general procedure B and 3-((2-(2,6-dioxopiperidin-3-yl)-1,3-dioxoisoindolin-4-yl)amino)propanoic acid (40 mg, 0.12 mmol). After completion of reaction, the reaction mass was quenched with ice cooled water (10 mL), stir for 2 hours, filter the precipitated solid and dry to obtain compound 4 (35 mg, 32% yield) as a yellow solid. LC-MS m/z 791.20 [M+H]⁺, Rt=1.59 min, HPLC: 84.72%, Rt=7.42 min.

### Step 3. 2-(((1R)-1-(2-(4-(3-((2-(2,6-dioxopiperidin-3-yl)-1,3-dioxoisoindolin-4-yl)amino)propanoyl)piperazin-1-yl)-6-methyl-4-oxo-4H-chromen-8-yl)ethyl)amino)benzoic acid

2-(((1R)-1-(2-(4-(3-((2-(2,6-dioxopiperidin-3-yl)-1,3-dioxoisoindolin-4-yl)amino)propanoyl)piperazin-1-yl)-6-methyl-4-oxo-4H-chromen-8-yl)ethyl)amino)benzoic acid was prepared from tert-butyl 2-(((1R)-1-(2-(4-(3-((2-(2,6-dioxopiperidin-3-yl)-1,3-dioxoisoindolin-4-yl)amino)propanoyl)piperazin-1-yl)-6-methyl-4-oxo-4H-chromen-8-yl)ethyl)amino)benzoate (50 mg, 0.063 mmol) using tert-butyl ester hydrolysis general procedure A. Purification with reversed-phase HPLC (Method L) provided 5 (9.2 mg, 19% yield) as a yellow solid. LC-MS m/z 735.15 [M+H]⁺, Rt=1.42 min, HPLC: 98.27%, Rt=6.16 min, Chiral HPLC: 45.75%, Rt=9.36 min and 54.25%, Rt=18.46 min. ¹H NMR (400 MHz, DMSO-D₆, 25° C.): δ=12.77 (br s, 1H), 11.09 (s, 1H), 8.35 (d, J=6.5 Hz, 1H), 7.81 (dd, J=7.8, 1.7 Hz, 1H), 7.56-7.65 (m, 2H), 7.38 (d, J=2.1 Hz, 1H), 7.11-7.31 (m, 2H), 7.03 (d, J=7.0 Hz, 1H), 6.79 (t, J=6.3 Hz, 1H), 6.55 (t, J=7.2 Hz, 1H), 6.46 (d, J=8.4 Hz, 1H), 5.53 (s, 1H), 4.94-5.18 (m, 2H), 3.51-3.71 (m, 10H), 2.82-2.93 (m, 1H), 2.72 (t, J=5.9 Hz, 2H), 2.59 (br s, 1H), 2.30 (s, 3H), 1.95-2.03 (m, 2H), 1.58 ppm (d, J=6.6 Hz, 3H) ppm.

### Example 13

### (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoic acid

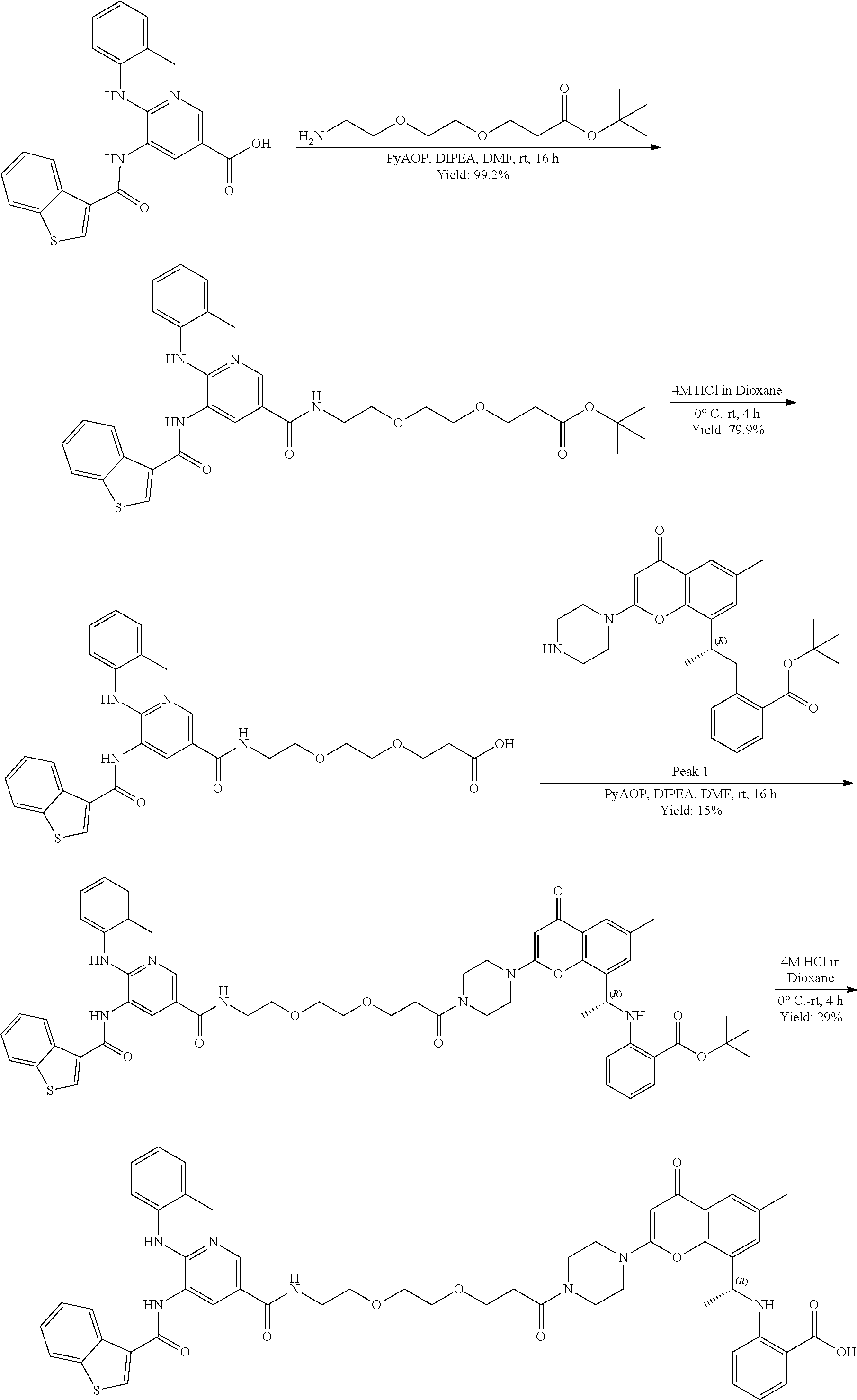

### Step 1. tert-butyl 3-(2-(2-(5-(benzo[b]thiophene-3-carboxamido)-6-(o-tolylamino)nicotinamido)ethoxy)ethoxy)propanoate

tert-butyl 3-(2-(2-(5-(benzo[b]thiophene-3-carboxamido)-6-(o-tolylamino)nicotinamido)ethoxy)ethoxy)propanoate was prepared from tert-butyl 3-(2-(2-aminoethoxy)ethoxy)propanoate (139 mg, 0.595 mmol) using acid amine coupling general procedure B and 5-(benzo[b]thiophene-3-carboxamido)-6-(o-tolylamino)nicotinic acid (200 mg, 0.496 mmol). Purification with column chromatography using a Redi Sep 4 g SiO₂ column. It was eluted with a gradient of 50-70% of ethyl acetate in hexane to obtain compound-15 (320 mg, 99.2% yield) as an off-white solid. LC-MS m/z 619.10 [M+H]⁺, Rt=1.59 min, HPLC: 95.11%, Rt=6.95 min.

### Step 2. 3-(2-(2-(5-(benzo[b]thiophene-3-carboxamido)-6-(o-tolylamino)nicotinamido)ethoxy)ethoxy)propanoic acid

3-(2-(2-(5-(benzo[b]thiophene-3-carboxamido)-6-(o-tolylamino)nicotinamido)ethoxy)ethoxy)propanoic acid was prepared from tert-butyl 3-(2-(2-(5-(benzo[b]thiophene-3-carboxamido)-6-(o-tolylamino)nicotinamido)ethoxy)ethoxy)propanoate (300 mg, 0.485 mmol) using tert-butyl ester hydrolysis general procedure A (250 mg, 79.9% yield) as an off-white sticky solid. LC-MS m/z 563.25 [M+H]⁺, Rt=1.46 min, HPLC: 87.22%, Rt=5.63 min.

### Step 3. tert-butyl (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoate

tert-butyl (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoate was prepared from tert-butyl (R)-2-((1-(6-methyl-4-oxo-2-(piperazin-1-yl)-4H-chromen-8-yl)ethyl)amino)benzoate (40 mg, 0.086 mmol) using acid amine coupling general procedure B and 3-(2-(2-(5-(benzo[b]thiophene-3-carboxamido)-6-(o-tolylamino)nicotinamido)ethoxy)ethoxy)propanoic acid was prepared from tert-butyl 3-(2-(2-(5-(benzo[b]thiophene-3-carboxamido)-6-(o-tolylamino)nicotinamido)ethoxy)ethoxy)propanoate (49 mg, 0.086 mmol). After completion of reaction, the reaction mass was quenched with ice cooled water (10 mL), stir for 2 hours, filter the precipitated solid and dry to obtain compound-17 (40 mg, 15% yield) as an off-white solid. LC-MS m/z 1008.25 [M+H]⁺, Rt=1.68 min.

### Step 4. (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoic acid

(R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoic acid was prepared from tert-butyl (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoate (40 mg, 0.040 mmol) using tert-butyl ester hydrolysis general procedure A. Purification with reversed-phase HPLC (Method R) provided (R)-2-((1-(2-(4-(3-(2-(2-(5-(benzo[b]thiophene-3-carboxamido)-6-(o-tolylamino)nicotinamido)ethoxy)ethoxy)propanoyl)piperazin-1-yl)-6-methyl-4-oxo-4H-chromen-8-yl)ethyl)amino)benzoic acid (11.2 mg, 29% yield) as a white solid. LC-MS m/z 952.30 [M+H]⁺, Rt=1.49 min, HPLC: 99.00%, Rt=6.83 min, Chiral HPLC: 99.41%, Rt=7.96 min. ¹H NMR (400 MHz, DMSO-D₆, 25° C.): δ=12.77 (br s, 1H), 10.06 (br s, 1H), 8.71 (br s, 1H), 8.46-8.51 (m, 1H), 8.45 (d, J=2.1 Hz, 1H), 8.40 (t, J=5.5 Hz, 1H), 8.19-8.25 (m, 1H), 8.13 (d, J=1.6 Hz, 1H), 8.08 (dd, J=6.9, 1.2 Hz, 1H), 7.80 (dd, J=7.9, 1.6 Hz, 1H), 7.60 (d, J=1.5 Hz, 1H), 7.41-7.51 (m, 3H), 7.38 (d, J=1.8 Hz, 1H), 7.13-7.25 (m, 3H), 7.01-7.07 (m, 1H), 6.53 (t, J=7.3 Hz, 1H), 6.45 (d, J=8.2 Hz, 1H), 5.51 (s, 1H), 5.05-5.13 (m, 1H), 3.64 (t, J=6.6 Hz, 2H), 3.59 (br s, 6H), 3.50-3.55 (m, 8H), 3.40-3.43 (m, 2H), 2.60 (br s, 2H), 2.30 (s, 3H), 2.16 (s, 3H), 1.95-2.04 (m, 1H), 1.57 ppm (d, J=6.6 Hz, 3H) ppm.

### Example 14

### N-((R)-1-(2-chloro-5-fluorophenyl)-6-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamoyl)-3-oxo-1,2,3,4-tetrahydropyrrolo[1,2-a]pyrazin-8-yl)benzo[d]isothiazole-3-carboxamide

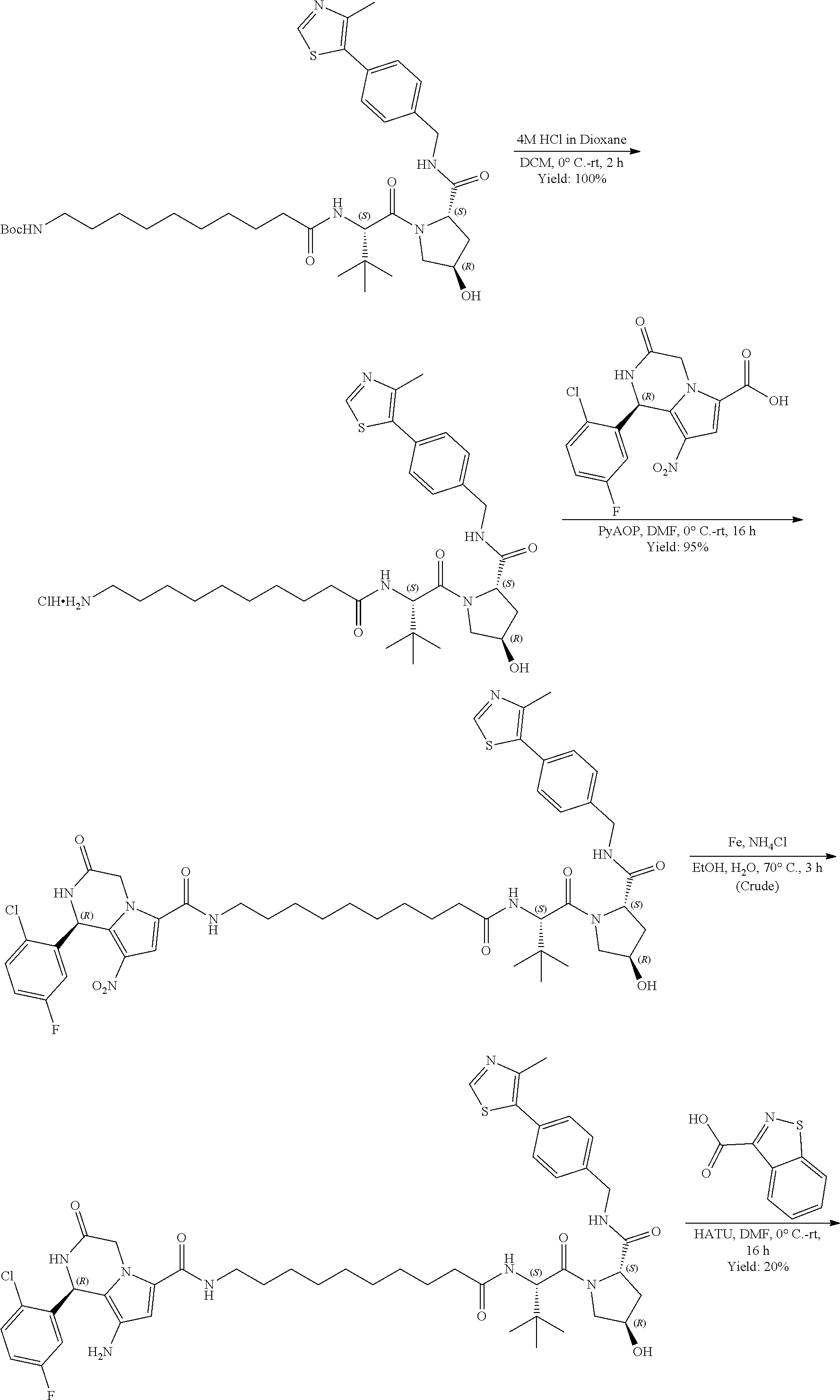

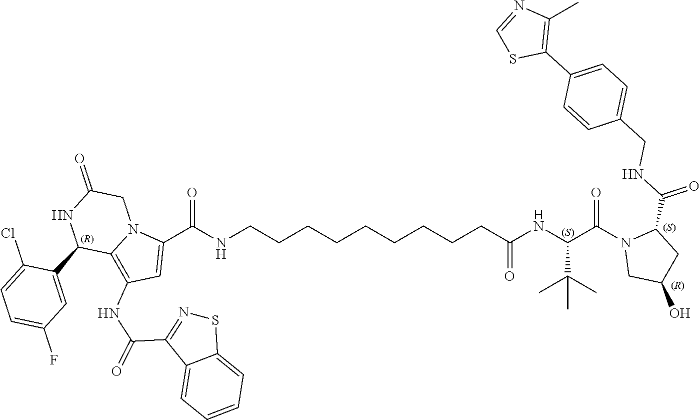

### Step 1. (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide hydrochloride

(2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide hydrochloride was prepared from tert-butyl (10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamate (30 mg, 0.043 mmol) using Boc deprotection general procedure A (30 mg, 100% yield) as an off-white sticky solid. LC-MS m/z 600.45 [M+H]⁺, Rt=0.71 min.

### Step 2. (R)-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-8-nitro-3-oxo-1,2,3,4-tetrahydropyrrolo[1,2-a]pyrazine-6-carboxamide

(R)-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-8-nitro-3-oxo-1,2,3,4-tetrahydropyrrolo[1,2-a]pyrazine-6-carboxamide was prepared from tert-butyl (10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamate (29 mg, 0.045 mmol) using acid amine coupling general procedure B and (R)-1-(2-chloro-5-fluorophenyl)-8-nitro-3-oxo-1,2,3,4-tetrahydropyrrolo[1,2-a]pyrazine-6-carboxylic acid (20 mg, 0.057 mmol). Purification by MPLC (CombiFlash, 4 g column, packed with neutral alumina, Gradient Elusion, 4-6% methanol in DCM) provided (R)-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-8-nitro-3-oxo-1,2,3,4-tetrahydropyrrolo[1,2-a]pyrazine-6-carboxamide (50 mg, 95% yield) as an off-white solid. LC-MS m/z 935.25 [M+H]⁺.

### Step 3. (R)-8-amino-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-3-oxo-1,2,3,4-tetrahydropyrrolo[1,2-a]pyrazine-6-carboxamide

To a solution of (R)-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-8-nitro-3-oxo-1,2,3,4-tetrahydropyrrolo[1,2-a]pyrazine-6-carboxamide (50 mg, 32 μmol) in ethanol (2.0 mL) were added iron (18 mg, 0.32 mmol) and saturated ammonium chloride in water (0.5 mL). The solution was heated to 70° C. and stirred for 2 hours. The mixture was filtered through celite bed at hot condition, celite bed was washed with excess methanol in DCM (1:9). The combined filtrate was concentrated under reduced pressure to get the crude material as brown sticky solid. The crude material was dissolved in DCM and washed with H₂O, then brine, dried over Na₂SO₄, filtered, and concentrated under reduced pressure to give crude (R)-8-amino-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-3-oxo-1,2,3,4-tetrahydropyrrolo[1,2-a]pyrazine-6-carboxamide (30 mg) as a brown solid, which was taken for further transformations without additional purifications. LC-MS m/z 903.25 [M−H]⁺, Rt=0.78 min.

### Step 4. N-((R)-1-(2-chloro-5-fluorophenyl)-6-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamoyl)-3-oxo-1,2,3,4-tetrahydropyrrolo[1,2-a]pyrazin-8-yl)benzo[d]isothiazole-3-carboxamide

N-((R)-1-(2-chloro-5-fluorophenyl)-6-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamoyl)-3-oxo-1,2,3,4-tetrahydropyrrolo[1,2-a]pyrazin-8-yl)benzo[d]isothiazole-3-carboxamide was prepared from (R)-1-(2-chloro-5-fluorophenyl)-N-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)-8-nitro-3-oxo-1,2,3,4-tetrahydropyrrolo[1,2-a]pyrazine-6-carboxamide (30 mg, 0.033 mmol) using acid amine coupling general procedure A and benzo[d]isothiazole-3-carboxylic acid (5.9 mg, 0.033 mmol). Purification with reversed-phase HPLC (Method N) provided N-((R)-1-(2-chloro-5-fluorophenyl)-6-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamoyl)-3-oxo-1,2,3,4-tetrahydropyrrolo[1,2-a]pyrazin-8-yl)benzo[d]isothiazole-3-carboxamide (7.6 mg, 20% yield) as an off-white solid. LC-MS m/z 1067.80 [M+H]⁺, Rt=1.53 min, HPLC: 93.81%, Rt=7.15 min, Chiral HPLC: 96.80%, Rt=12.67 min. ¹H NMR (400 MHz, DMSO-D₆) δ=9.82 (s, 1H), 8.98 (s, 1H), 8.88 (d, J=2.5 Hz, 1H), 8.63 (d, J=8.1 Hz, 1H), 8.56 (t, J=5.9 Hz, 1H), 8.30 (d, J=8.2 Hz, 1H), 8.12 (t, J=5.6 Hz, 1H), 7.83 (d, J=9.4 Hz, 1H), 7.69-7.62 (m, 1H), 7.60-7.55 (m, 1H), 7.42-7.36 (m, 4H), 7.32-7.29 (m, 1H), 7.16 (dd, J=3.1, 9.1 Hz, 1H), 7.07-7.01 (m, 2H), 6.22 (d, J=1.2 Hz, 1H), 5.12 (br s, 1H), 5.06 (s, 2H), 4.54 (d, J=9.4 Hz, 1H), 4.47-4.39 (m, 2H), 4.34 (d, J=1.2 Hz, 1H), 4.25-4.19 (m, 1H), 3.65 (d, J=4.8 Hz, 1H), 3.20-3.17 (m, 2H), 2.44 (s, 3H), 2.25 (dd, J=7.2, 14.2 Hz, 1H), 2.14-2.06 (m, 1H), 2.03-1.97 (m, 2H), 1.93-1.85 (m, 1H), 1.52-1.43 (m, 4H), 1.28-1.24 (m, 10H), 0.93 (s, 9H) ppm.

### Example 15

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate warmed to room temperature. PIK3CA enzyme (e.g. 1H1047R, E542K, E545K, or wild-type) (1 μL of 2 nM solution in Enzyme Assay Buffer (comprising 50 mM HEPES pH 7.4, 50 mM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 nM HEPES pH7.4, 50 mM NaCl, 5 mM DTT and 0.03% CHAPS) to start the reaction, and the plate was shaken for 10 seconds, then spun briefly at 1500 rpm, and then incubated for 60 minutes at room temperature. The reaction was stopped by adding 2 μL of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H1047R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 μM are represented as “D”; and compounds having an IC₅₀ greater than 10 μM but less than or equal to 100 μM are represented as “E”.

### Example 16

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/mL hydrocortisone, 100 ng/mL Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT S473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 μM but less than or equal to 5 μM are represented as “B”; compounds having an IC₅₀ greater than 5 μM but less than or equal to 10 μM are represented as “C” compounds having an IC₅₀ greater than 10 μM but less than or equal to 36 μM are represented as “D”; and compounds having an IC₅₀ greater than 36 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound of formula I: 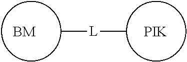
or a pharmaceutically acceptable salt thereof, wherein:
PIK is a first PI3K binding moiety capable of binding to PI3Kα;
L is a bivalent moiety that connects PIK to BM; and
BM is a binding motif LBM, PIK2, or T, wherein:
LBM is an E3 ubiquitin ligase binding moiety;
PIK2 is a second PI3K binding moiety capable of binding to PI3Kα;
T is R^{A*} or R^{B*} substituted by t instances of R^{TC};
R^{A*} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
R^{B*} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{TC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
t is 0, 1, 2, 3, 4, or 5.

2. The compound of claim 1, wherein the compound is a compound of formula I-a: 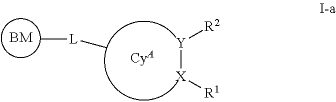
or a pharmaceutically acceptable salt thereof, wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA}; or
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloallylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyAA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, r¹, r², r³, r⁴, r⁵, and r⁶ is independently 0, 1, 2, 3, 4, or 5.

3. The compound of claim 2, wherein the compound is a compound of formula I-a1, I-a2, I-a3, I-a4, I-a5, I-a6, I-a7, I-a8, I-a9, I-a10, I-a11, I-a12, I-a13, I-a14, I-a15, I-a16, I-a17, I-a18, I-a19, I-a20, I-a21, I-a22, I-a23, I-a24, or I-a25: 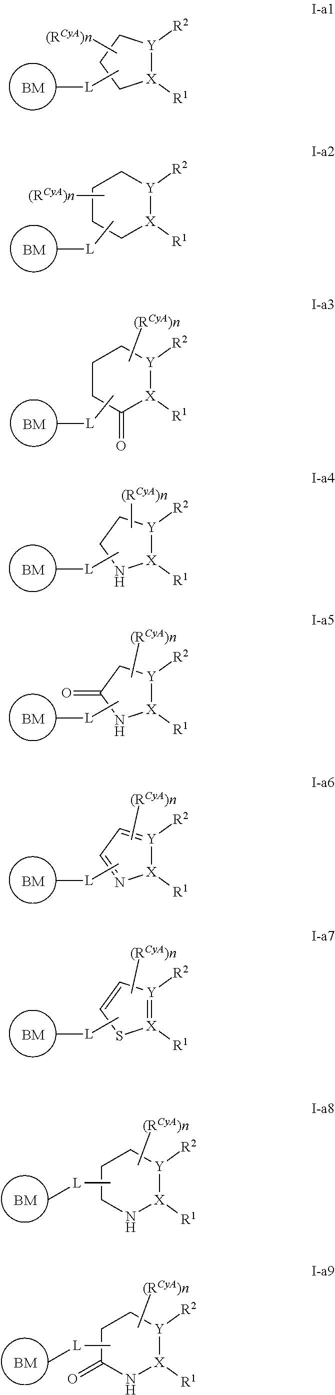 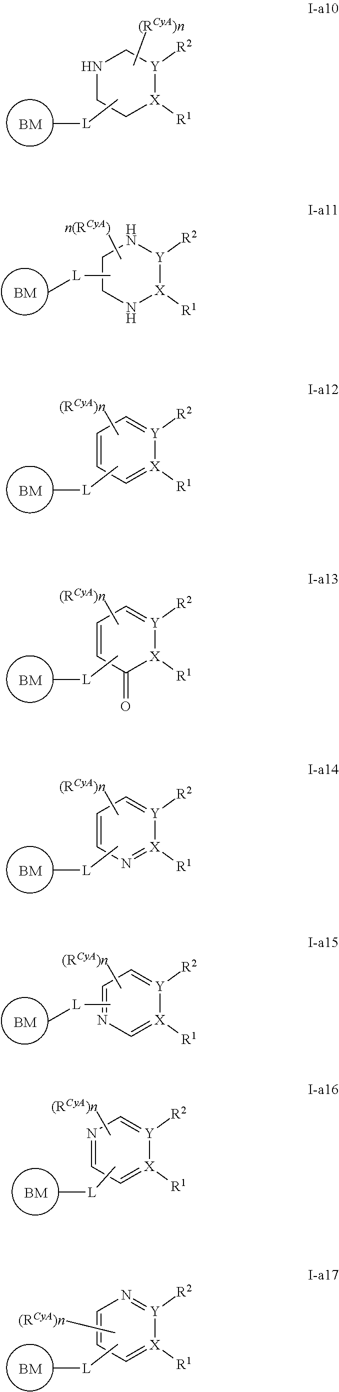 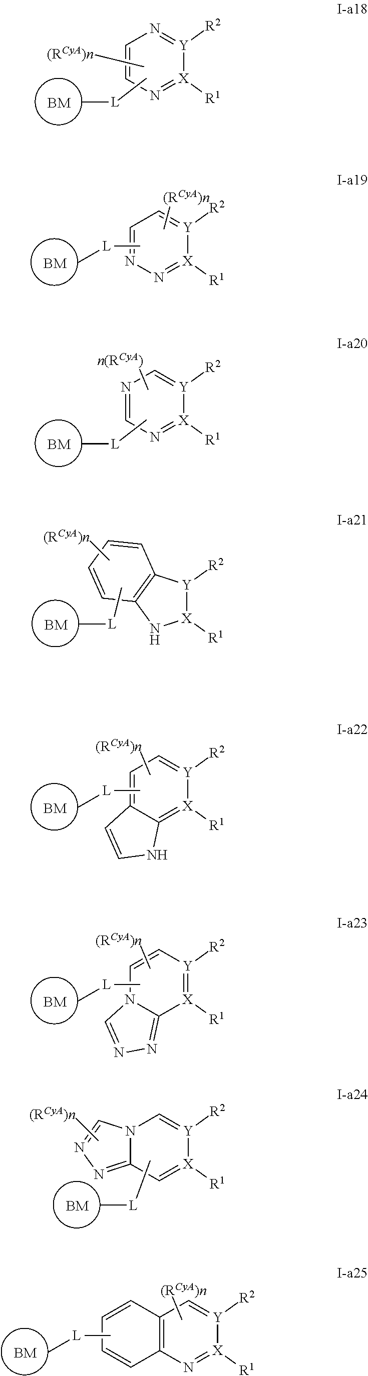
or a pharmaceutically acceptable salt thereof.

4. The compound of claim 2 or 3, wherein Y is C.

5. The compound of any one of claims 2-4, wherein X is C.

6. The compound of any one of claims 2-5, wherein the compound is a compound of formula I-aa1, I-aa2, I-aa3, I-aa4, I-aa4, I-aa5, I-aa6, I-aa7, I-aa8, I-aa9, I-aa10, I-aa11, or I-aa12: 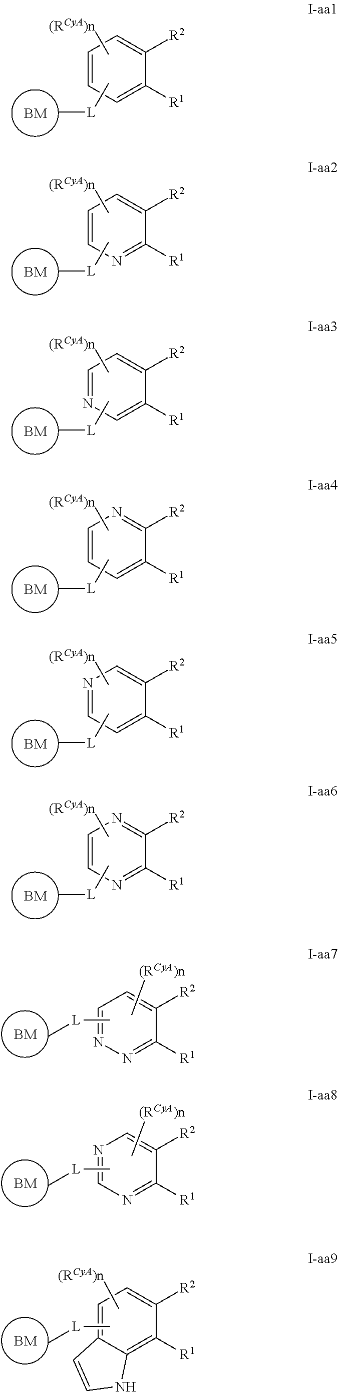 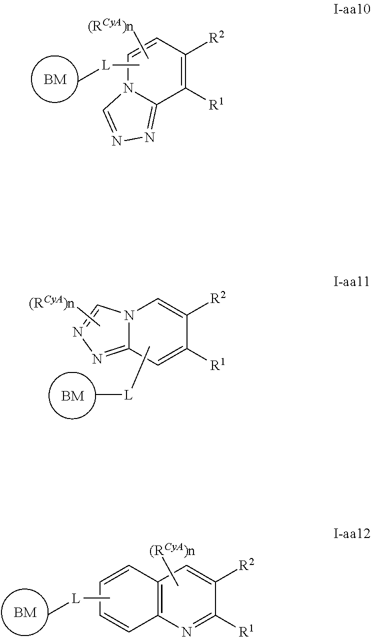
or a pharmaceutically acceptable salt thereof.

7. The compound of claim 2 or 3, wherein Y is CH.

8. The compound of claim 2, 3, or 7, wherein X is CH.

9. The compound of claim 2, 3, or 7, wherein X is N.

10. The compound of any one of claims 2-6, wherein the compound is a compound of formula I-aaa1, I-aaa2, and I-aaa3: 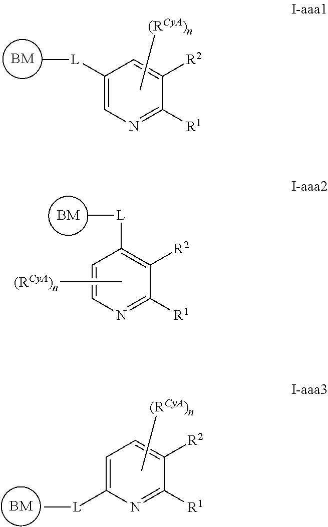
or a pharmaceutically acceptable salt thereof.

11. The compound of any one of claims 2-10, wherein L¹ is —N(H)—.

12. The compound of any one of claims 2-11, wherein R^{1A} is R^{B} substituted by r instances of R^{1C}.

13. The compound of any one of claims 2-12, wherein R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

14. The compound of any one of claims 2-13, wherein R^{1A} is phenyl substituted by r¹ instances of R^{1C}.

15. The compound of any one of claims 2-14, wherein R^{1A} is

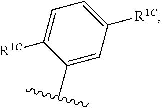

16. The compound of any one of claims 2-15, wherein each instance of R^{1C} is independently halogen, —CN, —O—(C₁₋₆ aliphatic), or C₁₋₆ aliphatic; wherein each C₁₋₆ aliphatic is optionally substituted with one or more halogen atoms.

17. The compound of any one of claims 2-16, wherein each instance of R^{1C} is independently halogen or C₁₋₃ aliphatic optionally substituted with 1-3 halogen.

18. The compound of any one of claims 2-17, wherein R² is —N(H)C(O)—R^{2A}, —N(H)C(O)N(H)—R^{2A}, —C(O)N(H)—R^{2A}, —N(H)—R^{2A}, —S(O)₂CH₂—R^{2A}, —CH₂S(O)₂—R^{2A}, or —C(H)(CH₃)OH.

19. The compound of any one of claims 2-18, wherein R² is —N(H)C(O)—R^{2A}.

20. The compound of any one of claims 2-19, wherein R^{2A} is R^{B} substituted by r² instances of R^{2C}.

21. The compound of any one of claims 2-20, wherein R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

22. The compound of any one of claims 2-19, wherein R^{2A} is

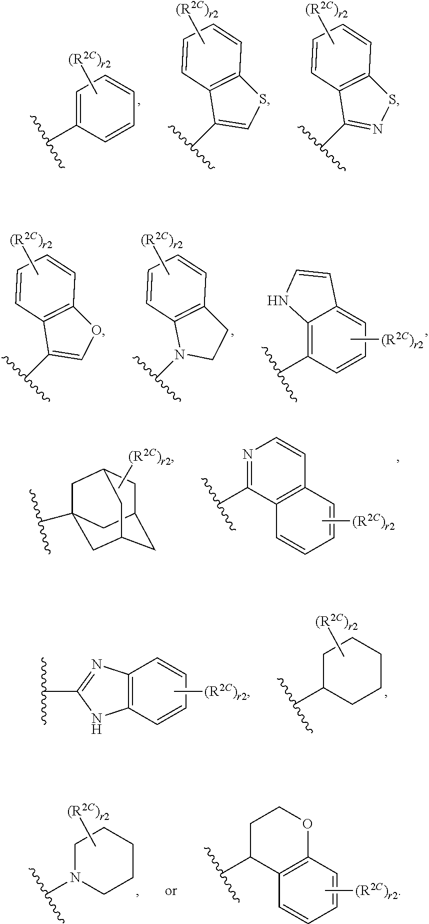

23. The compound of any one of claims 2-17, wherein R² is

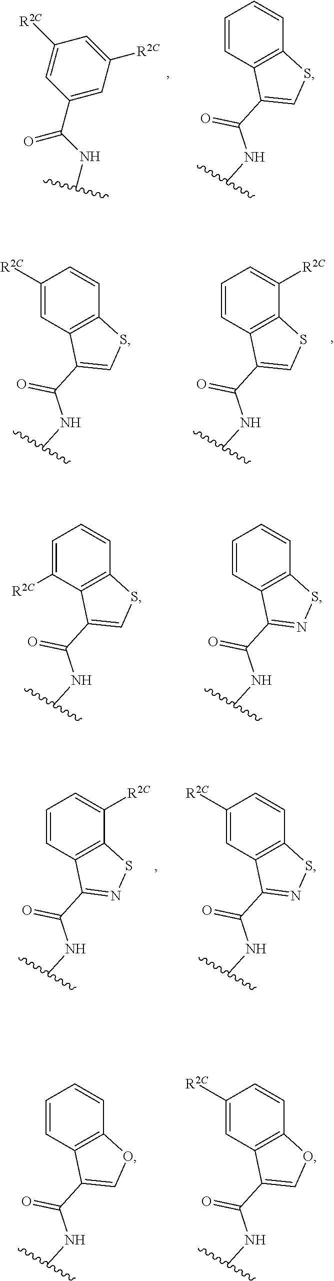

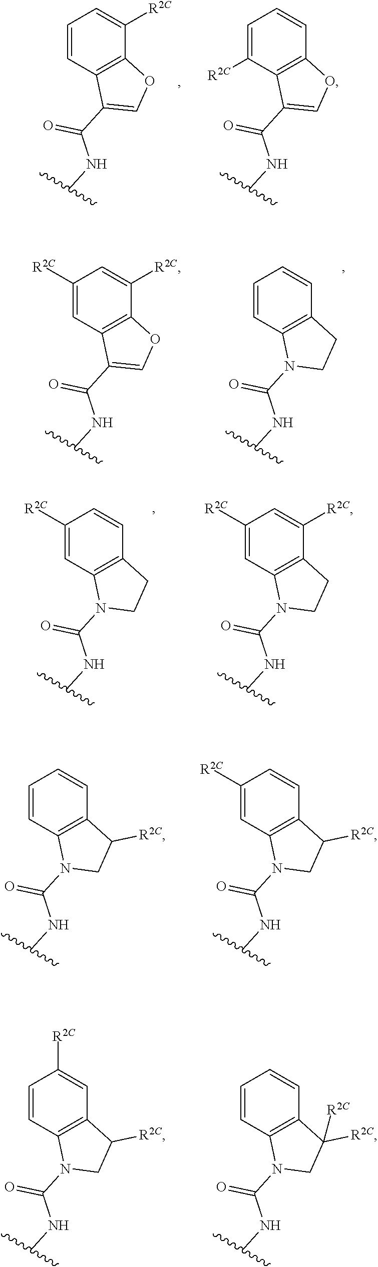

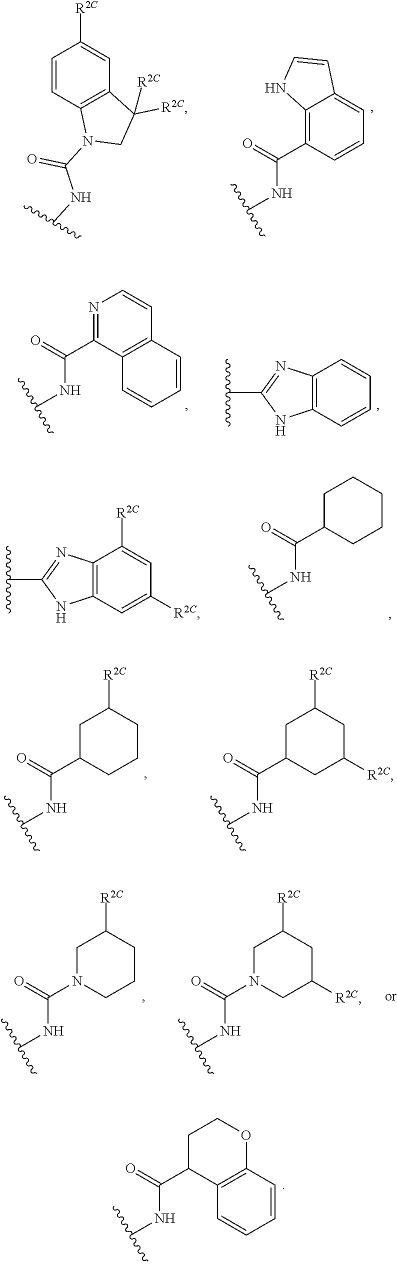

24. The compound of any one of claims 2-23, wherein each instance of R^{2C} is independently halogen, —CN, —O—(C₁₋₆ aliphatic), or C₁₋₆ aliphatic; wherein each C₁₋₆ aliphatic is optionally substituted with one or more halogen atoms.

25. The compound of any one of claims 2-24, wherein each instance of R^{2C} is independently halogen or C₁₋₃ aliphatic optionally substituted with 1-3 halogen.

26. The compound of any one of claims 2-25, wherein each instance of R^{CyA} is independently —C(O)N(H)—R^{CyAA}, —C(O)N(H)CH₂—R^{CyAA}, or —R^{CyAA}.

27. The compound of any one of claims 2-25, wherein each instance of R^{CyA} is independently

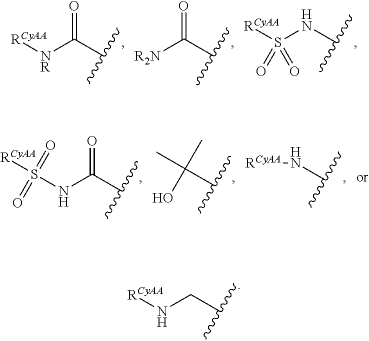

28. The compound of any one of claims 2-27, wherein each instance of R^{CyAA} is independently a 5-6 membered monocyclic heteroaryl ring having 1-4 heteroatoms independently selected from nitrogen, oxygen, and sulfur; wherein said ring is substituted by r⁶ instances of R^{CyAC}.

29. The compound of any one of claims 2-27, wherein each instance of R^{CyAA} is independently a 3-7 membered saturated or partially unsaturated monocyclic heterocyclic ring having 1-2 heteroatoms independently selected from nitrogen, oxygen, and sulfur; wherein said ring is substituted by r⁶ instances of R^{CyAC}.

30. The compound of any one of claims 2-27, wherein each instance of R^{CyAA} is independently

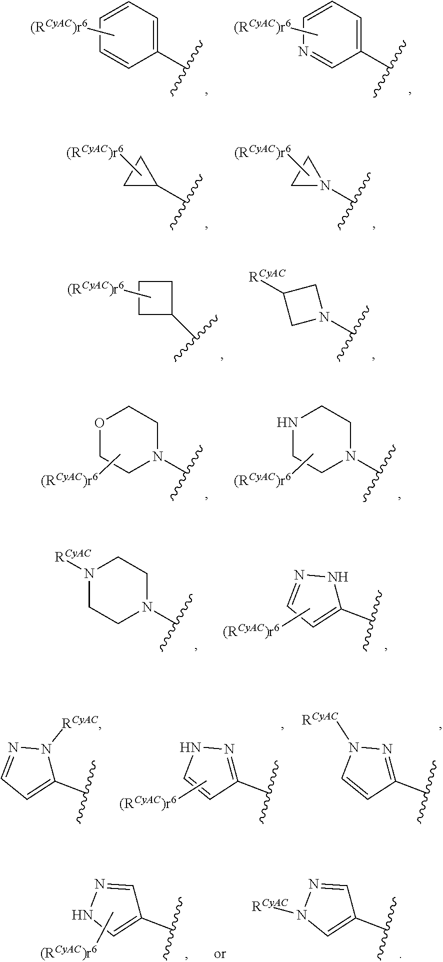

31. The compound of any one of claims 2-27, wherein each instance of R^{CyAA} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium.

32. The compound of any one of claims 2-31, wherein each instance of R^{CyAC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms.

33. The compound of claim 1, wherein the compound is a compound of formula I-b: 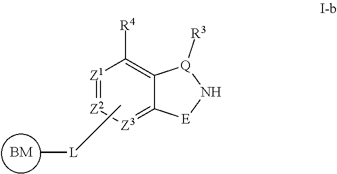
or a pharmaceutically acceptable salt thereof, wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
Q is CH, C(R^{Q}), or N;
Z¹ is CH, C(R^{Z1}), or N;
Z² is CH, C(R^{Z2}), or N;
Z³ is CH, C(R^{Z3}), or N;
R³ is -L³-R^{3A};
R⁴ is -L⁴-R^{4A};
each instance of R^{E} is independently H or -L^{E}-R^{EA};
R^{Q} is -L^{Q}-R^{QA};
R^{Z1} is -L^{Z1}-R^{Z1A};
R^{Z2} is -L^{Z2}-R^{Z2A};
R^{Z3} is -L^{Z3}-R^{Z3A}; or
two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n¹ instances of R^{EEC};
R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p¹ instances of R^{Q3C};
R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C};
each of L³, L⁴, L^{E}, L^{Q}, L^{Z1}, L^{Z2}, and L^{Z3} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L1})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{3A} is R^{C} or R^{D} substituted by s¹ instances of R^{3C};
R^{4A} is R^{C} or R^{D} substituted by s² instances of R^{4C};
R^{EA} is R^{C} or R^{D} substituted by s³ instances of R^{EC};
R^{QA} is R^{C} or R^{D} substituted by s⁴ instances of R^{QC};
R^{Z1A} is R^{C} or R^{D} substituted by s⁵ instances of R^{Z1C};
R^{Z2A} is R^{C} or R^{D} substituted by s⁶ instances of R^{Z2C};
R^{Z3A} is R^{C} or R^{D} substituted by s⁷ instances of R^{Z3C};
R^{L1} is R^{C} or R^{D} substituted by s⁸ instances of R^{L1C};
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{D} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{3C}, R^{4C}, R^{EC}, R^{QC}, R^{Z1C}, R^{Z2C}, R^{Z3C}, R^{L1C}, R^{EEC}, R^{Q3C}, and R^{Z2Z3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n¹, p¹, q¹, s¹, s², s³, s⁴, s⁵, s⁶, s⁷, and s⁸ is independently 0, 1, 2, 3, 4, or 5.

34. The compound of claim 33, wherein the compound is a compound of formula I-b2: 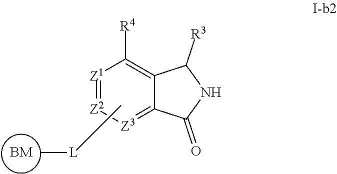
or a pharmaceutically acceptable salt thereof

35. The compound of claim 33, wherein the compound is a compound of formula I-b3: 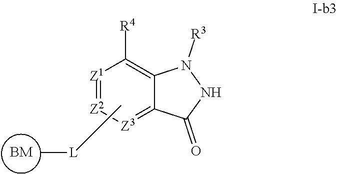
or a pharmaceutically acceptable salt thereof.

36. The compound of claim 33, wherein the compound is a compound of formula I-b4, I-b5, or I-b6: 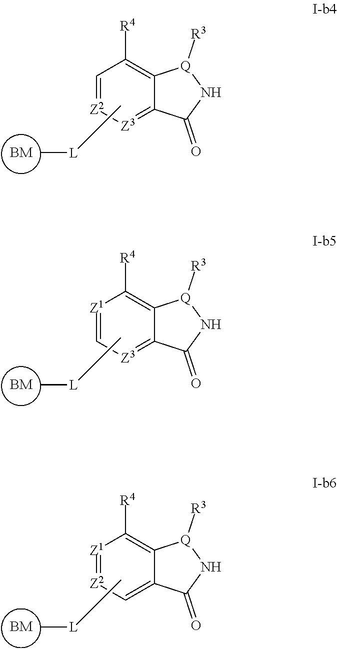
or a pharmaceutically acceptable salt thereof.

37. The compound of claim 33, wherein the compound is a compound of I-b7, I-b8, or I-b9: 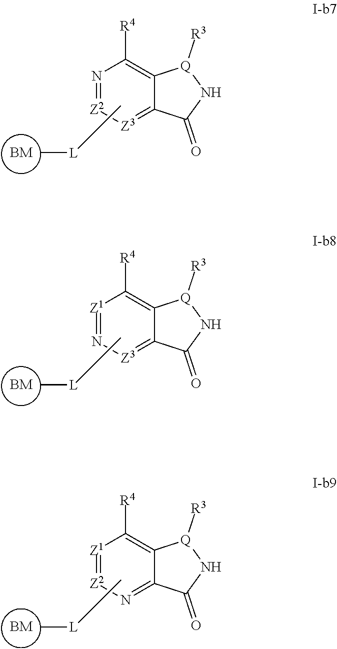
or a pharmaceutically acceptable salt thereof.

38. The compound of claim 33, wherein the compound is a compound of formula I-b10, I-b11, or I-b12: 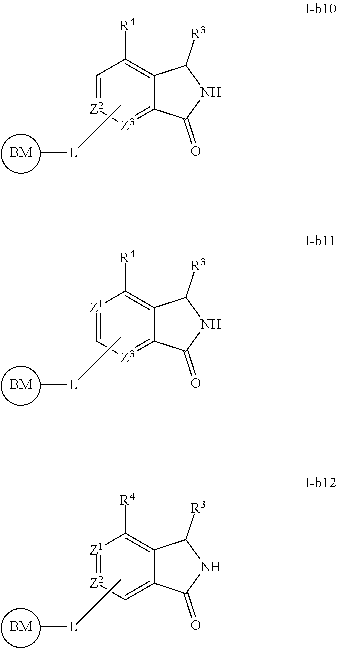
or a pharmaceutically acceptable salt thereof.

39. The compound of claim 33-38, wherein Z¹ is CH.

40. The compound of claim 33, wherein the compound is a compound of formula I-bb3 or I-bbb5: 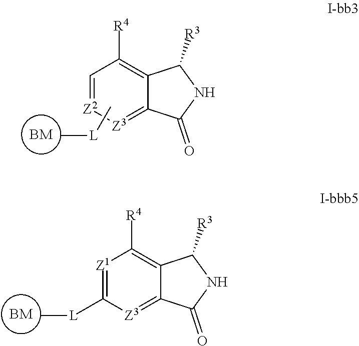
or a pharmaceutically acceptable salt thereof.

41. The compound of any one of claims 33-40, wherein Z³ is CH or N.

42. The compound of any one of claims 33-41, wherein L³ is a covalent bond.

43. The compound of any one of claims 33-42, wherein R^{3A} is R^{D} substituted by s¹ instances of R^{3C}.

44. The compound of any one of claims 33-43, wherein R^{3A} is phenyl or an 8-10 membered bicyclic heteroaryl ring having 1-4 heteroatoms independently selected from nitrogen, oxygen, and sulfur, wherein R^{3A} is substituted by s¹ instances of R^{3C}.

45. The compound of any one of claims 33-44, wherein R^{3A} is phenyl substituted by s¹ instances of R^{3C}.

46. The compound of any one of claims 33-45, wherein R³ is

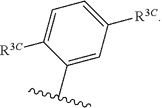

47. The compound of any one of claims 33-46, wherein each instance of R^{3C} is independently halogen, —CN, —O—(C₁₋₆ aliphatic), or C₁₋₆ aliphatic; wherein each C₁₋₆ aliphatic is optionally substituted with one or more halogen atoms.

48. The compound of any one of claims 33-47, wherein each instance of R^{3C} is independently halogen or C₁₋₃ aliphatic optionally substituted with 1-3 halogen.

49. The compound of any one of claims 33-48, wherein R⁴ is —N(H)C(O)—R^{4A}, —N(H)C(O)N(H)—R^{4A}, —C(O)N(H)—R^{4A}, —N(H)—R^{4A}, —S(O)₂CH₂—R^{4A}, —CH₂S(O)₂—R^{4A}, or —C(H)(CH₃)OH.

50. The compound of any one of claims 33-49, wherein R⁴ is —N(H)C(O)—R^{4A}.

51. The compound of any one of claims 33-50, wherein R^{4A} is R^{D} substituted by s² instances of R^{4C}.

52. The compound of any one of claims 33-51, wherein R^{4A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{4A} is substituted by s² instances of R^{4C}.

53. The compound of any one of claims 33-50, wherein R^{4A} is

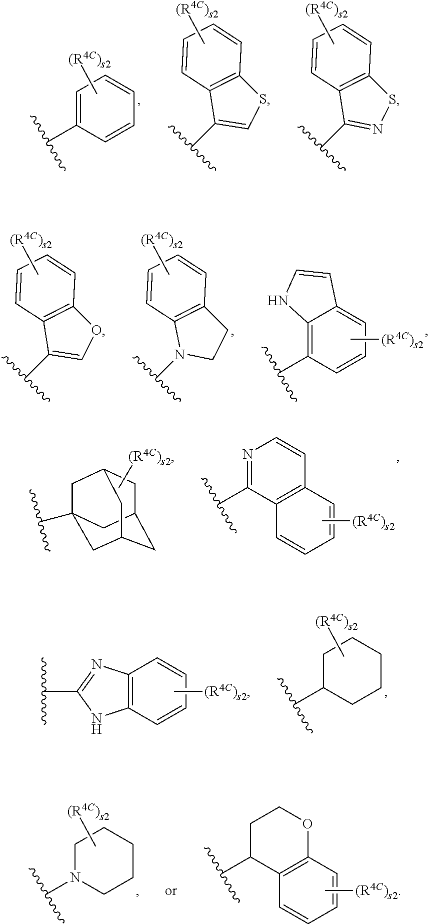

54. The compound of any one of claims 33-48, wherein R⁴ is

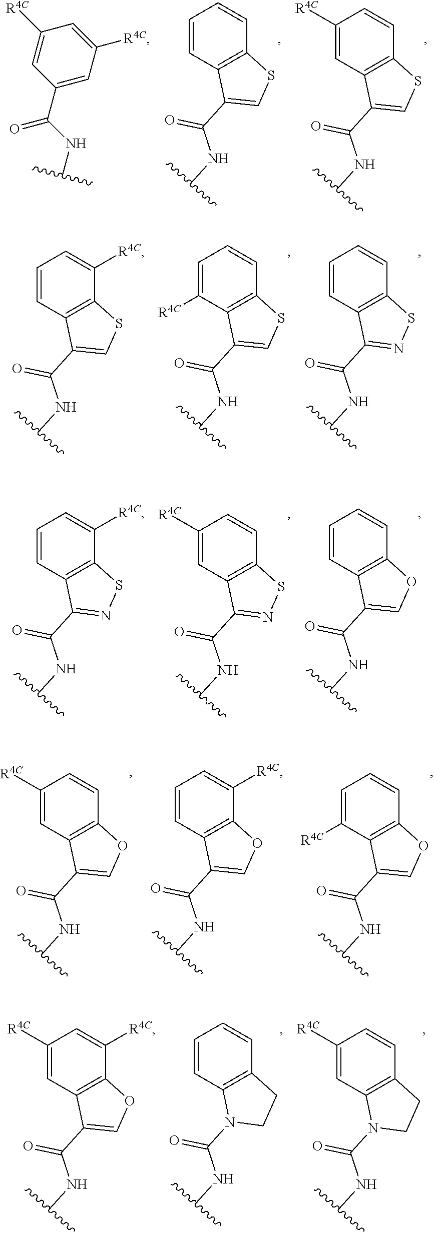

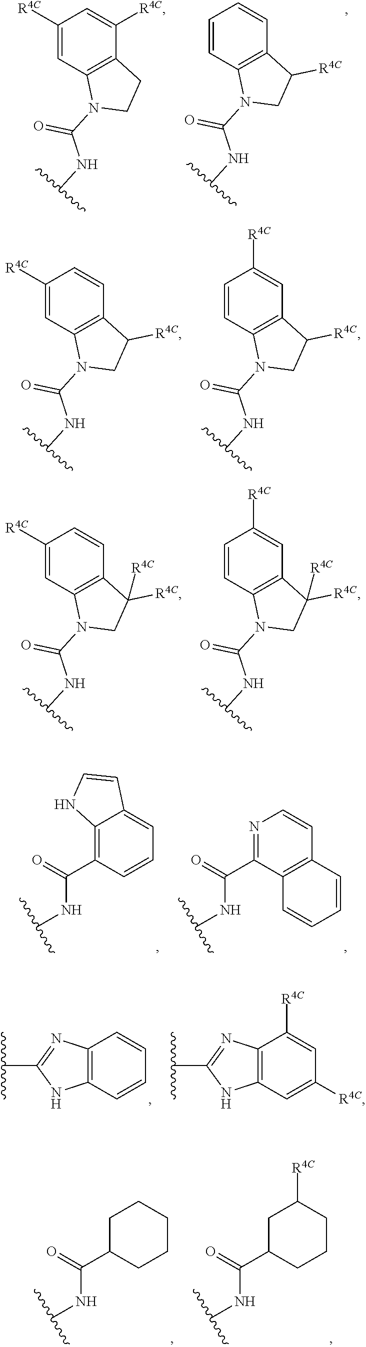

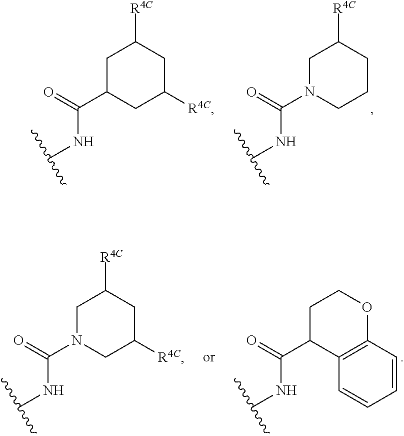

55. The compound of any one of claims 33-54, wherein each instance of R^{4C} is independently halogen, —CN, —O—(C₁₋₆ aliphatic), or C₁₋₆ aliphatic; wherein each C₁₋₆ aliphatic is optionally substituted with one or more halogen atoms.

56. The compound of any one of claims 33-55, wherein each instance of R^{4C} is independently halogen or C₁₋₃ aliphatic optionally substituted with 1-3 halogen.

57. The compound of any one of claims 33-56, wherein Z² is C(R^{Z2A}).

58. The compound of any one of claims 33-57, wherein R^{Z2A} is a C₁₋₄ aliphatic chain; a 3-5 membered saturated or partially unsaturated monocyclic carbocyclic ring; or a 3-5 membered saturated or partially unsaturated monocyclic heterocyclic ring having 1-2 heteroatoms independently selected from nitrogen, oxygen, and sulfur; wherein R^{Z2A} is substituted by s⁶ instances of a group independently selected from halogen, —CN, —OH, —O-(optionally substituted C₁₋₃ aliphatic), and an optionally substituted C₁₋₃ aliphatic.

59. The compound of any one of claims 33-57, wherein R^{Z2A} is an 8-10 membered bicyclic heteroaryl ring having 1-4 heteroatoms independently selected from nitrogen, oxygen, and sulfur; wherein said ring is substituted by s⁶ instances of R^{Z2C}.

60. The compound of any one of claims 33-57, wherein R^{Z2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by s⁶ instances of R^{Z2C}.

61. The compound of any one of claims 33-57, wherein R^{Z2A} is

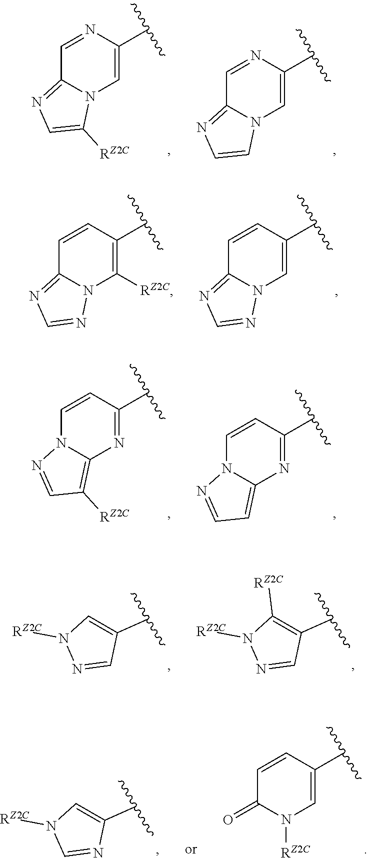

62. The compound of any one of claims 33-57 or 59-61, wherein each instance of R^{Z2C} is independently oxo, halogen, —CN, —OH, —O—(C₁₋₃ aliphatic), or C₁₋₃ aliphatic, wherein each C₁₋₃ aliphatic is optionally substituted with one or more halogen atoms.

63. The compound of claim 1, wherein the compound is a compound of formula I-c: 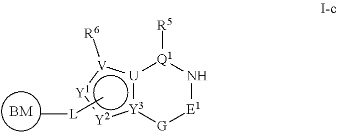
or a pharmaceutically acceptable salt thereof, wherein:
E¹ is —C(O)—, —C(R^{E1})₂—, —C(R^{E1})₂C(R^{E1})₂—, —C(S)—, —S(O)₂—, —OC(O)—, —N(R^{E1})C(O)—, —C(O)N(R^{E1})—, or —C(R^{E1})₂C(O)—;
G is CH₂, CH(R^{G}), C(R^{G})₂, or a covalent bond;
Q¹ is CH, C(R^{Q1}), or N;
Y¹ is CH, C(R^{Y1}), or N;
Y² is CH, C(R^{Y2}), N, or N(R^{Y2});
Y³ is C or N;
U is C or N;
V is C or N; provided that at least one of Y¹, Y², Y³, U, and V is N;
R⁵ is -L⁵-R^{5A};
R⁶ is -L⁶-R^{6A};
each instance of R^{E1} is independently H or -L^{E1}-R^{E1A};
each instance of R^{G} is independently -L^{G}-R^{GA};
R^{Q1} is -L^{Q1}-R^{Q1A};
R^{Y1} is -L^{Y1}-R^{Y1A};
R^{Y2} is -L^{Y2-}R^{Y2A}; or
two instances of R^{E1} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n² instances of R^{E1EC};
R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p² instances of R^{Q5C};
each of L⁵, L⁶, L^{E1}, L^{G}, L^{Q1}, L^{Y1}, and L^{Y2} is independently a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{5A} is R^{F} or R^{H} substituted by v¹ instances of R^{5C};
R^{6A} is R^{F} or R^{H} substituted by v² instances of R^{6C};
each instance of R^{E1A} is independently R^{F} or R^{H} substituted by v³ instances of R^{EIC};
each instance of R^{GA} is independently R^{F} or R^{H} substituted by v⁴ instances of R^{GC};
R^{Q1A} is R^{F} or R^{H} substituted by v⁵ instances of R^{Q1C};
R^{Y1A} is R^{F} or R^{H} substituted by v⁶ instances of R^{Y1C};
R^{Y2A} is R^{F} or R^{H} substituted by v⁷ instances of R^{Y2C};
R^{L2} is R^{F} or R^{H} substituted by v⁸ instances of R^{L2C};
each instance of R^{F} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{H} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{5C}, R^{6C}, R^{E1C}, R^{GC}, R^{Q1C}, R^{Y1C}, R^{Y2C}, R^{L2C}, R^{E1EC}, and R^{Q5C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n², p², v¹, v², v³, v⁴, v⁵, v⁶, v⁷, and v⁸ is independently 0, 1, 2, 3, 4, or 5.

64. The compound of claim 63, wherein the compound is a compound of formula I-c1: 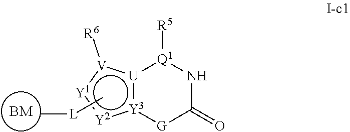
or a pharmaceutically acceptable salt thereof.

65. The compound of claim 63 or 64, wherein the compound is a compound of formula I-c2: 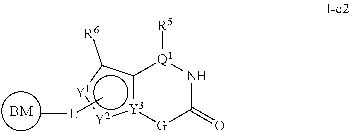
or a pharmaceutically acceptable salt thereof.

66. The compound of claim 63 or 64, wherein the compound is a compound of formula I-c3, I-c4, or I-c5: 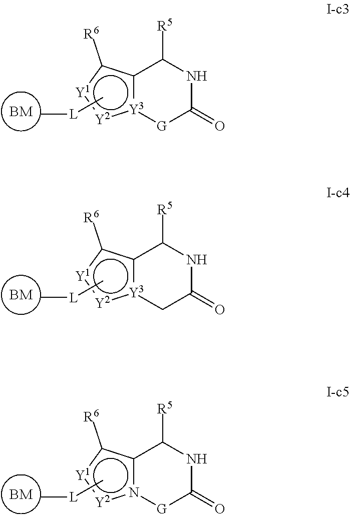
or a pharmaceutically acceptable salt thereof.

67. The compound of claim 63 or 64, wherein the compound is a compound of formula I-c6 or I-c7: 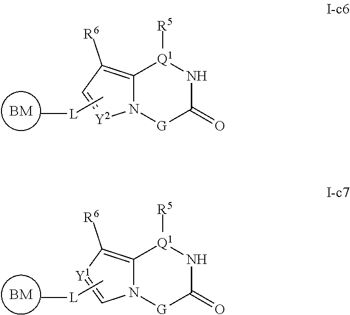
or a pharmaceutically acceptable salt thereof.

68. The compound of claim 63 or 64, wherein the compound is a compound of formula I-cc1 or I-cc2: 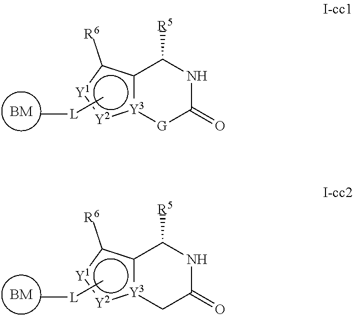
or a pharmaceutically acceptable salt thereof.

69. The compound of claim 63 or 64, wherein the compound is a compound of formula I-ccc1, I-ccc3, or I-ccc5: 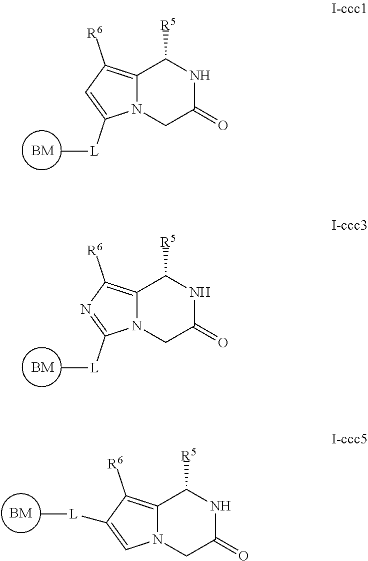
or a pharmaceutically acceptable salt thereof.

70. The compound of any one of claims 63-69, wherein Y¹ is CH.

71. The compound of any one of claims 63-69, wherein Y¹ is N.

72. The compound of any one of claims 63-71, wherein Y³ is N.

73. The compound of any one of claims 63-72, wherein L⁵ is a covalent bond.

74. The compound of any one of claims 63-73, wherein R^{5A} is R^{H} substituted by v¹ instances of R^{5C}.

75. The compound of any one of claims 63-74, wherein R^{5A} is phenyl or an 8-10 membered bicyclic heteroaryl ring having 1-4 heteroatoms independently selected from nitrogen, oxygen, and sulfur, wherein R^{5A} is substituted by v¹ instances of R^{5C}.

76. The compound of any one of claims 63-75, wherein R^{5A} is phenyl substituted by v¹ instances of R^{5C}.

77. The compound of any one of claims 63-76, wherein R⁵ is

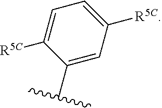

78. The compound of any one of claims 63-77, wherein each instance of R^{5C} is independently halogen, —CN, —O—(C₁₋₆ aliphatic), or C₁₋₆ aliphatic; wherein each C₁₋₆ aliphatic is optionally substituted with one or more halogen atoms.

79. The compound of any one of claims 63-78, wherein each instance of R^{5C} is independently halogen or C₁₋₃ aliphatic optionally substituted with 1-3 halogen.

80. The compound of any one of claims 63-79, wherein R⁶ is —N(H)C(O)—R^{6A}, —N(H)C(O)N(H)—R^{6A}, —C(O)N(H)—R^{6A}, —N(H)—R^{6A}, —S(O)₂CH₂—R^{6A}, —CH₂S(O)₂—R^{6A}, or —C(H)(CH₃)OH.

81. The compound of any one of claims 63-80, wherein R⁶ is —N(H)C(O)—R^{6A}.

82. The compound of any one of claims 63-81, wherein R^{6A} is R^{H} substituted by v² instances of R^{86C}.

83. The compound of any one of claims 63-82, wherein R^{6A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{6A} is substituted by v² instances of R^{6C}.

84. The compound of any one of claims 63-81, wherein R^{6A} is

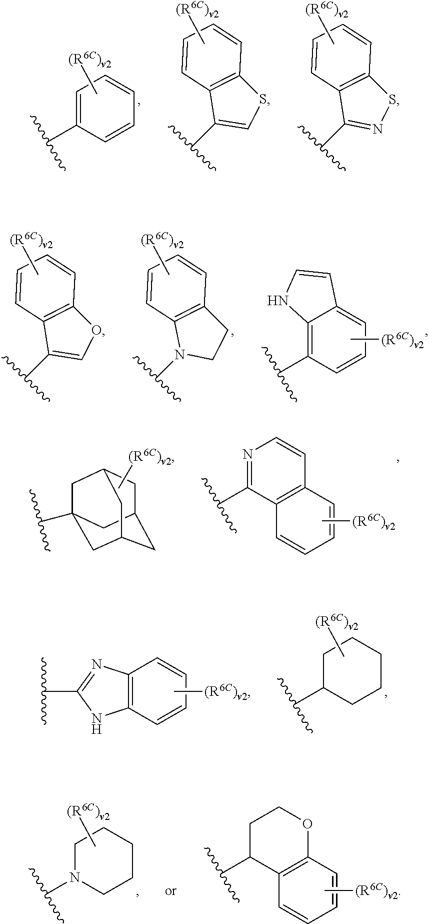

85. The compound of any one of claims 63-79, wherein R⁶ is

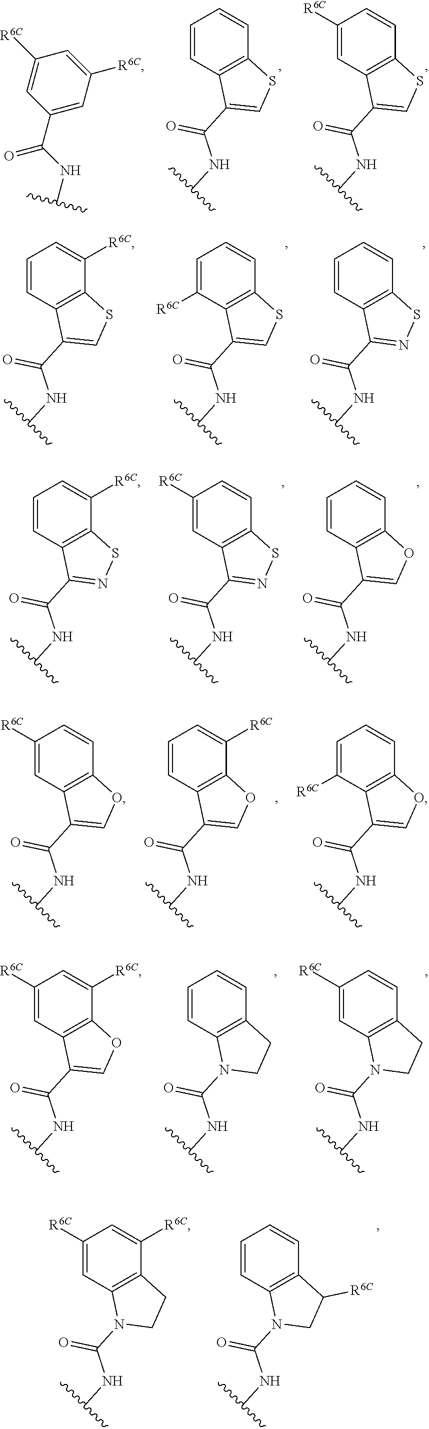

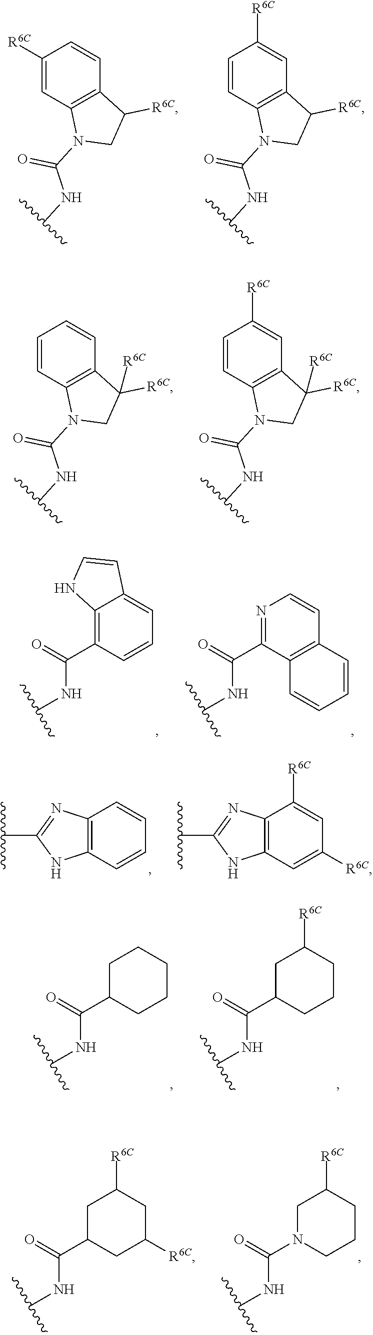

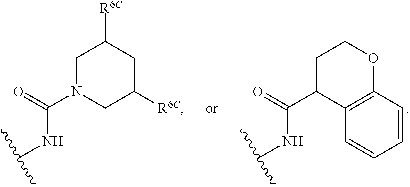

86. The compound of any one of claims 63-85, wherein each instance of R^{6C} is independently halogen, —CN, —O—(C₁₋₆ aliphatic), or C₁₋₆ aliphatic; wherein each C₁₋₆ aliphatic is optionally substituted with one or more halogen atoms.

87. The compound of any one of claims 63-86, wherein each instance of R^{6C} is independently halogen or C₁₋₃ aliphatic optionally substituted with 1-3 halogen.

88. The compound of any one of claims 63-87, wherein Y² is C(R^{Y2}).

89. The compound of any one of claims 63-88, wherein R^{Y2} is —C(O)N(H)—R^{Y2A}, —C(O)N(H)CH₂—R^{Y2A}, or —R^{Y2A}.

90. The compound of any one of claims 63-89, wherein R^{Y2A} is a 5-6 membered monocyclic heteroaryl ring having 1-4 heteroatoms independently selected from nitrogen, oxygen, and sulfur; wherein said ring is substituted by v⁷ instances of R^{Y2C}.

91. The compound of any one of claims 63-89, wherein R^{Y2A} is a 3-7 membered saturated or partially unsaturated monocyclic heterocyclic ring having 1-2 heteroatoms independently selected from nitrogen, oxygen, and sulfur; wherein said ring is substituted by v⁷ instances of R^{Y2C}.

92. The compound of any one of claims 63-89, wherein R^{Y2A} is

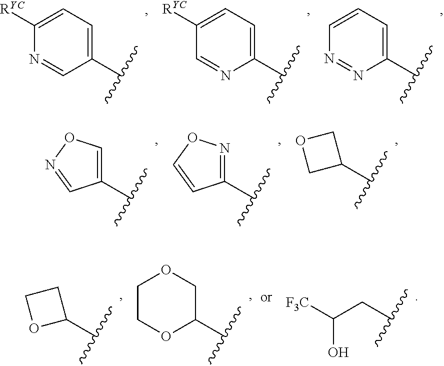

93. The compound of any one of claims 63-89, wherein R^{Y2A} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium.

94. The compound of any one of claims 63-92, wherein each instance of R^{Y2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms.

95. The compound of claim 1, wherein the compound is a compound of formula I-d: 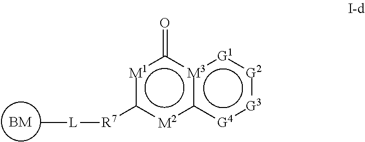
or a pharmaceutically acceptable salt thereof, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-BM;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3})—, —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z⁷ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R_{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

96. The compound of claim 95, wherein G³ is CR⁸.

97. The compound of claim 95, wherein G⁴ is CR⁸.

98. The compound of any one of claims 95-97, wherein the compound is a compound of formula I-d1 or I-d2: 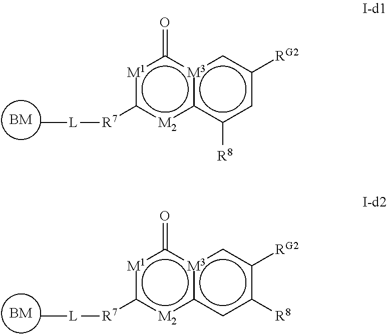
or a pharmaceutically acceptable salt thereof.

99. The compound of any one of claims 95-98, wherein M² is O.

100. The compound of any one of claims 95-99, wherein M³ is C.

101. The compound of any one of claims 95-100, wherein M¹ is CH.

102. The compound of any one of claims 95-101, wherein the compound is a compound of formula I-d3 or I-d4: 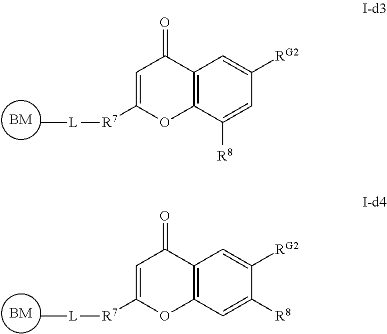
or a pharmaceutically acceptable salt thereof.

103. The compound of any one of claims 95-100, wherein M¹ is C(R^{M1}).

104. The compound of any one of claims 95-100, wherein the compound is a compound of formula I-d5 or I-d6: 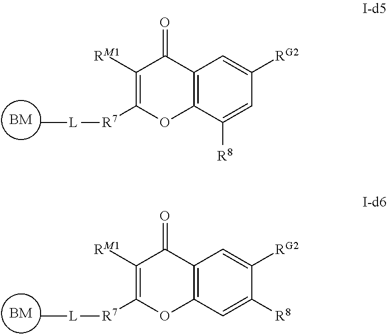
or a pharmaceutically acceptable salt thereof.

105. The compound of any one of claims 95-98, wherein M² is N.

106. The compound of any one of claims 95-98, wherein M³ is N.

107. The compound of any one of claims 95-98, wherein the compound is a compound of formula I-d7 or I-d8: 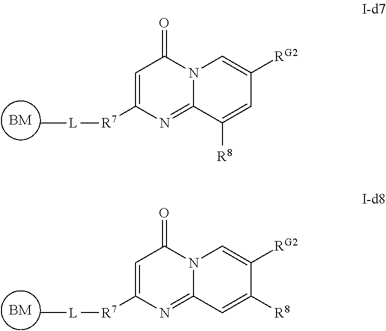
or a pharmaceutically acceptable salt thereof.

108. The compound of any one of claims 95-98, wherein the compound is a compound of formulas I-d9 or I-d10: 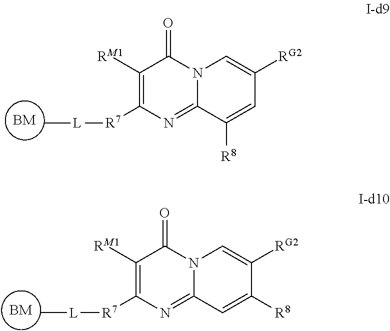
or a pharmaceutically acceptable salt thereof.

109. The compound of any one of claims 95-98, wherein M¹ is N(R^{M1}).

110. The compound of any one of claims 95-98, wherein the compound is a compound of formula I-d11 or I-d12: 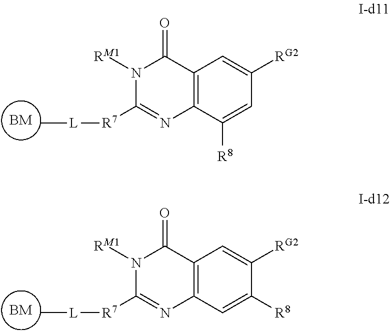
or a pharmaceutically acceptable salt thereof.

111. The compound of any one of claims 95-110, wherein L^{G2} is a covalent bond or —O—.

112. The compound of any one of claims 95-111, wherein R^{G2A} is R^{K} substituted by z⁴ instances of R^{G2C}.

113. The compound of any one of claims 95-111, wherein R^{G2A} is a C₁₋₆ aliphatic chain or halogen.

114. The compound of any one of claims 95-110, wherein R^{G2} is methyl.

115. The compound of any one of claims 95-114, wherein R^{7A} is a 3-7 membered saturated or partially unsaturated monocyclic heterocyclic ring having 1-2 heteroatoms independently selected from nitrogen, oxygen, and sulfur, wherein said ring is substituted by z¹ instances of R^{7C}.

116. The compound of any one of claims 95-114, wherein R^{7A} is 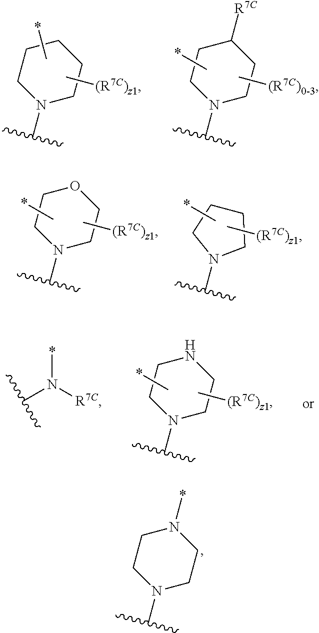
wherein [symbol] represents a covalent bond to L.

117. The compound of any one of claims 95-116, wherein each instance of R^{7C} is independently halogen, —CN, —O—(C₁₋₆ aliphatic), or C₁₋₆ aliphatic; wherein each C₁₋₆ aliphatic is optionally substituted with one or more halogen atoms.

118. The compound of any one of claims 95-117, wherein each instance of R^{7C} is independently halogen or C₁₋₃ aliphatic optionally substituted with 1-3 halogen.

119. The compound of any one of claims 95-118, wherein R⁸ is —CH(CH₃)N(R)—R^{8A}, —CH(R^{L3})N(H)—R^{8A}, —CH(CH₃)N(H)—R^{8A}, or —R^{8A}.

120. The compound of any one of claims 95-118, wherein R⁸ is —CH(CH₃)N(H)—R^{8A}.

121. The compound of any one of claims 95-120, wherein R^{8A} is Cy^{B}-R^{CyB} substituted by z² instances of R^{8C}.

122. The compound of any one of claims 95-121, wherein Cy^{B} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

123. The compound of any one of claims 95-122, wherein R^{8A} is

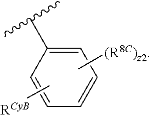

124. The compound of any one of claims 95-123, wherein R⁸ is

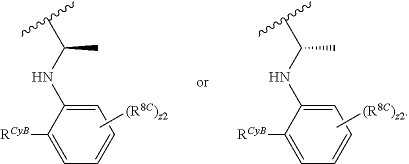

125. The compound of any one of claims 95-124, wherein each instance of R^{8C} is halogen, —OH, —C(O)OR, —C(O)NR₂, —S(O)R, —S(O)₂R, —S(O)NR₂, —S(O)₂NR₂, or an optionally substituted C₁₋₆ aliphatic.

126. The compound of any one of claims 95-125, wherein each instance of R^{8C} is independently —OH, —C(O)OH, —C(O)NH, —S(O)NH₂, or —S(O)₂NH₂.

127. The compound of any one of claims 95-126, wherein R^{M1A} is R^{K} substituted by z⁶ instances of R^{M1C}.

128. The compound of any one of claims 95-127, wherein R^{M1A} is a C₁₋₆ aliphatic chain or phenyl substituted by z⁶ instances of R^{M1C}.

129. The compound of any one of claims 95-128, wherein R^{M1} is methyl.

130. The compound of any one of claims 1-129, wherein BM is LBM.

131. The compound of any one of claims 1-130, wherein LBM is a cereblon (CRBN) E3 ubiquitin ligase binding moiety.

132. The compound of any one of claims 1-131, wherein LBM is

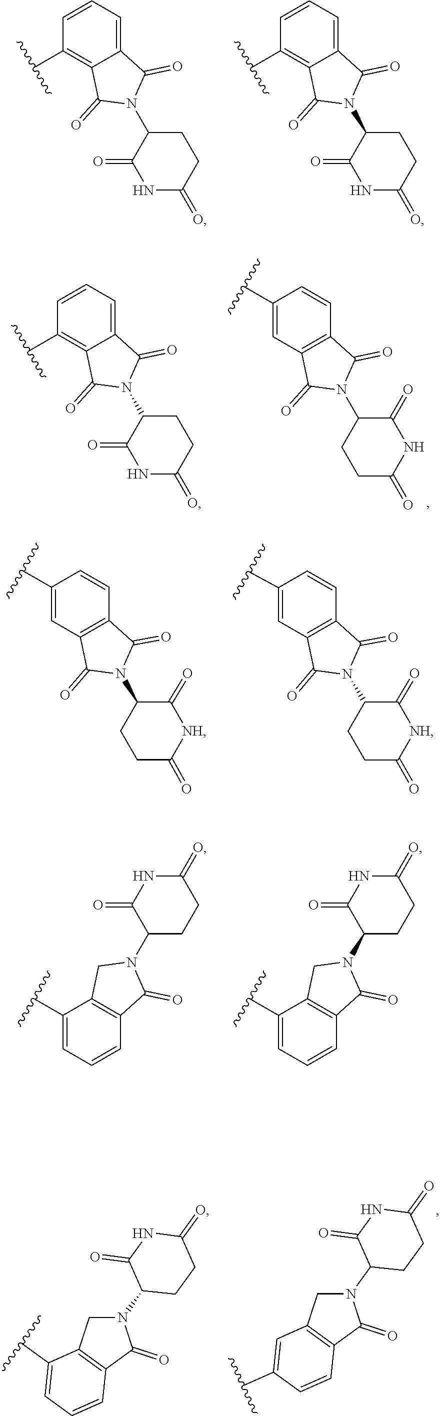

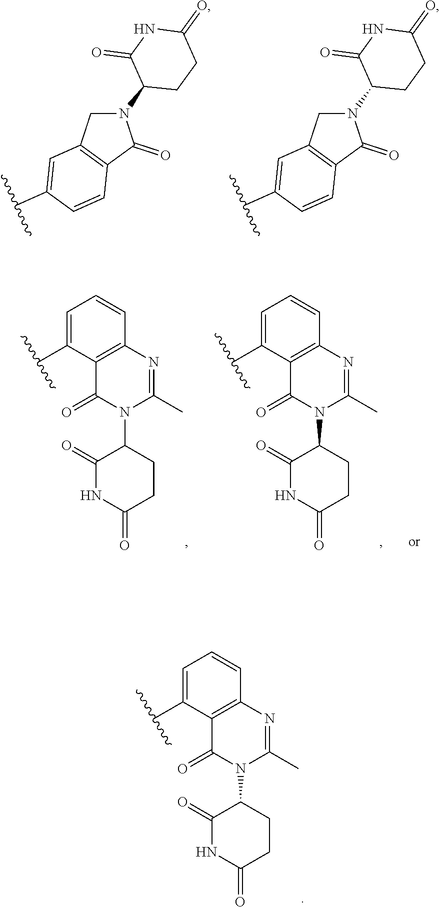 [SMILES: *c1ccc2c(c1)CN([C@@H]1CCC(=O)NC1=O)C2=O ; *c1ccc2c(c1)CN([C@H]1CCC(=O)NC1=O)C2=O ; *c1cccc2nc(C)n(C3CCC(=O)NC3=O)c(=O)c12 ; *c1cccc2nc(C)n([C@H]3CCC(=O)NC3=O)c(=O)c12 ; *c1cccc2nc(C)n([C@@H]3CCC(=O)NC3=O)c(=O)c12]

133. The compound of any one of claims 1-130, wherein LBM is a Von Hippel-Lindau (VHL) E3 ubiquitin ligase binding moiety.

134. The compound of any one of claims 1-130 or 133, wherein LBM is

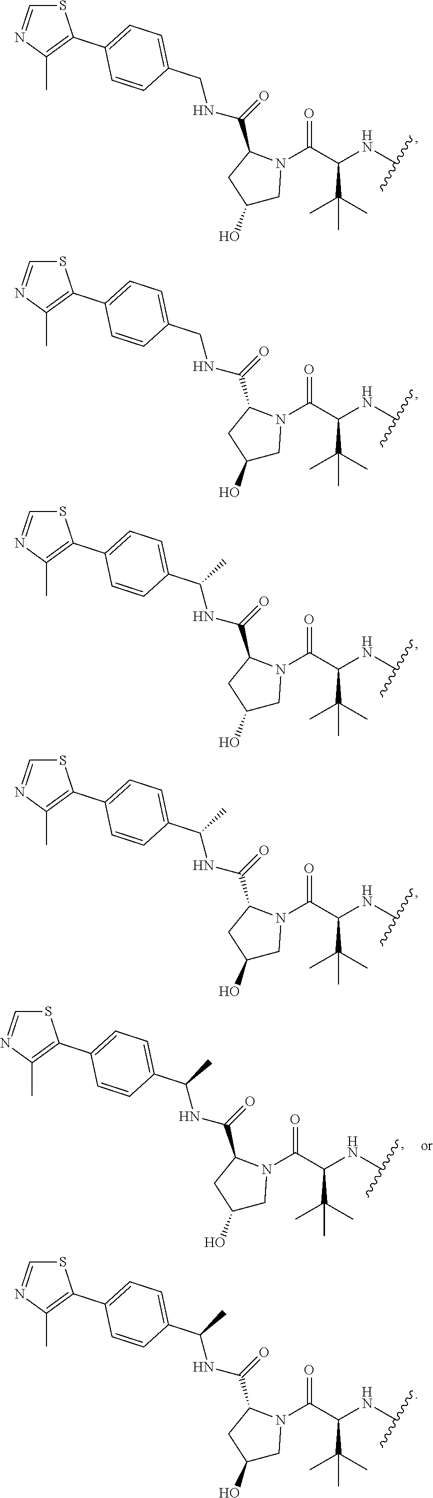 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@@H]1C(=O)NCc1ccc(-c2scnc2C)cc1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@@H](O)C[C@@H]1C(=O)N[C@@H](C)c1ccc(-c2scnc2C)cc1)C(C)(C)C ; *N[C@H](C(=O)N1C[C@H](O)C[C@H]1C(=O)N[C@H](C)c1ccc(-c2scnc2C)cc1)C(C)(C)C ; *N[C@H](C(=O)N1C[C@@H](O)C[C@@H]1C(=O)N[C@H](C)c1ccc(-c2scnc2C)cc1)C(C)(C)C]

135. The compound of any one of claims 1-129, wherein BM is PIK2.

136. The compound of any one of claims 1-129 or 135, wherein PIK2 is a PI3K binding moiety of formula I-a0: 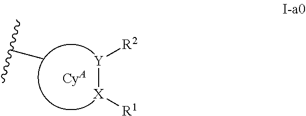
wherein:
X is C, CH, C(R^{X}), or N;
Y is C, CH, C(R^{Y}), or N;
R¹ is -L¹-R^{1A};
R² is -L²-R^{2A};
R^{X} is -L^{X}-R^{XA};
R^{Y} is -L^{Y}-R^{YA}; or
each instance of R^{CyA} is independently -L^{CyA}-R^{CyAA};
Cy^{A} is a 5-6 membered saturated, partially unsaturated, or aromatic monocyclic ring having 0-3 heteroatoms independently selected from nitrogen, oxygen, and sulfur; or a 8-10 membered saturated, partially unsaturated, or aromatic bicyclic ring having 0-4 heteroatoms independently selected from nitrogen, oxygen, and sulfur; wherein each ring is substituted with n instances of R^{CyA};
each of L¹, L², L^{X}, L^{Y}, and L^{CyA} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloallylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
R^{XA} is R^{A} or R^{B} substituted by r³ instances of R^{XC};
R^{YA} is R^{A} or R^{B} substituted by r⁴ instances of R^{YC};
R^{L} is R^{A} or R^{B} substituted by r⁵ instances of R^{LC};
each instance of R^{CyA} is independently R^{A} or R^{B} substituted by r⁶ instances of R^{CyAC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{XC}, R^{YC}, R^{LC}, and R^{CyAC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, r¹, r², r³, r⁴, r⁵, and r⁶ is independently 0, 1, 2, 3, 4, or 5.

137. The compound of any one of claims 1-129 or 135, wherein PIK2 is a PI3K binding moiety of formula I-b0: 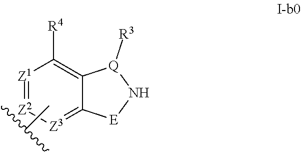
wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
Q is CH, C(R^{Q}), or N;
Z¹ is CH, C(R^{Z1}), or N;
Z² is CH, C(R^{Z2}), or N;
Z³ is CH, C(R^{Z3}), or N;
R³ is -L³-R^{3A};
R⁴ is -L⁴-R^{4A};
each instance of R^{E} is independently H or -L^{E}-R^{EA};
R^{Q} is -L^{Q}-R^{QA};
R^{Z1} is -L^{Z1}-R^{Z1A};
R^{Z2} is -L^{Z2}-R^{Z2A};
R^{Z3} is -L^{Z3}-R^{Z3A}; or
two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n¹ instances of R^{EEC};
R^{Q} and R³ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p¹ instances of R^{Q3C};
R^{Z2} and R^{Z3} are taken together with their intervening atoms to form a 4-7 membered partially unsaturated or aromatic ring having 0-4 heteroatoms independently selected from nitrogen, oxygen, and sulfur; wherein said ring is substituted with q¹ instances of R^{Z2Z3C};
each of L³, L⁴, L^{E}, L^{Q}, L^{Z1}, L^{Z2}, and L^{Z3} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L1})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{3A} is R^{C} or R^{D} substituted by s¹ instances of R^{3C};
R^{4A} is R^{C} or R^{D} substituted by s² instances of R^{4C};
R^{EA} is R^{C} or R^{D} substituted by s³ instances of R^{EC};
R^{QA} is R^{C} or R^{D} substituted by s⁴ instances of R^{QC};
R^{Z1A} is R^{C} or R^{D} substituted by s⁵ instances of R^{Z1C};
R^{Z2A} is R^{C} or R^{D} substituted by s⁶ instances of R^{Z2C};
R^{Z3A} is R^{C} or R^{D} substituted by s⁷ instances of R^{Z3C};
R^{L1} is R^{C} or R^{D} substituted by s⁸ instances of R^{L1C};
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{D} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{3C}, R^{4C}, R^{EC}, R^{QC}, R^{Z1C}, R^{Z2C}, R^{Z3C}, R^{L1C}, R^{EEC}, R^{Q3C} and R^{Z2Z3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n¹, p¹, q¹, s¹, s², s³, s⁴, s⁵, s⁶, s⁷, and s⁸ is independently 0, 1, 2, 3, 4, or 5.

138. The compound of any one of claims 1-129 or 135, wherein PIK2 is a PI3K binding moiety of formula I-c0: 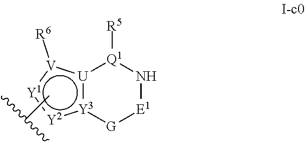
wherein:
E¹ is —C(O)—, —C(R^{E1})₂—, —C(R^{E1})₂C(R^{E1})₂—, —C(S)—, —S(O)₂—, —OC(O)—, —N(R^{E1})C(O)—, —C(O)N(R^{E1})—, or —C(R^{E1})₂C(O)—;
G is CH₂, CH(R^{G}), C(R^{G})₂, or a covalent bond;
Q¹ is CH, C(R^{Q1}), or N;
Y¹ is CH, C(R^{Y1}), or N;
Y² is CH, C(R^{Y2}), N, or N(R^{Y2});
Y³ is C or N;
U is C or N;
V is C or N; provided that at least one of Y¹, Y², Y³, U, and V is N;
R⁵ is -L⁵-R^{5A};
R⁶ is -L⁶-R^{6A};
each instance of R^{E1} is independently H or -L^{E1}-R^{E1A};
each instance of R^{G} is independently -L^{G}-R^{GA};
R^{Q1} is -L^{Q1}-R^{Q1A};
R^{Y1} is -L^{Y1}-R^{Y1A};
R^{Y2} is -L^{Y2-}R^{Y2A}; or
two instances of R^{E1} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n² instances of R^{E1EC};
R^{Q1} and R⁵ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p² instances of R^{QSC};
each of L⁵, L⁶, L^{E1}, L^{G}, L^{Q1}, L^{Y1}, and L^{Y2} is independently a covalent bond, or a C₁₋₄ bivalent saturated or unsaturated, straight or branched hydrocarbon chain wherein one or two methylene units of the chain are optionally and independently replaced by —CH(R^{L2})—, —C(R^{L2})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{5A} is R^{F} or R^{H} substituted by v¹ instances of R^{5C};
R^{6A} is R^{F} or R^{H} substituted by v² instances of R^{6C};
each instance of R^{E1A} is independently R^{F} or R^{H} substituted by v³ instances of R^{EIC};
each instance of R^{GA} is independently R^{F} or R^{H} substituted by v⁴ instances of R^{GC};
R^{Q1A} is R^{F} or R^{H} substituted by v⁵ instances of R^{Q1C};
R^{Y1A} is R^{F} or R^{H} substituted by v⁶ instances of R^{Y1C};
R^{Y2A} is R^{F} or R^{H} substituted by v⁷ instances of R^{Y2C};
R^{L2} is R^{F} or R^{H} substituted by v⁸ instances of R^{L2C};
each instance of R^{F} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{H} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{5C}, R^{6C}, R^{E1C}, R^{GC}, R^{Q1C}, R^{Y1C}, R^{Y2C}, R^{L2C}, R^{E1EC}, and R^{Q5C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n², p², v¹, v², v³, v⁴, v⁵, v⁶, v⁷, and v⁸ is independently 0, 1, 2, 3, 4, or 5.

139. The compound of any one of claims 1-129 or 135, wherein PIK2 is a PI3K binding moiety of formula I-d0 or I-d00: 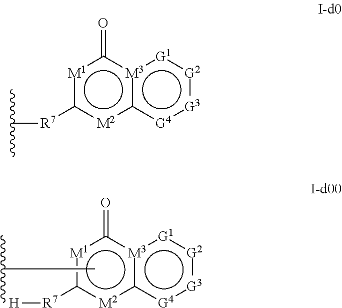
wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-BM or H;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3})—, —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z⁷ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

140. The compound of any one of claims 2-32, wherein the compound is a compound of formula I-ee1: 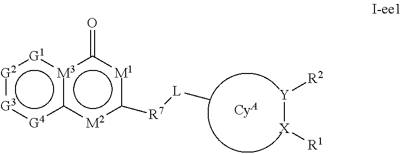
or a pharmaceutically acceptable salt thereof, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3})—, —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z¹ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

141. The compound of any one of claims 33-62, wherein the compound is a compound of formula I-ff1: 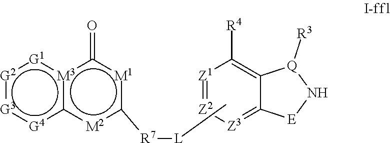
or a pharmaceutically acceptable salt thereof, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3})—, —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z¹ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

142. The compound of any one of claims 63-94, wherein the compound is a compound of formula I-gg1: 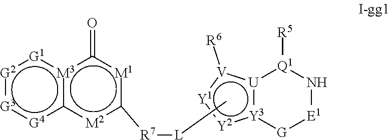
or a pharmaceutically acceptable salt thereof, wherein:
M¹ is CH, C(R^{M1}), NH, or N(R^{M1});
M² is O, CH, C(R^{M2}), N, NH, or N(R^{M2});
M³ is C or N;
G¹ is CH, N, or C—R^{G1};
G² is CH, N, or C—R^{G2};
one of G³ or G⁴ is C—R⁸ and the other is CH, N, or C—R^{G3};
R⁷ is -L⁷-R^{7A} substituted with -L-;
R⁸ is -L⁸-R^{8A};
R^{G1} is -L^{G1}-R^{G1A};
R^{G2} is -L^{G2}-R^{G2A};
R^{G3} is -L^{G3}-R^{G3A};
R^{M1} is -L^{M1}-R^{M1A};
R^{M2} is -L^{M2}-R^{M2A};
each of L⁷, L⁸, L^{G1}, L^{G2}, L^{G3}, L^{M1}, and L^{M2} is independently a covalent bond, or a C₁₋₄ bivalent saturated or unsaturated, straight or branched hydrocarbon chain wherein one or two methylene units of the chain are optionally and independently replaced by —CH(R^{L3}), —C(R^{L3})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{7A} is R^{J} or R^{K} substituted by z⁷ instances of R^{7C};
R^{8A} is R^{J}, R^{K} substituted by z² instances of R^{8C}, or Cy^{B}-R^{CyB} substituted by z² instances of R^{8C};
R^{G1A} is R^{J} or R^{K} substituted by z³ instances of R^{G1C};
R^{G2A} is R^{J} or R^{K} substituted by z⁴ instances of R^{G2C};
R^{G3A} is R^{J} or R^{K} substituted by z⁵ instances of R^{G3C};
R^{M1A} is R^{J} or R^{K} substituted by z⁶ instances of R^{M1C};
R^{M2A} is R^{J} or R^{K} substituted by z⁷ instances of R^{M2C};
R^{L3} is R^{J} or R^{K} substituted by z⁸ instances of R^{L3C};
Cy^{B} is a phenyl; naphthyl; cubanyl; adamantyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur;
R^{CyB} is R^{J} or R^{K}; or R^{CyB} and R^{8C} are taken together with their intervening atoms to form a 5-6 membered heteroaryl ring having 1-4 heteroatoms independently selected from nitrogen, oxygen, and sulfur; or a 3-7 membered partially unsaturated heterocyclic ring having 1-2 heteroatoms independently selected from nitrogen, oxygen, and sulfur;
each instance of R^{J} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{K} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{7C}, R^{8C}, R^{G1C}, R^{G2C}, R^{G3C}, R^{M1C}, R^{M2C}, and R^{L3C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of z¹, z², z³, z⁴, z⁵, z⁶, z⁷, or z⁸ is independently 0, 1, 2, 3, or 4.

143. The compound of any one of claims 1-129, wherein BM is T.

144. The compound of any one of claims 1-129 or 143, wherein T is hydrogen.

145. The compound of any one of claims 1-144, wherein L is a covalent bond or a bivalent, saturated or unsaturated, straight or branched C₁₋₅₀ hydrocarbon chain, wherein 0-10 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, 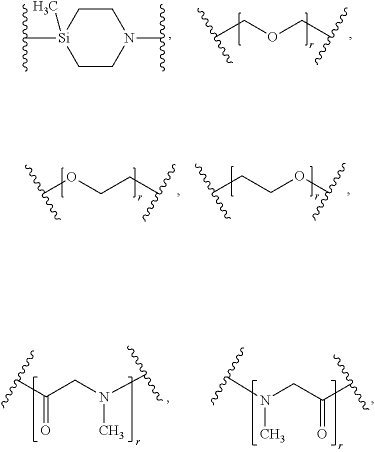
wherein: each -Cy- is independently an optionally substituted bivalent ring selected from phenylene, an 8-10 membered bicyclic arylene, a 3-7 membered saturated or partially unsaturated carbocyclylene, a 3-12 membered saturated or partially unsaturated spiro carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, a 3-12 membered saturated or partially unsaturated spiro heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, and wherein r is 0, 1, 2, 3, 4, 5, 6, 7, 8, 9, or 10.

146. The compound of any one of claims 1-144, wherein L is a bivalent, saturated or unsaturated, straight or branched C₁₋₂₀ hydrocarbon chain, wherein 0-7 methylene units of L are independently replaced by —C(H)F—, —CF₂—, —O—, —N(R)—, —S—, —C(O)—, —S(O)—, —S(O)₂—, —N(R)S(O)₂—, —S(O)₂N(R)—, and 0 or 1 methylene unit of L is replaced by phenylene, a 3-7 membered saturated or partially unsaturated carbocyclylene, or a 3-7 membered saturated or partially unsaturated heterocyclylene having 1-2 heteroatoms independently selected from nitrogen, oxygen, and sulfur.

147. The compound of any one of claims 1-144, wherein L is a bivalent, saturated or unsaturated, straight or branched C₁₋₂₀ hydrocarbon chain, wherein 0-7 methylene units of L are independently replaced by —O—, —N(R)—, or —C(O)—.

148. A compound selected from those set forth in Table 1, or a pharmaceutically acceptable salt thereof.

149. A pharmaceutical composition, comprising a compound of any one of claims 1-148, and a pharmaceutically acceptable carrier.

150. A method of inhibiting PI3Kα signaling activity in a subject, comprising administering a therapeutically effective amount of a compound of any of claims 1-148, or the pharmaceutical composition of claim 149, to a subject in need thereof.

151. A method of treating a PI3Kα-mediated disorder in a subject, comprising administering a therapeutically effective amount of a compound of any of claims 1-148, or the pharmaceutical composition of claim 149, to a subject in need thereof.

152. A method of treating a cellular proliferative disease in a subject, comprising administering a therapeutically effective amount of a compound of any of claims 1-148, or the pharmaceutical composition of claim 149, to a subject in need thereof.

153. The method of claim 152, wherein the cellular proliferative disease is cancer.

154. The method of claim 153, wherein the cancer is breast cancer.

155. The method of claim 154, wherein the cancer is ovarian cancer.

156. The method of claim 155, wherein the ovarian cancer is clear cell ovarian cancer.

157. The method of any one of claims 150-156, wherein the subject has PI3Kα containing at least one of the following mutations: H1047R, H1047L, E542K, and E545K.
